(19) United States
(12) Patent Application Publication
(10) Pub. No.: US 2025/0064945 A1
(43) Pub. Date: Feb. 27, 2025

(54) BRM TARGETING COMPOUNDS AND ASSOCIATED METHODS OF USE

(71) Applicants: Arvinas Operations, Inc., New Haven, CT (US); Genentech, Inc., South San Francisco, CA (US)

(72) Inventors: Michael Berlin, Flemington, NJ (US); Huifen Chen, South San Francisco, CA (US); Peter Scott Dragovich, South San Francisco, CA (US); Leanna Renee Staben, South San Francisco, CA (US); Jing Wang, Milford, CT (US)

(21) Appl. No.: 18/709,947

(22) PCT Filed: Nov. 23, 2022
(86) PCT No.: PCT/US2022/050888

§ 371 Date: May 14, 2024

Related U.S. Application Data
(60) Provisional application No. 63/282,891, filed on Nov. 24, 2021.

(51) Int. Cl. A61K 47/55; A61K 31/519; A61K 47/54; C07D 487/04; C07D 519/00

(52) U.S. Cl. CPC A61K 47/55; A61K 31/519; A61K 47/545; C07D 487/04; C07D 519/00

(57) ABSTRACT

The present disclosure relates to bifunctional compounds, which find utility as modulators of SMARCA2 or BRM (target protein). In particular, the present disclosure is directed to bifunctional compounds, which contain on one end a ligand that binds to the Von Hippel-Lindau E3 ubiquitin ligase, and on the other end a moiety which binds the target protein, such that the target protein is placed in proximity to the ubiquitin ligase to effect degradation (and inhibition) of target protein. The present disclosure exhibits a broad range of pharmacological activities associated with degradation/inhibition of target protein. Diseases or disorders that result from aggregation or accumulation of the target protein are treated or prevented with compounds and compositions of the present disclosure.

### RELATED APPLICATIONS

This application claims priority to U.S. Provisional Application No. 63/282,891, filed on Nov. 24, 2021. The entire contents of the foregoing application are expressly incorporated herein by reference.

### FIELD OF THE INVENTION

The description provides novel compound that binds and/or inhibits Switch/Sucrose Non Fermentable (SWI/SNF)-Related, Matrix-Associated, Actin-Dependent Regulator of Chromatin, Subfamily A, Member 2 (SMARCA2) (i.e., BRAHMA or BRM), as well as bifunctional compounds comprising a target protein binding moiety and a E3 ubiquitin ligase binding moiety, and associated methods of use. The bifunctional compounds are useful as modulators of targeted ubiquitination, especially with respect to BRM, which are degraded and/or otherwise inhibited by bifunctional compounds according to the present disclosure.

### BACKGROUND

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore, are more attractive therapeutic targets than general proteasome inhibitors due to their specificity for certain protein substrates. The development of ligands of E3 ligases has proven challenging, in part due to the fact that they must disrupt protein-protein interactions. However, recent developments have provided specific ligands which bind to these ligases. For example, since the discovery of nutlins, the first small molecule E3 ligase inhibitors, additional compounds have been reported that target E3 ligases but the field remains underdeveloped. For example, since the discovery of Nutlins, the first small molecule E3 ligase mouse double minute 2 homolog (MDM2) inhibitors, additional compounds have been reported that target MDM2 (i.e., human double minute 2 or HDM2) E3 ligases (J. Di, et al. Current Cancer Drug Targets (2011), 11(8), 987-994).

One E3 ligase with exciting therapeutic potential is the von Hippel-Lindau (VHL) tumor suppressor, the substrate recognition subunit of the E3 ligase complex VCB, which also consists of elongins B and C, Cul2 and Rbx1. The primary substrate of VHL is Hypoxia Inducible Factor 1α (HIF-1α), a transcription factor that upregulates genes such as the pro-angiogenic growth factor VEGF and the red blood cell inducing cytokine erythropoietin in response to low oxygen levels. The first small molecule ligands of Von Hippel Lindau (VHL) to the substrate recognition subunit of the E3 ligase were generated, and crystal structures were obtained confirming that the compound mimics the binding mode of the transcription factor HIF-1α, the major substrate of VHL.

Bifunctional compounds such as those that are described in U.S. Patent Application Publications 2015-0291562 and 2014-0356322 (incorporated herein by reference), function to recruit endogenous proteins to an E3 ubiquiuin ligase for degradation. In particular, the publications describe bifunctional or proteolysis targeting chimeric (PROTAC®) compounds, which find utility as modulators of targeted ubiquitination of a variety of polypeptides and other proteins, which are then degraded and/or otherwise inhibited by the bifunctional compounds.

The Switch/Sucrose Non Fermentable (SWI/SNF) is a multi-subunit complex that modulates chromatic structure through the activity of two mutually exclusive helicase/ATPase catalytic subunits SWI/SNF-Related, Matrix-Associated, Actin-Dependent Regulator of Chromatin, Subfamily A, Member 2 (SMARCA2, BRAHMA or BRM) and SWI/SNF-Related, Matrix-Associated, Actin-Dependent Regulator of Chromatin, Subfamily A, Member 4 (SMARCA4 or BRQ1). The core and the regulatory subunits couple ATP hydrolysis to the perturbation of histone-DNA contacts, thereby providing access points to transcription factors and cognate DNA elements that facilitate gene activation and repression.

Mutations in the genes encoding the twenty canonical SWI/SNF subunits are observed in nearly 20% of all cancers with the highest frequency of mutations observed in rhabdoid tumors, female cancers (including ovarian, uterine, cervical, and endometrial), lung adenocarcinoma, gastric adenocarcinoma, melanoma, esophageal, and renal clear cell carcinoma. Despite having a high degree of homology, and their presumed overlapping functions, SMARCA2 and SMARCA4 have been reported as having distinct roles in cancer. For example, SMARCA4 is frequently mutated in primary tumors, while SMARCA2 inactivation is infrequent in tumor development. In fact, numerous types of cancer have been shown to be SMARCA4-related (e.g., cancers having a SMARCA4-mutation or a SMARCA4-deficiency, such as lack of expression), including, e.g., lung cancer (such as non-small cell lung cancer).

SMARCA2 is one of the top essential genes in SMARCA4-related or-mutant cancer cell lines because SMARCA4-deficient patient populations or cells depend exclusively on SMARCA2 activity—i.e., there is a greater incorporation of SMARCA2 into the complex to compensate for the SMARCA4 deficiency. Thus, SMARCA2 may be targeted in SMARCA4-related/deficient cancers. The co-occurrence of the deficiency of the expression of two (or more) genes that leads to cell death is known as, synthetic lethality. Accordingly, synthetic lethality can be leveraged in the treatment of certain SMARCA2/SMARCA4-related cancers.

There is an ongoing need for effective treatment for diseases that are treatable by inhibiting or degrading SMARCA2 (i.e., BRAHMA or BRM). However, non-specific effects, and the inability to target and modulate SMARCA2 remain obstacles to developing effective treatments. Accordingly, there remains a need in the art for novel and effective small-molecule therapeutic agents that target SMARCA2 and that leverage or potentiate VHL's substrate specificity.

### SUMMARY

Disclosed herein are novel compounds that binds and/or inhibits Switch/Sucrose Non Fermentable (SWI/SNF)-Related, Matrix-Associated, Actin-Dependent Regulator of Chromatin, Subfamily A, Member 2 (SMARCA2) (i.e., BRAHMA or BRM), as well as bifunctional compounds comprising a target protein binding moiety and a E3 ubiquitin ligase binding moiety. The bifunctional compounds are useful as modulators of targeted ubiquitination, especially with respect to BRM, which are degraded and/or otherwise inhibited by bifunctional compounds according to the present disclosure.

The present disclosure describes bifunctional compounds which function to recruit endogenous proteins to an E3 ubiquitin ligase for degradation, and methods of using the same. In particular, the present disclosure provides bifunctional or proteolysis targeting chimeric (PROTAC®) compounds, which find utility as modulators of targeted ubiquitination of a variety of polypeptides and other proteins, which are then degraded and/or otherwise inhibited by the bifunctional compounds as described herein. An advantage of the compounds provided herein is that a broad range of pharmacological activities is possible, consistent with the degradation/inhibition of targeted polypeptides from virtually any protein class or family. In addition, the description provides methods of using an effective amount of the compounds as described herein for the treatment or amelioration of a disease condition, such as cancer, e.g., SMARCA4-related/deficient cancer, such as lung cancer or non-small cell lung cancer.

As such, in one aspect the disclosure provides bifunctional compounds, which comprise an E3 ubiquitin ligase binding moiety (i.e., a ligand for an E3 ubiquitin ligase or “ULM” group), and a moiety that binds a target protein (i.e., a protein/polypeptide targeting ligand or “PTM” group) such that the target protein/polypeptide is placed in proximity to the ubiquitin ligase to effect degradation (and inhibition) of that protein. In a preferred embodiment, the ULM (ubiquitination ligase modulator) can be Von Hippel-Lindau E3 ubiquitin ligase (VHL) binding moiety (VLM). For example, the structure of the bifunctional compound can be depicted as:

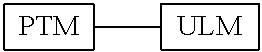

The respective positions of the PTM and ULM moieties as well as their number as illustrated herein is provided by way of example only and is not intended to limit the compounds in any way. As would be understood by the skilled artisan, the bifunctional compounds as described herein can be synthesized such that the number and position of the respective functional moieties can be varied as desired.

In certain embodiments, the bifunctional compound further comprises a chemical linker (“L”). In this example, the structure of the bifunctional compound can be depicted as:

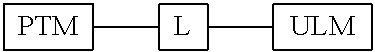

where PTM is a protein/polypeptide targeting moiety, L is a linker, e.g., a bond or a chemical group coupling PTM to ULM, and ULM is a Von Hippel-Lindau E3 ubiquitin ligase (VHL) binding moiety (VLM).

For example, the structure of the bifunctional compound can be depicted as:

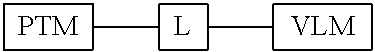

wherein: PTM is a protein/polypeptide targeting moiety; “L” is a linker (e.g. a bond or a chemical linker group) coupling the PTM and a VLM, wherein VLM is Von Hippel-Lindau E3 ubiquitin ligase binding moiety that binds to VHL E3 ligase.

In certain embodiments, the compounds as described herein comprise multiple independently selected ULMs, multiple PTMs, multiple chemical linkers or a combination thereof.

In additional embodiments, VLM can be hydroxyproline or a derivative thereof. Furthermore, other contemplated VLMs are included in U.S. Patent Application Publication No. 2014/03022523, which as discussed above, is incorporated herein in its entirety.

In certain embodiments, “L” is a bond. In additional embodiments, the linker “L” is a connector with a linear non-hydrogen atom number in the range of 1 to 20. The connector “L” can contain, but not limited to the functional groups such as ether, amide, alkane, alkene, alkyne, ketone, hydroxyl, carboxylic acid, thioether, sulfoxide, and sulfone. The linker can contain aromatic, heteroaromatic, cyclic, bicyclic and tricyclic moieties. Substitution with halogen, such as Cl, F, Br and I can be included in the linker. In the case of fluorine substitution, single or multiple fluorines can be included.

In certain embodiments, VLM is a derivative of trans-3-hydroxyproline, where both nitrogen and carboxylic acid in trans-3-hydroxyproline are functionalized as amides.

In an additional aspect, the description provides therapeutic compositions comprising an effective amount of a compound as described herein or salt form thereof, and a pharmaceutically acceptable carrier. The therapeutic compositions modulate protein degradation and/or inhibition in a patient or subject, for example, an animal such as a human, and can be used for treating or ameliorating disease states or conditions which are modulated through the degraded/inhibited protein. In certain embodiments, the therapeutic compositions as described herein may be used to effectuate the degradation of proteins of interest for the treatment or amelioration of a disease, e.g., cancer (including at least one of SWI/SNF associated cancer, a cancer with a SMARCA4 mutation, a cancer with a SMARCA4-deficiency, or a combination thereof), such as lung cancer (e.g., non-small cell lung cancer). In yet another aspect, the present disclosure provides a method of ubiquitinating/degrading a target protein in a cell. In certain embodiments, the method comprises administering a bifunctional compound as described herein comprising a VLM, preferably linked through a linker moiety, as otherwise described herein, wherein the VLM is coupled to the PTM through a linker to target a protein for degradation. Degradation of the target protein will occur when the target protein is placed in proximity to the E3 ubiquitin ligase, thus resulting in degradation/inhibition of the effects of the target protein and the control of protein levels. The control of protein levels afforded by the present disclosure provides treatment of a disease state or condition, which is modulated through the target protein by lowering the level of that protein in the cells of a patient.

In still another aspect, the description provides methods for treating or ameliorating a disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable carrier, wherein the composition is effective for treating or ameliorating the disease or disorder or symptom thereof in the subject.

In another aspect, the description provides methods for identifying the effects of the degradation of proteins of interest in a biological system using compounds according to the present disclosure.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional aspects and embodiments are expressly included within the scope of the present disclosure. The publications and other materials used herein to illuminate the background of the disclosure, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating an embodiment of the disclosure and are not to be construed as limiting the disclosure. Further objects, features and advantages of the disclosure will become apparent from the following detailed description taken in conjunction with the accompanying figures showing illustrative embodiments of the disclosure, in which:

FIGS. 1A and 1B. Illustration of general principle for bifunctional degradative compound function. (A) Exemplary bifunctional degradative compounds comprise a protein targeting moiety (PTM; darkly shaded rectangle), a ubiquitin ligase binding moiety (ULM; lightly shaded triangle), and optionally a linker moiety (L; black line) coupling or tethering the PTM to the ULM. (B) Illustrates the functional use of the bifunctional degradative compounds as described herein. Briefly, the ULM recognizes and binds to a specific E3 ubiquitin ligase, and the PTM binds and recruits a target protein bringing it into close proximity to the E3 ubiquitin ligase. Typically, the E3 ubiquitin ligase is complexed with an E2 ubiquitin-conjugating protein, and either alone or via the E2 protein catalyzes attachment of ubiquitin (dark circles) to a lysine on the target protein via an isopeptide bond. The poly-ubiquitinated protein (far right) is then targeted for degradation by the proteosomal machinery of the cell.

### DETAILED DESCRIPTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

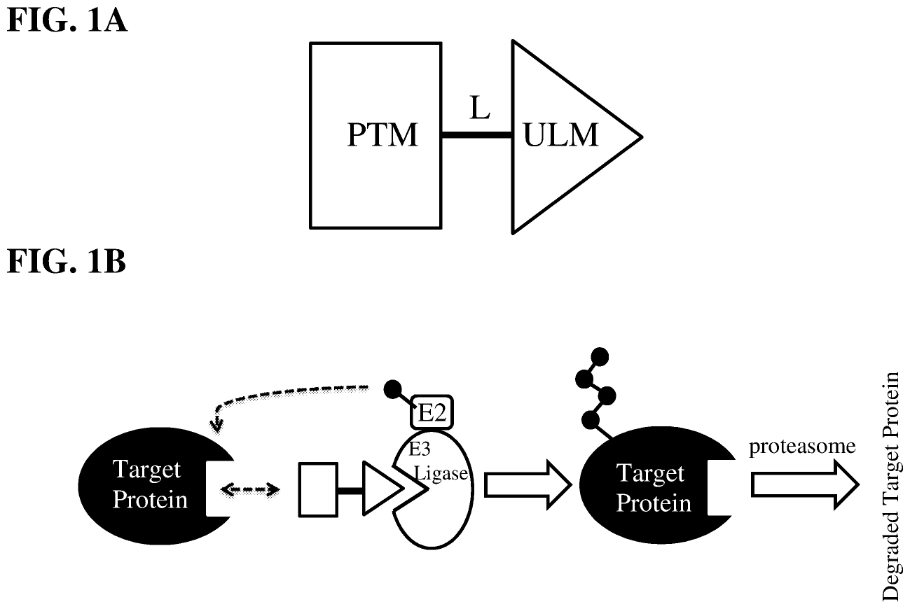

Presently described are compositions and methods that relate to the surprising and unexpected discovery that an E3 ubiquitin ligase protein (e.g., Von Hippel-Lindau E3 ubiquitin ligase (VHL)) ubiquitinates a target protein once it and the target protein are placed in proximity by a bifunctional or chimeric construct that binds the E3 ubiquitin ligase protein and the target protein. Accordingly the present disclosure provides such compounds and compositions comprising an E3 ubiquitin ligase binding moiety (“ULM”) coupled to a protein target binding moiety (“PTM”), which result in the ubiquitination of a chosen target protein and leads to degradation of the target protein by the proteasome (see FIG. 1). The present disclosure also provides a library of compositions and uses thereof.

In certain aspects, the present disclosure provides compounds which comprise a ligand, e.g., a small molecule ligand (i.e., having a molecular weight of below 2,000 Daltons, 1,000 Daltons, 500 Daltons, or 200 Daltons) that is capable of binding to a ubiquitin ligase, such as VHL. The compounds also comprise a moiety that is capable of binding to target protein, in such a way that the target protein is placed in proximity to the ubiquitin ligase to effect degradation (and/or inhibition) of that protein. As disclosed herein, the term “small molecule” means, in addition to the above, a molecule that is non-peptidyl, that is, it is not generally considered a peptide, e.g., comprises fewer than 4 amino acids, 3 amino acids, or 2 amino acids). In accordance with the present description, the PTM, ULM, or bifunctional compounds disclosed herein can be small molecules.

### Definitions

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The terms “co-administration,” “co-administering,” and “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are coadministered in combination with at least one additional bioactive agent, especially including an anticancer agent. In particularly preferred aspects, the co-administration of compounds results in synergistic activity and/or therapy, including anticancer activity.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives, including prodrug and/or deuterated forms thereof where applicable, in context. Deuterated small molecules contemplated are those in which one or more of the hydrogen atoms contained in the drug molecule have been replaced by deuterium.

Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules that are described herein are stable. When the bond is shown, both a double bond and single bond are represented or understood within the context of the compound shown and well-known rules for valence interactions.

The term “ubiquitin ligase” refers to a family of proteins that facilitate the transfer of ubiquitin to a specific substrate protein, targeting the substrate protein for degradation. For example, an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome. As used herein, the term “alkyl”, by itself or as part of another substituent, means, unless otherwise stated, a straight or branched chain hydrocarbon radical, having the number of carbon atoms designated (i.e., C1-8 means one to eight carbons). Absent a specific number of carbon atoms, an alkyl group provided herein is assumed to have one to twelve carbons, one to eight carbons, one to six carbons, or one to four carbons. Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. Alkyl groups may be optionally substituted as provided herein. In some embodiments, the alkyl group is C1-6 alkyl; in some embodiments, the alkyl group is C1-4 alkyl.

The term “optionally substituted,” as used in combination with a substituent defined herein, means that the substituent may, but is not required to be, substituted with one or more suitable functional groups or other substituents as provided herein. For example, a substituent may be optionally substituted with one or more of: halo, cyano, C₁₋₆ alkyl, C₃₋₆ cycloalkyl, C₂₋₆ alkenyl, C₂₋₆ alkynyl, halo(C₁₋₆)alkyl, C₁₋₆ alkoxy, halo(C₁₋₆alkoxy), C₁₋₆ alkylthio, C₁₋₆ alkylamino, NH₂, NH(C₁₋₆ alkyl), N(C₁₋₆ alkyl)₂, NH(C₁₋₆ alkoxy), N(C₁₋₆ alkoxy)₂, —C(O)NHC₁₋₆ alkyl, —C(O)N(C₁₋₆ alkyl)₂, —C(O)NH₂, —C(O)C₁₋₆ alkyl, —C(O)₂C₁₋₆ alkyl, —NHCO(C₁₋₆ alkyl), —N(C₁₋₆ alkyl)CO(C₁₋₆ alkyl), —S(O)C₁₋₆ alkyl, —S(O)₂C₁₋₆ alkyl, oxo, phenyl, benzyl, pyridinyl, pyrrazolyl, thiazolyl, isothiazolyl, or other 5 to 6 membered heteroaryl groups. In some embodiments, each of the above optional substituents are themselves optionally substituted by one or two groups.

The term “cycloalkyl” as used herein refers to a C3-12 cyclic alkyl group, and includes bridged and spirocycles (e.g., adamantine). Examples of cycloalkyl groups include cyclopropyl, cyclobutyl, cyclopentyl, cycloheptyl, cyclohexyl, cycloheptyl, cyclooctyl, bicyclo[2.2.1]heptanyl, bicyclo[3.1.1]heptanyl, bicyclo[4.1.0]heptanyl, spiro[3.3]heptanyl, and spiro[3.4]octanyl. In some embodiments, the cycloalkyl group is a C3-6 cycloalkyl.

The term “akenyl,” as used herein, refers to C2-12 alkyl group wherein at least two of the carbon atoms are sp2 hybridized and form a carbon-carbon double bond between them. An alkenyl group provided herein may contain more than one carbon-carbon double bond, but one is preferred. The alkyl portion of an alkenyl group provided herein may be substituted as provided above. In some embodiments, the alkenyl group is a C2-6 alkenyl.

The term “akynyl” as used herein refers to C2-12 alkyl group wherein at least two of the carbon atoms are sp hybridized and form a carbon-carbon triple bond between them. An alkynyl group provided herein may contain more than one carbon-carbon triple bond, but one is preferred. The alkyl portion of an alkynyl group provided herein may be substituted as provided above. In some embodiments, the alkynyl group is a C2-6 alkynyl.

The terms “alkoxy,” “alkylamino,” and “alkylthio” are used in their conventional sense, and refer to those alkyl groups attached to the remainder of the molecule via an oxygen atom (“oxy”), an amino group (“amino”) or thio group. The term “alkylamino” includes mono-di-alkylamino groups, the alkyl portions can be the same or different.

The terms “halo” and “halogen” by alone or as part of another substituent, mean, unless otherwise stated, fluorine, chlorine, bromine, or iodine, but preferably fluorine or chlorine.

The term “halo(C1-x alkyl)” refers to an alkyl that has 1-x carbon atoms and that is substituted with one or more (e.g., 1, 2, 3, 4, 5, or 6) halo groups. For example the term includes an alkyl group having 1-6 carbon atoms that is substituted with one or more halo groups. Non-limiting examples of the term halo(C1-6alkyl) include fluoromethyl, difluoromethyl, trifluoromethyl, chloromethyl, and 2,2,2-trifluoroethyl.

The term “halo(C1-x alkoxy)” refers to an alkoxy group that has 1-x carbon atoms and that is substituted with one or more (e.g., 1, 2, 3, 4, 5, or 6) halo groups. For example the term includes an alkoxy group having 1-6 carbon atoms that is substituted with one or more halo groups. Non-limiting examples of the term halo(C1-6alkyl) include fluoromethoxy, difluoromethoxy, trifluoromethoxy, chloromethoxy, and 2,2,2-trifluoroethoxy.

The term “heteroalkyl” refers to a straight- or branched-chain alkyl group, e.g., having from 2 to 14 carbons, such as 2 to 10 carbons, in the chain, one or more of which has been replaced by a heteroatom selected from S, O, P, and N. Exemplary heteroalkyls include alkyl ethers, secondary and tertiary alkyl amines, alkyl amides, alkyl sulfides, and the like. The group may be a terminal group or a bridging group. As used herein reference to the normal chain when used in the context of a bridging group refers to the direct chain of atoms linking the two terminal positions of the bridging group.

The term “aryl” as used herein refers to a single all carbon aromatic ring or a multiple condensed all carbon ring system wherein at least one of the rings is aromatic. For example, in certain embodiments, an aryl group has 6 to 12 carbon atoms. Aryl includes a phenyl radical. Aryl also includes multiple condensed ring systems (e.g., ring systems comprising 2 rings, 3 rings, or 4 rings) having about 9 to 12 carbon atoms in which at least one ring is aromatic and wherein the other rings may be aromatic or not aromatic. Such multiple condensed ring systems are optionally substituted with one or more (e.g., 1, 2, or 3) oxo groups on any carbocycle portion of the multiple condensed ring system. The rings of the multiple condensed ring system can be connected to each other via fused, spiro, and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, naphthyl, 1-, 2-, 3-, 4-tetrahydronaphthyl, and the like.

The term “heteroaryl,” as used herein, refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms also may be present in an oxidized form provided the ring is aromatic. Exemplary heteroaryl ring systems include but are not limited to pyridyl, pyrimidinyl, oxazolyl, or furyl. “Heteroaryl” also includes multiple condensed ring systems (e.g., ring systems comprising 2, 3, or 4 rings) wherein a heteroaryl group, as defined above, is condensed with one or more rings selected from heteroaryls (to form for example a naphthyridinyl such as 1,8-naphthyridinyl), heterocycles, (to form for example a 1-, 2-, 3-, 4-tetrahydronaphthyridinyl such as 1-, 2-, 3-, 4-tetrahydro-1,8-naphthyridinyl), carbocycles (to form for example 5-, 6-, 7-, 8-tetrahydroquinolyl) and aryls (to form, for example, indazolyl) to form the multiple condensed ring system. Thus, a heteroaryl (a single aromatic ring or multiple condensed ring system) has about 1-20 carbon atoms and about 1-6 heteroatoms within the heteroaryl ring. A heteroaryl (a single aromatic ring or multiple condensed ring system) can also have about 5 to 12 or about 5 to 10 members within the heteroaryl ring. Multiple condensed ring systems may be optionally substituted with one or more (e.g., 1, 2, 3 or 4) oxo groups on the carbocycle or heterocycle portions of the condensed ring. The rings of a multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system. It is also to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include, but are not limited to, pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole and 3b,4,4a,5-tetrahydro-1H-cyclopropa[3,4]cyclo-penta[1,2-c]pyrazole. In one embodiment the term “heteroaryl” refers to a single aromatic ring containing at least one heteroatom. For example, the term includes 5-membered and 6-membered monocyclic aromatic rings that include one or more heteroatoms. Non-limiting examples of heteroaryl include but are not limited to pyridyl, furyl, thiazole, pyrimidine, oxazole, and thiadiazole.

The term “heterocyclyl” or “heterocycle” as used herein refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3-, 4-, 5-, 6-, or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2 rings, 3 rings, or 4 rings) wherein a single heterocycle ring (as defined above) can be condensed with one or more groups selected from heterocycles (to form for example a 1,8-decahydronapthyridinyl), carbocycles (to form for example a decahydroquinolyl) and aryls to form the multiple condensed ring system. Thus, a heterocycle (a single saturated or single partially unsaturated ring or multiple condensed ring system) has about 2-20 carbon atoms and 1-6 heteroatoms within the heterocycle ring. Such multiple condensed ring systems may be optionally substituted with one or more (e.g., 1, 2, 3, or 4) oxo groups on the carbocycle or heterocycle portions of the multiple condensed ring. The rings of the multiple condensed ring system can be connected to each other via fused, spiro and bridged bonds when allowed by valency requirements. It is to be understood that the individual rings of the multiple condensed ring system may be connected in any order relative to one another. Accordingly, a heterocycle (a single saturated or single partially unsaturated ring or multiple condensed ring system) has about 3-20 atoms including about 1-6 heteroatoms within the heterocycle ring system. It is also to be understood that the point of attachment of a multiple condensed ring system (as defined above for a heterocycle) can be at any position of the multiple condensed ring system including a heterocycle, aryl and carbocycle portion of the ring. It is also to be understood that the point of attachment for a heterocycle or heterocycle multiple condensed ring system can be at any suitable atom of the heterocycle or heterocycle multiple condensed ring system including a carbon atom and a heteroatom (e.g., a nitrogen). In one embodiment the term heterocycle includes a C2-20 heterocycle. In one embodiment the term heterocycle includes a C2-7 heterocycle. In one embodiment the term heterocycle includes a C2-5 heterocycle. In one embodiment the term heterocycle includes a C2-4 heterocycle. Exemplary heterocycles include, but are not limited to, aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one N-methylpiperidine, imidazolidine, pyrazolidine, butyrolactam, valerolactam, imidazolidinone, hydantoin, dioxolane, phthalimide, 1,4-dioxane, thiomorpholine, thiomorpholine-S-oxide, thiomorpholine-S,S-oxide, pyran, 3-pyrroline, thiopyran, pyrone, tetrhydrothiophene, quinuclidine, tropane, 2-azaspiro[3.3]heptane, (1R,5S)-3-azabicyclo[3.2.1]octane, (1s,4s)-2-azabicyclo[2.2.2]octane, (1R,4R)-2-oxa-5-azabicyclo[2.2.2]octane and pyrrolidin-2-one. In one embodiment the term “heterocycle” refers to a monocyclic, saturated or partially unsaturated, 3-8 membered ring having at least one heteroatom. For example, the term includes a monocyclic, saturated or partially unsaturated, 4, 5, 6, or 7 membered ring having at least one heteroatom. Non-limiting examples of heterocycle include aziridine, azetidine, pyrrolidine, piperidine, piperidine, piperazine, oxirane, morpholine, and thiomorpholine. The term “9- or 10-membered heterobicycle” as used herein refers to a partially unsaturated or aromatic fused bicyclic ring system having at least one heteroatom. For example, the term 9- or 10-membered heterobicycle includes a bicyclic ring system having a benzo ring fused to a 5-membered or 6-membered saturated, partially unsaturated, or aromatic ring that contains one or more heteroatoms.

As used herein, the term “heteroatom” is meant to include oxygen (O), nitrogen (N), sulfur (S) and silicon (Si). The nitrogen and sulfur can be in an oxidized form when feasible.

As used herein, the term “chiral” refers to molecules that have the property of non-superimposability of the mirror image partner, while the term “achiral” refers to molecules that are superimposable on their mirror image partner.

As used herein, the term “stereoisomers” refers to compounds that have identical chemical constitution, but differ with regard to the arrangement of the atoms or groups in space. As used herein a crossed line “ [symbol] ” indicates a mixture of E and Z stereoisomers.

As used herein a wavy line “ [symbol] ” or a dashed line “ [symbol] ” that intersects a bond in a chemical structure indicates the point of attachment of the bond that the wavy bond intersects in the chemical structure to the remainder of a molecule.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers can separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

When a bond in a compound formula herein is drawn in a non-stereochemical manner (e.g., flat), the atom to which the bond is attached includes all stereochemical possibilities. When a bond in a compound formula herein is drawn in a defined stereochemical manner (e.g. bold, bold-wedge, dashed, or dashed-wedge), it is to be understood that the atom to which the stereochemical bond is attached is enriched in the absolute stereoisomer depicted unless otherwise noted. In some embodiments, the compound is at least 51% the absolute stereoisomer depicted. In some embodiments, the compound is at least 80% the absolute stereoisomer depicted. In some embodiments, the compound is at least 90% the absolute stereoisomer depicted. In some embodiments, the compound is at least 95% the absolute stereoisomer depicted. In some embodiments, the compound is at least 97% the absolute stereoisomer depicted. In some embodiments, the compound is at least 98% the absolute stereoisomer depicted. In some embodiments, the compound is at least 99% the absolute stereoisomer depicted.

As used herein, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

As used herein, the term “solvate” refers to an association or complex of one or more solvent molecules and a compound of the invention. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water.

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4th edition, Wiley-Interscience, New York, 2006.

As used herein, the term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. As used herein the term “prodrug” refers to those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Prodrugs of the invention include compounds wherein an amino acid residue, or a polypeptide chain of two or more (e.g., two, three or four) amino acid residues, is covalently joined through an amide or ester bond to a free amino, hydroxy or carboxylic acid group of a compound of the present invention. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, citrulline, homocysteine, homoserine, methyl-alanine, para-benzoylphenylalanine, phenylglycine, propargylglycine, sarcosine, methionine sulfone and tert-butylglycine.

Additional types of prodrugs are also encompassed. For instance, a free carboxyl group of a compound of the invention can be derivatized as an amide or alkyl ester. As another example, compounds of this invention comprising free hydroxy groups can be derivatized as prodrugs by converting the hydroxy group into a group such as, but not limited to, a phosphate ester, hemisuccinate, dimethylaminoacetate, or phosphoryloxymethyloxycarbonyl group, as outlined in Fleisher, D. et al., (1996) Improved oral drug delivery: solubility limitations overcome by the use of prodrugs Advanced Drug Delivery Reviews, 19:115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers, wherein the acyl group can be an alkyl ester optionally substituted with groups including, but not limited to, ether, amine and carboxylic acid functionalities, or where the acyl group is an amino acid ester as described above, are also encompassed. Prodrugs of this type are described in J. Med. Chem., (1996), 39:10. More specific examples include replacement of the hydrogen atom of the alcohol group with a group such as (C1-6)alkanoyloxymethyl, 1-((C1-6)alkanoyloxy)ethyl, 1-methyl-1-((C1-6)alkanoyloxy)ethyl, (C1-6)alkoxycarbonyloxymethyl, N—(C1-6)alkoxycarbonylaminomethyl, succinoyl, (C1-6)alkanoyl, alpha-amino(C1-4)alkanoyl, arylacyl and alpha-aminoacyl, or alpha-aminoacyl-alpha-aminoacyl, where each alpha-aminoacyl group is independently selected from the naturally occurring L-amino acids, P(O)(OH)2, —P(O)(O(C1-6)alkyl)2 or glycosyl (the radical resulting from the removal of a hydroxyl group of the hemiacetal form of a carbohydrate).

For additional examples of prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8:1-38 (1992); d) H. Bundgaard, et al., Journal of Pharmaceutical Sciences, 77:285 (1988); and e) N. Kakeya, et al., Chem. Pharm. Bull., 32:692 (1984), each of which is specifically incorporated herein by reference.

Additionally, the present invention provides for metabolites of compounds of the invention. As used herein, a “metabolite” refers to a product produced through metabolism in the body of a specified compound or salt thereof. Such products can result for example from the oxidation, reduction, hydrolysis, amidation, deamidation, esterification, deesterification, enzymatic cleavage, and the like, of the administered compound.

Metabolite products typically are identified by preparing a radiolabelled (e.g., 14C or 3H) isotope of a compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the invention.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

### Compounds

BRM Protein Binding and/or Inhibiting Compounds

Disclosed herein are novel BRM protein binding and/or inhibiting compounds, wherein the compound has the chemical structure:

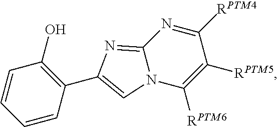

wherein:
R^{PTM4} is selected from H, —NH₂, —OH, or —NR^{PTM7}R^{PTM8}, wherein R^{PTM7} and R^{PTM8} are independently selected C₁₋₄ alkyls; and
one of R^{PTM5} and R^{PTM6} is H, and the other is selected from a H, 5- or 6-membered aryl, 5- or 6-membered heteroaryl (e.g.,

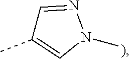

an optionally substituted 4-8-membered heterocycloalkyl (e.g. a 4-8 membered heterocycloalkyl substituted with 0, 1, or 2 substituents selected from hydroxy, halogen, alkoxy, alkyl, haloalkyl, amino, alkylamino and cyano; or

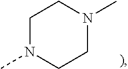

optionally substituted 8-10 membered bridged biheterocyclyl rings (e.g. a 8-10 membered bridged bicycloalkyl or bridged biheterocyclyl rings substituted with 0, 1, or 2 substituents selected from hydroxy, halogen, alkoxy, alkyl, haloalkyl, amino, alkylamino and cyano;

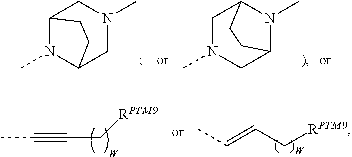

wherein W is 0, 1, or 2, and R^{PTM9} is selected from a H, alkyl (e.g., methyl), methoxy, optionally substituted 5- or 6-membered aryl, optionally substituted 5- or 6-membered heteroaryl (e.g.,

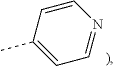

or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, R^{PTM4} is selected from H, —NH₂, or —OH, or —NR^{PTM7}R^{PTM8}, wherein R^{PTM7} and R^{PTM8} are independently selected C₁-C₄ alkyls.

In any aspect or embodiment described herein, R^{PTM4} is selected from H, —NH₂, or —OH.

In any aspect or embodiment described herein, one of R^{PTM5} and R^{PTM6} is H, and the other is selected from a H, 5- or 6-membered aryl, 5- or 6-membered heteroaryl (e.g.,

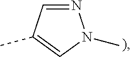

an optionally substituted 4-8-membered heterocycloalkyl (e.g. a 4-8 membered heterocycloalkyl substituted with 0, 1, or 2 substituents selected from hydroxy, halogen, alkoxy, alkyl, haloalkyl, amino, alkylamino and cyano; or

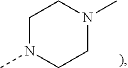

optionally substituted 8-10 membered bridged biheterocyclyl rings (e.g. a 8-10 membered bridged bicycloalkyl or bridged biheterocyclyl rings substituted with 0, 1, or 2 substituents selected from hydroxy, halogen, alkoxy, alkyl, haloalkyl, amino, alkylamino and cyano;

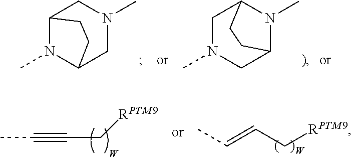

wherein W is 0, 1, or 2, and R^{PTM9} is selected from a H, alkyl (e.g., methyl), methoxy, optionally substituted 5- or 6-membered aryl, optionally substituted 5- or 6-membered heteroaryl (e.g.,

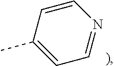

or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from a H, 5- or 6-membered heteroaryl (e.g.,

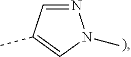

an optionally substituted 4-8-membered heterocycloalkyl (e.g. a 4-8 membered heterocycloalkyl substituted with 0, 1, or 2 substituents selected from hydroxy, halogen, alkoxy, alkyl, haloalkyl, amino, alkylamino and cyano; or

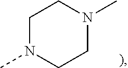

optionally substituted 8-10 membered bridged biheterocyclyl rings (e.g. a 8-10 membered bridged bicycloalkyl or bridged biheterocyclyl rings substituted with 0, 1, or 2 substituents selected from hydroxy, halogen, alkoxy, alkyl, haloalkyl, amino, alkylamino and cyano;

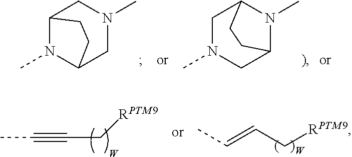

wherein W is 0, 1, or 2, and R^{PTM9} is selected from a H, alkyl (e.g., methyl), methoxy, optionally substituted 5- or 6-membered heteroaryl (e.g.,

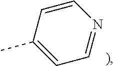

or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from a H, 5-membered heteroaryl (e.g.,

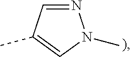

a 6-membered heterocycloalkyl substituted with 0, 1, or 2 substituents selected from C₁₋₃ alkyl; or

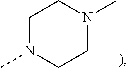

optionally substituted 9-membered bridged biheterocyclyl rings (e.g. a 9-membered bridged bicycloalkyl or bridged biheterocyclyl rings substituted with 0, 1, or 2 substituents selected from C₁₋₃ alkyl;

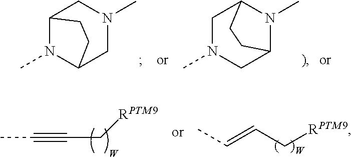

wherein W is 0, 1, or 2, and R^{PTM9} is selected from a H, alkyl (e.g., methyl), methoxy, optionally substituted 5- or 6-membered heteroaryl (e.g.,

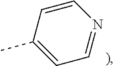

or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from a H, 5-membered heteroaryl (e.g.,

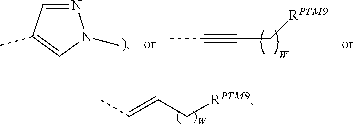

wherein W is 0, 1, or 2, and R^{PTM9} is selected from a H, alkyl (e.g., methyl), methoxy, optionally substituted 5- or 6-membered heteroaryl (e.g.,

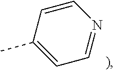

or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from a H, 5-membered heteroaryl (e.g.,

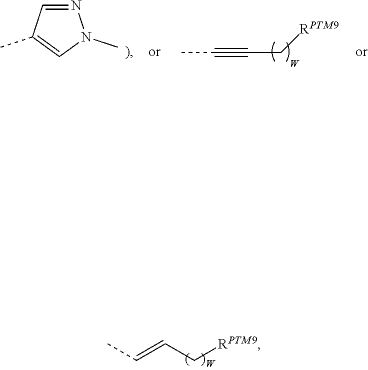

wherein W is 0, 1, or 2, and R^{PTM9} is selected from a H, alkyl (e.g., methyl), methoxy, optionally substituted 5- or 6-membered heteroaryl (e.g.,

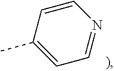

or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from H,

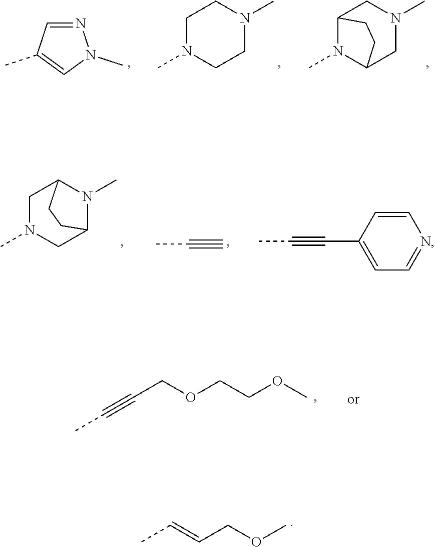

In any aspect or embodiment described herein, one of R^{PTM5} and R^{PTM6} is H, and the other is selected from H,

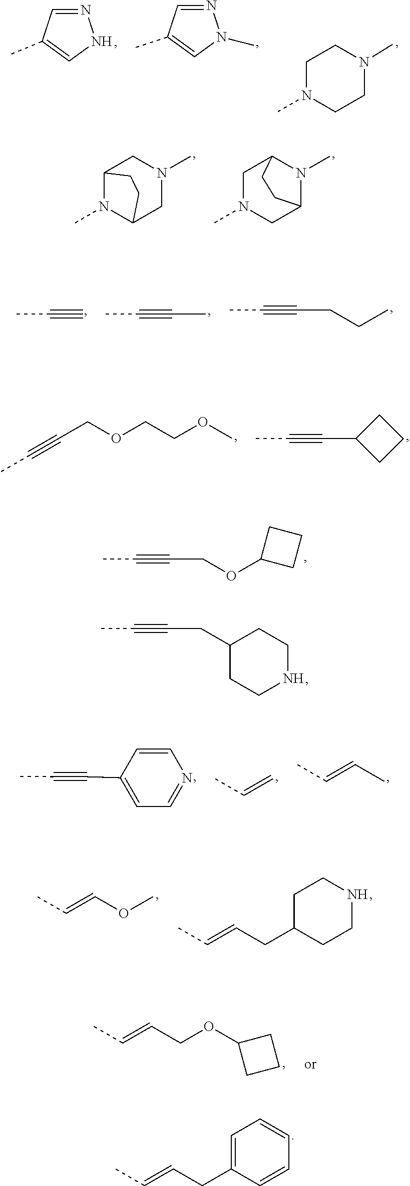

In any aspect or embodiment described herein, the BRM protein binding compound is selected from

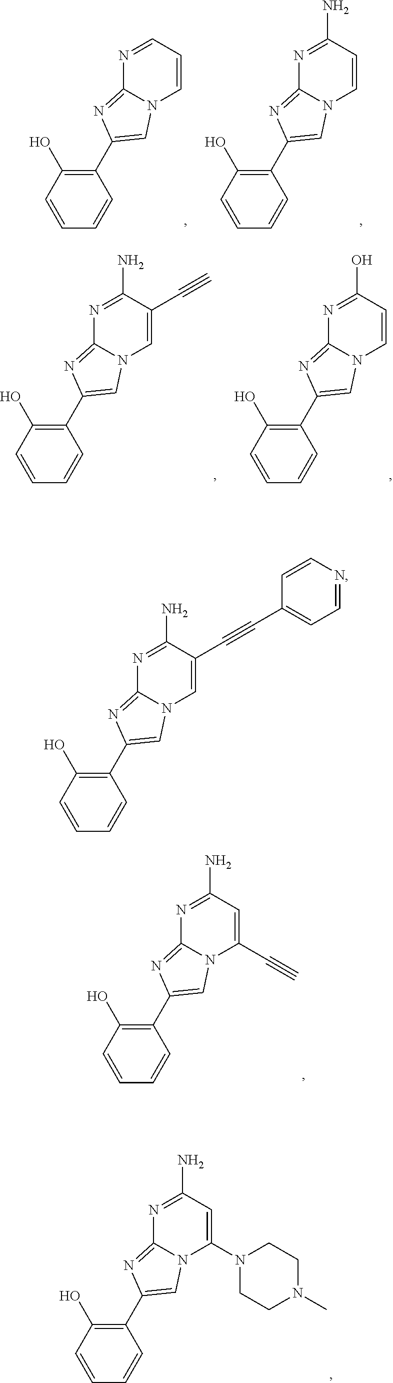

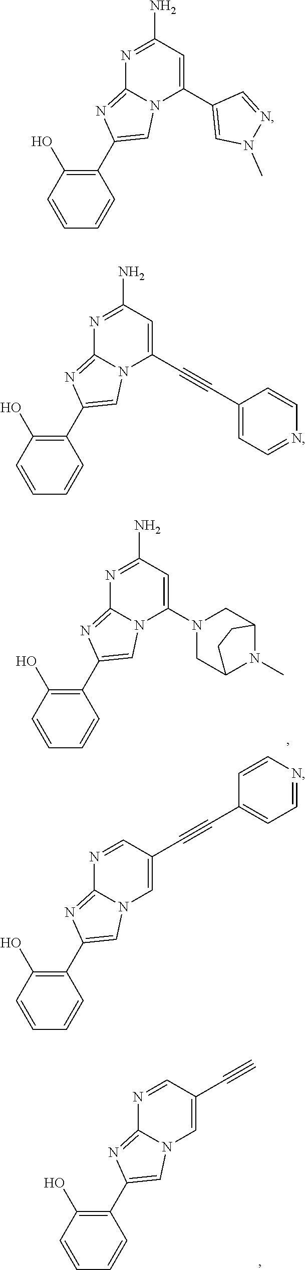 [SMILES: Cn1cc(-c2cc(N)nc3nc(-c4ccccc4O)cn23)cn1 ; Nc1cc(C#Cc2ccncc2)n2cc(-c3ccccc3O)nc2n1 ; CN1C2CCC1CN(c1cc(N)nc3nc(-c4ccccc4O)cn13)C2 ; Oc1ccccc1-c1cn2cc(C#Cc3ccncc3)cnc2n1 ; C#Cc1cnc2nc(-c3ccccc3O)cn2c1]

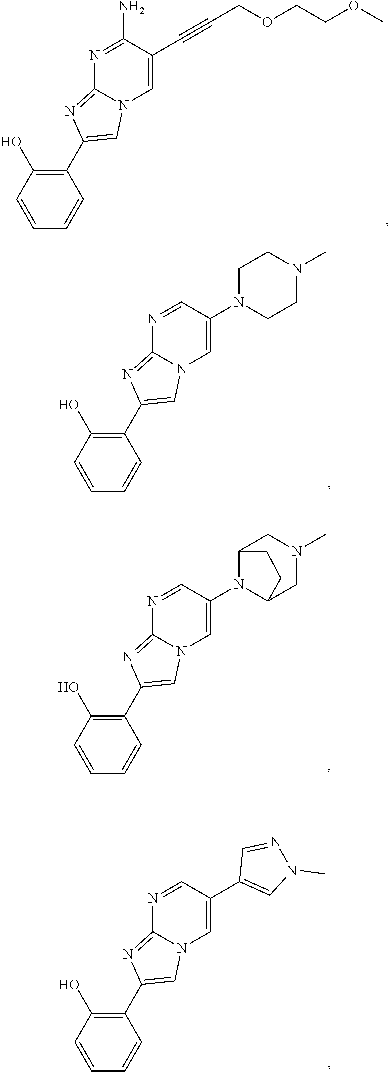 [SMILES: COCCOCC#Cc1cn2cc(-c3ccccc3O)nc2nc1N ; CN1CCN(c2cnc3nc(-c4ccccc4O)cn3c2)CC1 ; CN1CC2CCC(C1)N2c1cnc2nc(-c3ccccc3O)cn2c1 ; Cn1cc(-c2cnc3nc(-c4ccccc4O)cn3c2)cn1]

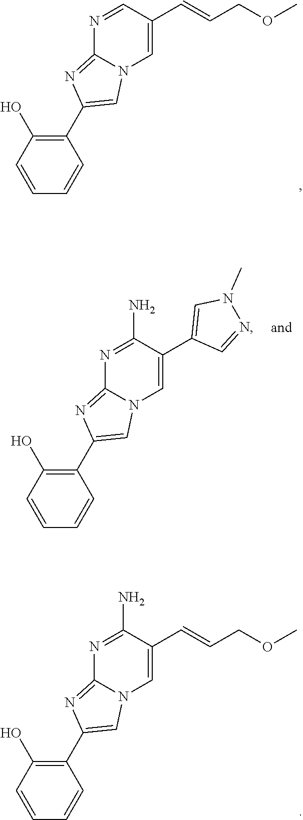 [SMILES: COC/C=C/c1cnc2nc(-c3ccccc3O)cn2c1 ; Cn1cc(-c2cn3cc(-c4ccccc4O)nc3nc2N)cn1 ; COC/C=C/c1cn2cc(-c3ccccc3O)nc2nc1N]

### Bifunctional Compounds

Disclosed herein are bifunctional compounds that function to recruit endogenous proteins to an E3 ubiquitin ligase for degradation and methods of using the same. In particular, disclosed herein are bifunctional or proteolysis targeting chimeric (PROTAC®) compounds that are modulators of targeted ubiquitination of a variety of polypeptides and other proteins, which then are degraded and/or otherwise inhibited by the bifunctional compounds.

Other bifunctional or proteolysis targeting chimeric (PROTAC) compounds that are modulators of targeted ubiquitination are disclosed in U.S. Nonprovisional patent application Ser. No. 16/590,329, filed 1 Oct. 2020, published as U.S. Patent Application Publication No. 2020/0038378 A1; U.S. Nonprovisional application Ser. No. 16/372,345, filed 1 Apr. 2019, published as U.S. Patent Application Publication No. 2019/0300521 A1; U.S. Provisional Patent Application Ser. No. 62/651,186, filed: 1 Apr. 2018, titled BRM TARGETING PROTAC COMPOUNDS AND ASSOCIATED METHODS OF USE; and U.S. Provisional Patent Application Ser. No. 62/797,754, filed: 28 Jan. 2019, titled BRM TARGETING PROTAC COMPOUNDS AND ASSOCIATED METHODS OF USE; U.S. patent application Ser. No. 15/230,354, filed on Aug. 5, 2016; and U.S. patent application Ser. No. 14/371,956, filed on Jul. 11, 2014, published as U.S. Patent Application Publication No. 2014/0356322; and U.S. patent application Ser. No. 15/074,820, filed on Mar. 18, 2016, published as U.S. Patent Application Publication No. 2016/0272639; and International Patent Application No. PCT/US2016/019328, filed Feb. 24, 2016, published as International Patent Application Publication No. WO2016/138114, and International Patent Application No. PCT/US2016/023258, filed Mar. 18, 2016, published as International Patent Application Publication No. WO2016/149668, and U.S. Non-Provisional patent application Ser. No. 15/885,671, filed 31 Jan. 2018, published as U.S. Patent Application Publication No. 2018/0215731, all of which are incorporated herein by reference in their entirety.

The disclosed bifunctional compounds provide a broad range of advantageous pharmacological activities that are consistent with the degradation/inhibition of targeted polypeptides from a multitude of different protein classes and/or families.

In one aspect, the description provides compounds comprising an E3 ubiquitin ligase binding moiety (“ULM”) that is a Von Hippel-Lindae E3 ubiquitin ligase (VHL) binding moiety (VLM). In an exemplary embodiment, the ULM is coupled to a target protein binding moiety (PTM) via a chemical linker (L) according to the structure:

PTM-L-ULM (A)

wherein L is a bond or a chemical linker group, ULM is a E3 ubiquitin ligase binding moiety, and PTM is a target protein binding moiety. The number and/or relative positions of the moieties in the compounds illustrated herein is provided by way of example only. As would be understood by the skilled artisan, compounds described herein can be synthesized with any desired number and/or relative position of the respective functional moieties.

In another aspect, the present disclosure provides bifunctional or multifunctional compounds useful for regulating protein activity by inducing the degradation of a target protein. In certain embodiments, the compound comprises a VLM coupled, e.g., linked covalently, directly or indirectly, to a moiety that binds a target protein (i.e., a protein targeting moiety or a “PTM”). In certain embodiments, the VLM and PTM are joined or coupled via a chemical linker (L). The VLM binds VHL, and the PTM recognizes a target protein and the interaction of the respective moieties with their targets facilitates the degradation of the target protein by placing the target protein in proximity to the ubiquitin ligase protein. An exemplary bifunctional compound can be depicted as: PTM-VLM.

In certain embodiments, the bifunctional compound further comprises a chemical linker (“L”). For example, the bifunctional compound can be depicted as: PTM-L-VLM, wherein the PTM is a protein/polypeptide targeting moiety, the L is a chemical linker, and the VLM is a VHL binding moiety.

In certain embodiments, the ULM (e.g., VLM) shows activity or binds to the E3 ubiquitin ligase (e.g., VHL) with an IC₅₀ of less than about 200 μM. The IC₅₀ can be determined according to any method known in the art, e.g., a fluorescent polarization assay.

In certain additional embodiments, the bifunctional compounds described herein demonstrate an activity with an IC₅₀ of less than about 100, 50, 10, 1, 0.5, 0.1, 0.05, 0.01, 0.005, 0.001 mM, or less than about 100, 50, 10, 1, 0.5, 0.1, 0.05, 0.01, 0.005, 0.001 μM, or less than about 100, 50, 10, 1, 0.5, 0.1, 0.05, 00.01, 0.005, 0.001 nM, or less than about 100, 50, 10, 1, 0.5, 0.1, 0.05, 0.01, 0.005, 0.001 pM.

In any aspect or embodiment described herein, the bifunctional compound includes compounds having a DC₅₀ of <about 2.5 nM (i.e., category A as described herein), wherein the DC₅₀ is optionally determined as described herein.

In any aspect or embodiment described herein, the bifunctional compound includes compounds having a DC₅₀ that is ≥about 2.5 nM and <about 10 nM (i.e., category B as described herein), wherein the DC₅₀ is optionally determined as described herein.

In any aspect or embodiment described herein, the bifunctional compound includes compounds having a DC₅₀ of ≥about 2.5 nM and <about 30 nM (i.e., category C as described herein), wherein the DC₅₀ is optionally determined as described herein.

In any aspect or embodiment described herein, the bifunctional compound includes compounds having a DC₅₀ of ≥about 30 nM (i.e., category D as described herein), wherein the DC₅₀ is optionally determined as described herein.

In any aspect or embodiment described herein, a compound or compounds having a DC₅₀ of ≥about 30 nM (i.e., category D as described herein) is or are excluded (optionally, the DC₅₀ can be determined as described herein).

In any aspect or embodiment described herein the DC₅₀ value of the bifunctional compounds described herein can be determined according to any method know in the art, such as, for example, a fluorescent polarization assay or as described herein.

In any aspect or embodiment described herein, the bifunctional compound includes compounds having a D_{Max} of >about 75% degraded (i.e., category A as described herein), wherein the D_{Max} is optionally determined as described herein.

In any aspect or embodiment described herein, the bifunctional compound includes compounds having a D_{Max} that is >about 50% degraded and about 75% degraded (i.e., category B as described herein), wherein the DC₅₀ is optionally determined as described herein.

In any aspect or embodiment described herein, the bifunctional compound includes compounds having a D_{Max} of >about 50% degraded (i.e., category C as described herein), wherein the D_{Max} is optionally determined as described herein.

In any aspect or embodiment described herein a compound or compounds having a D_{Max} of ≤about 50% degraded (i.e., category C as described herein) is or are excluded (optionally, the D_{Max} can be determined as described herein).

In any aspect or embodiment described herein the D_{Max} value of the bifunctional compounds described herein can be determined according to any method know in the art, such as, for example, a fluorescent polarization assay or as described herein.

In certain embodiments, where the compound comprises multiple ULMs, the ULMs are identical. In additional embodiments, the compound comprising a plurality of ULMs (e.g., ULM, ULM′, etc.), at least one PTM coupled to a ULM directly or via a chemical linker (L) or both. In certain additional embodiments, the compound comprising a plurality of ULMs further comprises multiple PTMs. In still additional embodiments, the PTMs are the same or, optionally, different. In still further embodiments, wherein the PTMs are different, the respective PTMs may bind the same protein target or bind specifically to a different protein target.

In certain embodiments, the compound may comprise a plurality of ULMs and/or a plurality of ULM's. In further embodiments, the compound comprising at least two different ULMs, a plurality of ULMs, and/or a plurality of ULM's further comprises at least one PTM coupled to a ULM or a ULM′ directly or via a chemical linker or both. In any of the embodiments described herein, a compound comprising at least two different ULMs can further comprise multiple PTMs. In still additional embodiments, the PTMs are the same or, optionally, different. In still further embodiments, wherein the PTMs are different the respective PTMs may bind the same protein target or bind specifically to a different protein target.

Any aspect or embodiment described herein, the compound has a chemical structure selected from:

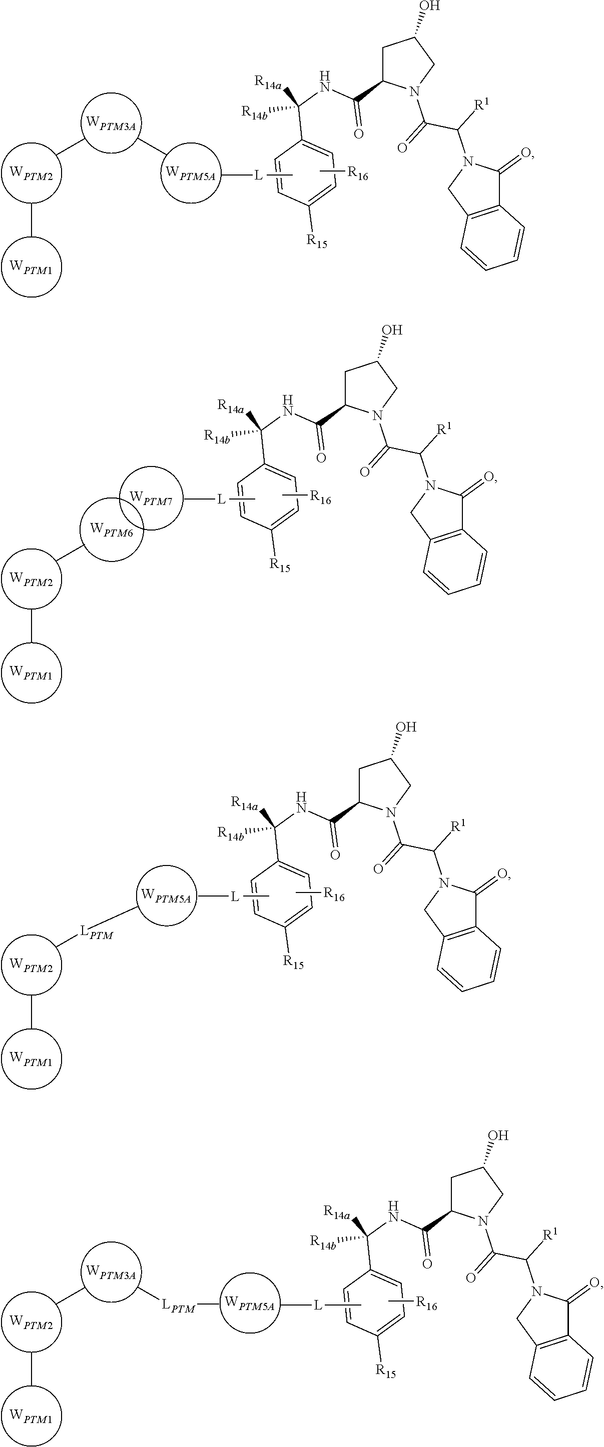

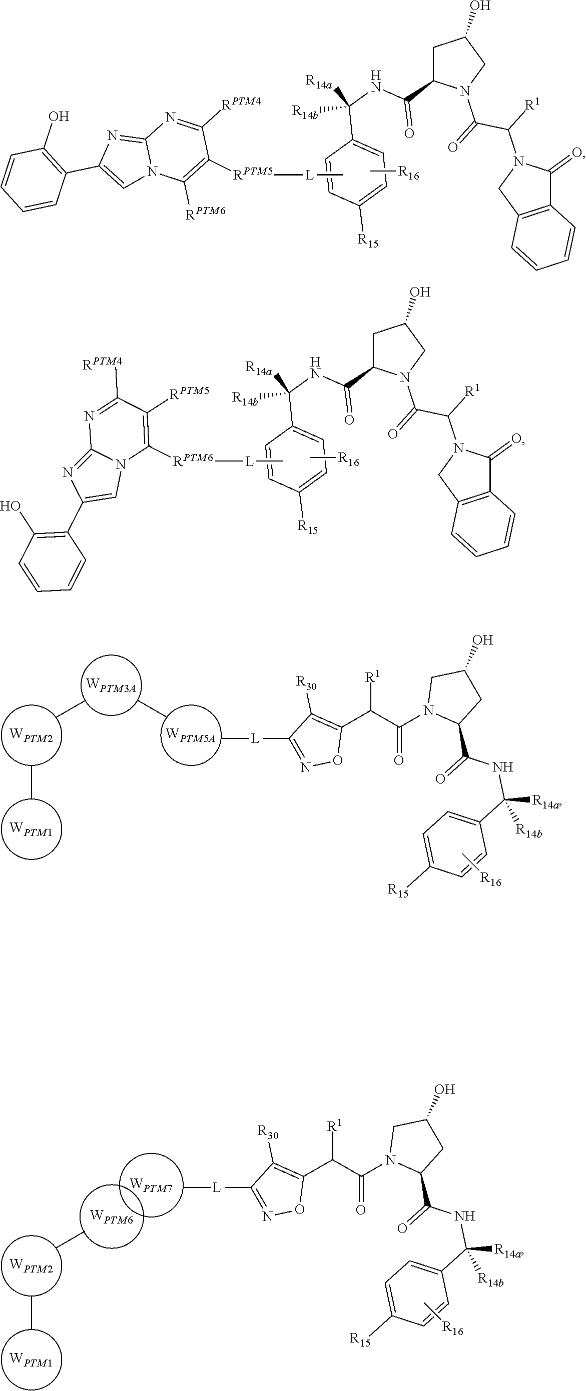

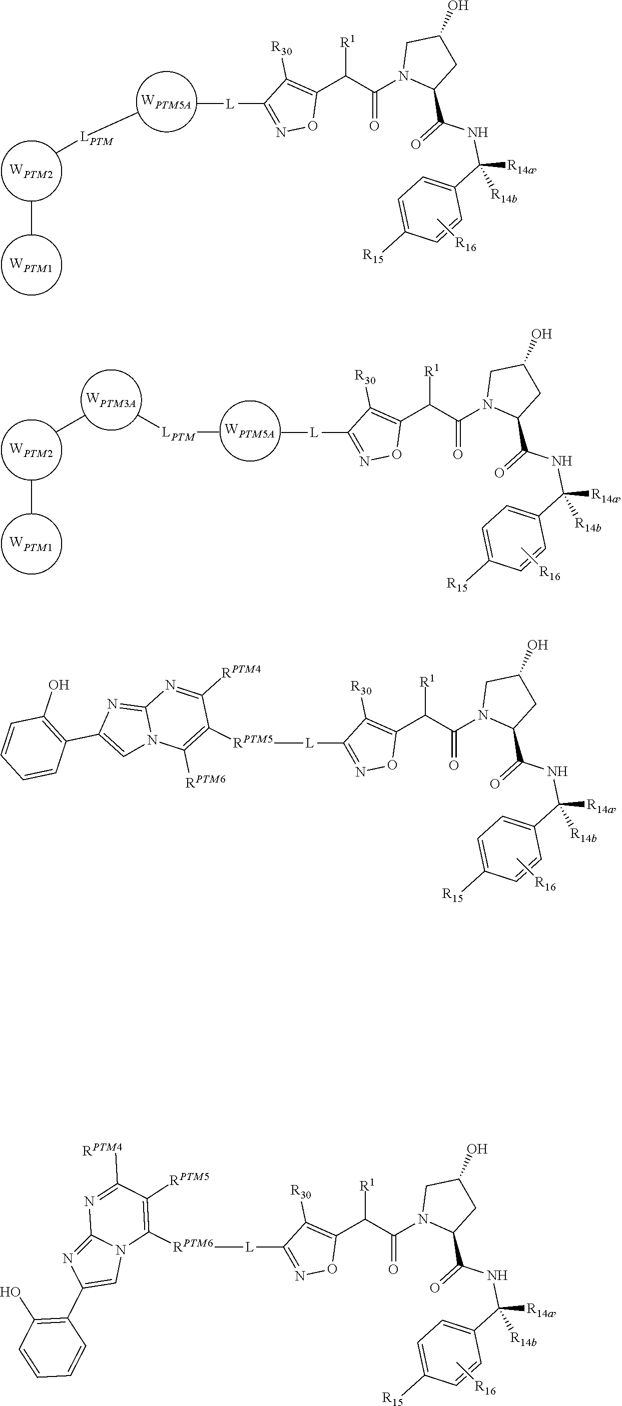

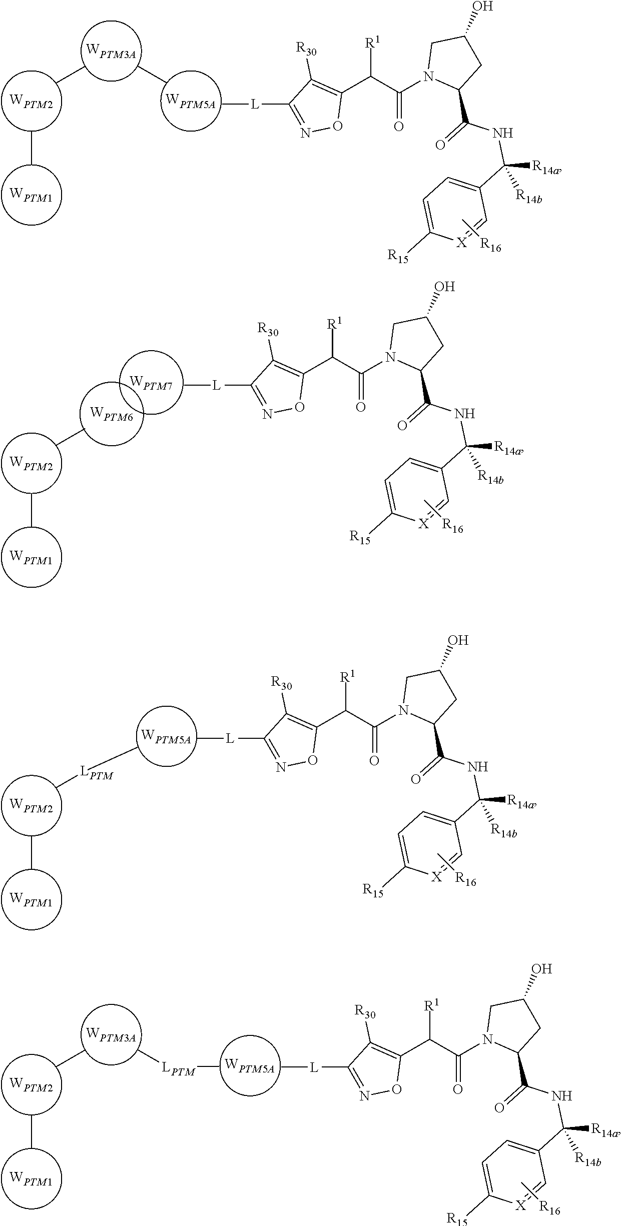

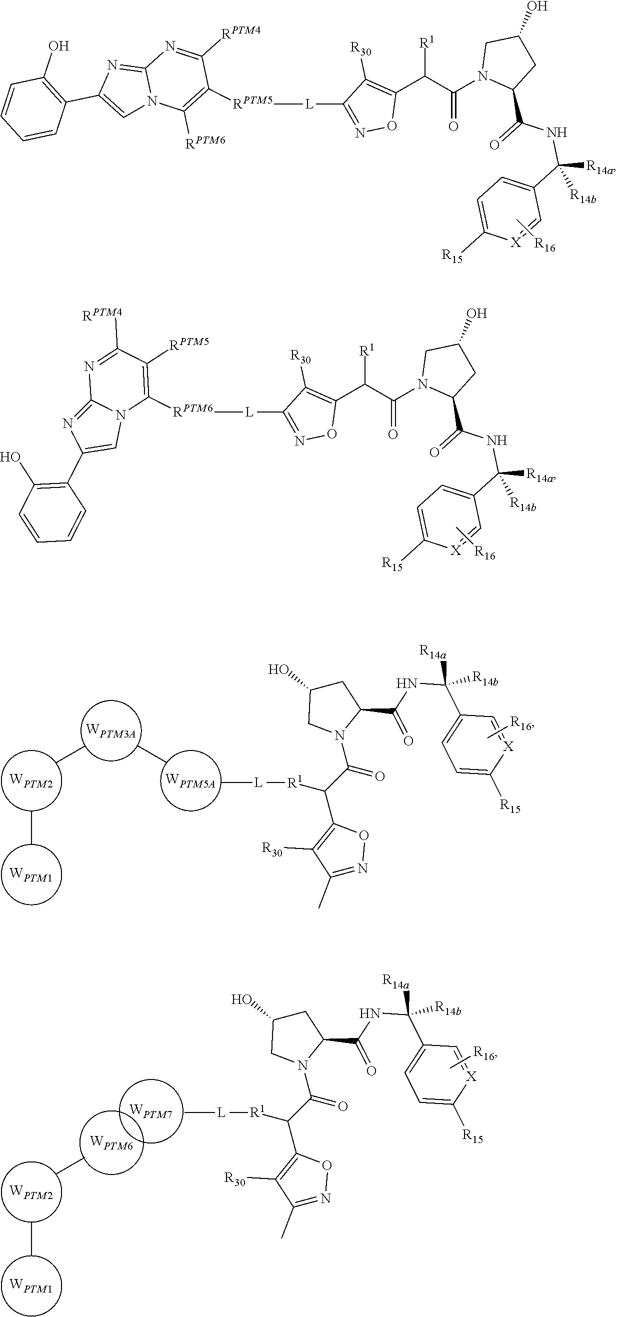

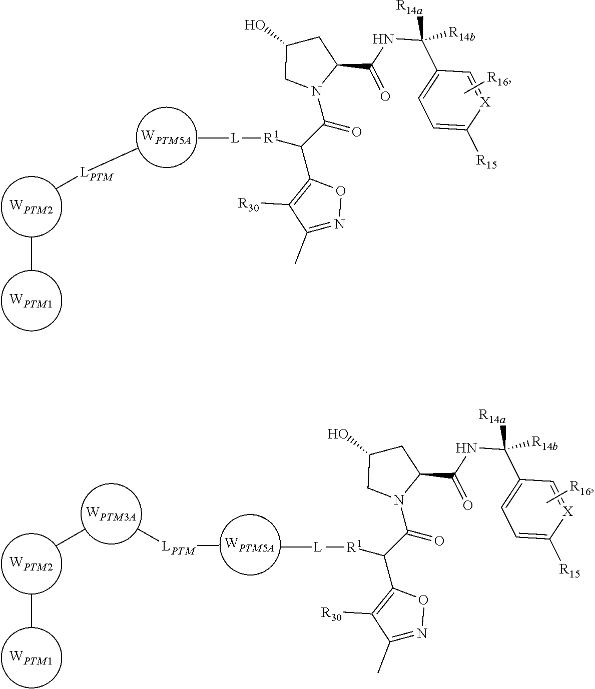

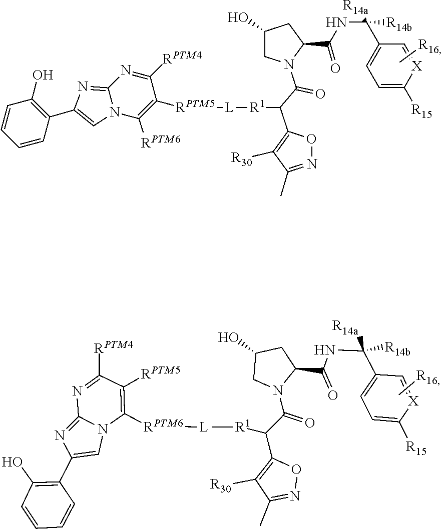

or a pharmaceutically acceptable salt thereof,
   wherein W_{PTM1}, W_{PTM2}, W_{PTM3A}, W_{PTM6}, W_{PTM7}, W_{PTM5A}, W_{PTM3B}, R^{PTM4}, R^{PTM5}, R^{PTM6}, X, R₃₀, R₁, R_{28A}, R_{28B}, R₂₈, R₁₄ₐ, R_{14b}, R₁₅, and R₁₆ are as defined in any aspect or embodiment described herein.

Any aspect or embodiment described herein, the compound has a chemical structure selected from:

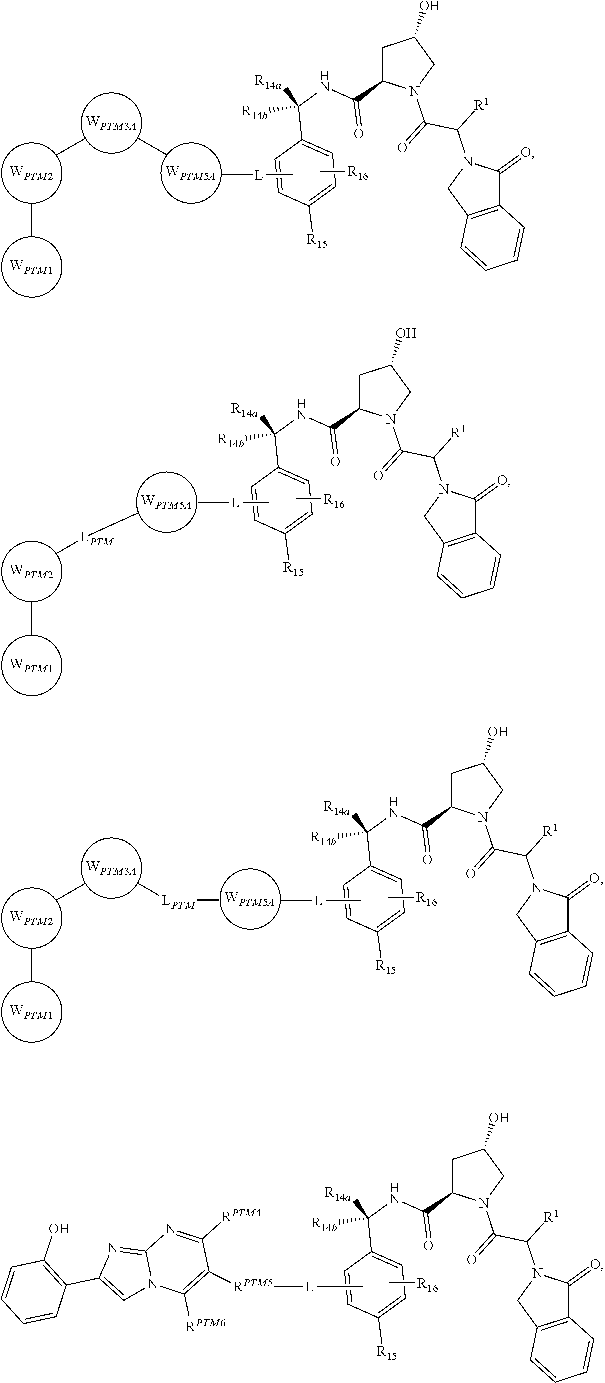

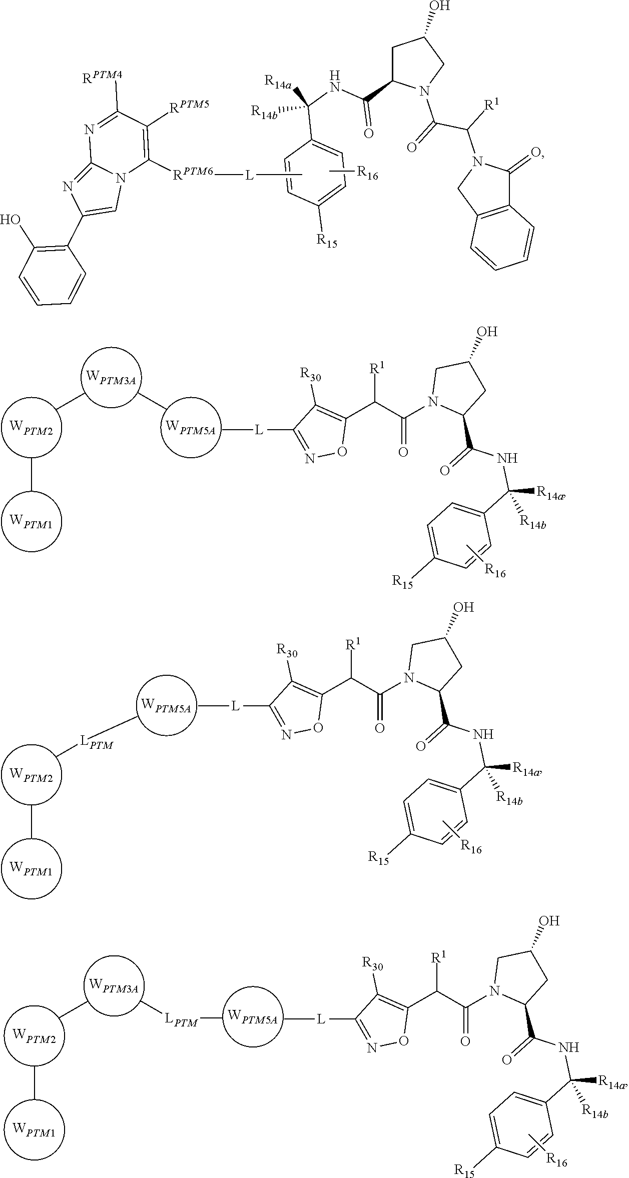

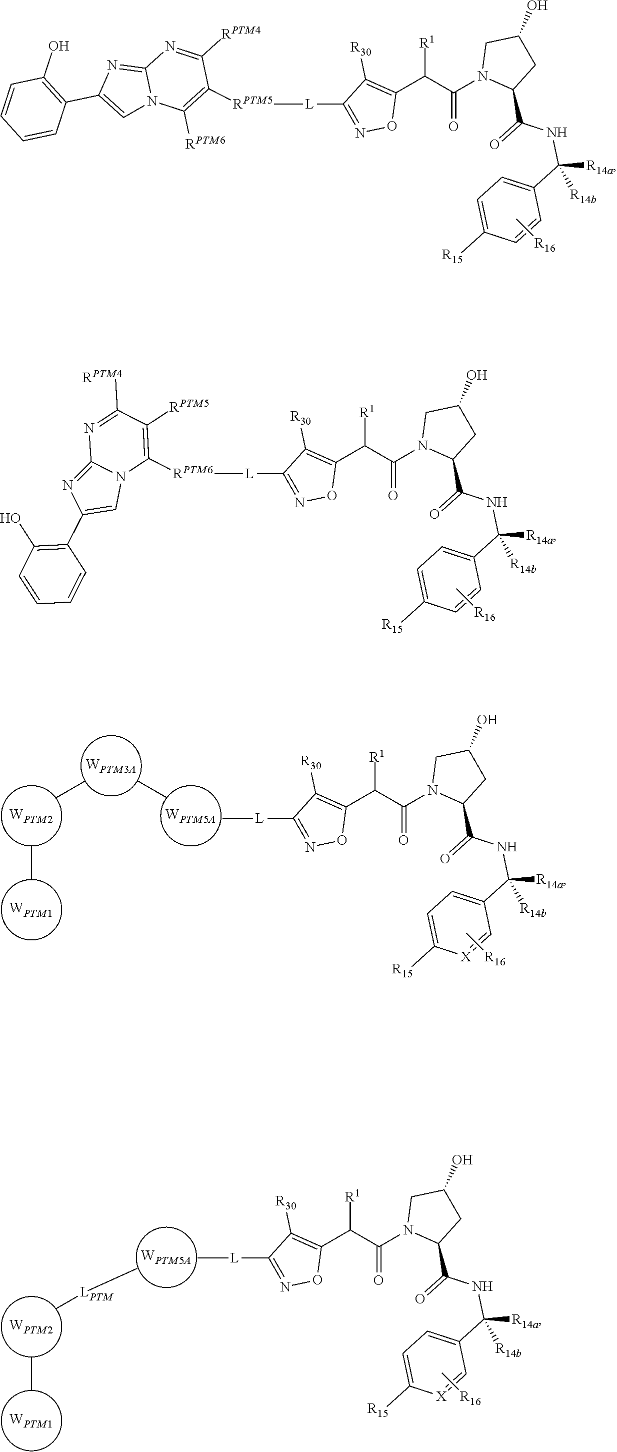

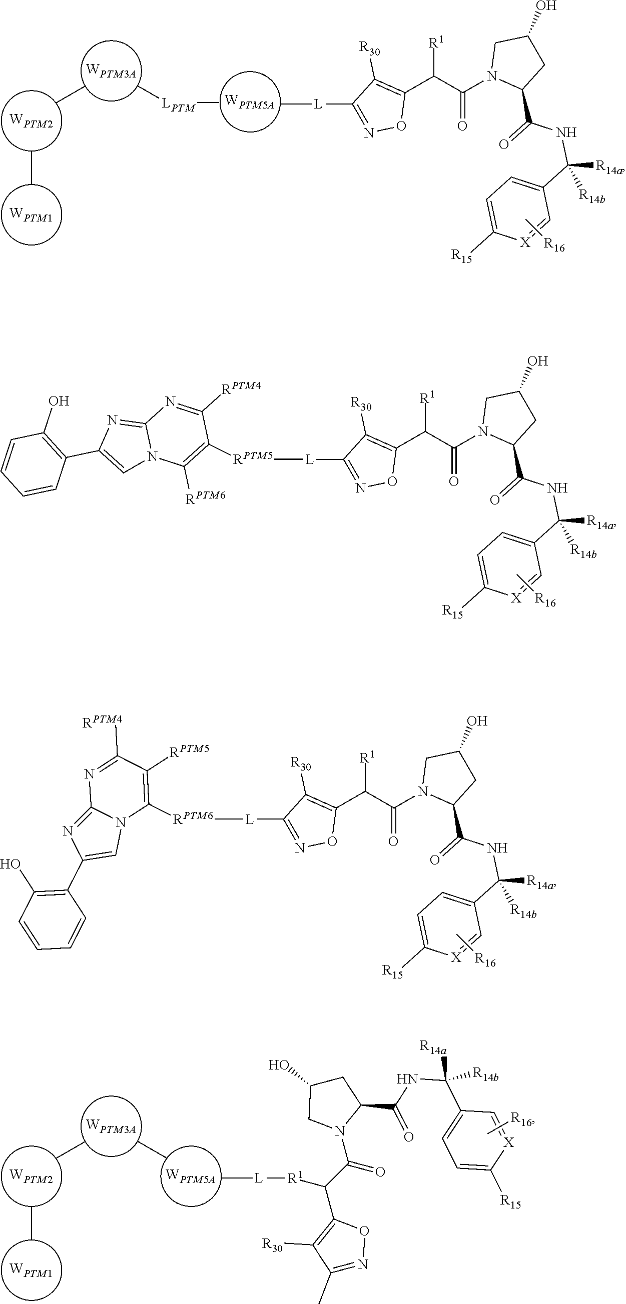

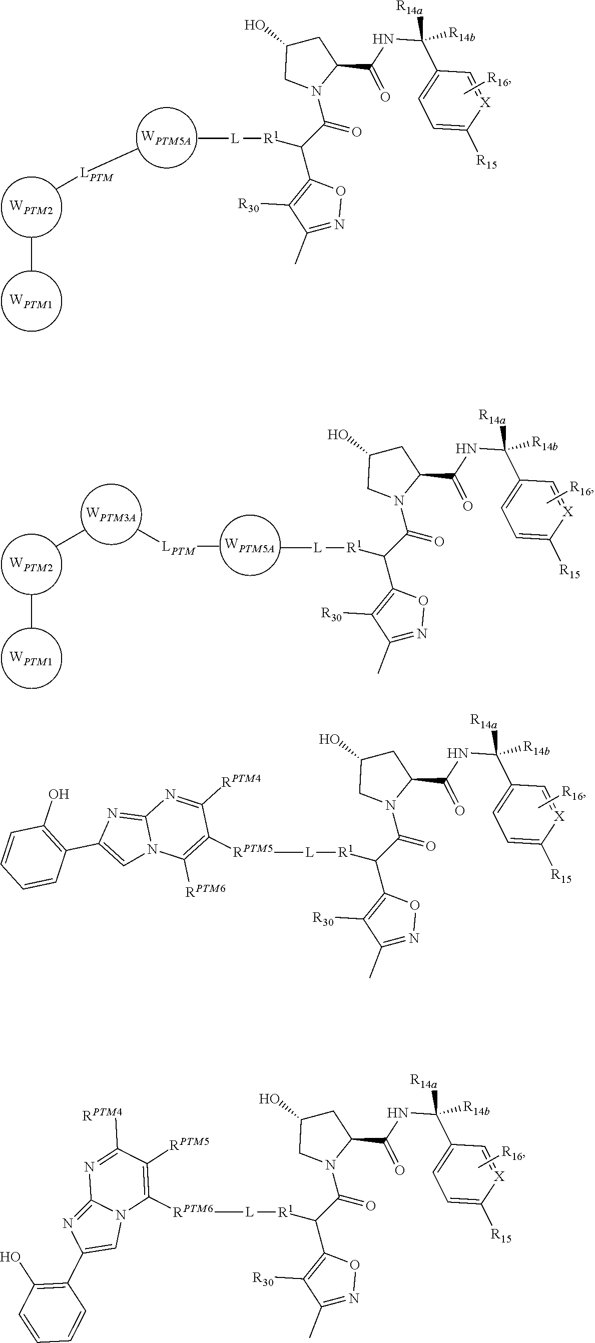

or a pharmaceutically acceptable salt thereof,
   wherein:
W_{PTM1}, W_{PTM2}, W_{PTM3A}, W_{PTM6}, W_{PTM7}, W_{PTM5A}, W_{PTM3B}, R^{PTM4}, R^{PTM5}, R^{PTM6}, R₁₄ₐ, R_{14b}, R₁₅, and R₁₆ are as defined in any aspect or embodiment described herein;
X is CH or N;
R₃₀ is H, F or Cl; and
R₁ is a C₁₋₆ alkyl.

Any aspect or embodiment described herein, the compound has a chemical structure selected from:

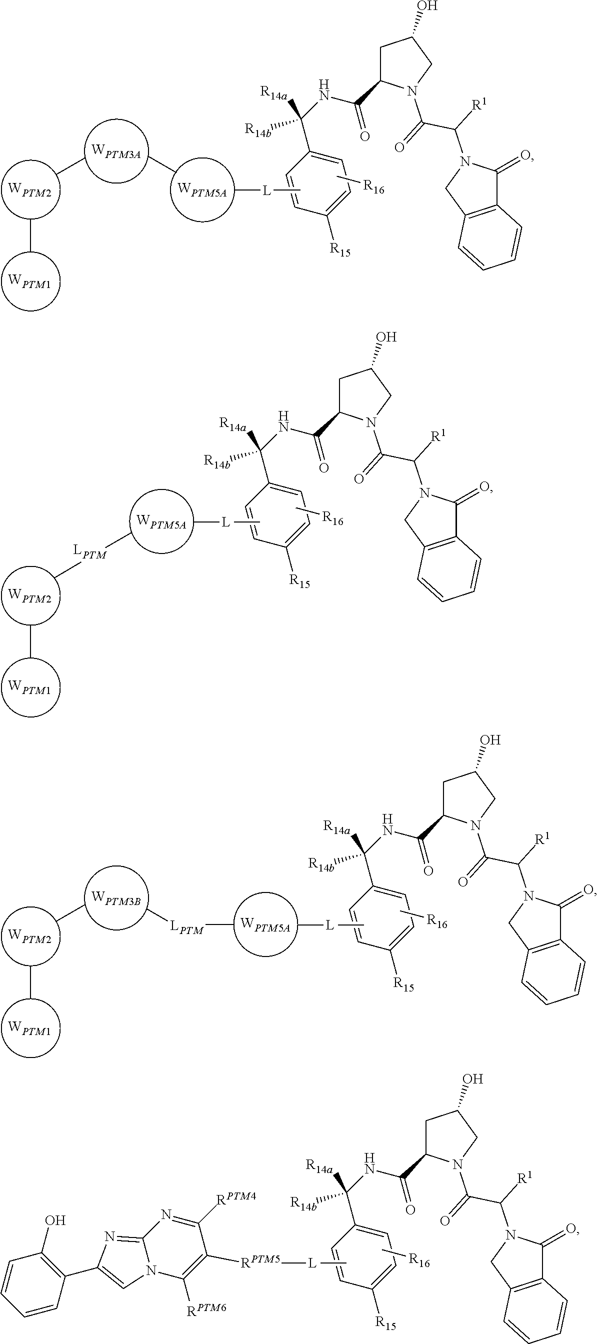

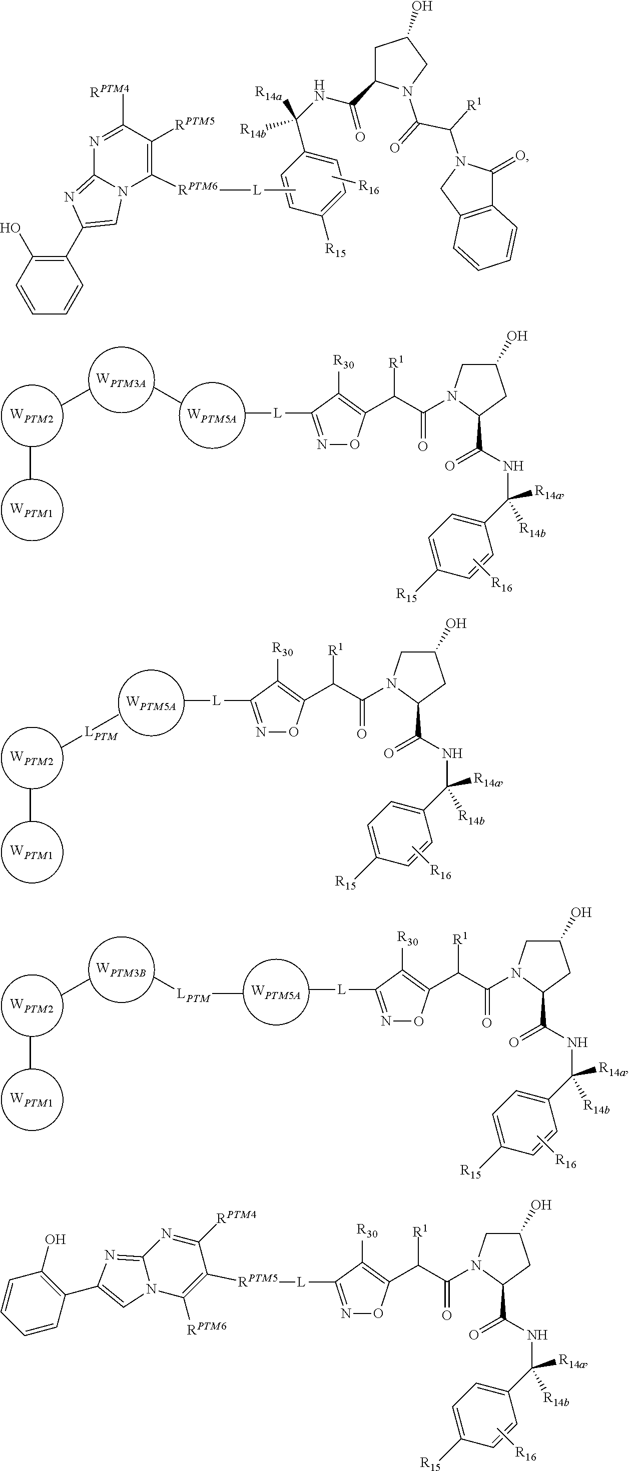

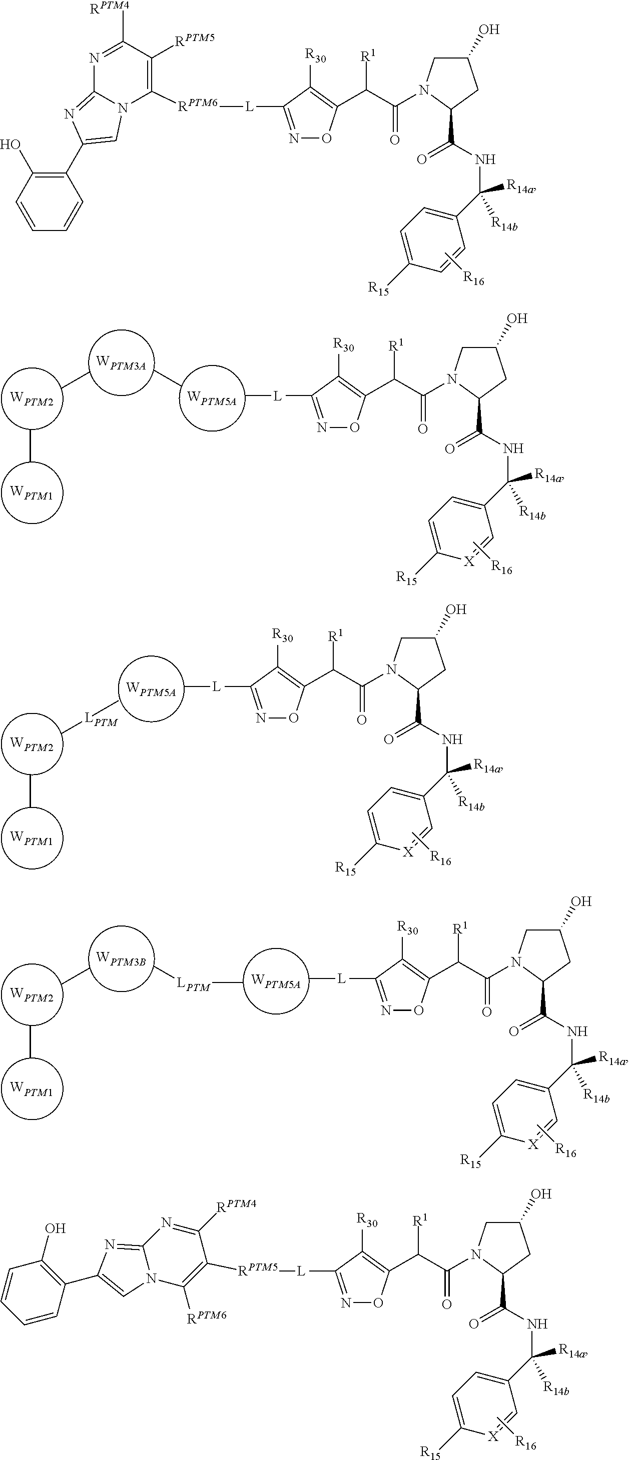

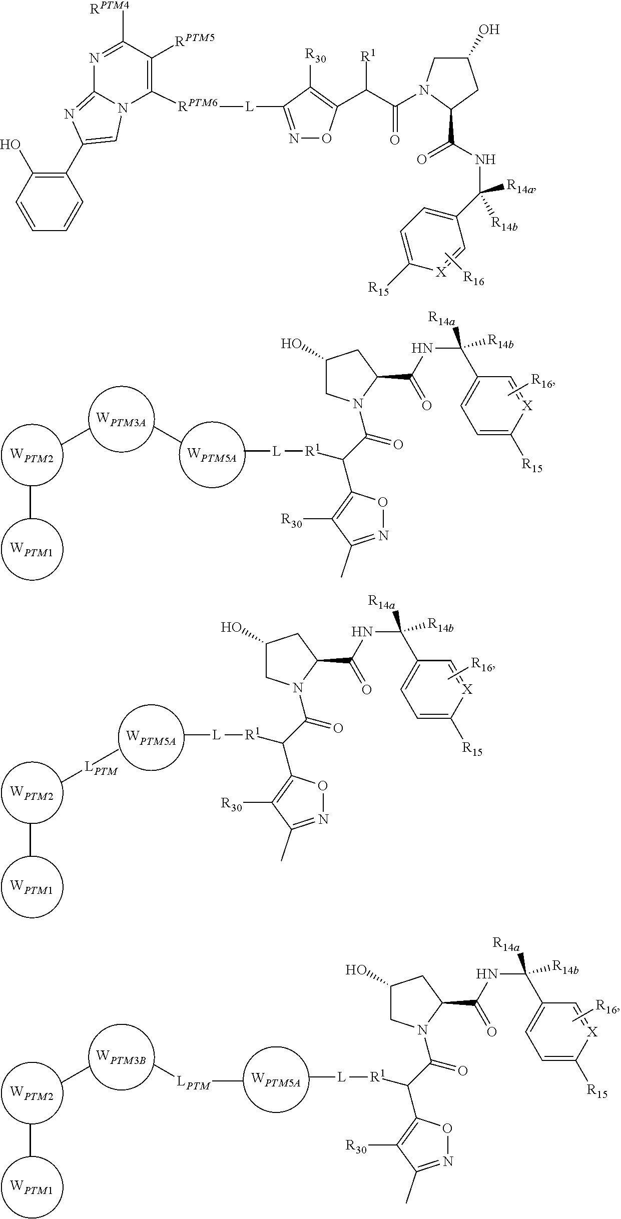

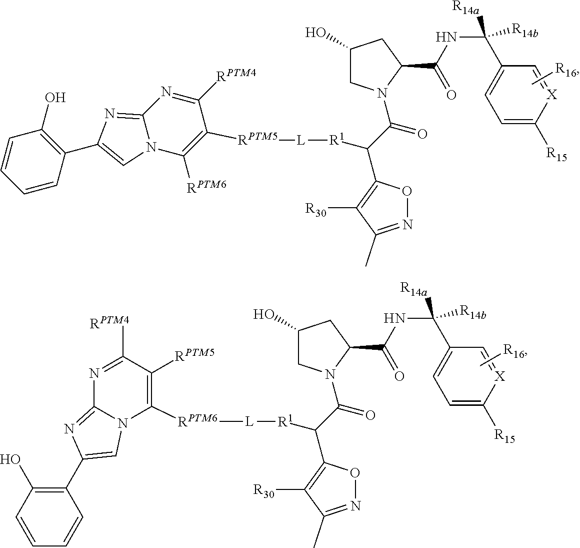

or a pharmaceutically acceptable salt thereof,
   wherein:
W_{PTM1}, W_{PTM2}, W_{PTM3A}, W_{PTM6}, W_{PTM7}, W_{PTM5A}, W_{PTM3B}, R^{PTM4}, R^{PTM5}, and R^{PTM6} are as defined in any aspect or embodiment described herein;
X is CH or N;
R₃₀ is H, F or Cl;
R₁ is a C₁₋₆ alkyl;
R₁₅ is selected from: cyano, halogen (e.g., F or Cl),

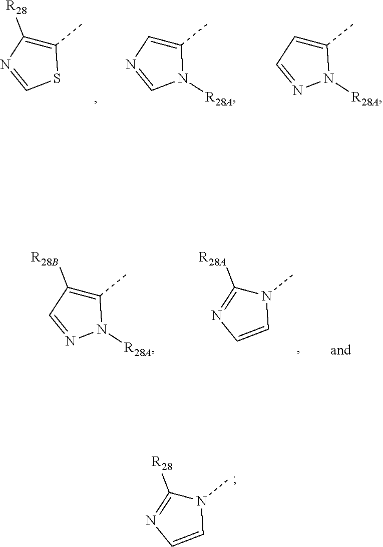

and
R¹⁶ is one or two groups individually selected from H, C₁₋₄alkyl, fluoro, chloro, NH₂, CN, or C₁₋₄alkoxy;
R_{28A} is selected from H or methyl;
R_{28B} is selected from H, methyl, and halogen (e.g., F or Cl);
R₂₈ is H, methyl, CH₂N(Me)₂, CH₂OH, CH₂O(C₁₋₄alkyl), CH₂NHC(O)C₁₋₄alkyl, NH₂,

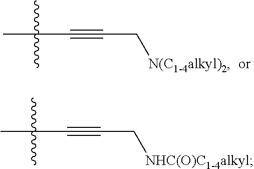

and
one of R₁₄ₐ and R_{14b} is a H, methyl, C1 fluoroalkyl, CHF₂, CF₃, and the other is a H.

In additional embodiments, the description provides the compounds as described herein including their enantiomers, diastereomers, solvates and polymorphs, including pharmaceutically acceptable salt forms thereof, e.g., acid and base salt forms.

### Exemplary VLMs

In any aspect or embodiment described herein, the ULM has a chemical structure selected from:

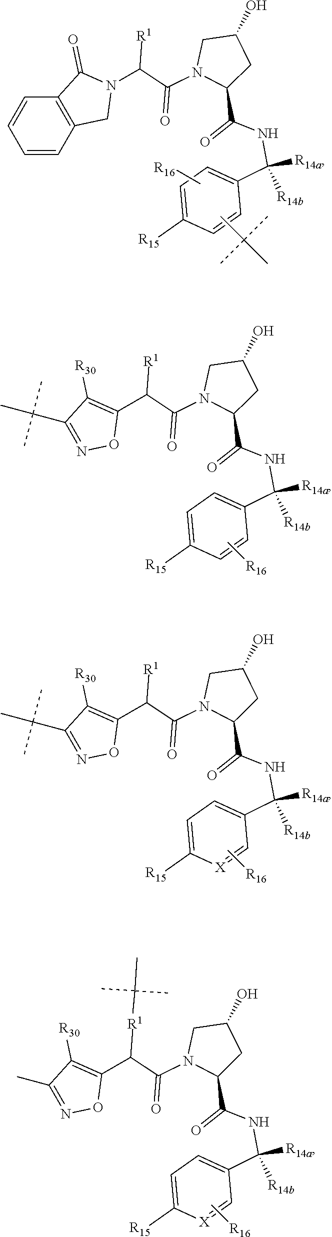

or a pharmaceutically acceptable salt thereof,
wherein X, R₃₀, R₁, R_{28A}, R_{28B}, R₂₈, R₁₄ₐ, R_{14b}, R₁₅, and R₁₆ are as defined in any aspect or embodiment described herein.

In any aspect or embodiment described herein, the ULM has a chemical structure selected from:

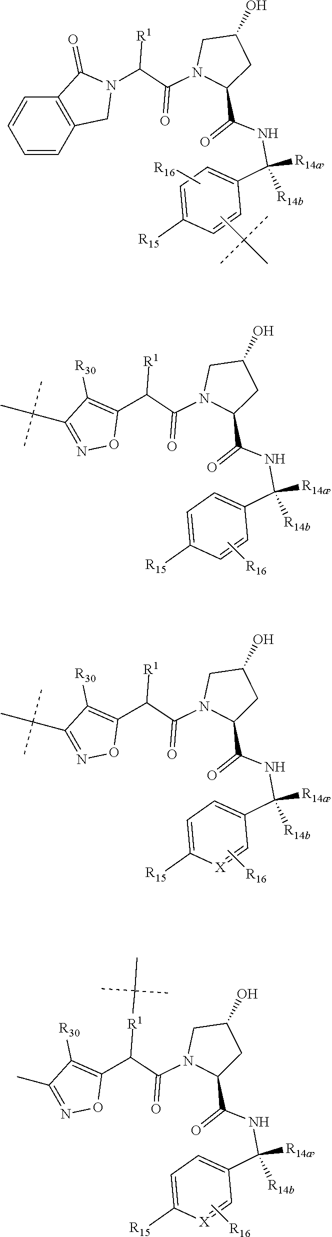

or a pharmaceutically acceptable salt thereof,
wherein:
R₁₄ₐ, R_{14b}, R₁₅, and R₁₆ are as defined in any aspect or embodiment described herein;
X is CH or N;
R₃₀ is H, F or Cl;
R₁ is a C₁₋₆ alkyl;
R_{28A} is selected from H or methyl;
R_{28B} is selected from H, methyl, and halogen (e.g., F or Cl); and
R₂₈ is H, methyl, CH₂N(Me)₂, CH₂OH, CH₂O(C₁₋₄alkyl), CH₂NHC(O)C₁₋₄alkyl, NH₂,

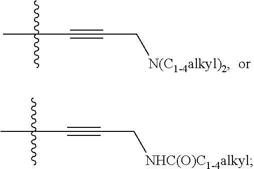

In any aspect or embodiment described herein, the ULM has a chemical structure selected from:

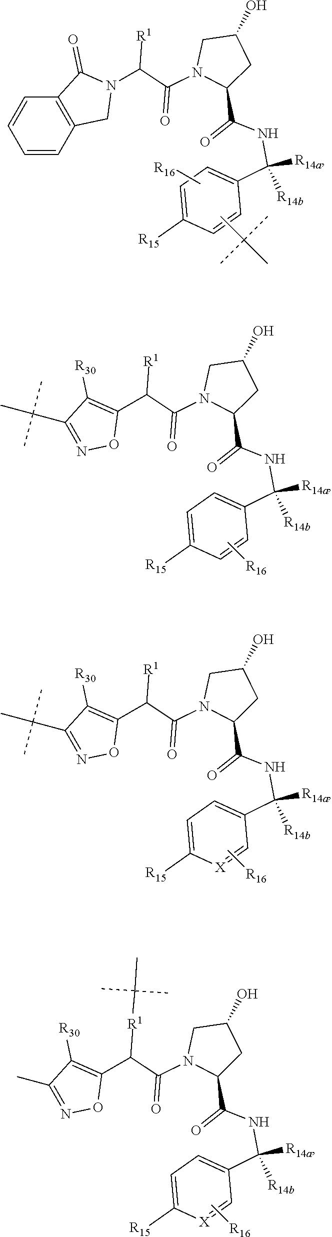

or a pharmaceutically acceptable salt thereof,
wherein:
X is CH or N;
R₃₀ is H, F or Cl;
R₁ is a C₁₋₆ alkyl;
R_{28A} is selected from H or methyl;
R_{28B} is selected from H, methyl, and halogen (e.g., F or Cl);
R₂₈ is H, methyl, CH₂N(Me)₂, CH₂OH, CH₂O(C₁₋₄alkyl), CH₂NHC(O)C₁₋₄alkyl, NH₂,

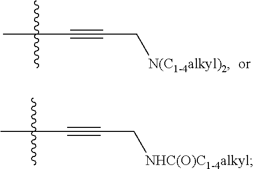

one of R₁₄ₐ and R_{14b} is a H, methyl, C₁ fluoroalkyl, CHF₂, CF₃, and the other is a H;
R₁₅ is selected from: cyano, halogen (e.g., F or Cl),

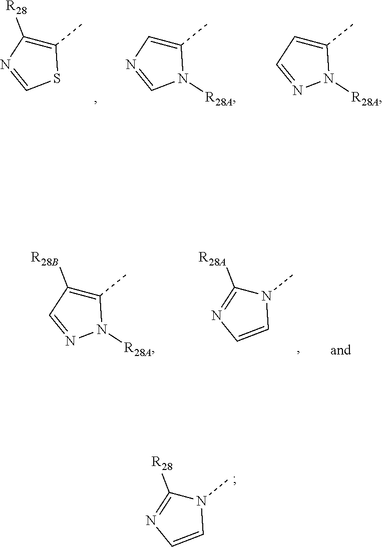

and
R¹⁶ is one or two groups individually selected from H, C₁₋₄alkyl, fluoro, chloro, NH₂, CN, or C₁₋₄alkoxy.

In any aspect or embodiment described herein, the ULM is selected from:

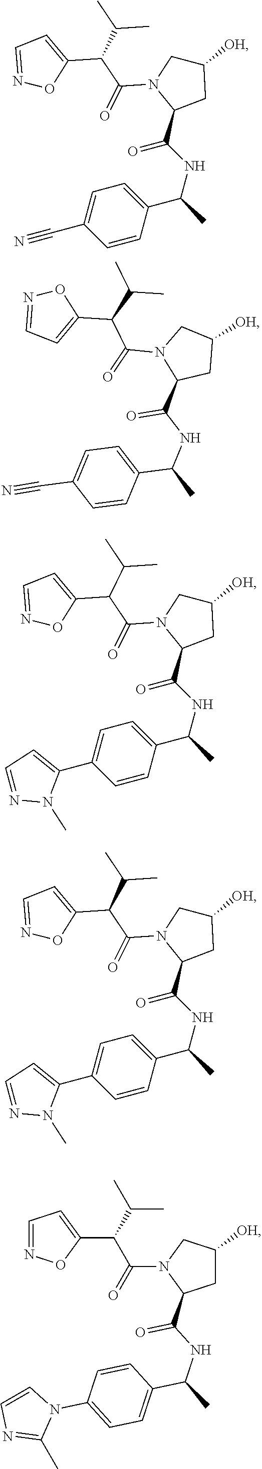

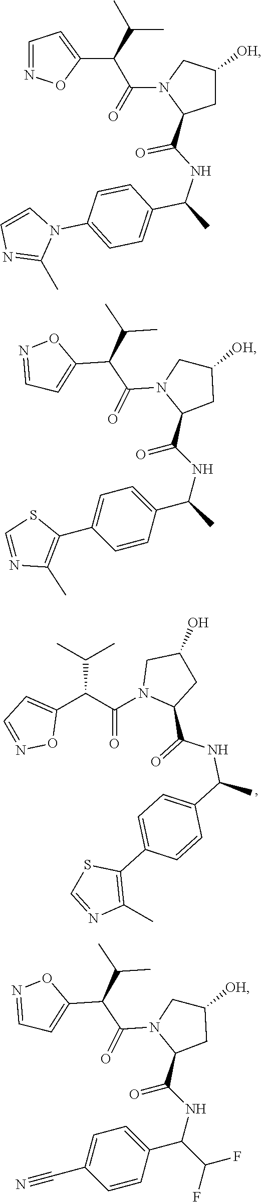

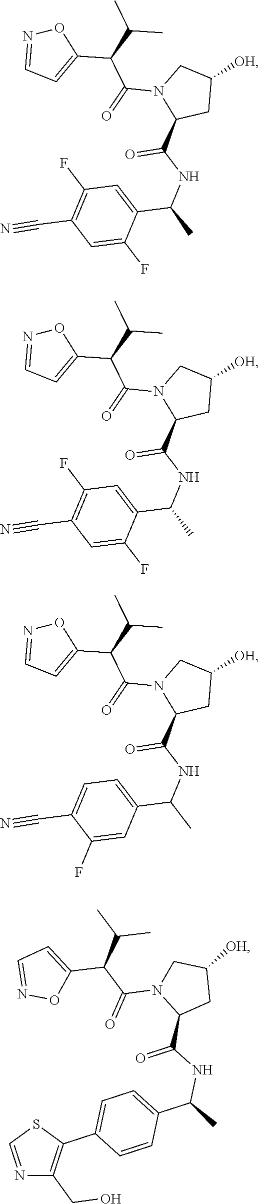

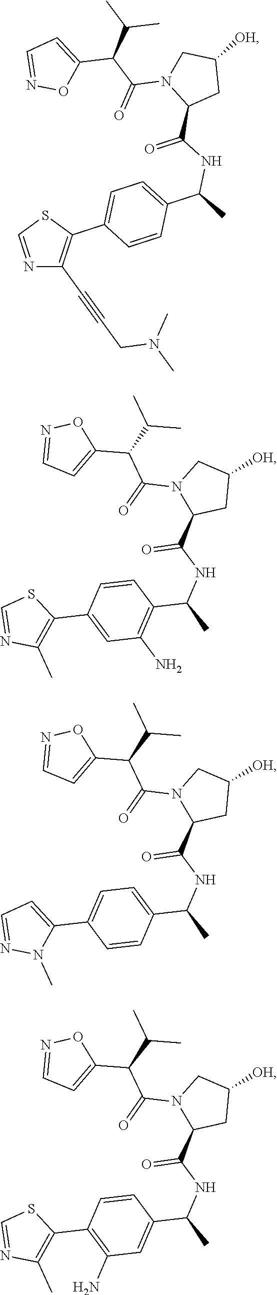 [SMILES: CC(C)[C@@H](C(=O)N1C[C@H](O)C[C@H]1C(=O)N[C@@H](C)c1ccc(-c2scnc2C#CCN(C)C)cc1)c1ccno1 ; Cc1ncsc1-c1ccc([C@H](C)NC(=O)[C@@H]2C[C@@H](O)CN2C(=O)[C@H](c2ccno2)C(C)C)c(N)c1 ; CC(C)[C@@H](C(=O)N1C[C@H](O)C[C@H]1C(=O)N[C@@H](C)c1ccc(-c2ccnn2C)cc1)c1ccno1 ; Cc1ncsc1-c1ccc([C@H](C)NC(=O)[C@@H]2C[C@@H](O)CN2C(=O)[C@@H](c2ccno2)C(C)C)cc1N]

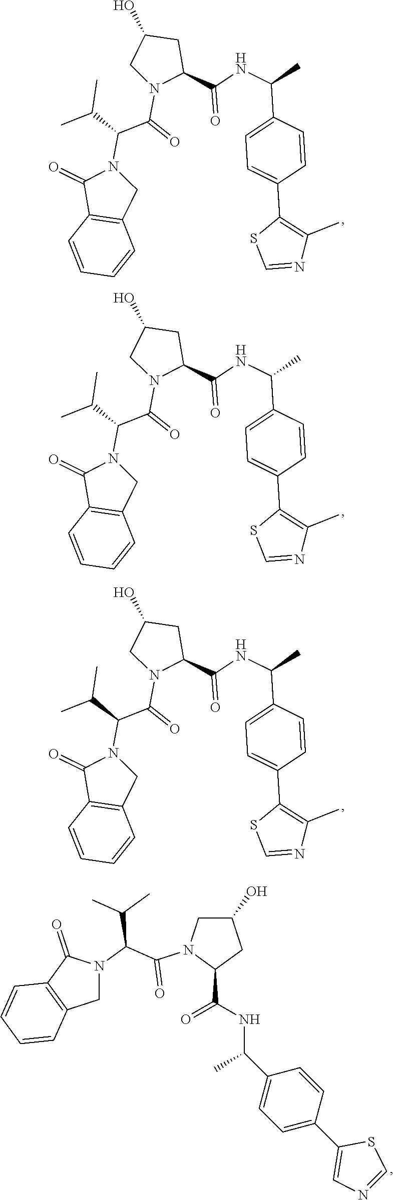

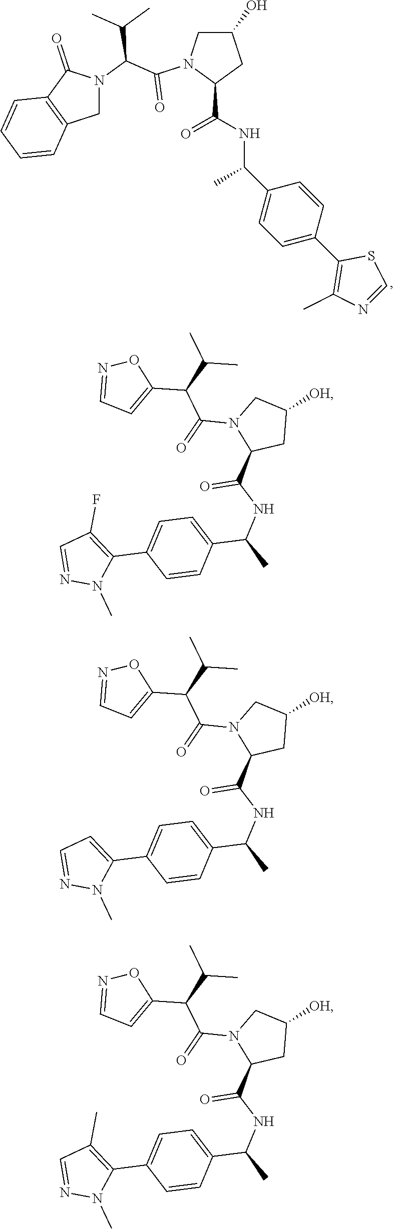 [SMILES: Cc1ncsc1-c1ccc([C@H](C)NC(=O)[C@@H]2C[C@@H](O)CN2C(=O)[C@H](C(C)C)N2Cc3ccccc3C2=O)cc1 ; CC(C)[C@@H](C(=O)N1C[C@H](O)C[C@H]1C(=O)N[C@@H](C)c1ccc(-c2c(F)cnn2C)cc1)c1ccno1 ; CC(C)[C@@H](C(=O)N1C[C@H](O)C[C@H]1C(=O)N[C@@H](C)c1ccc(-c2ccnn2C)cc1)c1ccno1 ; Cc1cnn(C)c1-c1ccc([C@H](C)NC(=O)[C@@H]2C[C@@H](O)CN2C(=O)[C@@H](c2ccno2)C(C)C)cc1]

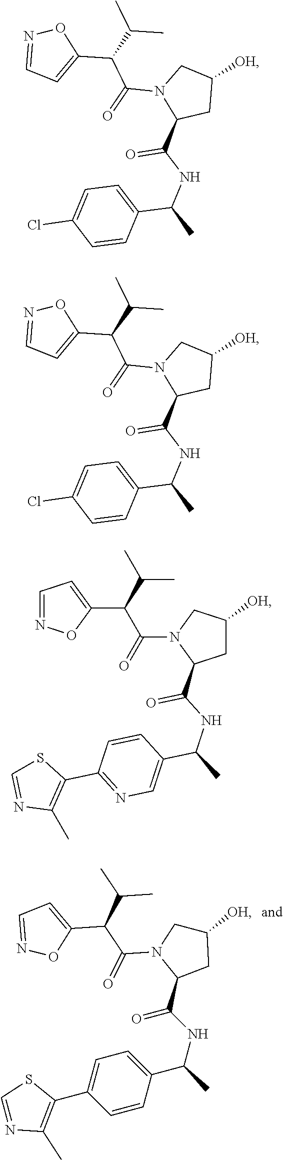

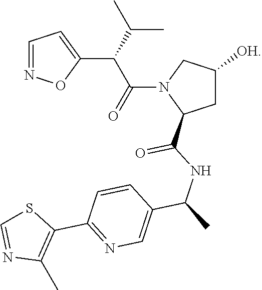 [SMILES: Cc1ncsc1-c1ccc([C@H](C)NC(=O)[C@@H]2C[C@@H](O)CN2C(=O)[C@H](c2ccno2)C(C)C)cn1]

In any aspect or embodiment described herein, one of R₁₄ₐ and R_{14b} is a H, C₁₋₃ alkyl (e.g., methyl), Cl fluoroalkyl, CHF₂, or CF₃, and the other is a H.

In any aspect or embodiment described herein, one of R₁₄ₐ and R_{14b} is a H, C₁₋₃ alkyl (e.g., methyl), and the other is a H.

In any aspect or embodiment described herein, the ULM is selected from:

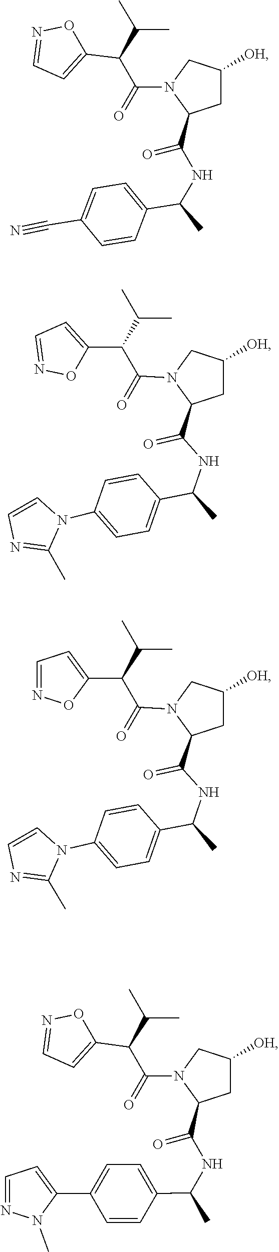 [SMILES: CC(C)[C@@H](C(=O)N1C[C@H](O)C[C@H]1C(=O)N[C@@H](C)c1ccc(C#N)cc1)c1ccno1 ; Cc1nccn1-c1ccc([C@H](C)NC(=O)[C@@H]2C[C@@H](O)CN2C(=O)[C@H](c2ccno2)C(C)C)cc1 ; Cc1nccn1-c1ccc([C@H](C)NC(=O)[C@@H]2C[C@@H](O)CN2C(=O)[C@@H](c2ccno2)C(C)C)cc1 ; CC(C)[C@@H](C(=O)N1C[C@H](O)C[C@H]1C(=O)N[C@@H](C)c1ccc(-c2ccnn2C)cc1)c1ccno1]

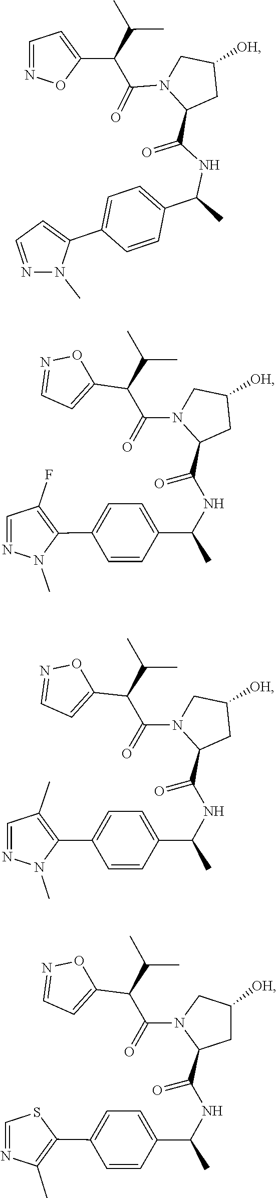 [SMILES: CC(C)[C@@H](C(=O)N1C[C@H](O)C[C@H]1C(=O)N[C@@H](C)c1ccc(-c2ccnn2C)cc1)c1ccno1 ; CC(C)[C@@H](C(=O)N1C[C@H](O)C[C@H]1C(=O)N[C@@H](C)c1ccc(-c2c(F)cnn2C)cc1)c1ccno1 ; Cc1cnn(C)c1-c1ccc([C@H](C)NC(=O)[C@@H]2C[C@@H](O)CN2C(=O)[C@@H](c2ccno2)C(C)C)cc1 ; Cc1ncsc1-c1ccc([C@H](C)NC(=O)[C@@H]2C[C@@H](O)CN2C(=O)[C@@H](c2ccno2)C(C)C)cc1]

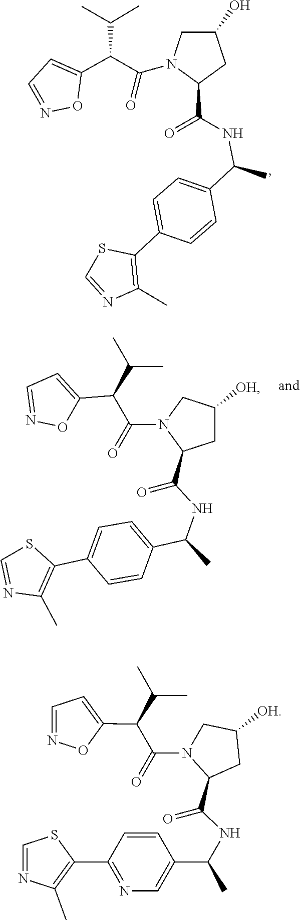

In any aspect or embodiment described herein, the ULM is selected from:

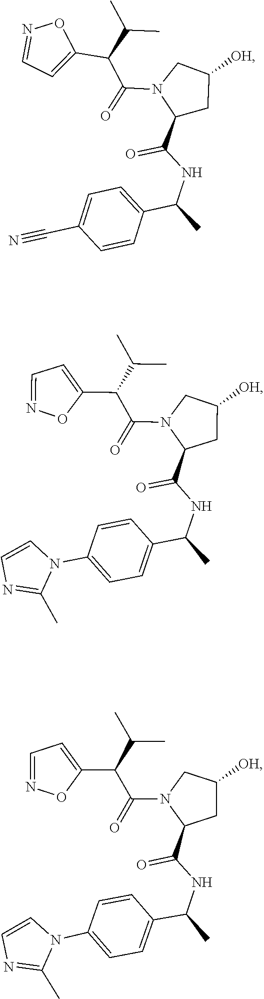 [SMILES: CC(C)[C@@H](C(=O)N1C[C@H](O)C[C@H]1C(=O)N[C@@H](C)c1ccc(C#N)cc1)c1ccno1 ; Cc1nccn1-c1ccc([C@H](C)NC(=O)[C@@H]2C[C@@H](O)CN2C(=O)[C@H](c2ccno2)C(C)C)cc1 ; Cc1nccn1-c1ccc([C@H](C)NC(=O)[C@@H]2C[C@@H](O)CN2C(=O)[C@@H](c2ccno2)C(C)C)cc1]

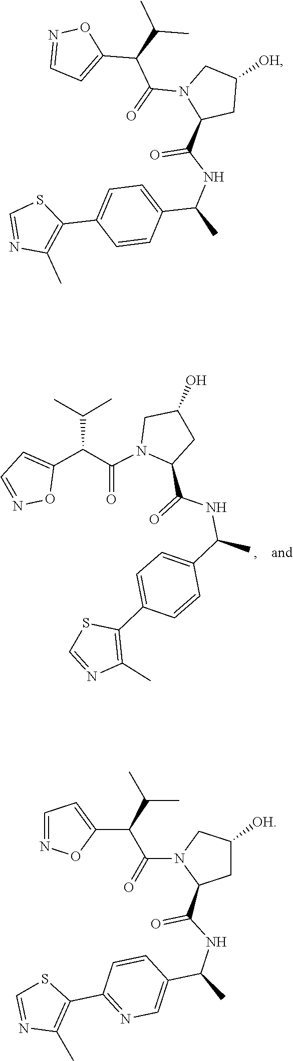 [SMILES: Cc1ncsc1-c1ccc([C@H](C)NC(=O)[C@@H]2C[C@@H](O)CN2C(=O)[C@@H](c2ccno2)C(C)C)cc1 ; Cc1ncsc1-c1ccc([C@H](C)NC(=O)[C@@H]2C[C@@H](O)CN2C(=O)[C@H](c2ccno2)C(C)C)cc1 ; Cc1ncsc1-c1ccc([C@H](C)NC(=O)[C@@H]2C[C@@H](O)CN2C(=O)[C@@H](c2ccno2)C(C)C)cn1]

In certain embodiments the compounds as described herein include a means for binding an E3 ubiquitin ligase, e.g., Von Hippel-Lindau E3 ubiquitin ligase. In certain embodiments the ULM is VLM and comprises a chemical structure selected from the group ULM-a:

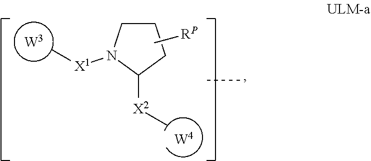

wherein:
a dashed line indicates the attachment of at least one PTM, another ULM or VLM (i.e., VLM′), or a chemical linker moiety coupling at least one PTM, a ULM′ or a VLM′ to the other end of the linker;
X¹, X² of Formula ULM-a are each independently selected from the group of a bond, 0, NR^{Y3}, CR^{Y3}R^{Y4}, C═O, C═S, SO, and SO₂;
R^{Y3}, R^{Y4} of Formula ULM-a are each independently selected from the group of H, linear or branched C₁₋₆ alkyl, optionally substituted by 1 or more halo, optionally substituted C₁₋₆ alkoxyl (e.g., optionally substituted by 0-3 R^{P} groups);
R^{P} of Formula ULM-a is 0, 1, 2, or 3 groups, each independently selected from the group H, halo, —OH, C₁₋₃ alkyl, C═O, alkyl, alkoxy or a combination thereof;
W³ of Formula ULM-a is selected from the group of an optionally substituted T, an optionally substituted -T-N(R^{1a}R^{1b})X³, optionally substituted -T-N(R^{1a}R^{1b}) optionally substituted -T-Aryl, an optionally substituted -T-Heteroaryl, an optionally substituted T-biheteroaryl, an optionally substituted -T-Heterocyclyl, an optionally substituted -T-biheterocyclyl, an optionally substituted —NR¹-T-Aryl, an optionally substituted —NR¹-T-Heteroaryl or an optionally substituted —NR¹-T-Heterocyclyl;
X³ of Formula ULM-a is C═O, R¹, R^{1a}, R^{1b};
each of R¹, R^{1a}, R^{1b} is independently selected from the group consisting of H, linear or branched C₁-C₆ alkyl group optionally substituted by 1 or more halo or —OH groups, R^{Y3}C=O, R^{Y3}C=S, R^{Y3}SO, R^{Y3}SO₂, N(R^{Y3}R^{Y4})C═O, N(R^{Y3}R^{Y4})C=S, N(R^{Y3}R^{Y4})SO, and N(R^{Y3}R^{Y4})SO₂;
T of Formula ULM-a is selected from the group of an optionally substituted alkyl, —(CH₂)ₙ— group, —(CH₂)ₙ—O—C₁-C₆ alkyl which is optionally substituted, linear, branched, or —(CH₂)ₙ— O-heterocyclyl which is optionally substituted, wherein each one of the methylene groups is optionally substituted with one or two substituents selected from the group of halogen, methyl, a linear or branched C₁-C₆ alkyl group optionally substituted by 1 or more halogen or —OH groups, an amino acid side chain optionally substituted or an optionally substituted heterocyclyl;
W⁴ of Formula ULM-a is an optionally substituted —NR1-T-Aryl wherein the aryl group may be optionally substituted with an optionally substituted 5-6 membered heteroaryl or an optionally substituted aryl, an optionally substituted —NR1-T-Heteroaryl group, wherein the heteroaryl is optionally substuted with an optionally substituted aryl or an optionally substituted heteroaryl, or an optionally substituted —NR1-T-Heterocyclyl, where —NR1 is covalently bonded to X² and R¹ is H or CH₃, preferably H.

In certain embodiments, R^{P} is modified to form a prodrug, including by an ester or ether linkage.

In any aspect or embodiment described herein, T is selected from the group of an optionally substituted alkyl, —(CH₂)ₙ— group, wherein each one of the methylene groups is optionally substituted with one or two substituents selected from the group of halogen, methyl, optionally substituted alkoxy, a linear or branched C₁-C₆ alkyl group optionally substituted by 1 or more halogen, C(O)NR¹R^{1a}, or NR¹R^{1a} or R¹ and R^{1a} are joined to form an optionally substituted heterocyclyl, or —OH groups or an amino acid side chain optionally substituted; and n is 0 to 6, often 0, 1, 2, or 3, preferably 0 or 1.

In any aspect or embodiment described herein, W⁴ of Formula ULM-a is

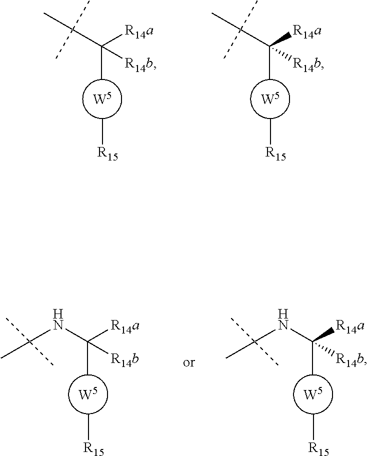

wherein R₁₄ₐ, R_{14b}, are each independently selected from the group of H, haloalkyl (e.g., fluoroalkyl), optionally substituted alkyl, optionally substituted alkoxy, optionally substituted hydroxyl alkyl, optionally substituted alkylamine, optionally substituted amide, optionally substituted alkyl-amide, optionally substituted alkyl-cyano, optionally substituted alkyl-phosphate, optionally substituted heteroalkyl, optionally substituted alkyl-heterocycloalkyl, optionally substituted alkoxy-heterocycloalkyl, COR₂₆, alkyl-COR₂₆, CONR₂₇ₐR_{27b}, NHCOR₂₆, or NHCH₃COR₂₆; and the other of R₁₄ₐ and R_{14b} is H; or R₁₄ₐ, R_{14b}, together with the carbon atom to which they are attached, form an optionally substituted 3 to 5 membered cycloalkyl, heterocycloalkyl, spirocycloalkyl or spiroheterocyclyl, wherein the spiroheterocyclyl is not epoxide or aziridine.

In any of the embodiments, W⁵ of Formula ULM-a is selected from the group of an optionally substituted phenyl, an optionally substituted napthyl or an optionally substituted 5-10 membered heteroaryl,
R₁₅ of Formula ULM-a is selected from the group of H, halogen, CN, C≡CH, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐ SO₂R_{14b}, optionally substituted alkyl, optionally substituted haloalkyl, optionally substituted haloalkoxy, optionally substituted aryl, optionally substituted heteroaryl, optionally substituted cycloalkyl, or optionally substituted heterocyclyl;

In additional embodiments, W⁴ substituents for use in the present disclosure also include specifically (and without limitation to the specific compound disclosed) the W⁴ substituents which are found in the identified compounds disclosed herein. Each of these W⁴ substituents may be used in conjunction with any number of W³ substituents which are also disclosed herein.

In certain additional embodiments, ULM-a, is optionally substituted by 0-3 R^{P} groups in the pyrrolidine moiety. Each R^{P} is independently H, halo, —OH, C1-3alkyl, C═O.

In any of the embodiments described herein, the W³, W⁴ of Formula ULM-a can independently be covalently coupled to a linker which is attached one or more PTM groups, and wherein the dashed line indicates the site of attachment of at least one PTM, another ULM (ULM′) or a chemical linker moiety coupling at least one PTM or a ULM′ or both to ULM.

In certain embodiments, ULM is VHL and is represented by the structure:

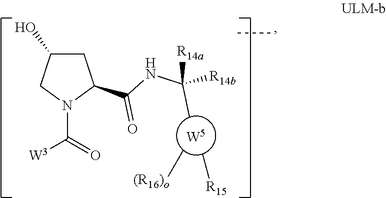

wherein:
W³ of Formula ULM-b is selected from the group of an optionally substituted aryl, optionally substituted heteroaryl, or

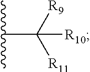

R₉ and R₁₀ of Formula ULM-b are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl;
R₁ᵢ of Formula ULM-b is selected from the group of an optionally substituted heterocyclyl, optionally substituted alkoxy, optionally substituted heteroaryl, optionally substituted aryl,

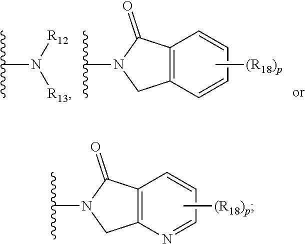

R₁₂ of Formula ULM-b is selected from the group of H or optionally substituted alkyl;
R₁₃ of Formula ULM-b is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₄ₐ, R_{14b} of Formula ULM-b, are each independently selected from the group of H, haloalkyl (e.g. fluoroalkyl), optionally substituted alkyl, optionally substitute alkoxy, aminomethyl, alkylaminomethyl, alkoxymethyl, optionally substituted hydroxyl alkyl, optionally substituted alkylamine, optionally substituted amide, optionally substituted alkyl-amide, optionally substituted alkyl-cyano, optionally substituted alkyl-phosphate, optionally substituted heteroalkyl, optionally substituted alkyl-heterocycloalkyl, optionally substituted alkoxy-heterocycloalkyl, COR₂₆, alkyl-COR₂₆, CONR₂₇ₐR_{27b}, CH₂NHCOR₂₆, or (CH₂)N(CH₃)COR₂₆; and the other of R₁₄ₐ and R_{14b} is H; or R₁₄ₐ, R_{14b}, together with the carbon atom to which they are attached, form an optionally substituted 3 to 6 membered cycloalkyl, heterocycloalky, spirocycloalkyl or spiroheterocyclyl, wherein the spiroheterocyclyl is not epoxide or aziridine;
W⁵ of Formula ULM-b is selected from the group of a phenyl, napthyl, or a 5-10 membered heteroaryl,
R₁₅ of Formula ULM-b is selected from the group of H, halogen, CN, C≡CH, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐ SO₂R_{27b}, optionally substituted alkyl, optionally substituted haloalkyl, optionally substituted haloalkoxy, optionally substituted aryl, optionally substituted heteroaryl, optionally substituted cycloalkyl, or optionally substituted heterocyclyl;
each R₁₆ of Formula ULM-b is independently selected from the group of halo, CN, optionally substituted alkyl, optionally substituted alkylamine, optionally substituted haloalkyl, hydroxy, or optionally substituted haloalkoxy;
o of Formula ULM-b is 0, 1, 2, 3, or 4;
R₁₈ of Formula ULM-b is independently selected from the group of H, halo, optionally substituted alkoxy, cyano, optionally substituted alkyl, haloalkyl, haloalkoxy or a linker;
each R₂₆ is independently selected from H, OH, optionally substituted alkyl or NR₂₇ₐR_{27b};
each R₂₇ₐ and R_{27b} is independently H, optionally substituted alkyl, optionally substituted 3-5 member cycloalkyl, or R₂₇ₐ and R_{27b} together with the nitrogen atom to which they are attached form a 4-6 membered heterocyclyl;
and
p of Formula ULM-b is 0, 1, 2, 3, or 4, and wherein the dashed line indicates the site of attachment of at least one PTM, another ULM (ULM′) or a chemical linker moiety coupling at least one PTM or a ULM′ or both to ULM.

In certain embodiments, R₁₅ of Formula ULM-b is

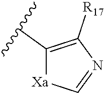

wherein R₁₇ is H, halo, optionally substituted C₃₋₆cycloalkyl, optionally substituted C₁₋₆alkyl, optionally substituted C₁₋₆alkenyl, and C₁₋₆haloalkyl; and Xa is S or O.

In certain embodiments, R₁₇ of Formula ULM-b is selected from the group methyl, ethyl, isopropyl, and cyclopropyl.

In certain additional embodiments, R₁₅ of Formula ULM-b is selected from the group consisting of:

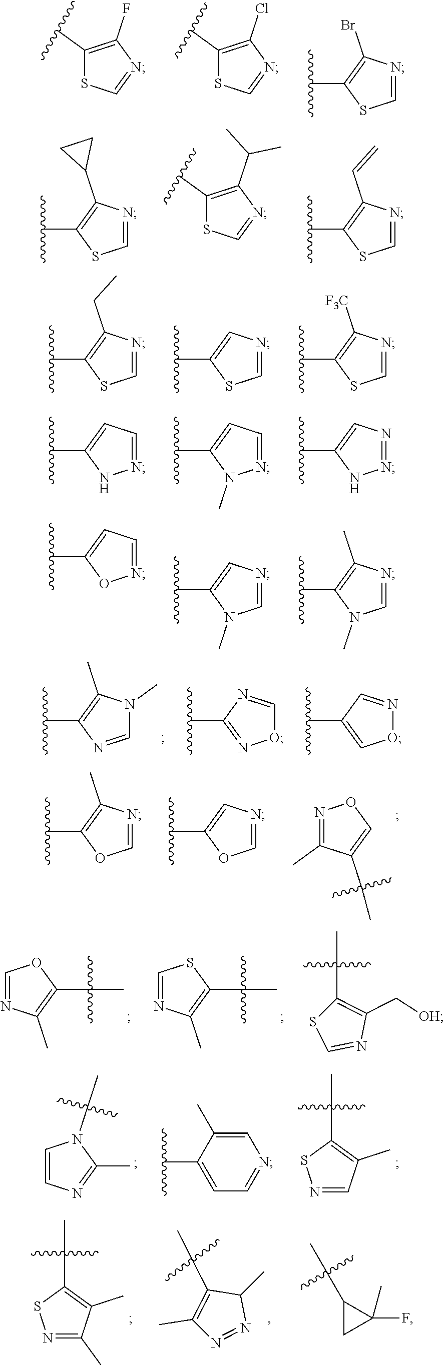

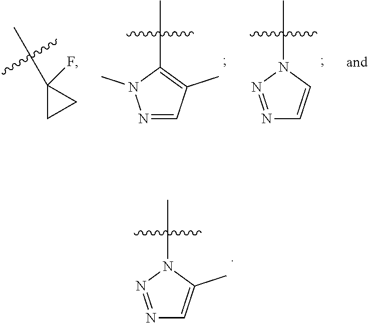

In certain embodiments, R₁₁ of Formula ULM-b is selected from the group consisting of:

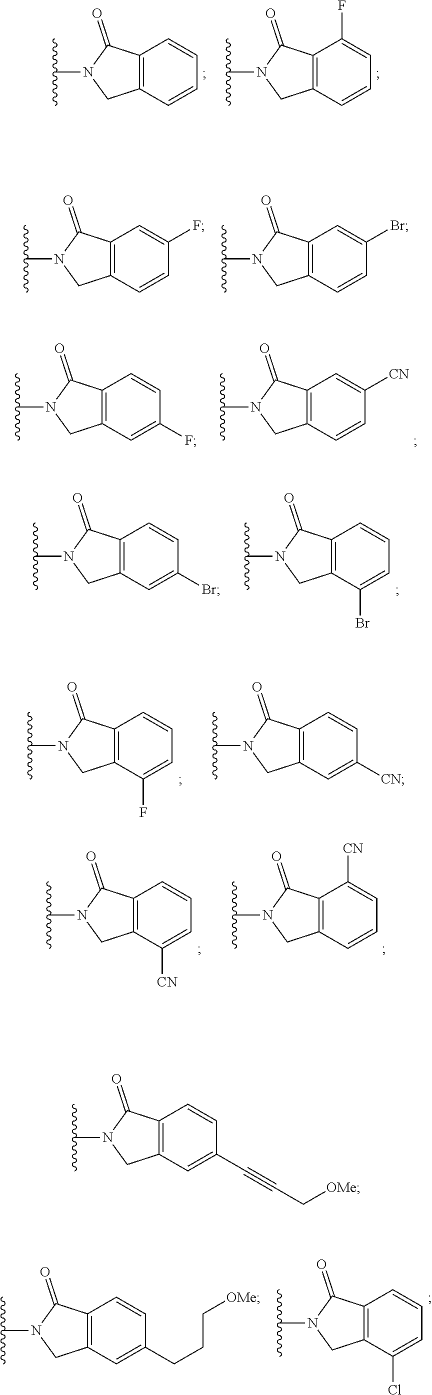

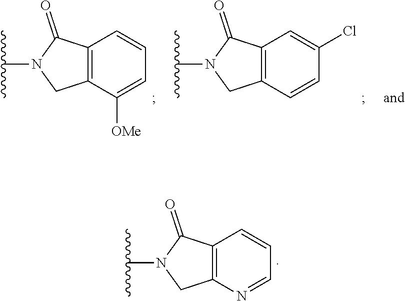

In any aspect or embodiment described herein, R₁₄ₐ, R₁₄ of Formula ULM-b, are each independently selected from the group of H, optionally substituted haloalkyl, optionally substituted alkyl, optionally substituted alkoxy, optionally substituted hydroxyl alkyl, optionally substituted alkylamine, optionally substituted amide, optionally substituted alkyl-amide, optionally substituted alkyl-cyano, optionally substituted alkyl-phosphate, optionally substituted heteroalkyl, optionally substituted alkyl-heterocycloalkyl, optionally substituted alkoxy-heterocycloalkyl, COR₂₆, alkyl-COR₂₆, CH₂OR₃₀, CH₂NHR₃₀, CH₂NCH₃R₃₀, CONR₂₇ₐR_{27b}, CH₂CONR₂₇ₐR_{27b}, CH₂NHCOR₂₆, or CH₂NCH₃COR₂₆; and the other of R₁₄ₐ and R_{14b} is H; or R₁₄ₐ, R_{14b}, together with the carbon atom to which they are attached, form an optionally substituted 3- to 6-membered cycloalkyl, heterocycloalkyl, spirocycloalkyl or spiroheterocyclyl, wherein the spiroheterocyclyl is not epoxide or aziridine, the said spirocycloalkyl or spiroheterocycloalkyl itself being optionally substituted with an alkyl, a haloalkyl, or —COR₃₃ where R₃₃ is an alkyl or a haloalkyl,
wherein R₃₀ is selected from H, alkyl, alkynylalkyl, cycloalkyl, heterocycloalkyl, cycloalkylalkyl, heterocycloalkylalkyl, arylalkyl or heteroarylalkyl further optionally substituted; R₂₆ and R₂₇ are as described above.

In any aspect or embodiment described herein, R₁₄ₐ, R_{14b} of Formula ULM-b, are each independently selected from the group of H, optionally substituted haloalkyl, optionally substituted alkyl, CH₂OR₃₀, CH₂NHR₃₀, CH₂NCH₃R₃₀, CONR₂₇ₐR_{27b}, CH₂CONR₂₇ₐR_{27b}, CH₂NHCOR₂₆, or CH₂NCH₃COR₂₆; and the other of R₁₄ₐ and R_{14b} is H; or R₁₄ₐ, R_{14b}, together with the carbon atom to which they are attached, form an optionally substituted 3- to 6-membered spirocycloalkyl or spiroheterocyclyl, wherein the spiroheterocyclyl is not epoxide or aziridine, the said spirocycloalkyl or spiroheterocycloalkyl itself being optionally substituted with an alkyl, a haloalkyl, or —COR₃₃ where R₃₃ is an alkyl or a haloalkyl, wherein R₃₀ is selected from H, alkyl, alkynylalkyl, cycloalkyl, heterocycloalkyl, cycloalkylalkyl, heterocycloalkylalkyl, arylalkyl or heteroarylalkyl further optionally substituted;

In certain embodiments, ULM has a chemical structure selected from the group of:

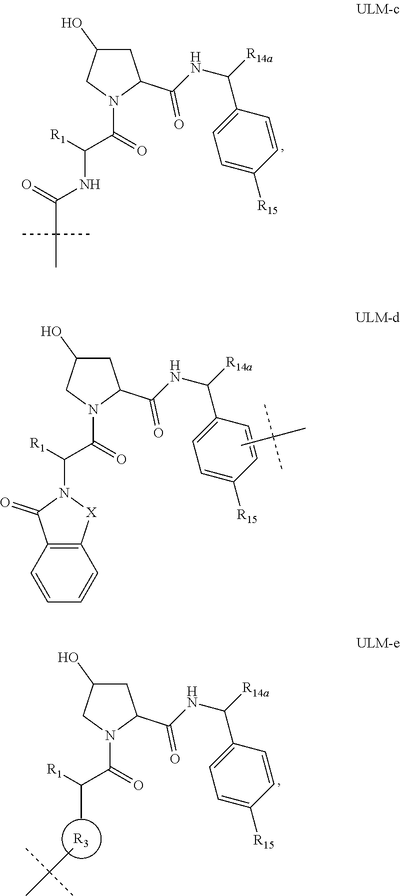

wherein:
R₁ of Formulas ULM-c, ULM-d, and ULM-e is H, ethyl, isopropyl, tert-butyl, sec-butyl, cyclopropyl, cyclobutyl, cyclopentyl, or cyclohexyl; optionally substituted alkyl, optionally substituted cycloalkyl, optionally substituted hydroxyalkyl, optionally substituted heteroaryl, or haloalkyl;
R₁₄ₐ of Formulas ULM-c, ULM-d, and ULM-e is H, haloalkyl, optionally substituted alkyl, methyl, fluoromethyl, hydroxymethyl, ethyl, isopropyl, or cyclopropyl;
R₁₅ of Formulas ULM-c, ULM-d, and ULM-e is selected from the group consisting of H, halogen, CN, C≡CH, OH, NO₂, optionally substituted heteroaryl, optionally substituted aryl; optionally substituted alkyl, optionally substituted haloalkyl, optionally substituted haloalkoxy, optionally substituted cycloalkyl, or optionally substituted heterocyclyl;
X of Formulas ULM-c, ULM-d, and ULM-e is C, CH₂, or C═O
R₃ of Formulas ULM-c, ULM-d, and ULM-e is absent or an optionally substituted 5 or 6 membered heteroaryl; and
the dashed line indicates the site of attachment of at least one PTM, another ULM (ULM′) or a chemical linker moiety coupling at least one PTM or a ULM′ or both to ULM.

In certain embodiments, ULM comprises a group according to the chemical structure:

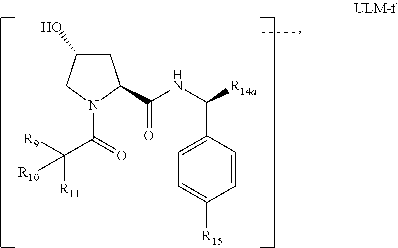

wherein:
R₁₄ₐ of Formula ULM-f is H, haloalkyl, optionally substituted alkyl, methyl, fluoromethyl, hydroxymethyl, ethyl, isopropyl, or cyclopropyl;
R₉ of Formula ULM-f is H;
R₁₀ of Formula ULM-f is H, ethyl, isopropyl, tert-butyl, sec-butyl, cyclopropyl, cyclobutyl, cyclopentyl, or cyclohexyl;
R₁₁ of Formula ULM-f is

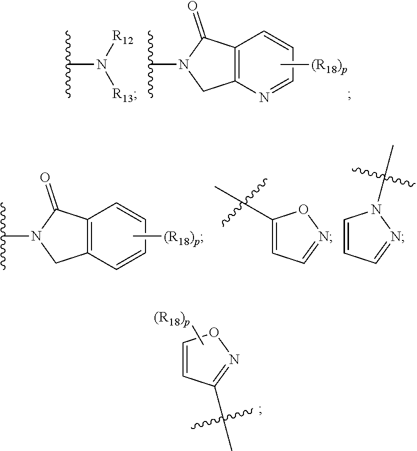

or optionally substituted heteroaryl;
p of Formula ULM-f is 0, 1, 2, 3, or 4;
each R₁₈ of Formula ULM-f is independently halo, optionally substituted alkoxy, cyano, optionally substituted alkyl, haloalkyl, haloalkoxy or a linker;
R₁₂ of Formula ULM-f is H, C═O;
R₁₃ of Formula ULM-f is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl,
R₁₅ of Formula ULM-f is selected from the group consisting of H, halogen, Cl, CN, C≡CH, OH, NO₂, optionally substituted haloalkyl, optionally substituted heteroaryl, optionally substituted aryl;

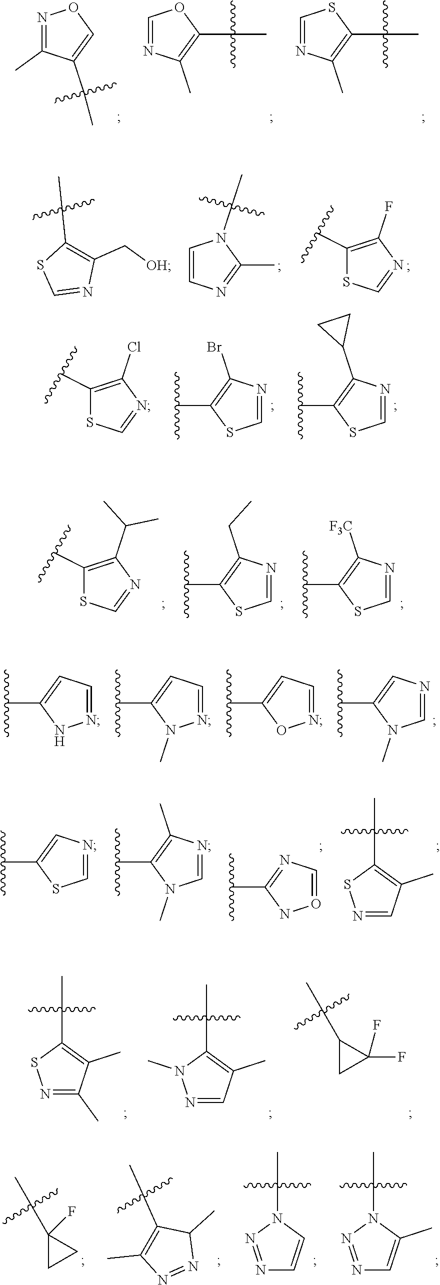

and
wherein the dashed line of Formula ULM-f indicates the site of attachment of at least one PTM, another ULM (ULM′) or a chemical linker moiety coupling at least one PTM or a ULM′ or both to ULM.

In certain embodiments, the ULM is selected from the following structures:

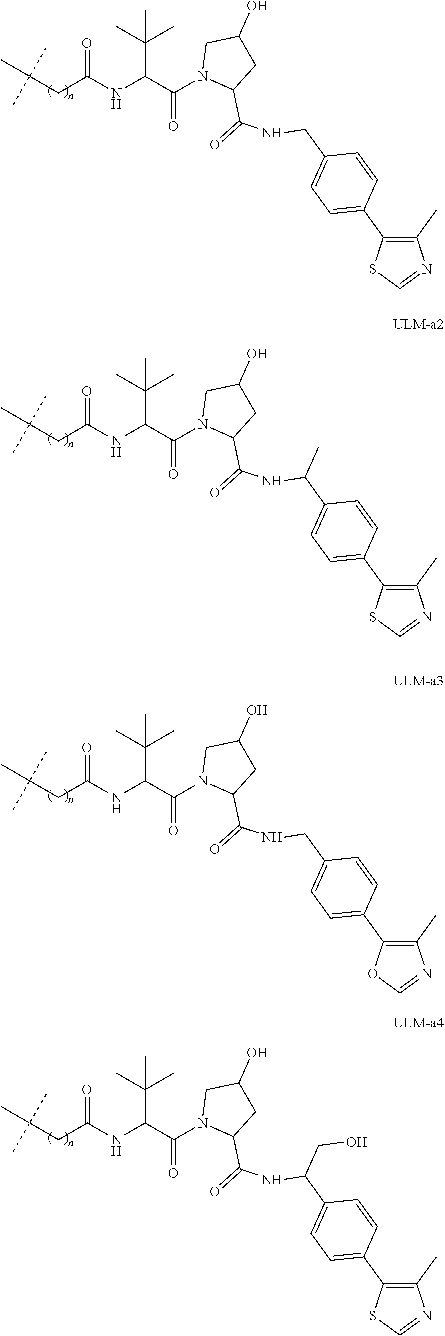

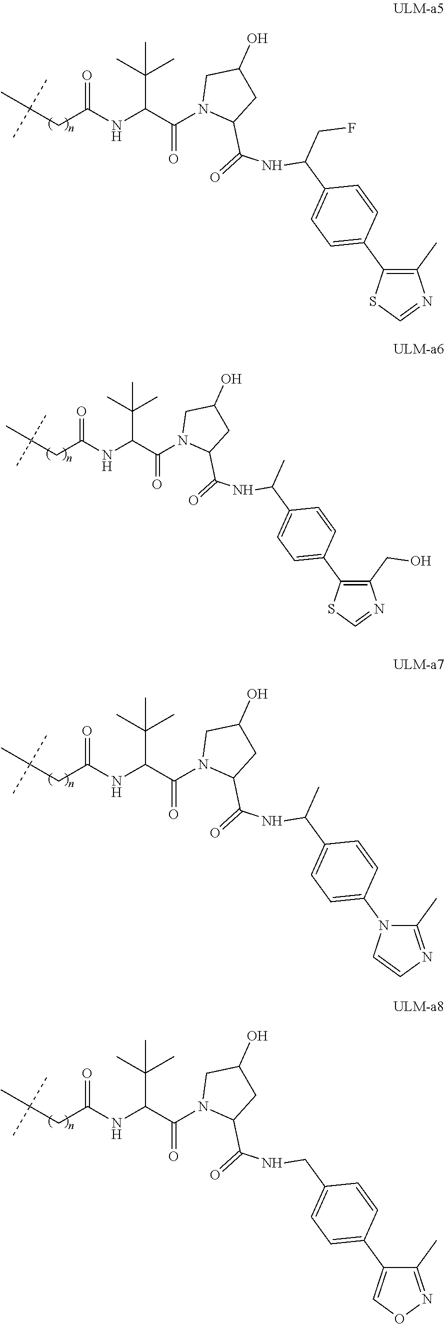

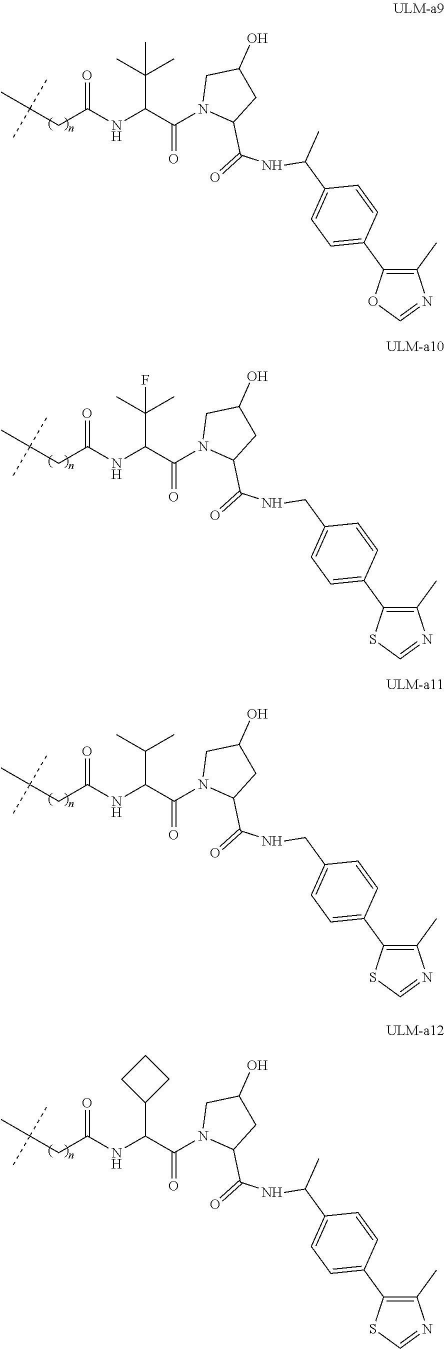

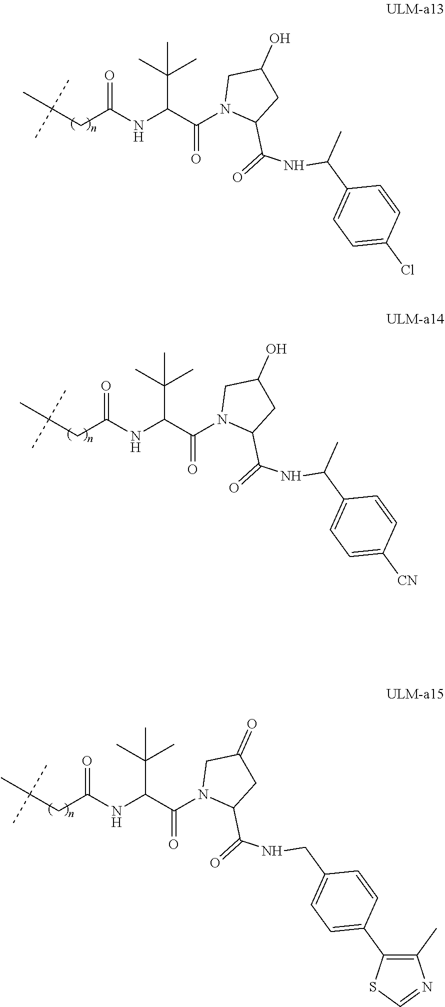

wherein n is 0 or 1.

In certain embodiments, the ULM is selected from the following structures:

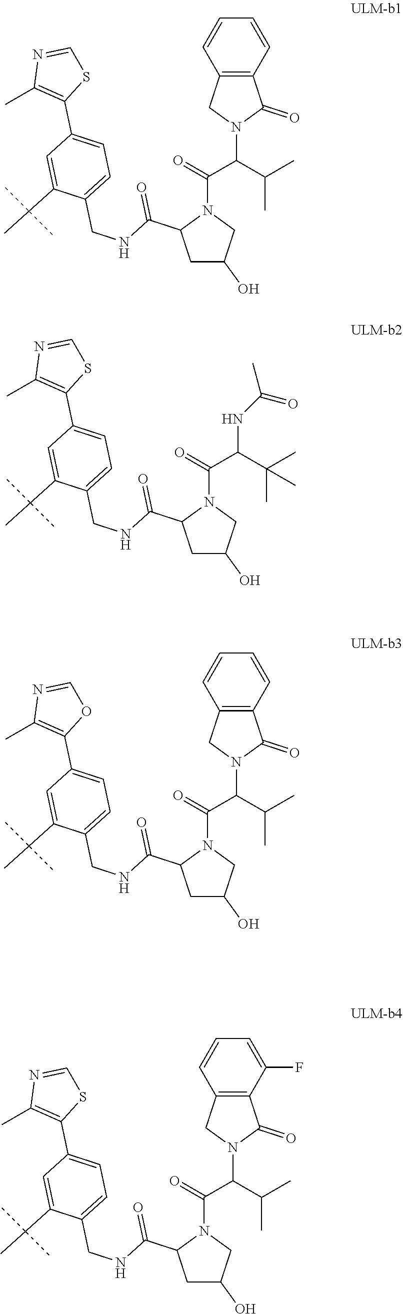

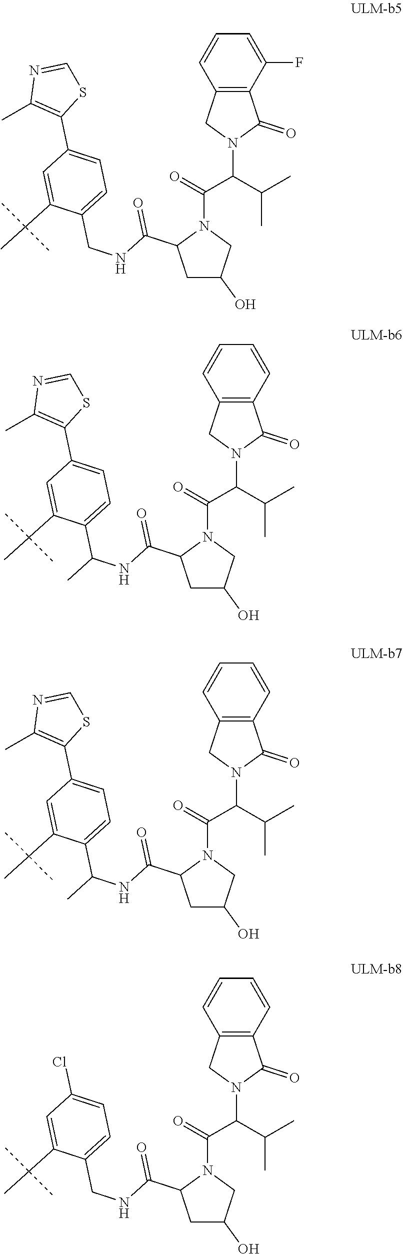

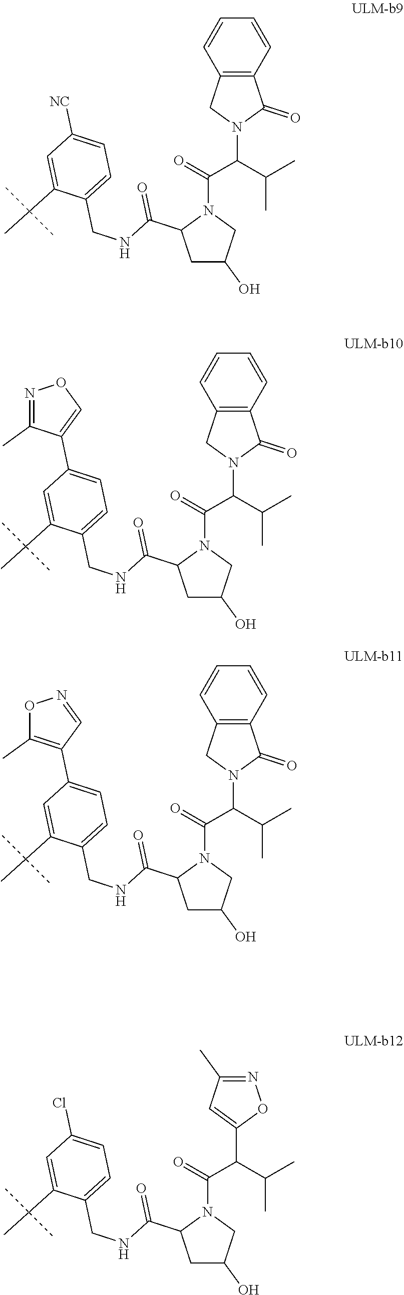

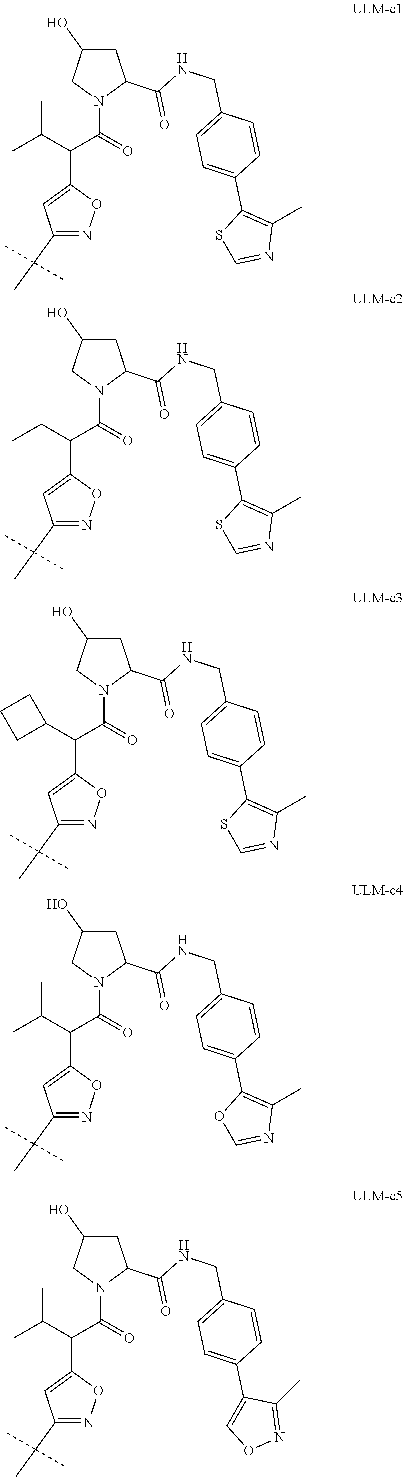

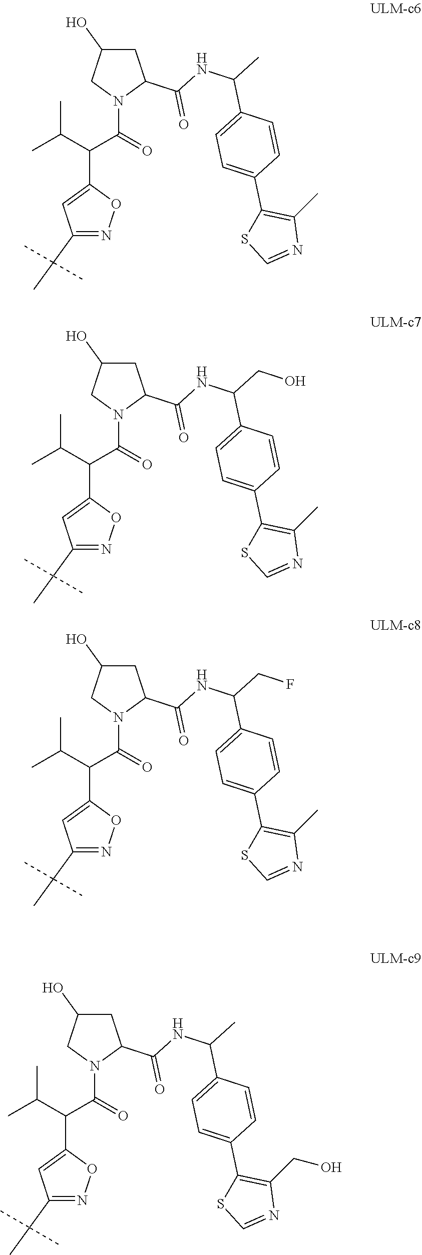

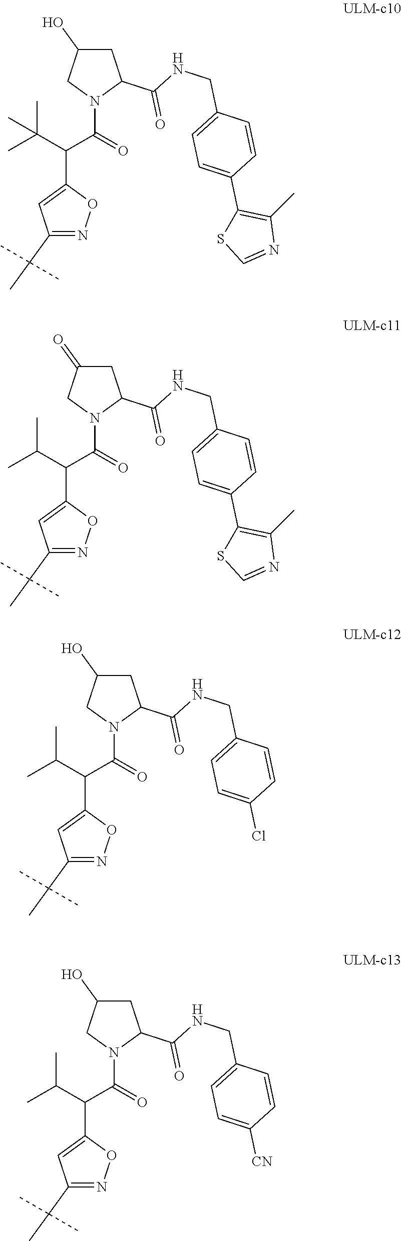

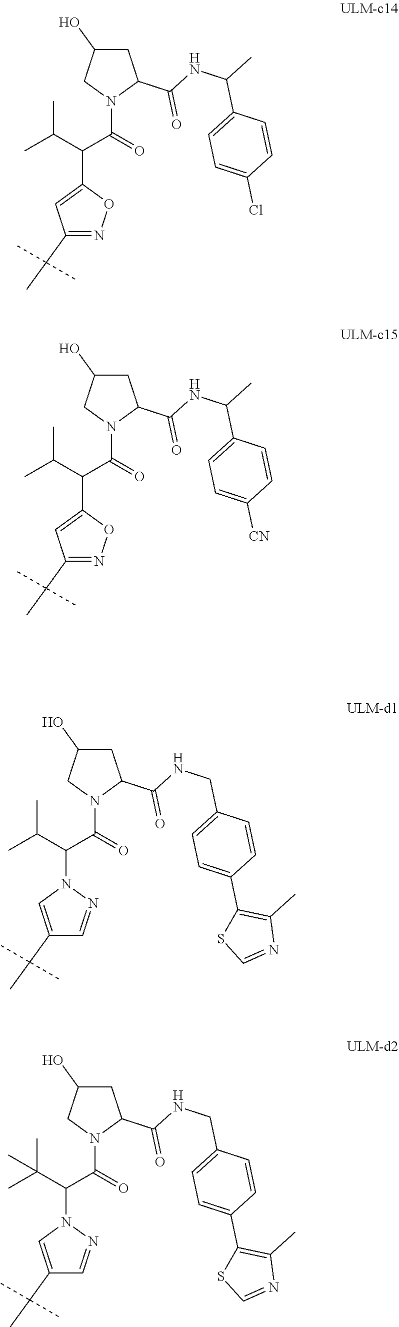

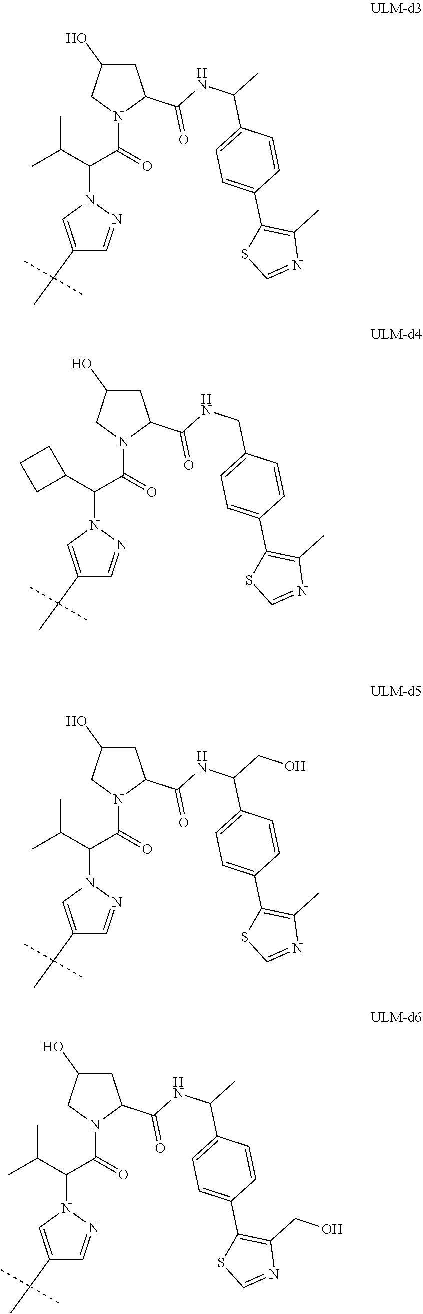

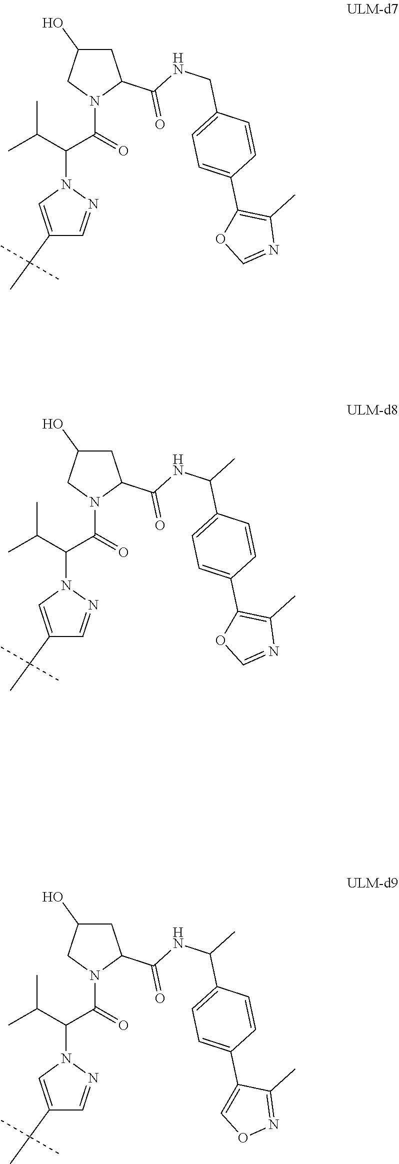

wherein, the phenyl ring in ULM-a1 through ULM-a15, ULM-b1 through ULM-b12, ULM-c1 through ULM-c15 and ULM-d1 through ULM-d9 is optionally substituted with fluorine, lower alkyl and alkoxy groups, and wherein the dashed line indicates the site of attachment of at least one PTM, another ULM (ULM′) or a chemical linker moiety coupling at least one PTM or a ULM′ or both to ULM-a.

In one embodiment, the phenyl ring in ULM-a1 through ULM-a15, ULM-b1 through ULM-b12, ULM-c1 through ULM-c15 and ULM-d1 through ULM-d9 can be functionalized as the ester to make it a part of the prodrug.

In certain embodiments, the hydroxyl group on the pyrrolidine ring of ULM-al through ULM-a15, ULM-b1 through ULM-b12, ULM-c1 through ULM-c15 and ULM-d1 through ULM-d9, respectively, comprises an ester-linked prodrug moiety.

In still additional embodiments, preferred compounds include those according to the chemical structure:

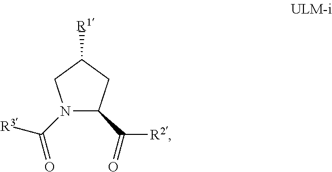

wherein:
R^{1′} of ULM-i is OH or a group which is metabolized in a patient or subject to OH;
R^{2′} of ULM-i is a —NH—CH₂-Aryl-HET (preferably, a phenyl linked directly to a methyl substituted thiazole);
R^{3′} of ULM-i is a —CHR^{CR3′}—NH—C(O)—R^{3P1} group or a —CHR^{CR3}—R^{3P2} group;
R^{CR3′} of ULM-i is a C₁-C₄ alkyl group, preferably methyl, isopropyl or tert-butyl;
R^{3P1} of ULM-i is C₁-C₃ alkyl (preferably methyl), an optionally substituted oxetane group (preferably methyl substituted, a —(CH₂)ₙOCH₃ group where n is 1 or 2 (preferably 2), or a

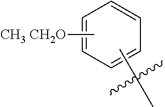

group (the ethyl ether group is preferably meta-substituted on the phenyl moiety), a morpholino group (linked to the carbonyl at the 2- or 3-position;
R^{3P2} of ULM-i is a

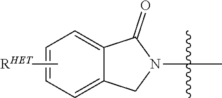

group;
Aryl of ULM-i is phenyl;
HET of ULM-i is an optionally substituted thiazole or isothiazole; and
R^{HET} of ULM-i is H or a halo group (preferably H);
or a pharmaceutically acceptable salt, stereoisomer, solvate or polymorph thereof, wherein each of said compounds is optionally connected to a PTM group (including a ULM′ group) via a linker group.

In certain aspects, bifunctional compounds comprising a ubiquitin E3 ligase binding moiety (ULM), wherein ULM is a group according to the chemical structure:

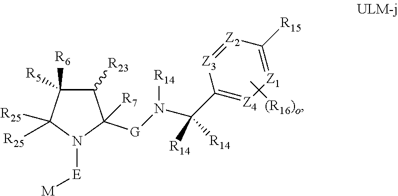

wherein:
each R₅ and R₆ of ULM-j is independently OH, SH, or optionally substituted alkyl or R₅, R₆, and the carbon atom to which they are attached form a carbonyl;
R₇ of ULM-j is H or optionally substituted alkyl;
E of ULM-j is a bond, C═O, or C=S;
G of ULM-j is a bond, optionally substituted alkyl, —COOH or C=J;
J of ULM-j is O or N—R₈;
R₈ of ULM-j is H, CN, optionally substituted alkyl or optionally substituted alkoxy;
M of ULM-j is optionally substituted aryl, optionally substituted heteroaryl, optionally substituted heterocyclyl or

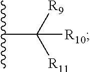

each R₉ and R₁₀ of ULM-j is independently H; optionally substituted alkyl, optionally substituted cycloalkyl, optionally substituted hydroxyalkyl, optionally substituted thioalkyl, a disulphide linked ULM, optionally substituted heteroaryl, or haloalkyl; or R₉, R₁₀, and the carbon atom to which they are attached form an optionally substituted cycloalkyl;
R₁₁ of ULM-j is optionally substituted heterocyclyl, optionally substituted alkoxy, optionally substituted heteroaryl, optionally substituted aryl, or

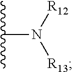

R₁₂ of ULM-j is H or optionally substituted alkyl;
R₁₃ of ULM-j is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate,
each R₁₄ of ULM-j is independently H, haloalkyl, optionally substituted cycloalkyl, optionally substituted alkyl, an azetidine, optionally substituted alkoxy, or optionally substituted heterocyclyl;
R₁₅ of ULM-j is H, CN, optionally substituted heteroaryl, haloalkyl, optionally substituted aryl, optionally substituted alkoxy, or optionally substituted heterocyclyl;
each R₁₆ of ULM-j is independently halo, optionally substituted alkyl, optionally substituted haloalkyl, optionally substituted CN, or optionally substituted haloalkoxy;
each R₂₅ of ULM-j is independently H or optionally substituted alkyl; or both R₂₅ groups can be taken together to form an oxo or optionally substituted cycloalkyl group;
R₂₃ of ULM-j is H or OH;
Z₁, Z₂, Z₃, and Z₄ of ULM-j are independently C or N; and
o of ULM-j is 0, 1, 2, 3, or 4, or a pharmaceutically acceptable salt, stereoisomer, solvate or polymorph thereof.

In certain embodiments, wherein G of ULM-j is C=J, J is O, R₇ is H, each R₁₄ is H, and o is 0.

In certain embodiments, wherein G of ULM-j is C=J, J is O, R₇ is H, each R₁₄ is H, R₁₅ is optionally substituted heteroaryl, and o is 0. In other instances, E is C═O and M is

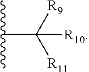

In certain embodiments, wherein E of ULM-j is C═O, R₁₁ is optionally substituted heterocyclyl or

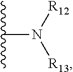

### and M is

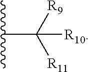

In certain embodiments, wherein E of ULM-j is C═O, M is

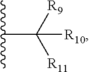

### and R₁₁ is

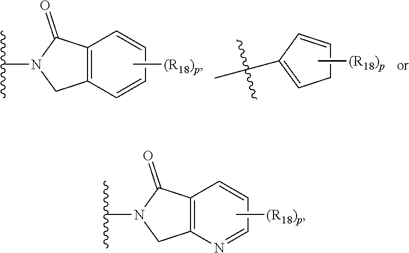

each R₁₈ is independently H, halo, optionally substituted alkoxy, cyano, optionally substituted alkyl, haloalkyl, or haloalkoxy; and p is 0, 1, 2, 3, or 4.

In certain embodiments, each R₁₄ is independently substituted with at least one of H, hydroxyl, halo, amine, amide, alkoxy, alkyl, haloalkyl, or heterocyclic.

In certain embodiments, R₁₅ of ULM-j is a group according to

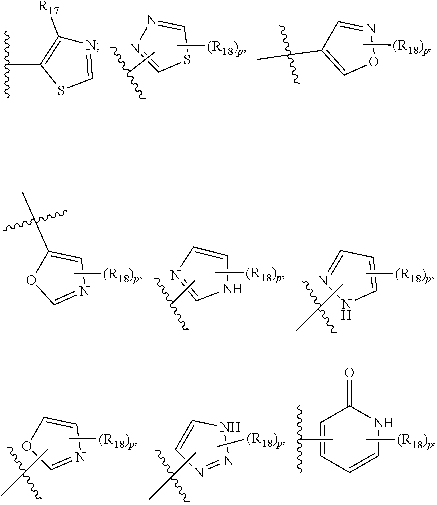

CN, or a haloalkyl, and each R₁₈ is independently H, halo, optionally substituted alkoxy, cyano, aminoalkyl, amidoalkyl, optionally substituted alkyl, haloalkyl, or haloalkoxy; and p is 0, 1, 2, 3, or 4.

In certain embodiments, ULM and where present, ULM′, are each independently a group according to the chemical structure:

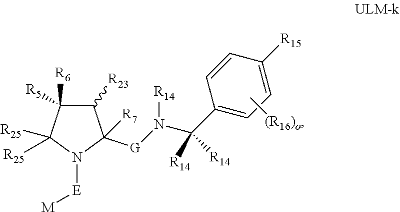

wherein:
G of ULM-k is C=J, J is O;
R₇ of ULM-k is H;
each R₁₄ of ULM-k is independently H, an amide, an alkyl, e.g., methyl, optionally substituted with one or more C₁-C₆ alkyl groups or C(O)NR′R″;
R′ and R″ are each independently H, optionally substituted alkyl, or cycloalkyl;
o of ULM-k is 0;
R₁₅ of ULM-k is defined as above for ULM-j;
R₁₆ of ULM-k is defined is as above for ULM-j; and
R₁₇ of ULM-k is H, halo, optionally substituted cycloalkyl, optionally substituted alkyl, optionally substituted alkenyl, and haloalkyl.

In other instances, R₁₇ of ULM-k is alkyl (e.g., methyl) or cycloalkyl (e.g., cyclopropyl).

In other embodiments, ULM and where present, ULM′, are each independently a group according to the chemical structure:

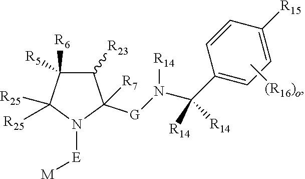

wherein:
G of ULM-k is C=J, J is O;
R₇ of ULM-k is H;
each R₁₄ of ULM-k is H;
o of ULM-k is 0; and
R₁₅ of ULM-k is selected from the group consisting of optionally substituted:

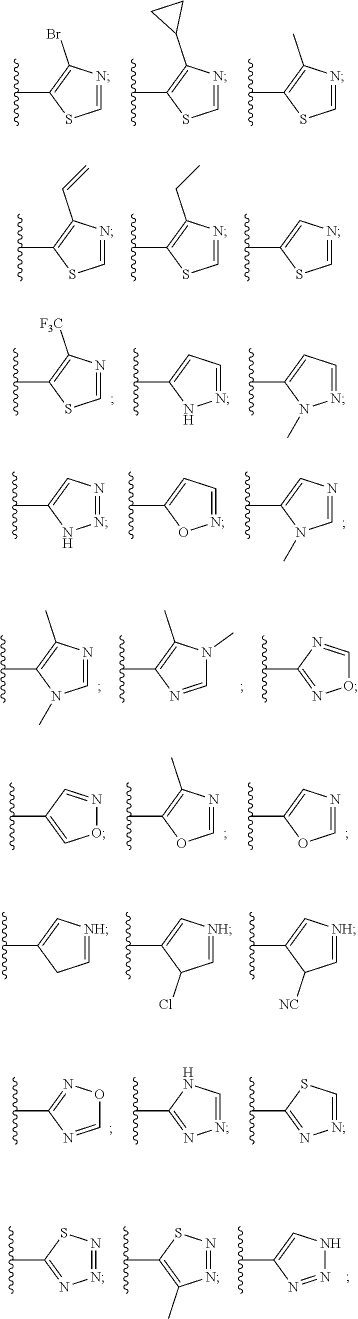

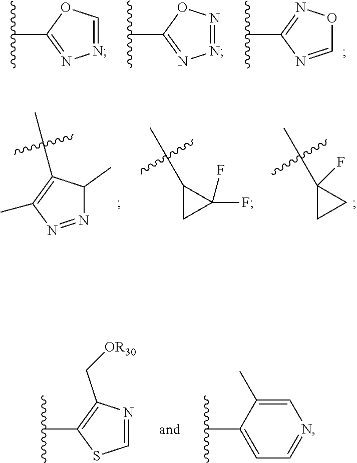

wherein R₃₀ of ULM-k is H or an optionally substituted alkyl.

In other embodiments, ULM and where present, ULM′, are each independently a group according to the chemical structure:

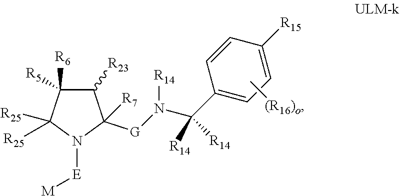

wherein:
E of ULM-k is C═O;
M of ULM-k is

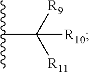

and
R₁₁ of ULM-k is selected from the group consisting of optionally substituted:

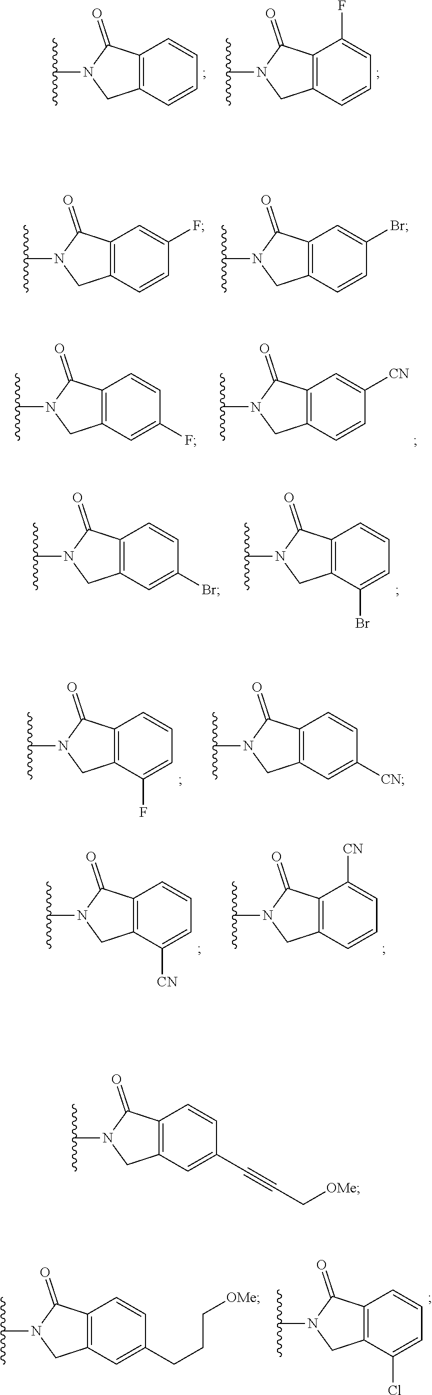

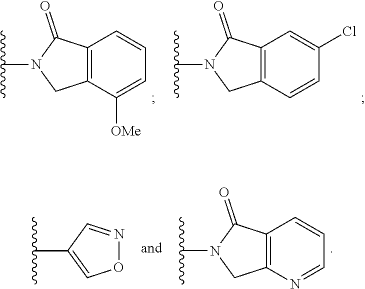

In still other embodiments, a compound of the chemical structure,

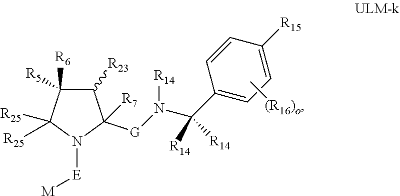

wherein:
E of ULM-k is C═O;
R₁₁ of ULM-k is

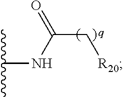

and
M of ULM-k is

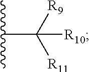

q of ULM-k is 1 or 2;
R₂₀ of ULM-k is H, optionally substituted alkyl, optionally substituted cycloalkyl, optionally substituted aryl, or

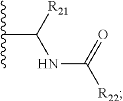

R₂₁ of ULM-k is H or optionally substituted alkyl; and
R₂₂ of ULM-k is H, optionally substituted alkyl, optionally substituted alkoxy, or haloalkyl.

In any embodiment described herein, R₁₁ of ULM-j or ULM-k is selected from the group consisting of:

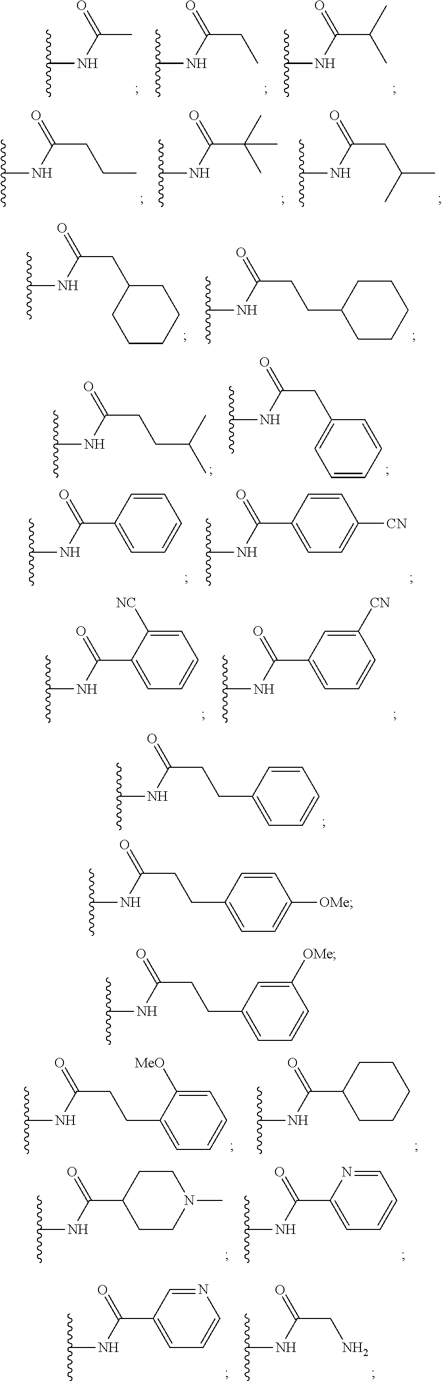

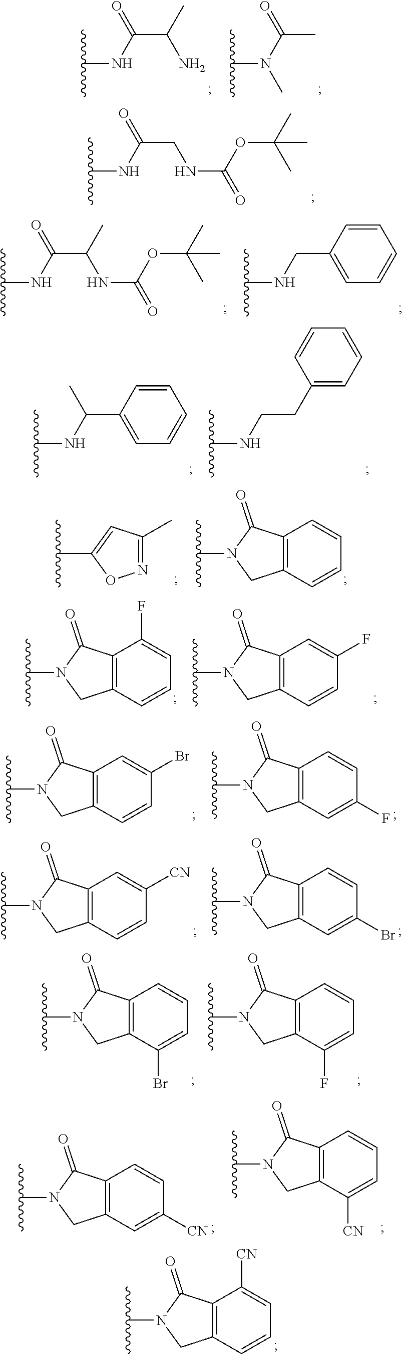

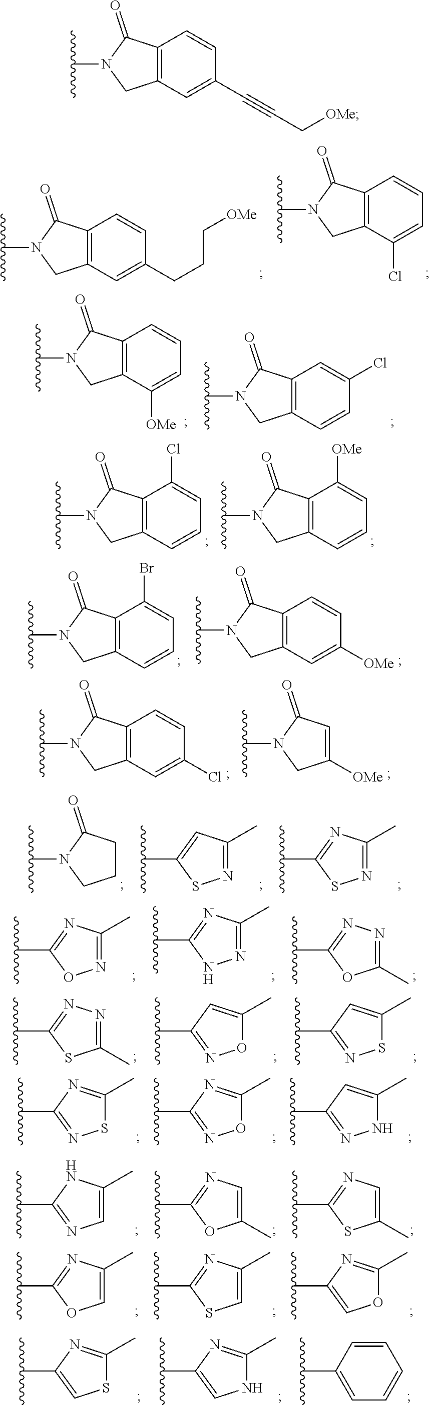

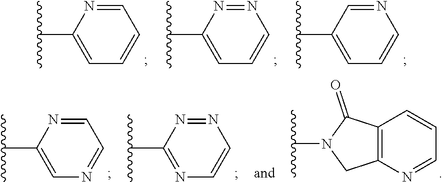

In certain embodiments, R₁₁ of ULM-j or ULM-k is selected from the group consisting of:

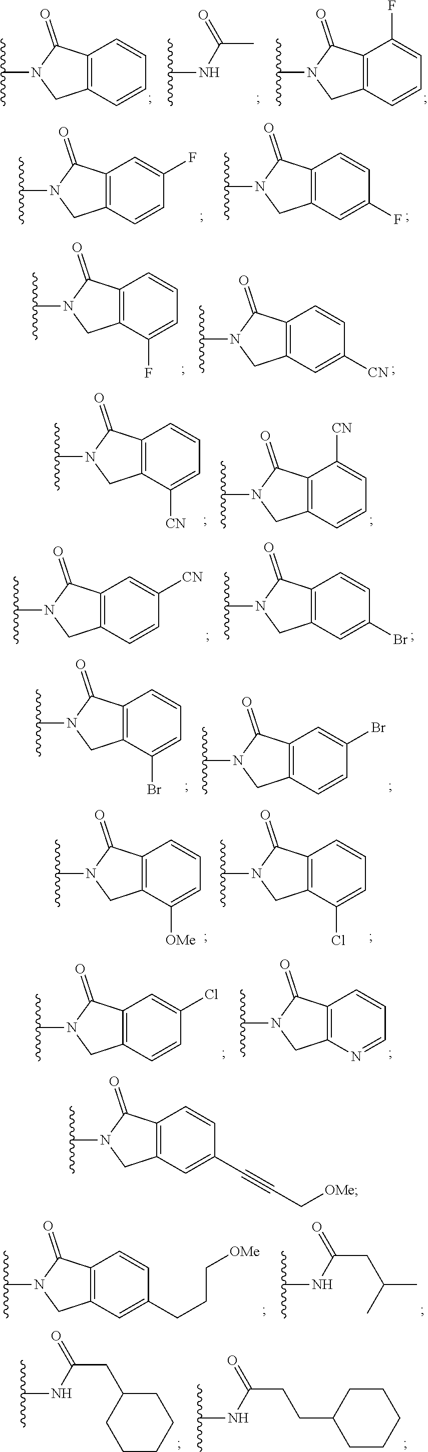

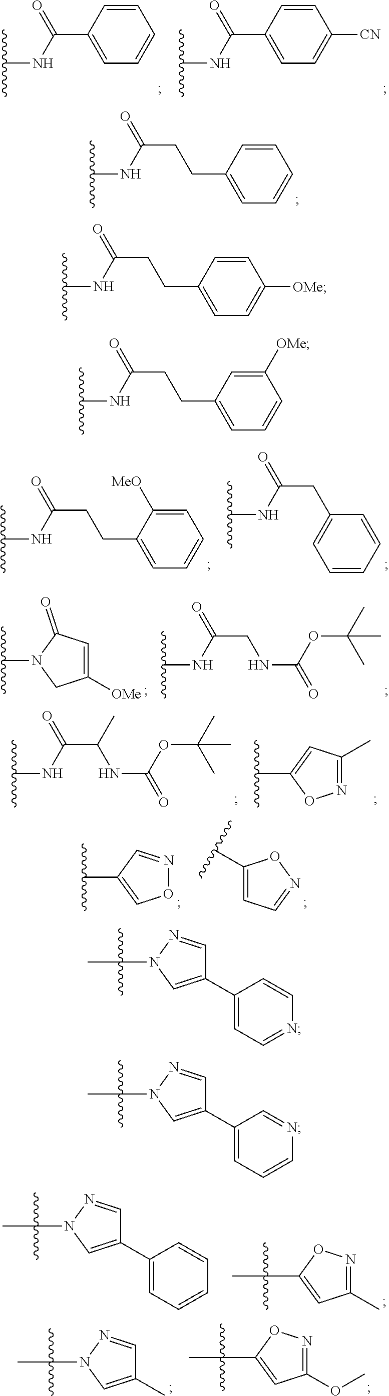

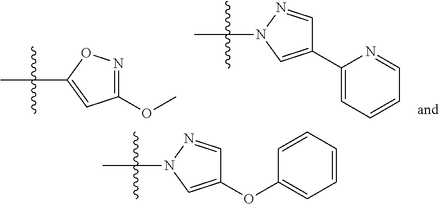

In certain embodiments, ULM (or when present ULM′) is a group according to the chemical structure:

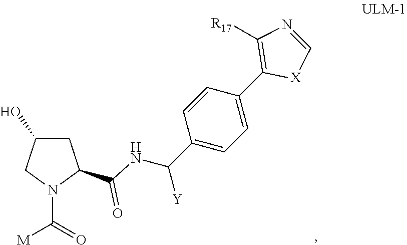

wherein:
X of ULM-1 is O or S;
Y of ULM-1 is H, methyl or ethyl;
R₁₇ of ULM-1 is H, methyl, ethyl, hydroxymethyl or cyclopropyl;
M of ULM-1 is optionally substituted aryl, optionally substituted heteroaryl, or

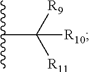

R₉ of ULM-1 is H;
R₁₀ of ULM-1 is H, optionally substituted alkyl, optionally substituted haloalkyl, optionally substituted heteroaryl, optionally substituted aryl, optionally substituted hydroxyalkyl, optionally substituted thioalkyl or cycloalkyl;
R11 of ULM-1 is optionally substituted heteroaromatic, optionally substituted heterocyclyl, optionally substituted aryl or

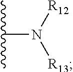

R₁₂ of ULM-1 is H or optionally substituted alkyl; and
R₁₃ of ULM-1 is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate.

In some embodiments, ULM and where present, ULM′, are each independently a group according to the chemical structure:

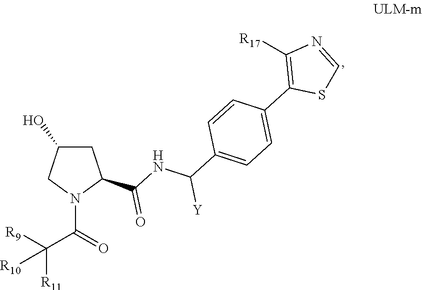

wherein:
Y of ULM-m is H, methyol or ethyl
R₉ of ULM-m is H;
R₁₀ is isopropyl, tert-butyl, sec-butyl, cyclopentyl, or cyclohexyl;
R₁₁ of ULM-m is optionally substituted amide, optionally substituted isoindolinone, optionally substituted isooxazole, optionally substituted heterocyclyls.

In other preferred embodiments of the disclosure, ULM and where present, ULM′, are each independently a group according to the chemical structure:

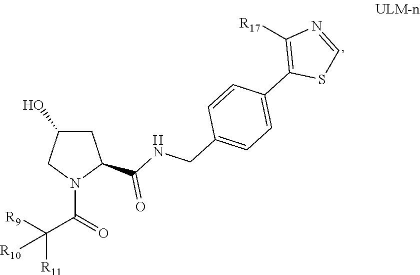

wherein:
R₁₇ of ULM-n is methyl, ethyl, or cyclopropyl; and
R₉, R₁₀, and R₁₁ of ULM-n are as defined above. In other instances, R₉ is H; and
R₁₀ of ULM-n is H, alkyl, or cycloalkyl (preferably, isopropyl, tert-butyl, sec-butyl, cyclopentyl, or cyclohexyl).

In other preferred embodiments of the disclosure, ULM and where present, ULM′, are each independently a group according to the chemical structure:

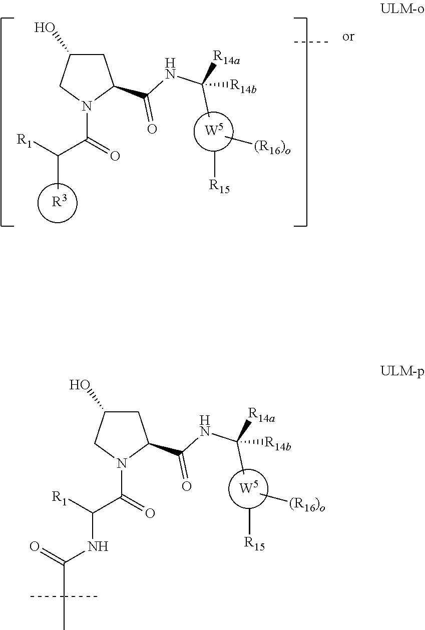

or a pharmaceutically acceptable salt thereof, wherein:
R₁ is H, optionally substituted alkyl or optionally substituted cycloalkyl;
R₃ is an optionally substituted 5-6 membered heteroaryl;
W⁵ is optionally substituted phenyl, optionally substituted napthyl or optionally substituted pyridinyl;
one of R₁₄ₐ and R_{14b} is H, optionally substituted alkyl, optionally substituted haloalkyl (e.g., fluoroalkyl), optionally substituted alkoxy, optionally substituted hydroxyl alkyl, optionally substituted alkylamine, optionally substituted heteroalkyl, optionally substituted alkyl-heterocycloalkyl, optionally substituted alkoxy-heterocycloalkyl, COR₂₆, CONR₂₇ₐR_{27b}, NHCOR₂₆, or NHCH₃COR₂₆; and the other of R₁₄ₐ and R_{14b} is H; or R₁₄ₐ, R_{14b}, together with the carbon atom to which they are attached, form an optionally substituted 3 to 6 membered cycloalkyl, heterocycloalkyl, spirocycloalkyl or spiroheterocyclyl, wherein the spiroheterocyclyl is not epoxide or aziridine;
R₁₅ is CN, optionally substituted fluoroalkyl,

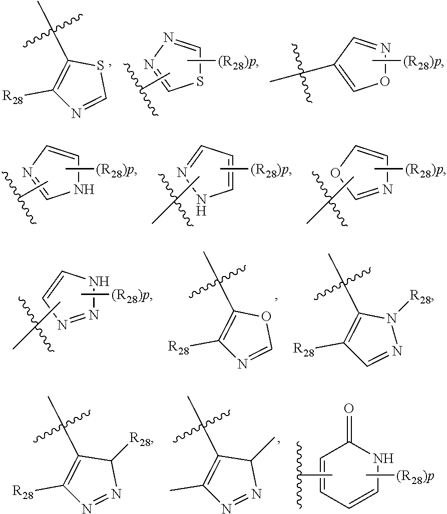

optionally substituted

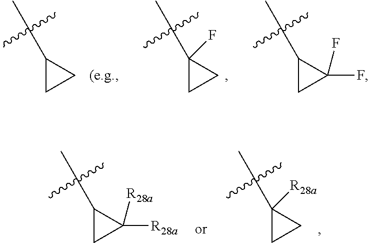

wherein R₂₈ₐ is halo, optionally substituted alkyl or fluoroalkyl, or

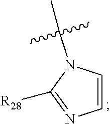

each R₁₆ is independently selected from halo, CN, optionally substituted alkyl, optionally substituted haloalkyl, hydroxy, or haloalkoxy;
each R₂₆ is independently H, optionally substituted alkyl or NR₂₇ₐR_{27b};
each R₂₇ₐ and R_{27b} is independently H, optionally substituted alkyl, optionally substituted cycloalkyl (e.g. optionally substituted 3-5 member cycloalkyl), or R₂₇ₐ and R_{27b} together with the nitrogen atom to which they are attached form a 4-6 membered heterocyclyl;
each R₂₈ is independently H, halogen, CN, optionally substituted aminoalkyl, optionally substituted amidoalkyl, optionally substituted haloalkyl, optionally substituted alkyl, optionally substituted alkoxy, optionally substituted heteroalkyl, optionally substituted alkylamine, optionally substituted hydroxyalkyl, amine, optionally substituted alkynyl, or optionally substituted cycloalkyl;
o is 0, 1 or 2; and
p is 0, 1, 2, 3, or 4.

In any of the aspects or embodiments described herein, the ULM is of the formula:

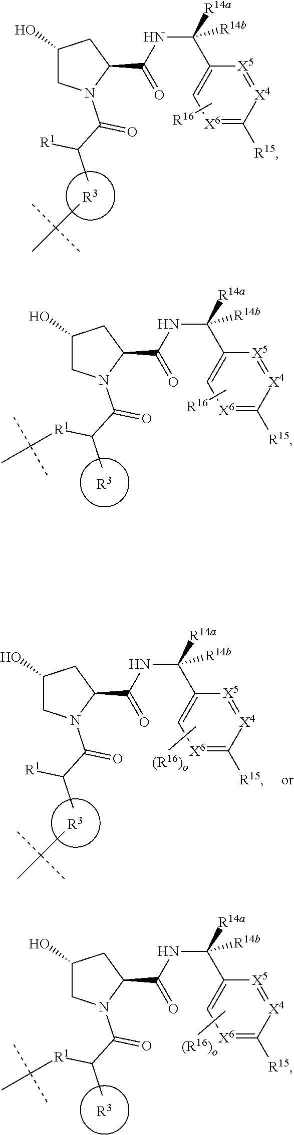

wherein:
each of X⁴, X⁵, and X⁶ is selected from CH and N, wherein no more than 2 are N;
R¹ is C₁₋₆ alkyl;
R³ is the same as defined for ULM-o and ULM-p
one of R^{14a} and R^{14b} is H, optionally substituted alkyl, optionally substituted haloalkyl, optionally substituted alkoxy, optionally substituted hydroxyl alkyl, optionally substituted alkylamine, optionally substituted amide, optionally substituted alkyl-amide, optionally substituted alkyl-cyano, optionally substituted alkyl-phosphate, optionally substituted heteroalkyl, optionally substituted alkyl-heterocycloalkyl, optionally substituted alkoxy-heterocycloalkyl, COR²⁶, CONR^{27a}R^{27b}, NHCOR²⁶, or NHCH₃COR²⁶; and the other of R₁₄ₐ and R^{14b} is H; or R^{14a} and R^{14b}, together with the carbon atom to which they are attached, form an optionally substituted 3 to 5 membered cycloalkyl, heterocycloalkyl, spirocycloalkyl or spiroheterocyclyl, wherein the spiroheterocyclyl is not epoxide or aziridine;
each R₂₇ₐ and R_{27b} is independently H C₁₋₆ alkyl or cycloalkyl (e.g., optionally substituted 3-5 member cycloalkyl);
o is 0, 1, or 2;
q is 1, 2, 3 or 4;
R¹⁵ is optionally substituted

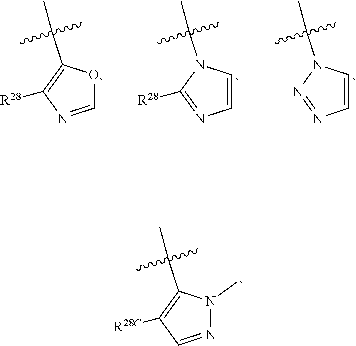

or CN;
R²⁸ is H, methyl, CH₂N(Me)₂, CH₂OH, CH₂O(C₁₋₄alkyl), CH₂NHC(O)C₁₋₄alkyl, NH₂,

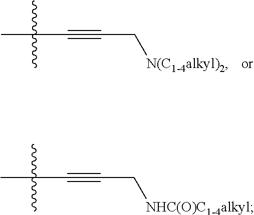

R^{28C} is H, methyl, fluoro, or chloro; and
R¹⁶ is H, C₁₋₄alkyl, fluoro, chloro, CN, or C₁₋₄alkoxy.

In any aspect or embodiment described herein, R^{14a} and R^{14b} are selected from: H, C₁₋₄ alkyl, C₁₋₄ cycloalkyl, C₁₋₄haloalkyl, C₁₋₄ hydroxyalkyl, C₁₋₄ alkyloxyalkyl, C₁₋₄ alkyl-NR₂₇ₐR_{27b} and CONR₂₇ₐR_{27b}.

In any aspect or embodiment described herein, at least one of R^{14a} and R^{14b} is H (e.g., both R^{14a} and R^{14b} are H).

In any aspect or embodiment described herein, at least one of R^{14a} and R^{14b} is optionally substituted alkyl, optionally substituted haloalkyl, optionally substituted alkoxy, optionally substituted hydroxyl alkyl, optionally substituted alkylamine, optionally substituted heteroalkyl, optionally substituted alkyl-heterocycloalkyl, optionally substituted alkoxy-heterocycloalkyl, COR²⁶, CONR^{27a}R^{27b}, NHCOR²⁶, or NHCH₃COR²⁶. Alternatively, in any aspect or embodiment described herein, one of R^{14a} and R^{14b} is optionally substituted alkyl, optionally substituted haloalkyl, optionally substituted alkoxy, optionally substituted hydroxyl alkyl, optionally substituted alkylamine, optionally substituted heteroalkyl, optionally substituted alkyl-heterocycloalkyl, optionally substituted alkoxy-heterocycloalkyl, COR²⁶, CONR^{27a}R^{27b}, NHCOR²⁶, or NHCH₃COR²⁶; and the other of R^{14a} and R^{14b} is H.

In any aspect or embodiment described herein, R^{14a} and R^{14b} together with the carbon atom to which they are attached form

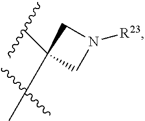

wherein R²³ is selected from H, C₁₋₄alkyl, —C(O)C₁₋₄alkyl.

In other preferred embodiments of the disclosure, ULM and where present, ULM′, are each independently a group according to the chemical structure:

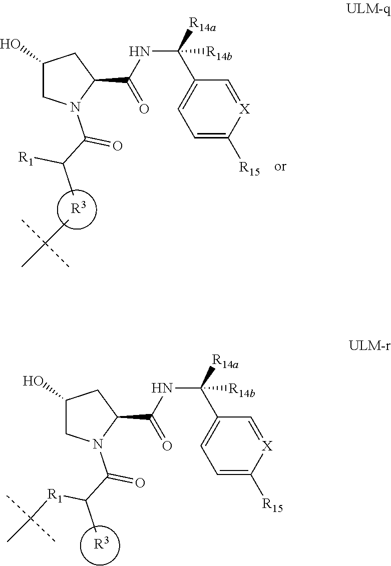

or a pharmaceutically acceptable salt thereof, wherein:
X is CH or N; and
R₁, R₃, R₁₄ₐ, R_{14b}, and R₁₅ of ULM-q and ULM-r are the same as defined for ULM-o and ULM-p.

In any aspect or embodiment described herein, one of R₁₄ₐ and R_{14b} is H, optionally substituted alkyl, haloalkyl, optionally substituted alkoxy, optionally substituted hydroxyl alkyl, optionally substituted alkylamine, optionally substituted amide, optionally substituted alkyl-amide, optionally substituted alkyl-cyano, optionally substituted heteroalkyl, or optionally substituted alkyl-heterocycloalkyl, and the other of R₁₄ₐ and R_{14b} is H; or R₁₄ₐ, R_{14b}, together with the carbon atom to which they are attached, form an optionally substituted 3 to 5 membered cycloalkyl, heterocycloalkyl, spirocycloalkyl or spiroheterocyclyl, wherein the spiroheterocyclyl is not epoxide or aziridine.

In any aspect or embodiment described herein, one of R₁₄ₐ and R_{14b} is H, optionally substituted alkyl, haloalkyl, optionally substituted alkoxy, optionally substituted hydroxyl alkyl, optionally substituted alkylamine, optionally substituted amide, optionally substituted alkyl-amide, optionally substituted alkyl-cyano, optionally substituted heteroalkyl, or optionally substituted alkyl-heterocycloalkyl, and the other of R₁₄ₐ and R_{14b} is H.

In any aspect or embodiment described herein, one R₁₄ₐ and R_{14b} are selected from: H, C₁₋₄ alkyl, C₃₋₄ cycloalkyl, C₁₋₄ haloalkyl, C₁₋₄hydroxyalkyl, and C₁₋₄ alkyloxyalkyl; and one of R₁₄ₐ and R_{14b} is H.

In any aspect or embodiment described herein, one of R₁₄ₐ and R_{14b} is H, C₁₋₆ alkyl, C₁₋₆ haloalkyl, optionally substitute C₁₋₄ alkylamine, C₁₋₆ alkoxy, (CH₂)_{q}C₁₋₆ alkoxy, (CH₂)_{q}C₁₋₆ alkoxy-C₃₋₇ heterocycloalkyl, (CH₂)_{q}OH, (CH₂)_{q}NR₂₇ₐR_{27b}, C₃₋₆ cycloalkyl, or NR₂₇ₐR_{27b}; and one of R₁₄ₐ and R_{14b} is H.

In any of the aspects or embodiments described herein, the ULM (or when present, ULM′) as described herein may be a pharmaceutically acceptable salt, enantiomer, diastereomer, solvate or polymorph thereof. In addition, in any of the aspects or embodiments described herein, the ULM (or when present, ULM′) as described herein may be coupled to a PTM directly via a bond or by a chemical linker.

In certain aspects of the disclosure, the ULM moiety is selected from the group consisting of:

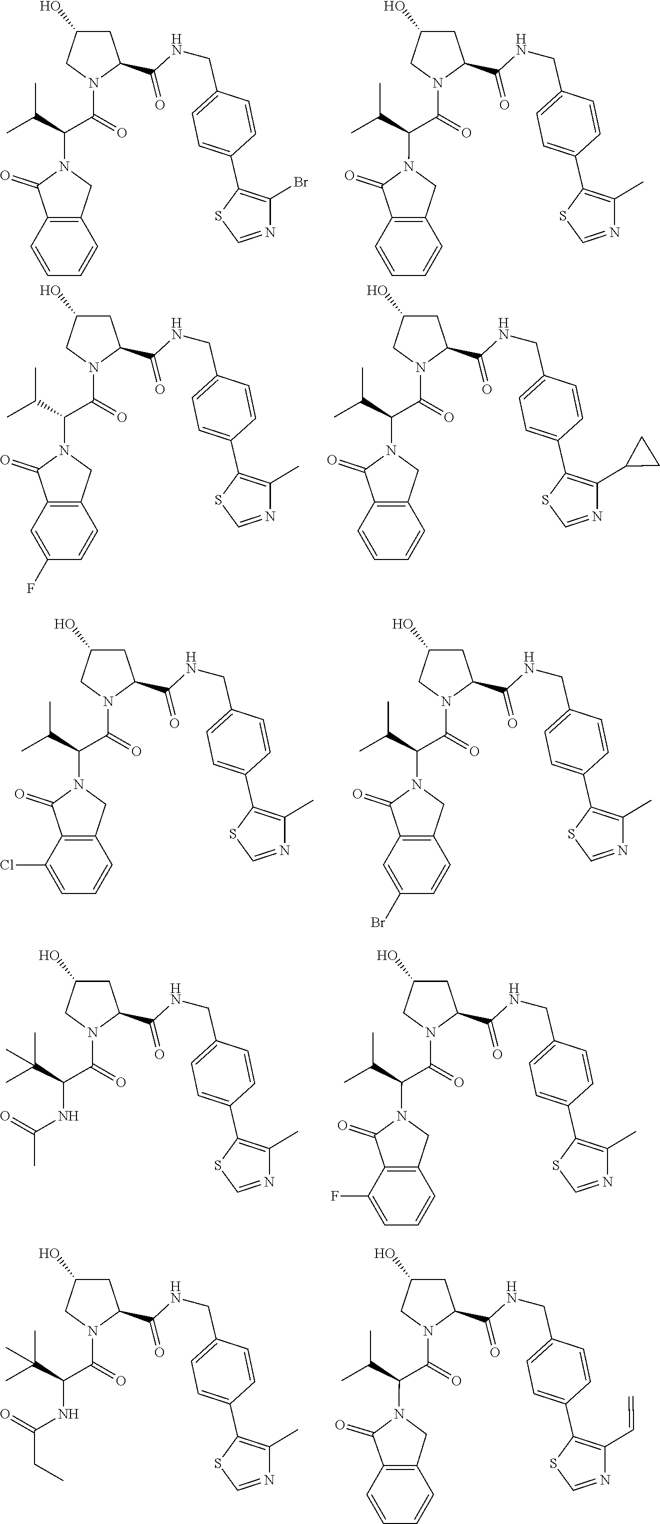

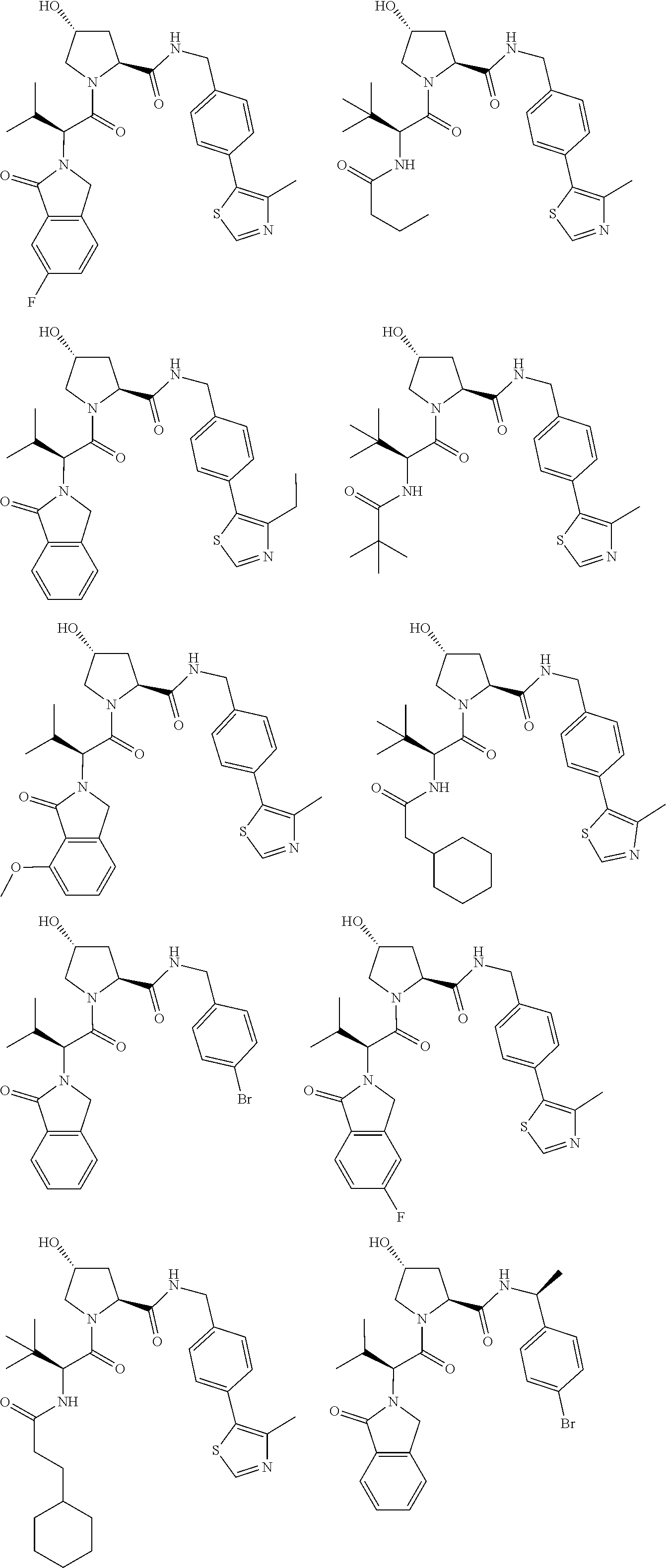

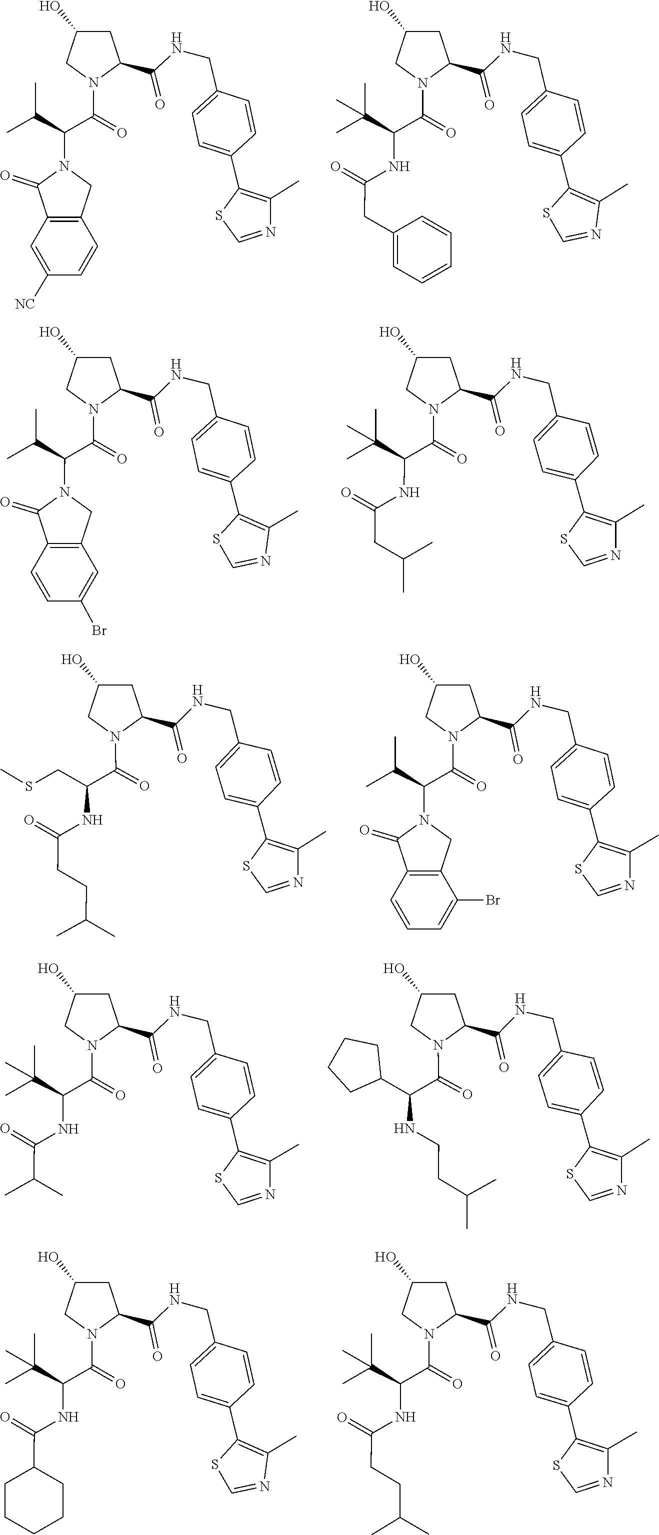

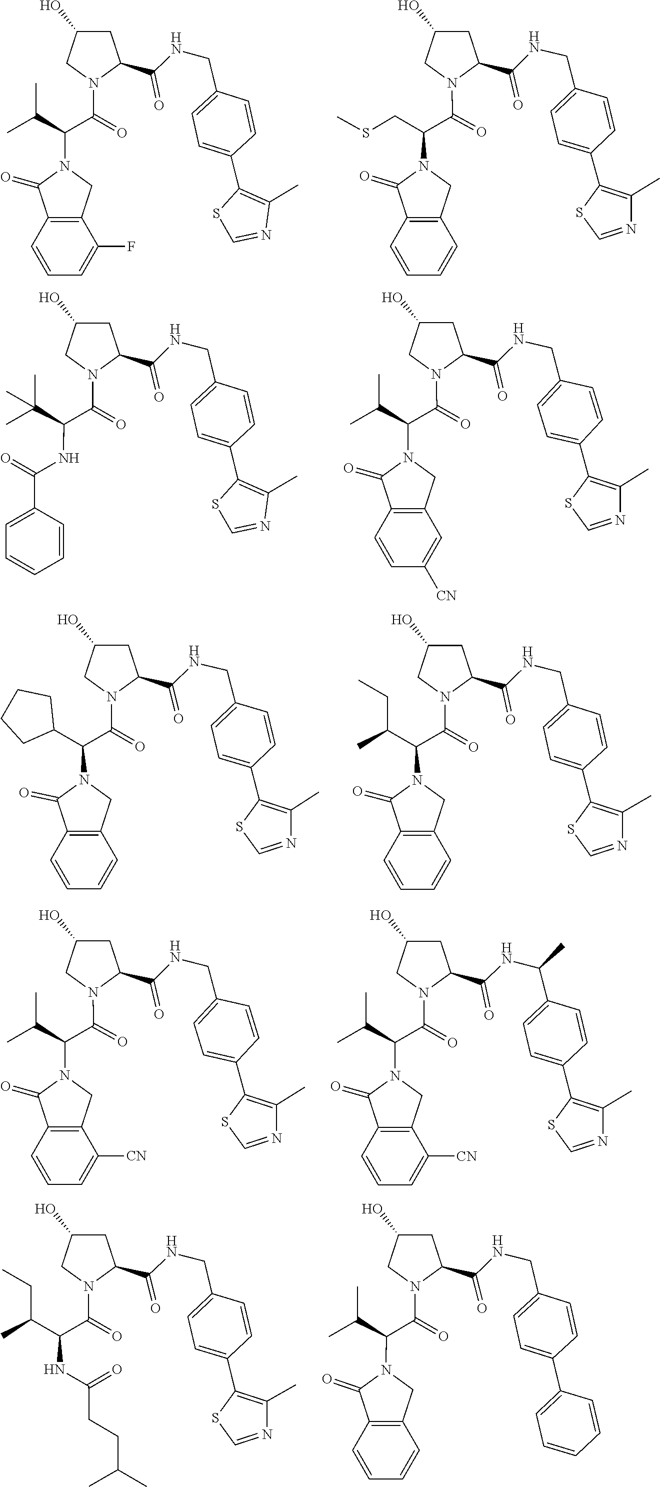

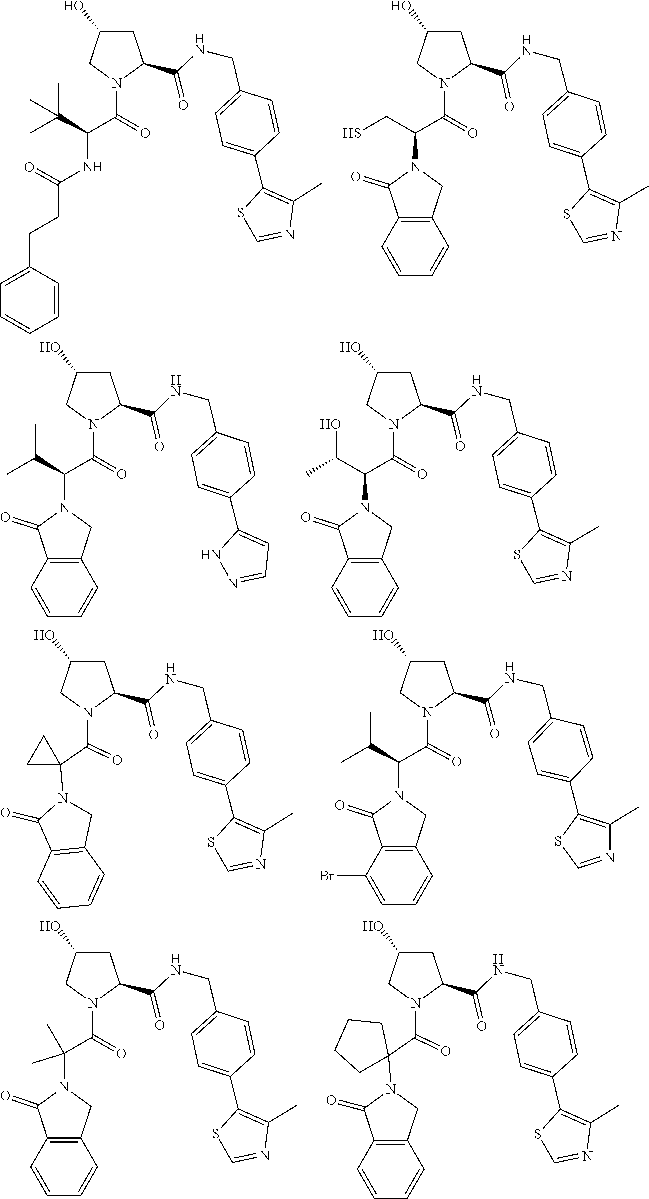

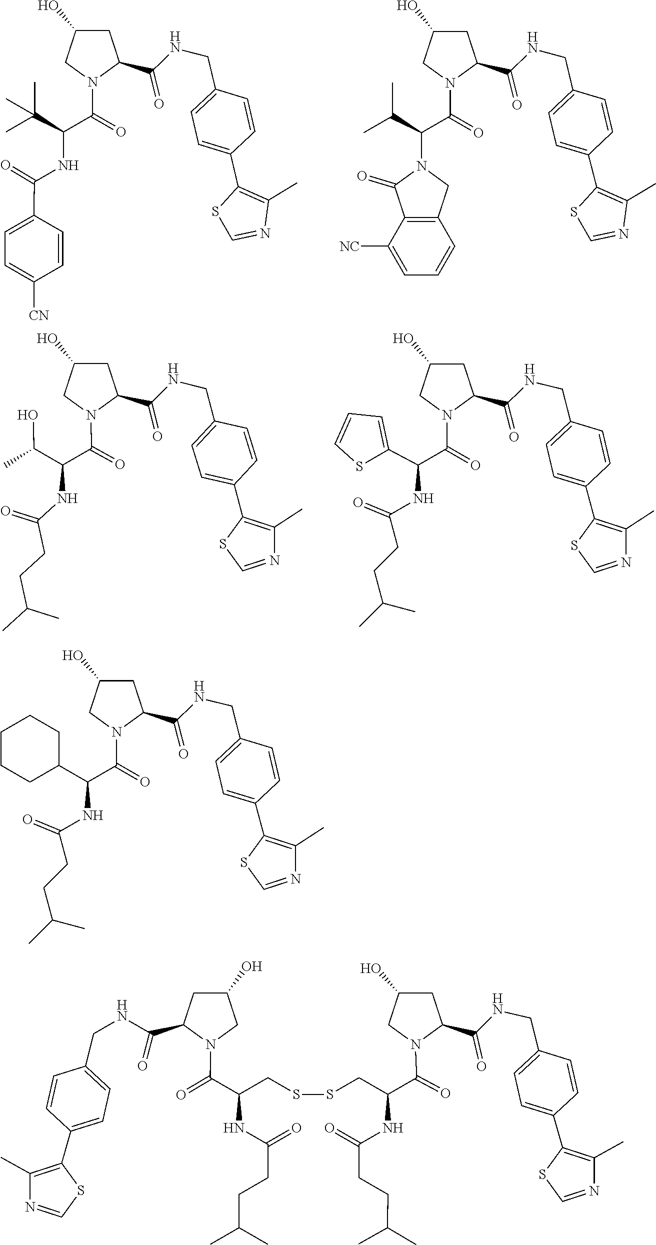

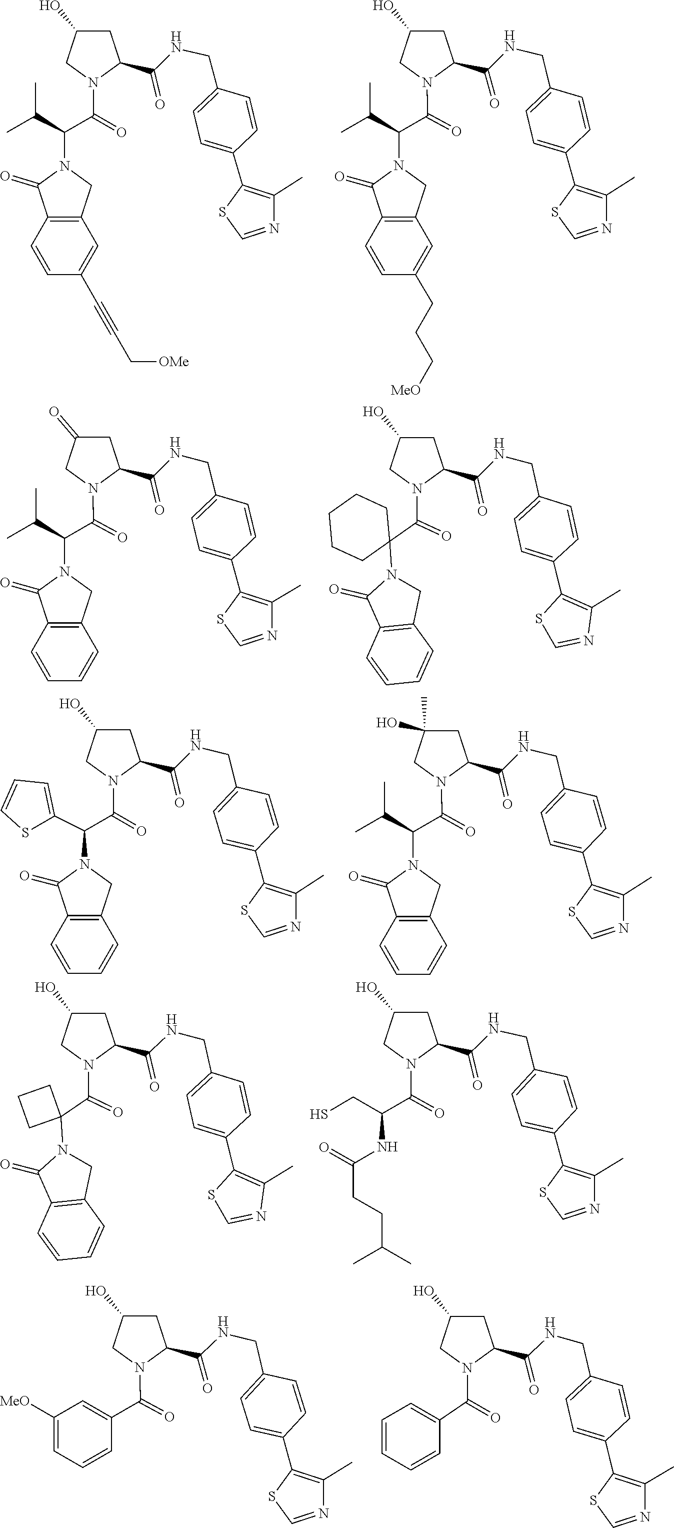

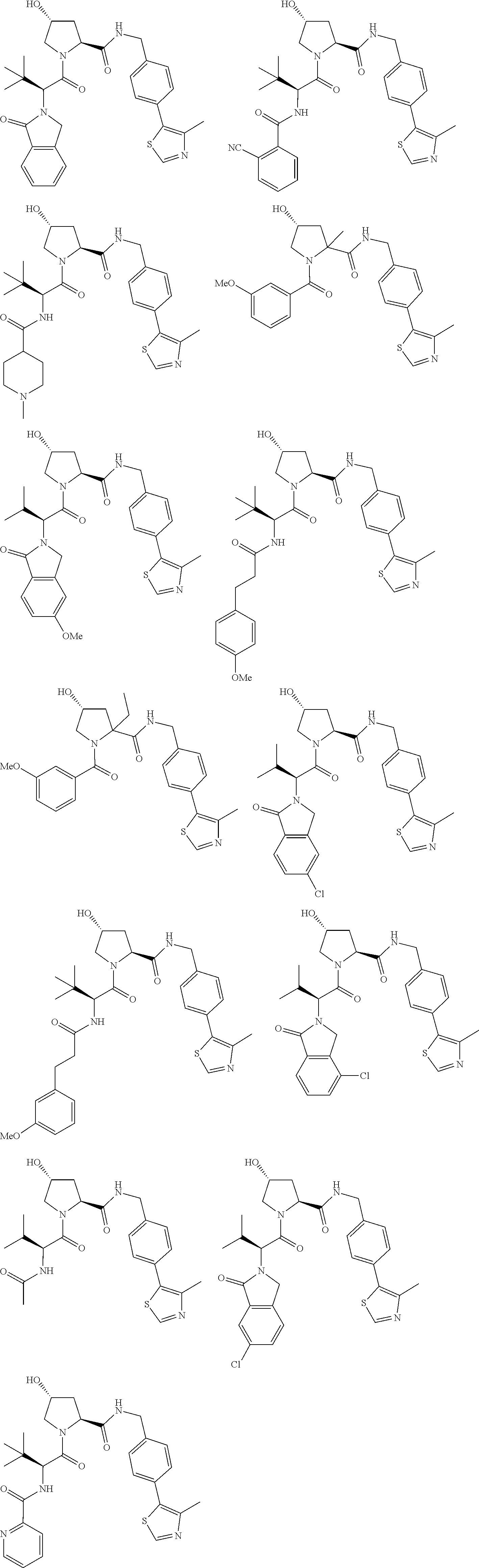

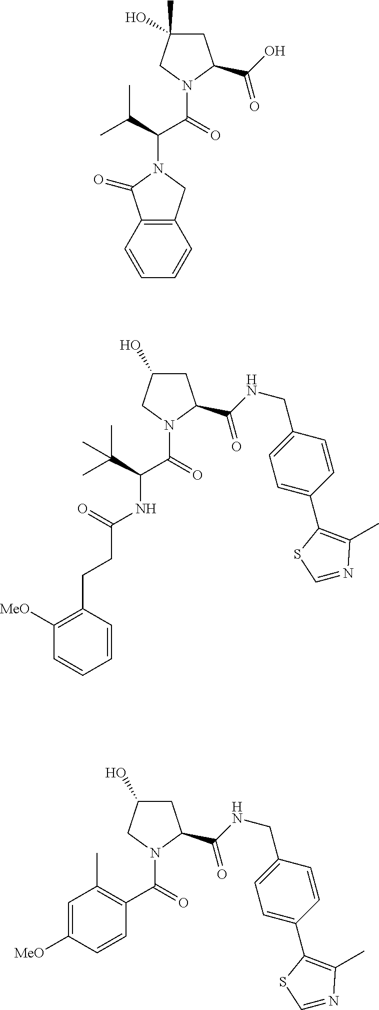 [SMILES: CC(C)[C@@H](C(=O)N1C[C@](C)(O)C[C@H]1C(=O)O)N1Cc2ccccc2C1=O ; COc1ccccc1CCC(=O)N[C@H](C(=O)N1C[C@H](O)C[C@H]1C(=O)NCc1ccc(-c2scnc2C)cc1)C(C)(C)C ; COc1ccc(C(=O)N2C[C@H](O)C[C@H]2C(=O)NCc2ccc(-c3scnc3C)cc2)c(C)c1]

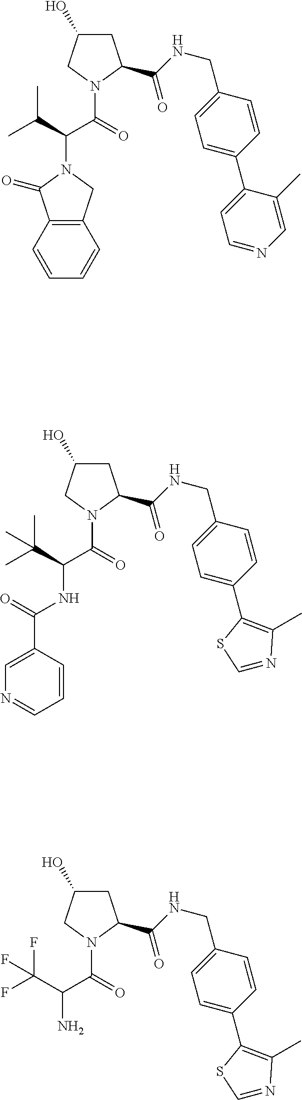

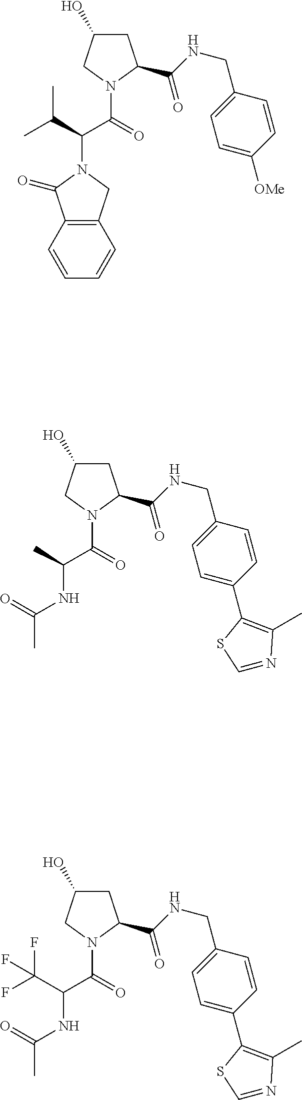 [SMILES: COc1ccc(CNC(=O)[C@@H]2C[C@@H](O)CN2C(=O)[C@H](C(C)C)N2Cc3ccccc3C2=O)cc1 ; CC(=O)N[C@@H](C)C(=O)N1C[C@H](O)C[C@H]1C(=O)NCc1ccc(-c2scnc2C)cc1 ; CC(=O)NC(C(=O)N1C[C@H](O)C[C@H]1C(=O)NCc1ccc(-c2scnc2C)cc1)C(F)(F)F]

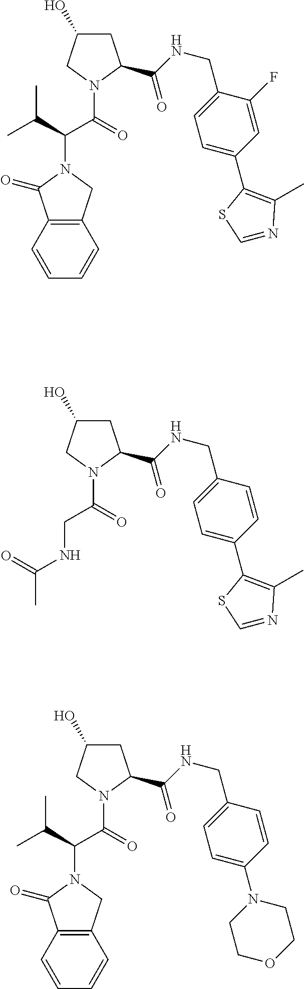 [SMILES: Cc1ncsc1-c1ccc(CNC(=O)[C@@H]2C[C@@H](O)CN2C(=O)[C@H](C(C)C)N2Cc3ccccc3C2=O)c(F)c1 ; CC(=O)NCC(=O)N1C[C@H](O)C[C@H]1C(=O)NCc1ccc(-c2scnc2C)cc1 ; CC(C)[C@@H](C(=O)N1C[C@H](O)C[C@H]1C(=O)NCc1ccc(N2CCOCC2)cc1)N1Cc2ccccc2C1=O]

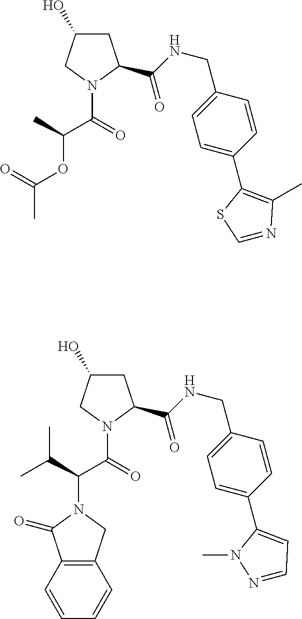 [SMILES: CC(=O)O[C@@H](C)C(=O)N1C[C@H](O)C[C@H]1C(=O)NCc1ccc(-c2scnc2C)cc1 ; CC(C)[C@@H](C(=O)N1C[C@H](O)C[C@H]1C(=O)NCc1ccc(-c2ccnn2C)cc1)N1Cc2ccccc2C1=O]

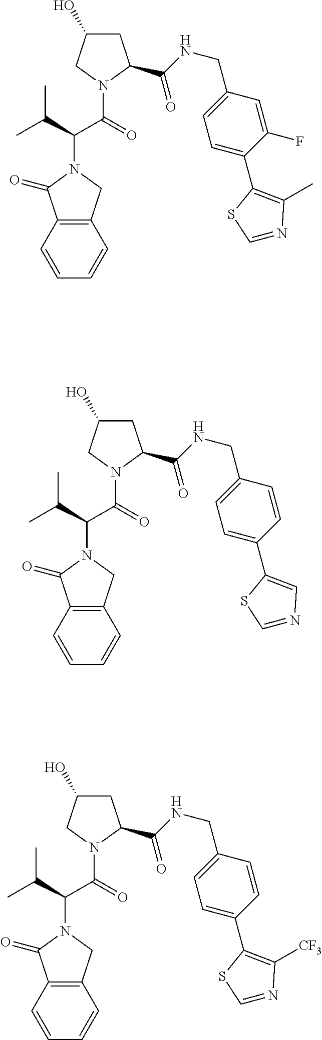 [SMILES: Cc1ncsc1-c1ccc(CNC(=O)[C@@H]2C[C@@H](O)CN2C(=O)[C@H](C(C)C)N2Cc3ccccc3C2=O)cc1F ; CC(C)[C@@H](C(=O)N1C[C@H](O)C[C@H]1C(=O)NCc1ccc(-c2cncs2)cc1)N1Cc2ccccc2C1=O ; CC(C)[C@@H](C(=O)N1C[C@H](O)C[C@H]1C(=O)NCc1ccc(-c2scnc2C(F)(F)F)cc1)N1Cc2ccccc2C1=O]

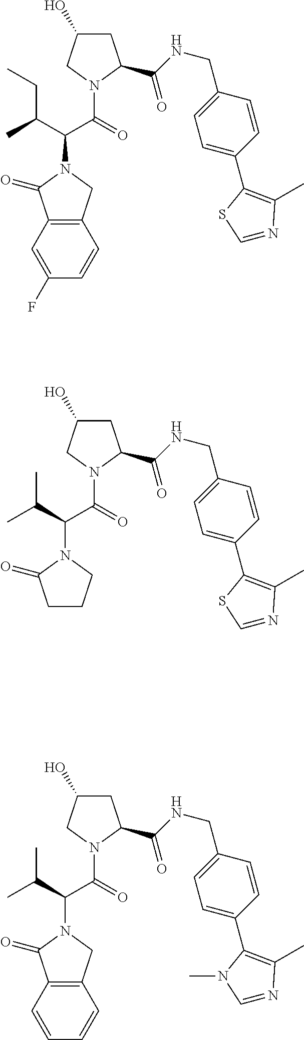 [SMILES: CC[C@H](C)[C@@H](C(=O)N1C[C@H](O)C[C@H]1C(=O)NCc1ccc(-c2scnc2C)cc1)N1Cc2ccc(F)cc2C1=O ; Cc1ncsc1-c1ccc(CNC(=O)[C@@H]2C[C@@H](O)CN2C(=O)[C@H](C(C)C)N2CCCC2=O)cc1 ; Cc1ncn(C)c1-c1ccc(CNC(=O)[C@@H]2C[C@@H](O)CN2C(=O)[C@H](C(C)C)N2Cc3ccccc3C2=O)cc1]

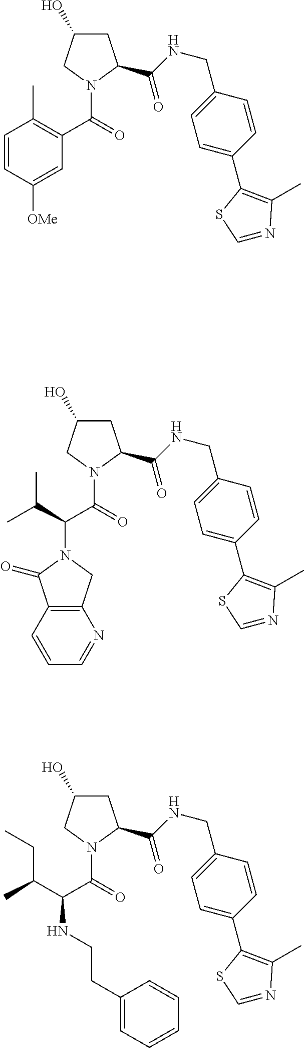

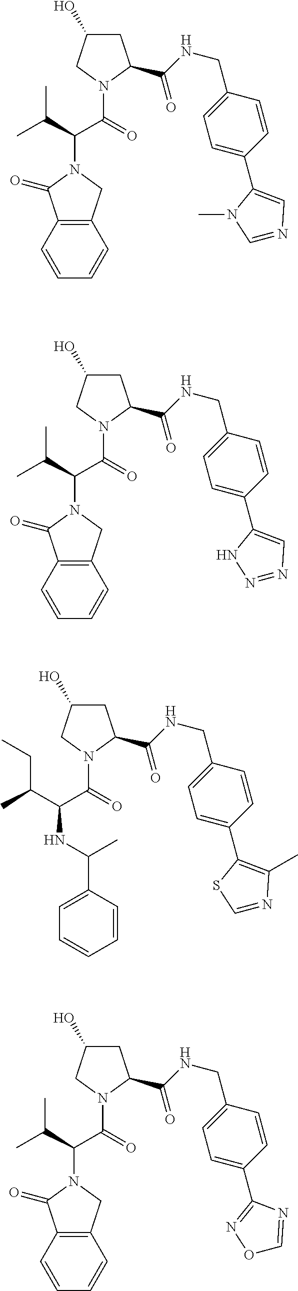 [SMILES: CC(C)[C@@H](C(=O)N1C[C@H](O)C[C@H]1C(=O)NCc1ccc(-c2cncn2C)cc1)N1Cc2ccccc2C1=O ; CC(C)[C@@H](C(=O)N1C[C@H](O)C[C@H]1C(=O)NCc1ccc(-c2cnn[nH]2)cc1)N1Cc2ccccc2C1=O ; CC[C@H](C)[C@H](NC(C)c1ccccc1)C(=O)N1C[C@H](O)C[C@H]1C(=O)NCc1ccc(-c2scnc2C)cc1 ; CC(C)[C@@H](C(=O)N1C[C@H](O)C[C@H]1C(=O)NCc1ccc(-c2ncon2)cc1)N1Cc2ccccc2C1=O]

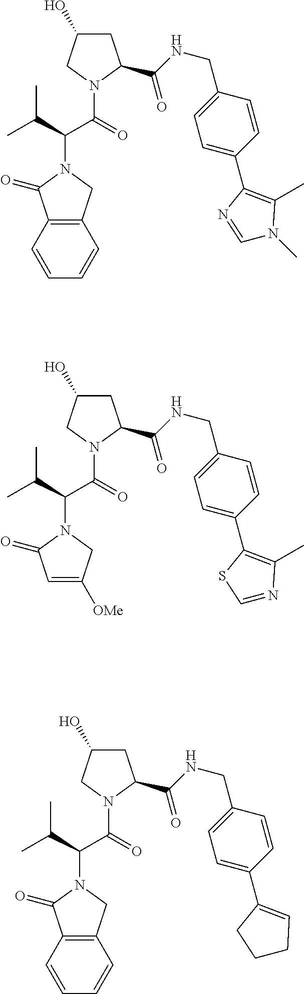 [SMILES: Cc1c(-c2ccc(CNC(=O)[C@@H]3C[C@@H](O)CN3C(=O)[C@H](C(C)C)N3Cc4ccccc4C3=O)cc2)ncn1C ; COC1=CC(=O)N([C@H](C(=O)N2C[C@H](O)C[C@H]2C(=O)NCc2ccc(-c3scnc3C)cc2)C(C)C)C1 ; CC(C)[C@@H](C(=O)N1C[C@H](O)C[C@H]1C(=O)NCc1ccc(C2=CCCC2)cc1)N1Cc2ccccc2C1=O]

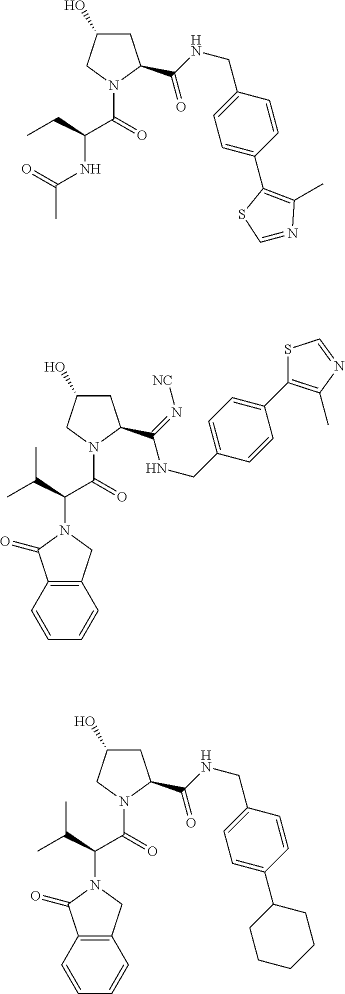 [SMILES: CC[C@H](NC(C)=O)C(=O)N1C[C@H](O)C[C@H]1C(=O)NCc1ccc(-c2scnc2C)cc1 ; Cc1ncsc1-c1ccc(CN/C(=N/C#N)[C@@H]2C[C@@H](O)CN2C(=O)[C@H](C(C)C)N2Cc3ccccc3C2=O)cc1 ; CC(C)[C@@H](C(=O)N1C[C@H](O)C[C@H]1C(=O)NCc1ccc(C2CCCCC2)cc1)N1Cc2ccccc2C1=O]

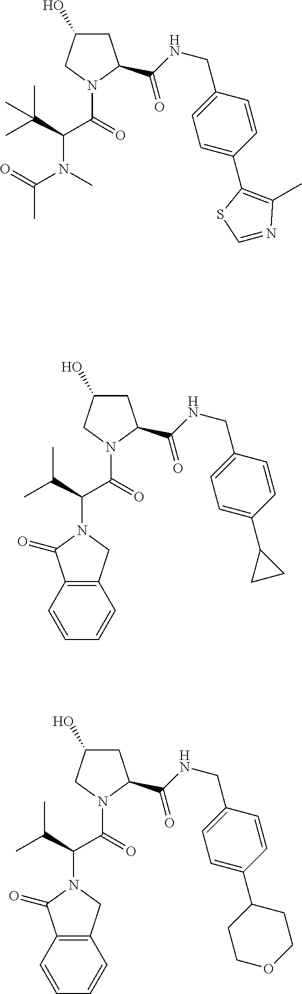 [SMILES: CC(=O)N(C)[C@H](C(=O)N1C[C@H](O)C[C@H]1C(=O)NCc1ccc(-c2scnc2C)cc1)C(C)(C)C ; CC(C)[C@@H](C(=O)N1C[C@H](O)C[C@H]1C(=O)NCc1ccc(C2CC2)cc1)N1Cc2ccccc2C1=O ; CC(C)[C@@H](C(=O)N1C[C@H](O)C[C@H]1C(=O)NCc1ccc(C2CCOCC2)cc1)N1Cc2ccccc2C1=O]

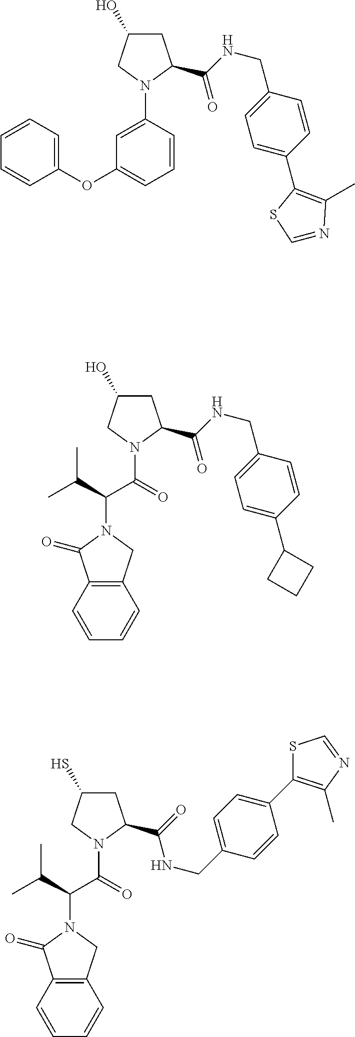 [SMILES: Cc1ncsc1-c1ccc(CNC(=O)[C@@H]2C[C@@H](O)CN2c2cccc(Oc3ccccc3)c2)cc1 ; CC(C)[C@@H](C(=O)N1C[C@H](O)C[C@H]1C(=O)NCc1ccc(C2CCC2)cc1)N1Cc2ccccc2C1=O ; Cc1ncsc1-c1ccc(CNC(=O)[C@@H]2C[C@@H](S)CN2C(=O)[C@H](C(C)C)N2Cc3ccccc3C2=O)cc1]

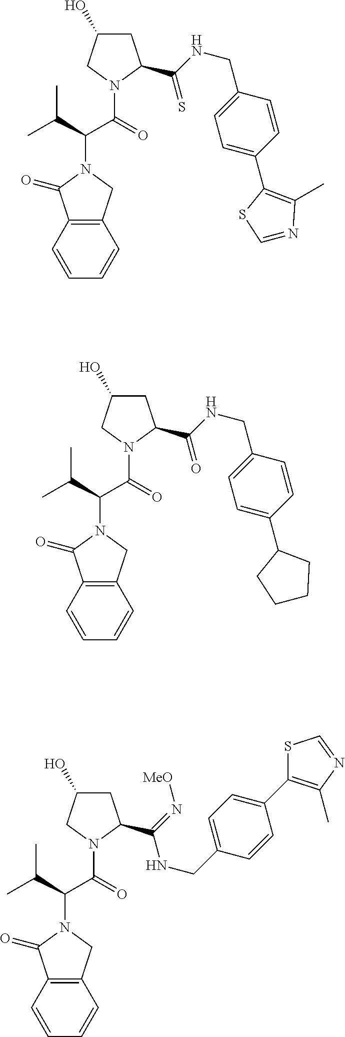 [SMILES: Cc1ncsc1-c1ccc(CNC(=S)[C@@H]2C[C@@H](O)CN2C(=O)[C@H](C(C)C)N2Cc3ccccc3C2=O)cc1 ; CC(C)[C@@H](C(=O)N1C[C@H](O)C[C@H]1C(=O)NCc1ccc(C2CCCC2)cc1)N1Cc2ccccc2C1=O ; CO/N=C(/NCc1ccc(-c2scnc2C)cc1)[C@@H]1C[C@@H](O)CN1C(=O)[C@H](C(C)C)N1Cc2ccccc2C1=O]

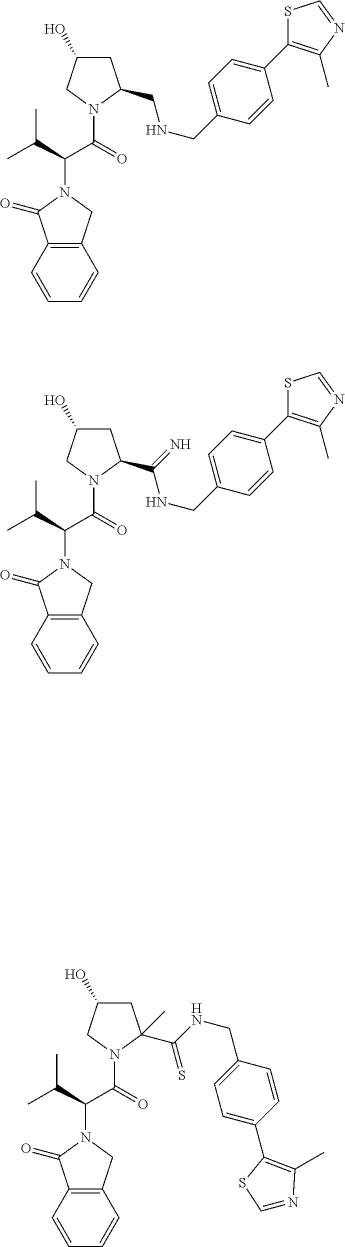 [SMILES: Cc1ncsc1-c1ccc(CNC[C@@H]2C[C@@H](O)CN2C(=O)[C@H](C(C)C)N2Cc3ccccc3C2=O)cc1 ; Cc1ncsc1-c1ccc(CNC(=N)[C@@H]2C[C@@H](O)CN2C(=O)[C@H](C(C)C)N2Cc3ccccc3C2=O)cc1 ; Cc1ncsc1-c1ccc(CNC(=S)C2(C)C[C@@H](O)CN2C(=O)[C@H](C(C)C)N2Cc3ccccc3C2=O)cc1]

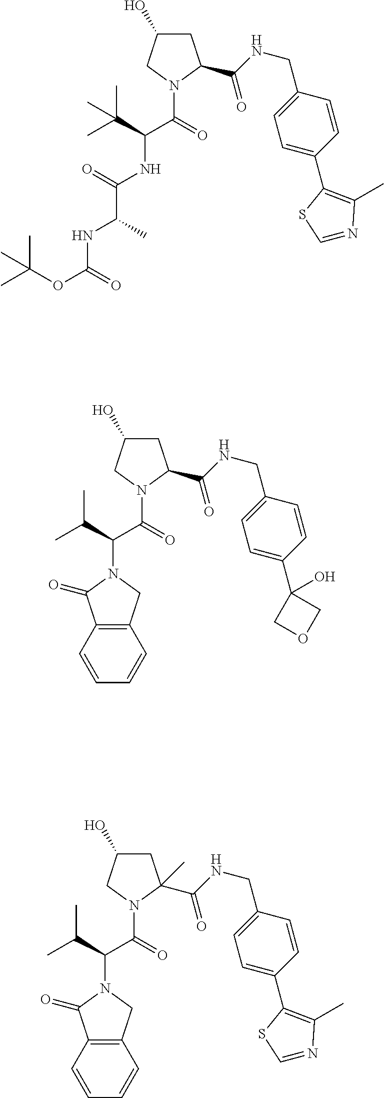 [SMILES: Cc1ncsc1-c1ccc(CNC(=O)[C@@H]2C[C@@H](O)CN2C(=O)[C@@H](NC(=O)[C@H](C)NC(=O)OC(C)(C)C)C(C)(C)C)cc1 ; CC(C)[C@@H](C(=O)N1C[C@H](O)C[C@H]1C(=O)NCc1ccc(C2(O)COC2)cc1)N1Cc2ccccc2C1=O ; Cc1ncsc1-c1ccc(CNC(=O)C2(C)C[C@@H](O)CN2C(=O)[C@H](C(C)C)N2Cc3ccccc3C2=O)cc1]

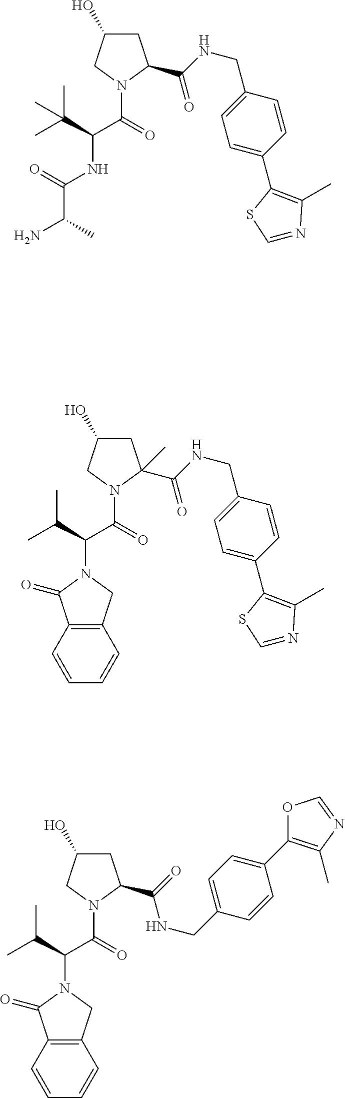 [SMILES: Cc1ncsc1-c1ccc(CNC(=O)[C@@H]2C[C@@H](O)CN2C(=O)[C@@H](NC(=O)[C@H](C)N)C(C)(C)C)cc1 ; Cc1ncsc1-c1ccc(CNC(=O)C2(C)C[C@@H](O)CN2C(=O)[C@H](C(C)C)N2Cc3ccccc3C2=O)cc1 ; Cc1ncoc1-c1ccc(CNC(=O)[C@@H]2C[C@@H](O)CN2C(=O)[C@H](C(C)C)N2Cc3ccccc3C2=O)cc1]

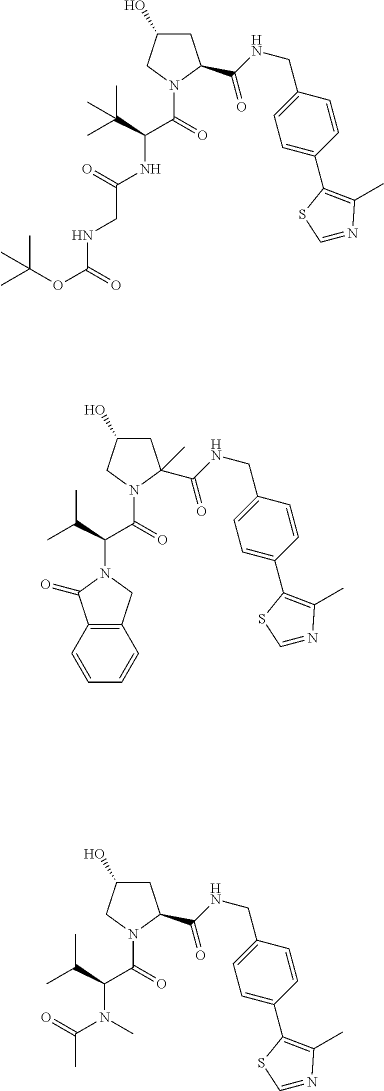

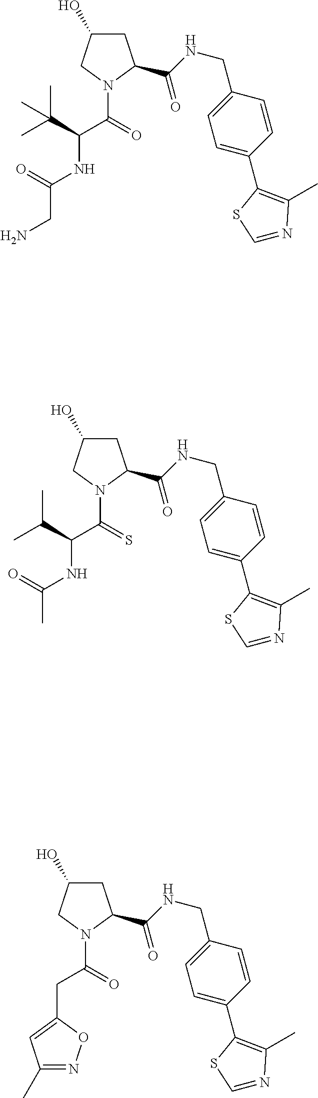

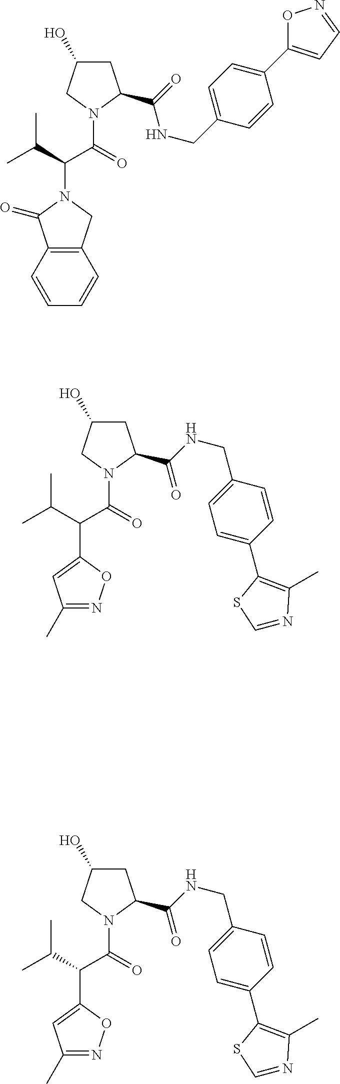

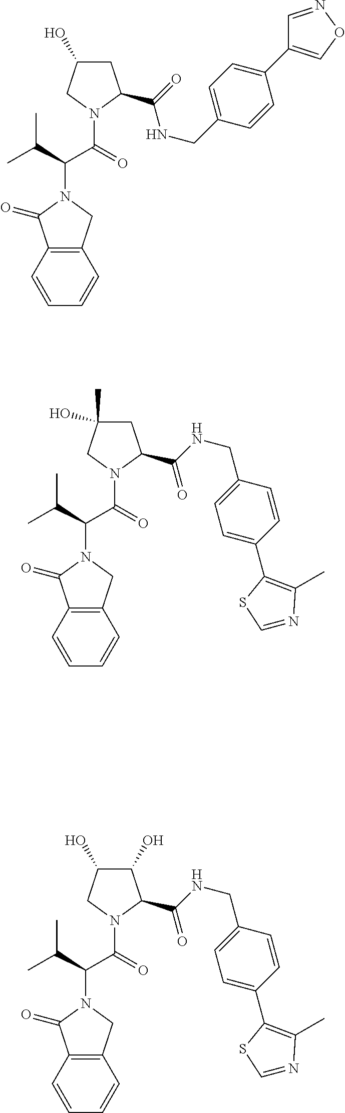 [SMILES: CC(C)[C@@H](C(=O)N1C[C@H](O)C[C@H]1C(=O)NCc1ccc(-c2cnoc2)cc1)N1Cc2ccccc2C1=O ; Cc1ncsc1-c1ccc(CNC(=O)[C@@H]2C[C@@](C)(O)CN2C(=O)[C@H](C(C)C)N2Cc3ccccc3C2=O)cc1 ; Cc1ncsc1-c1ccc(CNC(=O)[C@@H]2[C@@H](O)[C@@H](O)CN2C(=O)[C@H](C(C)C)N2Cc3ccccc3C2=O)cc1]

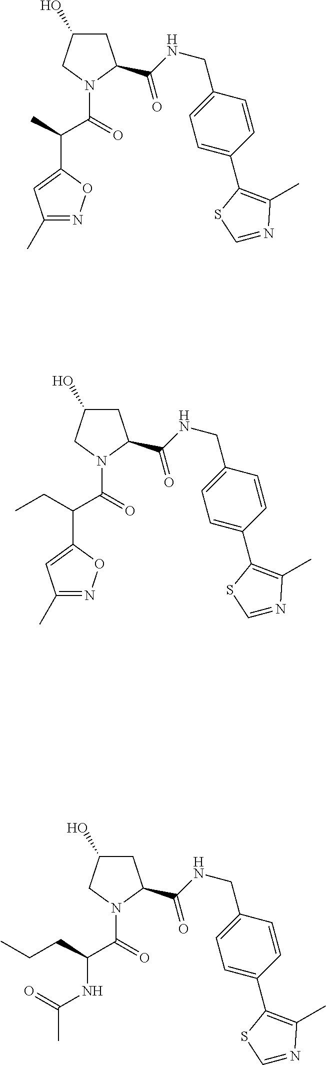

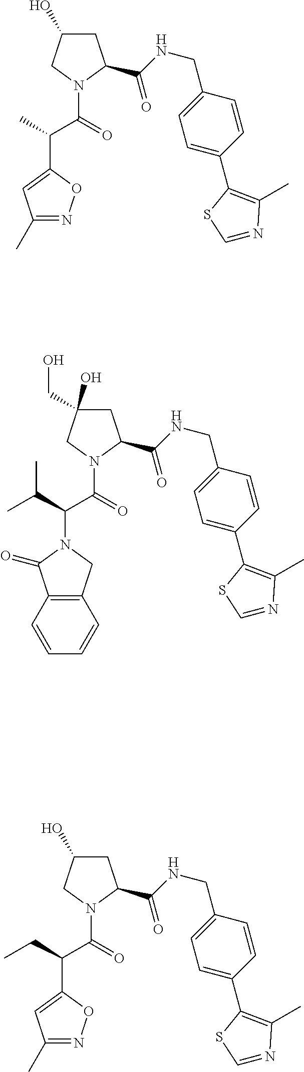

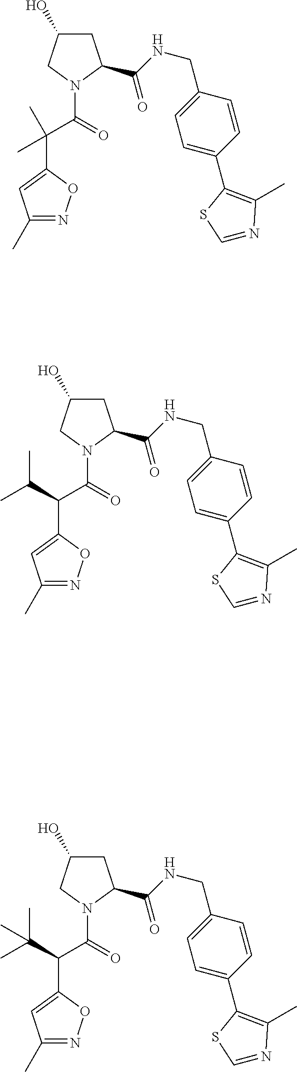

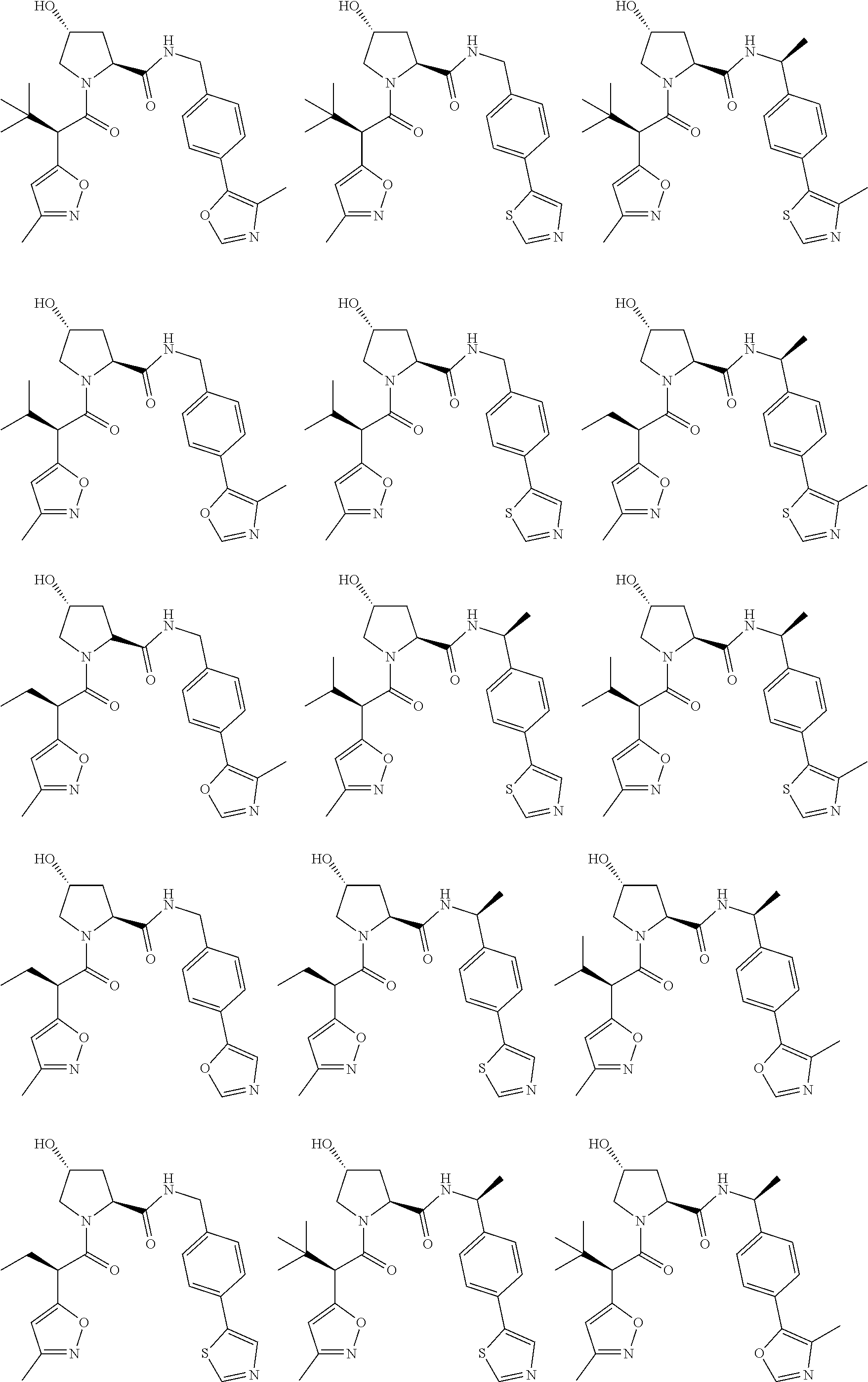

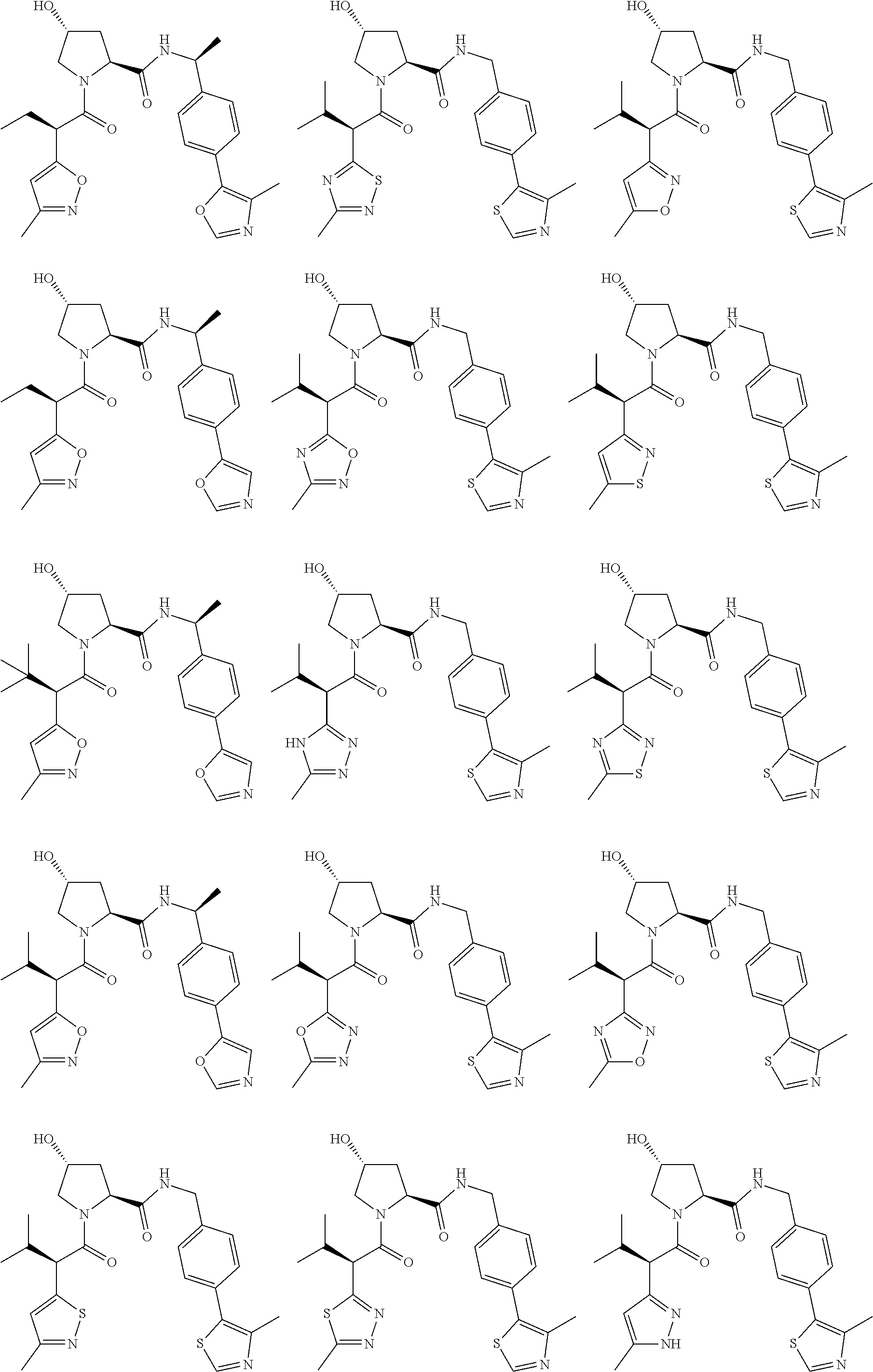

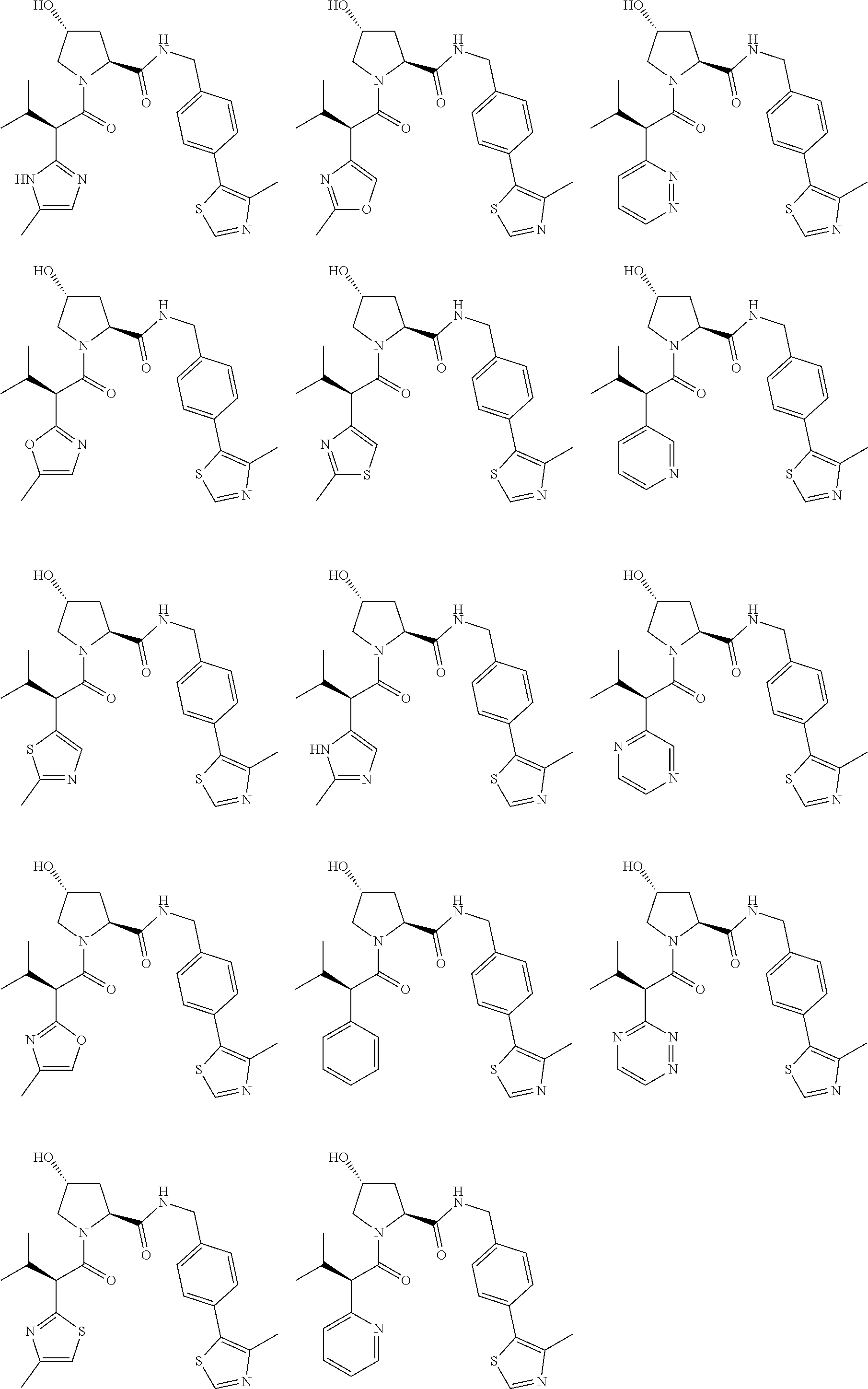

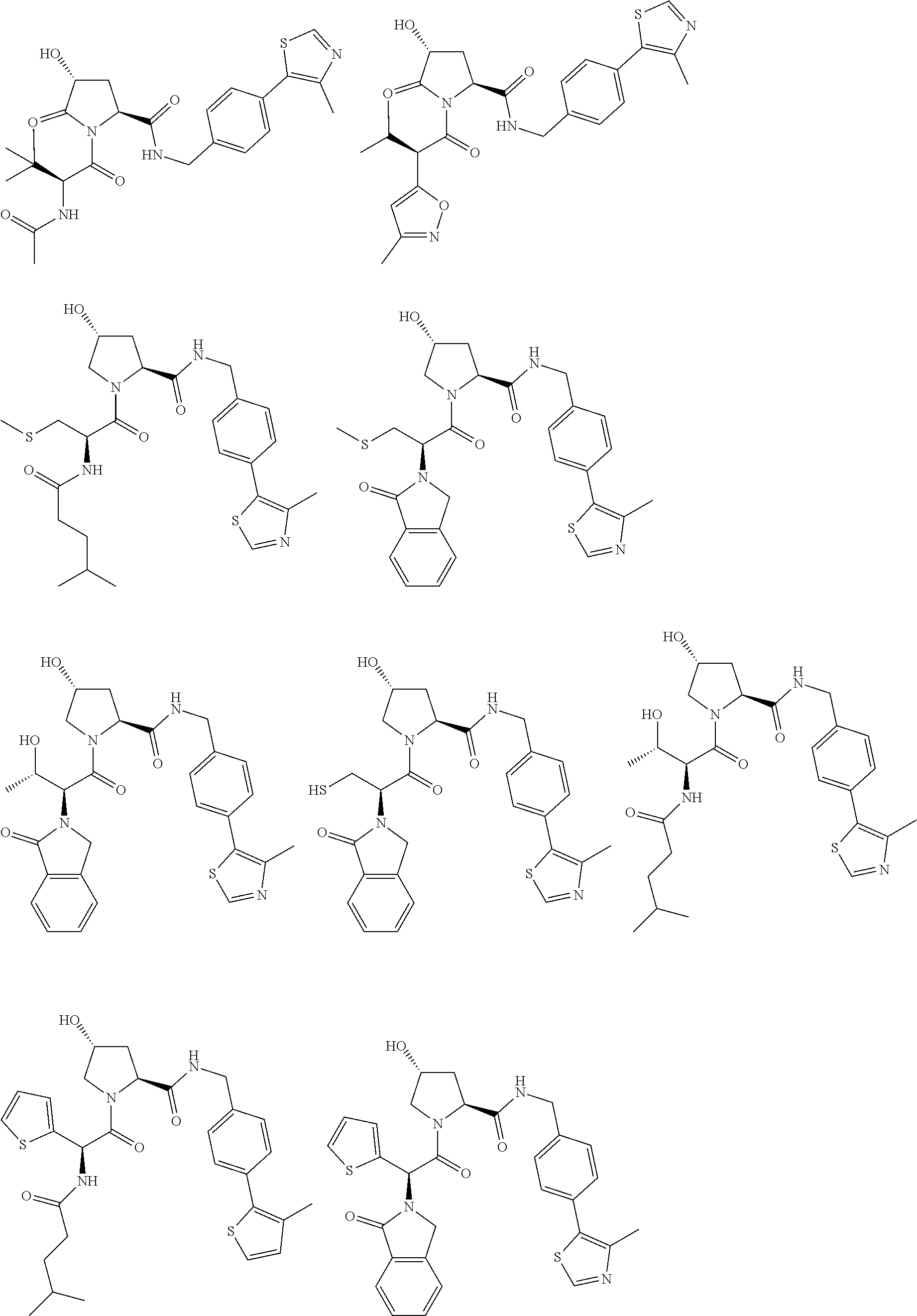

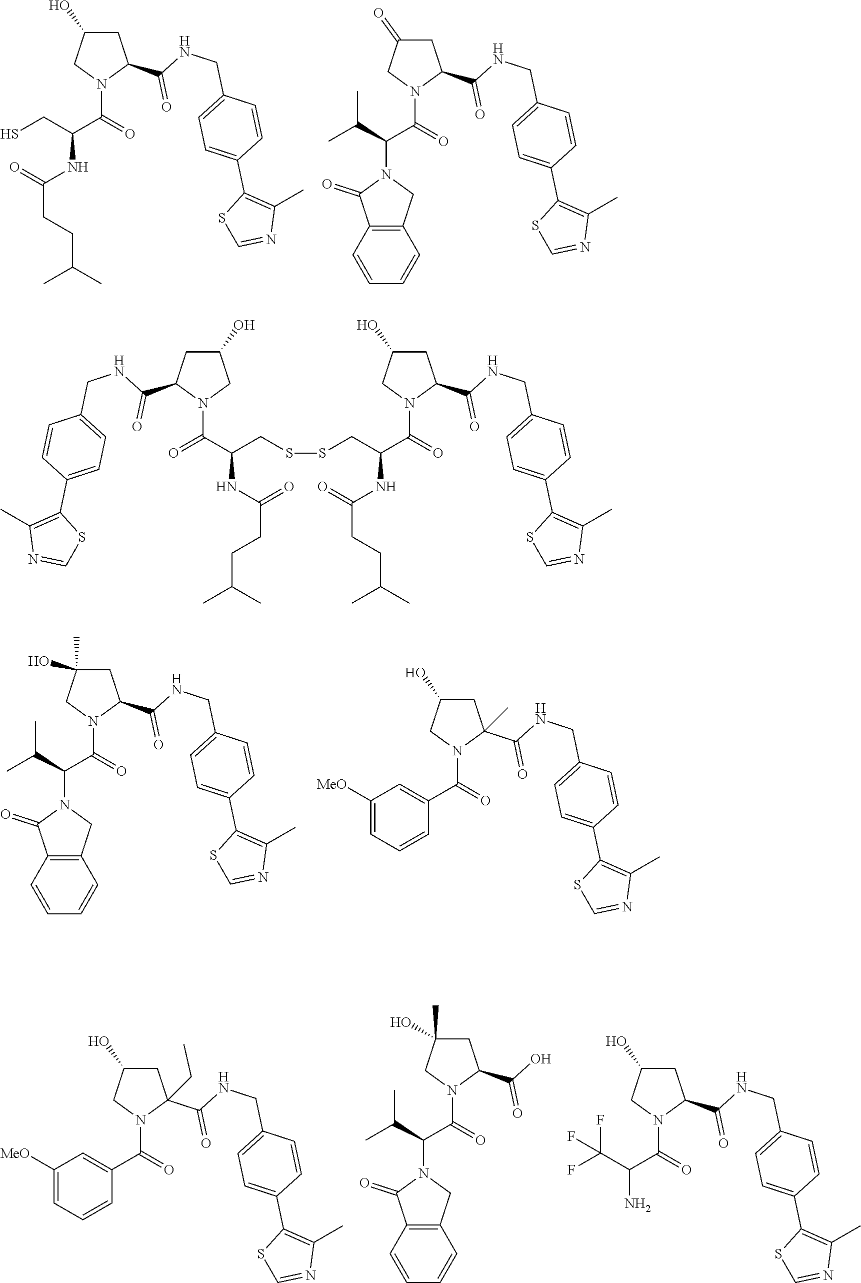

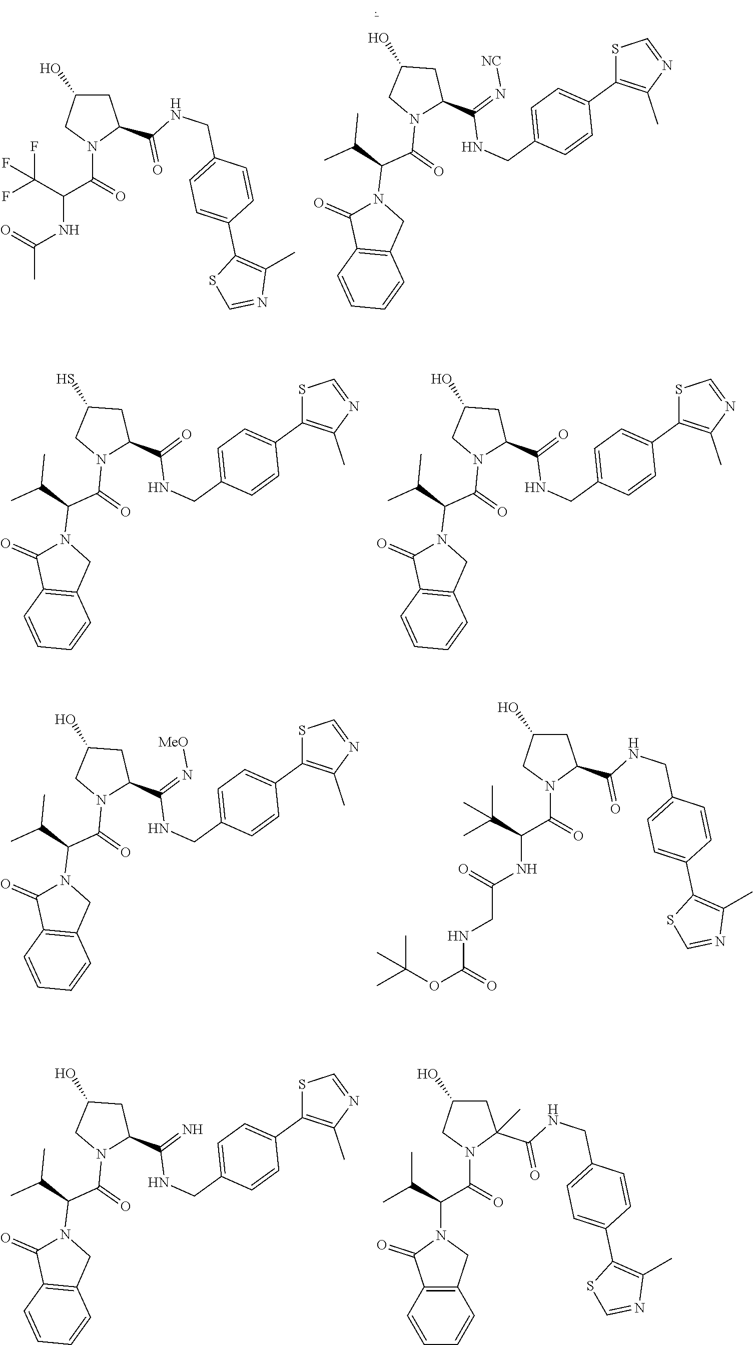

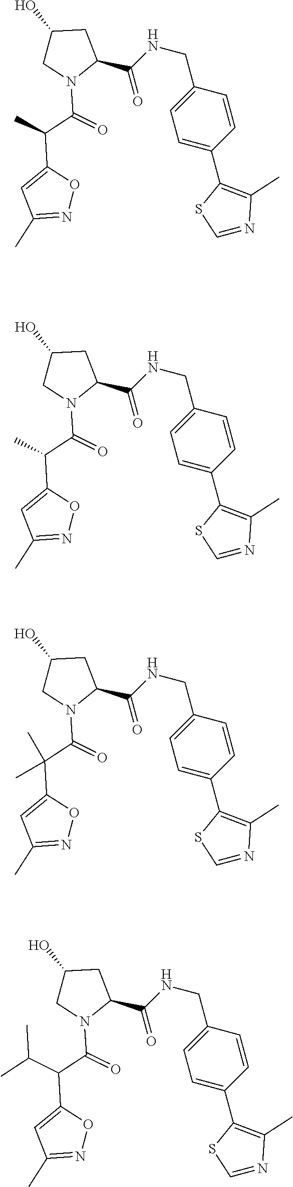

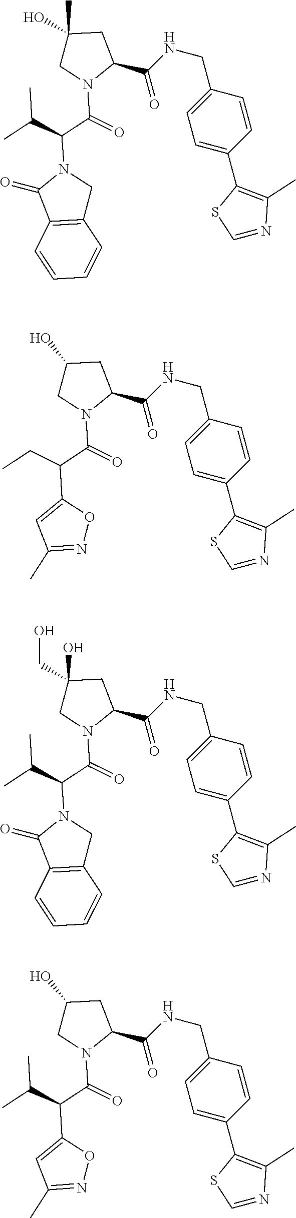

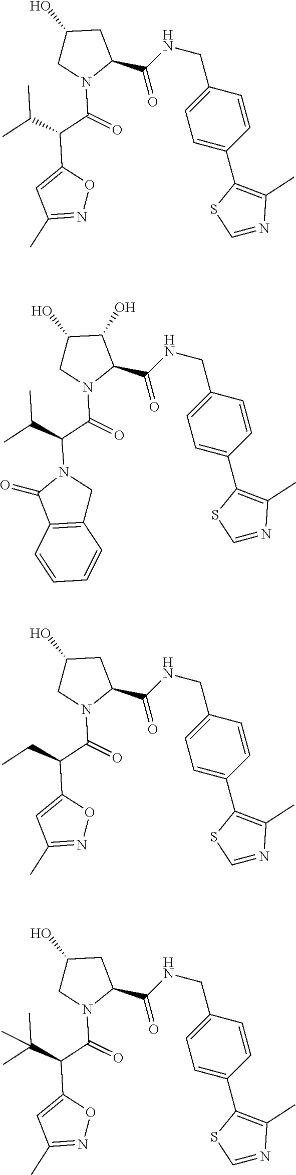

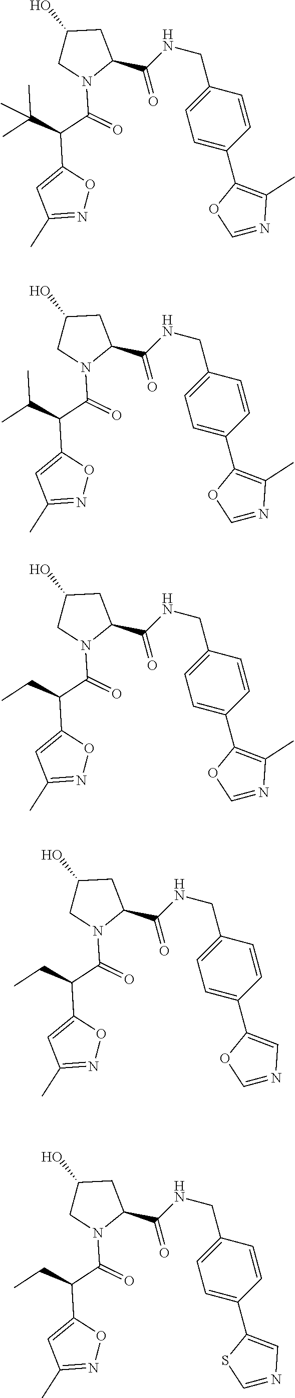

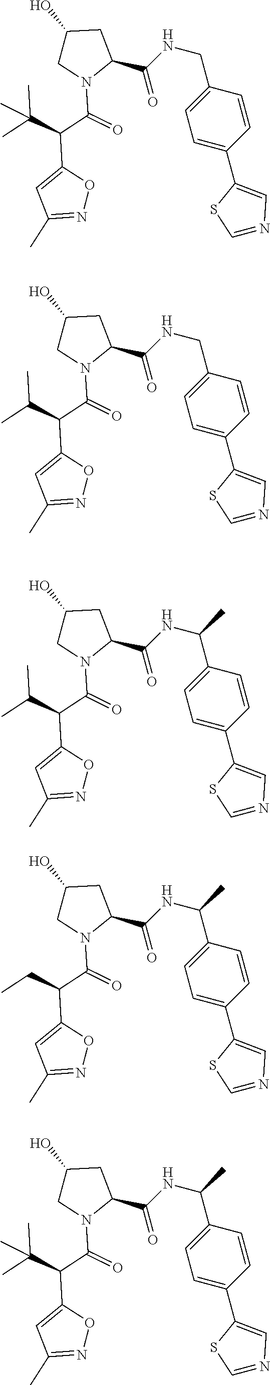

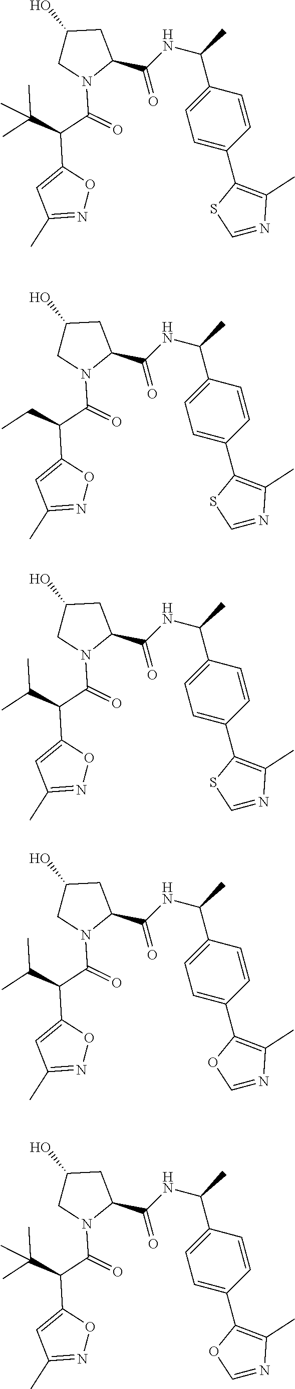

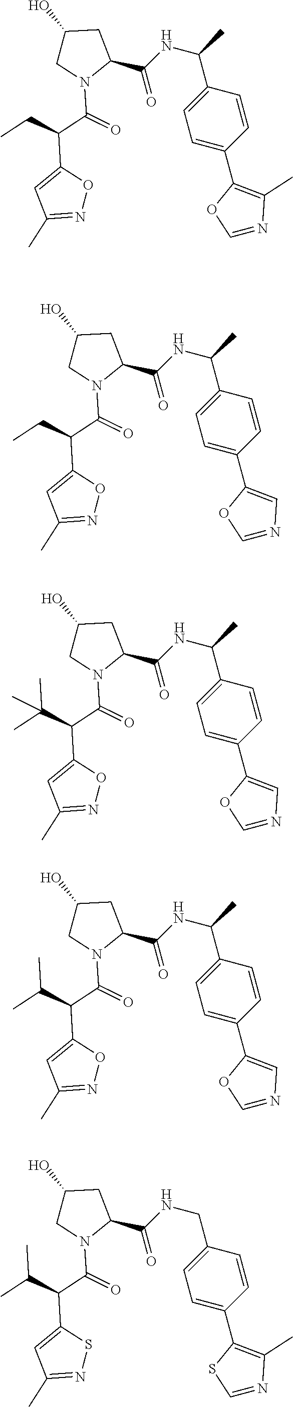

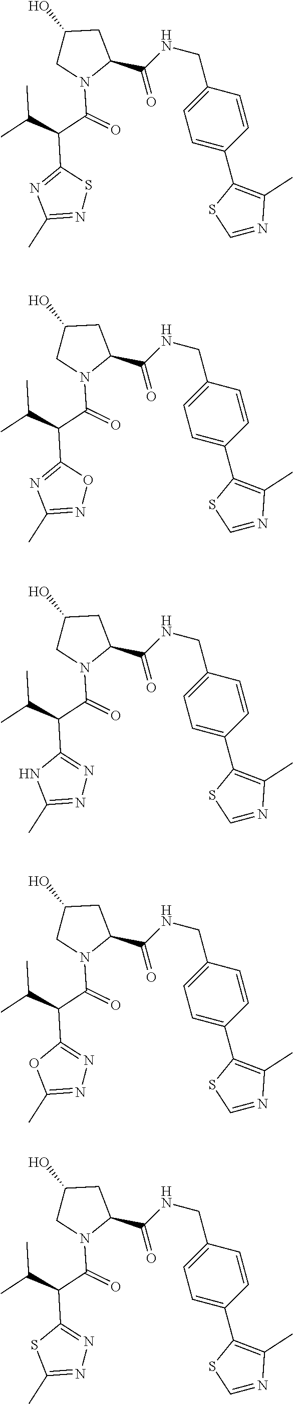

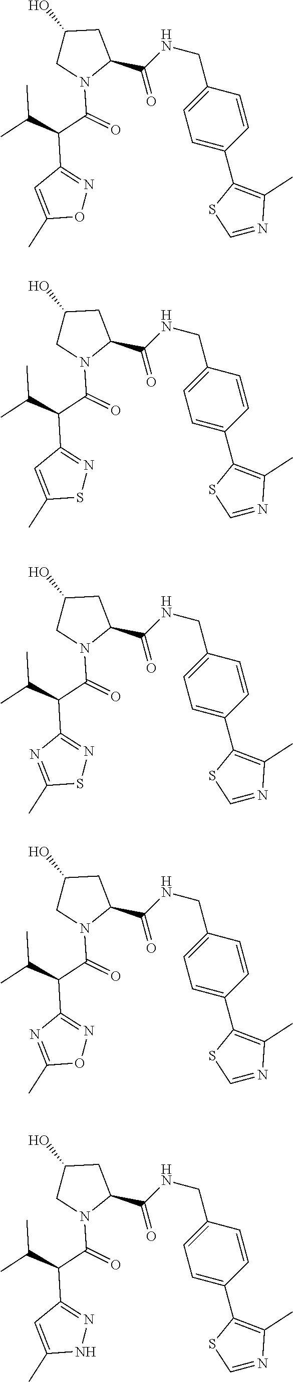

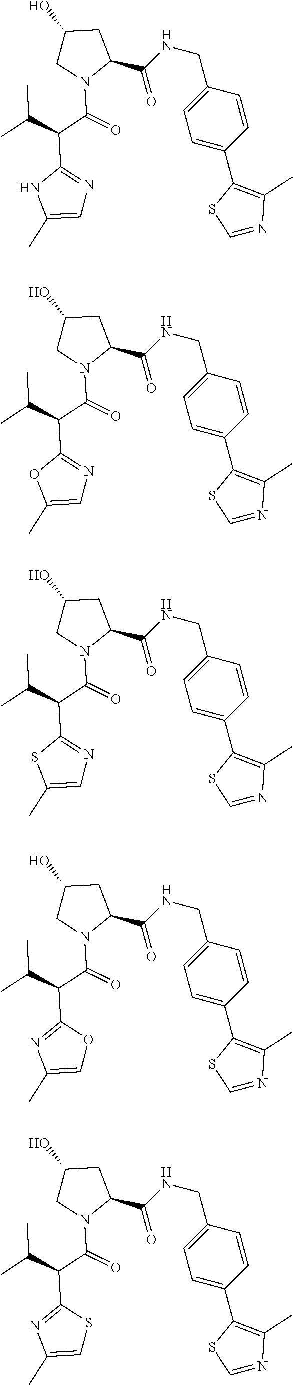

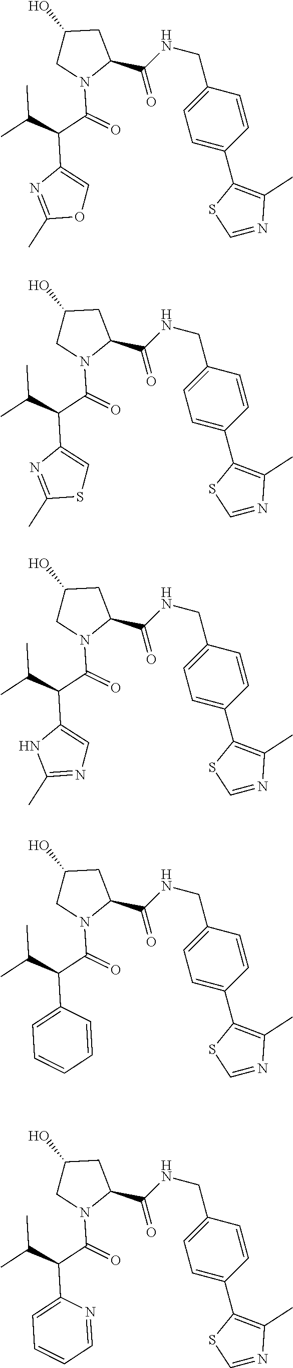

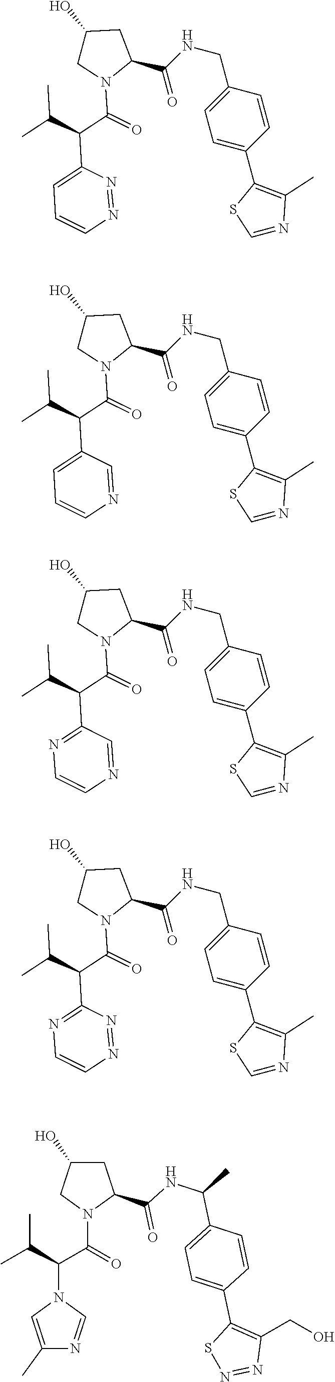

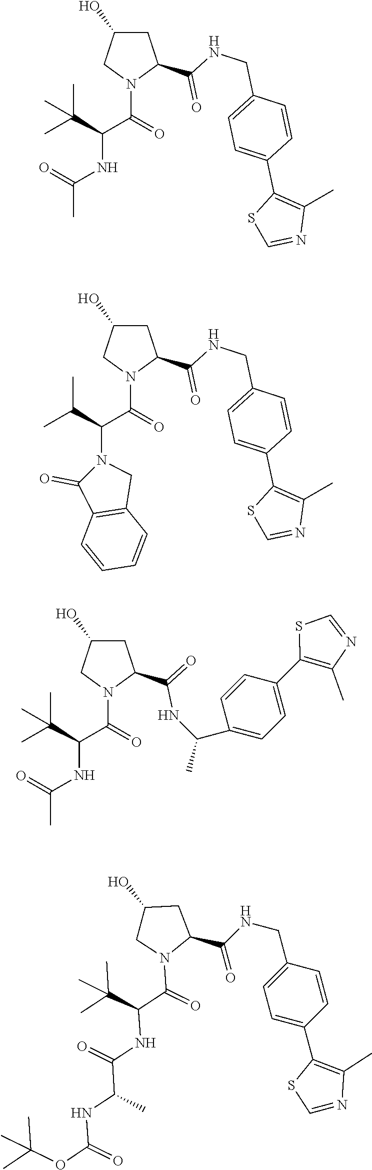

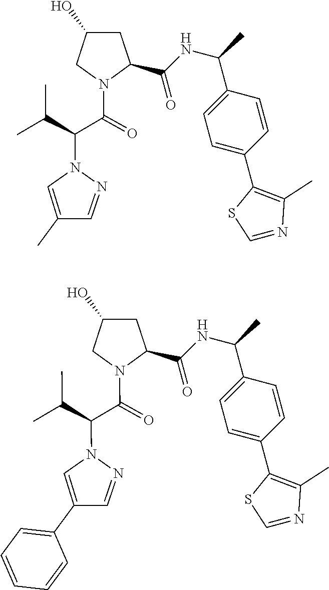 [SMILES: Cc1cnn([C@H](C(=O)N2C[C@H](O)C[C@H]2C(=O)N[C@@H](C)c2ccc(-c3scnc3C)cc2)C(C)C)c1 ; Cc1ncsc1-c1ccc([C@H](C)NC(=O)[C@@H]2C[C@@H](O)CN2C(=O)[C@H](C(C)C)n2cc(-c3ccccc3)cn2)cc1]

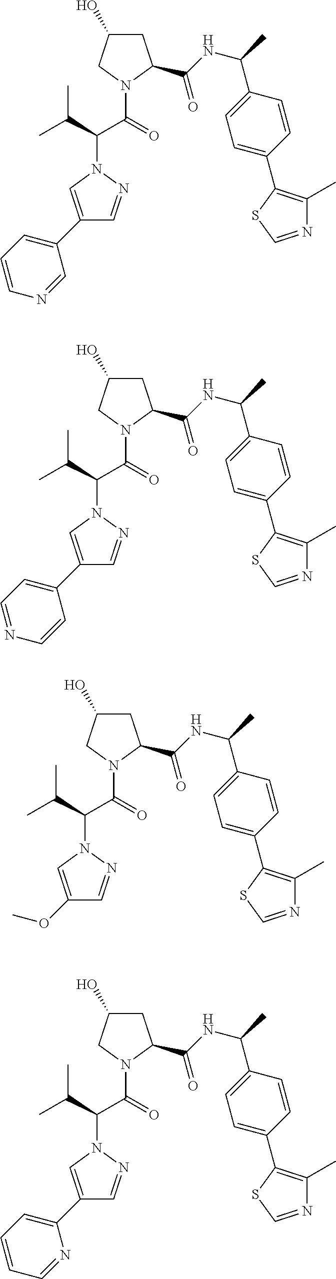

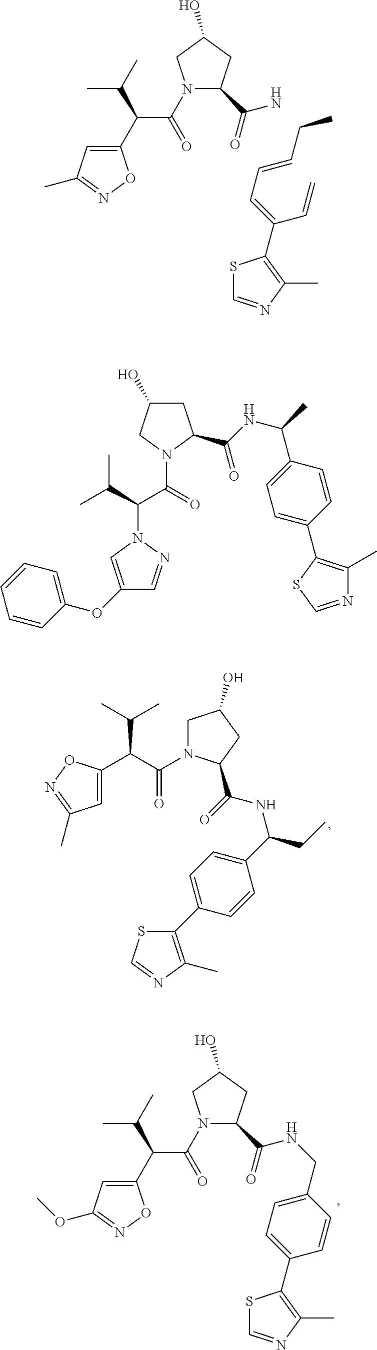

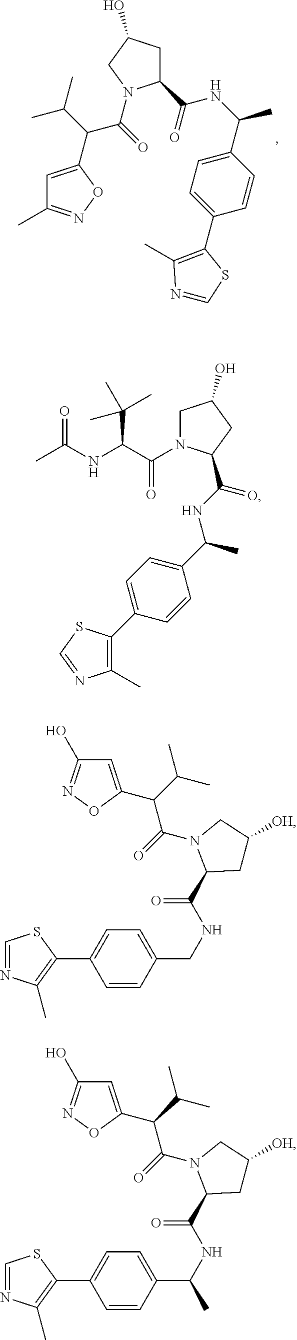

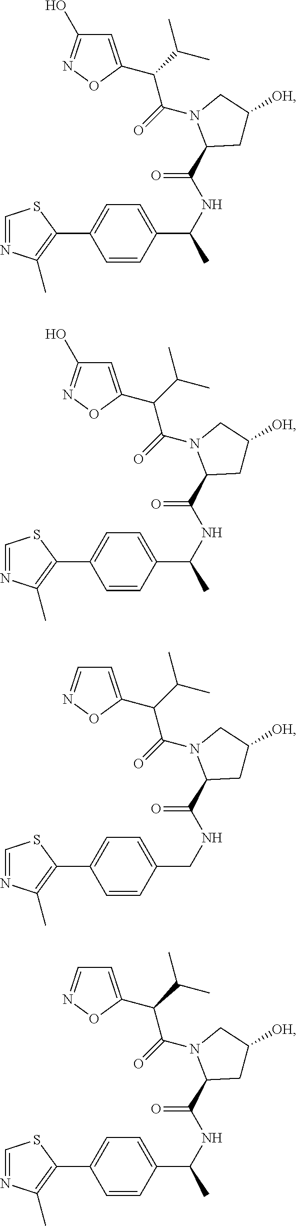

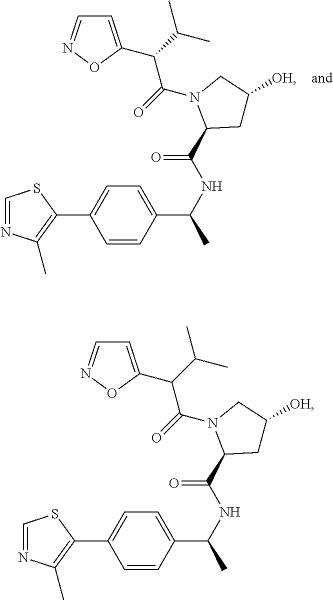 [SMILES: Cc1ncsc1-c1ccc([C@H](C)NC(=O)[C@@H]2C[C@@H](O)CN2C(=O)[C@H](c2ccno2)C(C)C)cc1 ; Cc1ncsc1-c1ccc([C@H](C)NC(=O)[C@@H]2C[C@@H](O)CN2C(=O)C(c2ccno2)C(C)C)cc1]

wherein the VLM may be connected to a PTM via a linker, as described herein, at any appropriate location, including, e.g., an aryl, heteroary, phenyl, or phenyl of an indole group, optionally via any appropriate functional group, such as an amine, ester, ether, alkyl, or alkoxy.

### Exemplary Linkers

In certain embodiments, the compounds as described herein include a means for chemically coupling the PTM to the ULM, e.g., one or more PTMs chemically linked or coupled to one or more ULMs (e.g., at least one of VLM) via a chemical linker (L). In certain embodiments, the linker group L is a group comprising one or more covalently connected structural units (e.g., -A^{L}₁ . . . (A^{L})_{q}- or -(A^{L})_{q}-), wherein A^{L}₁ is a group coupled to PTM, and (A^{L})_{q} is a group coupled to ULM.

In any aspect or embodiment described herein, the linker (L) to ULM (e.g., VLM, ILM, CLM, or MLM) connection or coupling is a stable L-ULM connection. For example, in any aspect or embodiment described herein, when a linker (L) and a ULM is connected via a heteroatom, any subsequent heteroatom, if present, is separated by at least one single carbon atom (e.g., —CH₂—), such as with an acetal or aminal group. By way of further example, in any aspect or embodiment described herein, when a linker (L) and a ULM is connected via a heteroatom, the heteroatom is not part of a ester.

In any aspect or embodiment described herein, the linker group L is a bond or a chemical linker group represented by the formula -(A^{L})_{q}-, wherein A is a chemical moiety and q is an integer from 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80), and wherein L is covalently bound to both the PTM and the ULM, and provides for binding of the PTM to the protein target and the ULM to an E3 ubiquitin ligase to effectuate target protein ubiquitination.

In any aspect or embodiment described herein, the linker group L is a bond or a chemical linker group represented by the formula -(A^{L})_{q}-, wherein A is a chemical moiety and q is an integer from 6-30 (e.g., 1, 2, 3, 4, 5, 6, 7, 8, 9, 10, 11, 12, 13, 14, 15, 16, 17, 18, 19, 20, 21, 22, 23, 24, or 25), and wherein L is covalently bound to both the PTM and the ULM, and provides for binding of the PTM to the protein target and the ULM to an E3 ubiquitin ligase in sufficient proximity to result in target protein ubiquitination.

In any aspect or embodiment described herein, the linker group L is -(A^{L})_{q}-, wherein:
(A^{L})_{q} is a group which is connected to at least one of a ULM (such as a VLM), PTM moiety, or a combination thereof;
q of the linker is an integer greater than or equal to 1;
each A^{L} is independently selected from the group consisting of, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₅₋₁₃ spirocycloalkyl optionally substituted with 0-9 R^{L1} and/or R^{L2} groups, C₃₋₁₁heterocyclyl optionally substituted with 0-6 R^{L1} and/or R^{L2} groups, C₅₋₁₃ spiroheterocyclyl optionally substituted with 0-8 R^{L}₁ and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and
R^{L1}, R^{L2}, R^{L3}, R^{L} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NHSO₂N(C₁₋₈alkyl)₂, NH SO₂NH₂.

In any aspect or embodiment described herein, each A^{L} is independently selected from the group consisting of CR^{L1}R^{L2}, O, NR^{L3}, CONR^{L3}, NR^{L3}CONR^{L4}, CO, CR^{L1}=CR^{L2}, C≡C, C₃₋₁₁cycloalkyl optionally substituted with 1-6 R^{L1} and/or R^{L2} groups, C₃₋₁₁heterocyclyl optionally substituted with 1-6 R^{L1} and/or R^{L2} groups, aryl optionally substituted with 1-6 R^{L1} and/or R^{L2} groups, and heteroaryl optionally substituted with 1-6 R^{L1} and/or R^{L2} groups, where R^{L1} or R^{L2}, each independently are optionally linked to other groups to form cycloalkyl and/or heterocyclyl moiety, optionally substituted with 1-4 R^{L5} groups; and R^{L1}, R^{L2}, R^{L3}, and R^{L} are, each independently, halogen, C₁₋₈alkyl, OC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₃₋₈cycloalkyl, NHC₃₋₈cycloalkyl, N(C₃₋₈cycloalkyl)₂, N(C₃₋₈cycloalkyl)(C₁₋₈alkyl), OH, NH₂, CCH, COC₁₋₈alkyl, CO₂H, halogen, CN, CF₃, CHF₂, CH₂F, or NO₂.

In certain embodiments, q of the linker is an integer greater than or equal to 0. In certain embodiments, q is an integer greater than or equal to 1.

In certain embodiments, e.g., where q of the linker is greater than 2, (A^{L})_{q} is a group which is to A^{L}₁ and (A^{L})_{q} wherein the units A^{L} couple a PTM to a ULM.

In certain embodiments, e.g., where q of the linker is 2, (A^{L})_{q} is a group which is connected to A^{L}₁ and to a ULM or PTM.

In certain embodiments, e.g., where q of the linker is 1, the structure of the linker group L is -A^{L}₁-, and A^{L}₁ is a group which is connected to a ULM moiety and a PTM moiety.

In certain embodiments, the unit A^{L} of linker (L) comprises a group represented by a general structure selected from the group consisting of:
—NR(CH₂)ₙ-(lower alkyl)-, —NR(CH₂)ₙ-(lower alkoxyl)-, —NR(CH₂)ₙ-(lower alkoxyl)-OCH₂—, —NR(CH₂)ₙ-(lower alkoxyl)-(lower alkyl)-OCH₂—, —NR(CH₂)ₙ-(cycloalkyl)-(lower alkyl)-OCH₂—, —NR(CH₂)ₙ-(hetero cycloalkyl)-, —NR(CH₂CH₂O)ₙ-(lower alkyl)-O—CH₂—, —NR(CH₂CH₂O)ₙ-(hetero cycloalkyl)-O—CH₂—, —NR(CH₂CH₂O)ₙ-Aryl-O—CH₂—, —NR(CH₂CH₂O)ₙ-(hetero aryl)-O—CH₂—, —NR(CH₂CH₂O)ₙ-(cyclo alkyl)-O-(hetero aryl)-O—CH₂—, —NR(CH₂CH₂O)ₙ-(cyclo alkyl)-O-Aryl-O—CH₂—, —NR(CH₂CH₂O)ₙ-(lower alkyl)-NH-Aryl-O—CH₂—, —NR(CH₂CH₂O)ₙ-(lower alkyl)-O-Aryl-CH₂, —NR(CH₂CH₂O)ₙ-cycloalkyl-O-Aryl-, —NR(CH₂CH₂O)ₙ-cycloalkyl-O-(heteroaryl)l-, —NR(CH₂CH₂)ₙ-(cycloalkyl)-O-(heterocyclyl)-CH₂, —NR(CH₂CH₂)ₙ-(heterocyclyl)-(heterocyclyl)-CH₂, —N(R1R2)-(heterocyclyl)-CH₂; where
n of the linker can be 0 to 10;
R of the linker can be H, lower alkyl;
R1 and R2 of the linker can form a ring with the connecting N.

In any aspect or embodiment described herein, the L is selected from the group consisting of:

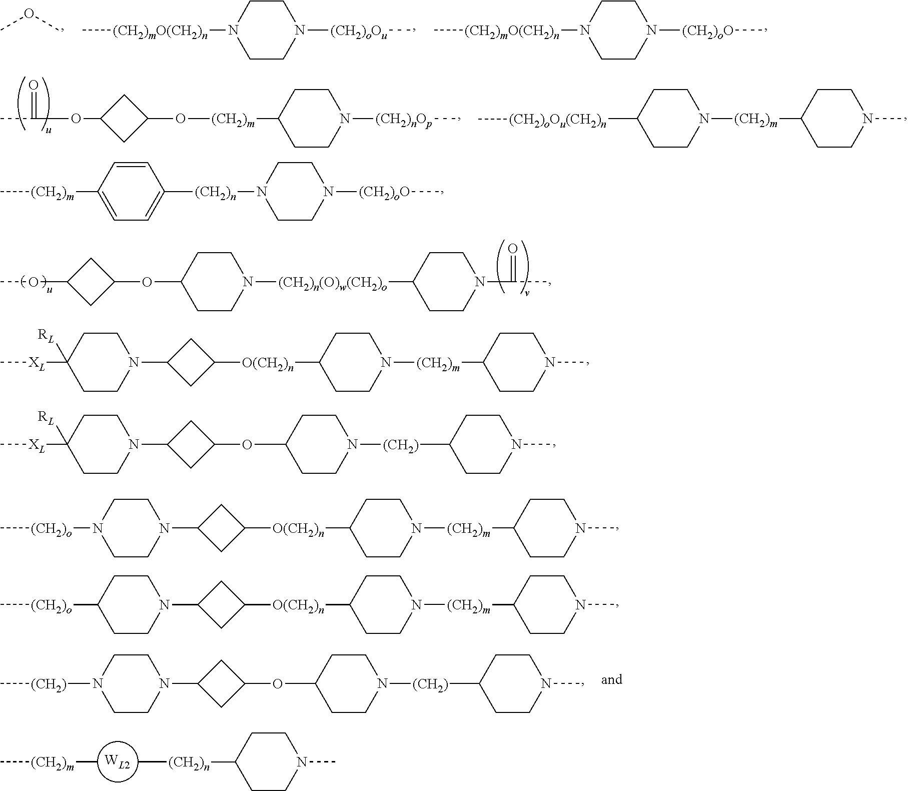

wherein:
each m, n, o, and p of the chemical linking moiety is independently selected from the integers 0, 1, 2, 3 and 4 (preferably 0, 1, or 2); and
each u, w, and v of the chemical liking moiety is independently selected from integers 0 and 1.
X_{L} is —C(CH₂)—, —C(CH₃)H,— —CH₂—, —O—, C═O, or —NH—CH₂—;
R_{L} is H, OH, F, Cl, or methyl; and
W_{L2} is selected from an optionally substituted 6-12 membered spirocycloalkyl or spiroheterocyclyl (e.g. a 6-12 or 8-12 member spirocycloalkyl or spiroheterocyclyl substituted with 0, 1, or 2 substituents selected from hydroxy, halogen, C₁₋₃ alkoxy, C₁₋₃ alkyl, C₁₋₃ haloalkyl, or amino), and

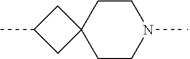

In any aspect or embodiment described herein, each m, n, o, and p of the chemical linking moiety is independently selected from the integers 0, 1, or 2.

In any aspect or embodiment described herein, the L is selected from the group consisting of:

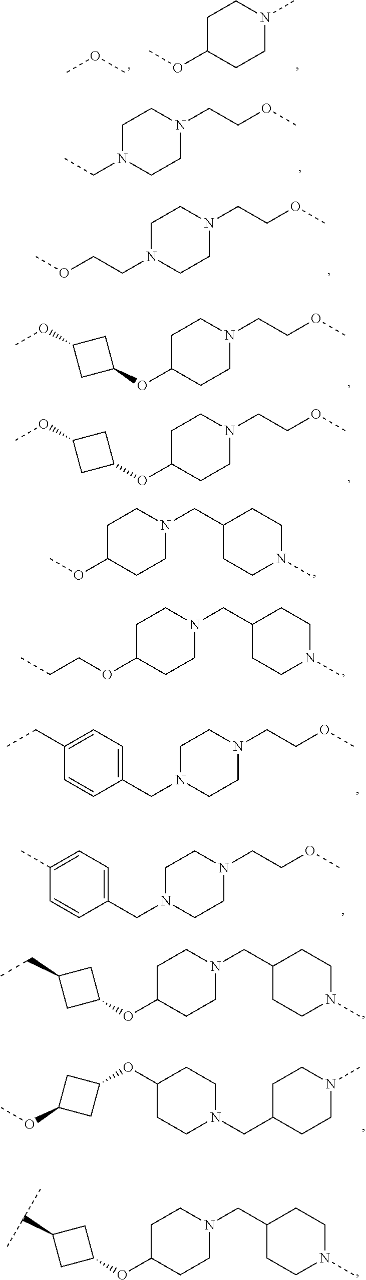

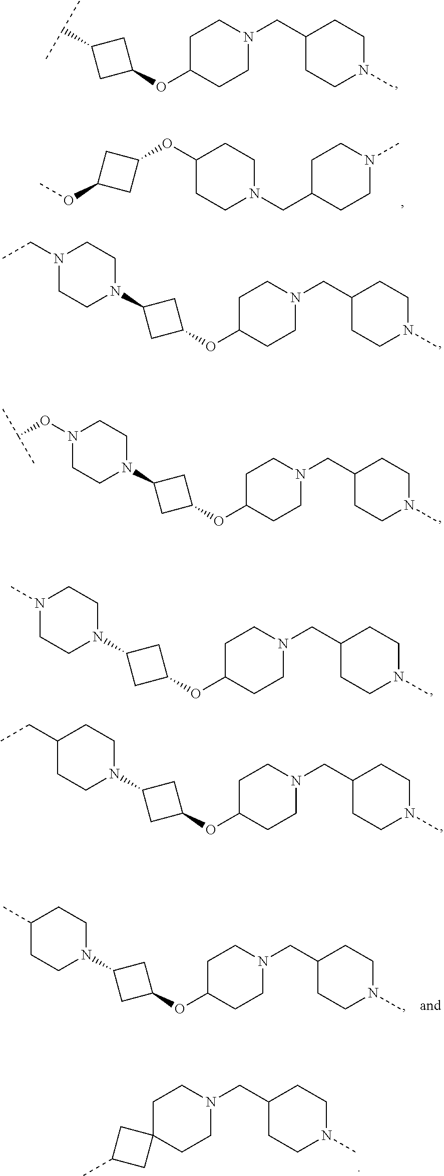

In any aspect or embodiment described herein, the unit A^{L} of linker (L) is selected from the group consisting of:

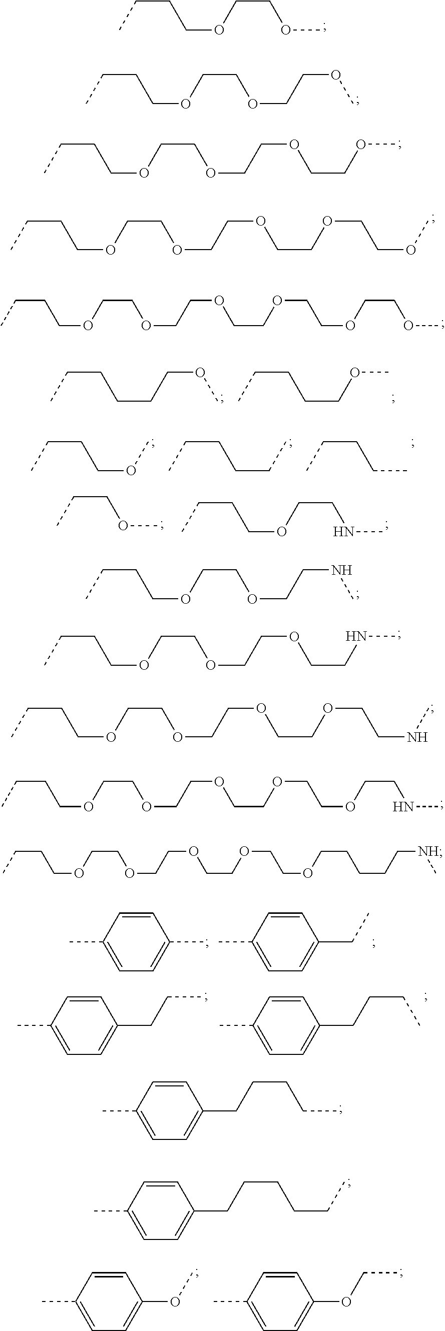

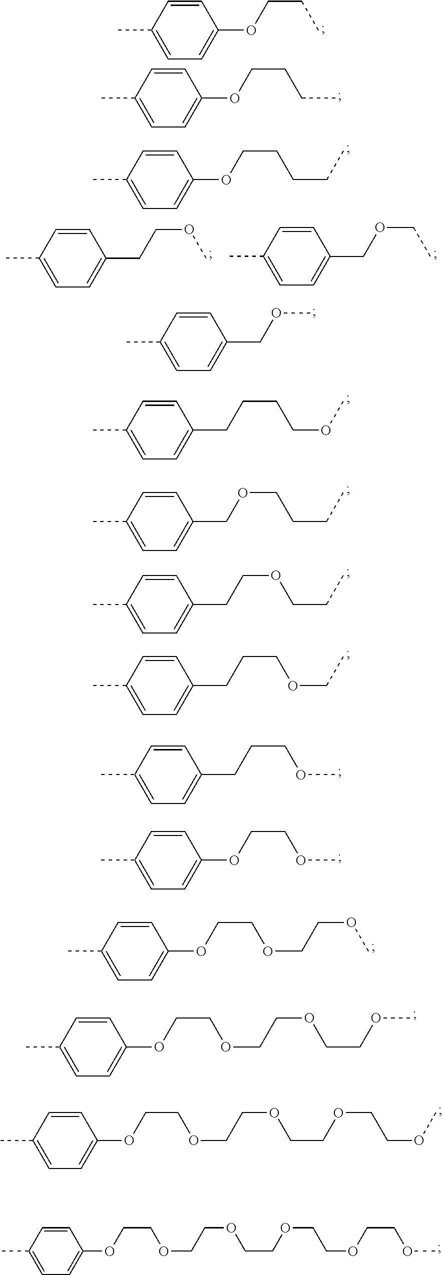

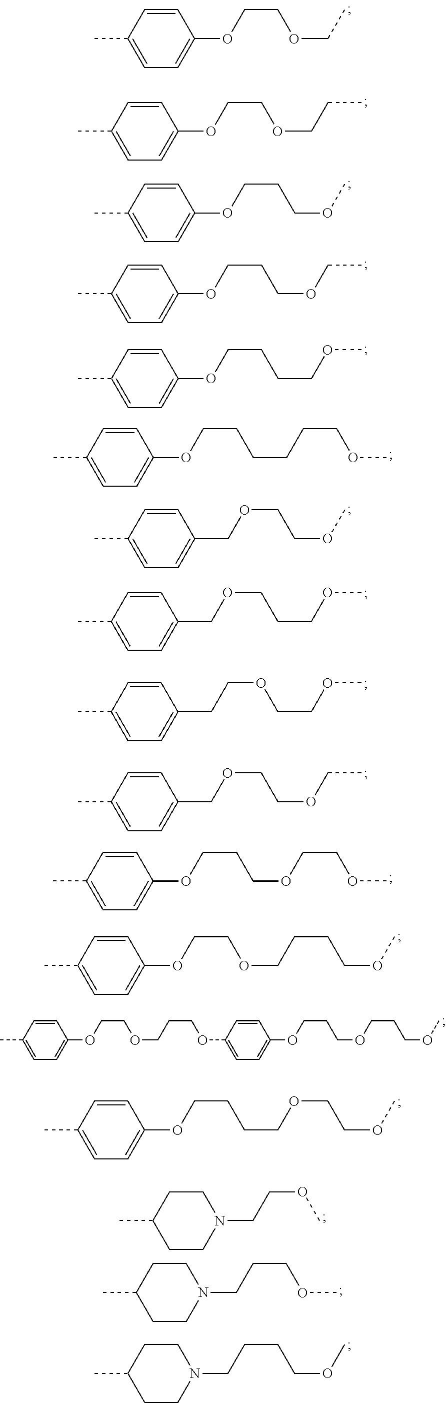

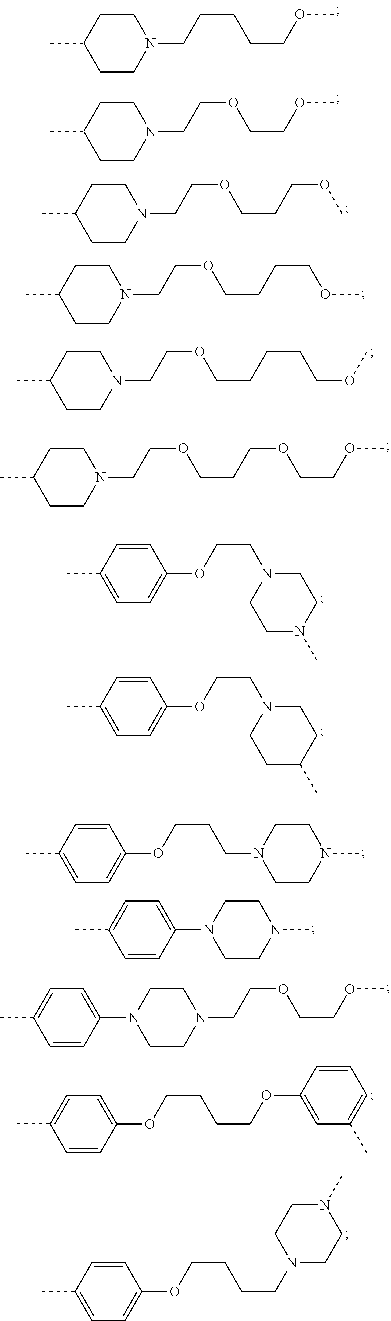

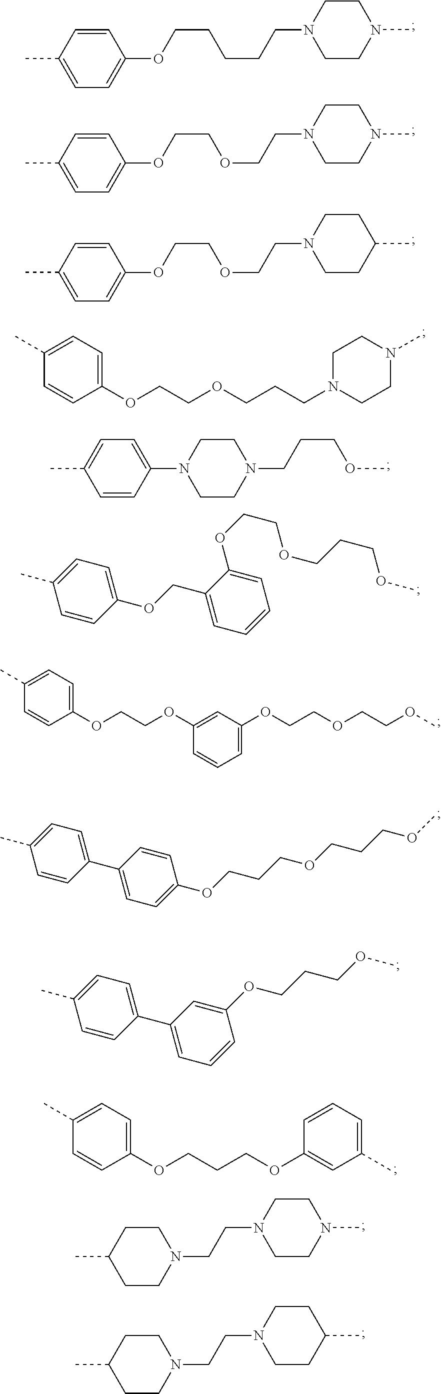

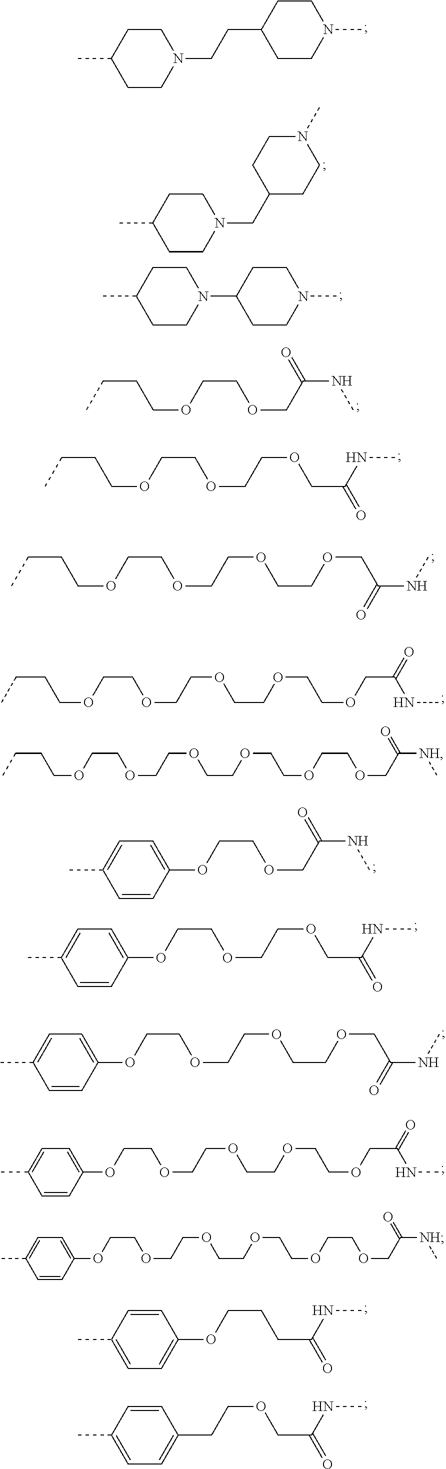

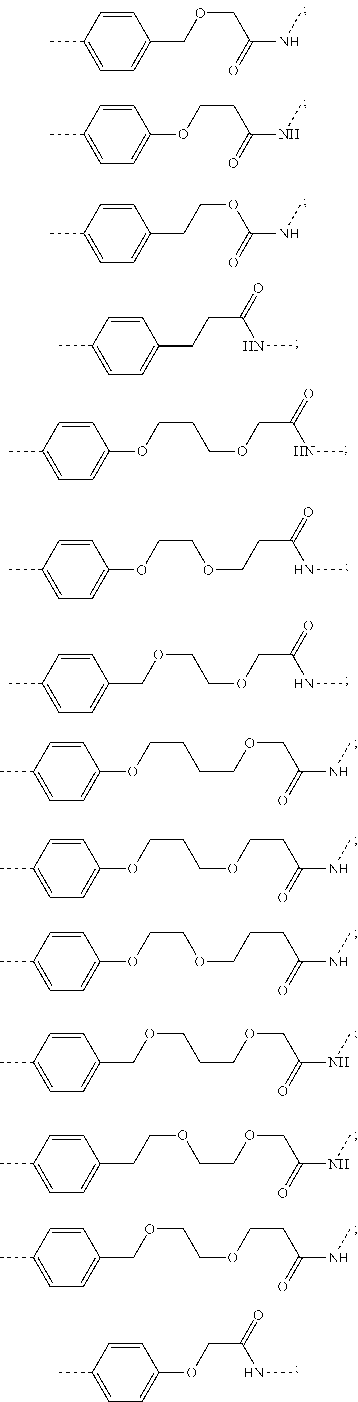

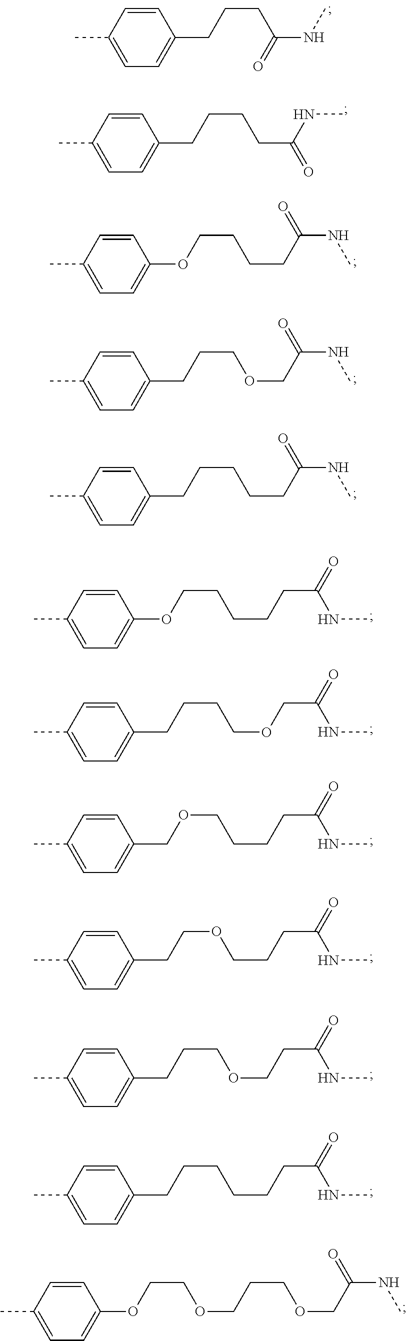

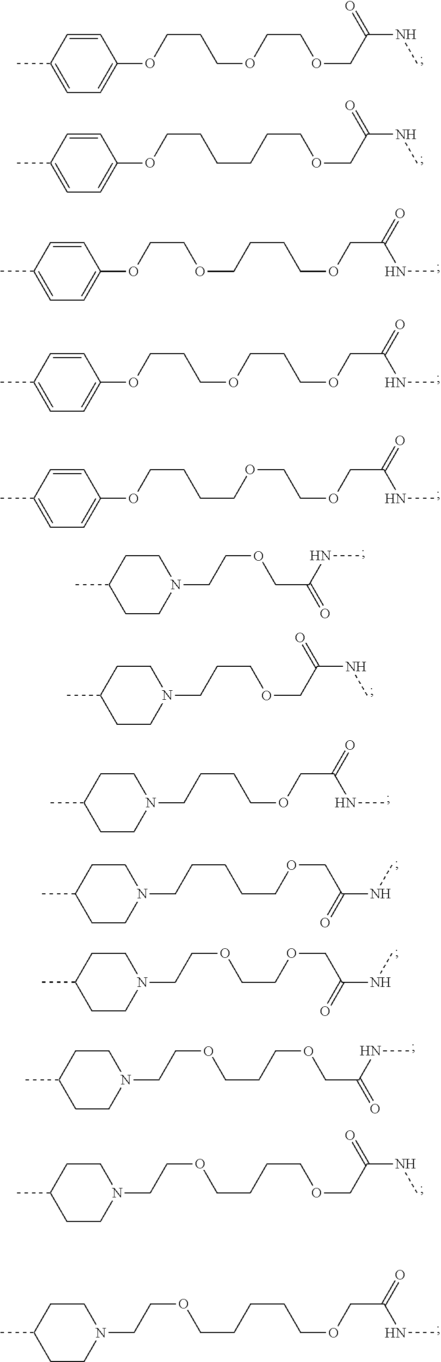

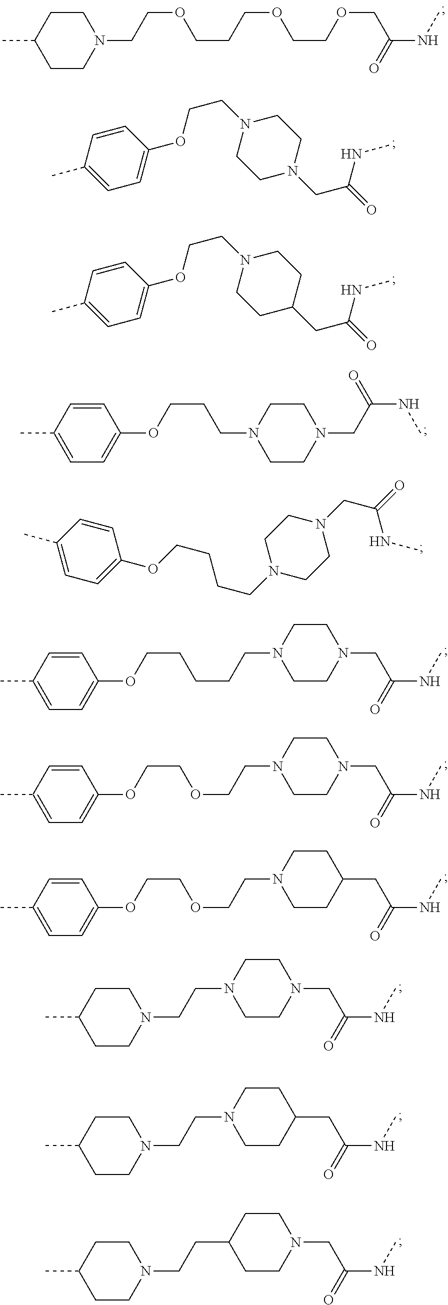

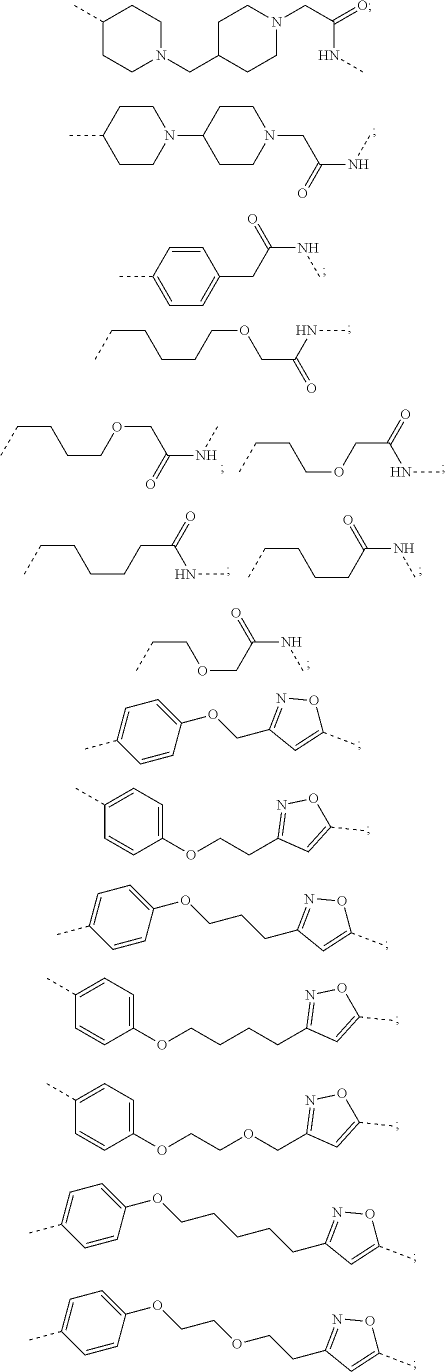

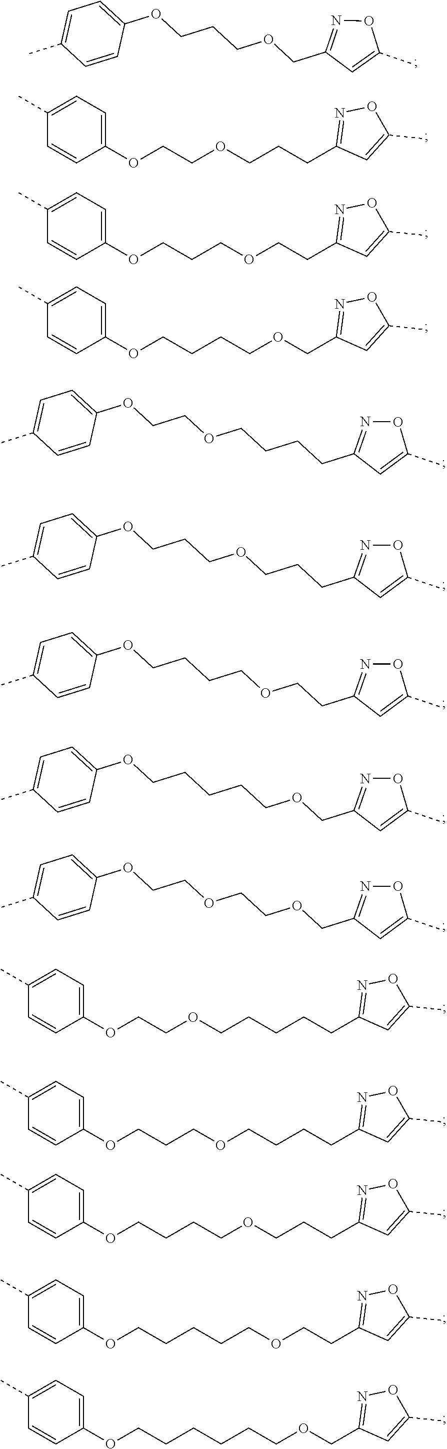

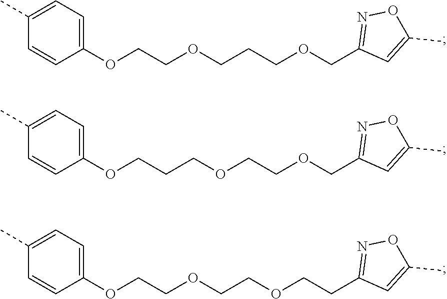

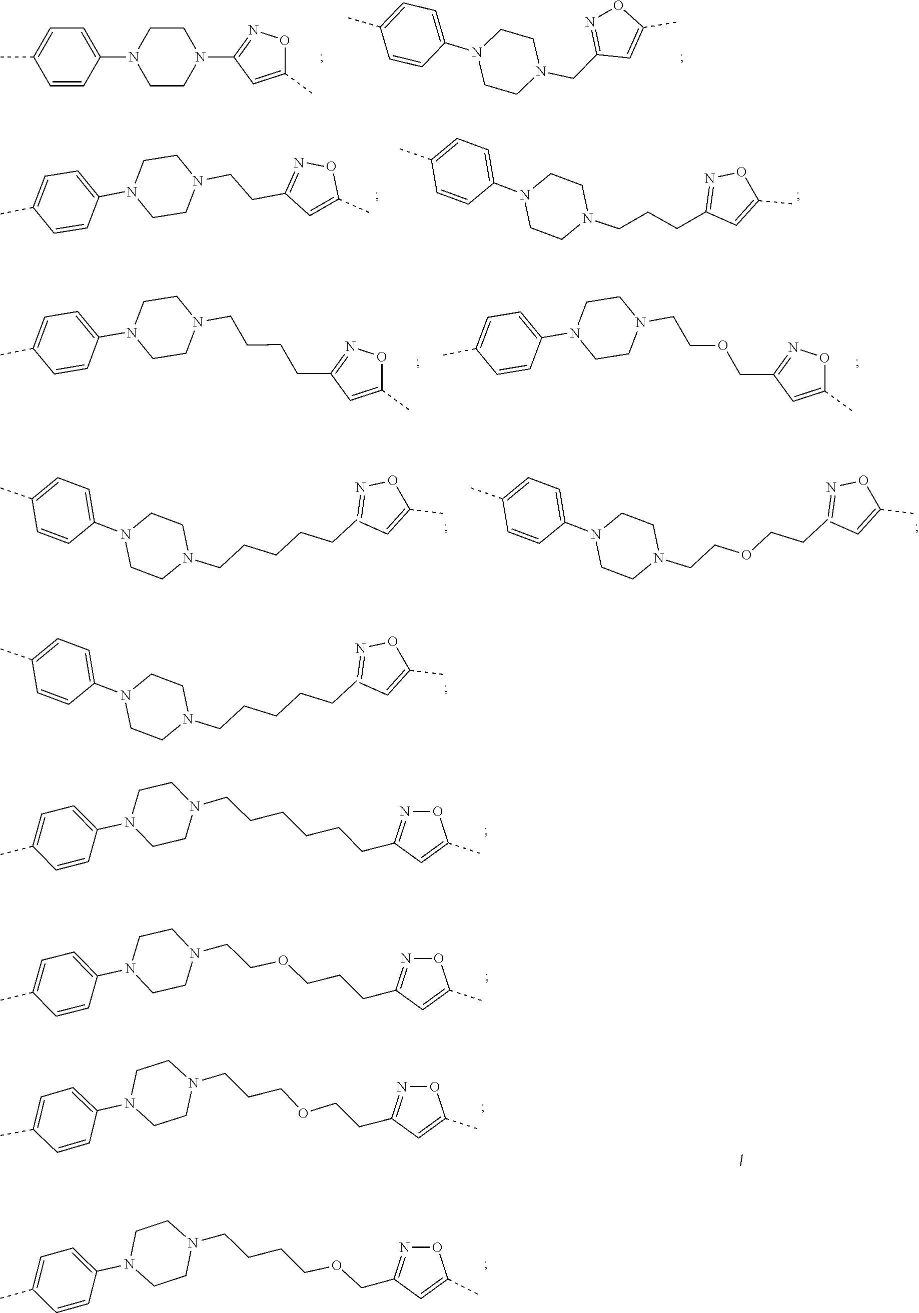

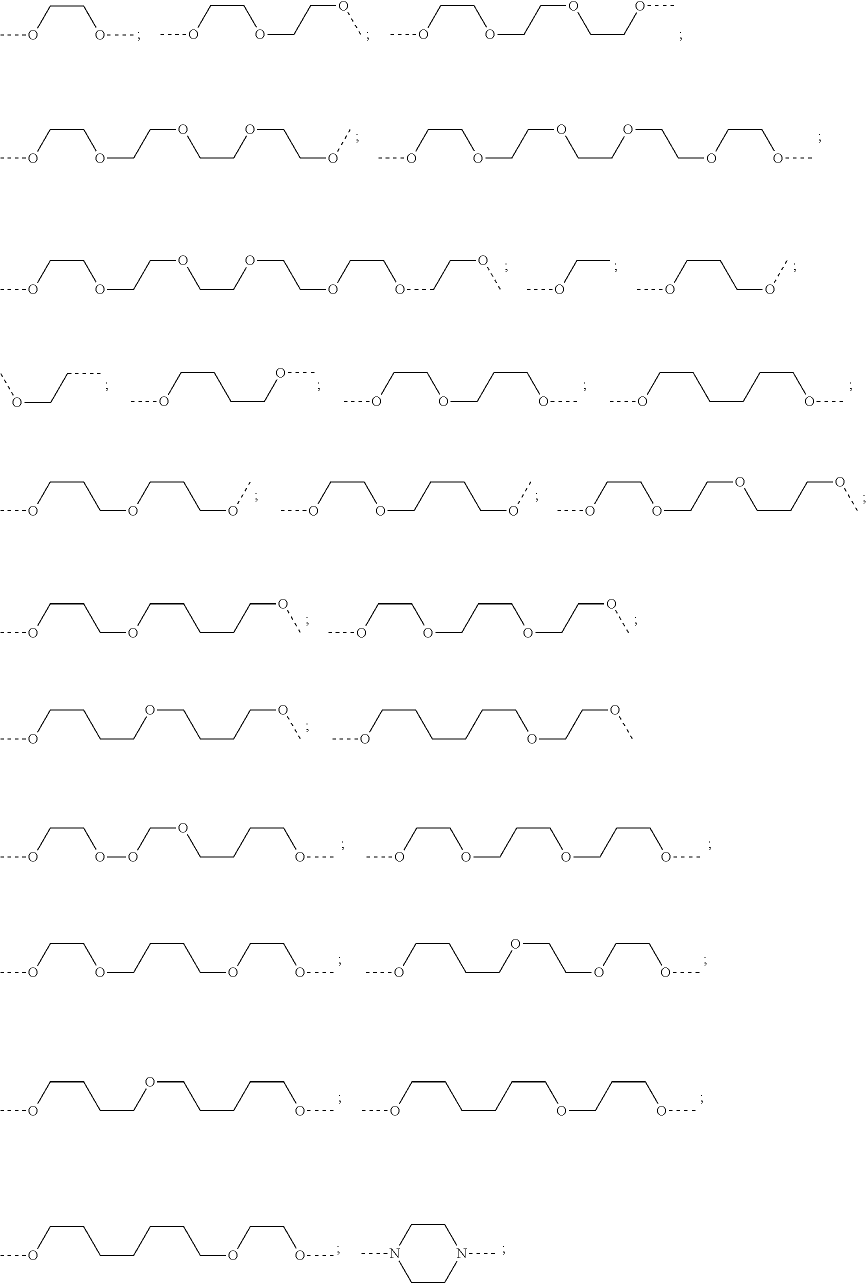

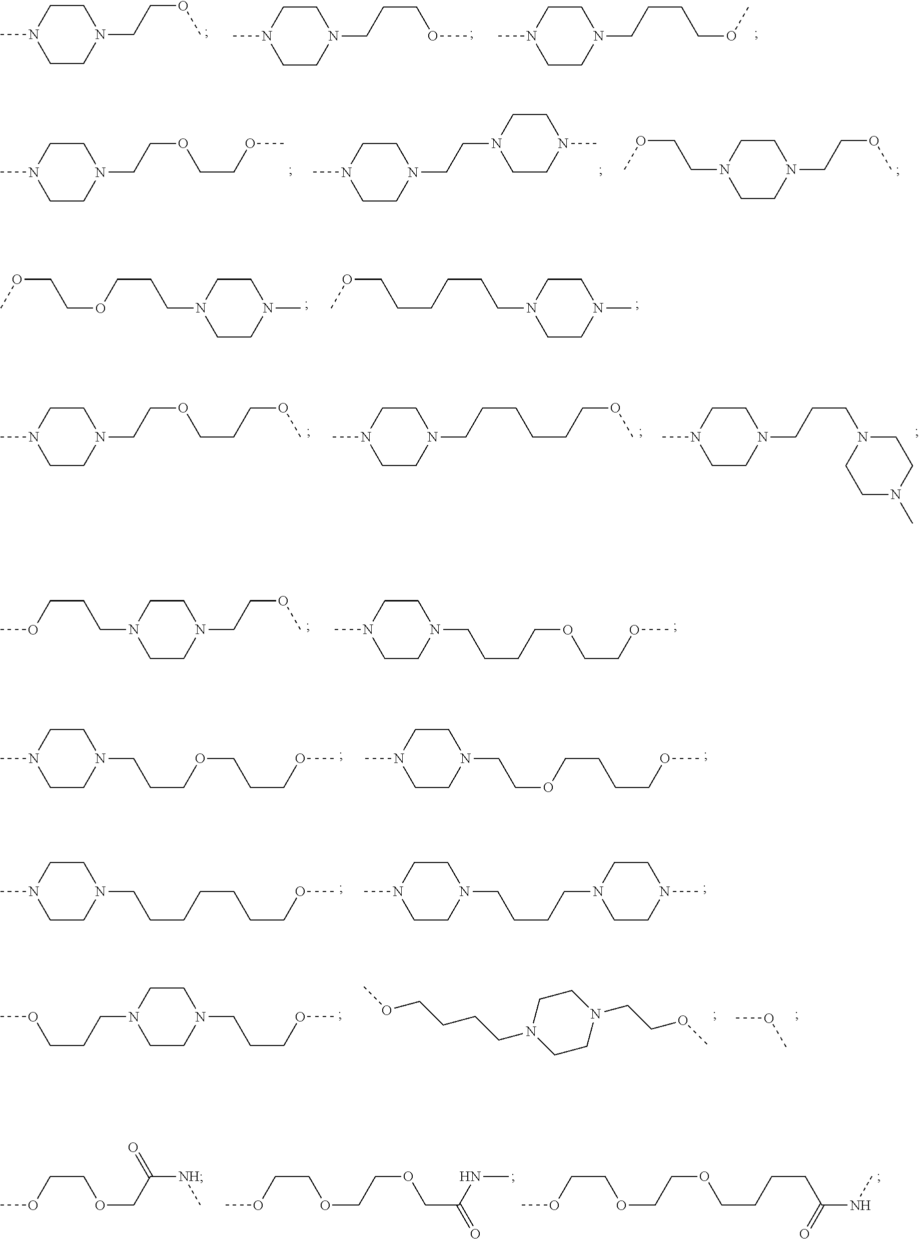

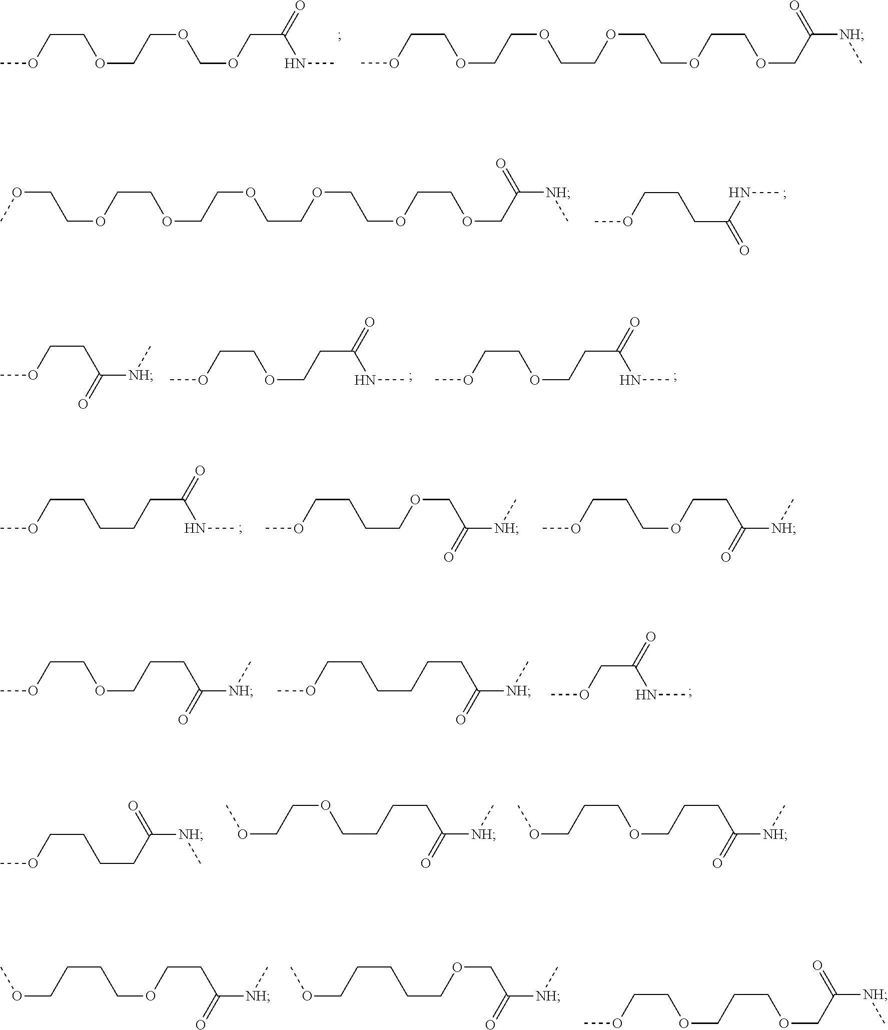

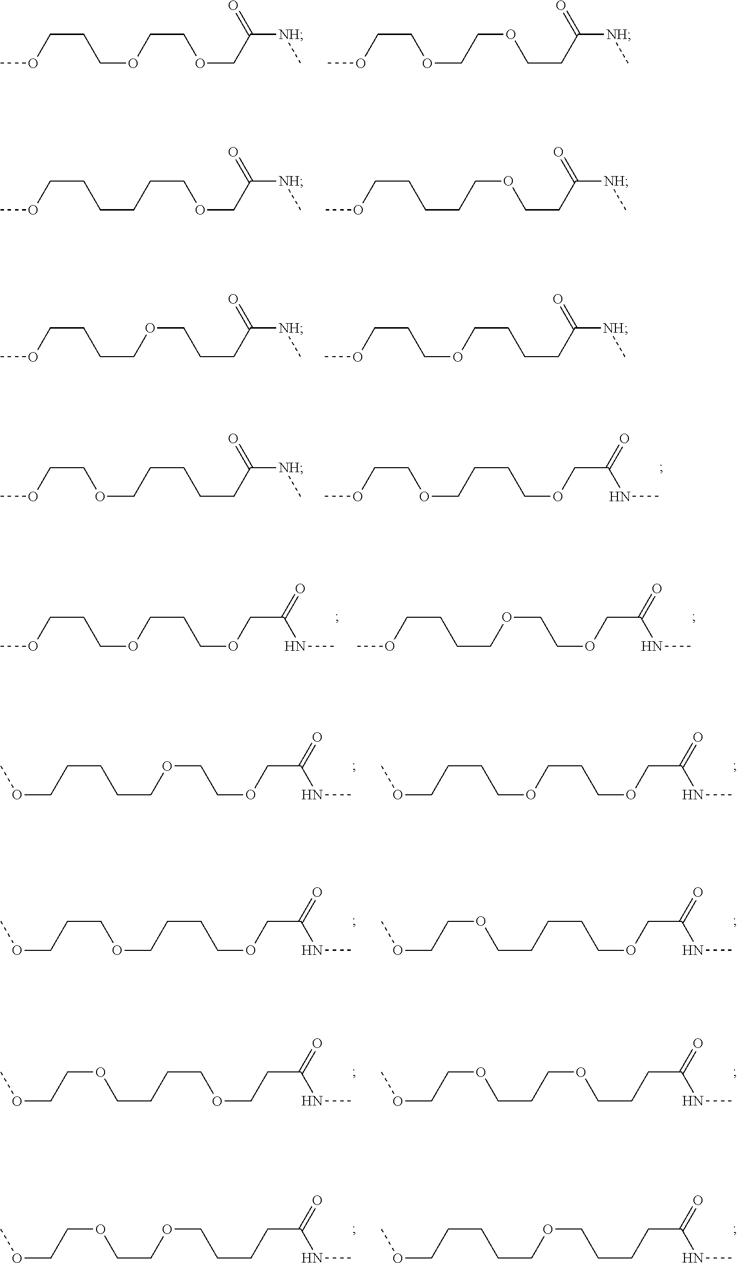

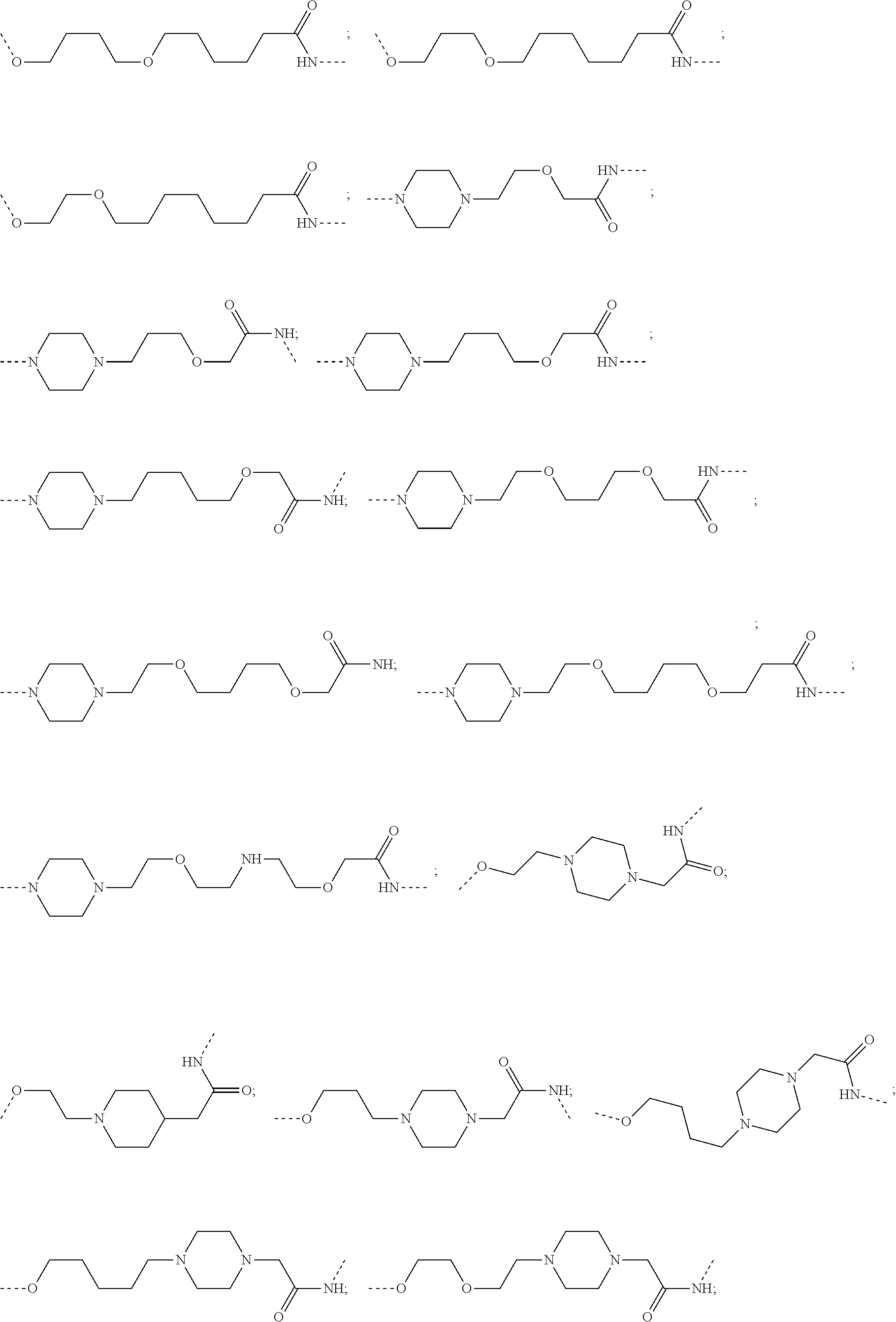

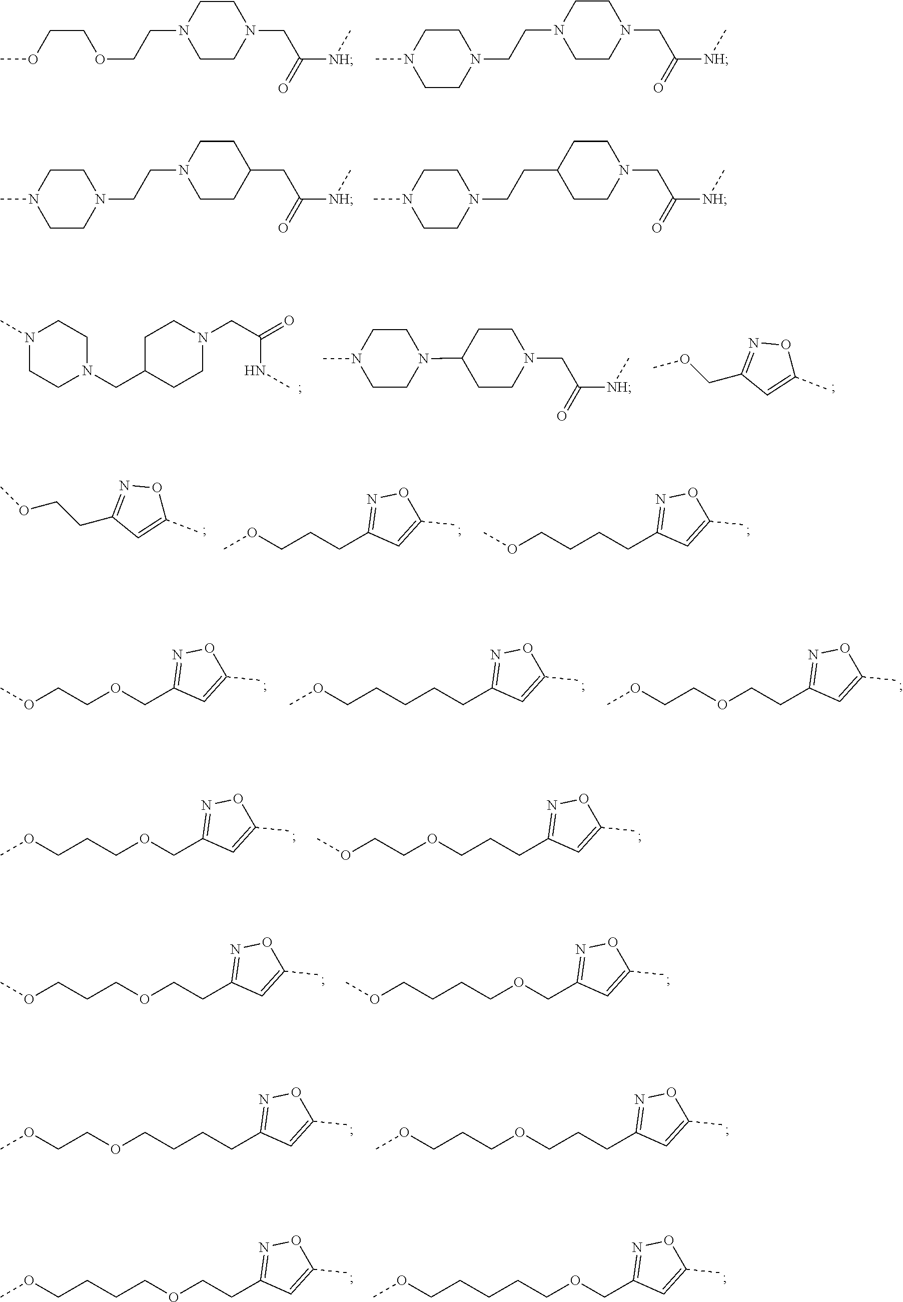

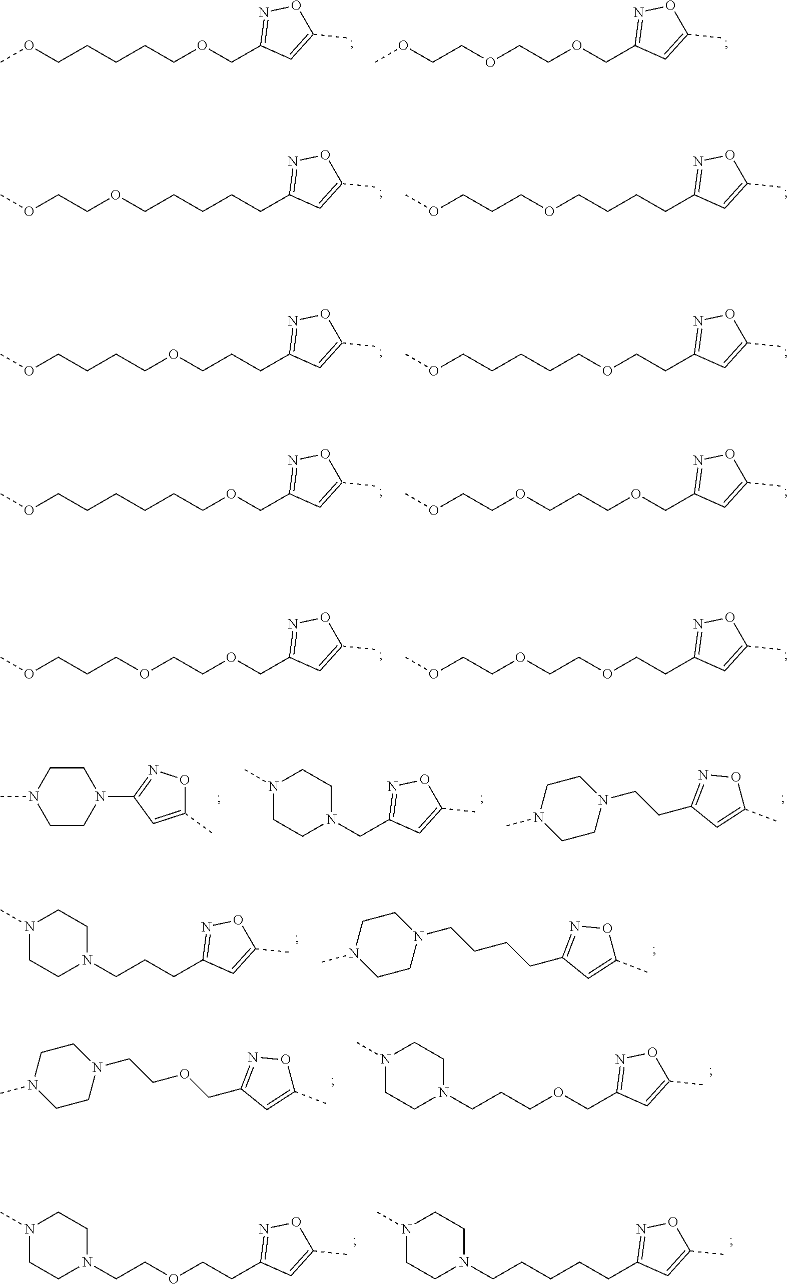

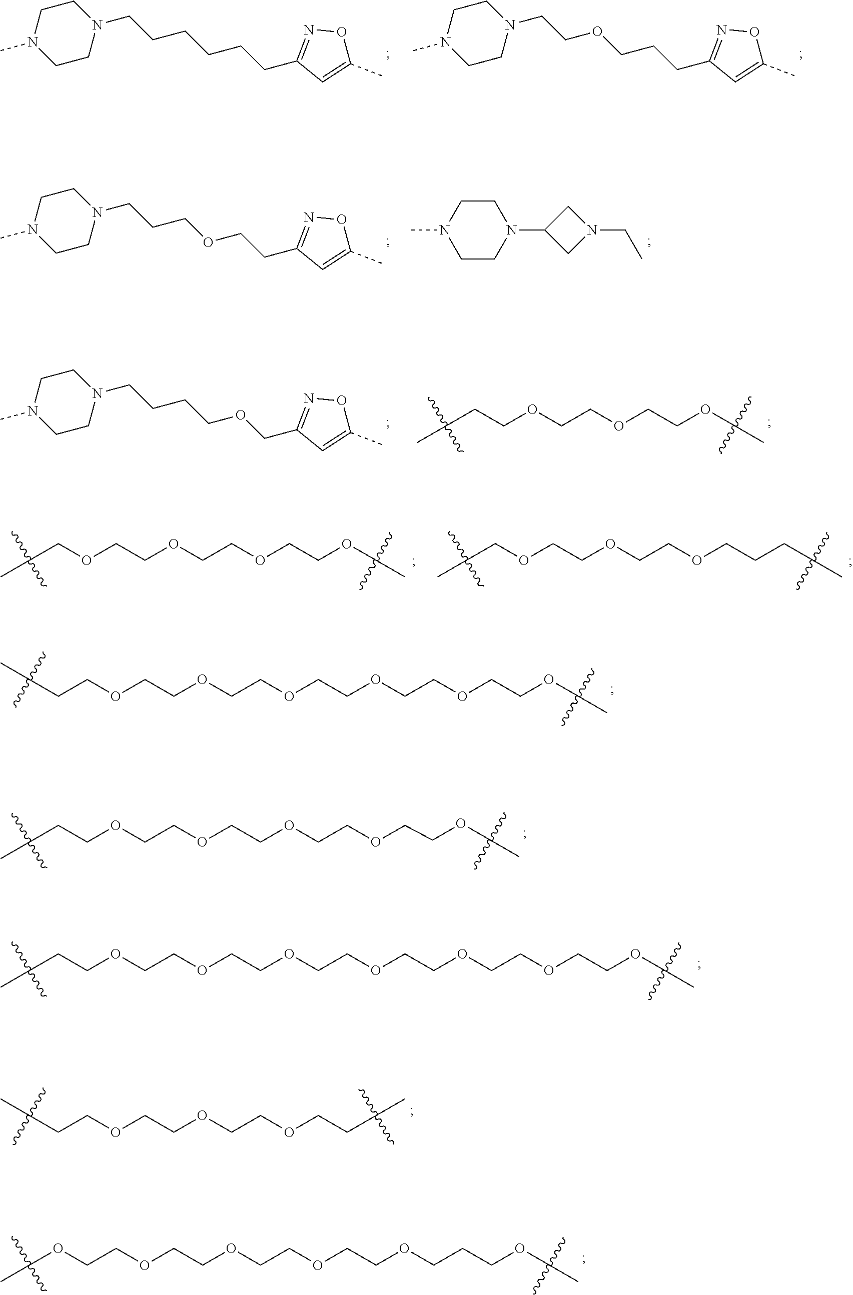

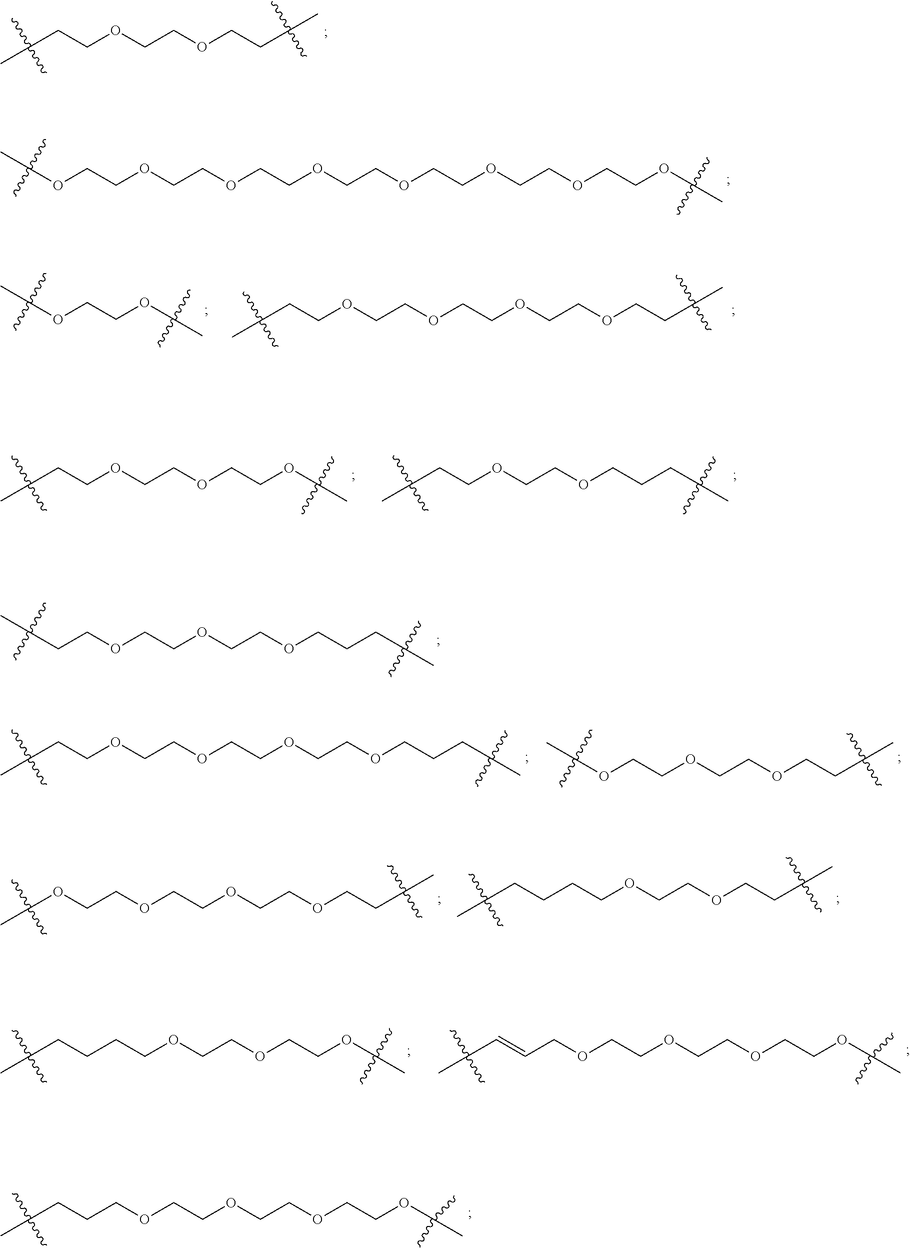

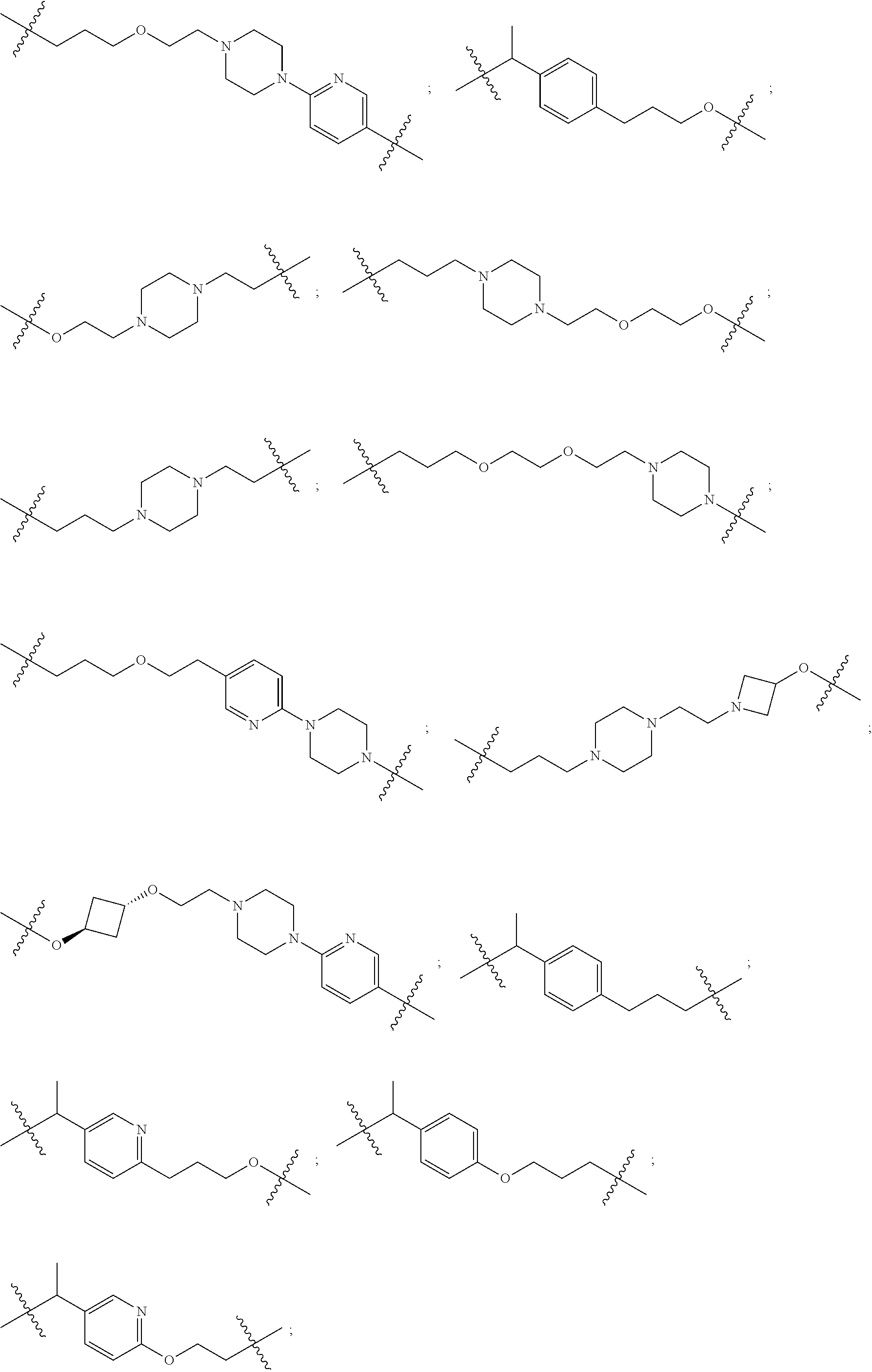

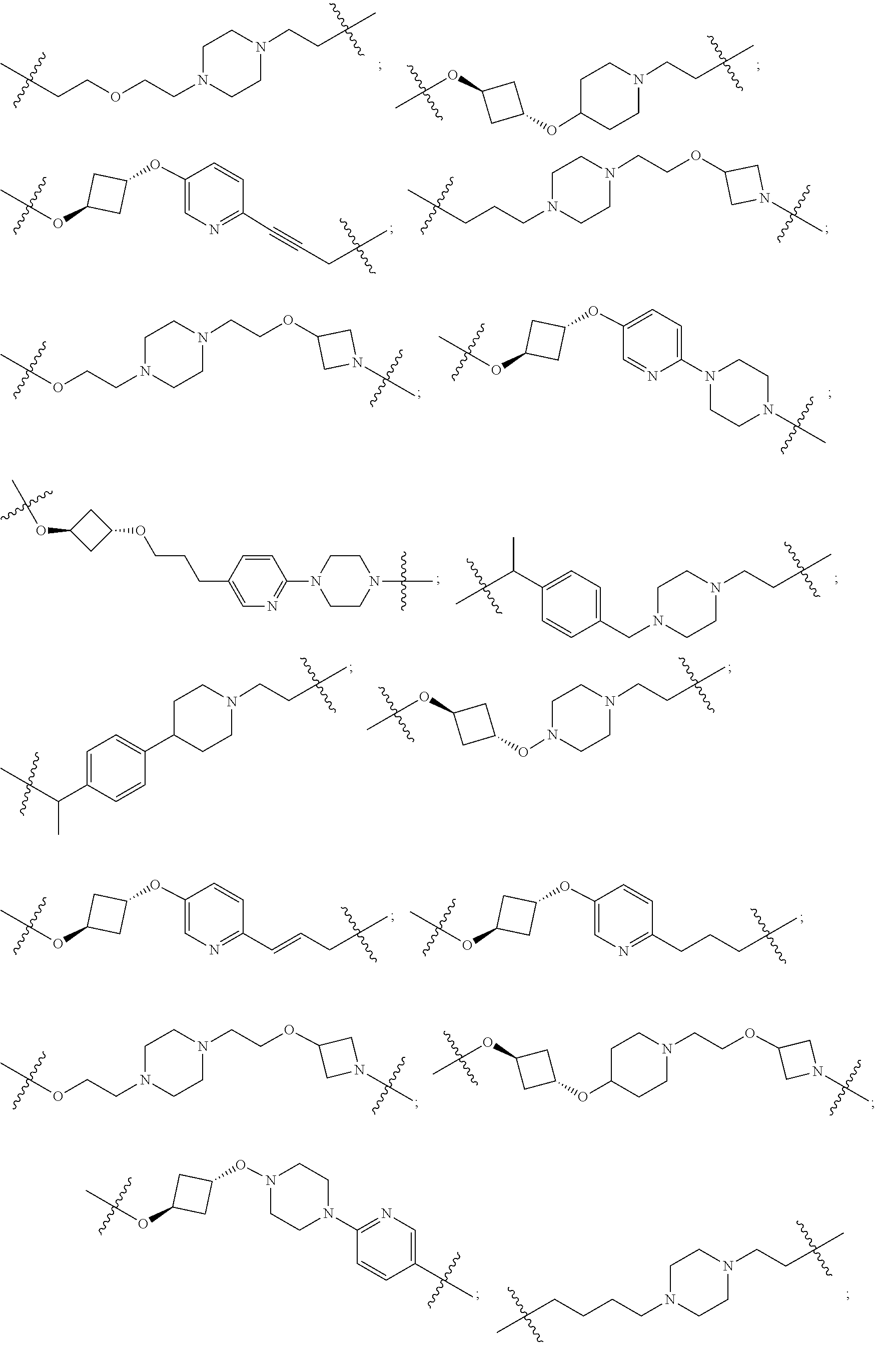

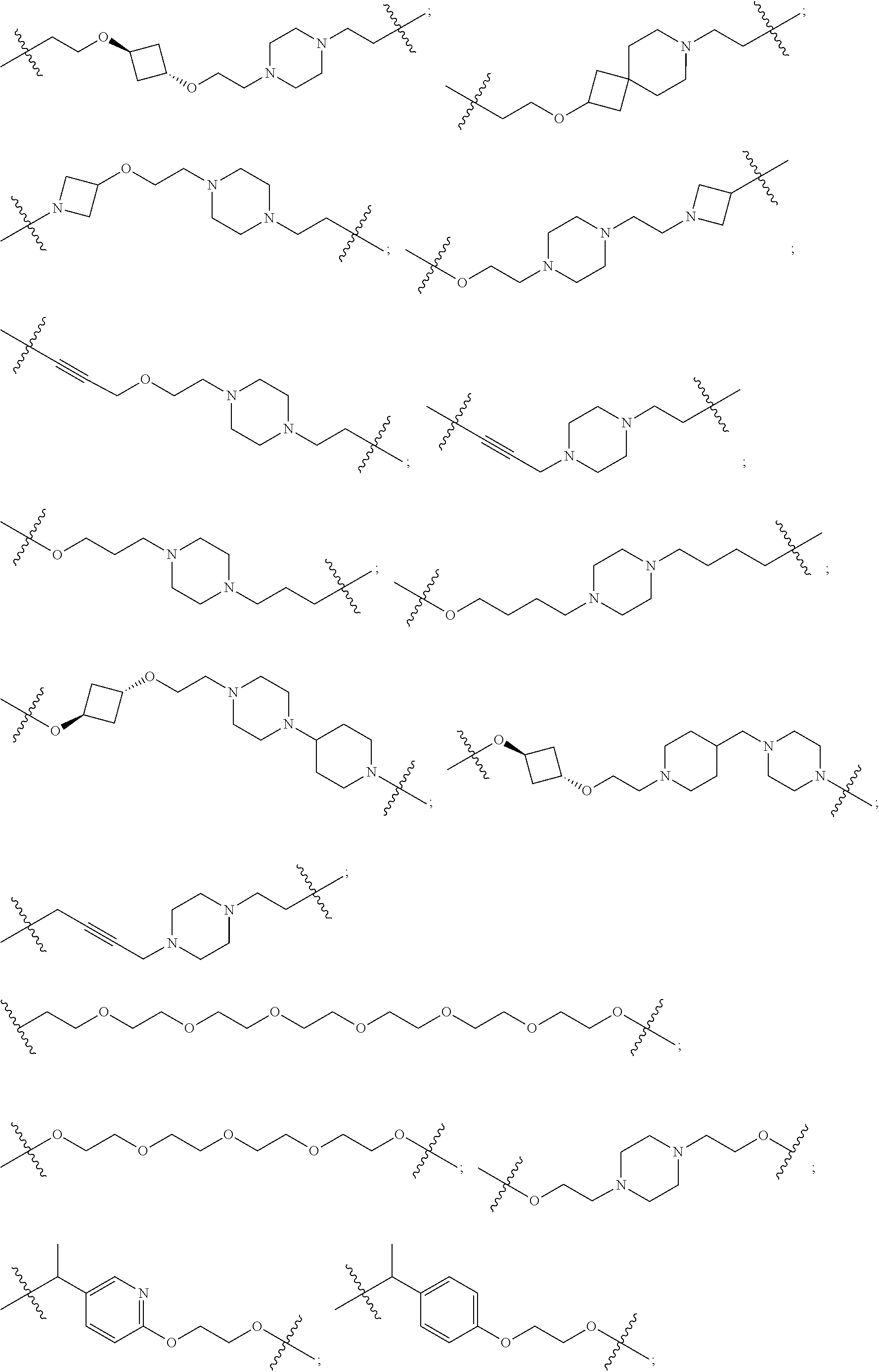

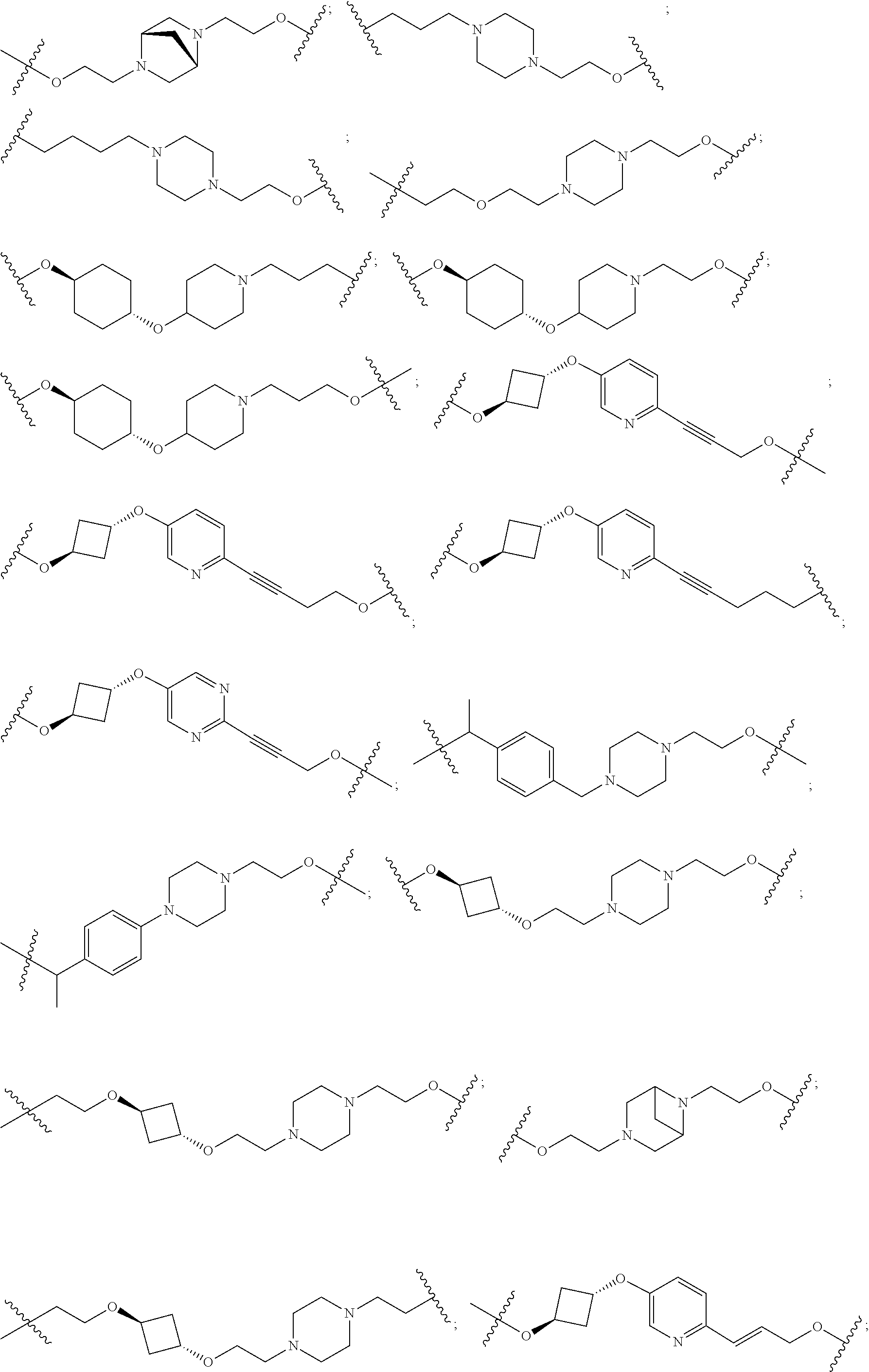

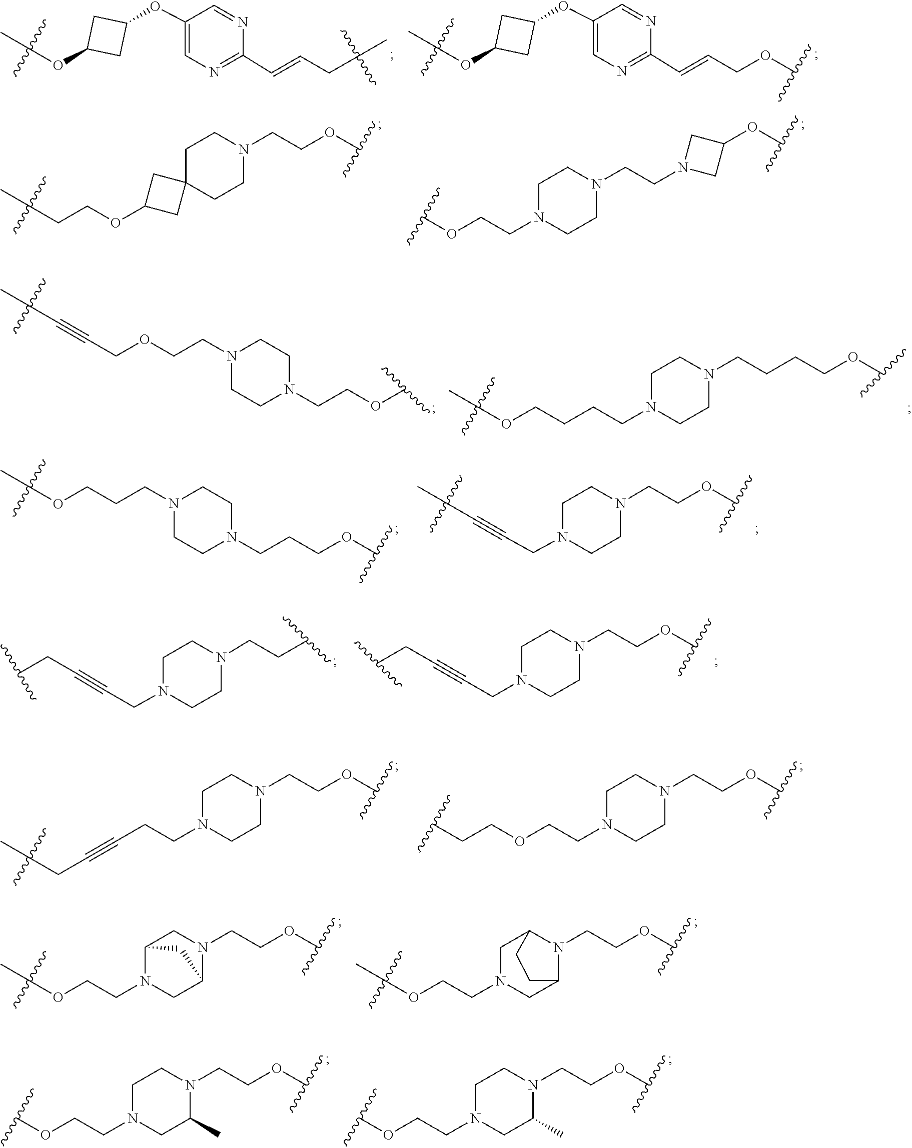

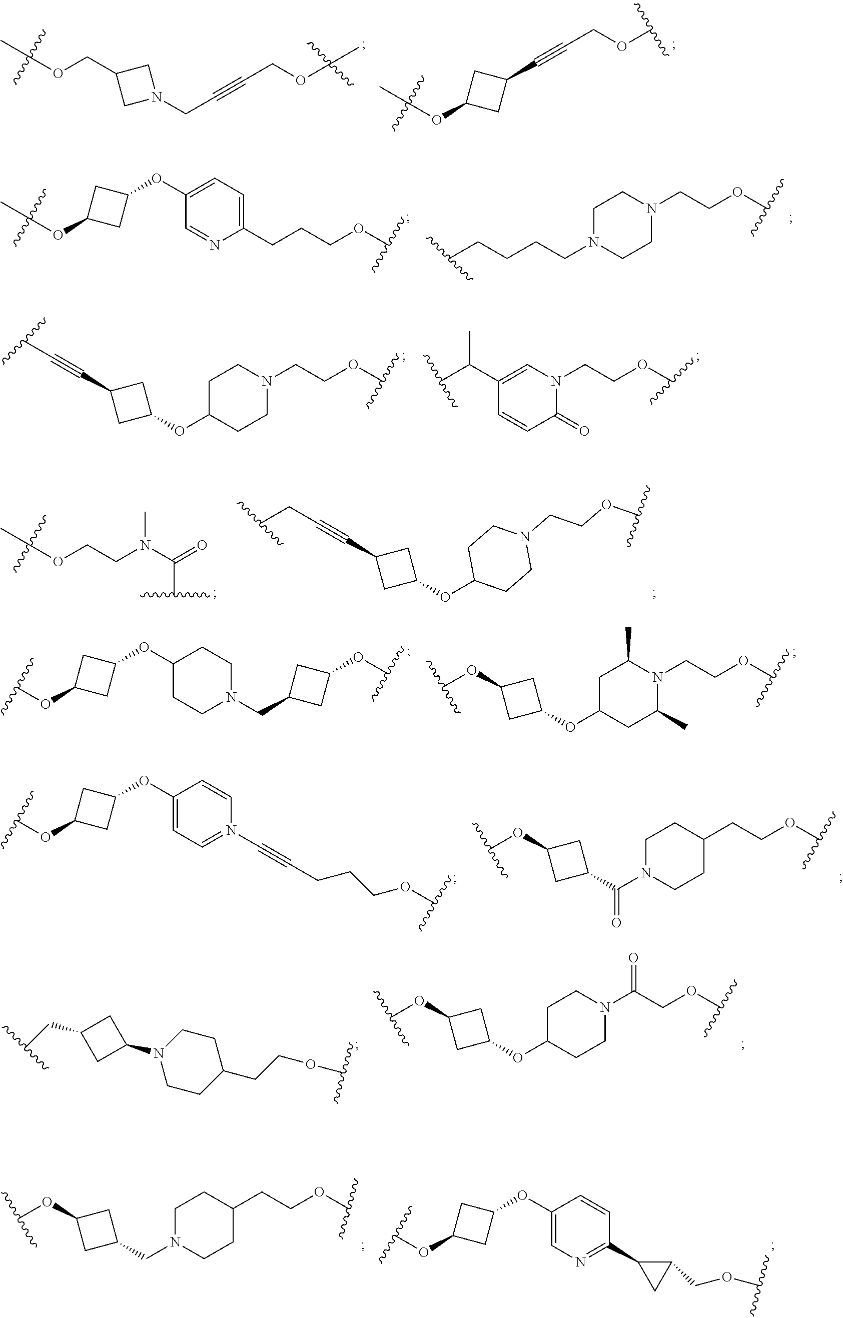

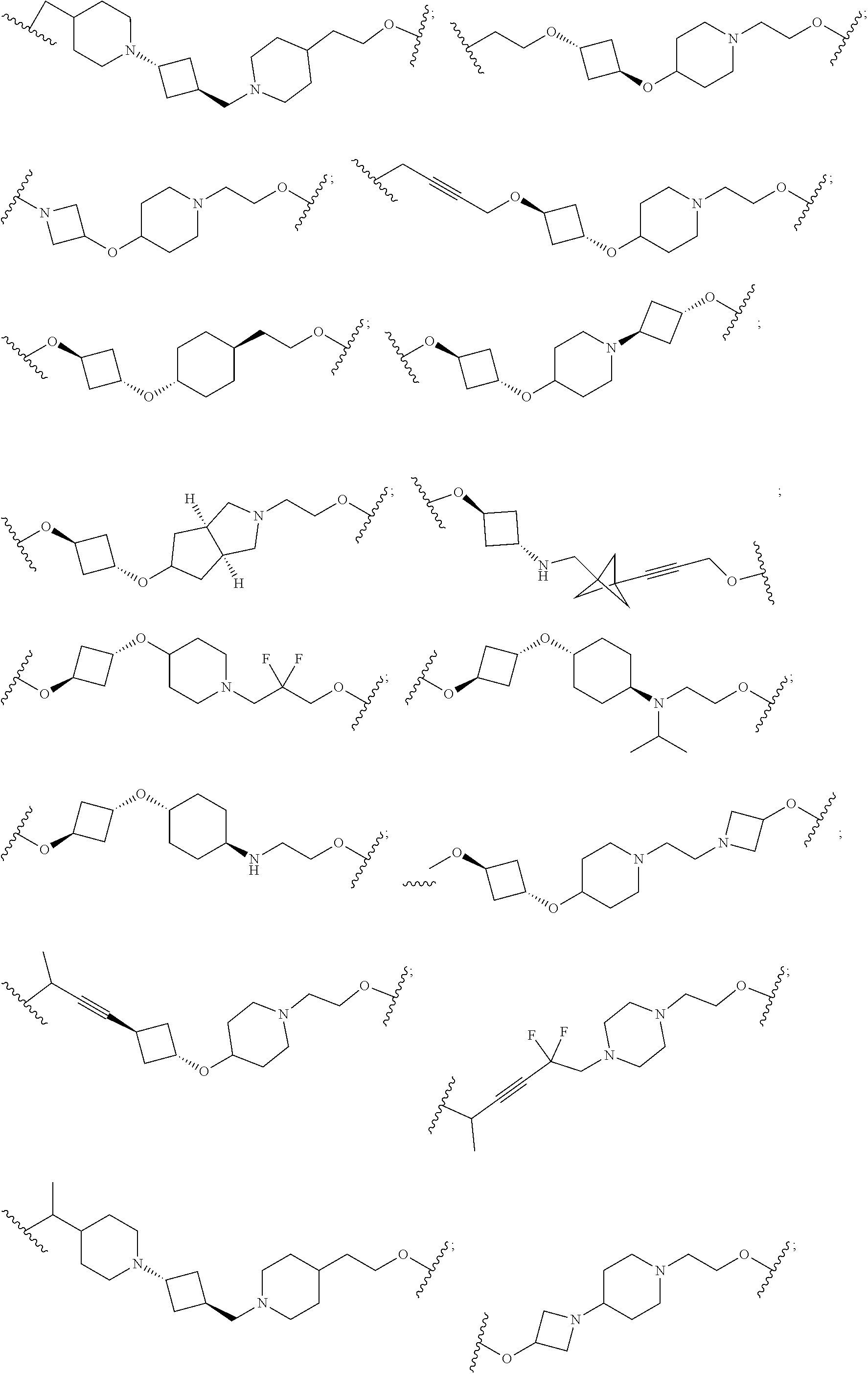

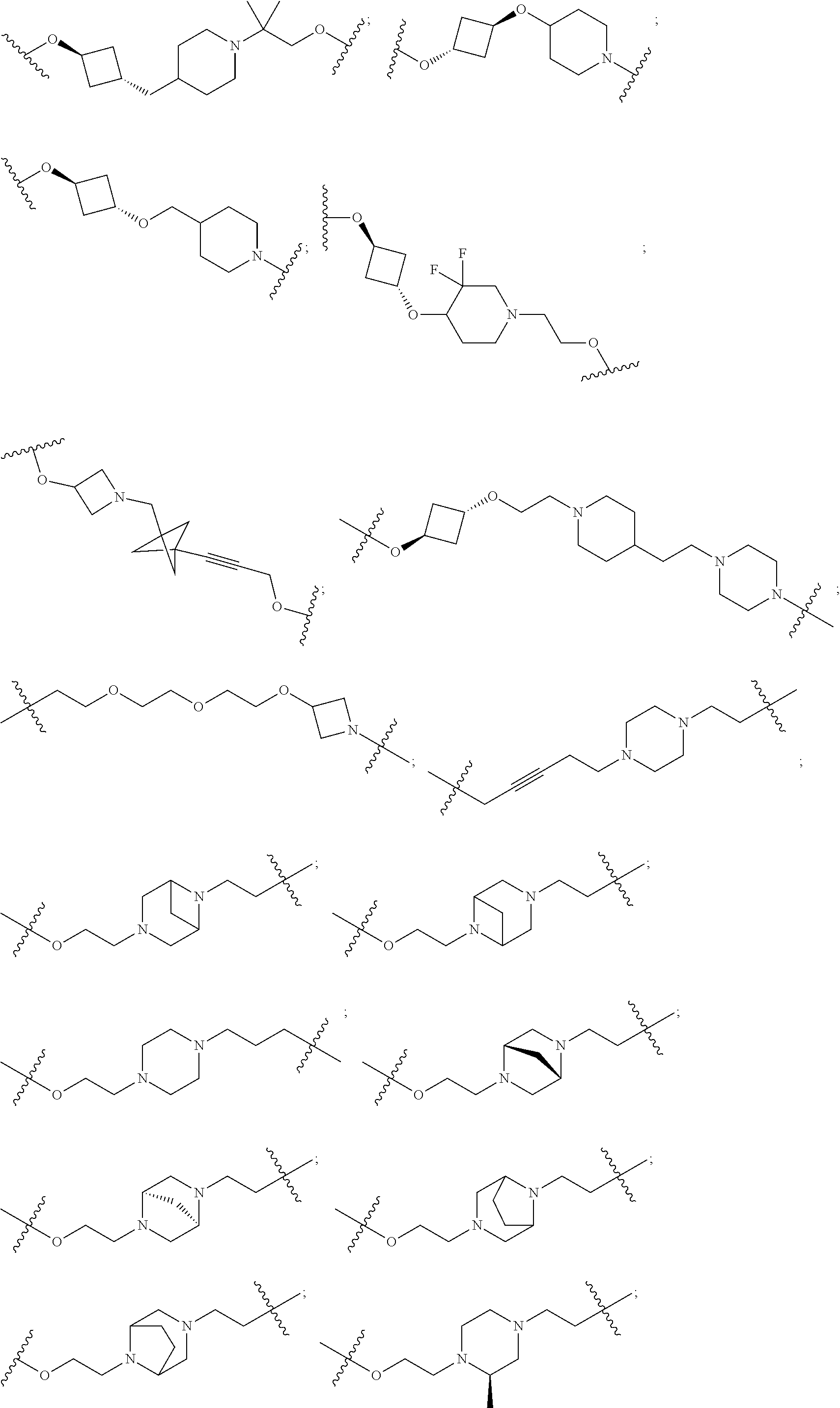

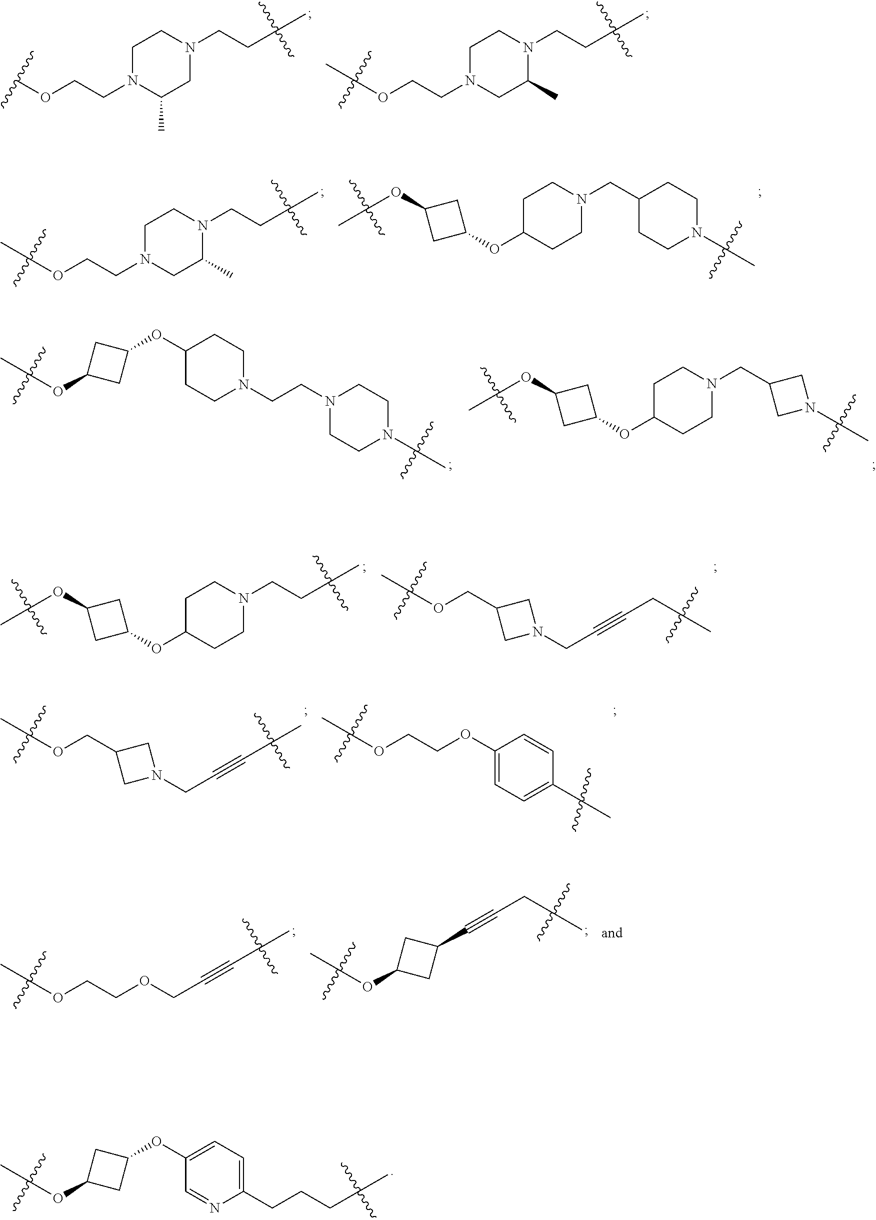

In any aspect or embodiment described herein, the linker unit or linker (L) comprises a group represented by a structure selected from the group consisting of: —O(CH₂)ₘ—O(CH₂)ₙ—O(CH₂)ₒ—O(CH₂)ₚ—O(CH₂)_{q}—O(CH₂)ᵣ—O(CH₂)ₛ—O(CH₂)ₜ—O—(CH₂)ₘ—O(CH₂)ₙ—O(CH₂)ₒ—O(CH₂)ₚ—O(CH₂)_{q}—O(CH₂)ᵣ—O(CH₂)ₛ—O—(CH₂)ₘ—O(CH₂)ₙ—O(CH₂)ₒ—O(CH₂)ₚ—O(CH₂)_{q}—O(CH₂)ᵣ—O(CH₂)ₛ—O(CH₂)ₜ—; —O(CH₂)ₙNCH₃C(O)(CH₂)ₘ—;

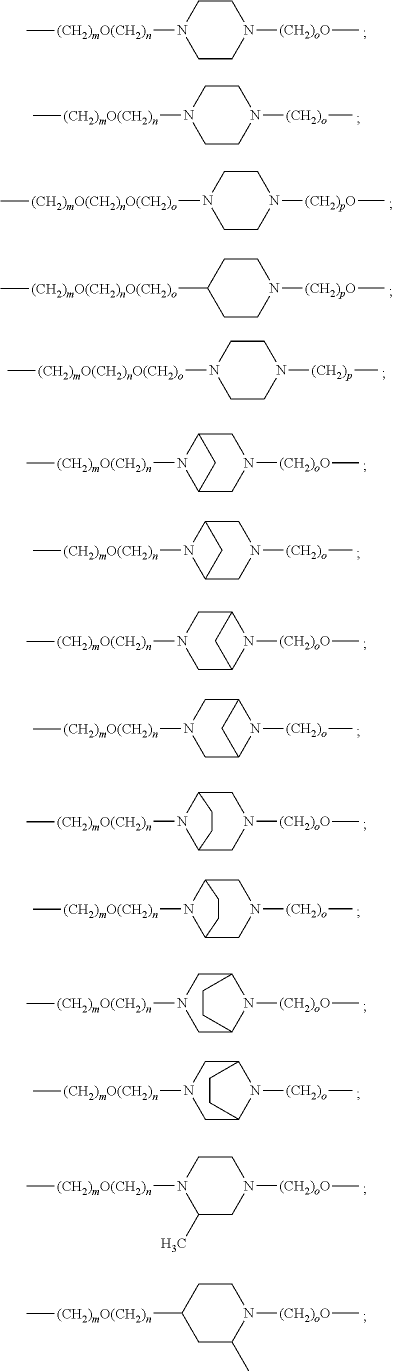

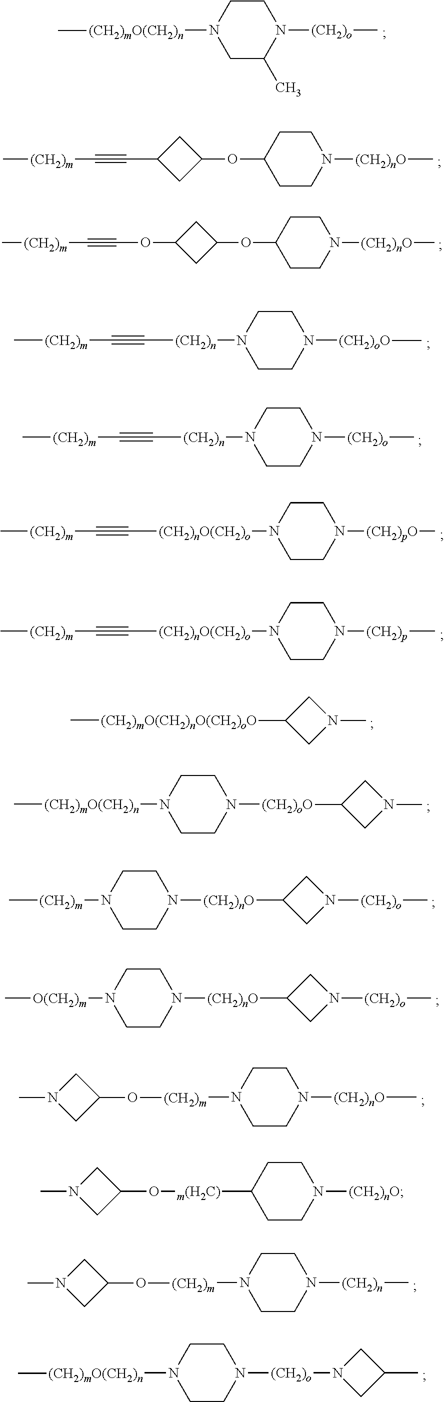

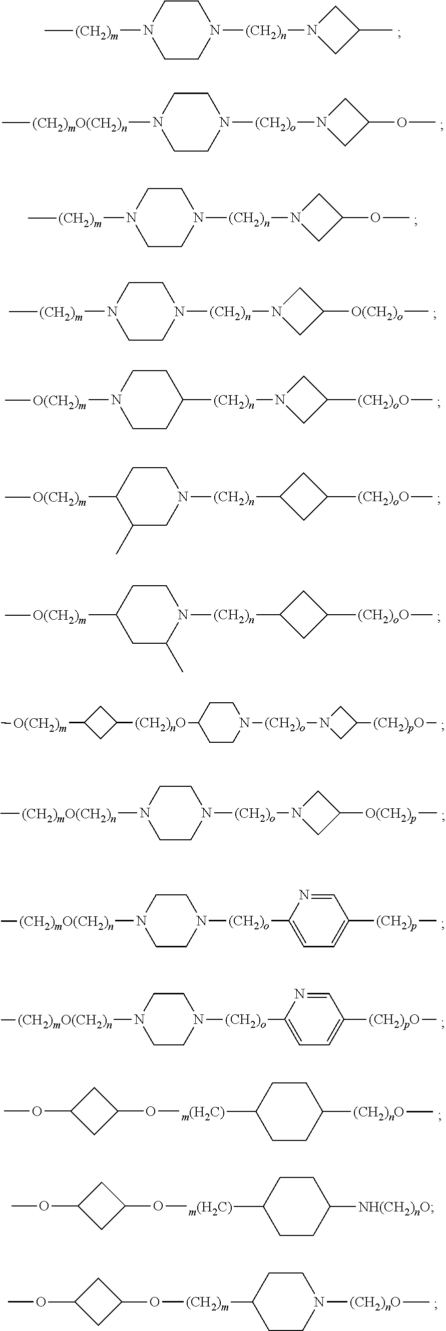

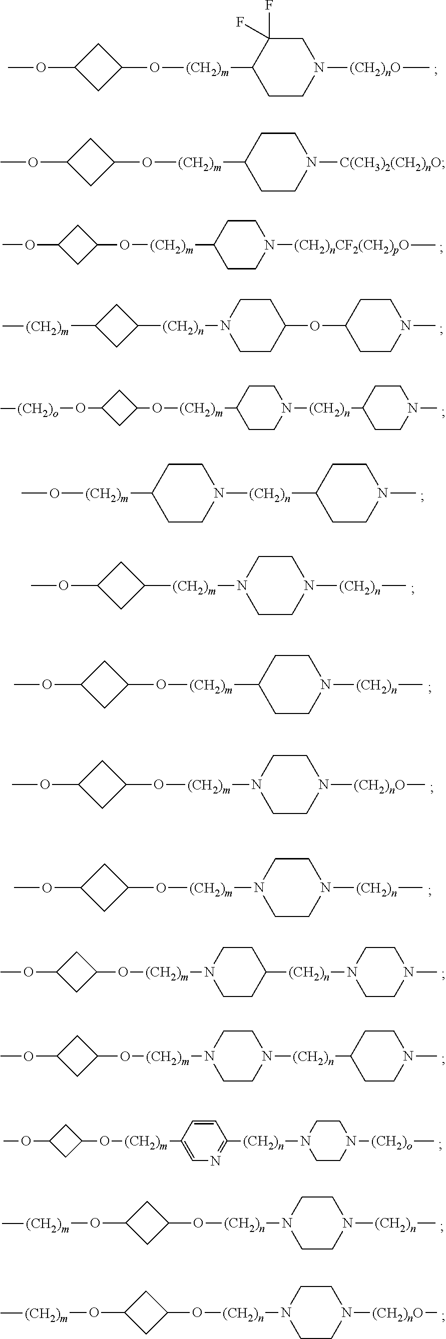

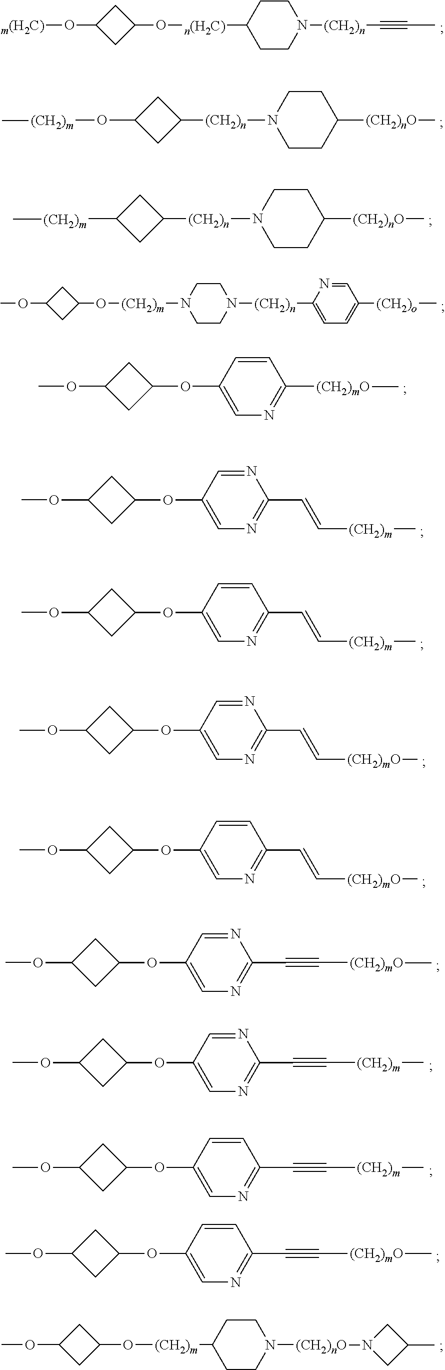

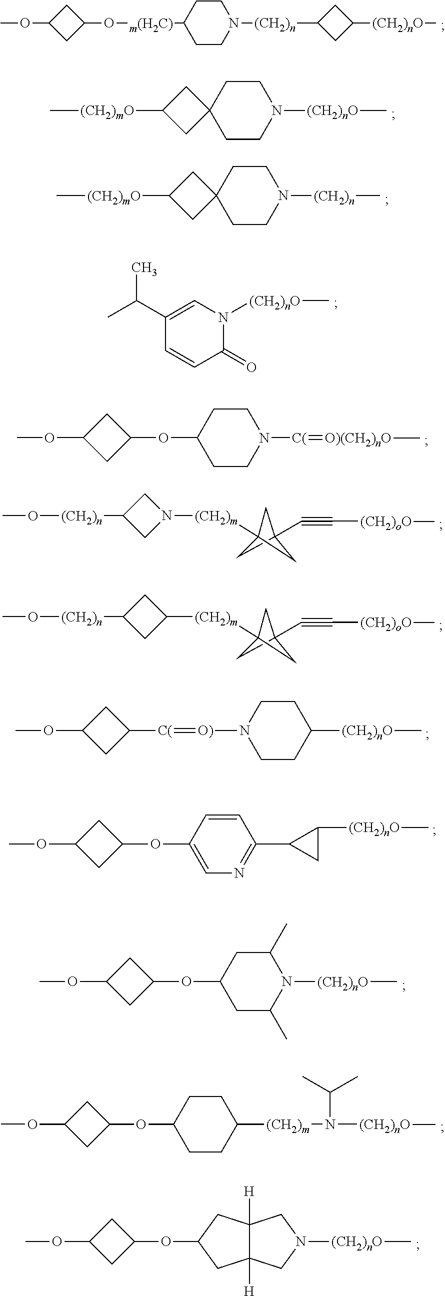

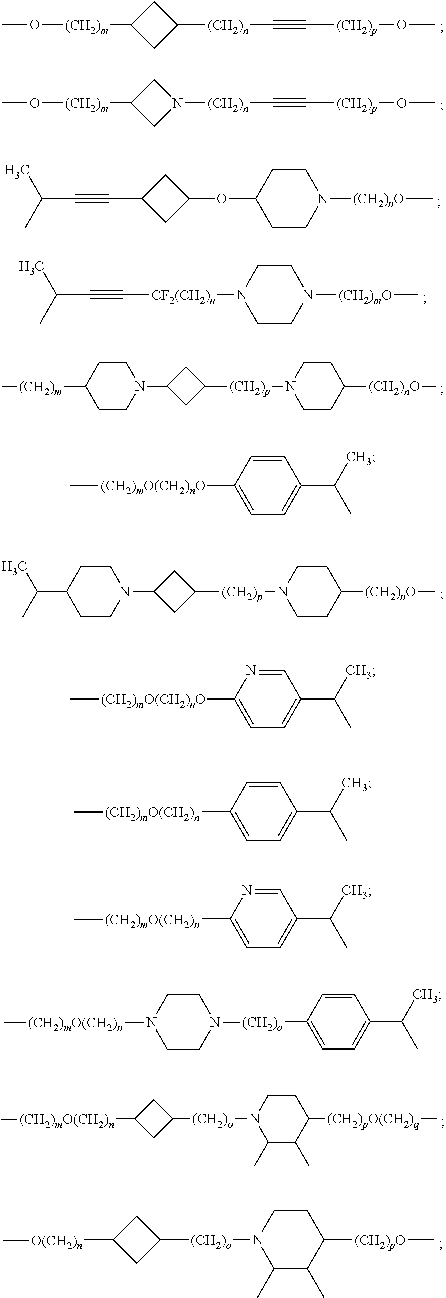

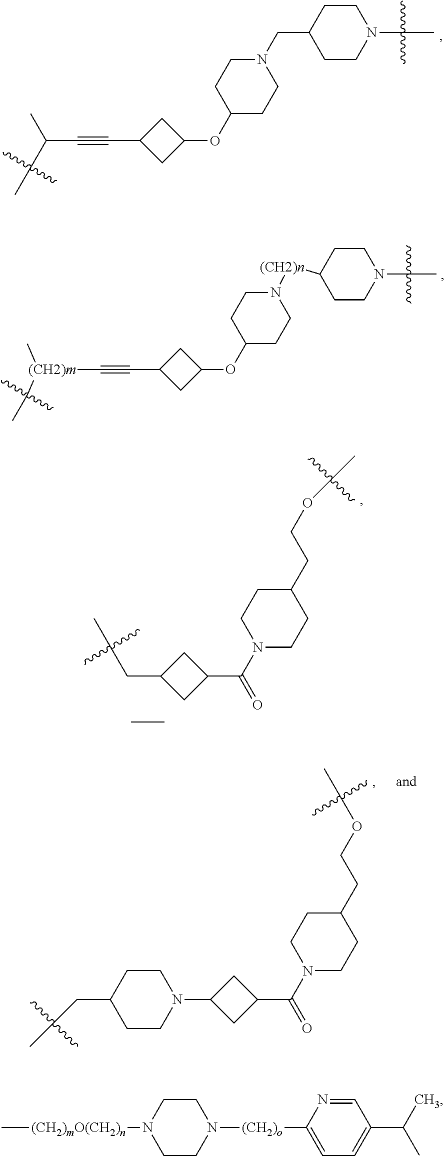

wherein m, n, o, p, q, r, s and t are each independently selected from the integers 0, 1, 2, 3 and 4.

In any aspect or embodiment described herein, the linker (L) is selected from the group consisting of:

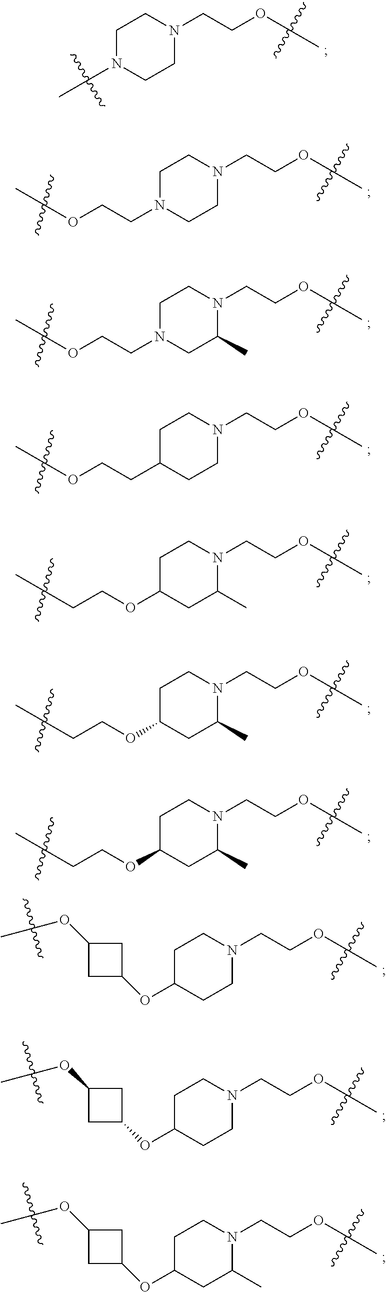

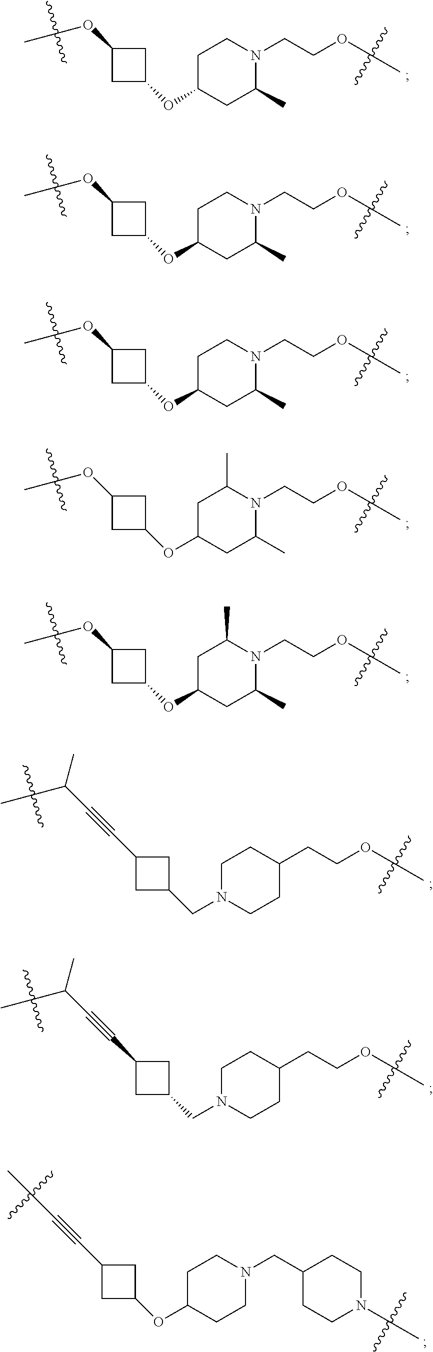

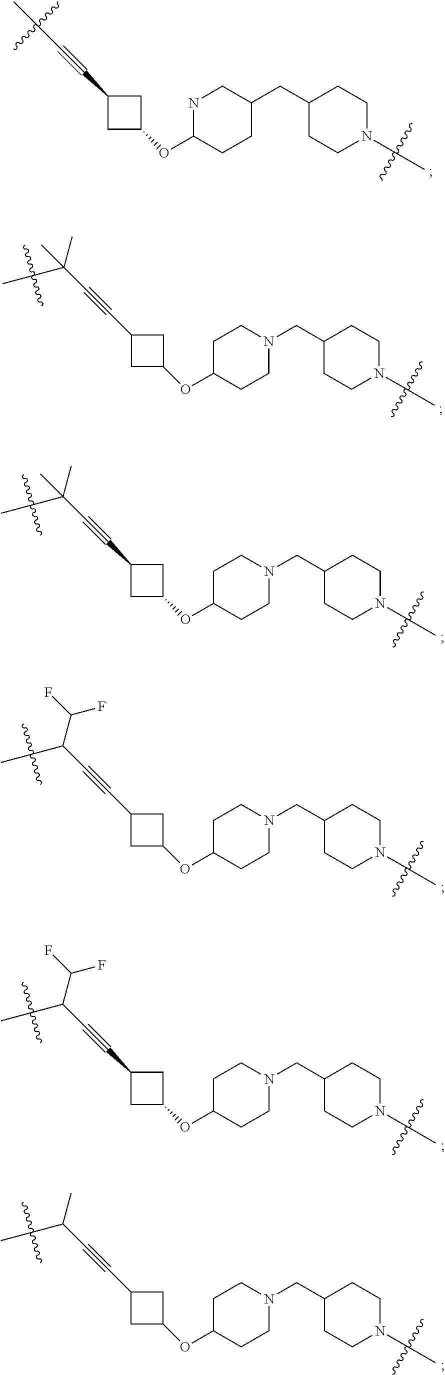 [SMILES: *C#C[C@H]1C[C@H](OC2CCC(CC3CCN(*)CC3)CN2)C1 ; *N1CCC(CN2CCC(OC3CC(C#CC(*)(C)C)C3)CC2)CC1 ; *N1CCC(CN2CCC(O[C@H]3C[C@H](C#CC(*)(C)C)C3)CC2)CC1 ; *C(C#CC1CC(OC2CCN(CC3CCN(*)CC3)CC2)C1)C(F)F ; *C(C#C[C@H]1C[C@H](OC2CCN(CC3CCN(*)CC3)CC2)C1)C(F)F ; *C(C)C#CC1CC(OC2CCN(CC3CCN(*)CC3)CC2)C1]

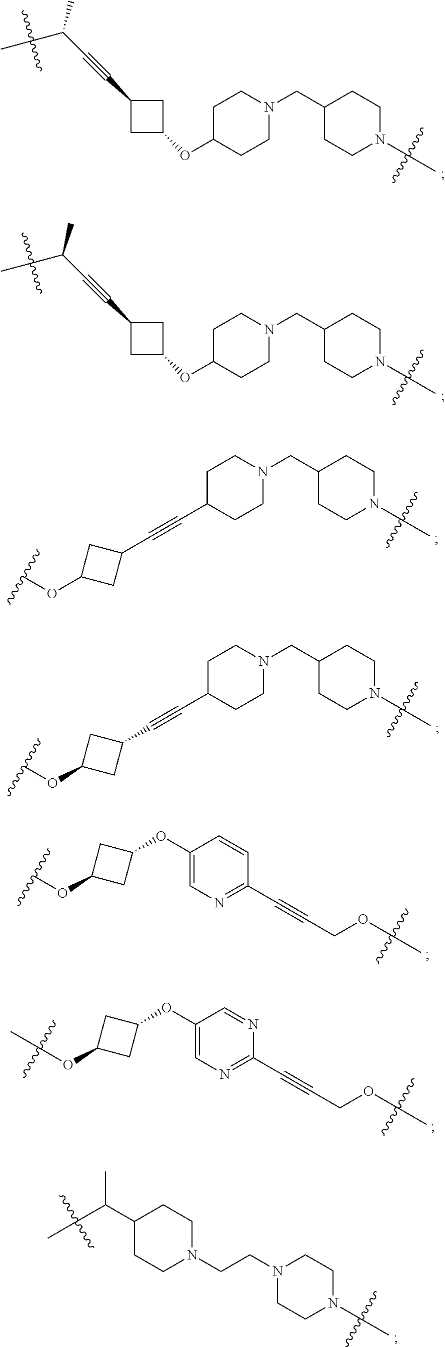

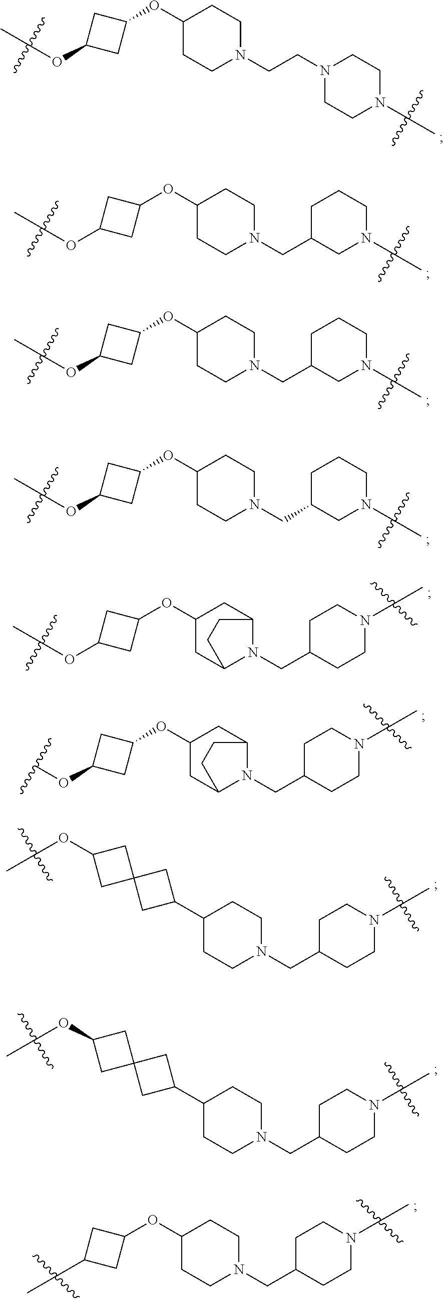

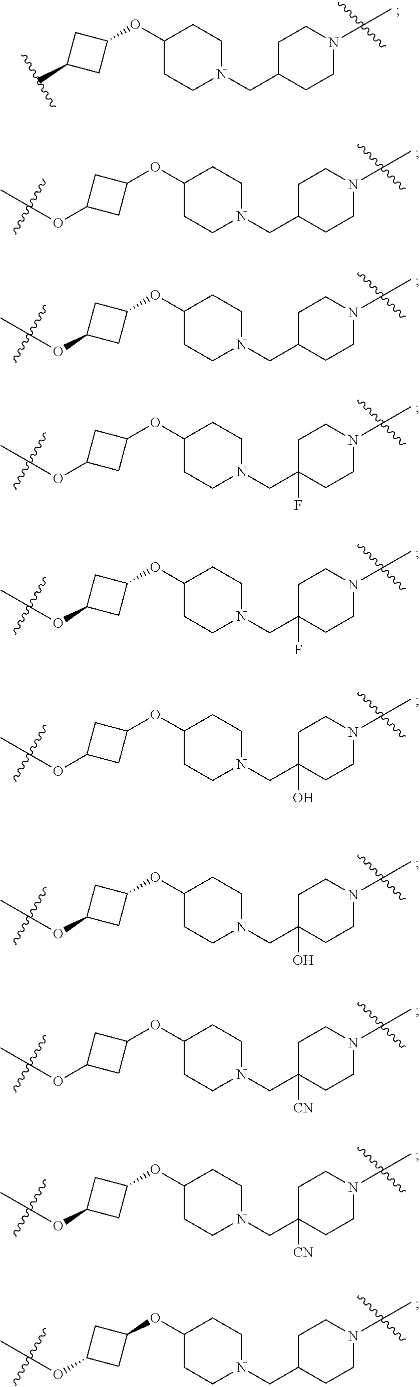

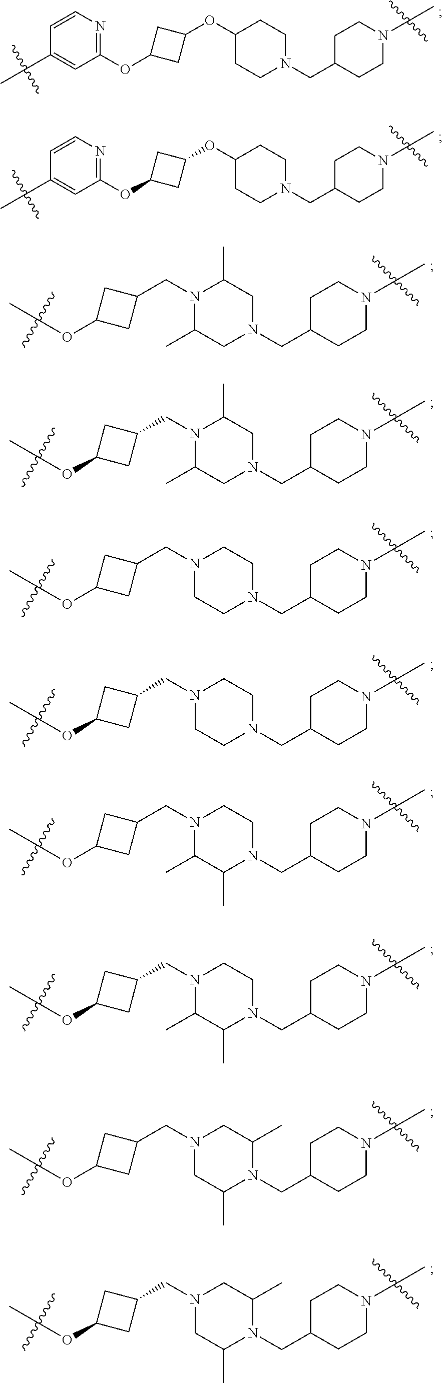

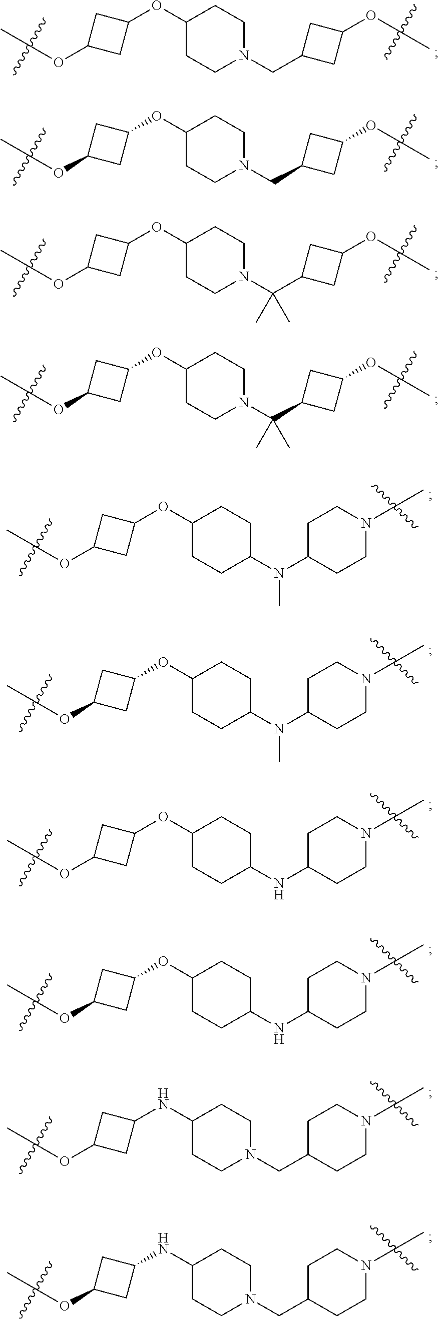

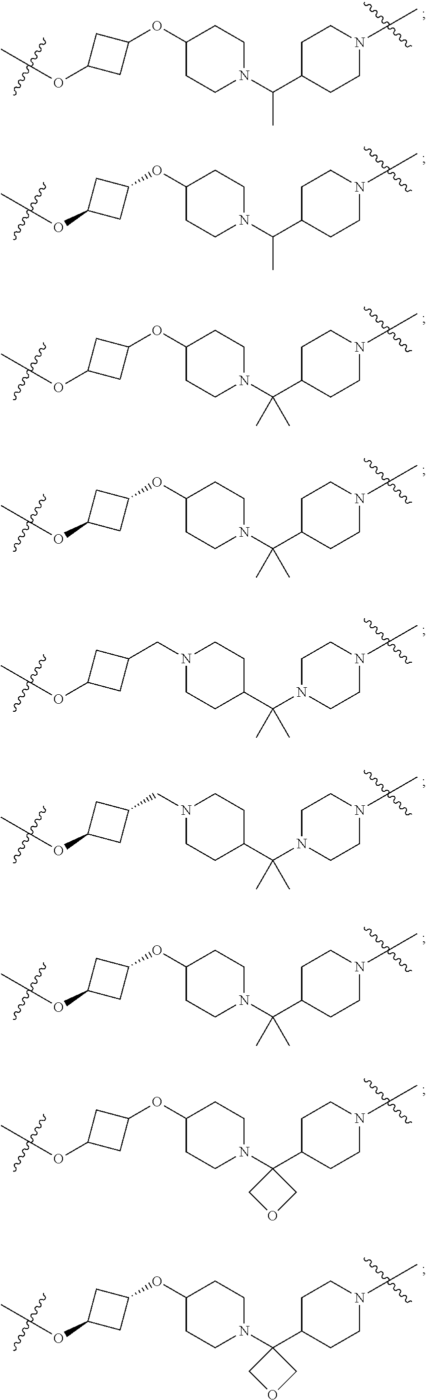

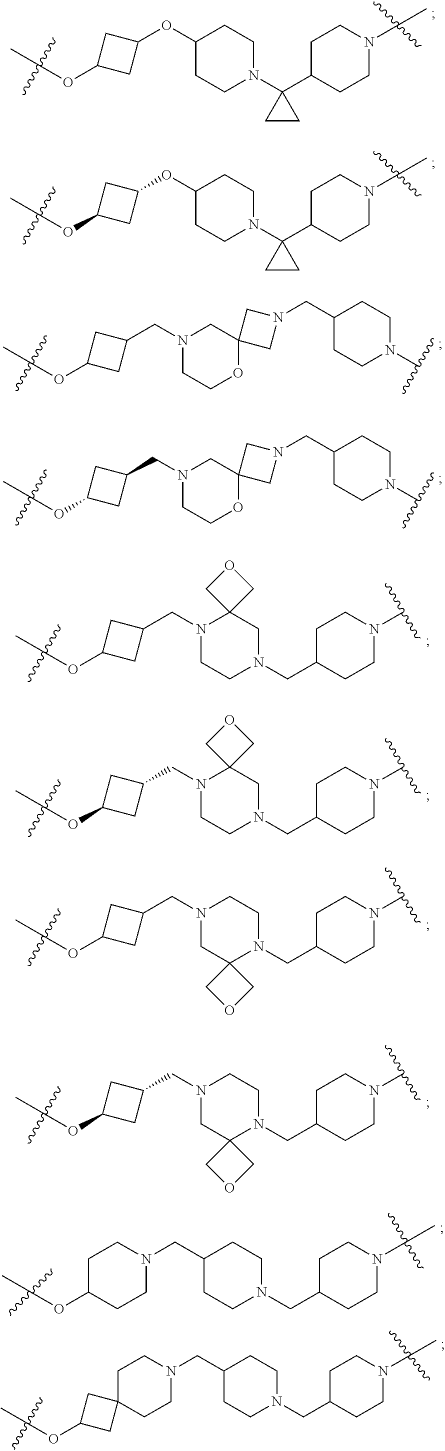

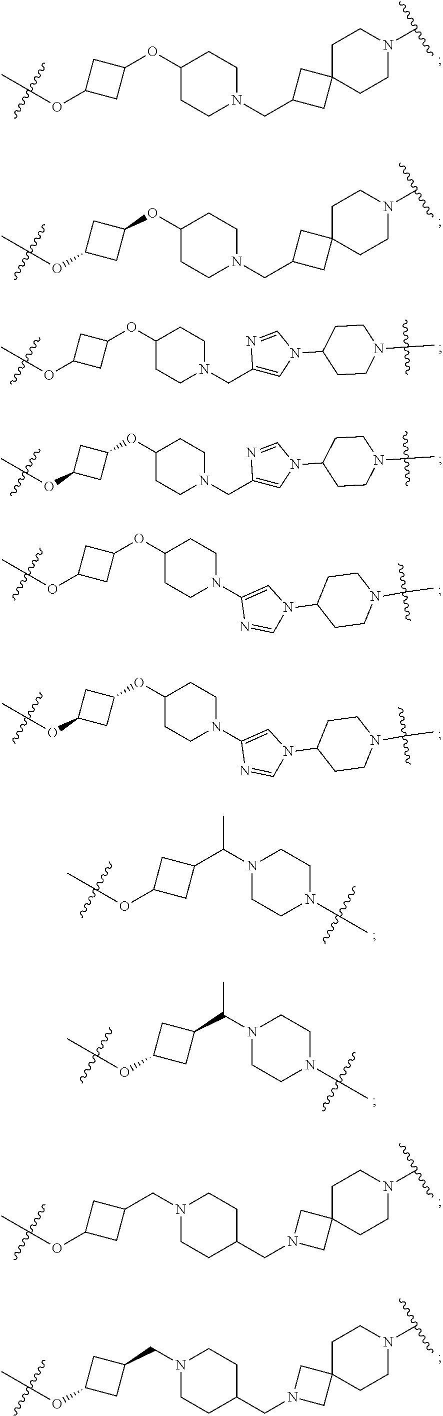

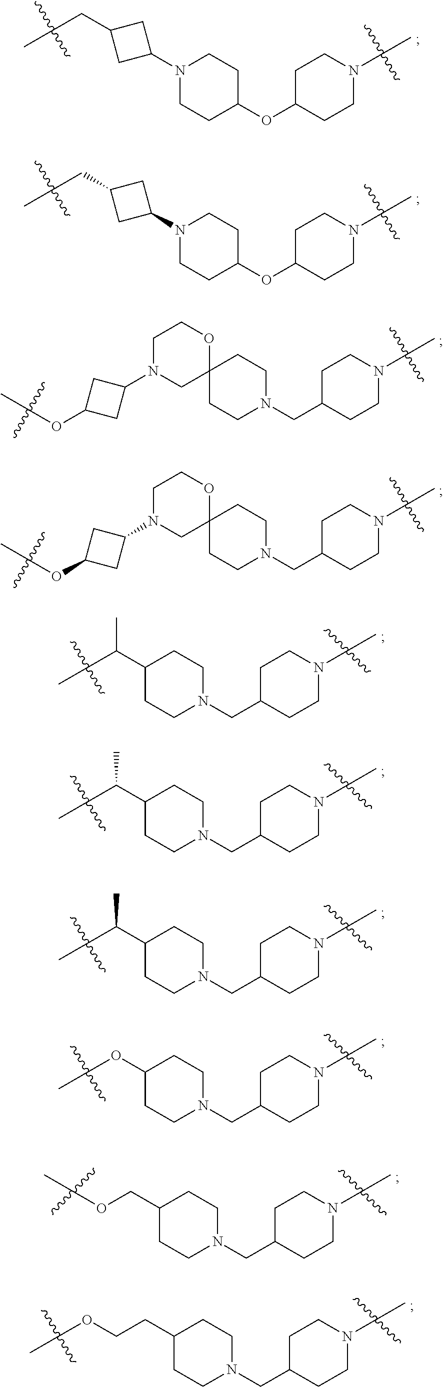

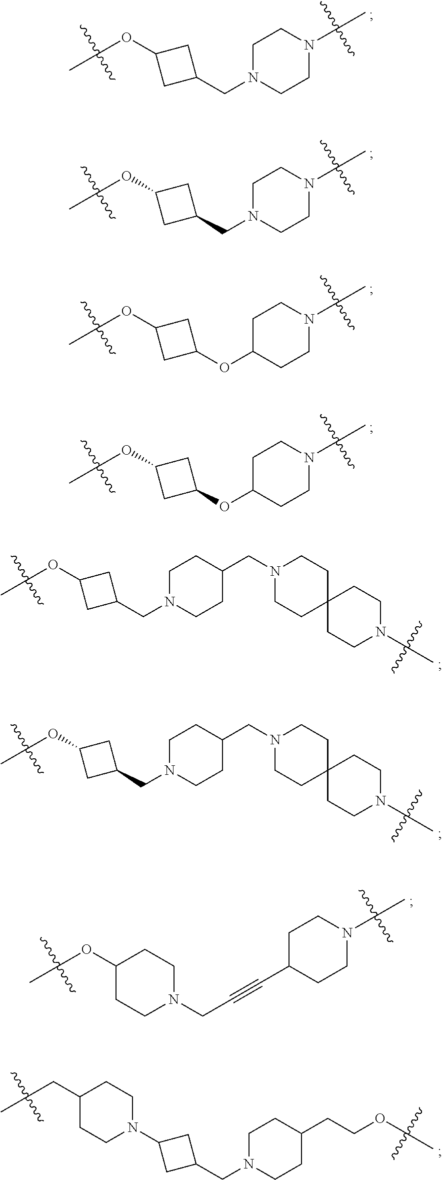

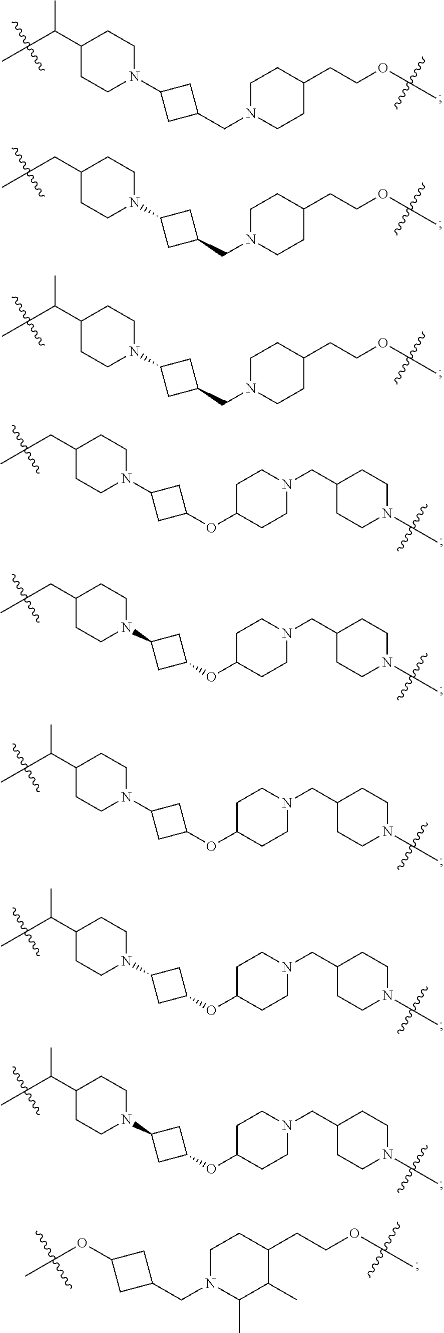

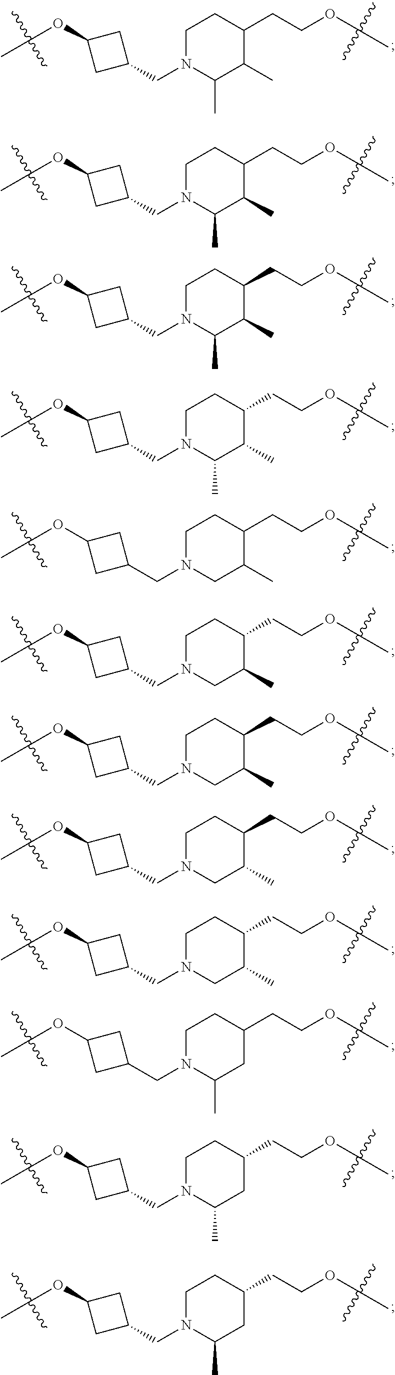

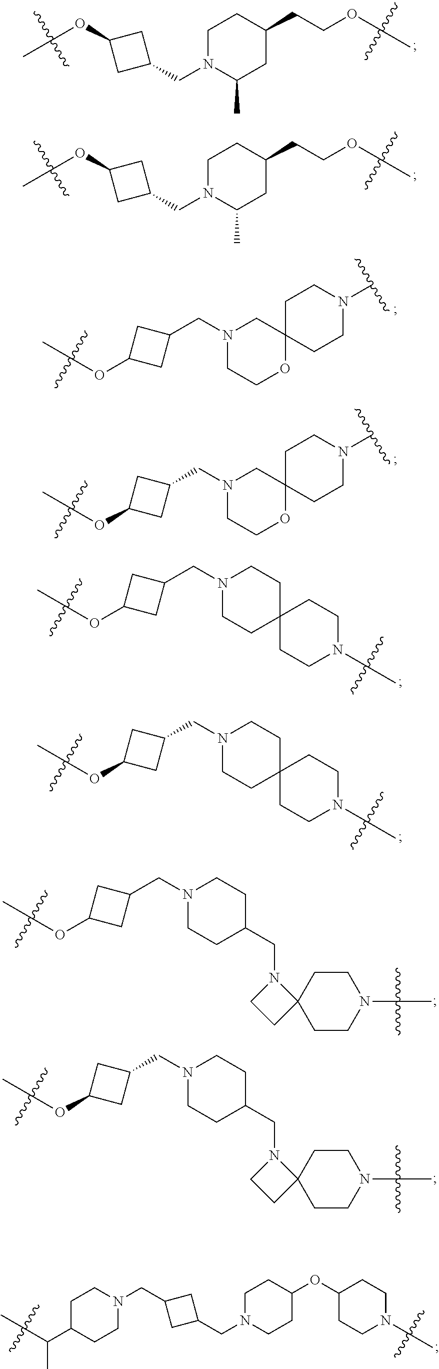

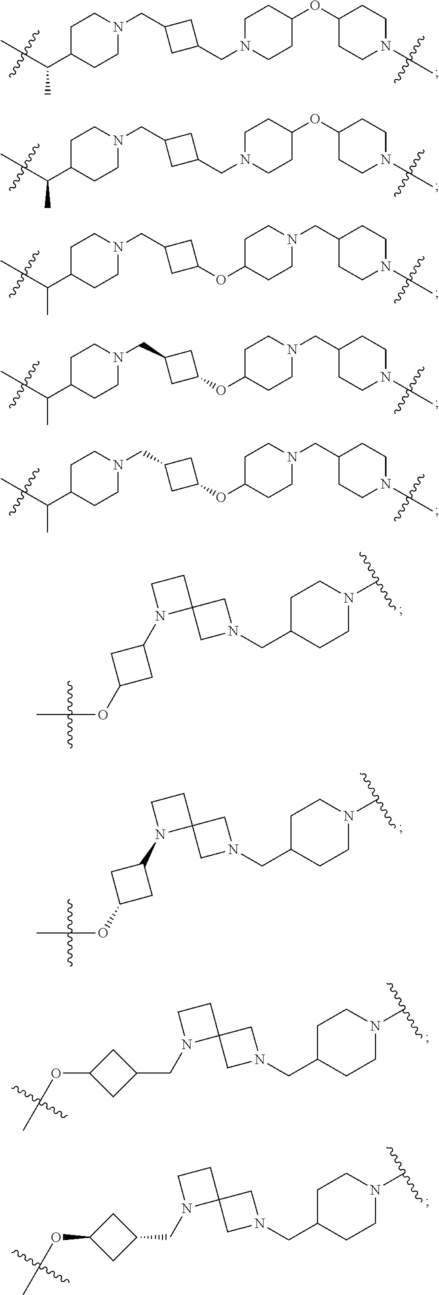

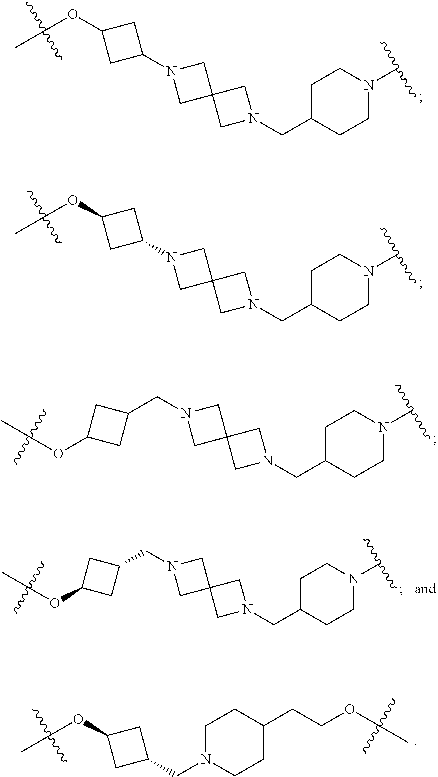 [SMILES: *OC1CC(N2CC3(CN(CC4CCN(*)CC4)C3)C2)C1 ; *O[C@H]1C[C@H](N2CC3(CN(CC4CCN(*)CC4)C3)C2)C1 ; *OC1CC(CN2CC3(CN(CC4CCN(*)CC4)C3)C2)C1 ; *O[C@H]1C[C@H](CN2CC3(CN(CC4CCN(*)CC4)C3)C2)C1 ; *OCCC1CCN(C[C@H]2C[C@H](O*)C2)CC1]

In any aspect or embodiment described herein, the linker (L) is selected from the group consisting of:

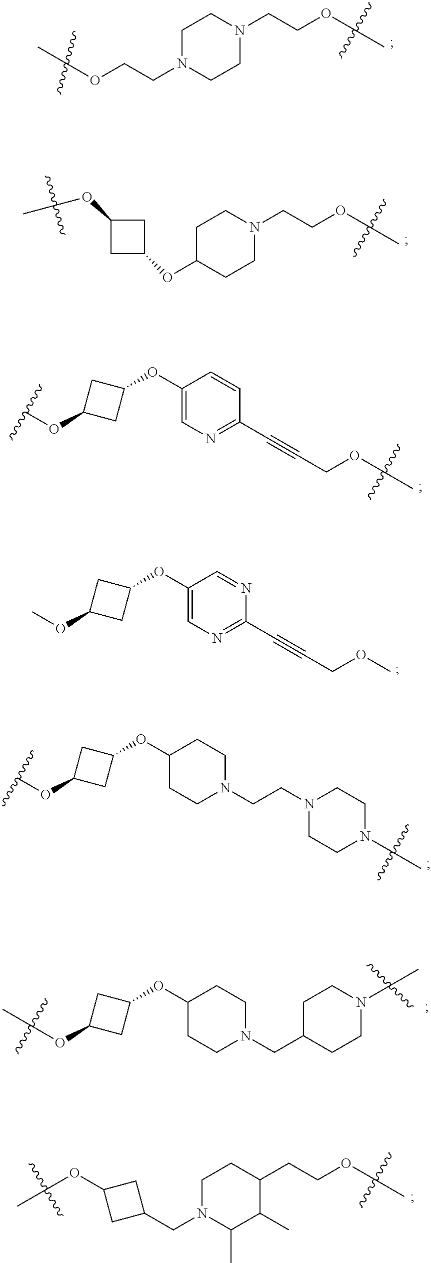

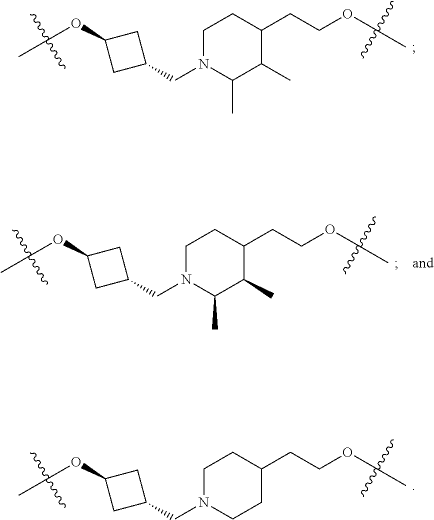 [SMILES: *OCCC1CCN(C[C@H]2C[C@H](O*)C2)C(C)C1C ; *OCCC1CCN(C[C@H]2C[C@H](O*)C2)[C@H](C)[C@@H]1C ; *OCCC1CCN(C[C@H]2C[C@H](O*)C2)CC1]

In additional embodiments, the linker (L) comprises a structure selected from, but not limited to the structure shown below, where a dashed line indicates the attachment point to the PTM or the ULM:

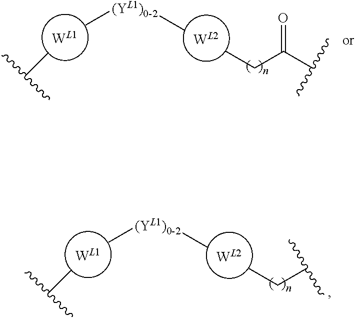

wherein:
W^{L1} and W^{L2} are each independently absent, a 4-8 membered ring with 0-4 heteroatoms, optionally substituted with R^{Q}, each R^{Q} is independently a H, halo, OH, CN, CF₃, optionally substituted linear or branched C₁-C₆ alkyl, optionally substituted linear or branched C₁-C₆ alkoxy, or 2 R^{Q} groups taken together with the atom they are attached to, form a 4-8 membered ring system containing 0-4 heteroatoms;
Y^{L1} is each independently a bond, C₁-C₆ alkyl (linear, branched, optionally substituted) and optionally one or more C atoms are replaced with O; or C₁-C₆ alkoxy (linear, branched, optionally substituted);
n is an integer from 0 to 10; and

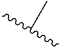

indicates the attachment point to the PTM or the ULM.

In additional embodiments, the linker (L) comprises a structure selected from, but not limited to the structure shown below, where a dashed line indicates the attachment point to the PTM or the ULM:

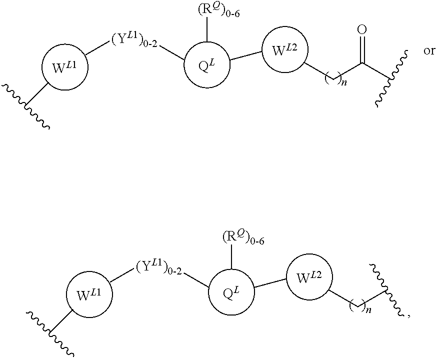

wherein:
W^{L1} and W^{L2} are each independently absent, aryl, heteroaryl, cyclic, heterocyclyl, C₁₋₆ alkyl and optionally one or more C atoms are replaced with O or N, C₁₋₆ alkenyl and optionally one or more C atoms are replaced with O, C₁₋₆ alkynyl and optionally one or more C atoms are replaced with O, bicyclic, biaryl, biheteroaryl, or biheterocyclyl, each optionally substituted with R^{Q}, each R^{Q} is independently a H, halo, OH, CN, CF₃, hydroxyl, nitro, C≡CH, C₂₋₆ alkenyl, C₂₋₆ alkynyl, optionally substituted linear or branched C₁-C₆ alkyl, optionally substituted linear or branched C₁-C₆ alkoxy, optionally substituted OC₁₋₃alkyl (e.g., optionally substituted by 1 or more —F), OH, NH₂, NR^{Y1}R^{Y2}, CN, or 2 R^{Q} groups taken together with the atom they are attached to, form a 4-8 membered ring system containing 0-4 heteroatoms;
Y^{L1} is each independently a bond, NR^{YL1}, O, S, NR^{YL2}, CR^{YL1}R^{YL2}, C═O, C=S, SO, SO₂, C₁-C₆ alkyl (linear, branched, optionally substituted) and optionally one or more C atoms are replaced with O; C₁-C₆ alkoxy (linear, branched, optionally substituted);
Q^{L} is a 3-6 membered alicyclic or aromatic ring with 0-4 heteroatoms, optionally bridged, optionally substituted with 0-6 R^{Q}, each R^{Q} is independently H, linear or branched C₁₋₆ alkyl optionally substituted by 1 or more halo or C₁₋₆ alkoxyl, or 2 R^{Q} groups taken together with the atom they are attached to, form a 3-8 membered ring system containing 0-2 heteroatoms);
R^{YL1}, R^{YL2} are each independently H, OH, C₁₋₆ alkyl (linear, branched, optionally substituted by 1 or more halo, C₁₋₆ alkoxyl), or R¹, R² together with the atom they are attached to, form a 3-8 membered ring system containing 0-2 heteroatoms);
n is an integer from 0 to 10; and

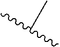

indicates the attachment point to the PTM or the ULM.

In additional embodiments, the linker group is optionally substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocyclyl group. In certain embodiments, the linker may be asymmetric or symmetrical.

In any of the embodiments of the compounds described herein, the linker group may be any suitable moiety as described herein. In one embodiment, the linker is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units.

In another embodiment, the present disclosure is directed to a compound which comprises a PTM group as described above, which binds to a target protein or polypeptide (e.g., SMARCA2, BRAHMA or BRM), which is ubiquitinated by a ubiquitin ligase and is chemically linked directly to the ULM group or through a linker moiety L, or PTM is alternatively a ULM′ group which is also a ubiquitin ligase binding moiety, which may be the same or different than the ULM group as described above and is linked directly to the ULM group directly or through the linker moiety; and L is a linker moiety as described above which may be present or absent and which chemically (covalently) links ULM to PTM, or a pharmaceutically acceptable salt, enantiomer, stereoisomer, solvate or polymorph thereof.

In certain embodiments, the linker group L is a group comprising one or more covalently connected structural units independently selected from the group consisting of:

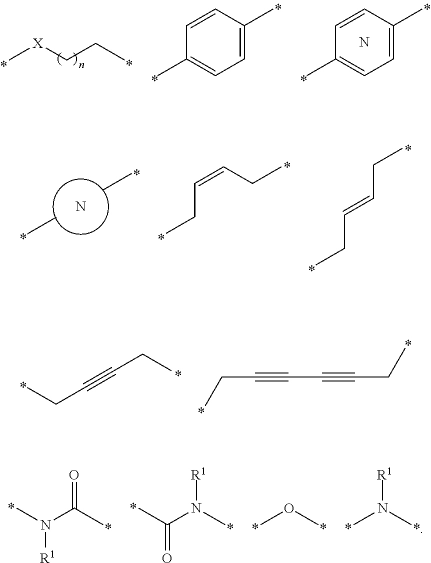

The X is selected from the group consisting of O, N, S, S(O) and SO₂; n is integer from 1 to 5; R^{L1} is hydrogen or alkyl,

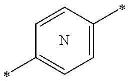

is a mono- or bicyclic aryl or heteroaryl optionally substituted with 1-3 substituents selected from alkyl, halogen, haloalkyl, hydroxy, alkoxy or cyano;

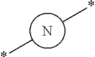

is a mono- or bicyclic cycloalkyl or a heterocyclyl optionally substituted with 1-3 substituents selected from alkyl, halogen, haloalkyl, hydroxy, alkoxy or cyano; and the phenyl ring fragment can be optionally substituted with 1, 2 or 3 substituents selected from the group consisting of alkyl, halogen, haloalkyl, hydroxy, alkoxy and cyano. In an embodiment, the linker group L comprises up to 10 covalently connected structural units, as described above.

Although the ULM group and PTM group may be covalently linked to the linker group through any group which is appropriate and stable to the chemistry of the linker, in preferred aspects of the present disclosure, the linker is independently covalently bonded to the ULM group and the PTM group preferably through an amide, ester, thioester, keto group, carbamate (urethane), carbon or ether, each of which groups may be inserted anywhere on the ULM group and PTM group to provide maximum binding of the ULM group on the ubiquitin ligase and the PTM group on the target protein to be degraded. (It is noted that in certain aspects where the PTM group is a ULM group, the target protein for degradation may be the ubiquitin ligase itself). In certain preferred aspects, the linker may be linked to an optionally substituted alkyl, alkylene, alkenyl or alkynyl group, an aryl group or a heterocyclyl group on the ULM and/or PTM groups.

### Exemplary PTMs

In any aspect or embodiment of the present disclosure, the PTM group is a moiety, which binds to target proteins, such as Switch/Sucrose Non Fermentable (SWI/SNF)-Related, Matrix-Associated, Actin-Dependent Regulator of Chromatin, Subfamily A, Member 2 (SMARCA2) or BRM. Thus, in any aspect or embodiment described herein, the PTM group is any moiety that binds to SMARCA2 or BRM protein specifically (binds to the target protein SMARCA2, BRAHMA or BRM).

In certain embodiments, the compounds as described herein include a means for binding a target protein, e.g., Brm. As such, in certain aspects, the disclosure provides a bifunctional compound having a means for binding Brm, and a means for binding VHL and a means for chemically coupling the means for binding Brm to the means for binding VHL.

The compositions described below exemplify some of the members of small molecule target protein binding moieties. Such small molecule target protein binding moieties also include pharmaceutically acceptable salts, enantiomers, solvates and polymorphs of these compositions, as well as other small molecules that may target SMARCA2. These binding moieties are linked to the ubiquitin ligase binding moiety preferably through a linker in order to present a target protein (to which the protein target moiety is bound) in proximity to the ubiquitin ligase for ubiquitination and degradation. Any protein (e.g., SMARCA2, BRAHMA or BRM), which can bind to a protein target moiety or PTM group and acted on or degraded by a ubiquitin ligase is a target protein according to the present disclosure.

The present disclosure may be used to treat a number of disease states and/or conditions; including any disease state and/or condition in which proteins are dysregulated (e.g., SMARCA4-deficiency/mutation) and where a patient would benefit from the degradation and/or inhibition of proteins, such as SMARCA2, BRAHMA or BRM.

In an additional aspect, the description provides therapeutic compositions comprising an effective amount of a compound as described herein or salt form thereof, and a pharmaceutically acceptable carrier, additive or excipient, and optionally an additional bioactive agent. The therapeutic compositions modulate protein degradation in a patient or subject, for example, an animal such as a human, and can be used for treating or ameliorating disease states or conditions which are modulated through the degraded protein. In certain embodiments, the therapeutic compositions as described herein may be used to effectuate the degradation of proteins of interest for the treatment or amelioration of a disease, e.g., cancer such as at least one of a SWI/SNF associated cancer, a SMARCA4-mutation associated cancer, a SMARCA4-deficient cancer, or a cancer with decreased expression of SMARCA4 relative to normal SMARCA4 expression (e.g., decreased expression relative to the expression of non-mutated SMARCA4 or SMARCA4 in a similarly situated non-cancerous cell with wildtype SMARCA4), including lung cancer or non-small cell lung cancer. In any aspect or embodiment described herein, the disease is at least one of SWI/SNF associated cancer, a cancer with a SMARCA4 mutation, a cancer with a SMARCA4-deficiency, or a combination thereof, which may be lung cancer or a non-small cell lung cancer.

In certain additional embodiments, the therapeutic compositions as described herein may be used to effectuate the degradation of proteins of interest for the treatment or amelioration of a disease, e.g., cancer such as at least one of a SWI/SNF associated cancer, a SMARCA2-associated cancer or a cancer with normal or over-expression of SMARCA2.

In alternative aspects, the present disclosure relates to a method for treating a disease state or ameliorating the symptoms of a disease or condition in a subject in need thereof by degrading a protein or polypeptide through which a disease state or condition is modulated comprising administering to said patient or subject an effective amount, e.g., a therapeutically effective amount, of at least one compound as described hereinabove, optionally in combination with a pharmaceutically acceptable carrier, additive or excipient, and optionally an additional bioactive agent, wherein the composition is effective for treating or ameliorating the disease or disorder or symptom thereof in the subject. The method according to the present disclosure may be used to treat a large number of disease states or conditions including cancer, by virtue of the administration of effective amounts of at least one compound described herein. The disease state or condition may be a disease caused by a microbial agent or other exogenous agent such as a virus, bacteria, fungus, protozoa or other microbe or may be a disease state, which is caused by overexpression of a protein, which leads to a disease state and/or condition.

In another aspect, the description provides methods for identifying the effects of the degradation of proteins of interest in a biological system using compounds according to the present disclosure.

The term “target protein” is used to describe a protein or polypeptide, which is a target for binding to a compound according to the present disclosure and degradation by ubiquitin ligase hereunder. Such small molecule target protein binding moieties also include pharmaceutically acceptable salts, enantiomers, solvates and polymorphs of these compositions, as well as other small molecules that may target a protein of interest. These binding moieties are linked to at least one ULM group (e.g. VLM) through at least one linker group L.

The protein target may be used in screens that identify compound moieties which bind to the protein and by incorporation of the moiety into compounds according to the present disclosure, the level of activity of the protein may be altered for therapeutic end result.

The term “protein target moiety” or PTM is used to describe a small molecule which binds to a target protein or other protein or polypeptide of interest, such as SMARCA2 or BRM, and places/presents that protein or polypeptide in proximity to an ubiquitin ligase such that degradation of the protein or polypeptide by ubiquitin ligase may occur. The compositions described below exemplify some of the members of the small molecule target proteins.

In any aspect or embodiment described herein, the PTM of the present disclosure has a chemical structure represented by:

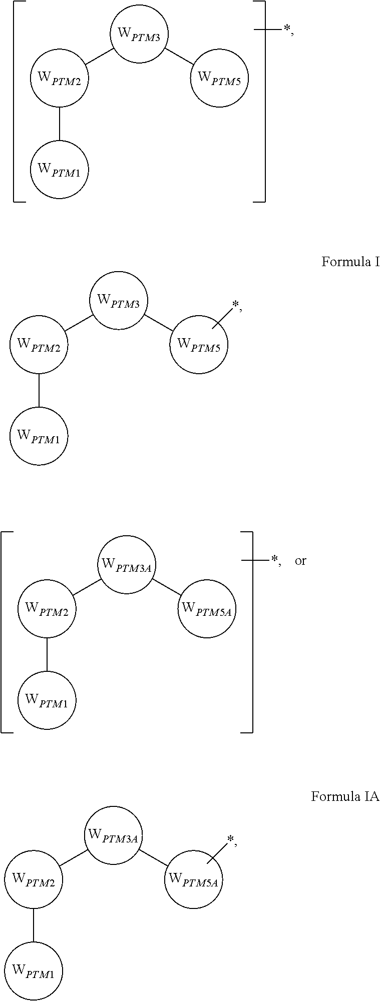

wherein:
W_{PTM1} is an optionally substituted 5-6-membered aryl or heteroaryl ring (e.g., a 5-6 member aryl or heteroaryl substituted with 0, 1, 2, or 3 substituents selected from hydroxy, halogen, alkoxy, alkyl, haloalkyl, phosphate, amino, alkylamino, cyano or combination thereof);
W_{PTM2} is an optionally substituted 5-6-membered aryl or heteroaryl ring (e.g., a 5-6 membered aryl or heteroaryl substituted with 0, 1, 2, or 3 substituents selected from hydroxy, halogen, alkoxy, alkyl, haloalkyl, amino, alkylamino and cyano);
W_{PTM3A} is an optionally substituted 5-6-membered aryl or heteroaryl ring (e.g., a 5-6 membered aryl or heteroaryl substituted with 0, 1, 2, or 3 substituents selected from hydroxy, halogen, alkoxy, alkyl, haloalkyl, amino, alkylamino and cyano), or an optionally substituted 3-12 cycloalkyl or heterocycloalkyl (e.g. a 3-12 cycloalkyl or heterocycloalkyl substituted with 0, 1, or 2 substituents selected from hydroxy, halogen, alkoxy, alkyl, haloalkyl, amino, alkylamino and cyano), such as optionally substituted bridged bicycloalkyl and bridged biheterocyclyl rings, optionally substituted 6-11 membered fused bicycloalkyl or fused biheterocycloalkyl rings (e.g. 6-11 membered fused bicycloalkyl or fused biheterocycloalkyl rings with 0, 1, or 2 substituents selected from hydroxy, halogen, alkoxy, alkyl, haloalkyl, amino, alkylamino and cyano), optionally substituted 6-12 membered spirocycloalkyl or spiroheterocyclyl rings (e.g. 6-12 member spirocycloalkyl or spiroheterocyclyl rings substituted with 0, 1, or 2 substituents selected from hydroxy, halogen, alkoxy, alkyl, haloalkyl, amino, alkylamino and cyano);
W_{PTM3} is an optionally substituted 3-9-membered aryl or heteroaryl ring (e.g., an optionally substituted 5-6-membered aryl or heteroaryl ring, or a 3-9 or 5-6 membered aryl or heteroaryl substituted with 0, 1, 2, or 3 substituents selected from hydroxy, halogen, alkoxy, alkyl, haloalkyl, amino, alkylamino and cyano), or an optionally substituted 4-10 membered cycloalkyl or heterocyclyl, such as an optionally substituted bridged bicycloalkyl and bridged biheterocyclyl rings (e.g. a 4-10 membered cycloalkyl or heterocyclyl substituted with 0, 1, or 2 substituents selected from hydroxy, halogen, alkoxy, alkyl, haloalkyl, amino, alkylamino and cyano);
W_{PTM5A} is absent (such that W_{PTM3} is connected directly to L (linker) or ULM), an optionally substituted 5-6-membered aryl or heteroaryl ring (e.g. a 5-6 membered aryl or heteroaryl substituted with 0, 1, or 2 substituents selected from hydroxy, halogen, alkoxy, alkyl, haloalkyl, amino, alkylamino and cyano), or an optionally substitute 3-7 cycloalkyl or heterocycloalkyl (e.g., optionally substituted 5-7 cycloalkyl or heterocycloalkyl, or a 3-7 or 5-7 cycloalkyl or heterocycloalkyl substituted with 0, 1, 2, or 3 substituents selected from hydroxy, halogen, alkoxy, alkyl, haloalkyl, amino, alkylamino and cyano);
W_{PTM5} is absent (such that W_{PTM3} is connected directly to L (linker) or ULM) or an optionally substituted alkyl, an optionally substituted 5-6-membered cycloalkyl, heterocycle, aryl or heteroaryl ring (e.g. a 5-6 membered cycloalkyl, heterocycle, aryl or heteroaryl substituted with 0, 1, or 2 substituents selected from hydroxy, halogen, alkoxy, alkyl, haloalkyl, amino, alkylamino and cyano); and
[symbol] is the attachment point to the, linker, ULM group, ULM′ group, VLM group, VLM′ group,
wherein at least one of the following is present:
   W_{PTM3A} is optionally substituted 6-11 membered fused bicycloalkyl rings, optionally substituted 6-11 membered fused biheterocycloalkyl rings, optionally substituted 6-12 membered spirocycloalkyl, or optionally substituted 6-12 membered spiroheterocyclyl rings (e.g. each optionally substituted with 0, 1, or 2 substituents selected from hydroxy, halogen, alkoxy, alkyl, haloalkyl, amino, alkylamino and cyano); or
   W_{PTM5A} is an optionally substitute 3-7 cycloalkyl or heterocycloalkyl (e.g., optionally substituted 5-7 cycloalkyl or heterocycloalkyl or a 3-7 or 5-7 cycloalkyl or heterocycloalkyl substituted with 0, 1, 2, or 3 substituents selected from hydroxy, halogen, alkoxy, alkyl, haloalkyl, amino, alkylamino and cyano); or
a combination thereof.

In any aspect or embodiment described herein, W_{PTM3A} is optionally substituted 6-11 membered fused bicycloalkyl rings, optionally substituted 6-11 membered fused biheterocycloalkyl rings, optionally substituted 6-12 membered spirocycloalkyl, or optionally substituted 6-12 membered spiroheterocyclyl rings (e.g. each optionally substituted with 0, 1, or 2 substituents selected from hydroxy, halogen, alkoxy, alkyl, haloalkyl, amino, alkylamino and cyano).

In any aspect or embodiment described herein, W_{PTM5} is a piperidine.

In any aspect or embodiment described herein, W_{PTM1} comprises a phosphate substitution.

In any aspect or embodiment described herein, the PTM of the compound of the present disclosure is represented by Formula I, wherein at least one of:
W_{PTM1} is an optionally substituted phenyl or a pyridyl (e.g., substituted as described herein, such as a phenyl substituted with a hydroxy or phosphate substituent with or without an additional optional substituent selected as described herein, e.g., substituted with 0, 1, 2, or 3 substituents selected from hydroxy, halogen, alkoxy, alkyl, haloalkyl, amino, alkylamino, cyano or combination thereof);
W_{PTM2} is an optionally substituted 6-membered heteroaryl ring (e.g., substituted as described herein, such as a pyridazine substituted with amino group);
W_{PTM3} is an optionally substituted 5-6-membered heteroaryl (e.g., a pyrazole, pyrrole, imidazole, oxazole, oxadiazole, or triazole);
W_{PTM3A} is optionally substituted 6-11 membered fused bicycloalkyl rings, optionally substituted 6-11 membered fused biheterocycloalkyl rings, optionally substituted 6-12 membered spirocycloalkyl, or optionally substituted 6-12 membered spiroheterocyclyl rings (e.g. each optionally substituted with 0, 1, or 2 substituents selected from hydroxy, halogen, alkoxy, alkyl, haloalkyl, amino, alkylamino and cyano);
W_{PTM5} is as described in any aspect or embodiment described herein (e.g., W_{PTM5} may be absent or a pyridine ring);
W_{PTM5A} is an optionally substitute 3-7 cycloalkyl or heterocycloalkyl (e.g., optionally substituted 5-7 cycloalkyl or heterocycloalkyl or a 3-7 or 5-7 cycloalkyl or heterocycloalkyl substituted with 0, 1, 2, or 3 substituents selected from hydroxy, halogen, alkoxy, alkyl, haloalkyl, amino, alkylamino and cyano); or
a combination thereof.

In any aspect or embodiment described herein, for example, an embodiment that includes a PTM of Formula I, W_{PTM3} is a pyrazole or a 6-8-membered heterocyclyl (e.g., a piperazine or a diazabicyclooctane).

In any aspect or embodiment described herein, the PTM of the present disclosure is represented by:

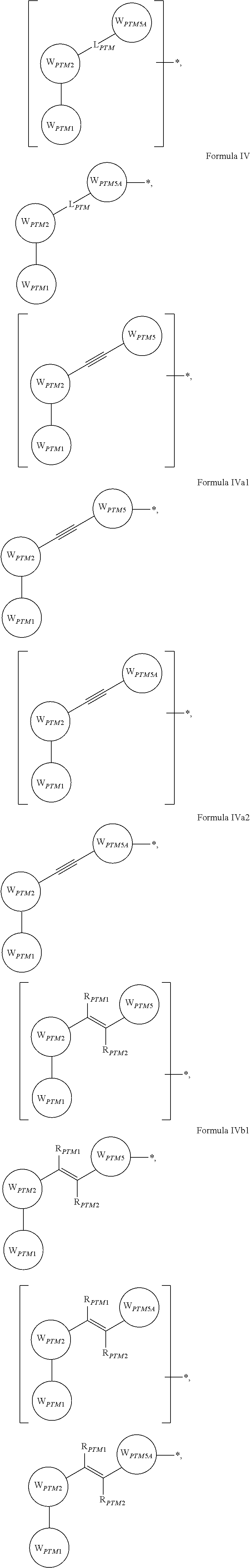

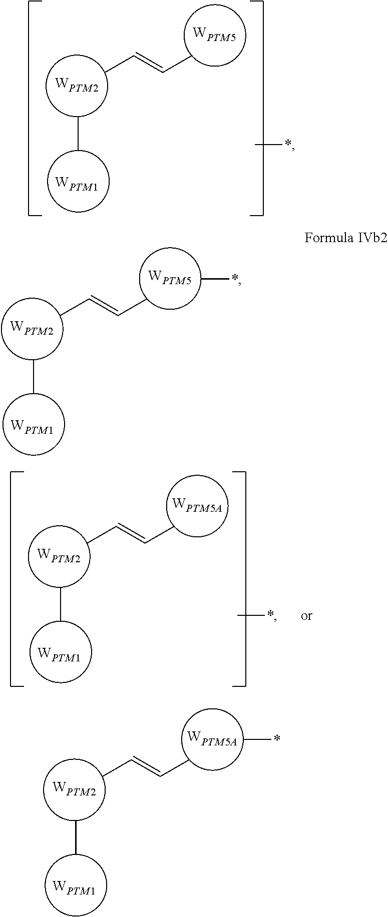

wherein:
W_{PTM1} is as described in any other aspect or embodiment described herein, such as W_{PTM1} is an optionally substituted 5-6-membered aryl or heteroaryl ring (e.g., a 5-6 member aryl or heteroaryl substituted with 0, 1, 2, or 3 substituents selected from hydroxy, halogen, alkoxy, alkyl, haloalkyl, amino, alkylamino and cyano);
W_{PTM2} is as described in any other aspect or embodiment described herein, such as W_{PTM2} is an optionally substituted 5-6-membered aryl or heteroaryl ring (e.g., a 5-6 membered aryl or heteroaryl substituted with 0, 1, 2, or 3 substituents selected from hydroxy, halogen, alkoxy, alkyl, haloalkyl, amino, alkylamino and cyano);
W_{PTM5} is as described in any other aspect or embodiment described herein, such as W_{PTM5} is absent (such that L_{PTM} is connected directly to L (linker) or ULM) or an optionally substituted alkyl, an optionally substituted 5-6-membered cycloalkyl, heterocycle, aryl or heteroaryl ring (e.g. a 5-6 membered cycloalkyl, heterocycle, aryl or heteroaryl substituted with 0, 1, or 2 substituents selected from hydroxy, halogen, alkoxy, alkyl, haloalkyl, amino, alkylamino and cyano) with the proviso that a heteroatom is not directly connected to the carbon atom of the carbon-carbon double bond, or a carbon-carbon triple bond;
W_{PTM5A} is as described in any other aspect or embodiment described herein, such as W_{PTM5A} is absent (such that L_{PTM} is connected directly to L (linker) or ULM), an optionally substituted 5-6-membered aryl or heteroaryl ring (e.g. a 5-6 membered aryl or heteroaryl substituted with 0, 1, or 2 substituents selected from hydroxy, halogen, alkoxy, alkyl, haloalkyl, amino, alkylamino and cyano), or an optionally substituted 3-7 cycloalkyl or heterocycloalkyl (e.g., optionally substituted 3-6 cycloalkyl or heterocycloalkyl, or a 3-7 or 3-6 cycloalkyl or heterocycloalkyl substituted with 0, 1, 2, or 3 substituents selected from hydroxy, halogen, alkoxy, alkyl, haloalkyl, amino, alkylamino and cyano) with the proviso that a heteroatom is not directly connected to the carbon atom of the carbon-carbon double bond, or a carbon-carbon triple bond;
L_{PTM} is selected from the group consisting of: a C₂₋₄ alkyne (e.g., a C₂₋₃ or C₂ alkyne) or a C₂₋₄ alkene (e.g., a C₂₋₃ or C₂ alkene) optionally substituted with 1-2 substituents independently selected from methyl, fluoro or haloalkyl; a C₁-C₂ alkyl optionally substituted with 1-2 substituents selected from methyl, fluoro, or haloalkyl; or a cyclopropyl optionally substituted with 1-2 substituents selected from methyl, fluoro, or haloalkyl;
R^{PTM1} and R^{PTM2} are individually a H, halogen, OH, C1-C3 alkyl, C1-C3 haloalky, or C1-C3 alkoxy; and
[symbol] is the attachment point to the, linker, ULM group, ULM′ group, VLM group, VLM′ group.

In any aspect or embodiment described herein, the PTM of the present disclosure has the chemical structure represented by Formula IV, wherein at least one of:
W_{PTM1} is a phenyl substituted with a hydroxy or phosphate substituent with or without an additional optional substituent selected as described herein;
W_{PTM2} is a pyridazine substituted with amino group;
W_{PTM5A} is an optionally substitute 3-7 cycloalkyl or heterocycloalkyl (e.g., optionally substituted 3-6 cycloalkyl or heterocycloalkyl, or a 3-7 or 3-6 cycloalkyl or heterocycloalkyl substituted with 0, 1, 2, or 3 substituents selected from hydroxy, halogen, alkoxy, alkyl, haloalkyl, amino, alkylamino and cyano); or
a combination thereof.

In any aspect or embodiment described herein, the PTM of the present disclosure is represented by:

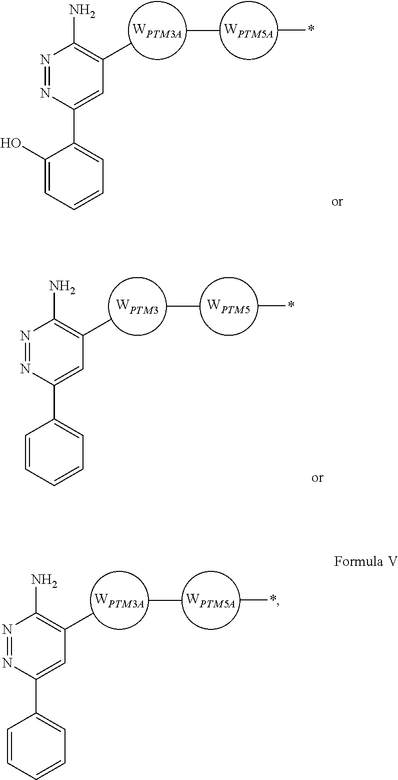

or a pharmaceutically acceptable salt thereof, wherein at least one of the following is present:
W_{PTM3A} is optionally substituted 6-11 membered fused bicycloalkyl rings, optionally substituted 6-11 membered fused biheterocycloalkyl rings, optionally substituted 6-12 membered spirocycloalkyl, or optionally substituted 6-12 membered spiroheterocyclyl rings (e.g. each optionally substituted with 0, 1, or 2 substituents selected from hydroxy, halogen, alkoxy, alkyl, haloalkyl, amino, alkylamino and cyano); or
W_{PTM5A} is an optionally substitute 3-7 cycloalkyl or heterocycloalkyl (e.g., optionally substituted 5-7 cycloalkyl or heterocycloalkyl or a 3-7 or 5-7 cycloalkyl or heterocycloalkyl substituted with 0, 1, 2, or 3 substituents selected from hydroxy, halogen, alkoxy, alkyl, haloalkyl, amino, alkylamino and cyano); or
a combination thereof.

In any aspect or embodiment described herein, the PTM of the present disclosure is represented by:

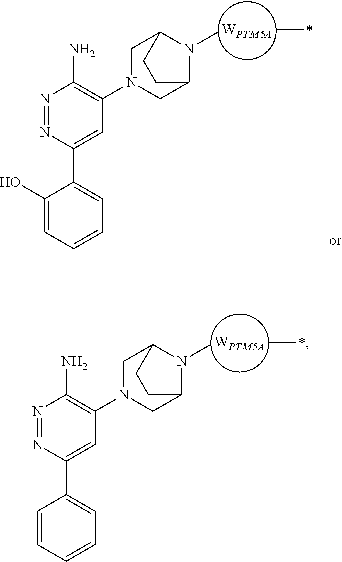

or a pharmaceutically acceptable salt thereof, wherein:
W_{PTM5A} is an optionally substitute 3-7 cycloalkyl or heterocycloalkyl (e.g., optionally substituted 5-7 cycloalkyl or heterocycloalkyl or a 3-7 or 5-7 cycloalkyl or heterocycloalkyl substituted with 0, 1, 2, or 3 substituents selected from hydroxy, halogen, alkoxy, alkyl, haloalkyl, amino, alkylamino and cyano).

In any aspect or embodiment described herein, the PTM of the present disclosure is represented by:

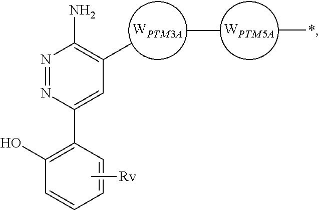

### Formula Vb

or a pharmaceutically acceptable salt thereof,
wherein:
W_{PTM3A} is an optionally substituted 5-6-membered heteroaryl, an optionally substituted 4-9 cycloalkyl or heterocyclyl ring, an optionally substituted bridged bicycloalkyl and bridged biheterocyclyl ring, or as defined in any other aspect or embodiment described herein;
W_{PTM5A} is an optionally substituted 5-6-membered heteroaryl or aryl, e.g., pyridine, or pyridazine, or as defined in any other aspect or embodiment described herein;
Rv is 0, 1, 2 or 3 substituents independently selected from hydroxy, halogen, alkoxy, alkyl, haloalkyl, phosphate, amino, alkylamino, cyano, or a combination thereof;
at least one of the following is present:
W_{PTM3A} is optionally substituted 6-11 membered fused bicycloalkyl rings, optionally substituted 6-11 membered fused biheterocycloalkyl rings, optionally substituted 6-12 membered spirocycloalkyl, or optionally substituted 6-12 membered spiroheterocyclyl rings (e.g. each optionally substituted with 0, 1, or 2 substituents selected from hydroxy, halogen, alkoxy, alkyl, haloalkyl, amino, alkylamino and cyano); or
W_{PTM5A} is an optionally substitute 3-7 cycloalkyl or heterocycloalkyl (e.g., optionally substituted 5-7 cycloalkyl or heterocycloalkyl or a 3-7 or 5-7 cycloalkyl or heterocycloalkyl substituted with 0, 1, 2, or 3 substituents selected from hydroxy, halogen, alkoxy, alkyl, haloalkyl, amino, alkylamino and cyano); or
a combination thereof.

In certain embodiments, the hydroxyl group is modified with a phosphate group (i.e., a phosphoester group).

In any aspect or embodiment described herein, the PTM of the present disclosure has a chemical structure represented by:

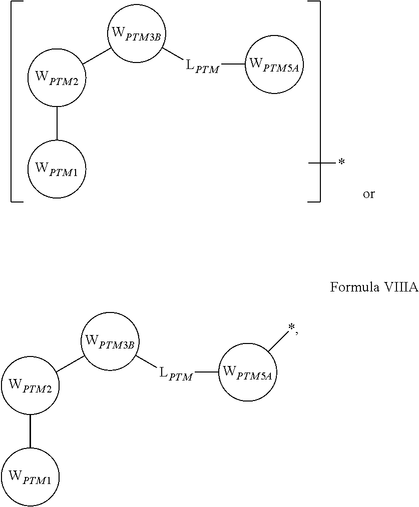

wherein:
W_{PTM1} is an optionally substituted 5-6-membered aryl or heteroaryl ring (e.g., a 5-6 member aryl or heteroaryl substituted with 0, 1, 2, or 3 substituents selected from hydroxy, halogen, alkoxy, alkyl, haloalkyl, amino, alkylamino and cyano);
W_{PTM2} is an optionally substituted 5-6-membered aryl or heteroaryl ring (e.g., a 5-6 membered aryl or heteroaryl substituted with 0, 1, 2, or 3 substituents selected from hydroxy, halogen, alkoxy, alkyl, haloalkyl, amino, alkylamino and cyano);
W_{PTM3B} is an optionally substituted 5-6-membered aryl or heteroaryl ring (e.g., a 5-6 membered aryl or heteroaryl substituted with 0, 1, 2, or 3 substituents selected from hydroxy, halogen, alkoxy, alkyl, haloalkyl, amino, alkylamino and cyano), an optionally substituted 3-12 cycloalkyl or heterocycloalkyl (e.g. a 3-12 cycloalkyl or heterocycloalkyl substituted with 0, 1, or 2 substituents selected from hydroxy, halogen, alkoxy, alkyl, haloalkyl, amino, alkylamino and cyano), optionally substituted 3-10 membered fused bicycloalkyl or fused biheterocyclyl rings (e.g., 3-10 membered fused bicycloalkyl or fused biheterocyclyl rings substituted with 0, 1, or 2 substituents selected from hydroxy, halogen, alkoxy, alkyl, haloalkyl, amino, alkylamino and cyano), optionally substituted 3-12 membered bridged bicycloalkyl or bridged biheterocyclyl rings (e.g. a 3-12 membered bridged bicycloalkyl or bridged biheterocyclyl rings substituted with 0, 1, or 2 substituents selected from hydroxy, halogen, alkoxy, alkyl, haloalkyl, amino, alkylamino and cyano), an optionally substituted 6-12 membered spirocycloalkyl or spiroheterocyclyl rings (e.g. a 6-12 member spirocycloalkyl or spiroheterocyclyl rings substituted with 0, 1, or 2 substituents selected from hydroxy, halogen, alkoxy, alkyl, haloalkyl, amino, alkylamino and cyano);
W_{PTM5A} is an optionally substituted 5-6-membered aryl or heteroaryl ring (e.g. a 5-6 membered aryl or heteroaryl substituted with 0, 1, or 2 substituents selected from hydroxy, halogen, alkoxy, alkyl, haloalkyl, amino, alkylamino and cyano), or an optionally substitute 3-7 cycloalkyl or heterocyclyl (e.g., optionally substituted 5-7 cycloalkyl or heterocycloalkyl, or a 3-7 or 5-7 cycloalkyl or heterocycloalkyl substituted with 0, 1, 2, or 3 substituents selected from hydroxy, halogen, alkoxy, alkyl, haloalkyl, amino, alkylamino and cyano);
L_{PTM} is O or C1-C2 alkyl (e.g., a methylene group) optionally substituted with 0, 1, 2, or 3 groups selected from an C1-C4 alkyl (e.g., methyl or ethyl), C1-C3 alkoxy (e.g. methoxy or ethoxy), halogen (e.g., F or Cl), and =O; and
[symbol] is the attachment point to the, linker, ULM group, ULM′ group, VLM group, VLM′ group,
wherein at least one of the following is present:
   W_{PTM3B} is optionally substituted 3-10 fused bicycloalkyl rings, optionally substituted 3-10 fused biheterocyclyl rings, optionally substituted 6-12 membered spirocycloalkyl rings, or optionally substituted 6-12 membered spiroheterocyclyl rings (e.g. each is optionally substituted with 0, 1, or 2 substituents selected from hydroxy, halogen, alkoxy, alkyl, haloalkyl, amino, alkylamino and cyano); or
   W_{PTM5A} is an optionally substituted 5-6-membered aryl or heteroaryl ring (e.g. a 5-6 membered aryl or heteroaryl substituted with 0, 1, or 2 substituents selected from hydroxy, halogen, alkoxy, alkyl, haloalkyl, amino, alkylamino and cyano); or
   a combination thereof.

In any aspect or embodiment described herein, wherein the PTM has a chemical structure of Formula IA, IV, or VIIIA, wherein at least one of:
W_{PTM3A} is an optionally substituted 6-12 membered spirocycloalkyl or spiroheterocyclyl rings (e.g. a 6-12 member spirocycloalkyl or spiroheterocyclyl rings substituted with 0, 1, or 2 substituents selected from hydroxy, halogen, alkoxy, alkyl, haloalkyl, amino, alkylamino and cyano);
W_{PTM3B} is an optionally substituted 3-10 fused bicycloalkyl or fused biheterocyclyl rings (e.g. a 3-10 fused bicycloalkyl or fused biheterocyclyl rings substituted with 0, 1, or 2 substituents selected from hydroxy, halogen, alkoxy, alkyl, haloalkyl, amino, alkylamino and cyano), or an optionally substituted 6-12 membered spirocycloalkyl or spiroheterocyclyl rings (e.g. a 6-12 member spirocycloalkyl or spiroheterocyclyl rings substituted with 0, 1, or 2 substituents selected from hydroxy, halogen, alkoxy, alkyl, haloalkyl, amino, alkylamino and cyano); and
W_{PTM5A} is an optionally substitute 3-7 cycloalkyl or heterocycloalkyl (e.g., optionally substituted 5-7 cycloalkyl or heterocycloalkyl, or a 3-7 or 5-7 cycloalkyl or heterocycloalkyl substituted with 0, 1, 2, or 3 substituents selected from hydroxy, halogen, alkoxy, alkyl, haloalkyl, amino, alkylamino and cyano).

In any aspect or embodiment described herein, the PTM has the chemical structure:

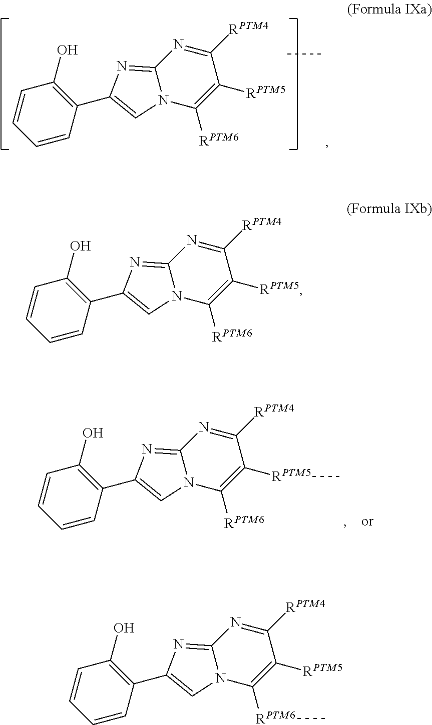

wherein:
the [symbol] of Formula IXa is the attachment point to the linker, ULM group, ULM′ group, VLM group, VLM′ group; and
the attachment point to the linker, ULM group, ULM′ group, VLM group, VLM′ group in Formula IXb is to the R^{PTM5} substitution or the R^{PTM6} substitution (e.g., the R^{PTM5} or the R^{PTM6} that is substituted) or at that location the R^{PTM5} location or the R^{PTM6} locations, such that the R^{PTM5} or the R^{PTM6} is a bond
R^{PTM4} is selected from H, —NH₂, —OH, or —NR^{PTM7}R^{PTM8}, wherein R^{PTM7} and R^{PTM8} are independently selected C₁₋₄ alkyls;
one of R^{PTM5} and R^{PTM6} is H or a bond when the point of attachment, and the other is selected from a H, a bond when the point of attachment, 5- or 6-membered aryl, 5- or 6-membered heteroaryl (e.g.,

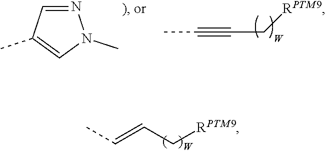

wherein W is 0, 1, or 2;
R^{PTM9} is selected from a H, alkyl (e.g., methyl), methoxy, optionally substituted 5- or 6-membered aryl, optionally substituted 5- or 6-membered heteroaryl (e.g.,

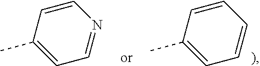

optionally substituted 4-6 membered cycloalkyl (e.g., optionally substituted 4- or 6-membered cycloalkyl), optionally substituted 4-6 heterocycloalkyl (e.g., optionally substituted 4- or 6-membered heterocycloalkyl), optionally substituted —O-4-6 membered cycloalkyl (e.g., —O-optionally substituted 4- or 6-membered cycloalkyl), optionally substituted —O-4-6 heterocycloalkyl (e.g., —O-optionally substituted 4- or 6-membered heterocycloalkyl), or C₁₋₆ alkylene group optionally interspaced with 0 atoms, provided that no heteroatom is directly attached to the double or triple carbon-carbon bond and any two heteroatoms are separated by at least two carbon atoms.

In any aspect or embodiment described herein, optional substitution of R^{PTM9} is one or more (1, 2, 3, or 4) substituents independently selected from C₁₋₆ alkyl, C₁₋₆ haloalkyl, halogen, cyano, and methoxy.

In any aspect or embodiment described herein, R^{PTM4} is selected from H, —NH₂, or —OH, or —NR^{PTM7}R^{PTM8}, wherein R^{PTM7} and R^{PTM8} are independently selected C₁-C₄ alkyls.

In any aspect or embodiment described herein, R^{PTM4} is selected from H, —NH₂, or —OH.

In any aspect or embodiment described herein, one of R^{PTM5} and R^{PTM6} is H or a bond when the point of attachment, and the other is selected from a H, a bond when the point of attachment 5- or 6-membered aryl, 5- or 6-membered heteroaryl (e.g.,

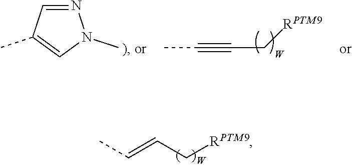

wherein W is 0, 1, or 2, and R^{PTM9} is selected from a H, alkyl (e.g., methyl), methoxy, optionally substituted 4- or 6-membered cycloalkyl, optionally substituted 4- or 6-membered heterocycloalkyl, optionally substituted —O-4- or 6-membered cycloalkyl, optionally substituted —O-4- or 6-membered heterocycloalkyl, optionally substituted 5- or 6-membered aryl, optionally substituted 5- or 6-membered heteroaryl (e.g.,

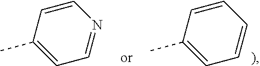

or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from a H, 5- or 6-membered heteroaryl (e.g.,

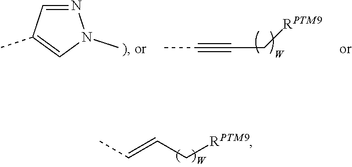

wherein W is 0, 1, or 2, and R^{PTM9} is selected from a H, alkyl (e.g., methyl), methoxy, optionally substituted 4- or 6-membered cycloalkyl, optionally substituted 4- or 6-membered heterocycloalkyl, optionally substituted —O-4- or 6-membered cycloalkyl, optionally substituted —O-4- or 6-membered heterocycloalkyl, optionally substituted 5- or 6-membered heteroaryl (e.g.,

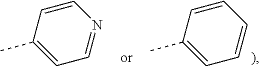

or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from a H, 5-membered heteroaryl (e.g.,

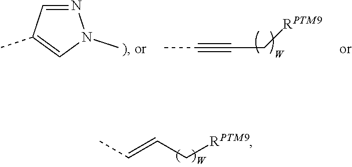

wherein W is 0, 1, or 2, and R^{PTM9} is selected from a H, alkyl (e.g., methyl), methoxy, optionally substituted 4- or 6-membered cycloalkyl, optionally substituted 4- or 6-membered heterocycloalkyl, optionally substituted —O-4- or 6-membered cycloalkyl, optionally substituted —O-4- or 6-membered heterocycloalkyl, optionally substituted 5- or 6-membered heteroaryl (e.g.,

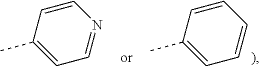

or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from a H, 5-membered heteroaryl (e.g.,

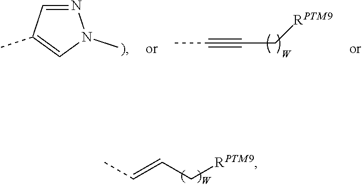

wherein W is 0, 1, or 2, and R^{PTM9} is selected from a H, alkyl (e.g., methyl), methoxy, optionally substituted 4- or 6-membered cycloalkyl, optionally substituted 4- or 6-membered heterocycloalkyl, optionally substituted —O-4- or 6-membered cycloalkyl, optionally substituted —O-4- or 6-membered heterocycloalkyl, optionally substituted 5- or 6-membered heteroaryl (e.g.,

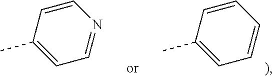

or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from a H, 5-membered heteroaryl (e.g.,

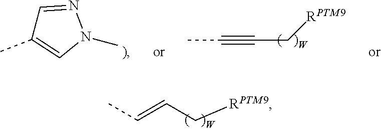

wherein W is 0, 1, or 2, and R^{PTM9} is selected from a H, alkyl (e.g., methyl), methoxy, optionally substituted 4- or 6-membered cycloalkyl, optionally substituted 4- or 6-membered heterocycloalkyl, optionally substituted —O-4- or 6-membered cycloalkyl, optionally substituted —O-4- or 6-membered heterocycloalkyl, optionally substituted 5- or 6-membered heteroaryl (e.g.,

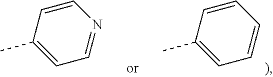

or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H or a bond when the point of attachment, and the other is selected from a H, a bond when the point of attachment, 5- or 6-membered aryl, 5- or 6-membered heteroaryl (e.g.,

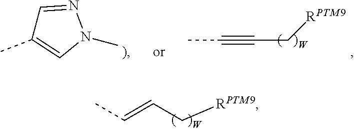

wherein W is 0, 1, or 2, and R^{PTM9} is selected from a H, alkyl (e.g., methyl), methoxy, or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H or a bond when the point of attachment, and the other is selected from a H, a bond when the point of attachment 5- or 6-membered aryl, 5- or 6-membered heteroaryl (e.g.,

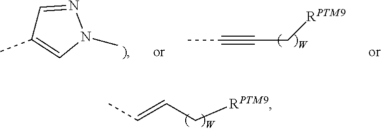

wherein W is 0, 1, or 2, and R^{PTM9} is selected from a H, alkyl (e.g., methyl), methoxy, or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from a H, 5- or 6-membered heteroaryl (e.g.,

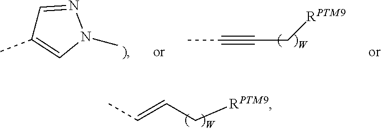

wherein W is 0, 1, or 2, and R^{PTM9} is selected from a H, alkyl (e.g., methyl), methoxy, or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from a H, 5-membered heteroaryl (e.g.,

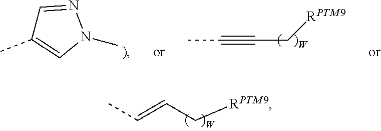

wherein W is 0, 1, or 2, and R^{PTM9} is selected from a H, alkyl (e.g., methyl), methoxy, or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from a H, 5-membered heteroaryl (e.g.,

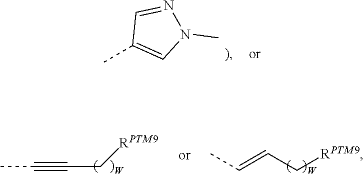

wherein W is 0, 1, or 2, and R^{PTM9} is selected from a H, alkyl (e.g., methyl), methoxy, or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from a H, 5-membered heteroaryl (e.g,

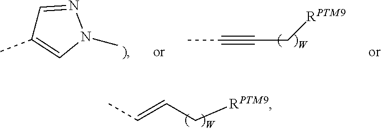

wherein W is 0, 1, or 2, and R^{PTM9} is selected from a H, alkyl (e.g., methyl), methoxy, or C₁₋₆ alkylene group optionally interspaced with O atoms, provided that no heteroatom is directly attached to the double or triple carbon-carbon bond and any two heteroatoms are separated by at least two carbon atoms.

In any aspect or embodiment described herein, one of R^{PTM5} and R^{PTM6} is H, and the other is selected from H,

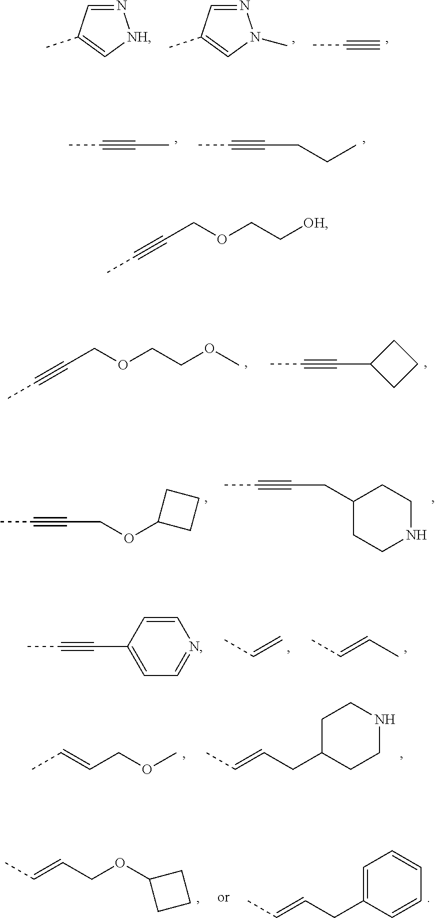

In any aspect or embodiment described herein, one of R^{PTM5} and R^{PTM6} is H, and the other is selected from H,

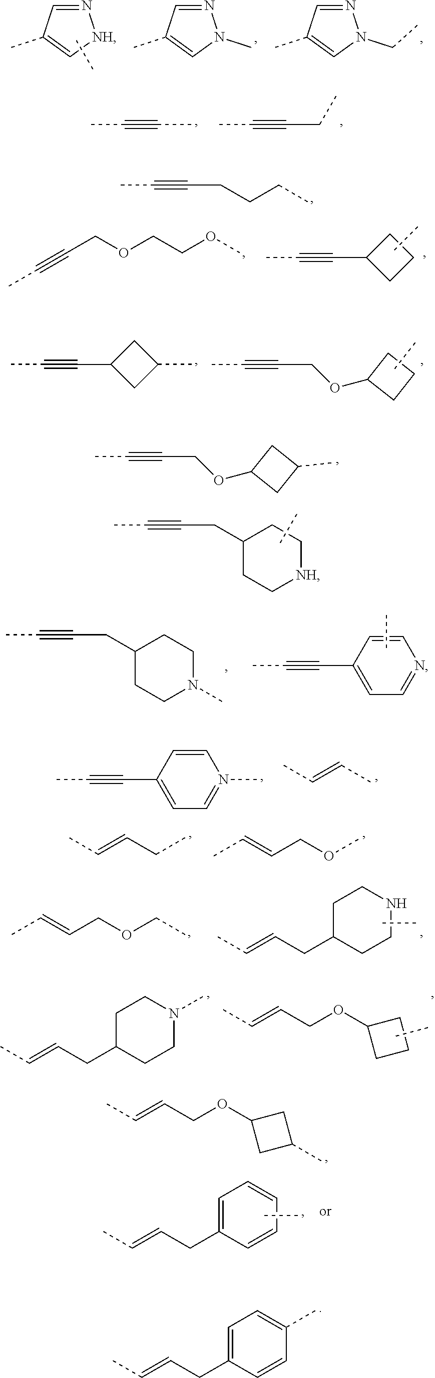

In any aspect or embodiment described herein, one of R^{PTM5} and R^{PTM6} is H, and the other is selected from H,

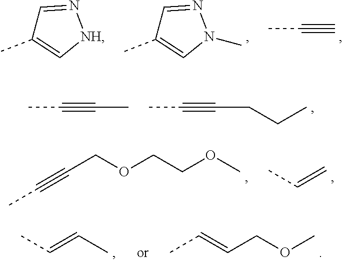

In any aspect or embodiment described herein, one of R^{PTM5} and R^{PTM6} is H, and the other is selected from H,

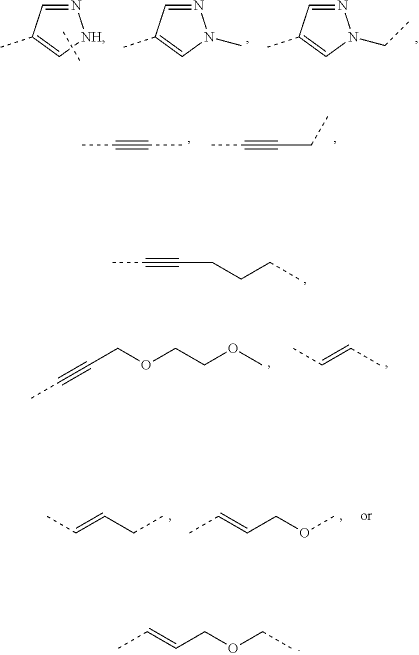

In any aspect or embodiment described herein, the PTM is selected from:

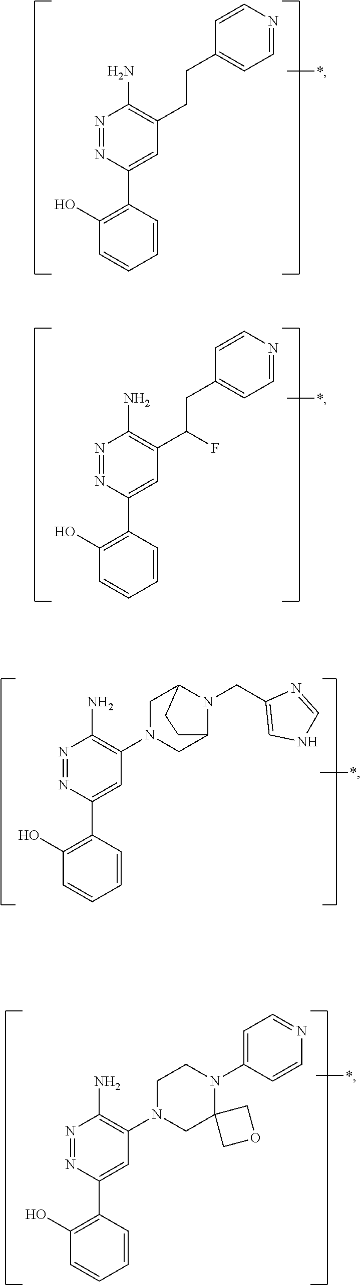

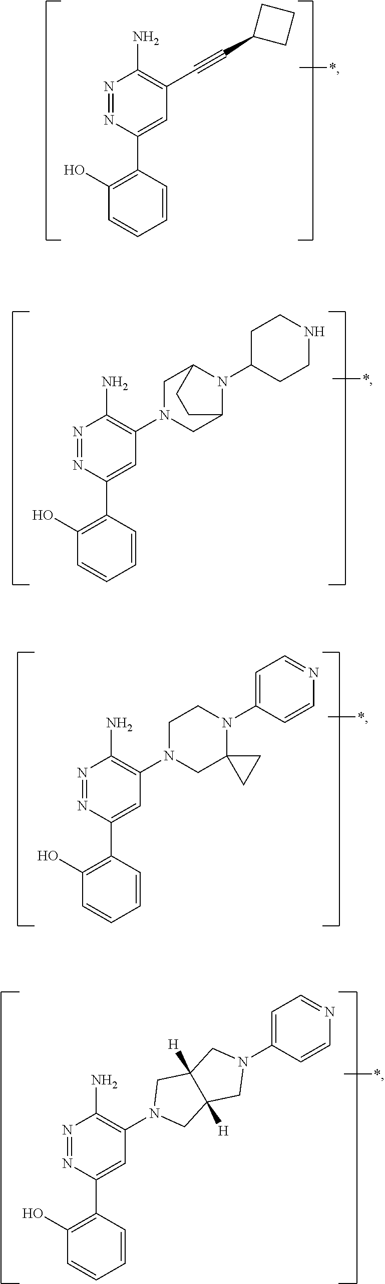

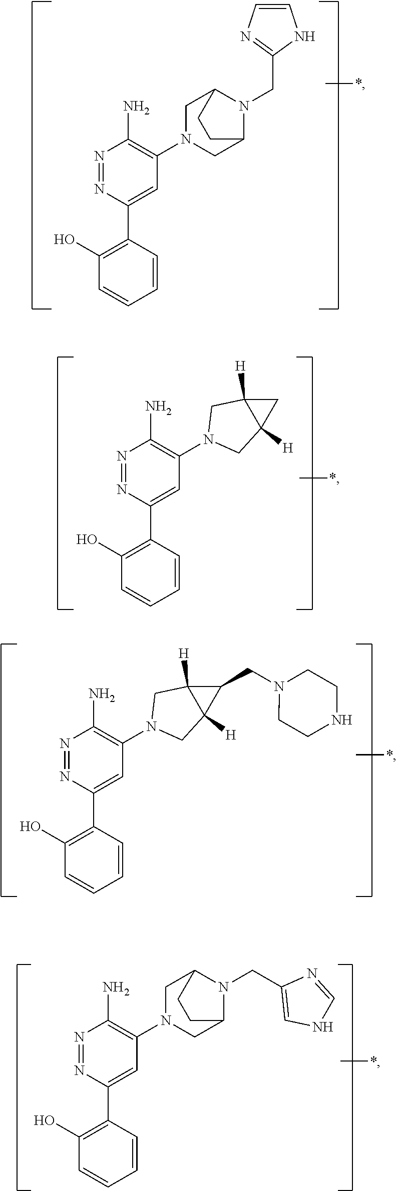

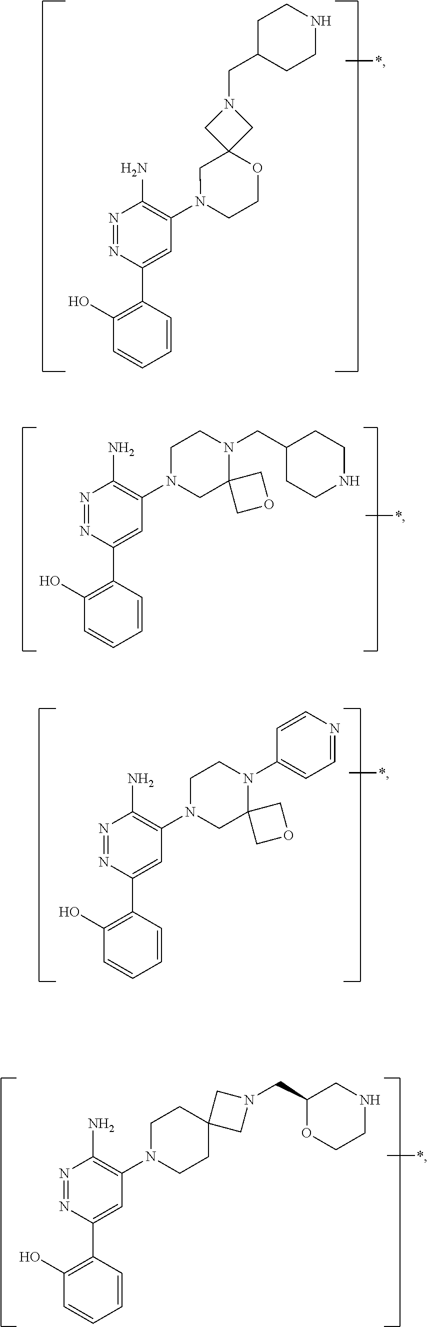

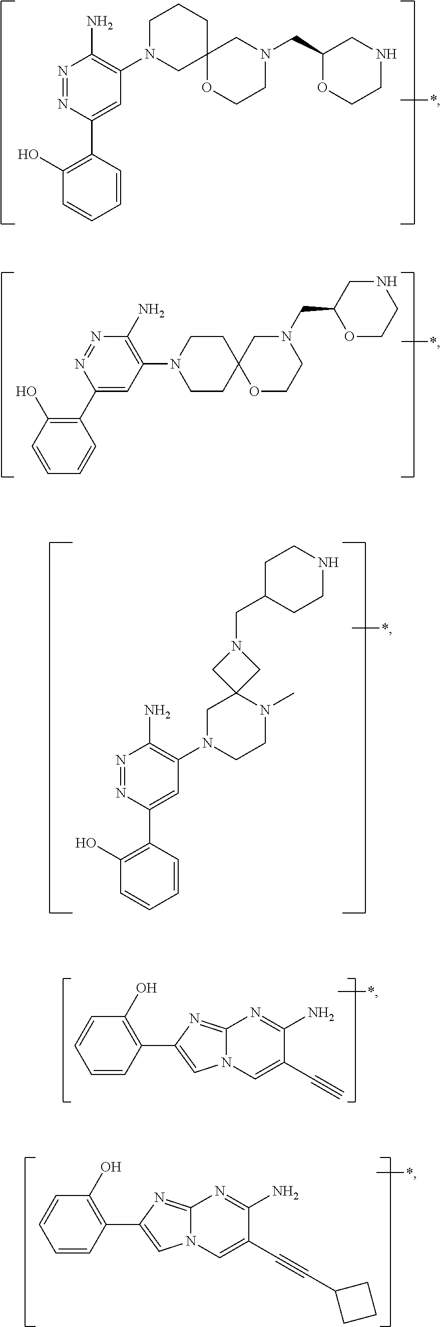

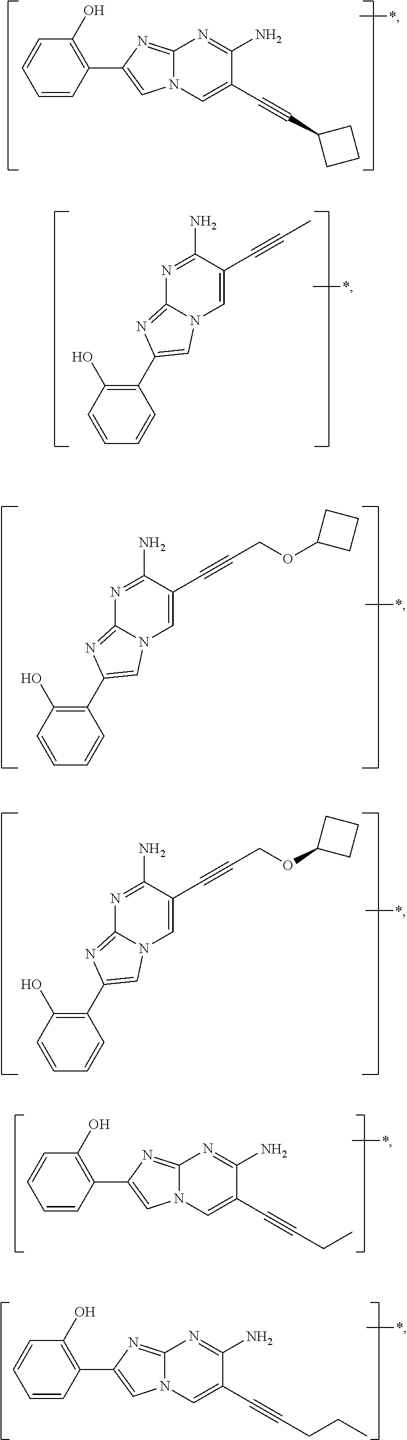

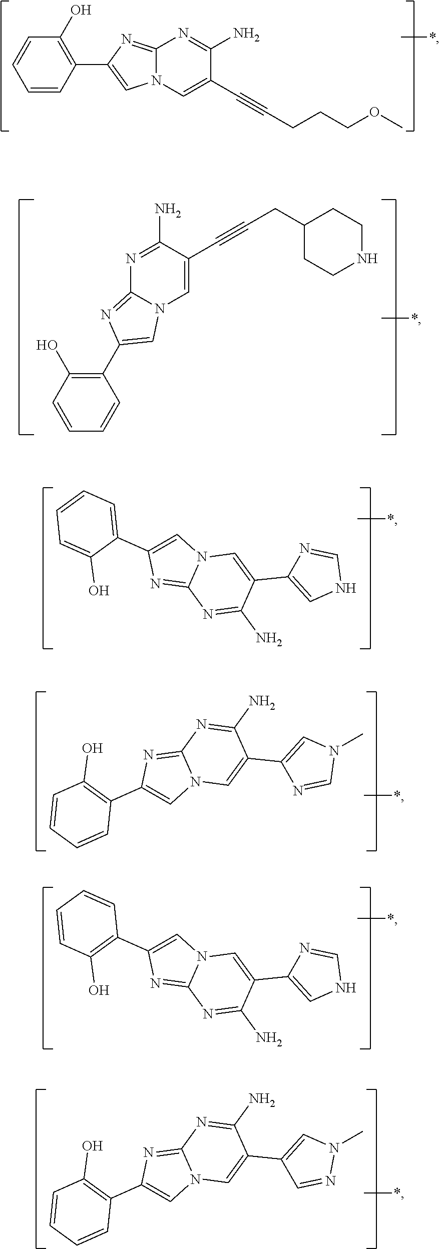

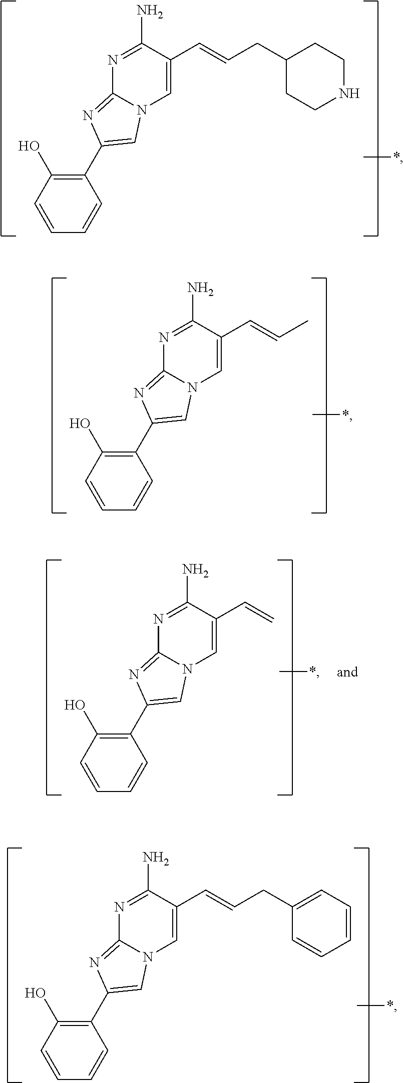

wherein [symbol] is the attachment point to the chemical linking moiety (L).

In any aspect or embodiment described herein, the PTM is selected from:

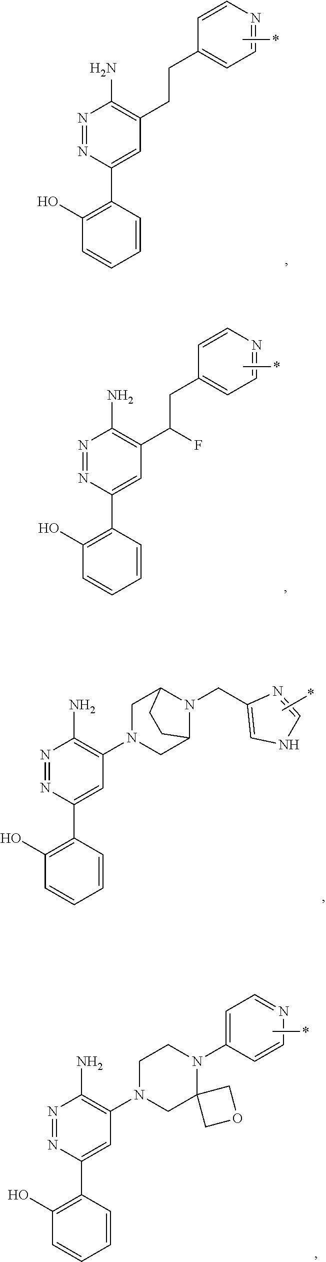

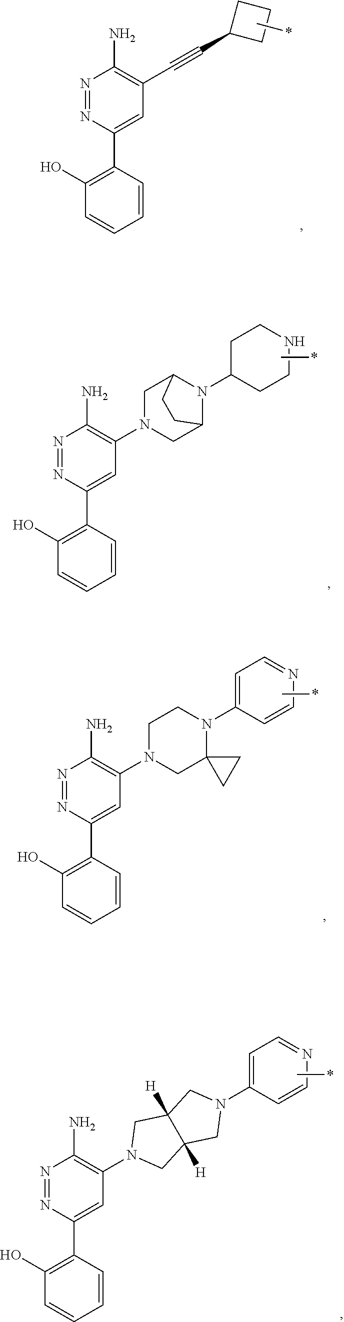

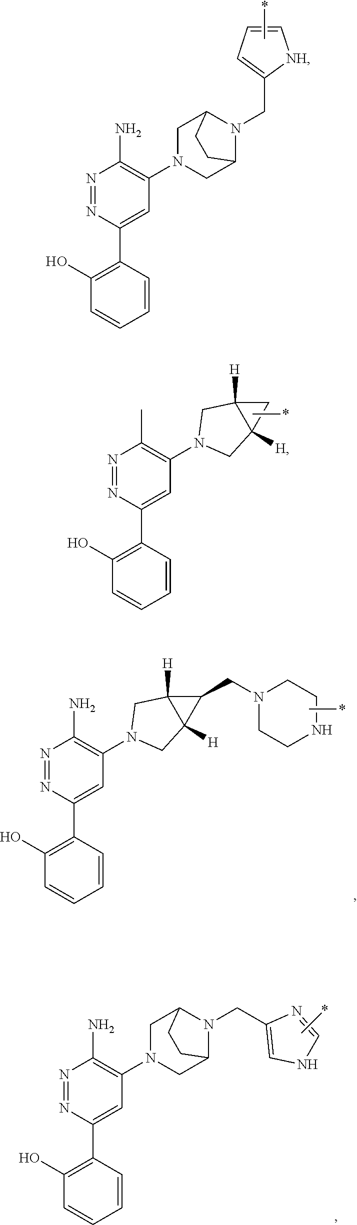

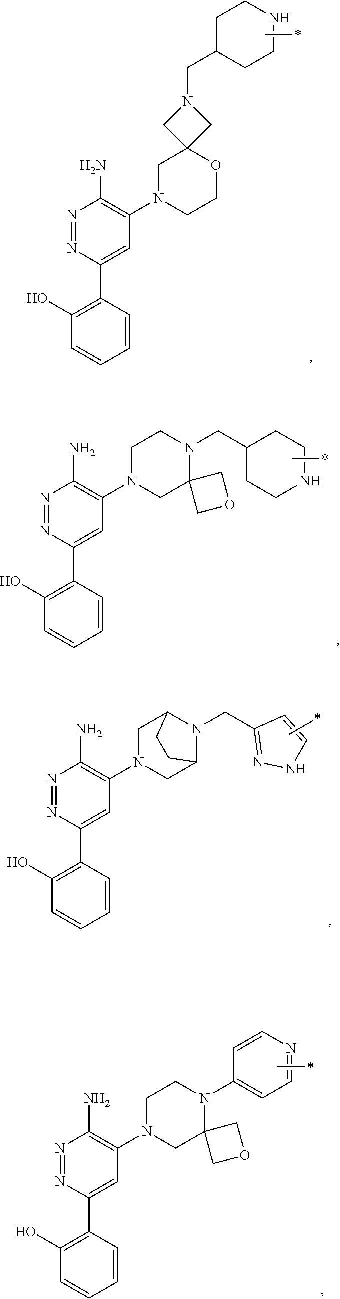

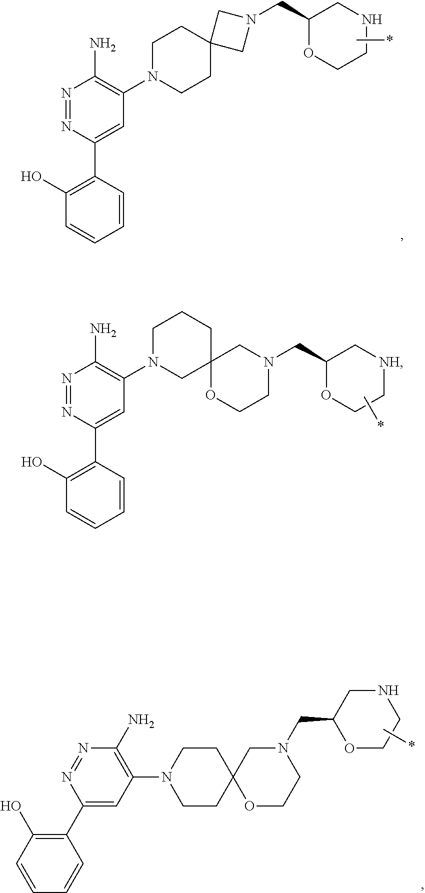

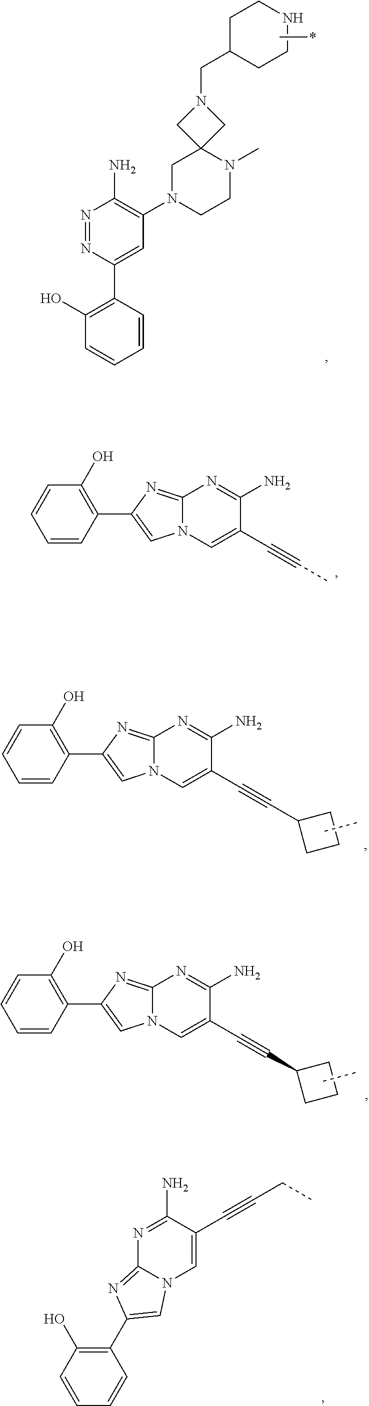

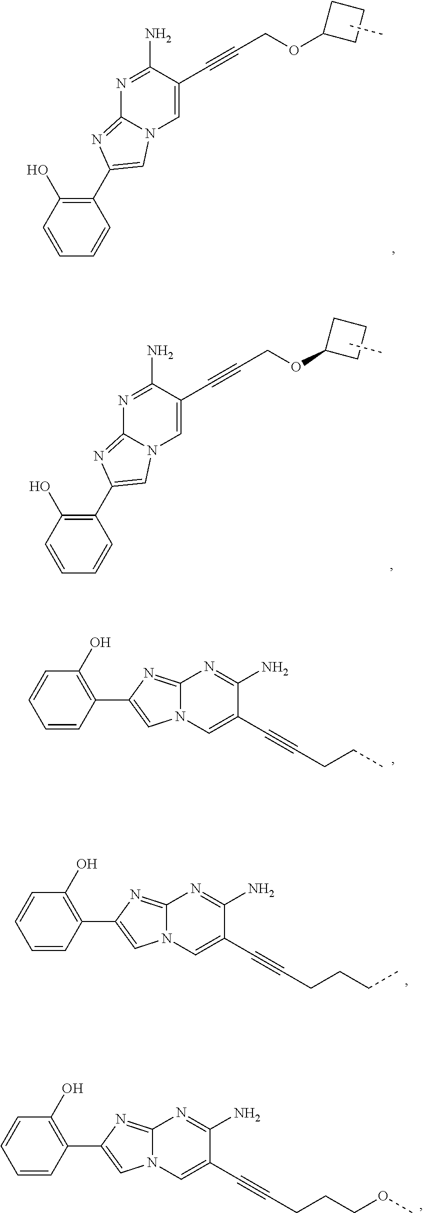

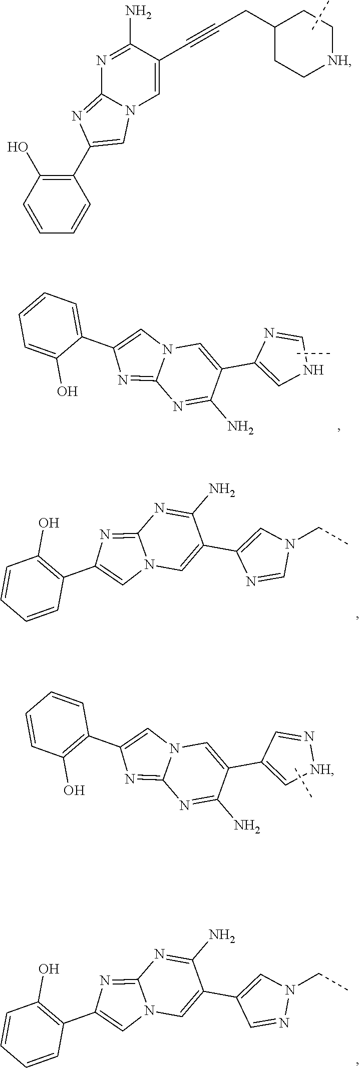

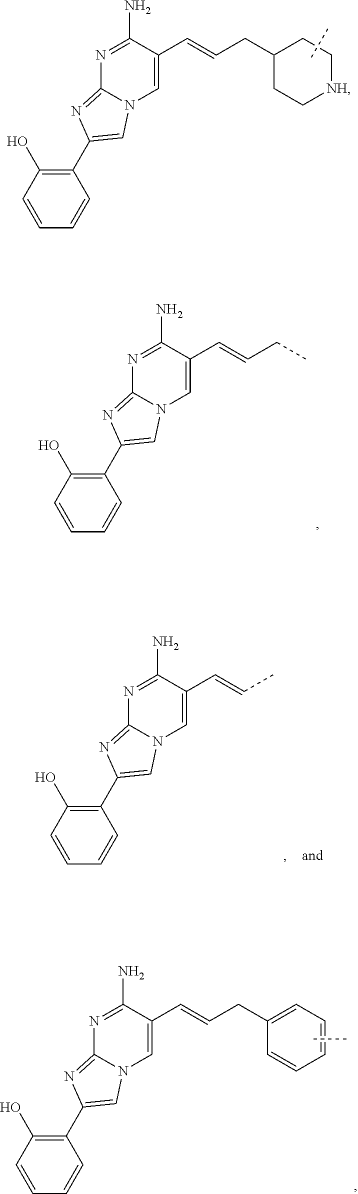

wherein [symbol] is the attachment point to the chemical linking moiety (L) (e.g., the chemical linking moiety is attached to a carbon of the indicated ring or a nitrogen of the indicated ring).

In any aspect or embodiment described herein, the PTM is selected from:

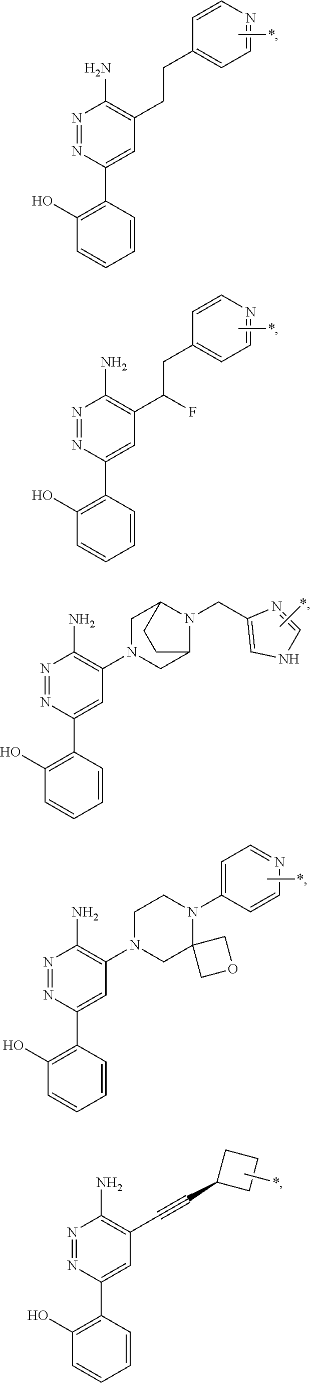

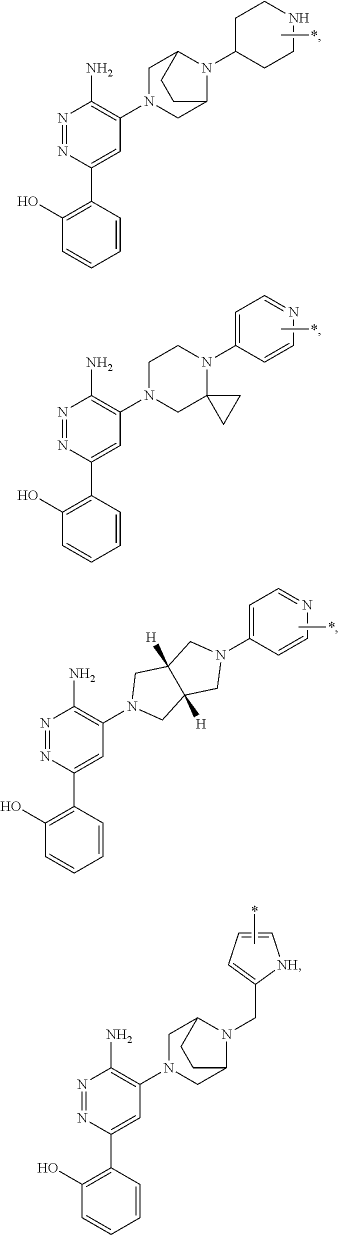

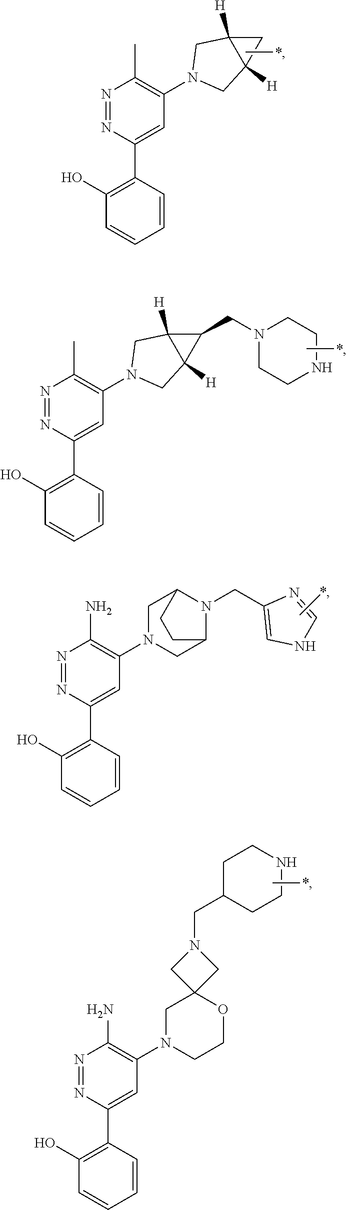

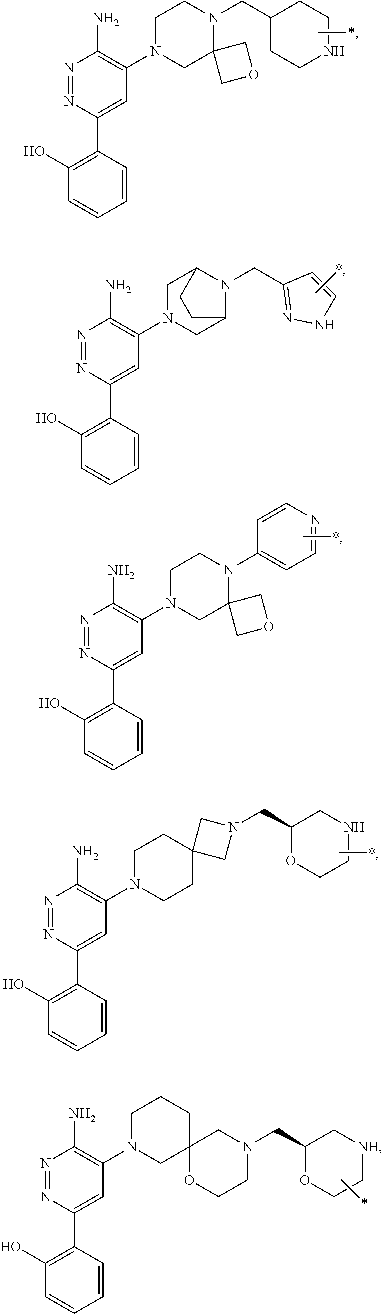

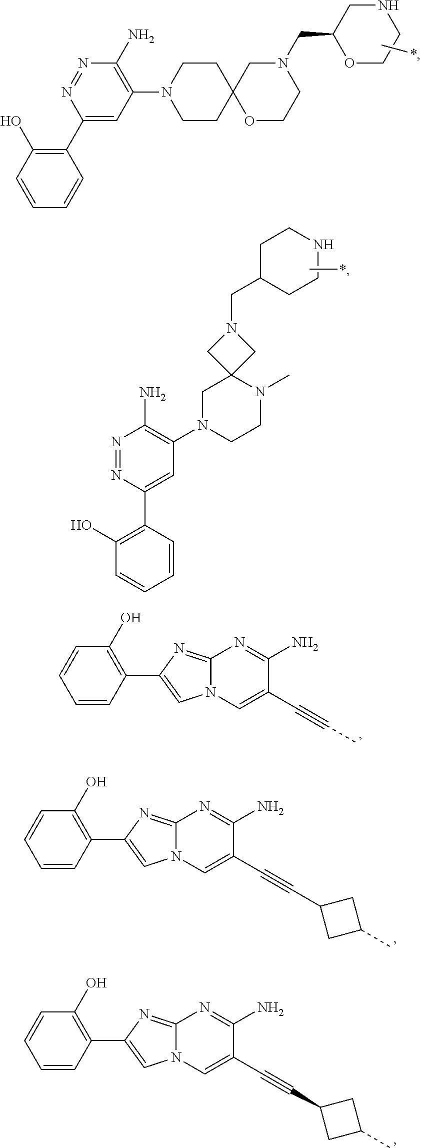

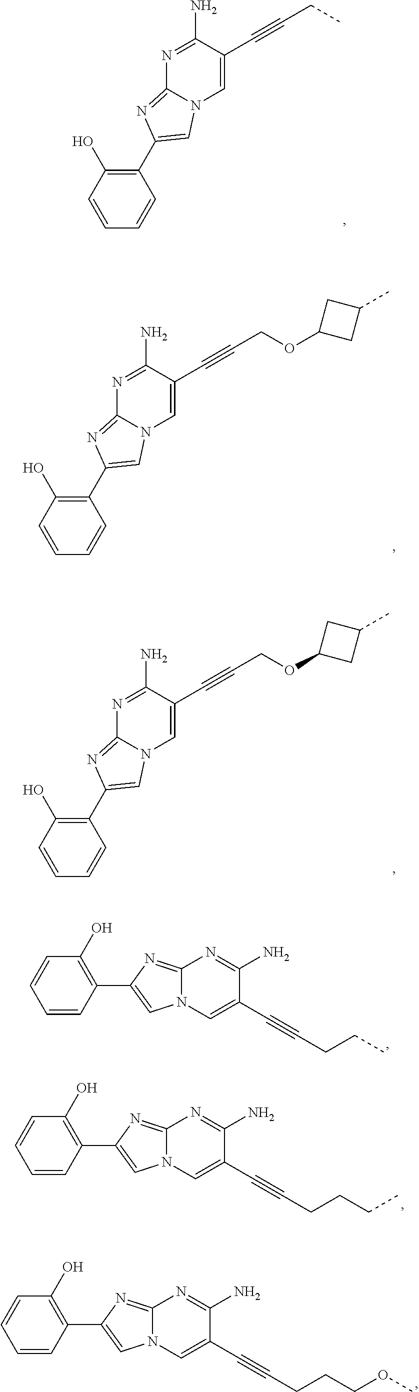

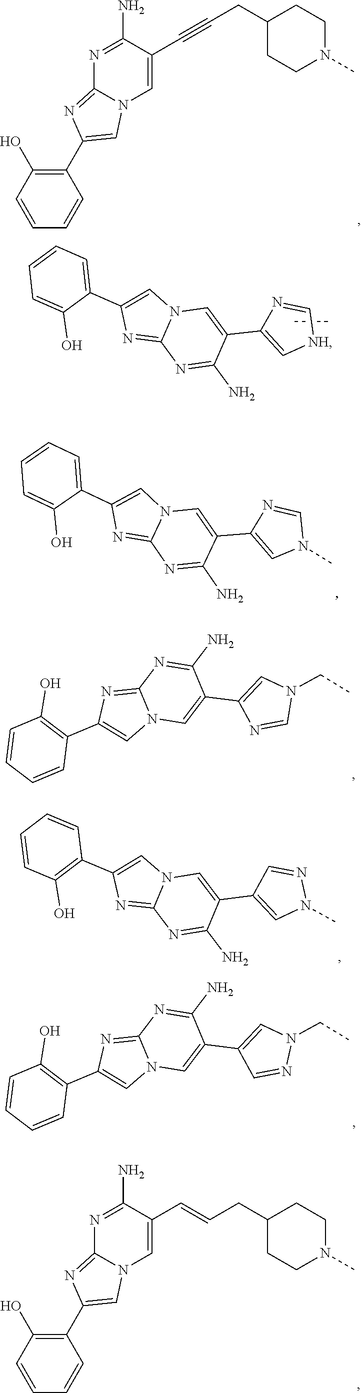

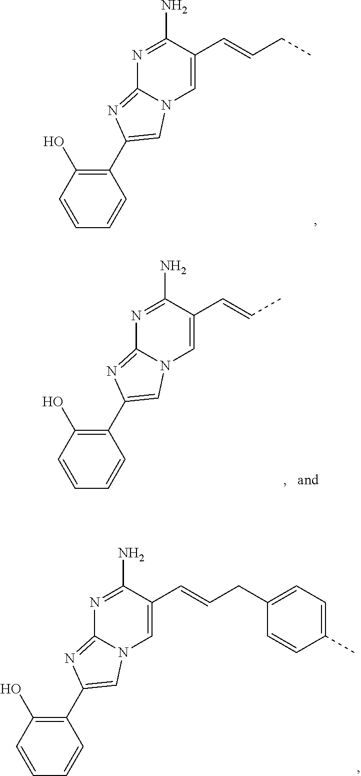

wherein [symbol] is the attachment point to the chemical linking moiety (L) (e.g., the chemical linking moiety is attached to a carbon of the indicated ring or a nitrogen of the indicated ring).

The compositions described herein exemplify some of the members of these types of small molecule target protein binding moieties. Such small molecule target protein binding moieties also include pharmaceutically acceptable salts, enantiomers, solvates and polymorphs of these compositions, as well as other small molecules that may target a protein of interest. References which are cited herein below are incorporated by reference herein in their entirety.

### Therapeutic Compositions

Disclosed herein are pharmaceutical compositions comprising at least one bifunctional compound as described herein in combination with a pharmaceutically effective amount of a carrier, additive or excipient. In any aspect or embodiment described herein, the pharmaceutical compositions disclosed herein further comprise an additional pharmaceutically active compound otherwise described herein.

The compositions of the present disclosure include, where applicable, the pharmaceutically acceptable salts, in particular, acid or base addition salts of compounds as described herein. Acids that can be used to prepare the pharmaceutically acceptable acid addition salts of the aforementioned base compounds and form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among numerous others.

Bases that may be used to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium, zinc, and magnesium), ammonium or water-soluble amine addition salts such as N-methylglucamine-(meglumine), and the lower alkanolammonium and other base salts of pharmaceutically acceptable organic amines, among others.

The compounds as described herein may, in accordance with the disclosure, be administered in single or divided doses by the oral, parenteral or topical routes. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Administration of compounds according to the present disclosure as sprays, mists, or aerosols for intra-nasal, intra-tracheal or pulmonary administration may also be used. The present disclosure therefore also is directed to pharmaceutical compositions comprising an effective amount of compound as described herein, optionally in combination with a pharmaceutically acceptable carrier, additive or excipient. Compounds according to the present disclosure may be administered in immediate release, intermediate release or sustained or controlled release forms. Sustained or controlled release forms are preferably administered orally, but also in suppository and transdermal or other topical forms. Intramuscular injections in liposomal form also may be used to control or sustain the release of compound at an injection site.

The compositions as described herein may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions as described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions as described herein may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions as described herein may be administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

The amount of compound in a pharmaceutical composition as described herein that may be combined with the carrier materials to produce a single dosage form will vary depending upon the host and disease treated and the particular mode of administration. Preferably, the compositions should be formulated to contain between about 0.05 milligram to about 750 milligrams or more, more preferably about 1 milligram to about 600 milligrams, and even more preferably about 10 milligrams to about 500 milligrams of active ingredient, alone or in combination with at least one other compound according to the present disclosure.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject in need of therapy using compounds according to the methods described herein can be treated by administering to the patient (subject) an effective amount of the compound according to the present disclosure including pharmaceutically acceptable salts, solvates or polymorphs, thereof optionally in a pharmaceutically acceptable carrier or diluent, either alone, or in combination with other known therapeutic agents as otherwise identified herein.

These compounds can be administered by any appropriate route, for example, orally, parenterally, intravenously, intradermally, subcutaneously.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably 0.1 to 100 mg/kg per day, more generally 0.5 to about 25 mg per kilogram body weight of the recipient/patient per day.

The compound is conveniently administered in any suitable unit dosage form, including but not limited to one containing less than 1 mg, 1 mg to 3000 mg, preferably 5 to 500 mg of active ingredient per unit dosage form. An oral dosage of about 25-250 mg is often convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as anti-cancer agents, including pembrolizumab, among others. In certain preferred aspects of the disclosure, one or more compounds according to the present disclosure are coadministered with another bioactive agent, such as an anti-cancer agent or a would healing agent, including an antibiotic, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

### Therapeutic Methods

In an additional aspect, the description provides therapeutic compositions comprising an effective amount of a compound as described herein or salt form thereof, and a pharmaceutically acceptable carrier. The therapeutic compositions modulate protein degradation in a patient or subject, for example, an animal such as a human, and can be used for treating or ameliorating disease states or conditions that are modulated through the degraded protein.

The terms “treat,” “treating,” and “treatment,” etc., as used herein, refer to any action providing a benefit to a patient for which the present compounds may be administered, including the treatment of any disease state or condition which is modulated through the protein to which the present compounds bind. Disease states or conditions, including cancer such as lung cancer, including non-small cell lung cancer, which may be treated using compounds according to the present disclosure are set forth hereinabove.

The description provides therapeutic compositions as described herein for effectuating the degradation of proteins of interest for the treatment or amelioration of a disease, e.g., cancer. In certain additional embodiments, the disease is multiple myeloma. As such, in another aspect, the description provides a method of ubiquitinating/degrading a target protein in a cell. In certain embodiments, the method comprises administering a bifunctional compound as described herein comprising, e.g., a ULM and a PTM, preferably linked through a linker moiety, as otherwise described herein, wherein the ULM is coupled to the PTM and wherein the ULM recognizes a ubiquitin pathway protein (e.g., an ubiquitin ligase, such as a VHL E3 ubiquitin ligase) and the PTM recognizes the target protein such that degradation of the target protein will occur when the target protein is placed in proximity to the ubiquitin ligase, thus resulting in degradation/inhibition of the effects of the target protein and the control of protein levels. The control of protein levels afforded by the present disclosure provides treatment of a disease state or condition, which is modulated through the target protein by lowering the level of that protein in the cell, e.g., cell of a patient. In certain embodiments, the method comprises administering an effective amount of a compound as described herein, optionally including a pharmaceutically acceptable excipient, carrier, adjuvant, another bioactive agent or combination thereof.

In additional embodiments, the description provides methods for treating or ameliorating a disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating the disease or disorder or symptom thereof in the subject.

In another aspect, the description provides methods for identifying the effects of the degradation of proteins of interest in a biological system using compounds according to the present disclosure.

In another embodiment, the present disclosure is directed to a method of treating a human patient in need for a disease state or condition modulated through a protein where the degradation of that protein will produce a therapeutic effect in the patient, the method comprising administering to a patient in need an effective amount of a compound according to the present disclosure, optionally in combination with another bioactive agent. The disease state or condition may be a disease caused by a microbial agent or other exogenous agent such as a virus, bacteria, fungus, protozoa, or other microbe or may be a disease state, which is caused by overexpression of a protein, which leads to a disease state and/or condition

The term “disease state or condition” is used to describe any disease state or condition wherein protein dysregulation (i.e., the amount of protein expressed in a patient is elevated) occurs and where degradation of one or more proteins in a patient may provide beneficial therapy or relief of symptoms to a patient in need thereof. In certain instances, the disease state or condition may be cured.

Exemplary disease states or conditions that may be treated using the disclosed bifunctional compounds include, for example, asthma, autoimmune diseases, such as multiple sclerosis, cancer, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease, (PKD1) or 4 (PKD2) Prader-Willi syndrome, Sickle-cell disease, Tay-Sachs disease, and Turner syndrome. In any aspect or embodiment described herein, the compounds disclosed herein are used to treat cancer.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present disclosure include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

The term “bioactive agent” is used to describe an agent, other than a compound according to the present disclosure, which is used in combination with the present compounds as an agent with biological activity to assist in effecting an intended therapy, inhibition and/or prevention/prophylaxis for which the present compounds are used. Preferred bioactive agents for use herein include those agents which have pharmacological activity similar to that for which the present compounds are used or administered and include for example, anti-cancer agents, antiviral agents, especially including anti-HIV agents and anti-HCV agents, antimicrobial agents, antifungal agents, etc.

The term “additional anti-cancer agent” is used to describe an anti-cancer agent, which may be combined with compounds according to the present disclosure to treat cancer. These agents include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitor, an AKT inhibitor, an mTORC1/2 inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIG 140, CC 8490, cilengitide, gimatecan, IL13-PE38QQR, INO 1001, IPdR₁ KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “pharmaceutically acceptable salt” is used throughout the specification to describe, where applicable, a salt form of one or more of the compounds described herein which are presented to increase the solubility of the compound in the gastic juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art. Sodium and potassium salts are particularly preferred as neutralization salts of the phosphates according to the present disclosure.

The term “pharmaceutically acceptable derivative” is used throughout the specification to describe any pharmaceutically acceptable prodrug form (such as an ester, amide other prodrug group), which, upon administration to a patient, provides directly or indirectly the present compound or an active metabolite of the present compound.

### EXEMPLIFICATION

### Abbreviations

ACN: acetonitrile
ADDP: 1,1′-(azodicarbonyl)dipiperidine
BAST: N,N-bis(2-methoxyethyl)aminosulfur trifluoride
BPO: benzoyl peroxide
Cbz: Carbonylbezyloxy
DAST: diethylaminosulfur trifluoride
DBE: 1,2-dibromoethane
DCM: dichloromethane
DEAD: diethyl azodicarboxylate
DIAD: diisopropyl azodicarboxylate
DIBAL: disiobutylaluminium hydride
DIEA or DIPEA: diisopropylethylamine
DMA: N,N-dimethylacetamide
DMF: N,N-dimethylformamide
DMP: Dess-Martin periodinane
EA: ethyl acetate
EDCI: 1-ethyl-3-(3-dimethylaminopropyl)carbodiimide
HBTU: N,N,N′N′-tetramethyl-O-(1H-benzotriazol-1-yl)uronium hexafluorophosphate
HMDS: bis9trimethylsilyl)amine
HMPA: hexamethylphosphoramide
LDA: lithium diisopropylamide
MCPBA: meta-chloroperoxybenzoic acid
MsCl: methanesulfonyl chloride
M.W: microwave
NBS: N-bromosuccinimide
NMP: N-methylpyrrolidone
PCC: pyridinium chlorochromate
Pd-118 or Pd(dtpf)Cl2: 1,1′-bis(di-tert-butylphosphino)ferrocene dichloropalladium
Pd(dppf)Cl2: 1,1′-bis(diphenylphosphino)ferrocene dichloropalladium
Pd(dba)2: bis(dibenzylideneacetone)palladium
Pd2(dba)3: Tris(dibenzylideneacetone)dipalladium
PPTS: pyridium p-tolunesulfonate
PTSA: p-toluenesulfonic acid
RuPhos-Pd-G3: XPhos-Pd-G3: [(2-dicyclohexylphosphino-2′,6′-diisopropoxy-1,1′-biphenyl)-2-(2′-amino-1,1′-biphenyl)] palladium(II) methanesulfonate
RuPhos-Pd-G2: Chloro[(2-dicyclohexylphosphino-2′,6′-diisopropoxy-1,1′-biphenyl)-2-(2′-amino-1,1′-biphenyl)] palladium(II)
SFC: supercritical fluid chromatography t-BuXPhos-Pd-G3: [(2-di-tert-butylphosphino-2′,4′,6′-triisopropyl-1,1′-biphenyl)-2-(2′-amino-1,1′-biphenyl)] palladium(II) methanesulfonate
TEA: trimethylamine
TFA: trifluoroacetic acid
TLC: thin layer chromatography
TMP: 2,2,6,6-tetramethylpiperidine
TEMPO: 2,2,6,6-tetramethylpiperidine-N-oxide
TosC1 or TsCl: p-toluenesulfonyl chloride
TsOH: p-toluenesulfonic acid
XantPhos: 4,5-bis(diphenylphosphino)-9,9-dimethylxanthene
XPhos: 2-dicyclohexylphosphino-2′4′6′-triisopropylbiphenyl
XPhos-Pd-G3: [(2-dicyclohexylphosphino-2′,4′,6′-triisopropyl-1,1′-biphenyl)-2-(2′-amino-1,1′-biphenyl)] palladium(II) methanesulfonate
12354-85-7: bis(pentamethylcyclopentadienylrhodium dichloride)

### General Synthetic Approaches

The PTM represented by Formulas I, IV, V, VIII, and IX can be synthesized by following the general synthetic routes detailed in the schemes below.

In the case of PTMs represented by Formula I exemplary general synthetic approaches are outlined in the scheme below (equally applicable to PTMs where W_{PTM3A} is used in place of W_{PTM3} and/or W_{PTM5A} is used in place of W_{PTM5}):

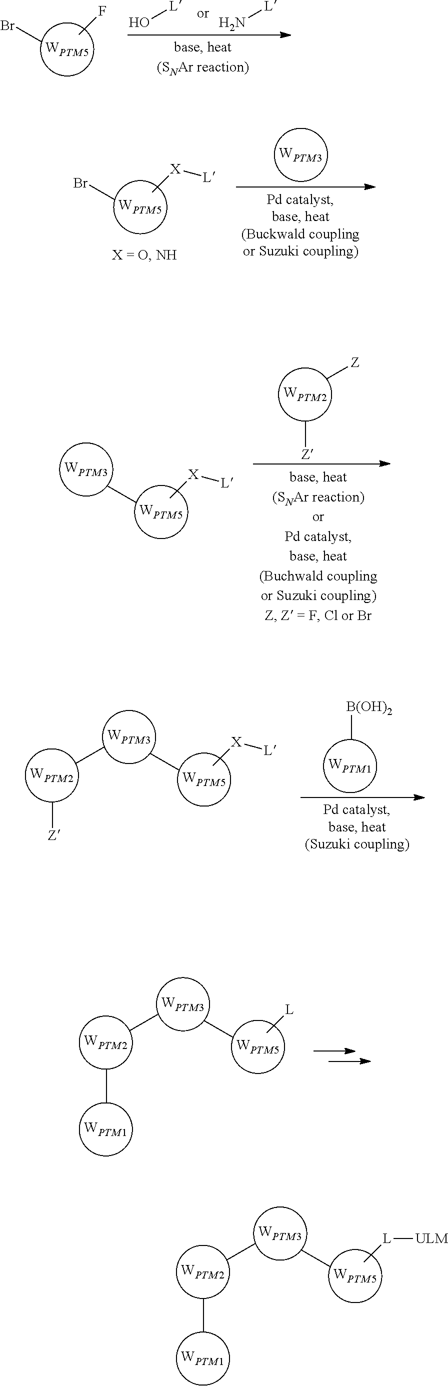

One skilled in the art would recognize that Buchwald coupling or S_{N}Ar approach in the scheme above is applicable when W_{PTM3} (or W_{PTM3A}) is a heterocycloalkyl connected to W_{PTM2} and W_{PTM5} through N atoms in W_{PTM3} (or W_{PTM3A}). Otherwise, alternative transition metal-catalyzed coupling approaches (e.g., Suzuki coupling) would be more relevant (for example, if W_{PTM3} (or W_{PTM3A}) is an aryl or a heteroaryl).

One skilled in the art would also recognize that Buchwald coupling, or S_{N}Ar, or Suzuki coupling approach described in the scheme above would be mostly applicable when W_{PTM5} (or W_{PTM5A}) is an aryl or a heteroaryl. Otherwise, in cases where W_{PTM5} (or W_{PTM5A}) is a heterocycloalkyl the exact approach would depend on the nature of the functional group present in the above said heterocycloalkyl. Examples of possible approaches (reductive amination, nucleophilic substitution) are provided in the scheme below.

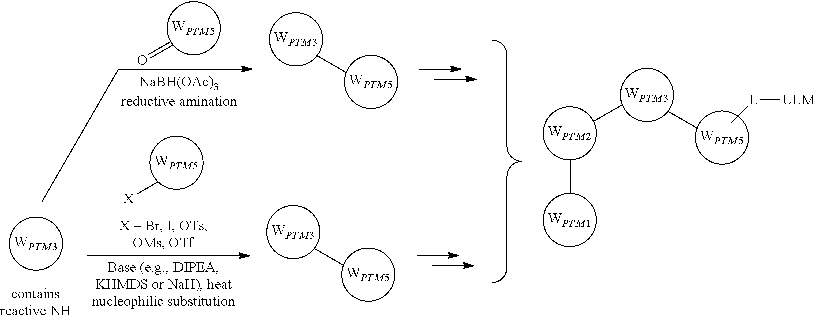

In the case where PTMs are represented by the general Formula V, and W_{PTM1} and W_{PTM2} are more specifically defined, the compounds can be synthesized as described in the scheme below. One skilled in the art would appreciate that alternative catalysts, temperature, solvent, and other experimental conditions compatible with S_{N}Ar reactions and transition metal-mediated coupling reactions can also be used.

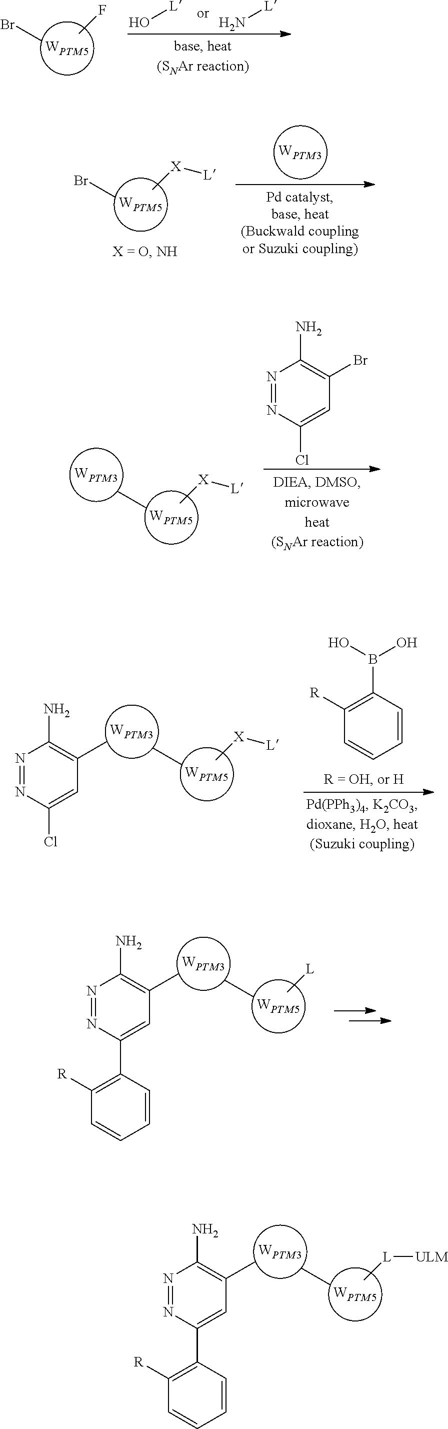

One skilled in the art would also appreciate that a modified approach can be utilized to enable attachment of PTMs via different chemical linkers. For example, in cases where W_{PTM5} is connected to L′ via a CH₂ group (X═CH₂ in the schemes above) one can envision an approach described in the scheme below:

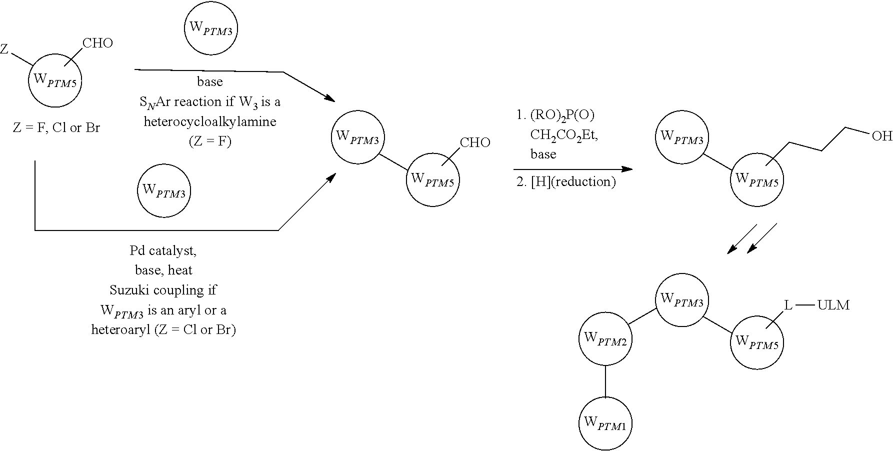

Alternatively, if W_{PTM5} is not present, PTM of an exemplary bifunctional compounds represented by Formula I can be synthesized according to the general scheme below if a reactive NH is present in W_{PTM3} (W_{PTM3A}). Alternatively, attaching linker to W_{PTM3} (W_{PTM3A}) can be achieved using approaches described above for W_{PTM5}.

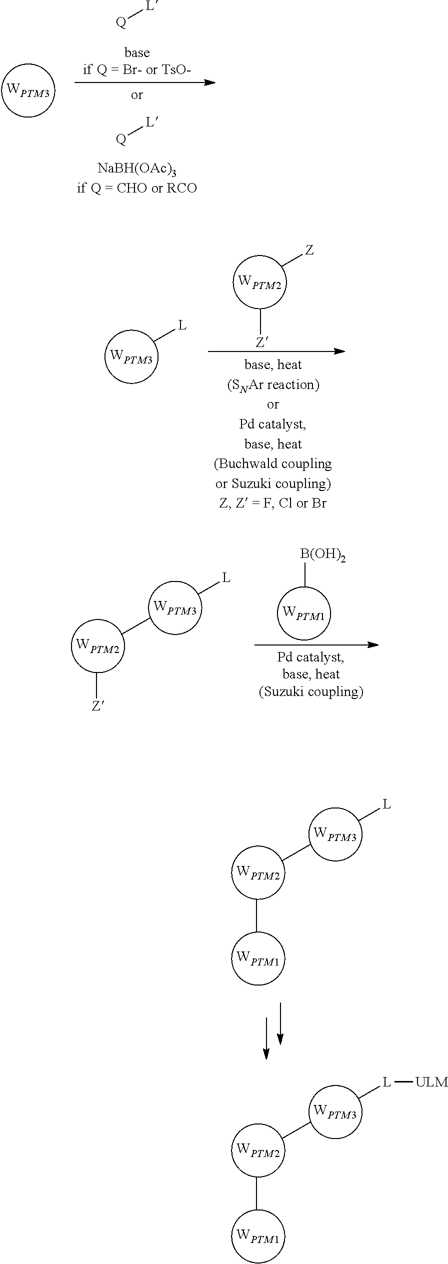

One skilled in the art will appreciate that synthetic approaches described herein can be modified to adapt to the specific nature of the W_{PTM1}, W_{PTM2}, W_{PTM3}, W_{PTM3A}, W_{PTM5}, and W_{PTM5A} rings.

PTMs represented by general Formula IVa can be prepared according to the following general scheme (the description would similarly apply to W_{PTM5A}):

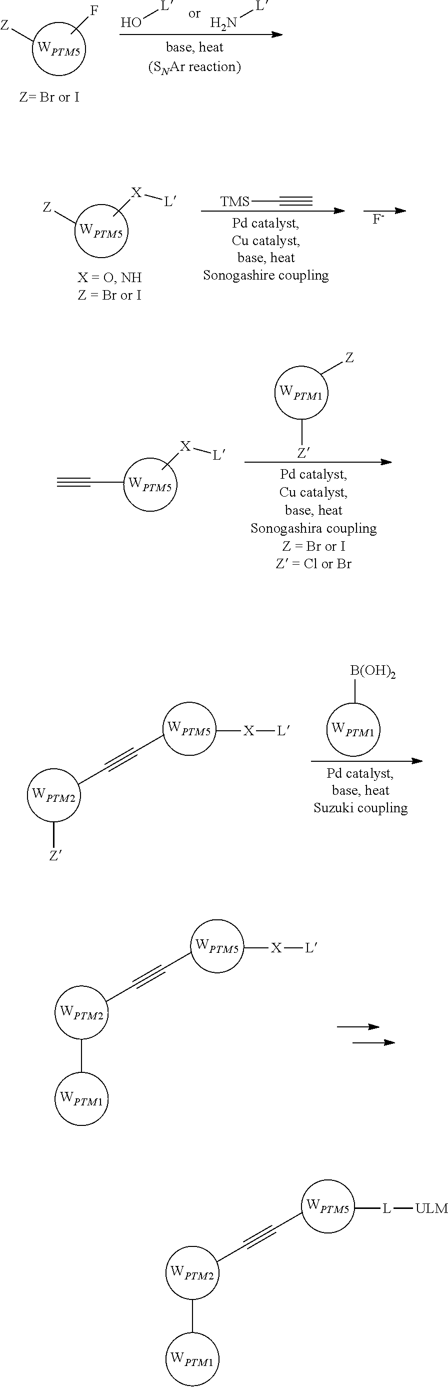

One skilled in the art will appreciate that the approach to the generation of W_{PTM5}-connected alkyne shown in the scheme above is most applicable when W_{PTM5} is an aryl or a heteroaryl. One skilled in the art will also appreciate that in cases where W_{PTM5} is a cycloalkyl, or a heterocycloalkyl, an alternative approach to the generation of the alkyne can be employed—for example, one based on the Ohira-Bestmann reagent shown in the scheme below.

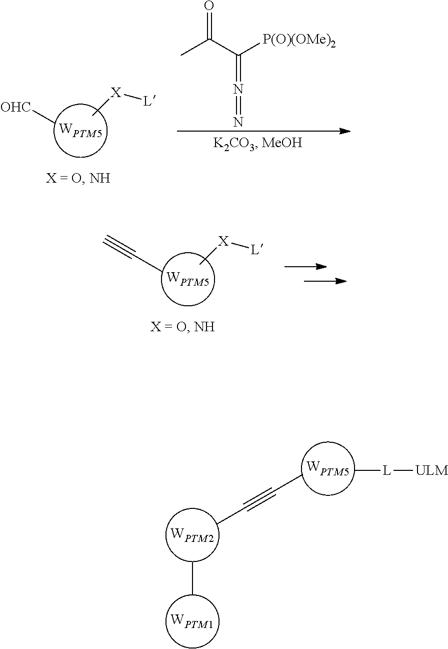

In addition, PTMs represented by general Formulas IVb1 and IVb2 can be prepared, in one possible approach, according to the following general scheme:

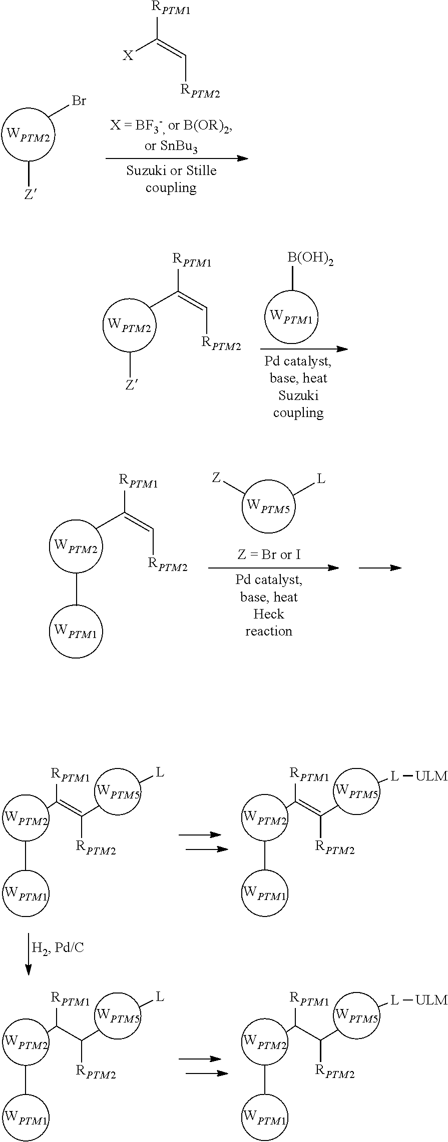

One skilled in the art will recognize that in many cases the applicable synthetic approaches would depend on the exact nature of the components comprising the PTM moiety, as well as the nature of connectivity between them. For example, in cases where W_{PTM3} contains a reactive NH functionality a number of synthetic approaches relevant to the preparation of PTMs described by general formula VII can be envisioned, some nonlimiting examples of which are described in the scheme below:

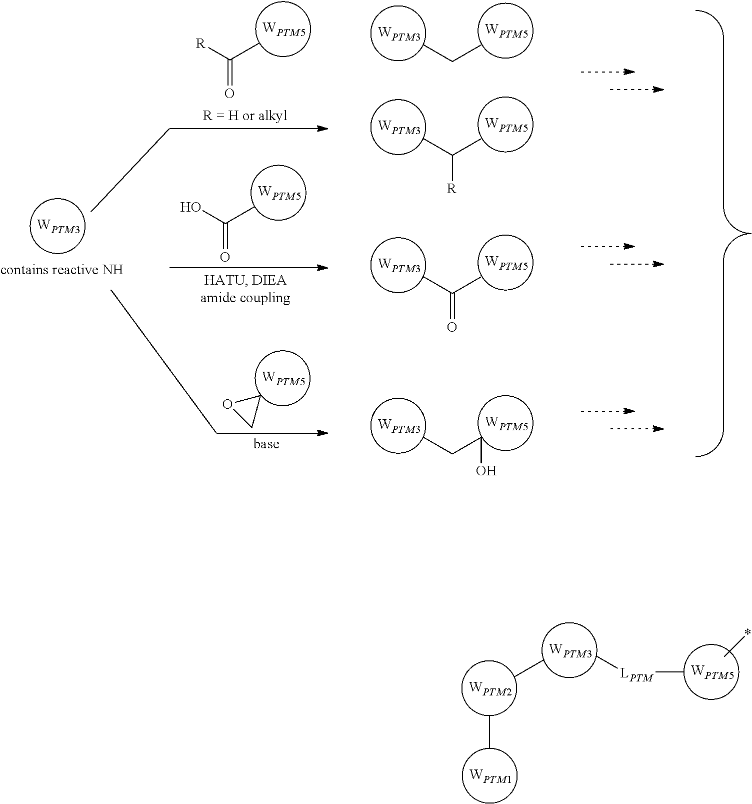

PTMs represented by general Formulas IXa and IXb can be prepared, in at least one example, through general approaches detailed in the schemes below:

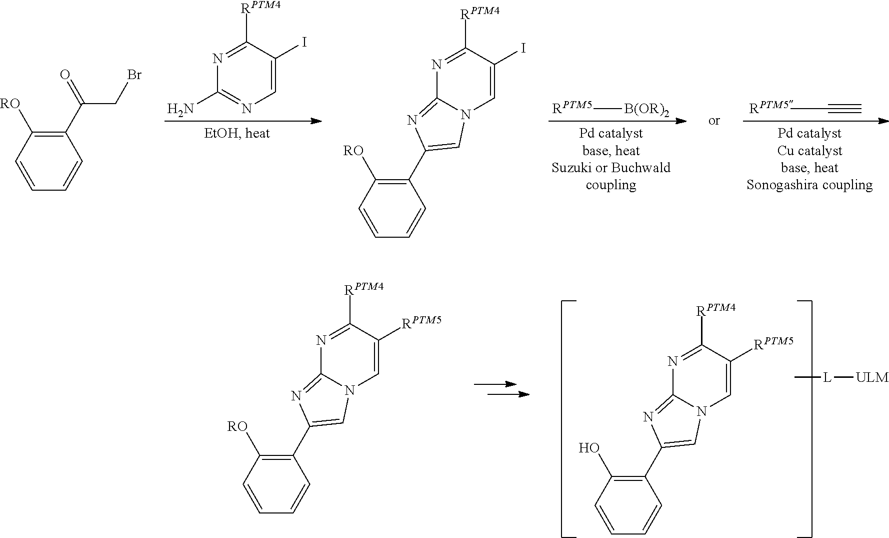

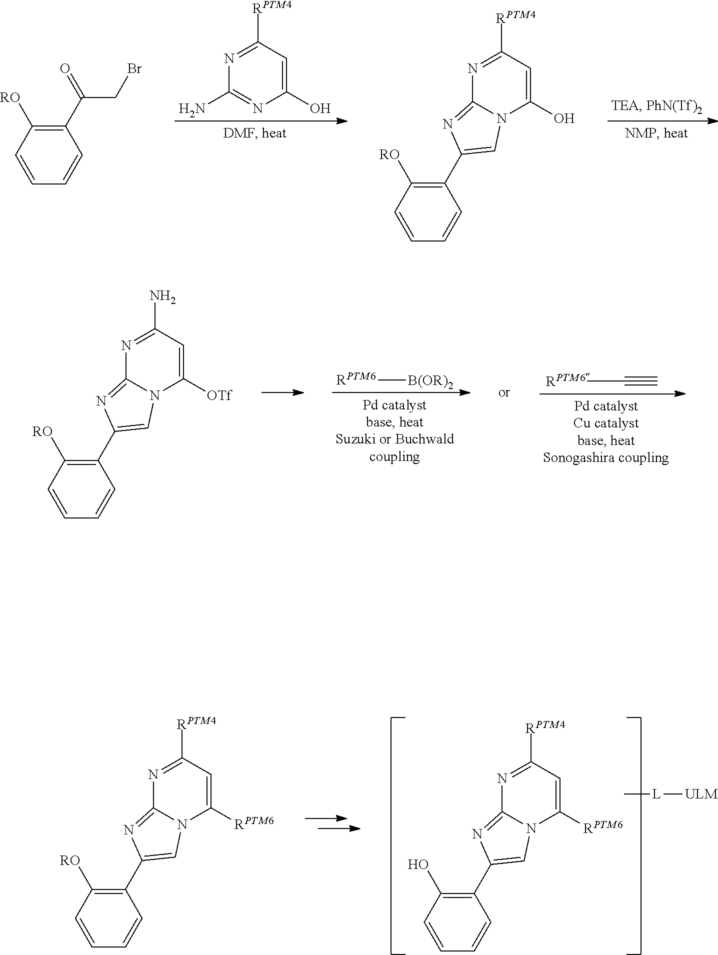

One skilled in the art will recognize that bifunctional compounds of the current disclosure can be prepared using various possible sequences of steps. In some embodiments, the bifunctional compounds of the current disclosure are assembled by joining together two fragments via a final connection in the middle of the linker using synthetic methods as shown in schemes below, such as, for example, in the preferred general method A.

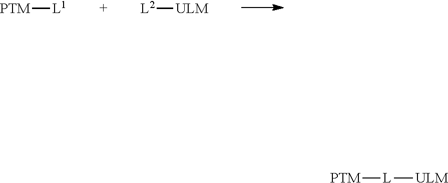

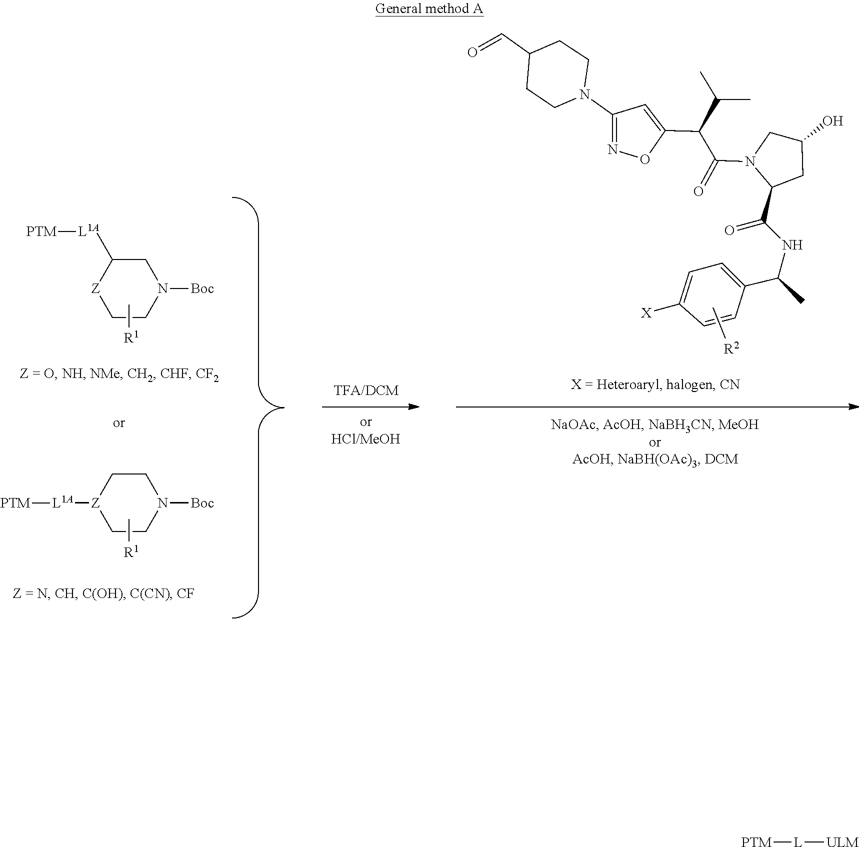

In some embodiments, the PTM-L^{1A}-heterocycloalkyl motif referenced in method A can be assembled by introducing a connection in L^{1A} as shown in general methods B and C.

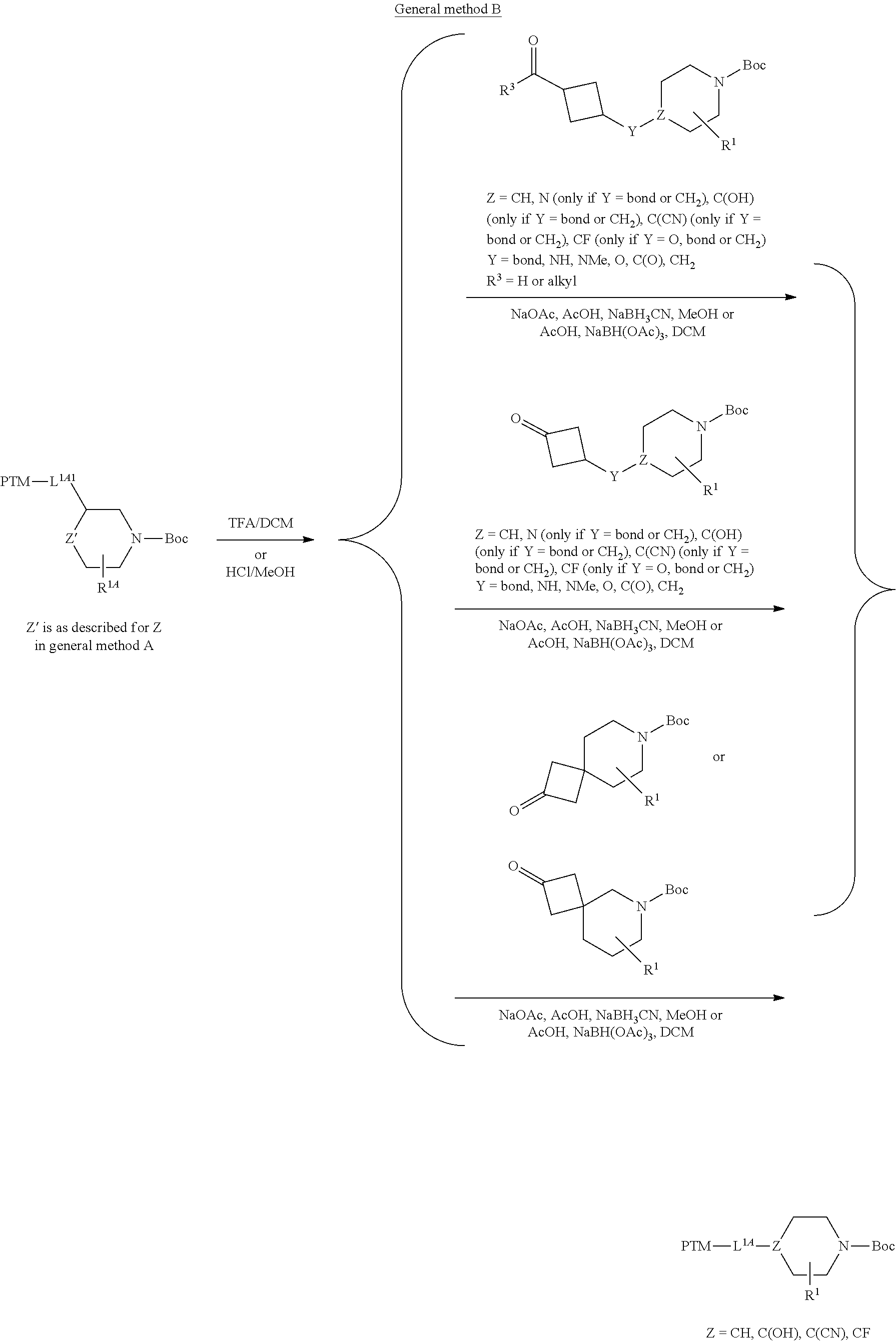

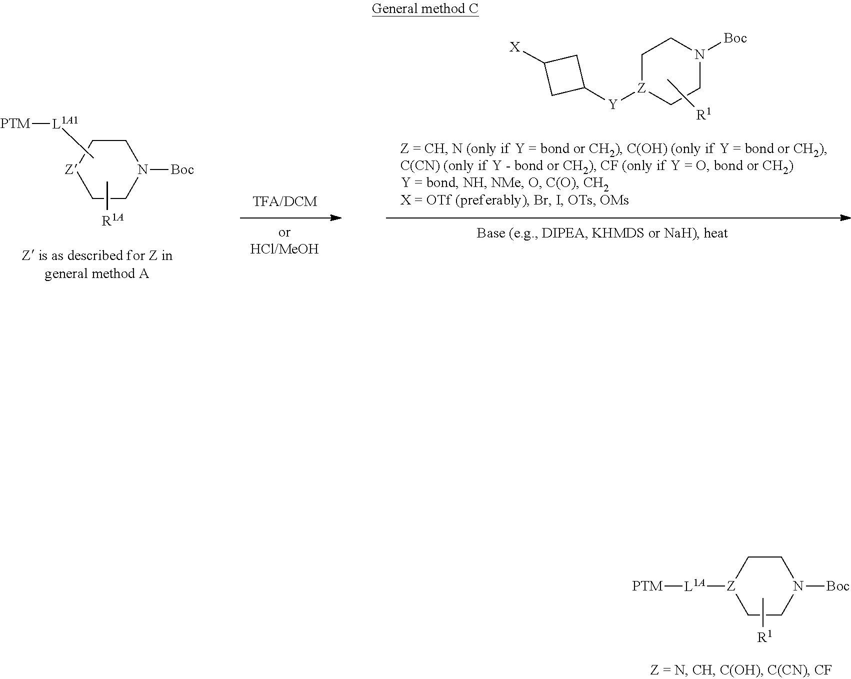

Those skilled in the art will appreciated that certain protecting groups can be used interchangeably in the context of general methods A, B and C. For example, Cbz protecting group may be used in place of Boc, in which case alternative methods of its cleavage (e.g., TMSI/ACN or Pd/C, H₂) may be employed.

Additionally, one skilled in the art will recognize that heterocycloalkyls in general methods A, B and C are also meant to include, by extension, heterocycloalkyls with ring sizes different from the ones explicitly shown, and that the nature of heterocycloalkyls is also meant to include, by extension, heteroaryls with reactive NH functionality. For example, in some embodiments, the above said heteroaryl can be an optionally substituted imidazole or an optionally substituted pyrazole. The above said heteroaryls can be subjected to the reductive amination or nucleophilic substitution conditions described in methods A, B, and C. One skilled in the art will also recognize that the above said heteroaryls may require different protecting groups (e.g., SEM) other than those shown in the scheme above.

In some embodiments, the final connection can be introduced between the two parts of the ULM motif as shown in the scheme below for general method D.

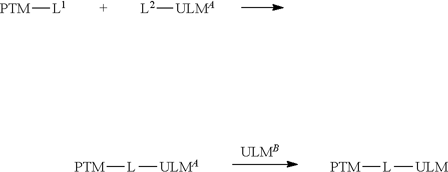

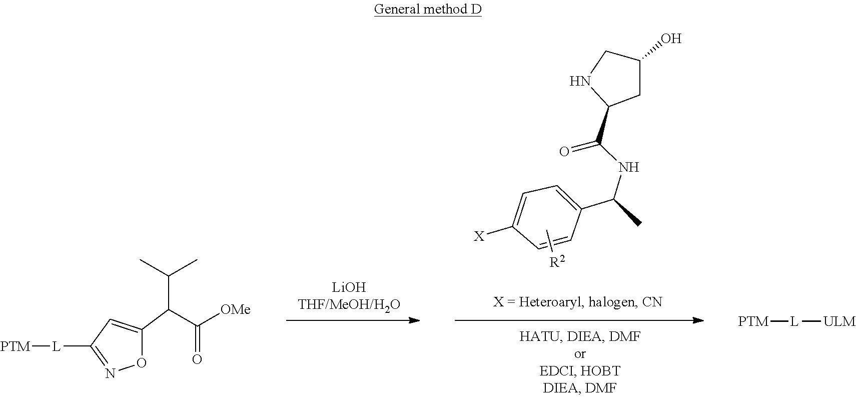

The synthetic realization and optimization of the bifunctional molecules as described herein may be approached in a step-wise or modular fashion. For example, identification of compounds that bind to the target molecules can involve high or medium throughput screening campaigns if no suitable ligands are immediately available. It is not unusual for initial ligands to require iterative design and optimization cycles to improve suboptimal aspects as identified by data from suitable in vitro and pharmacological and/or ADMET assays. Part of the optimization/SAR campaign would be to probe positions of the ligand that are tolerant of substitution and that might be suitable places on which to attach the linker chemistry previously referred to herein. Where crystallographic or NMR structural data are available, these can be used to focus such a synthetic effort.

In a very analogous way one can identify and optimize ligands for an E3 Ligase, i.e. ULMs/VLMs.

With PTMs and ULMs (e.g. VLMs) in hand, one skilled in the art can use known synthetic methods for their combination with or without a linker moiety. Linker moieties can be synthesized with a range of compositions, lengths and flexibility and functionalized such that the PTM and ULM groups can be attached sequentially to distal ends of the linker. Thus, a library of bifunctional molecules can be realized and profiled in in vitro and in vivo pharmacological and ADMET/PK studies. As with the PTM and ULM groups, the final bifunctional molecules can be subject to iterative design and optimization cycles in order to identify molecules with desirable properties.

In some instances, protecting group strategies and/or functional group interconversions (FGIs) may be required to facilitate the preparation of the desired materials. Such chemical processes are well known to the synthetic organic chemist and many of these may be found in texts such as “Greene's Protective Groups in Organic Synthesis” Peter G. M. Wuts and Theodora W. Greene (Wiley), and “Organic Synthesis: The Disconnection Approach” Stuart Warren and Paul Wyatt (Wiley).

### Exemplary Synthesis of Compound BRM1

### Step 1

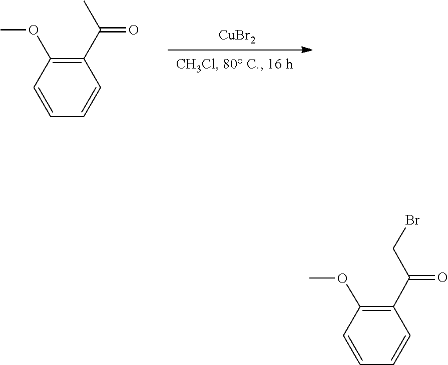

A mixture of CuBr₂ (2.53 g, 11.32 mmol) and 2′-methoxyacetophenone (1.0 g, 6.66 mmol) in chloroform (20 mL) was stirred for 16 hours at 80° C. The reaction was filtered and concentrated. The residue was purified by flash chromatography (0-10% ethyl acetate in petroleum ether) to afford the title compound (860 mg, 56%) as a colorless oil.

### Step 2

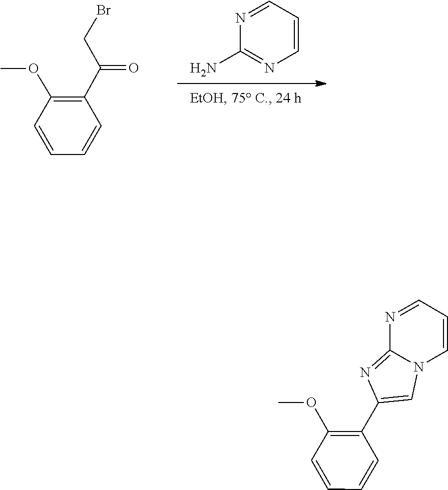 [SMILES: COc1ccccc1C(=O)CBr ; Nc1ncccn1 ; COc1ccccc1-c1cn2cccnc2n1]

The mixture of 2-pyrimidinamine (0.28 g, 2.97 mmol) and 2-bromo-1-(2-methoxyphenyl)ethanone (0.4 g, 1.75 mmol) in ethanol (10 mL) was stirred for 16 hours at 80° C. The mixture was filtered, and the residue was concentrated to afford the crude title compound (220 mg) as a yellow solid.

### Step 3

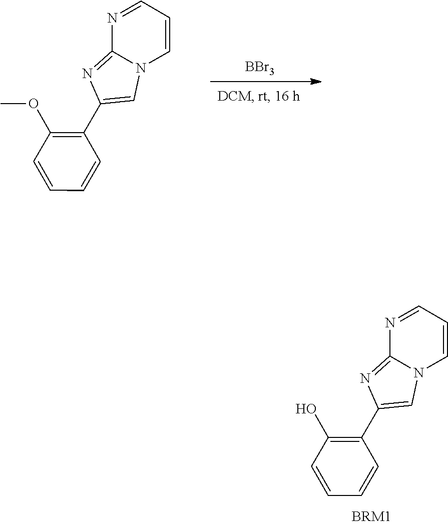 [SMILES: COc1ccccc1-c1cn2cccnc2n1 ; "BBr3" ; Oc1ccccc1-c1cn2cccnc2n1]

To a solution of 2-(2-methoxyphenyl)imidazo[1,2-a]pyrimidine (0.2 g, 0.83 mmol) in dichloromethane (5 mL) was added tribromoborane (0.35 g, 1.42 mmol) at 20° C. The mixture was stirred for 16 hours at 80° C. The mixture was purified by Xtimate C18 chromatography 150*25 mm*5 um (acetonitrile 18%/0.1% FA in water) to afford the title compound (7.3 mg) as a yellow solid.

### Exemplary Synthesis of Compound BRM2

### Step 1

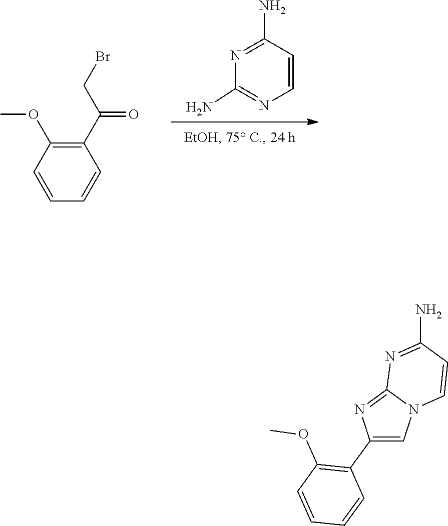 [SMILES: COc1ccccc1C(=O)CBr ; Nc1ccnc(N)n1 ; COc1ccccc1-c1cn2ccc(N)nc2n1]

The mixture of 2,4-pyrimidinediamine (0.33 g, 2.97 mmol) and 2-bromo-1-(2-methoxyphenyl)ethanone (0.4 g, 1.75 mmol) in ethanol (10 mL) was stirred for 16 hours at 80° C. The mixture was filtered, and the residue was concentrated to afford the crude title compound (220 mg) as a white solid.

### Step 2

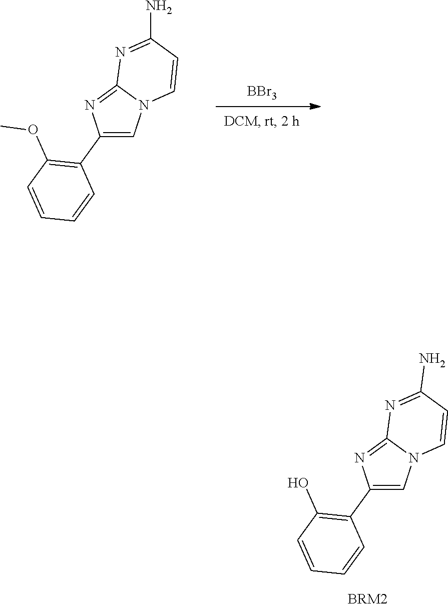

The mixture of tribromoborane (0.18 g, 0.71 mmol) and 2-(2-methoxyphenyl)imidazo[1,2-a]pyrimidin-7-amine (0.1 g, 0.42 mmol) in dichloromethane (2 mL) was stirred for 16 hours at 20° C. The solution was purified by Xtimate C18 chromatography 150*25 mm*5 um (acetonitrile 18%/0.225% FA in water) to afford the title compound (68.6 mg) as a white solid.

### Exemplary Synthesis of Compound BRM3

### Step 1

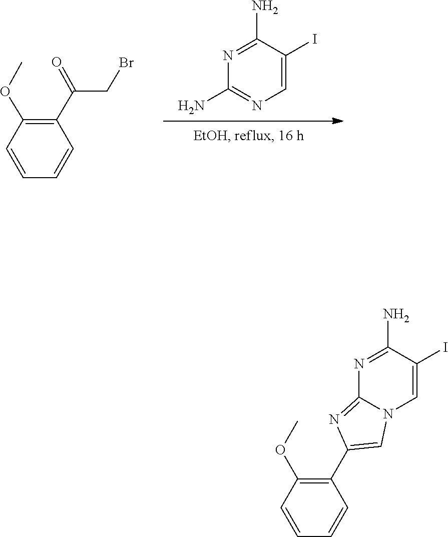 [SMILES: COc1ccccc1C(=O)CBr ; Nc1ncc(I)c(N)n1 ; COc1ccccc1-c1cn2cc(I)c(N)nc2n1]

A solution of 5-iodopyrimidine-2,4-diamine (103 mg, 0.44 mmol) and 2-bromo-1-(2-methoxyphenyl)ethanone (100.0 mg, 0.44 mmol) in ethanol (1 mL) was stirred at 75° C. for 16 hours. Formation of white precipitate was observed, and the reaction was filtered to afford the title compound (120 mg) as a white solid.

### Step 2

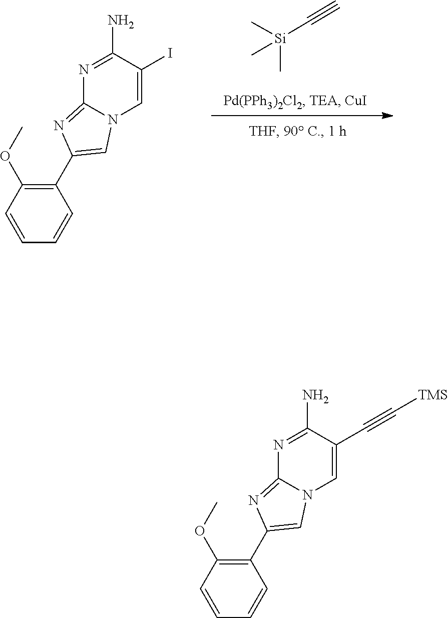

To a solution of Pd(PPh₃)₂Cl₂ (19.2 mg, 0.03 mmol) in tetrahydrofuran (3 mL) was added 6-iodo-2-(2-methoxyphenyl)imidazo[1,2-a]pyrimidin-7-amine (100.0 mg, 0.27 mmol), CuI (5.2 mg, 0.03 mmol), TEA (0.15 mL, 1.09 mmol) and trimethylsilylacetylene (0.05 mL, 0.33 mmol) at 20° C. The mixture was stirred at 60° C. for 3 hours under N₂ atmosphere. The mixture was filtered and concentrated. The residue was purified by column chromatography on silica gel (0-5% in petroleum ether) to give the title compound (65 mg) as a light yellow solid.

### Step 3

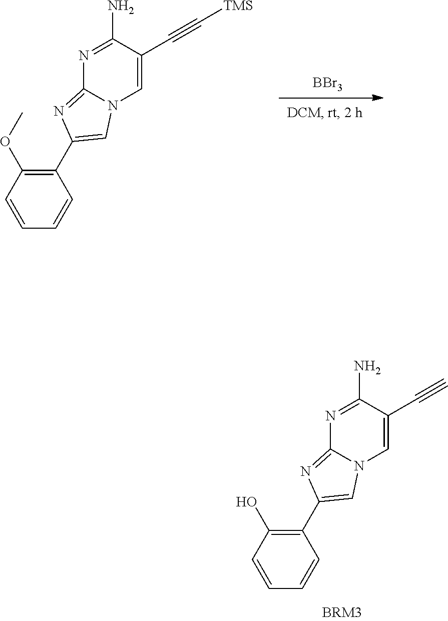 [SMILES: COc1ccccc1-c1cn2cc(C#C[Si](C)(C)C)c(N)nc2n1 ; "BBr3" ; C#Cc1cn2cc(-c3ccccc3O)nc2nc1N]

To a stirred solution of 2-[7-amino-6-(2-trimethylsilylethynyl)imidazo[1,2-a]pyrimidin-2-yl]phenol (50.0 mg, 0.1600 mmol) in tetrahydrofuran (8 mL) was added 1M BBr₃ (0.31 mL, 0.31 mmol) in DCM. The mixture was stirred at 25° C. for 15 hours under nitrogen atmosphere. The reaction mixture was concentrated, and then the residue was diluted with MeOH and purified by pre-HPLC: Xtimate C18 150*25 mm*5 um (acetonitrile 10-15%/0.225% FA in water) to give the title compound as a white solid.

### Exemplary Synthesis of Compound BRM5

### Step 1

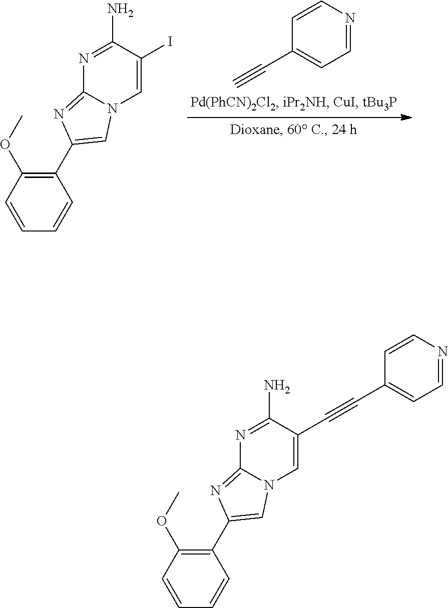

To a solution of 6-iodo-2-(2-methoxyphenyl)imidazo[1,2-a]pyrimidin-7-amine (100.0 mg, 0.27 mmol) in 1,4-dioxane (1 mL) was added CuI (5.2 mg, 0.03 mmol), tri-tert-butylphosphine (16.58 mg, 0.08 mmol), Pd(PhCN)₂Cl₂ (10.5 mg, 0.03 mmol), diisopropylamine (82.9 mg, 0.82 mmol) and 4-ethynylpyridine (33.8 mg, 0.33 mmol). The mixture was stirred at 60° C. for 24 hours under N2 atmosphere. The mixture was filtered and concentrated. The residue was purified by column chromatography on silica gel (0-5% MeOH in dichloromethane) to give the title compound (60 mg) as a light yellow solid.

### Step 2

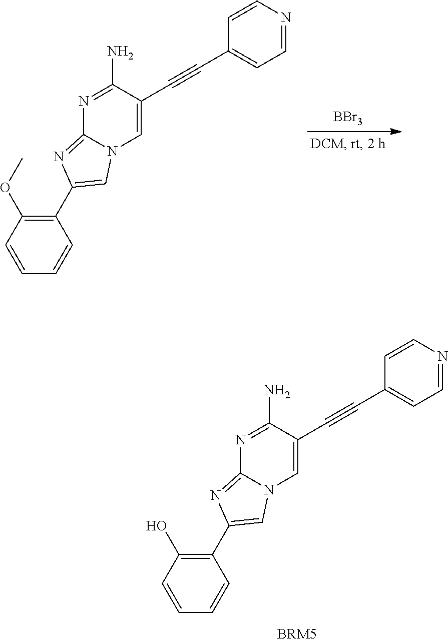

To a solution of 2-(2-methoxyphenyl)-6-[2-(4-pyridyl)ethynyl]imidazo[1,2-a]pyrimidin-7-amine (0.05 g, 0.15 mmol) in DCM (20 mL) was added tribromoborane (0.04 mL, 0.16 mmol). The reaction was stirred at 20° C. for 16 hours. The reaction mixture was concentrated, and the resulting residue was purified by silica gel column chromatography (20-50% EtOAc in petroleum ether) to afford the title compound (14.7 mg) as a yellow solid.

### Exemplary Synthesis of Compound BRM4

### Step 1

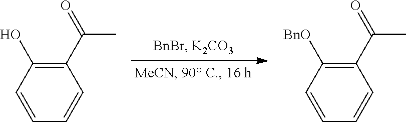

To a mixture of 1-(2-hydroxyphenyl)ethanone (10 g, 73.45 mmol, 8.9 mL, 1 eq) and benzylbromide (12.56 g, 73.45 mmol, 8.7 mL, 1 eq) in acetonitrile (100 mL) was added potassium carbonate (20.30 g, 146.90 mmol, 2 eq) and potassium iodide (1.22 g, 7.34 mmol, 0.1 eq) in one portion at 20° C. under nitrogen. The mixture was stirred at 90° C. for 16 hours. The reaction mixture was quenched by water (80 mL) and concentrated to remove acetonitrile, then extracted with ethyl acetate (50 mL×3). The combined organic phase was washed with saturated brine (20 mL), dried with anhydrous sodium sulfate, filtered and concentrated in vacuum. The residue was purified by silica gel chromatography (petroleum ether/ethyl acetate=50/1 to 10/1) to afford 1-(2-benzyloxyphenyl)ethanone (17.1 g) as a colorless oil.

### Step 2

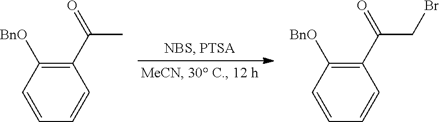

To a solution of 1-(2-benzyloxyphenyl)ethanone (16.1 g, 71.15 mmol, 1 eq) in acetonitrile (240 mL) was added N-bromosuccinimide (16.46 g, 92.50 mmol, 1.3 eq) and p-toluenesulfonic acid (1.23 g, 7.12 mmol, 0.1 eq), and the mixture was stirred at 30° C. for 16 hours. The mixture was poured into water (200 mL), and the aqueous phase was extracted with ethyl acetate (200 mL×2). The combined organic phase was washed with brine (150 mL×2), dried with anhydrous sodium sulfate, filtered and concentrated in vacuum. The residue was purified by silica gel chromatography (petroleum ether/ethyl acetate=100/1 to 30/1) to afford 1-(2-benzyloxyphenyl)-2-bromo-ethanone (20.5 g, 67.18 mmol, 94% yield) as a yellow solid.

### Step 3

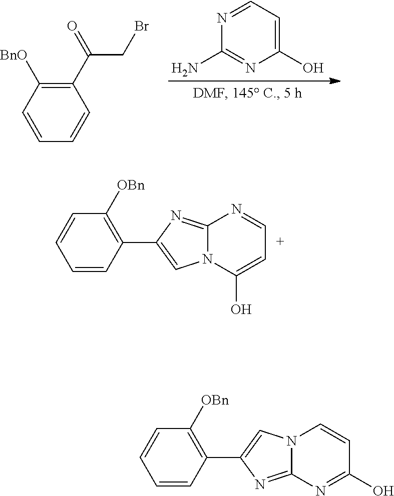 [SMILES: O=C(CBr)c1ccccc1OCc1ccccc1 ; Nc1nccc(O)n1 ; Oc1ccnc2nc(-c3ccccc3OCc3ccccc3)cn12 ; Oc1ccn2cc(-c3ccccc3OCc3ccccc3)nc2n1]

A solution of 1-(2-benzyloxyphenyl)-2-bromo-ethanone (15 g, 49.15 mmol, 1 eq) and 2-aminopyrimidin-4-ol (5.46 g, 49.15 mmol, 1 eq) in N,N-dimethylformamide (150 mL) was stirred at 145° C. for 4 hours. The mixture was cooled to 20° C. and poured into ice water (1000 mL), The precipitate was filtered and dried in vacuo. The material was purified by semi-preparative reverse phase HPLC (column: Phenomenex Synergi Max-RP 250*80 mm*10 um; mobile phase: [water (0.1% TFA)-ACN]; B %: 25%-55%, 20 min), followed by an additional semi-preparative reverse phase HPLC (column: Phenomenex Luna C18 200*40 mm*10 um; mobile phase: [water (0.1% TFA)-ACN];B %: 32%-52%, 10 min). Compounds 2-(2-benzyloxyphenyl)imidazo[1,2-a]pyrimidin-5-ol (130 mg, 0.40 mmol) and 2-(2-benzyloxyphenyl)imidazo[1,2-a]pyrimidin-7-ol (6.5 g, 20.48 mmol) were obtained as white solids.

### Step 4

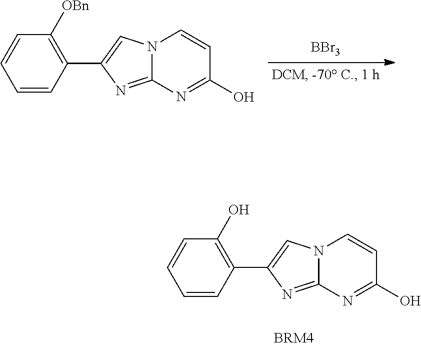 [SMILES: Oc1ccn2cc(-c3ccccc3OCc3ccccc3)nc2n1 ; "BBr3" ; Oc1ccn2cc(-c3ccccc3O)nc2n1]

To a solution of 2-(2-benzyloxyphenyl)imidazo[1,2-a]pyrimidin-7-ol (200 mg, 0.63 mmol, 1 eq) in dichloromethane (20 mL) was added boron tribromide (157 mg, 0.63 mmol, 1 eq) at −70° C. under nitrogen. The mixture was stirred at −70° C. for 30 minutes. The reaction mixture was quenched with methanol and concentrated under reduced pressure at 45° C. The residue was purified by semi-preparative reverse phase HPLC (column: Luna C18 150*25 5 u; mobile phase: [water (0.1% TFA)-ACN]; B %: 5%-35%, 12 min) to give 2-(2-hydroxyphenyl) imidazo[1,2-a]pyrimidin-7-ol trifluoroacetate (100.3 mg, 0.293 mmol) as a white solid.

### Exemplary Synthesis of Compound BRM6

### Step 1

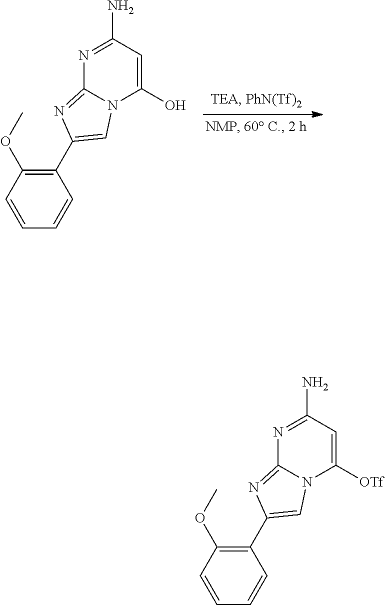 [SMILES: COc1ccccc1-c1cn2c(O)cc(N)nc2n1 ; COc1ccccc1-c1cn2c(OS(=O)(=O)C(F)(F)F)cc(N)nc2n1]

To a solution of 7-amino-2-(2-methoxyphenyl)imidazo[1,2-a]pyrimidin-5-ol (500 mg, 1.95 mmol) in 1-methyl-2-pyrrolidinone (8 mL) was added TEA (0.3 mL, 2.15 mmol) and N-phenylbis(trifluoromethanesulfonimide) (697 mg, 1.95 mmol). The mixture was stirred at 60° C. for 2 hours. The mixture was diluted with water (10 mL) and extracted with EtOAc (30 mL×3). The combined organic layers were washed with water (10 mL×3) and brine (15 mL), dried over anhydrous Na₂SO₄, filtered and concentrated. The residue was purified by column chromatography on silica gel (0-100% THF in DCM) to give the title compound (270 mg) as a light yellow solid.

### Step 2

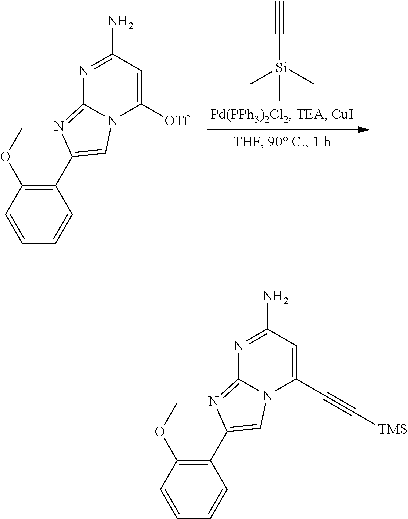

To a solution of [7-amino-2-(2-methoxyphenyl)imidazo[1,2-a]pyrimidin-5-yl]trifluoromethanesulfonate (30.0 mg, 0.08 mmol) in tetrahydrofuran (1 mL) was added trimethylsilylacetylene (0.02 mL, 0.12 mmol), Pd(PPh₃)₂Cl₂ (10.8 mg, 0.02 mmol), CuI (2.94 mg, 0.02 mmol) and triethylamine (0.04 mL, 0.31 mmol) at 20° C. The mixture was stirred at 60° C. for 3 hours under N2 atmosphere. The mixture was concentrated. The residue was purified by column chromatography on silica gel (0-5% MeOH in DCM) to give the title compound (18 mg) as a light yellow solid.

### Step 3

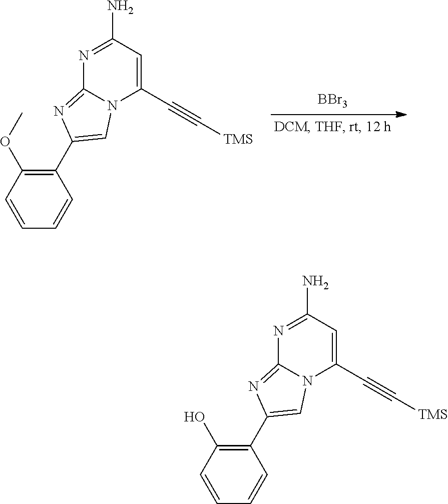 [SMILES: COc1ccccc1-c1cn2c(C#C[Si](C)(C)C)cc(N)nc2n1 ; "BBr3" ; C[Si](C)(C)C#Cc1cc(N)nc2nc(-c3ccccc3O)cn12]

To a solution of 2-(2-methoxyphenyl)-5-(2-trimethylsilylethynyl)imidazo[1,2-a]pyrimidin-7-amine (18 mg, 0.05 mmol) in dichloromethane (1 mL) was added tribromoborane (0.25 mL, 2.67 mmol). The mixture was stirred at 25° C. for 16 hours. The reaction was quenched with MeOH (2 mL) and concentrated to give the title compound (17 mg) as a yellow oil.

### Step 4

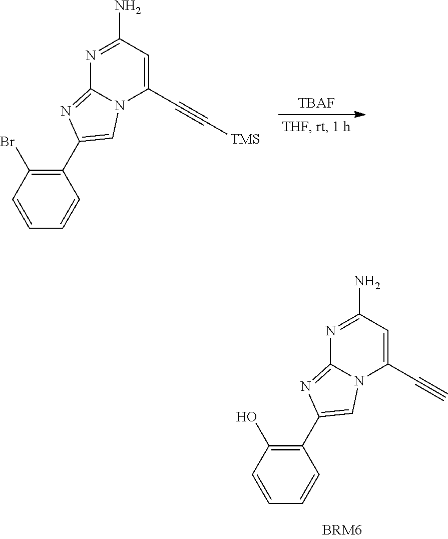 [SMILES: C[Si](C)(C)C#Cc1cc(N)nc2nc(-c3ccccc3Br)cn12 ; "TBAF" ; C#Cc1cc(N)nc2nc(-c3ccccc3O)cn12]

To a solution of 2-[7-amino-5-(2-trimethylsilylethynyl)imidazo[1,2-a]pyrimidin-2-yl]phenol (20.0 mg, 0.06 mmol) in tetrahydrofuran (0.5 mL) was added tetrabutylammoniumfluoride (0.19 mL, 0.19 mmol) in THF. The mixture was stirred at 25° C. for 16 hours. The reaction was concentrated, and the residue was purified by Xtimate C18 150*25 mm*5 um (acetonitrile 30-60%/0.2% formic acid in water) to give the title compound (0.5 mg) as a yellow solid.

### Exemplary Synthesis of Compound BRM8

### Step 1

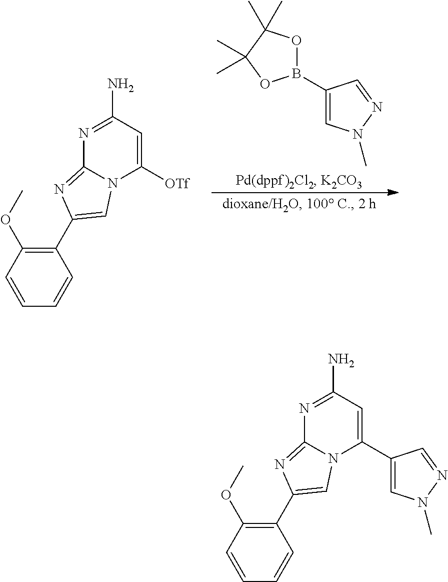 [SMILES: COc1ccccc1-c1cn2c(OS(=O)(=O)C(F)(F)F)cc(N)nc2n1 ; Cn1cc(B2OC(C)(C)C(C)(C)O2)cn1 ; "Pd(dppf)2Cl2, K2CO3" ; COc1ccccc1-c1cn2c(-c3cnn(C)c3)cc(N)nc2n1]

To a solution of 7-amino-2-(2-methoxyphenyl)imidazo[1,2-a]pyrimidin-5-yl trifluoromethanesulfonate (30.0 mg, 0.08 mmol) in 1,4-dioxane (1 mL) and water (0.2 mL) was added 1-methyl-4-(4,4,5,5-tetramethyl-1,3,2-dioxaborolan-2-yl)-1H-pyrazole (24 mg, 0.12 mmol), Pd(PPh₃)₂Cl₂ (10.8 mg, 0.02 mmol), and K₂CO₃ (32 mg, 0.23 mmol) at 20° C. The mixture was stirred at 100° C. for 3 hours under N2 atmosphere. The mixture was concentrated. The residue was purified by column chromatography on silica gel (0-10% in petroleum ether) to give the title compound (18 mg) as a light yellow solid.

### Step 2

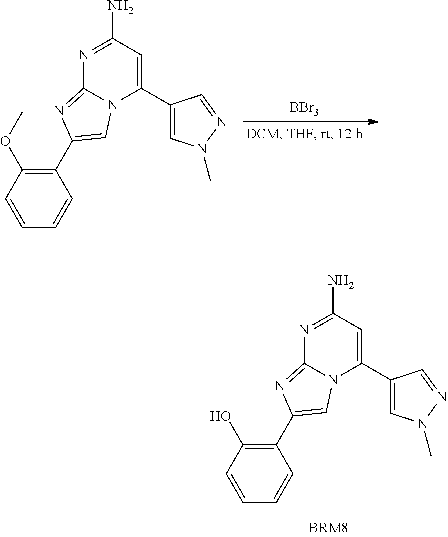 [SMILES: COc1ccccc1-c1cn2c(-c3cnn(C)c3)cc(N)nc2n1 ; "BBr3" ; Cn1cc(-c2cc(N)nc3nc(-c4ccccc4O)cn23)cn1]

To a solution of 2-(2-methoxyphenyl)-5-(1-methyl-1H-pyrazol-4-yl)imidazo[1,2-a]pyrimidin-7-amine (18 mg, 0.06 mmol) in DCM (1 mL) was added 1M tribromoborane (0.27 mL, 2.81 mmol) in DCM. The mixture was stirred at 25° C. for 16 hours. The reaction was quenched with MeOH (3 mL) and concentrated to give the title compound (7 mg) as a white solid.

### Exemplary Synthesis of Compound BRM9

### Step 1

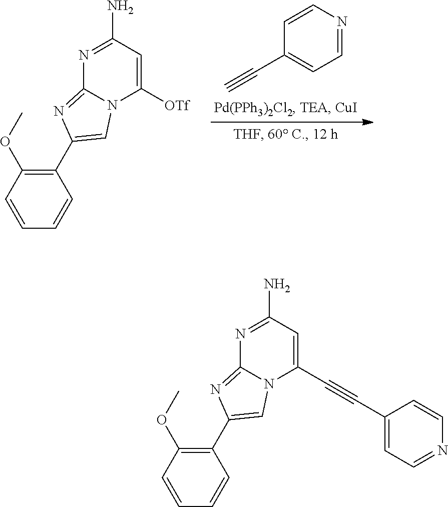

To a solution of 7-amino-2-(2-methoxyphenyl)imidazo[1,2-a]pyrimidin-5-yl trifluoromethanesulfonate (50.0 mg, 0.13 mmol) in tetrahydrofuran (1.5 mL) was added 4-ethynylpyridine (0.03 mL, 0.19 mmol), Pd(PPh₃)₂Cl₂ (18.08 mg, 0.03 mmol), CuI (4.9 mg, 0.03 mmol) and TEA (0.07 mL, 0.52 mmol) at 20° C. The mixture was stirred at 60° C. for 16 hours under N2 atmosphere. The mixture was filtered and concentrated. The residue was purified by column chromatography on silica gel (0-5% MeOH in DCM) to give the title compound (35 mg) as a light yellow solid.

### Step 2

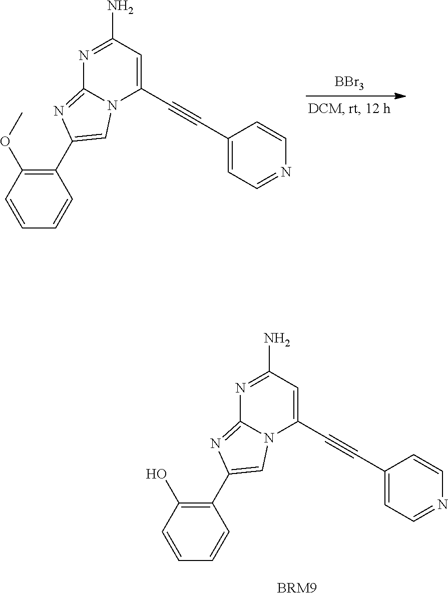 [SMILES: COc1ccccc1-c1cn2c(C#Cc3ccncc3)cc(N)nc2n1 ; "BBr3" ; Nc1cc(C#Cc2ccncc2)n2cc(-c3ccccc3O)nc2n1]

To a solution of 2-(2-methoxyphenyl)-5-[2-(4-pyridyl)ethynyl]imidazo[1,2-a]pyrimidin-7-amine (50.0 mg, 0.15 mmol) in dichloromethane (3 mL) was added tribromoborane (0.69 mL, 7.32 mmol). The mixture was stirred at 25° C. for 16 hours. Quenched with MeOH (3 mL), the mixture was concentrated. The residue was purified by Xtimate C18 150*25 mm*5 um (acetonitrile 30-60%/10 mM NH₄HCO₃ in water) to give the title compound (2.9 mg) as a light yellow solid.

### Exemplary Synthesis of Compound BRM10

### Step 1

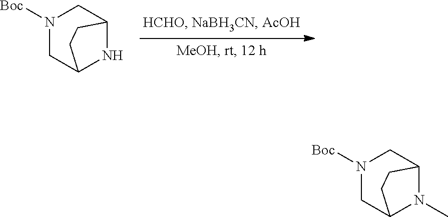 [SMILES: CC(C)(C)OC(=O)N1CC2CCC(C1)N2 ; CN1C2CCC1CN(C(=O)OC(C)(C)C)C2]

To a solution of tert-butyl 3,8-diazabicyclo[3.2.1]octane-3-carboxylate (1.0 g, 4.71 mmol) in methyl alcohol (20 mL) was added formaldehyde (1.4 g, 47.11 mmol) followed by acetic acid (0.05 mL, 0.94 mmol) and sodium cyanoborohydride (888 mg, 14.13 mmol). The mixture was stirred at 40° C. for 12 hours. The mixture was treated with TEA (0.5 mL) and concentrated to dryness. The residue was purified by column chromatography on silica gel (0-3% MeOH in DCM) to give the title compound (600 mg) as a colorless oil.

### Step 2

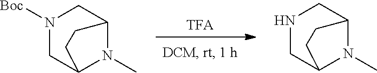

To a solution of tert-butyl 8-methyl-3,8-diazabicyclo[3.2.1]octane-3-carboxylate (200.0 mg, 0.88 mmol) in dichloromethane (3 mL) was added 2,2,2-trifluoroacetic acid (1.97 mL, 26.51 mmol). The mixture was stirred at 15° C. for 2 hours. The mixture was concentrated to give the crude title compound (310 mg, trifluoroacetate) as a light yellow oil.

### Step 3

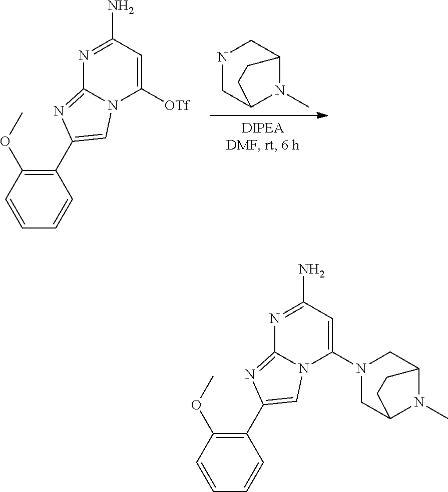

To a solution of [7-amino-2-(2-methoxyphenyl)imidazo[1,2-a]pyrimidin-5-yl]trifluoromethanesulfonate (100.0 mg, 0.26 mmol) in N,N-dimethylformamide (3 mL) was added 8-methyl-3,8-diazabicyclo[3.2.1]octane trifluoroacetate (310.0 mg, 0.88 mmol) and N,N-diisopropylethylamine (0.43 mL, 2.58 mmol). The mixture was stirred at 80° C. for 12 hours. The mixture was concentrated, and the residue was purified by column chromatography on silica gel (0-10% MeOH in DCM, 1% NH₄OH) to give the title compound (70 mg) as a light yellow solid.

### Step 4

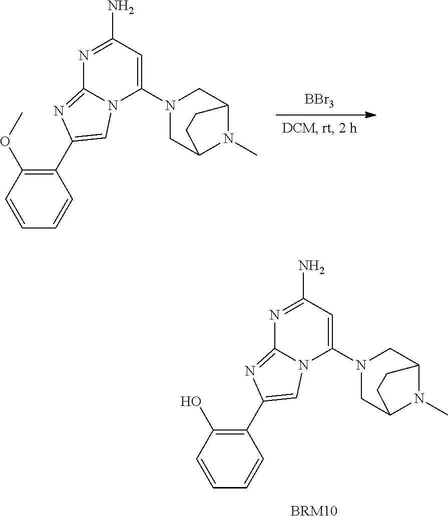 [SMILES: COc1ccccc1-c1cn2c(N3CC4CCC(C3)N4C)cc(N)nc2n1 ; "BBr3" ; "DCM, rt, 2 h" ; CN1C2CCC1CN(c1cc(N)nc3nc(-c4ccccc4O)cn13)C2]

To a solution of 2-(2-methoxyphenyl)-5-(8-methyl-3,8-diazabicyclo[3.2.1]octan-3-yl)imidazo[1,2-a]pyrimidin-7-amine (70.0 mg, 0.19 mmol) in DCM (5 mL) was added tribromoborane (1M, 0.91 mL, 9.6 mmol) in DCM. The mixture was stirred at 25° C. for 16 hours. The reaction mixture was quenched with MeOH (3 mL) at −78° C. and concentrated. The residue was purified by Xtimate C18 150*25 mm*5 um (acetonitrile 0-25%/0.2% formic acid in water) to give the title compound (35.6 mg) as a white solid.

Using analogous procedures the Exemplary Compound BRM7 was synthesized.

### Exemplary Synthesis of Compound BRM11

### Step 1

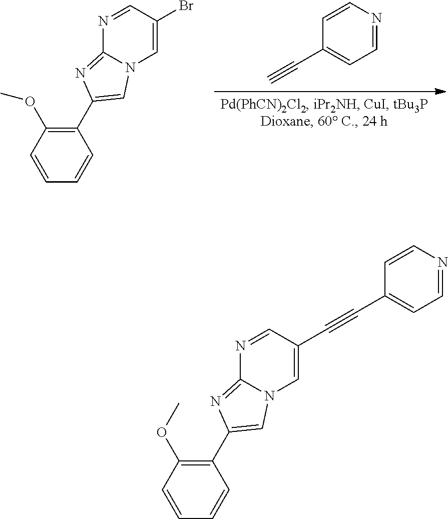

To a solution of 6-bromo-2-(2-methoxyphenyl)imidazo[1,2-a]pyrimidine (1 g, 3.29 mmol) in 1,4-dioxane (50 mL) was added CuI (62.6 mg, 0.33 mmol), tBu₃P (199.5 mg, 0.99 mmol), Pd(PhCN)₂Cl₂ (126.1 mg, 0.33 mmol), iPr₂NH (998.1 mg, 9.86 mmol) and 4-ethynylpyridine (406.9 mg, 3.95 mmol). The mixture was stirred at 60° C. for 24 hours under N2 atmosphere. The mixture was filtered and concentrated. The residue was purified by column chromatography on silica gel (0-5% MeOH in dichloromethane) to give the title compound (600 mg) as a light yellow solid.

### Step 2

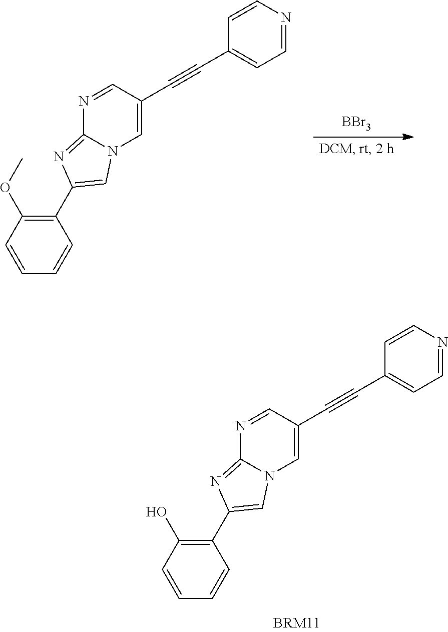 [SMILES: COc1ccccc1-c1cn2cc(C#Cc3ccncc3)cnc2n1 ; "BBr3" ; "DCM, rt, 2 h" ; Oc1ccccc1-c1cn2cc(C#Cc3ccncc3)cnc2n1]

To a solution of 2-(2-methoxyphenyl)-6-[2-(4-pyridyl)ethynyl]imidazo[1,2-a]pyrimidine (600.0 mg, 1.84 mmol) in DCM (20 mL) was added tribromoborane (1.74 mL, 18.39 mmol), and the resulted mixture was stirred at 80° C. for 16 hours. The reaction was quenched by MeOH (20 mL) and poured into ice-water (50 mL). The mixture was adjusted to pH=7 with saturated NaHCO₃ solution and extracted with ethyl acetate (50 mL×2). The combined organic layers were washed with brine (30 mL), dried with Na₂SO₄. The mixture was concentrated, and the residue was purified by pre-HPLC: Xtimate C18 150*25 mm*5 um (acetonitrile 12-15%/0.225% FA in water) to give the title compound (230.5 mg) as a yellow solid.

### Exemplary Synthesis of Compound BRM12

### Step 1

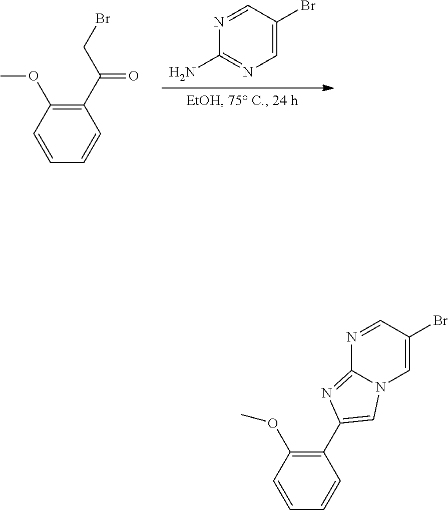

A solution of 5-bromo-2-pyrimidinamine (3.27 g, 18.77 mmol) and 2-bromo-1-(2-methoxyphenyl)ethanone (4.3 g, 18.77 mmol) in ethanol (25 mL) was stirred at 75° C. for 16 hours. White precipitate formation was observed. The reaction was filtered to collect the title compound (4 g) as a white solid.

### Step 2

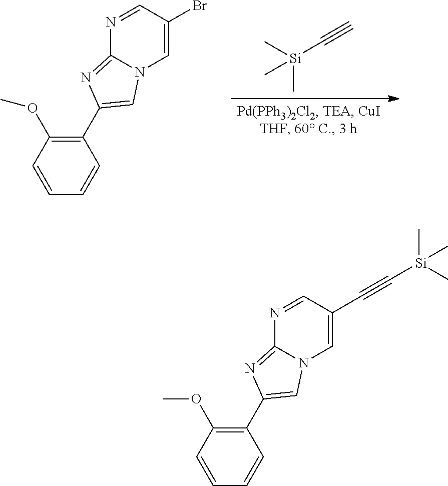

To a solution of Pd(PPh3)₂Cl₂ (230 mg, 0.33 mmol) in tetrahydrofuran (50 mL) was added 6-bromo-2-(2-methoxyphenyl)imidazo[1,2-a]pyrimidine (1.0 g, 3.29 mmol), CuI (62 mg, 0.33 mmol), TEA (1.8 mL, 13 mmol) and ethynyltrimethylsilane (0.55 mL, 3.95 mmol) at 20° C. The mixture was stirred at 60° C. for 3 hours under N2 atmosphere. The mixture was filtered and concentrated. The residue was purified by column chromatography on silica gel (0-5% EtOAc in petroleum ether) to give the title compound (280 mg) as a light yellow solid.

### Step 3

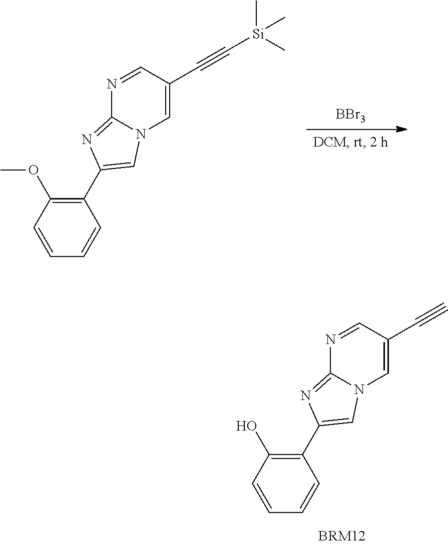

To a solution of 2-[2-(2-methoxyphenyl)imidazo[1,2-a]pyrimidin-6-yl]ethynyl-trimethyl-silane (200.0 mg, 0.62 mmol) in dichloromethane (4.2 mL) was added tribromoborane (0.59 mL, 6.22 mmol), the resulted mixture was stirred at 80° C. for 16 hours. The reaction was quenched by MeOH (20 mL) and poured into ice-water (50 mL), and the mixture was adjusted to pH=7 with saturated NaHCO₃ solution and extracted with ethyl acetate (50 mL×2). The combined organic layers were washed with brine (30 mL), dried with Na₂SO₄. The mixture was concentrated, and the residue was purified by pre-HPLC Xtimate C18 150*25 mm*5 um (acetonitrile 12-15%/0.225% FA in water) to afford the title compound (18.7 mg) as a yellow solid.

### Exemplary Synthesis of Compound BRM13

### Step 1

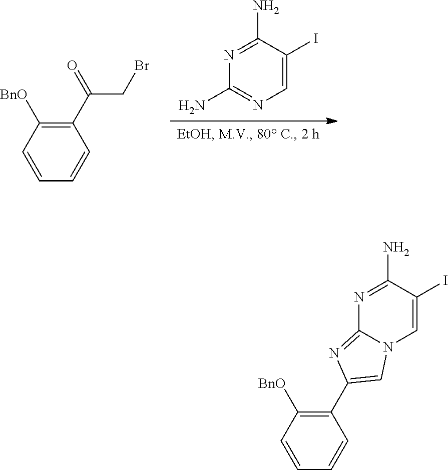

1-(2-benzyloxyphenyl)-2-bromoethanone (142 mg, 0.47 mmol, 1.1 eq) and 5-iodopyrimidine-2,4-diamine (100 mg, 0.42 mmol, 1 eq) were taken up in a microwave tube in ethanol (2 mL). The sealed tube was heated at 80° C. for 2 hours under microwave irradiation. The mixture was concentrated under reduced pressure at 45° C. The residue was purified by semi-preparative reverse phase HPLC (column: Phenomenex Gemini-NX C18 75*30 mm*3 um; mobile phase: [water (0.1% TFA)-ACN]; B %: 18%-48%, 7 min). Compound 2-(2-benzyloxyphenyl)-6-iodo-imidazo[1,2-a]pyrimidin-7-amine (73 mg, 0.16 mmol) was obtained as a white solid.

### Step 2

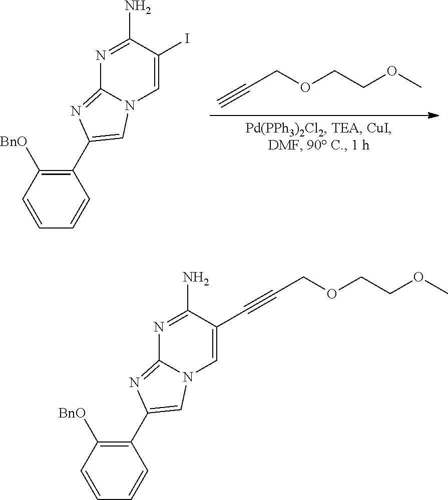

To a mixture of 2-(2-benzyloxyphenyl)-6-iodo-imidazo[1,2-a]pyrimidin-7-amine (70 mg, 0.15 mmol, 1 eq), CuI (6 mg, 0.03 mmol, 0.2 eq), triethylamine (48 mg, 0.47 mmol, 3 eq) and 3-(2-methoxyethoxy)prop-1-yne (27 mg, 0.24 mmol, 1.5 eq) in N,N-dimethylformamide (2 mL) was added [1,1′-bis(diphenylphosphino)ferrocene]dichloropalladium (ii) (11 mg, 0.02 mmol, 0.1 eq) in one portion at 0° C. under nitrogen. The mixture was stirred at 90° C. for 2 hours. The mixture was cooled to 20° C. and concentrated under reduced pressure at 45° C. The residue was purified by semi-preparative reverse phase HPLC (column: Phenomenex Gemini-NX C18 75*30 mm*3 um; mobile phase: [water (0.1% TFA)-ACN]; B %: 30%-40%, 7 min). Compound 2-(2-benzyloxyphenyl)-6-[3-(2-methoxyethoxy)prop-1-ynyl]imidazo[1,2-a]pyrimidin-7-amine (35 mg, 0.08 mmol) was obtained as a white solid.

2-(2-benzyloxyphenyl)-6-[3-(2-methoxyethoxy)prop-1-ynyl]imidazo[1,2-a]pyrimidin-7-amine was converted to the title compound according to the scheme below using procedure described for example BRM4.

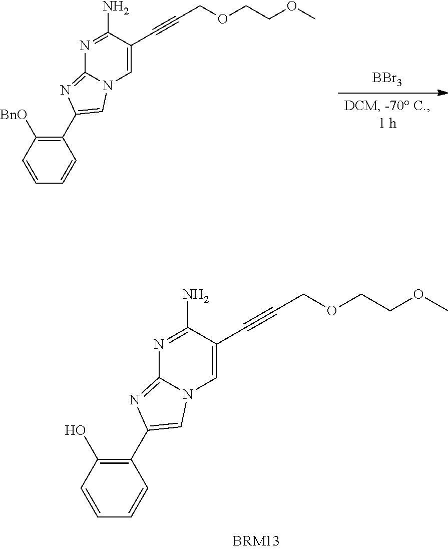 [SMILES: COCCOCC#Cc1cn2cc(-c3ccccc3OCc3ccccc3)nc2nc1N ; "BBr3" ; "DCM, -70° C.,1 h" ; COCCOCC#Cc1cn2cc(-c3ccccc3O)nc2nc1N]

### Exemplary Synthesis of Compound BRM14

### Step 1

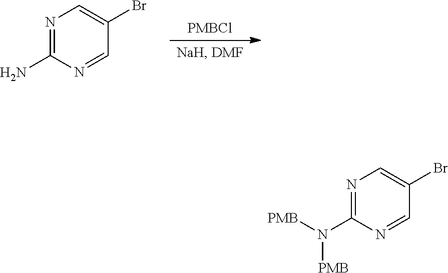

To the solution of 5-bromopyrimidin-2-amine (3.0 g, 17.24 mmol, 1.0 eq) in DMF (30 mL) was added NaH (1.72 g, 60% dispersion in mineral oil, 43.10 mmol, 2.5 eq) at 0° C. The mixture was stirred at 0° C. for 0.5 hour. PMB-Cl (5.94 g, 37.93 mmol, 5.17 mL, 2.2 eq) was added. The mixture was warmed to 25° C. and stirred for 16 hours. The mixture was poured into water (50 mL). The mixture was extracted with ethyl acetate (80 mL×3), the combined organic layers were washed with brine (60 mL), dried over Na₂SO₄, filtered, and concentrated. The residue was purified by flash chromatography (petroleum ether/ethyl acetate=50/1 to 3/1) to afford 5-bromo-N,N-bis(4-methoxybenzyl) pyrimidin-2-amine (6.2 g, 14.97 mmol) as a white solid.

### Step 2

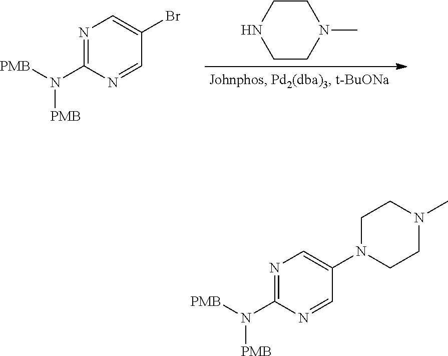

To a solution of 5-bromo-N,N-bis(4-methoxybenzyl)pyrimidin-2-amine (2.0 g, 4.83 mmol, 1.0 eq) and 1-methylpiperazine (967 mg, 9.65 mmol, 1.07 mL, 2.0 eq) in toluene (30 mL) was added NaOtBu (927.84 mg, 9.65 mmol, 2.0 eq), JohnPhos [(2-biphenyl)di-tert-butylphosphine] (144 mg, 483 umol, 0.10 eq) and Pd₂(dba)₃ (221 mg, 242 umol, 0.05 eq). The mixture was heated to 85° C. and stirred for 30 hours under N2 in sealed tube. The mixture was filtered, and the cake was washed with ethyl acetate (15 mL×4). The combined organic layer was washed with brine (30 mL), dried over Na₂SO₄, filtered and concentrated. The residue was purified by flash chromatography (petroleum ether/ethyl acetate=30/1, then dichloromethane/methanol=20/1) to afford N,N-bis(4-methoxybenzyl)-5-(4-methylpiperazin-1-yl)pyrimidin-2-amine (1.02 g, 2.35 mmol) as a white solid.

### Step 3

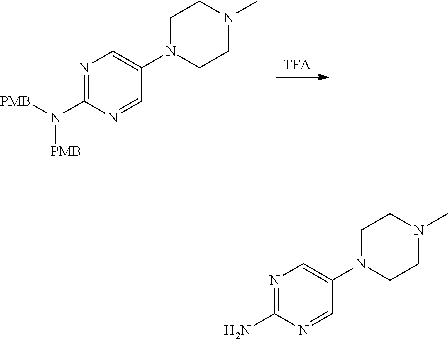

A solution of N,N-bis(4-methoxybenzyl)-5-(4-methylpiperazin-1-yl)pyrimidin-2-amine (600 mg, 1.38 mmol) in TFA (5 mL) was stirred at 25° C. for 5 hr. Water (10 mL) was added. The mixture was filtered, and the filtrate was extracted with ethyl acetate (10 mL×2). The pH of the aqueous layer was adjusted to 9 with saturated Na₂CO₃. Then the aqueous layer was extracted with dichloromethane (20 mL×5). The organic layer was dried over Na₂SO₄, filtered and concentrated to afford crude 5-(4-methylpiperazin-1-yl)pyrimidin-2-amine (200 mg) as a yellow oil.

### Step 4

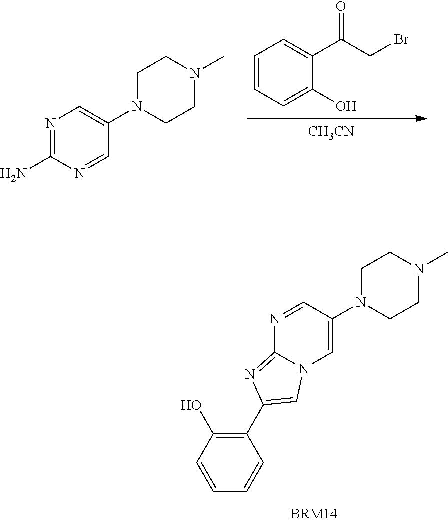

The solution of 5-(4-methylpiperazin-1-yl)pyrimidin-2-amine (200 mg, 1.03 mmol, 1.0 eq), DMAP (6.32 mg, 51.75 umol, 0.05 eq) and 2-bromo-1-(2-hydroxyphenyl)ethanone (223 mg, 1.03 mmol, 1 eq) in CH₃CN (8 mL) was heated to 85° C. and stirred for 40 hr in sealed tube. The mixture was filtered, and the cake was washed with EA (3 mL×3). The cake was dried to give the crude product. The crude product was purified by preparative HPLC (Phenomenex Luna C18 100*30 mm*5 um; mobile phase: [water (0.1% TFA)-MeCN]; B %: 0%-15%, 9 min) to afford 2-[6-(4-methylpiperazin-1-yl)imidazo[1,2-a]pyrimidin-2-yl]phenol (10.3 mg, 32.83 umol) as a gray solid.

### Exemplary Synthesis of Compound BRM15

2-(2-methoxyphenyl)-6-(3-methyl-3,8-diazabicyclo[3.2.1]octan-8-yl)imidazo[1,2-a]pyrimidine was prepared according to the scheme below using procedures described for other examples herein (BRM10 and BRM14), as well as procedures generally known to those skilled in the art.

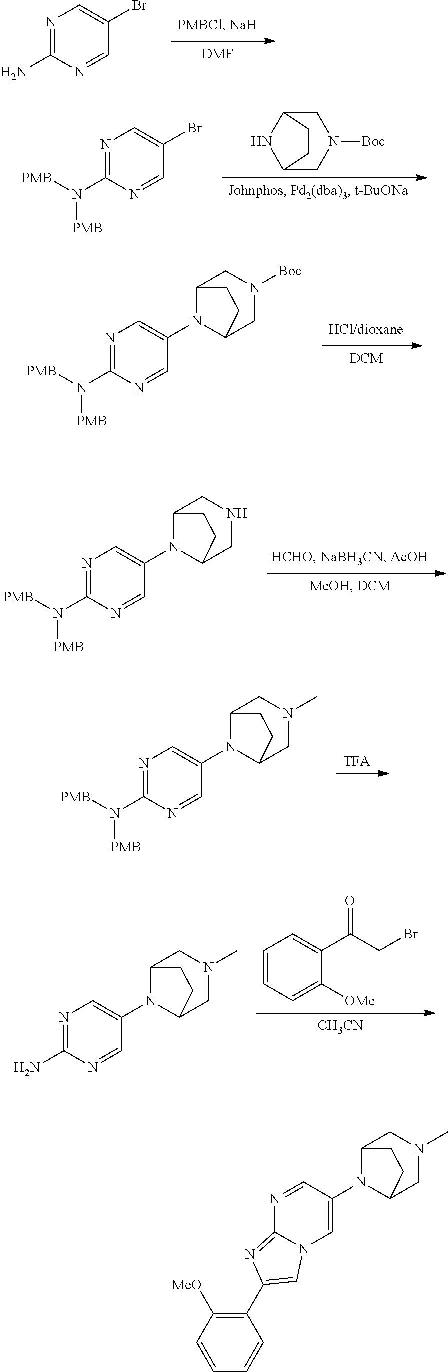

### Step 1

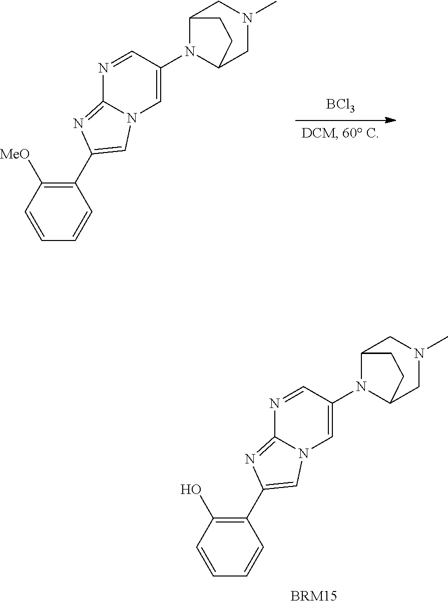

The mixture of 2-(2-methoxyphenyl)-6-(3-methyl-3,8-diazabicyclo[3.2.1]octan-8-yl) imidazo[1,2-a]pyrimidine (64 mg, 183.16 umol, 1.0 eq) and BCl₃ (3.60 mL of 1M solution in DCM)) was heated to 60° C. and stirred for 24 hours. The mixture was quenched by water (10 mL) at 0° C. The pH of the mixture was adjusted to 9 by adding sat. Na₂CO₃ solution. The mixture was extracted with dichloromethane (15 mL×4). The combined organic layer was washed with brine (20 mL), dried over Na₂SO₄, filtered and concentrated. The residue was purified by preparative HPLC (Phenomenex luna C18 100*40 mm*3 um; mobile phase: [water (0.225% formic acid)-acetonitrile]; B %: 10%-40%, 10 min) to afford 2-[6-(3-methyl-3,8-diazabicyclo[3.2.1]octan-8-yl)imidazo[1,2-a]pyrimidin-2-yl] phenol (10.2 mg, 30.26 umol) as a yellow solid.

### Exemplary Synthesis of Compound BRM16

### Step 1

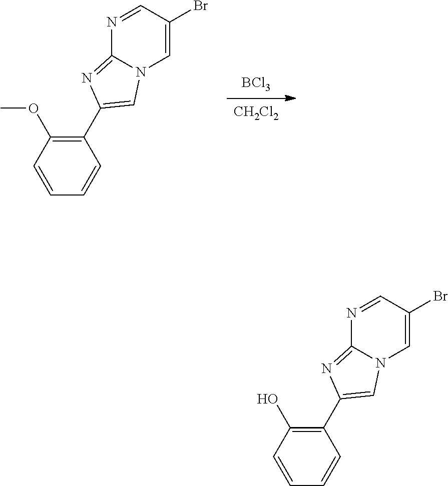 [SMILES: COc1ccccc1-c1cn2cc(Br)cnc2n1 ; "BCl3" ; "CH2Cl2" ; Oc1ccccc1-c1cn2cc(Br)cnc2n1]

6-Bromo-2-(2-methoxyphenyl)imidazo[1,2-a]pyrimidine (150 mg, 493.2 umol, 1.0 eq) in BCl₃ (1 M in DCM, 3.0 mL, 6.08 eq) was stirred at 60° C. for 12 hours. The mixture was poured into saturated NaHCO₃ solution (50 mL) at 0° C. The aqueous phase was extracted with dichloromethane (50 mL×3). The combined organic phase was dried with anhydrous Na₂SO₄, filtered, and concentrated over vacuum. The residue was purified by silica gel chromatography (ethyl acetate) to afford 2-(6-bromoimidazo[1,2-a]pyrimidin-2-yl)phenol (140 mg, 483 umol) as a yellow solid.

### Step 2

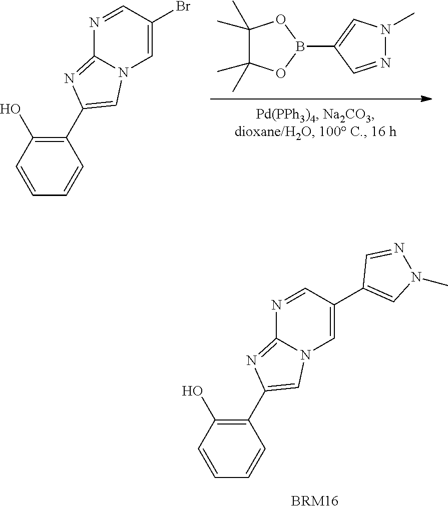

To a mixture of 2-(6-bromoimidazo[1,2-a]pyrimidin-2-yl)phenol (60 mg, 206.8 umol, 1.0 eq) and 1-methylpyrazole-4-boronic acid pinacol ester (64.6 mg, 310.2 umol, 1.5 eq) in dioxane (4.0 mL) and H₂O (1.0 mL) was added Na₂CO₃ (65.8 mg, 620.44 umol, 3.0 eq) and Pd(PPh₃)₄ (23.9 mg, 20.68 umol, 0.1 eq) in one portion under N2. The mixture was stirred at 100° C. for 12 hours. The residue was purified by preparative HPLC (Phenomenex luna C18 100*40 mm*3 um, water (0.225% formic acid)-acetonitrile) to afford 2-[6-(1-methylpyrazol-4-yl)imidazo[1,2-a]pyrimidin-2-yl] phenol (9.4 mg, 31.30 umol) as a yellow solid.

### Exemplary Synthesis of Compound BRM17

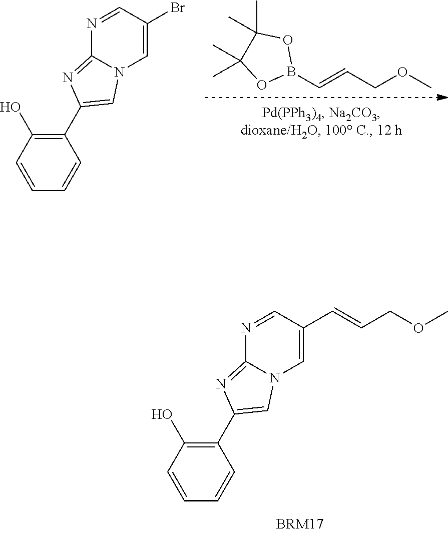

To a mixture of 2-(6-bromoimidazo[1,2-a]pyrimidin-2-yl)phenol (60 mg, 206.8 umol, 1.0 eq) and 2-[(E)-3-methoxyprop-1-enyl]boronic acid pinacol ester (61.4 mg, 310.2 umol, 1.5 eq) in dioxane (4.0 mL) and water (1.0 mL) was added Na₂CO₃ (65.8 mg, 620.4 umol, 3.0 eq) and Pd(PPh₃)₄ (23.9 mg, 20.68 umol, 0.1 eq) in one portion under N2. The mixture was stirred at 100° C. for 12 hours. The mixture was filtered and concentrated under vacuum. The residue was purified by preparative HPLC (Phenomenex luna C18 100*40 mm*3 um, water (0.225% formic acid)-acetonitrile) to afford 2-[6-[(E)-3-methoxyprop-1-enyl]imidazo[1,2-a]pyrimidin-2-yl]phenol (8.9 mg, 31.64 umol) as a yellow solid.

### Exemplary Synthesis of Compound BRM18

### Step 1

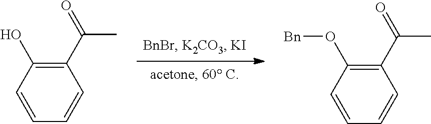

To a mixture of 1-(2-hydroxyphenyl)ethanone (5.0 g, 36.72 mmol, 1.0 eq), K₂CO₃ (10.2 g, 73.45 mmol, 2.0 eq) and KI (6.1 g, 36.72 mmol, 1.0 eq) in acetonitrile (100 mL) was added BnBr (12.6 g, 73.45 mmol, 2.0 eq). The mixture was stirred at 70° C. for 16 hours under N₂. The mixture was filtered, the filter cake was washed with ethyl acetate (30 mL×2). The combined organic layer was concentrated. The crude product was purified by Biotage® combi flash (Column: 80 g Biotage® Silica Flash column; Eluent: gradient 0-3% ethyl acetate in petroleum ether; Gradient time: 35 min; Hold time: 20 min; Flow rate: 100 mL/min). Pure fractions were combined and concentrated under reduced pressure to afford 1-(2-benzyloxyphenyl)ethanone (7.1 g, 31.19 mmol) as a yellow oil.

### Step 2

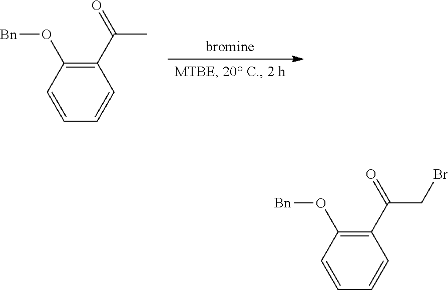 [SMILES: CC(=O)c1ccccc1OCc1ccccc1 ; "MTBE, 20° C., 2 h" ; "bromine" ; O=C(CBr)c1ccccc1OCc1ccccc1]

To a solution of 1-(2-benzyloxyphenyl)ethanone (7.1 g, 31.38 mmol, 1.0 eq) in methyl tert-butyl ether (200 mL) was added Br₂ (5 g, 31.38 mmol, 1.0 eq) dropwise at 20° C. over 5 minutes. Then the mixture was stirred at 20° C. for 2 hours. The mixture was washed with sat. aq. NaHCO₃ (150 mL×2), water (100 mL×2) and brine (100 mL). Then the organic phase was dried over Na₂SO₄, filtered, and concentrated to give the product 1-(2-benzyloxyphenyl)-2-bromo-ethanone (9.4 g, 25.04 mmol) as an off-white solid which was used in the next step without purification.

### Step 3

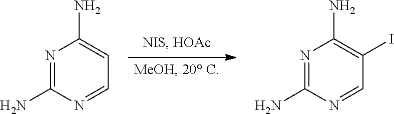

To a solution of pyrimidine-2,4-diamine (4.5 g, 40.87 mmol, 1.0 eq) in AcOH (80 mL) and methanol (80 mL) was added NIS (9.3 g, 41.07 mmol, 1.01 eq) at 0° C. The mixture was stirred at 20° C. for 2 hours. The mixture was quenched by sat. aq. Na₂S₂O₃ at 0° C., and pH of the mixture was adjusted to 7 with sat. aq. NaOH. Then the mixture was filtered, and the filter cake was washed with water (30 mL×3) to give crude product 5-iodopyrimidine-2,4-diamine (4.8 g, 20.24 mmol) as a light yellow solid which was used in the next step without purification.

### Step 4

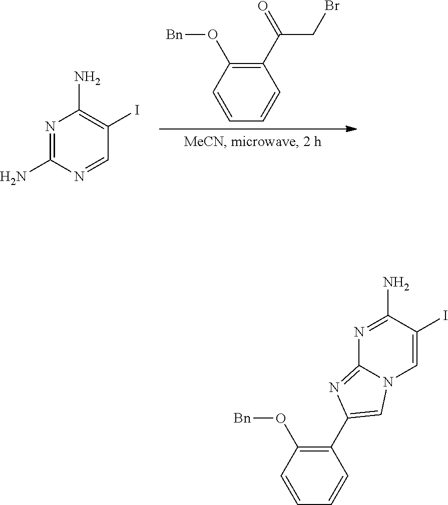

A mixture of 5-iodopyrimidine-2,4-diamine (671 mg, 2.84 mmol, 1.0 eq), 1-(2-benzyloxyphenyl)-2-bromo-ethanone (954 mg, 3.13 mmol, 1.1 eq) and EtOH (12 mL) in a sealed microwave tube was microwave irradiated at 80° C. for 2 hours. The mixture was concentrated, and the crude product was purified by Biotage® combi flash (Column: 10 g Biotage® Silica Flash column; Eluent: gradient 0-8% Methanol in Dichloromethane; Gradient time: 60 min; Hold time: 60 min; Flow rate: 40 mL/min). Pure fractions were combined and concentrated under reduced pressure to afford 2-(2-benzyloxyphenyl)-6-iodo-imidazo[1,2-a]pyrimidin-7-amine (650 mg, 1.46 mmol) as an off-white solid.

### Step 5

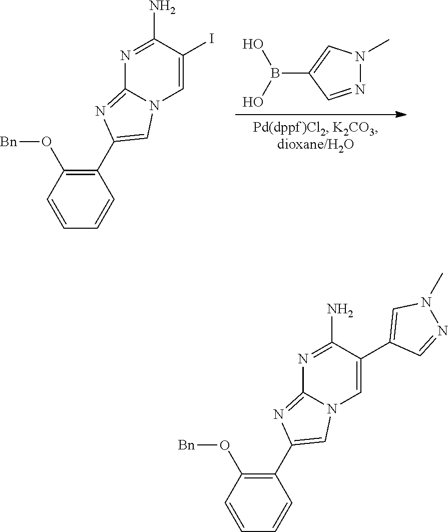

To a solution of 2-(2-benzyloxyphenyl)-6-iodo-imidazo[1,2-a]pyrimidin-7-amine (100 mg, 226.12 umol, 1.0 eq) and 1-methyl-4-(4,4,5,5-tetramethyl-1,3,2-dioxaborolan-2-yl)pyrazole (71 mg, 339.17 umol, 1.5 eq) in dioxane (3.0 mL) and H₂O (0.5 mL) was added K₂CO₃ (47 mg, 339.17 umol, 1.5 eq) and Pd(dppf)Cl₂ (17 mg, 22.61 umol, 0.1 eq). The mixture was stirred at 100° C. for 16 hours under N₂. The mixture was concentrated to give the crude product. The crude product was purified by Biotage® combi flash (Column: 4 g Biotage® Silica Flash column; Eluent: gradient 0-2% methanol in dichlormethane; Gradient time: 15 min; Hold time: 20 min; Flow rate: 18 mL/min). Pure fractions were combined and concentrated under reduced pressure to afford 2-(2-benzyloxyphenyl)-6-(1-methylpyrazol-4-yl)imidazo[1,2-a]pyrimidin-7-amine (60 mg, 150.89 umol) as a light yellow solid.

### Step 6

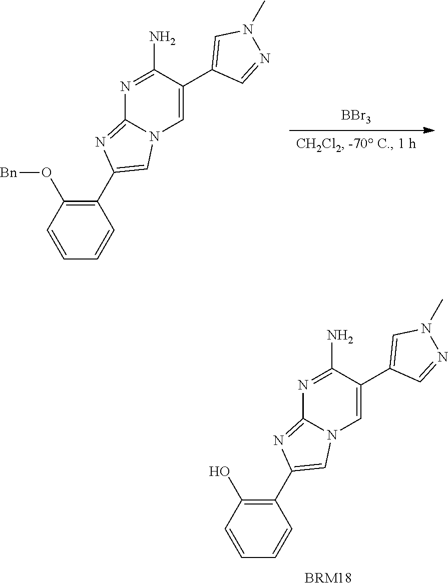

To a mixture of 2-(2-benzyloxyphenyl)-6-(1-methylpyrazol-4-yl)imidazo[1,2-a]pyrimidin-7-amine (60 mg, 151.35 umol, 1.0 eq) in dichloromethane (2.0 mL) was added BBr₃ (76 mg, 302.69 umol, 2.0 eq) at −70° C. under N₂. The mixture was stirred at −70° C. for 1 hour under N₂. The mixture was quenched with methanol (3.0 mL) at −70° C., and pH of the mixture was adjusted to 7-8 with aq. NH₄OH. The mixture was then concentrated under reduced pressure at 40° C. The crude product was purified by prep-HPLC (Column: Phenomenex Gemini-NX C18 75*30 mm*3 um; Eluent: gradient 10-36% water (0.04% HCl) in acetonitrile; Gradient time: 20 min; Hold time: 2 min; Flow rate: 25 mL/min) to afford 2-[7-amino-6-(1-methylpyrazol-4-yl)imidazo[1,2-a]pyrimidin-2-yl]phenol (16.5 mg, 53.70 umol) as a white solid.

### Exemplary Synthesis of Compound BRM19

### Step 1

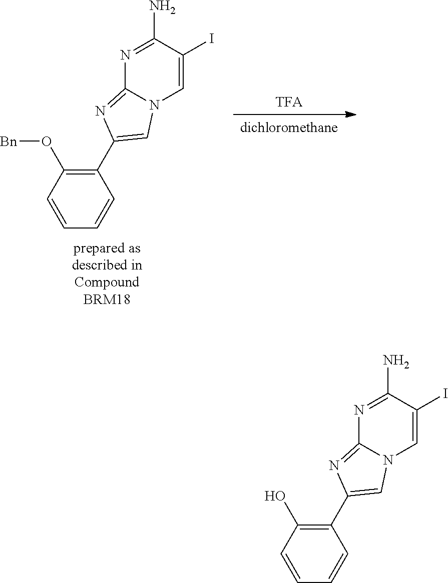 [SMILES: Nc1nc2nc(-c3ccccc3OCc3ccccc3)cn2cc1I ; "dichloromethane" ; "prepared asdescribed inCompoundBRM18" ; Nc1nc2nc(-c3ccccc3O)cn2cc1I ; "TFA"]

To a mixture of 2-(2-benzyloxyphenyl)-6-iodo-imidazo[1,2-a]pyrimidin-7-amine (200 mg, 452.2 umol, 1.0 eq) in dichloromethane (5.0 mL) was added TFA (3.0 mL). The mixture was stirred at 20° C. for 2 hours. When LCMS showed only partial conversion, the mixture was concentrated under reduced pressure, TFA (5.0 mL) was added, and the mixture was stirred at 50° C. for an additional 5 hours (LCMS indicated completion of the reaction), and then concentrated to give crude 2-(7-amino-6-iodo-imidazo[1,2-a]pyrimidin-2-yl)phenol (260 mg) as a yellow solid, which was used in the next step without purification.

### Step 2

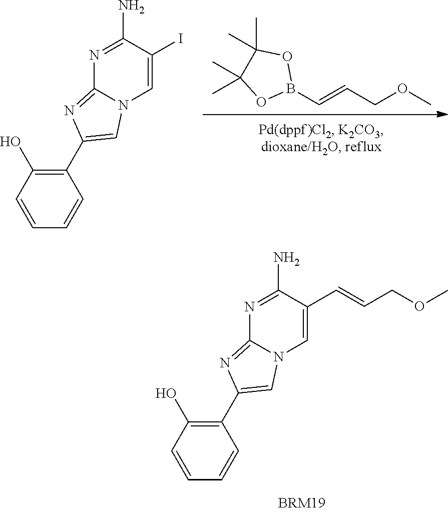

To a solution of 2-(7-amino-6-iodo-imidazo[1,2-a]pyrimidin-2-yl)phenol (260 mg, 465.2 umol, 1.0 eq) and 2-[(E)-3-methoxyprop-1-enyl]boronic acid pinacol ester (138 mg, 697.8 umol, 1.5 eq) in dioxane (3.0 mL) and water (1.0 mL) were added Pd(dppf)Cl₂ (34 mg, 46.52 umol, 0.1 eq) and K₂CO₃ (161 mg, 1.16 mmol, 2.5 eq). The mixture was stirred at 100° C. for 16 hours under nitrogen atmosphere. The mixture was filtered and concentrated under vacuum. The crude product was purified by preparative HPLC (Column: Waters Xbridge 150*25 mm*5 um; Eluent: gradient 40-70% water (0.05% NH₃H₂O+10 mM NH₄HCO₃) in acetonitrile; Gradient time: 10 min; Hold time: 1 min; Flow rate: 25 mL/min). Pure fractions were combined and concentrated under reduced pressure, then lyophilized to afford 2-[7-amino-6-[(E)-3-methoxyprop-1-enyl]imidazo [1,2-a]pyrimidin-2-yl]phenol (10.5 mg, 35.22 umol) as a yellow solid.

### Exemplary Synthesis of Compound 29

### Step 1

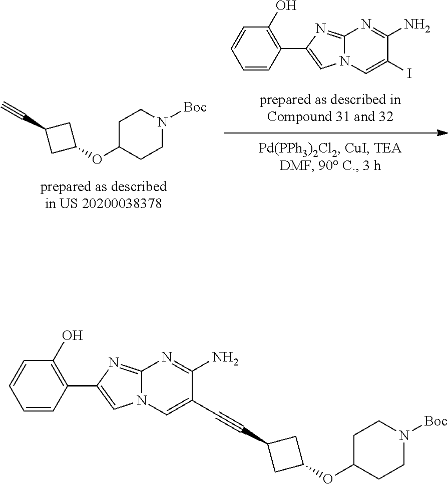 [SMILES: Nc1nc2nc(-c3ccccc3O)cn2cc1I ; C#C[C@H]1C[C@H](OC2CCN(C(=O)OC(C)(C)C)CC2)C1 ; CC(C)(C)OC(=O)N1CCC(O[C@H]2C[C@H](C#Cc3cn4cc(-c5ccccc5O)nc4nc3N)C2)CC1]

To a mixture of 2-(7-amino-6-iodo-imidazo[1,2-a]pyrimidin-2-yl)phenol (82 mg, 0.23 mmol, 1 eq), bis(triphenylphosphine)palladium(II) dichloride (25 mg, 0.034 mmol, 0.15 eq), cuprous iodide (9 mg, 0.05 mmol, 0.2 eq) and tert-butyl 4-(3-ethynylcyclobutoxy)piperidine-1-carboxylate (78 mg, 0.28 mmol, 1.2 eq) [prepared as described in US 20200038378] in N,N-dimethylformamide (2 mL) was added triethylamine (70 mg, 0.69 mmol, 3 eq). The mixture was stirred at 90° C. for 3 hours. The mixture was cooled to 20° C. and concentrated under reduced pressure at 45° C. The residue was purified by semi-preparative reverse phase HPLC (column: Phenomenex Gemini-NX C18 75*30 mm*3 um; mobile phase: [water (0.1% TFA)-ACN]; B %: 45%-55%, 7 min). Compound tert-butyl 4-[3-[2-[7-amino-2-(2-hydroxyphenyl)imidazo[1,2-a]pyrimidin-6-yl]ethynyl]cyclobutoxy]piperidine-1-carboxylate (90 mg, 0.18 mmol) was obtained as a yellow solid.

### Step 2

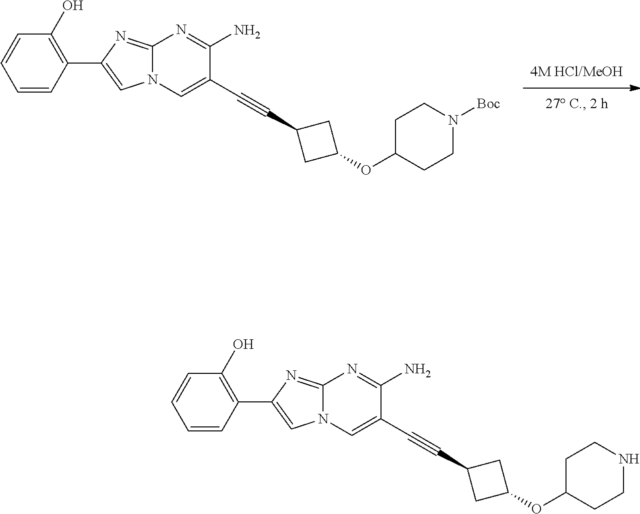 [SMILES: CC(C)(C)OC(=O)N1CCC(O[C@H]2C[C@H](C#Cc3cn4cc(-c5ccccc5O)nc4nc3N)C2)CC1 ; Nc1nc2nc(-c3ccccc3O)cn2cc1C#C[C@H]1C[C@H](OC2CCNCC2)C1]

A solution of tert-butyl 4-[3-[2-[7-amino-2-(2-hydroxyphenyl)imidazo[1,2-a]pyrimidin-6-yl] ethynyl]cyclobutoxy]piperidine-1-carboxylate (86 mg, 0.17 mmol, 1 eq) in 4M HCl in MeOH (3 mL) was stirred at 27° C. for 2 hours. The mixture was concentrated under reduced pressure at 45° C. The crude product 2-[7-amino-6-[2-[3-(4-piperidyloxy)cyclobutyl]ethynyl]imidazo[1,2-a]pyrimidin-2-yl] phenol hydrochloride (76 mg) was obtained as an off-white solid.

### Step 3

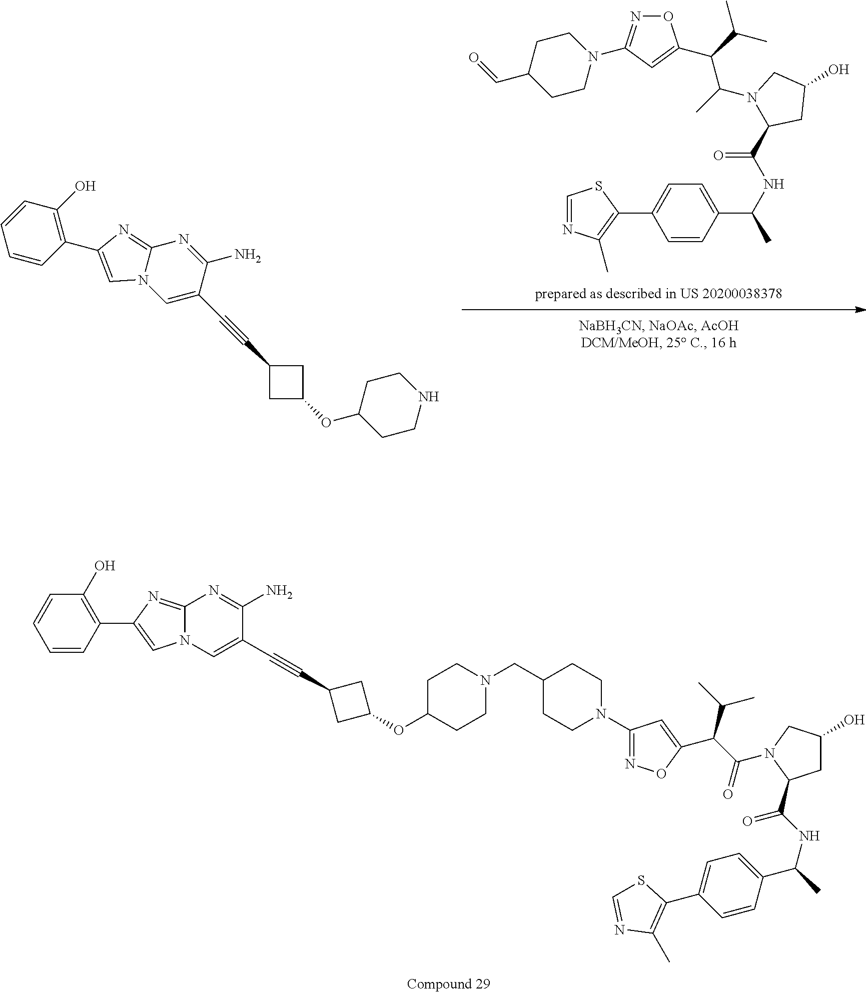 [SMILES: Nc1nc2nc(-c3ccccc3O)cn2cc1C#C[C@H]1C[C@H](OC2CCNCC2)C1 ; Cc1ncsc1-c1ccc([C@H](C)NC(=O)[C@@H]2C[C@@H](O)CN2C(C)[C@@H](c2cc(N3CCC(C=O)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](C#Cc6cn7cc(-c8ccccc8O)nc7nc6N)C5)CC4)CC3)no2)C(C)C)cc1]

To a mixture of 2-[7-amino-6-[2-[3-(4-piperidyloxy)cyclobutyl]ethynyl]imidazo[1,2-a]pyrimidin-2-yl]phenol (76 mg, 0.17 mmol, 1 eq, hydrochloride), sodium acetate (28 mg, 0.35 mmol, 2 eq), acetic acid (10 mg, 0.17 mmol, 1 eq) and (2S,4R)-1-[(2R)-2-[3-(4-formyl-1-piperidyl)230soxazole-5-yl]-3-methyl-butanoyl]-4-hydroxy-N-[(1S)-1-[4-(4-methylthiazol-5-yl)phenyl]ethyl]pyrrolidine-2-carboxamide (103 mg, 0.17 mmol, 1 eq) [prepared as described in US 20200038378] in dichloromethane (2 mL) and methanol (4 mL) was added sodium cyanoborohydride (54 mg, 0.86 mmol, 5 eq). The mixture was stirred at 25° C. for 16 hours. The mixture was concentrated under reduced pressure at 45° C. The residue was purified by semi-preparative reverse phase HPLC (column: Phenomenex Gemini-NX C18 75*30 mm*3 um; mobile phase: [water (0.225% FA)-ACN]; B %: 18%-48%, 7 min). Compound (2S,4R)-1-[(2R)-2-[3-[4-[[4-[3-[2-[7-amino-2-(2-hydroxyphenyl)imidazo[1,2-a]pyrimidin-6-yl]ethynyl]cyclobutoxy]-1-piperidyl]methyl]-1-piperidyl]230soxazole-5-yl]-3-methyl-butanoyl]-4-hydroxy-N-[(1S)-1-[4-(4-methylthiazol-5-yl)phenyl]ethyl]pyrrolidine-2-carboxamide (61 mg, 0.06 mmol) was obtained as a yellow solid.

### Exemplary Synthesis of Compound 30

### Step 1

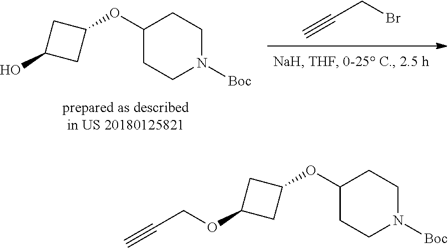

To a solution of tert-butyl 4-(3-hydroxycyclobutoxy)piperidine-1-carboxylate (260 mg, 0.96 mmol, 1 eq) in tetrahydrofuran (5 mL) was added sodium hydride (57 mg, 1.44 mmol, 60% in mineral oil, 1.5 eq) at 0° C. After the mixture was stirred at 0° C. for 0.5 hours, 3-bromoprop-1-yne (214 mg, 1.44 mmol, 1.5 eq) was added to the mixture. The reaction mixture was stirred at 25° C. for 2 hours. The reaction mixture was quenched with saturated ammonium chloride solution (30 mL) and extracted with ethyl acetate (30 mL×2). The combined organic layer was dried over anhydrous sodium sulfate, filtered, and concentrated under vacuum. The residue was purified by silica gel column chromatography (petroleum ether/ethyl acetate=10/1). Tert-Butyl 4-(3-prop-2-ynoxycyclobutoxy) piperidine-1-carboxylate (170 mg, 0.55 mmol) was obtained as a colorless gum.

Tert-Butyl 4-(3-prop-2-ynoxycyclobutoxy) piperidine-1-carboxylate was converted to the title compound according to the scheme below using procedures described for Compound 28.

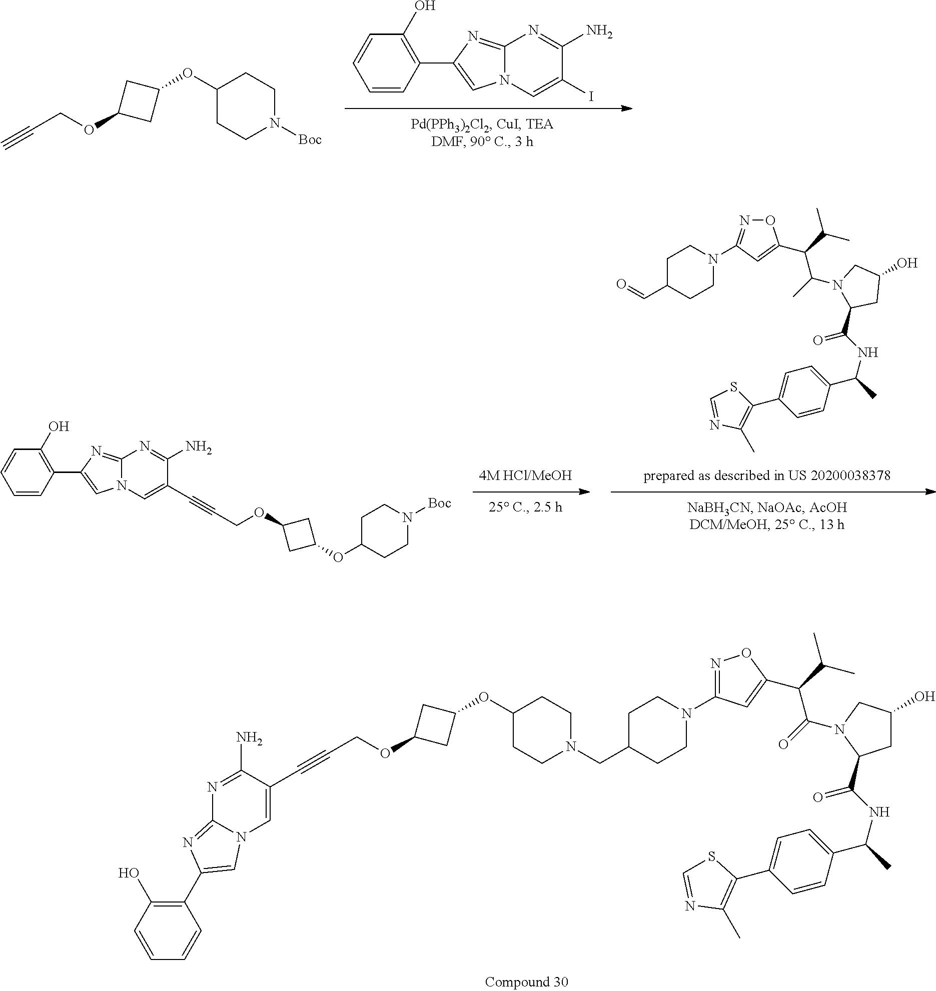 [SMILES: C#CCO[C@H]1C[C@H](OC2CCN(C(=O)OC(C)(C)C)CC2)C1 ; Nc1nc2nc(-c3ccccc3O)cn2cc1I ; CC(C)(C)OC(=O)N1CCC(O[C@H]2C[C@H](OCC#Cc3cn4cc(-c5ccccc5O)nc4nc3N)C2)CC1 ; Cc1ncsc1-c1ccc([C@H](C)NC(=O)[C@@H]2C[C@@H](O)CN2C(C)[C@@H](c2cc(N3CCC(C=O)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](OCC#Cc6cn7cc(-c8ccccc8O)nc7nc6N)C5)CC4)CC3)no2)C(C)C)cc1]

### Exemplary Synthesis of Compounds 31 and 32

### Step 1

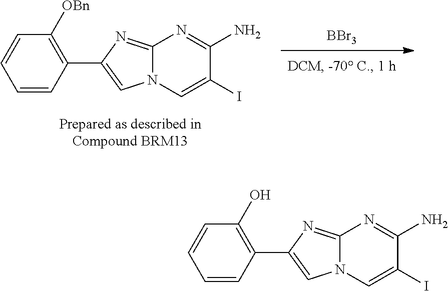 [SMILES: Nc1nc2nc(-c3ccccc3OCc3ccccc3)cn2cc1I ; "BBr3" ; Nc1nc2nc(-c3ccccc3O)cn2cc1I]

To a solution of 2-(2-benzyloxyphenyl)-6-iodo-imidazo[1,2-a]pyrimidin-7-amine (420 mg, 0.94 mmol, 1 eq) in dichloromethane (40 mL) was added boron tribromide (476 mg, 1.90 mmol, 0.18 mL, 2 eq) in one portion at −70° C. under nitrogen. The mixture was stirred at −70° C. for 1 hour. The mixture was quenched by methanol (3 mL) at −70° C. and concentrated under reduced pressure at 50° C. The residue was triturated with ethyl acetate (10 mL) and filtered, and the cake was collected by filtration and dried under vacuum. Compound 2-(7-amino-6-iodo-imidazo[1,2-a]pyrimidin-2-yl)phenol hydrobromide (380 mg, 0.87 mmol) was obtained as a white solid.

### Step 2

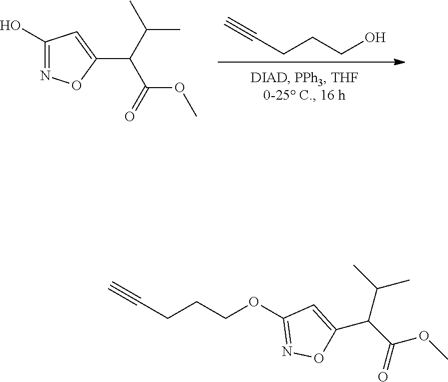

To a mixture of methyl 2-(3-hydroxyisoxazol-5-yl)-3-methyl-butanoate (0.7 g, 3.51 mmol, 1 eq), triphenylphosphine (1.38 g, 5.27 mmol, 1.5 eq) and pent-4-yn-1-ol (443.38 mg, 5.27 mmol, 1.5 eq) in tetrahydrofuran (8 mL) was added diisopropylazodicarboxylate (1.07 g, 5.27 mmol, 1.02 mL, 1.5 eq) dropwise at 0° C. under nitrogen. The mixture was stirred at 25° C. for 16 hours. The mixture was poured into ice-water (w/w=1/1, 30 mL) and stirred for 5 minutes. The aqueous phase was extracted with ethyl acetate (20 mL×3). The combined organic phase was washed with brine (15 mL×2), dried with anhydrous sodium sulfate, filtered, and concentrated in vacuum. The residue was purified by semi-preparative reverse phase HPLC (column: Phenomenex luna C18 150*40 mm*15 um; mobile phase: [water (0.1% TFA)-ACN]; B %: 42%-72%, 10 min). Compound methyl 3-methyl-2-(3-pent-4-ynoxyisoxazol-5-yl)butanoate (690 mg, 2.60 mmol) was obtained as a yellow oil.

### Step 3

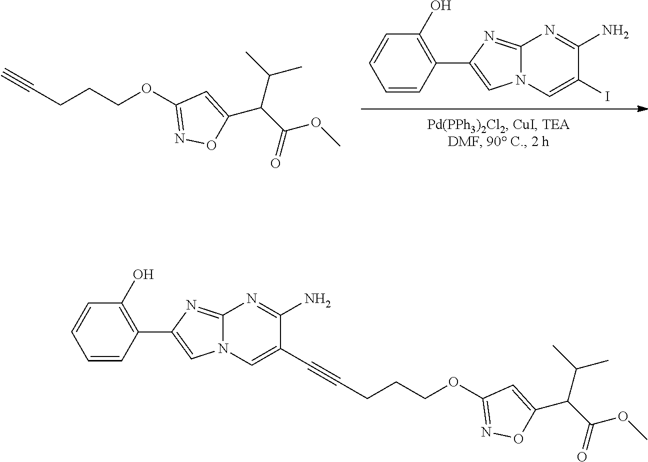 [SMILES: C#CCCCOc1cc(C(C(=O)OC)C(C)C)on1 ; Nc1nc2nc(-c3ccccc3O)cn2cc1I ; COC(=O)C(c1cc(OCCCC#Cc2cn3cc(-c4ccccc4O)nc3nc2N)no1)C(C)C]

To a mixture of 2-(7-amino-6-iodo-imidazo[1,2-a]pyrimidin-2-yl)phenol (0.15 g, 0.42 mmol, 1 eq), cuprous iodide (16 mg, 0.08 mmol, 0.2 eq), triethylamine (129 mg, 1.28 mmol, 3 eq) and methyl 3-methyl-2-(3-pent-4-ynoxyisoxazol-5-yl)butanoate (169 mg, 0.6 mmol, 1.5 eq) in N,N-dimethylformamide (4 mL) was added bis(triphenylphosphine)palladium(ii) dichloride (30 mg, 0.04 mmol, 0.1 eq) in one portion at 0° C. under nitrogen. The mixture was stirred at 90° C. for 2 hours. The mixture was cooled to 20° C. and concentrated under reduced pressure at 45° C. The residue was purified by semi-preparative reverse phase HPLC (column: Phenomenex Gemini-NX C18 75*30 mm*3 um; mobile phase: [water (0.1% TFA)-ACN]; B %: 32%-42%, 7 min). Compound methyl 2-[3-[5-[7-amino-2-(2-hydroxyphenyl)imidazo[1,2-a]pyrimidin-6-yl]pent-4-ynoxy]isoxazole-5-yl]-3-methyl-butanoate (152 mg, 0.31 mmol) was obtained as a white solid.

### Step 4

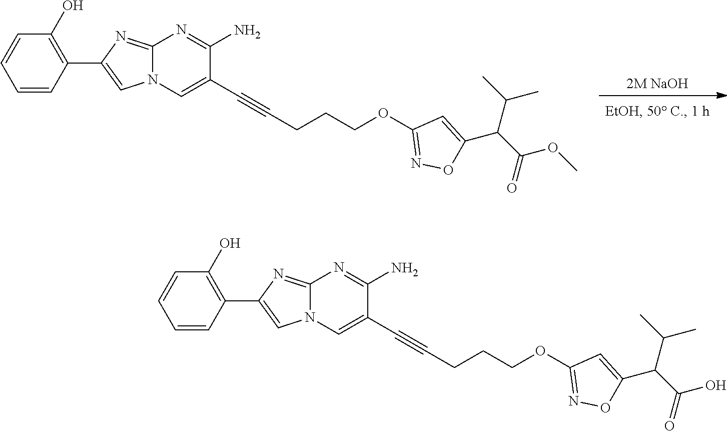 [SMILES: COC(=O)C(c1cc(OCCCC#Cc2cn3cc(-c4ccccc4O)nc3nc2N)no1)C(C)C ; "2M NaOH" ; CC(C)C(C(=O)O)c1cc(OCCCC#Cc2cn3cc(-c4ccccc4O)nc3nc2N)no1]

To a mixture of methyl 2-[3-[5-[7-amino-2-(2-hydroxyphenyl)imidazo[1,2-a]pyrimidin-6-yl] pent-4-ynoxy]232soxazole-5-yl]-3-methyl-butanoate (150 mg, 0.30 mmol, 1 eq) in ethanol (4 mL) was added 2N sodium hydroxide solution in water (2 mL, 13.05 eq). The mixture was stirred at 50° C. for 1 hour. The mixture was cooled to 10° C., pH was adjusted to 5 with 1 M HCl, and the mixture was concentrated under reduced pressure at 45° C. The residue was purified by semi-preparative reverse phase HPLC (column: Phenomenex Gemini-NX C18 75*30 mm*3 um; mobile phase: [water (0.1% TFA)-ACN]; B %: 25%-35%, 7 min). Compound 2-[3-[5-[7-amino-2-(2-hydroxyphenyl)imidazo[1,2-a]pyrimidin-6-yl]pent-4-ynoxy]isoxazole-5-yl]-3-methyl-butanoic acid (60 mg, 0.12 mmol) was obtained as a white solid.

### Step 5

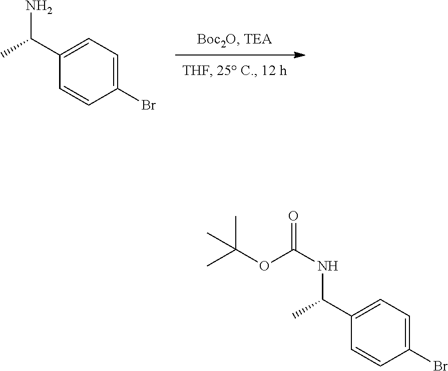

To a solution of (1S)-1-(4-bromophenyl)ethanamine (24.9 g, 124.5 mmol, 1 eq) in tetrahydrofuran (350 mL) was added triethylamine (37.8 g, 373.4 mmol, 3 eq) followed by di-tert-butyl dicarbonate (28.5 g, 130.7 mmol, 30 mL, 1.05 eq) dropwise at 0° C. under nitrogen. The mixture was then stirred at 25° C. for 12 hours. The reaction mixture was concentrated under reduced pressure to remove tetrahydrofuran. Water (400 mL) was added, and the mixture was stirred for 1 minute. The aqueous phase was extracted with ethyl acetate (200 mL×3). The combined organic phase was washed with brine (200 mL×2), dried over anhydrous sodium sulfate, filtered, and concentrated in vacuum. The crude product was triturated with petroleum ether (250 mL). Compound tert-butyl N-[(1S)-1-(4-bromophenyl)ethyl]carbamate (34.5 g, 114.93 mmol, 92% yield) was obtained as a white solid.

### Step 6

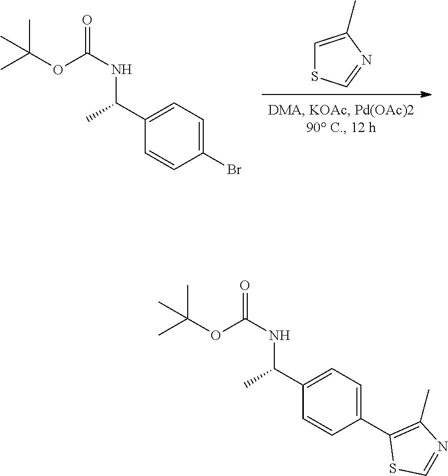

To a solution of tert-butyl N-[(1S)-1-(4-bromophenyl)ethyl]carbamate (14.5 g, 48.30 mmol, 1 eq) and 4-methylthiazole (7.18 g, 72.45 mmol, 1.5 eq) in dimethylacetamide (15 mL) was added palladium(II) acetate (542 mg, 2.42 mmol, 0.05 eq) and potassium acetate (9.48 g, 96.61 mmol, 2 eq). The mixture was stirred at 90° C. for 12 hours. Water (300 mL) was added, and the mixture was stirred for 1 minute. The aqueous phase was extracted with ethyl acetate (100 mL×3). The combined organic phase was washed with brine (100 mL×2), dried with anhydrous sodium sulfate, filtered, and concentrated in vacuum. The residue was purified by reverse phase C18 column chromatography [ACN/H2O (0.5% FA) from 5% to 50%]. tert-Butyl N-[(1S)-1-[4-(4-methylthiazol-5-yl)phenyl]ethyl]carbamate (9.8 g, 29.85 mmol, 61% yield) was obtained as a gray solid.

### Step 7

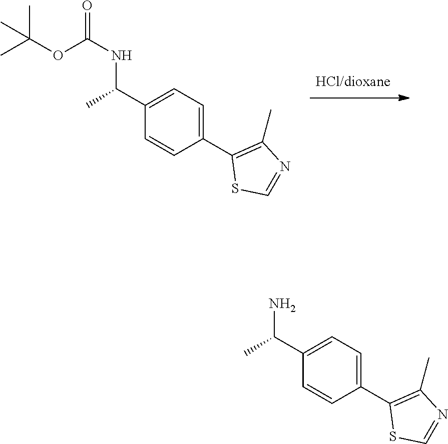 [SMILES: Cc1ncsc1-c1ccc([C@H](C)NC(=O)OC(C)(C)C)cc1 ; Cc1ncsc1-c1ccc([C@H](C)N)cc1]

To a solution of tert-butyl N-[(1S)-1-[4-(4-methylthiazol-5-yl)phenyl]ethyl]carbamate (1.5 g, 4.71 mmol, 1 eq) in dichloromethane (20 mL) was added hydrochloride acid/dioxane (4 M, 20 mL, 17 eq). The mixture was stirred at 25° C. for 12 hours. The reaction mixture was concentrated under reduced pressure to remove dichloromethane. The crude product was triturated with petroleum ether (100 mL). Crude (1S)-1-[4-(4-methylthiazol-5-yl)phenyl]ethanamine hydrochloride (1.1 g) was obtained as a yellow solid.

### Step 8

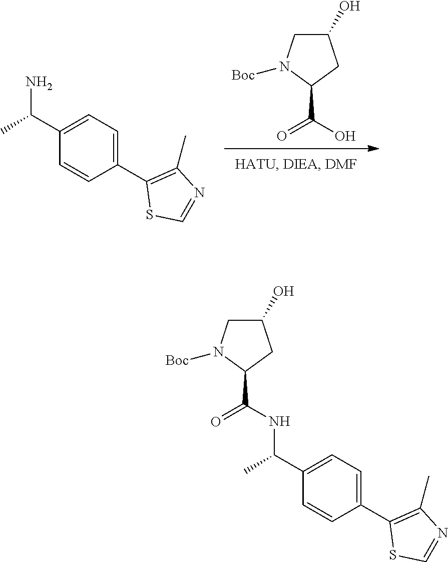 [SMILES: Cc1ncsc1-c1ccc([C@H](C)N)cc1 ; CC(C)(C)OC(=O)N1C[C@H](O)C[C@H]1C(=O)O ; Cc1ncsc1-c1ccc([C@H](C)NC(=O)[C@@H]2C[C@@H](O)CN2C(=O)OC(C)(C)C)cc1]

To a solution of (2S,4R)-1-tert-butoxycarbonyl-4-hydroxy-pyrrolidine-2-carboxylic acid (998 mg, 4.32 mmol, 1.1 eq) and O-(7-azabenzotriazol-1-yl)-N,N,N′,N′-tetramethyluronium hexafluorophosphate (1.79 g, 4.71 mmol, 1.2 eq) in dimethylformamide (10 mL) were added (1S)-1-[4-(4-methylthiazol-5-yl)phenyl]ethanamine hydrochloride (1 g, 3.92 mmol, 1 eq) and diisopropylethyl amine (1.52 g, 11.77 mmol, 2.05 mL, 3 eq). The reaction mixture was stirred at 15° C. for 0.5 hours. The reaction mixture was poured into water (20 mL) and extracted with ethyl acetate (30 mL×3). The combined organic layers were washed with brine (50 mL×3), dried over anhydrous sodium sulfate, filtered, and concentrated under reduced pressure. The residue was purified by silica gel column chromatography (petroleum ether: ethyl acetate from 100:1 to 30:1). tert-Butyl (2S,4R)-4-hydroxy-2-[[(1S)-1-[4-(4-methylthiazol-5-yl)phenyl]ethyl]carbamoyl] pyrrolidine-1-carboxylate (1.2 g, 2.78 mmol, 70% yield) was obtained as a white solid.

### Step 9

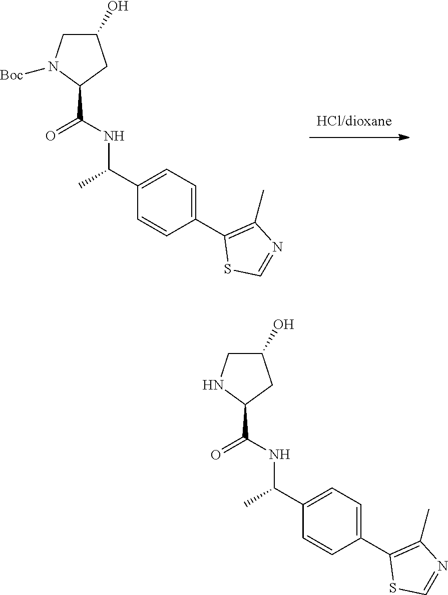 [SMILES: Cc1ncsc1-c1ccc([C@H](C)NC(=O)[C@@H]2C[C@@H](O)CN2C(=O)OC(C)(C)C)cc1 ; Cc1ncsc1-c1ccc([C@H](C)NC(=O)[C@@H]2C[C@@H](O)CN2)cc1]

To a solution of tert-butyl (2S,4R)-4-hydroxy-2-[[(1S)-1-[4-(4-methylthiazol-5-yl) phenyl]ethyl]carbamoyl]pyrrolidine-1-carboxylate (1 g, 2.32 mmol, 1 eq) in dichloromethane (10 mL) was added hydrochloric acid (2.5 M in dioxane, 5 mL, 5.4 eq). The reaction mixture was stirred at 15° C. for 0.5 hours. The reaction mixture was concentrated under reduced pressure. (2S,4R)-4-hydroxy-N-[(1S)-1-[4-(4-methylthiazol-5-yl)phenyl]ethyl]pyrrolidine-2-carboxamide hydrochloride (800 mg, 2.17 mmol, 93% yield) was obtained as a colorless oil.

### Step 10

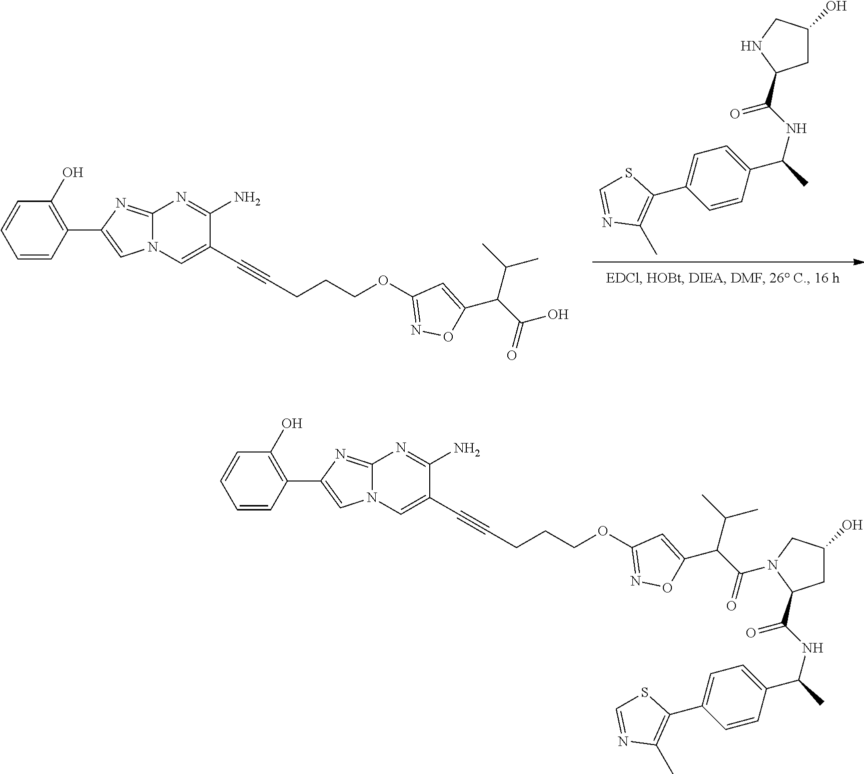 [SMILES: CC(C)C(C(=O)O)c1cc(OCCCC#Cc2cn3cc(-c4ccccc4O)nc3nc2N)no1 ; Cc1ncsc1-c1ccc([C@H](C)NC(=O)[C@@H]2C[C@@H](O)CN2)cc1 ; Cc1ncsc1-c1ccc([C@H](C)NC(=O)[C@@H]2C[C@@H](O)CN2C(=O)C(c2cc(OCCCC#Cc3cn4cc(-c5ccccc5O)nc4nc3N)no2)C(C)C)cc1]

To a mixture of 2-[3-[5-[7-amino-2-(2-hydroxyphenyl)imidazo[1,2-a]pyrimidin-6-yl]pent-4-ynoxy]isoxazole-5-yl]-3-methyl-butanoic acid (60 mg, 0.13 mmol, 1 eq) and (2S,4R)-4-hydroxy-N-[(1S)-1-[4-(4-methylthiazol-5-yl)phenyl]ethyl]pyrrolidine-2-carboxamide (63 mg, 0.19 mmol, 1.5 eq) in N,N-dimethylformamide (2 mL) was added 1-(3-dimethylaminopropyl)-3-ethylcarbodiimide hydrochloride (36 mg, 0.18 mmol, 1.5 eq), hydroxybenzotriazole (25 mg, 0.18 mmol, 1.5 eq) and N,N-diisopropylethylamine (81 mg, 0.63 mmol, 109.90 uL, 5 eq). The mixture was stirred at 26° C. for 16 hours. The mixture was concentrated under reduced pressure at 40° C. The residue was purified by semi-preparative reverse phase HPLC (column: Phenomenex Gemini-NX C18 75*30 mm*3 um; mobile phase: [water (0.1% TFA)-ACN]; B %: 28%-38%, 7 min). Compound (2S,4R)-1-[2-[3-[5-[7-amino-2-(2-hydroxyphenyl)imidazo[1,2-a]pyrimidin-6-yl]pent-4-ynoxy]236soxazole-5-yl]-3-methyl-butanoyl]-4-hydroxy-N-[(1S)-1-[4-(4-methylthiazol-5-yl)phenyl]ethyl]pyrrolidine-2-carboxamide (40 mg, 0.05 mmol) was obtained as a white solid.

### Step 11

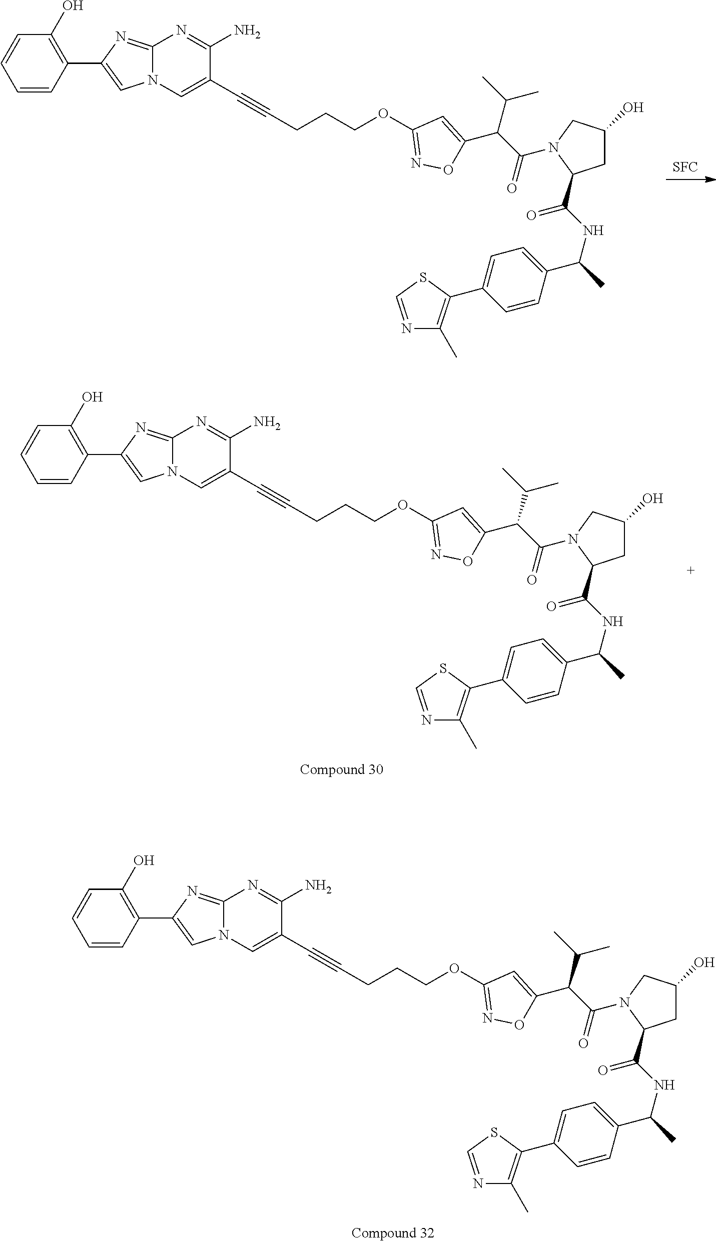 [SMILES: Cc1ncsc1-c1ccc([C@H](C)NC(=O)[C@@H]2C[C@@H](O)CN2C(=O)C(c2cc(OCCCC#Cc3cn4cc(-c5ccccc5O)nc4nc3N)no2)C(C)C)cc1 ; "SFC" ; Cc1ncsc1-c1ccc([C@H](C)NC(=O)[C@@H]2C[C@@H](O)CN2C(=O)[C@H](c2cc(OCCCC#Cc3cn4cc(-c5ccccc5O)nc4nc3N)no2)C(C)C)cc1 ; Cc1ncsc1-c1ccc([C@H](C)NC(=O)[C@@H]2C[C@@H](O)CN2C(=O)[C@@H](c2cc(OCCCC#Cc3cn4cc(-c5ccccc5O)nc4nc3N)no2)C(C)C)cc1]

The compound (2S,4R)-1-[2-[3-[5-[7-amino-2-(2-hydroxyphenyl)imidazo[1,2-a]pyrimidin-6-yl]pent-4-ynoxy]isoxazole-5-yl]-3-methyl-butanoyl]-4-hydroxy-N-[(1S)-1-[4-(4-methylthiazol-5-yl)phenyl]ethyl]pyrrolidine-2-carboxamide (50 mg, 0.06 mmol, 1 eq) was separated by SFC Column: DAICEL CHIRALPAK OD 250×30 mm, I.D., 10 um; Mobile phase: ethanol (0.1% NH3H2O) in CO2 from 55% to 55%; Flow rate: 70 g/min; Wavelength: 220 nm). Peak 1 was additionally purified by semi-preparative reverse phase HPLC (column: Waters Xbridge C18 150*50 mm*10 um; mobile phase: [water (10 mM NH4HCO3)-ACN]; B %: 37%-67%, 10 min). Peak 2 was additionally purified by semi-preparative reverse phase HPLC (column: Waters Xbridge C18 150*50 mm*10 um; mobile phase: [water (10 mM NH₄HCO₃)-ACN]; B %: 37%-67%, 10 min), then additionally purified by semi-preparative reverse phase HPLC (column: Unisil 3-100 C18 Ultra 150*50 mm*3 um; mobile phase: [water (0.225% FA)-ACN]; B %: 23%-53%, 10 min), and then additionally purified by preparative TLC (dichloromethane/methanol=10:1). Compound (2S,4R)-1-[(2R)-2-[3-[5-[7-amino-2-(2-hydroxyphenyl)imidazo[1,2-a]pyrimidin-6-yl]pent-4-ynoxy]isoxazole-5-yl]-3-methyl-butanoyl]-4-hydroxy-N-[(1S)-1-[4-(4-methylthiazol-5-yl)phenyl]ethyl]pyrrolidine-2-carboxamide (6.4 mg, 0.008 mmol, SFC Rt=2.597) and (2S,4R)-1-[(2S)-2-[3-[5-[7-amino-2-(2-hydroxyphenyl)imidazo[1,2-a]pyrimidin-6-yl]pent-4-ynoxy]isoxazole-5-yl]-3-methyl-butanoyl]-4-hydroxy-N-[(1S)-1-[4-(4-methylthiazol-5-yl)phenyl]ethyl]pyrrolidine-2-carboxamide (6.1 mg, 0.007 mmol, SFC Rt=4.308) were obtained as white solids.

### Exemplary Synthesis of Compound 33

### Step 1

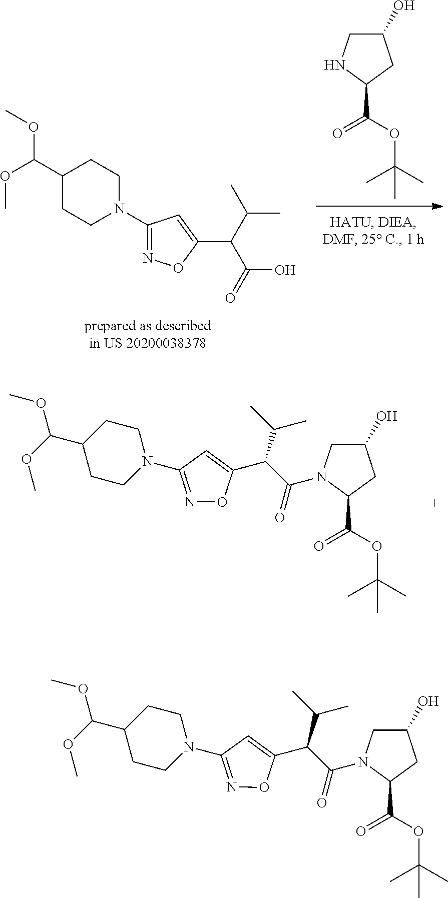

To a solution of 2-[3-[4-(dimethoxymethyl)-1-piperidyl]isoxazole-5-yl]-3-methyl-butanoic acid (3.8 g, 11.64 mmol, 1 eq) [prepared as described in US 20200038378] and tert-butyl (2S,4R)-4-hydroxypyrrolidine-2-carboxylate (3.27 g, 17.46 mmol, 1.5 eq) in N,N-dimethylformamide (10 mL) was added O-(7-azabenzotriazol-1-yl)-N,N,N′,N′-tetramethyluronium hexafluorophosphate (6.64 g, 17.46 mmol, 1.5 eq) and N,N-diisopropylethylamine (4.51 g, 34.93 mmol, 6.1 mL, 3 eq). The mixture was stirred at 25° C. for 1 hour. The reaction mixture was diluted with water (100 mL) and extracted with ethyl acetate (50 mL×3). The combined organic layers were washed with brine 30 mL, dried over anhydrous sodium sulfate, filtered, and concentrated under reduced pressure. The residue was purified by prep-HPLC (column: Phenomenex luna C18 250*80 mm*10 um; mobile phase: [water (0.225% FA)-ACN]; B %: 30%-55%, 20 min). Compound tert-butyl (2S,4R)-1-[(2S)-2-[3-[4-(dimethoxymethyl)-1-piperidyl]237soxazole-5-yl]-3-methyl-butanoyl]-4-hydroxy-pyrrolidine-2-carboxylate (910 mg, 1.84 mmol, 16% yield) was obtained as a yellow oil. Compound tert-butyl (2S,4R)-1-[(2R)-2-[3-[4-(dimethoxymethyl)-1-piperidyl]237soxazole-5-yl]-3-methyl-butanoyl]-4-hydroxy-pyrrolidine-2-carboxylate (820 mg, 1.65 mmol, 14% yield) was obtained as a yellow solid.

### Step 2

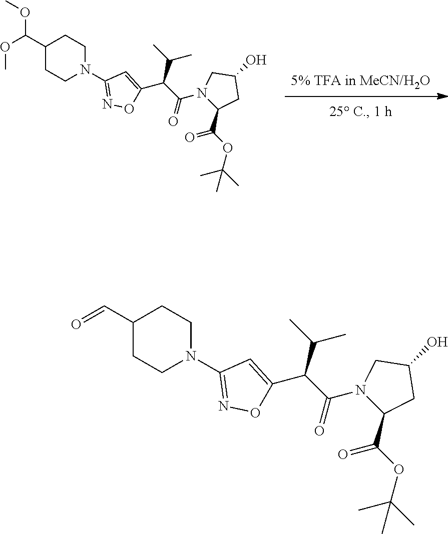 [SMILES: COC(OC)C1CCN(c2cc([C@H](C(=O)N3C[C@H](O)C[C@H]3C(=O)OC(C)(C)C)C(C)C)on2)CC1 ; CC(C)[C@@H](C(=O)N1C[C@H](O)C[C@H]1C(=O)OC(C)(C)C)c1cc(N2CCC(C=O)CC2)no1]

To a solution of tert-butyl (2S,4R)-1-[(2R)-2-[3-[4-(dimethoxymethyl)-1-piperidyl]isoxazole-5-yl]-3-methyl-butanoyl]-4-hydroxy-pyrrolidine-2-carboxylate (100 mg, 0.20 mmol, 1 eq) in acetonitrile (2.5 mL) and water (2.5 mL) was added trifluoroacetic acid (385 mg, 3.38 mmol, 16.73 eq). The mixture was stirred at 25° C. for 1 hour. The reaction mixture was diluted with saturated aqueous sodium bicarbonate solution (10 mL) and extracted with ethyl acetate (20 mL×3). The combined organic layers were washed with brine (30 mL), dried over anhydrous sodium sulfate, filtered, and concentrated under reduced pressure to give a residue. Crude tert-butyl (2S,4R)-1-[(2R)-2-[3-(4-formyl-1-piperidyl)isoxazole-5-yl]-3-methyl-butanoyl]-4-hydroxy-pyrrolidine-2-carboxylate (90 mg, 0.20 mmol) was obtained as a white solid.

### Step 3

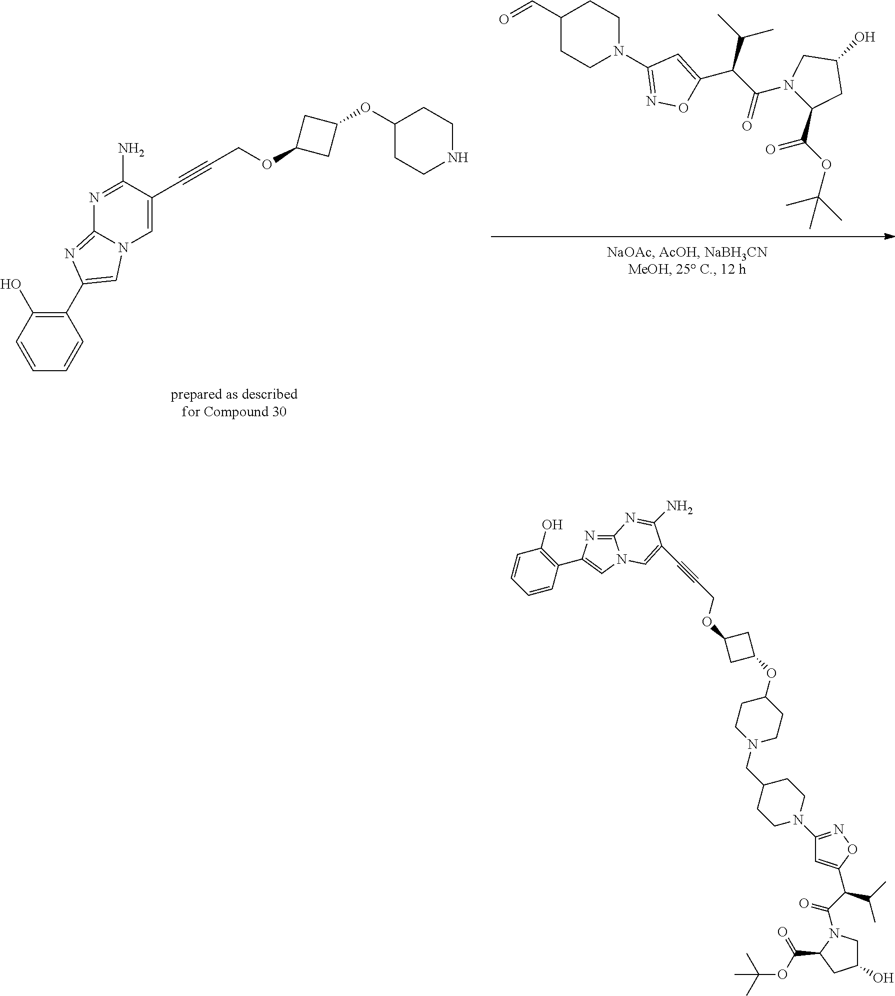 [SMILES: Nc1nc2nc(-c3ccccc3O)cn2cc1C#CCO[C@H]1C[C@H](OC2CCNCC2)C1 ; CC(C)[C@@H](C(=O)N1C[C@H](O)C[C@H]1C(=O)OC(C)(C)C)c1cc(N2CCC(C=O)CC2)no1 ; CC(C)[C@@H](C(=O)N1C[C@H](O)C[C@H]1C(=O)OC(C)(C)C)c1cc(N2CCC(CN3CCC(O[C@H]4C[C@H](OCC#Cc5cn6cc(-c7ccccc7O)nc6nc5N)C4)CC3)CC2)no1]

To a solution of 2-[7-amino-6-[3-[3-(4-piperidyloxy)cyclobutoxy]prop-1-ynyl]imidazo[1,2-a]pyrimidin-2-yl]phenol (72 mg, 0.17 mmol, 1 eq), tert-butyl (2S,4R)-1-[(2R)-2-[3-(4-formyl-1-piperidyl)isoxazole-5-yl]-3-methyl-butanoyl]-4-hydroxy-pyrrolidine-2-carboxylate (75 mg, 0.17 mmol, 1 eq) in methanol (5 mL) were added sodium acetate (41 mg, 0.5 mol, 3 eq), acetic acid (40 mg, 0.66 mmol, 4 eq), and sodium cyanoborohydride (21 mg, 0.33 mol, 2 eq). The mixture was stirred at 25° C. for 12 hours. The reaction mixture was concentrated under reduced pressure. The residue was purified by prep-TLC (dichloromethane:methanol=9:1). Compound tert-butyl (2S,4R)-1-[(2R)-2-[3-[4-[[4-[3-[3-[7-amino-2-(2-hydroxyphenyl)imidazo[1,2-a]pyrimidin-6-yl]prop-2-ynoxy]cyclobutoxy]-1-piperidyl]methyl]-1-piperidyl]isoxazole-5-yl]-3-methyl-butanoyl]-4-hydroxy-pyrrolidine-2-carboxylate (100 mg, 0.10 mmol) was obtained as a yellow solid.

### Step 4

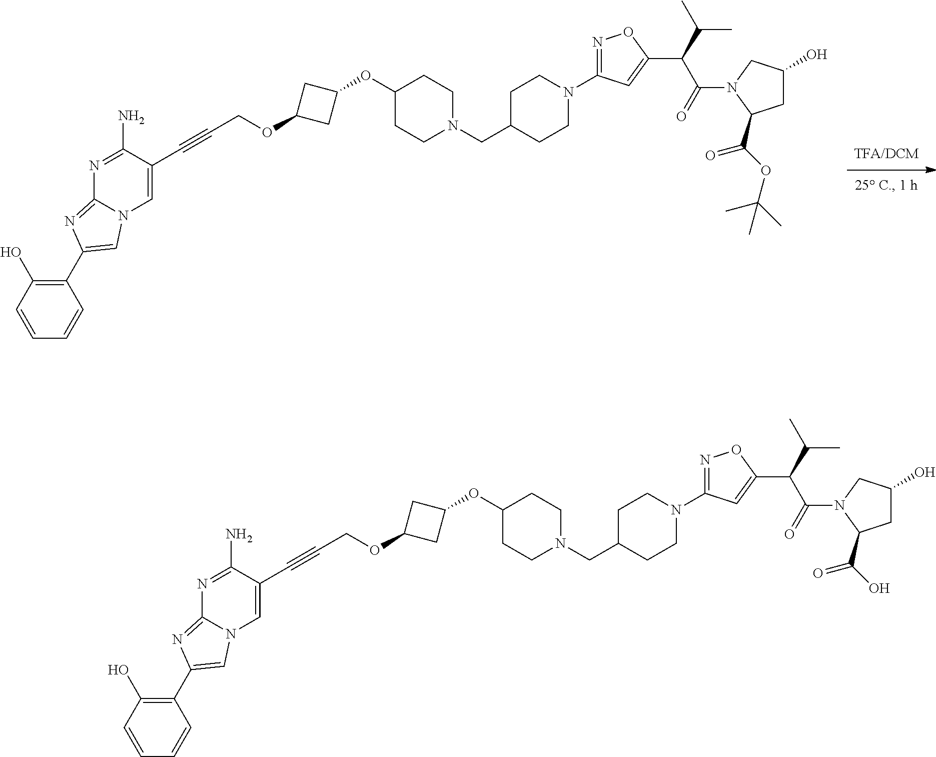 [SMILES: CC(C)[C@@H](C(=O)N1C[C@H](O)C[C@H]1C(=O)OC(C)(C)C)c1cc(N2CCC(CN3CCC(O[C@H]4C[C@H](OCC#Cc5cn6cc(-c7ccccc7O)nc6nc5N)C4)CC3)CC2)no1 ; CC(C)[C@@H](C(=O)N1C[C@H](O)C[C@H]1C(=O)O)c1cc(N2CCC(CN3CCC(O[C@H]4C[C@H](OCC#Cc5cn6cc(-c7ccccc7O)nc6nc5N)C4)CC3)CC2)no1]

To a solution of tert-butyl (2S,4R)-1-[(2R)-2-[3-[4-[[4-[3-[3-[7-amino-2-(2-hydroxyphenyl) imidazo[1,2-a]pyrimidin-6-yl]prop-2-ynoxy]cyclobutoxy]-1-piperidyl]methyl]-1-piperidyl]isoxazole-5-yl]-3-methyl-butanoyl]-4-hydroxy-pyrrolidine-2-carboxylate (100 mg, 0.12 mmol, 1 eq) in dichloromethane (5 mL) was added trifluoroacetic acid (1.54 g, 13.51 mmol, 1 mL, 117.10 eq). The mixture was stirred at 25° C. for 1 h. The reaction mixture was concentrated under reduced pressure. Compound (2S,4R)-1-[(2R)-2-[3-[4-[[4-[3-[3-[7-amino-2-(2-hydroxyphenyl)imidazo[1,2-a]pyrimidin-6-yl]prop-2-ynoxy]cyclobutoxy]-1-piperidyl]methyl]-1-piperidyl]isoxazole-5-yl]-3-methyl-butanoyl]-4-hydroxy-pyrrolidine-2-carboxylic acid (93 mg, 0.11 mmol) was obtained as a yellow solid.

### Step 5

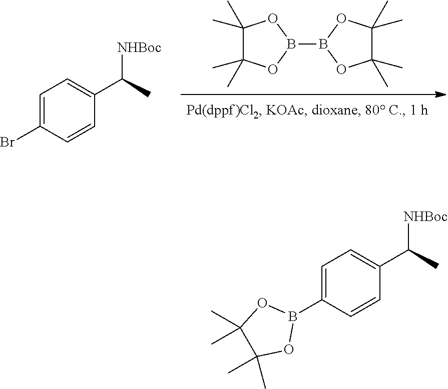

A flask was charged with tert-butyl N-[(1S)-1-(4-bromophenyl)ethyl]carbamate (1.4 g, 4.66 mmol, 1 eq), 4,4,5,5-tetramethyl-2-(4,4,5,5-tetramethyl-1,3,2-dioxaborolan-2-yl)-1,3,2-dioxaborolane (1.42 g, 5.60 mmol, 1.2 eq), (1,1′-bis(diphenylphosphino)ferrocene)palladium(II) dichloride (170 mg, 0.23 mmol, 0.05 eq), potassium acetate (915 mg, 9.33 mmol, 2 eq) and dioxane (30 mL). The mixture was purged with nitrogen for 10 minutes, then heated to 80° C. for 1 hour. The reaction mixture was cooled to 20° C. and filtered through a pad of celite. The filtrate was concentrated in vacuum. The crude product was purified by silica gel column chromatography (petroleum ether: ethyl acetate=10:1). Tert-Butyl N-[(1S)-1-[4-(4,4,5,5-tetramethyl-1,3,2-dioxaborolan-2-yl)phenyl]ethyl]carbamate (1.9 g) was obtained as a colorless oil.

### Step 6

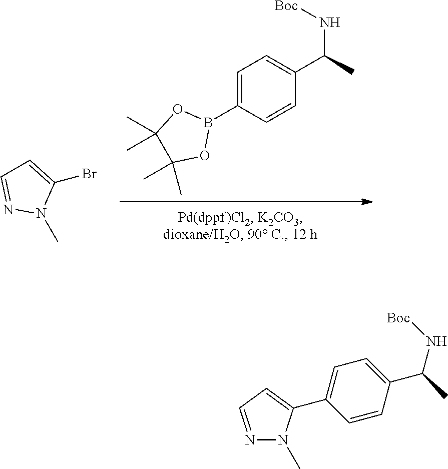

To a mixture of 5-bromo-1-methylpyrazole (800 mg, 4.97 mmol, 1 eq), potassium carbonate (1.37 g, 9.94 mmol, 2 eq) and tert-butyl N-(5-bromothiazol-4-yl)carbamate (2.07 g, 5.96 mmol, 1.2 eq) in water (5 mL) and dioxane (30 mL) was added [1,1′-bis(diphenylphosphino) ferrocene]dichloropalladium(ii) (290 mg, 0.40 mmol, 0.08 eq) at 20° C. under nitrogen. The mixture was stirred at 90° C. for 12 hours. The mixture was cooled to 20° C., poured into ice-water (w/w=1/1, 50 mL) and stirred for 10 minutes. The aqueous phase was extracted with ethyl acetate (40 mL×3). The combined organic phase was washed with brine (20 mL×2), dried with anhydrous sodium sulfate, filtered, and concentrated in vacuum. The residue was purified by silica gel chromatography (petroleum ether/ethyl acetate=30/1 to 5/1) to afford tert-butyl N-[(1S)-1-[4-(2-methylpyrazol-3-yl)phenyl]ethyl]carbamate (1.6 g, crude) as a yellow solid.

### Step 7

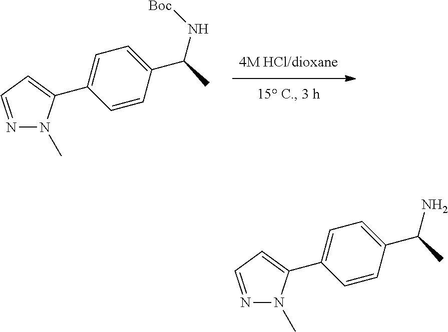 [SMILES: C[C@H](NC(=O)OC(C)(C)C)c1ccc(-c2ccnn2C)cc1 ; C[C@H](N)c1ccc(-c2ccnn2C)cc1]

A solution of tert-butyl N-[(1S)-1-[4-(2-methylpyrazol-3-yl)phenyl]ethyl]carbamate (1.6 g, 5.31 mmol, 1 eq) in hydrochloric acid/dioxane (4 M, 20 mL) was stirred at 15° C. for 3 hours. The mixture was concentrated under reduced pressure at 40° C. The residue was purified by semi-preparative reverse phase HPLC (column: Phenomenex Synergi Max-RP 250*50 mm*10 um; mobile phase: [water (0.1% TFA)-ACN]; B %: 5%-35%, 10 min). Compound (1S)-1-[4-(2-methylpyrazol-3-yl)phenyl]ethanamine trifluoroacetate (1.4 g) was obtained as a yellow oil.

### Step 8

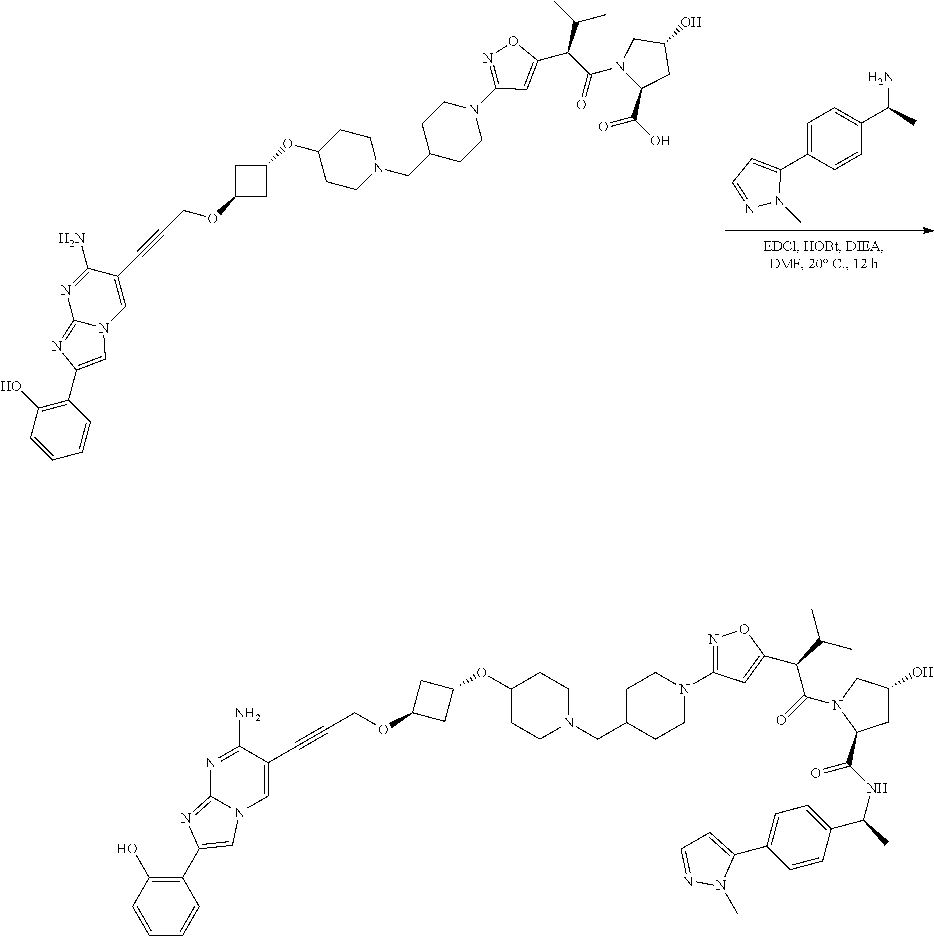 [SMILES: CC(C)[C@@H](C(=O)N1C[C@H](O)C[C@H]1C(=O)O)c1cc(N2CCC(CN3CCC(O[C@H]4C[C@H](OCC#Cc5cn6cc(-c7ccccc7O)nc6nc5N)C4)CC3)CC2)no1 ; C[C@H](N)c1ccc(-c2ccnn2C)cc1 ; CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](OCC#Cc5cn6cc(-c7ccccc7O)nc6nc5N)C4)CC3)CC2)no1]

To a solution of (2S,4R)-1-[(2R)-2-[3-[4-[[4-[3-[3-[7-amino-2-(2-hydroxyphenyl)imidazo [1,2-a]pyrimidin-6-yl]prop-2-ynoxy]cyclobutoxy]-1-piperidyl]methyl]-1-piperidyl]isoxazole-5-yl]-3-methyl-butanoyl]-4-hydroxy-pyrrolidine-2-carboxylic acid (93 mg, 0.11 mmol, 1 eq) and (1S)-1-[4-(2-methylpyrazol-3-yl)phenyl]ethanamine (46 mg, 0.23 mmol, 2 eq) in N,N-dimethylformamide (3 mL) was added N,N-diisopropylethylamine (74 mg, 0.57 mmol, 5 eq), 1-hydroxybenzotriazole (23 mg, 0.17 mmol, 1.5 eq) and 1-(3-dimethylaminopropyl)-3-ethylcarbodiimide hydrochloride (43.97 mg, 0.23 mmol, 2 eq). The mixture was stirred at 25° C. for 12 hours. The reaction mixture was filtered to give a residue. The residue was purified by prep-HPLC (column: Unisil 3-100 C18 Ultra 150*50 mm*3 um; mobile phase: [water (0.225% FA)-ACN]; B %: 18%-38%, 10 min), followed by an additional purification by prep-HPLC (column: Waters Xbridge C18 150*50 mm*10 um; mobile phase: [water (10 mM NH4HCO3)-ACN]; B %: 36%-66%, 10 min) again. Compound (2S,4R)-1-[(2R)-2-[3-[4-[[4-[3-[3-[7-amino-2-(2-hydroxyphenyl)imidazo[1,2-a]pyrimidin-6-yl]prop-2-ynoxy]cyclobutoxy]-1-piperidyl]methyl]-1-piperidyl]242soxazole-5-yl]-3-methyl-butanoyl]-4-hydroxy-N-[(1S)-1-[4-(2-methylpyrazol-3-yl)phenyl]ethyl]pyrrolidine-2-carboxamide (17.0 mg, 0.02 mmol) was obtained as a yellow solid.

### Exemplary Synthesis of Compound 34

Prepared according to the scheme below using procedures described for Exemplary Compounds 29 and 33 above.

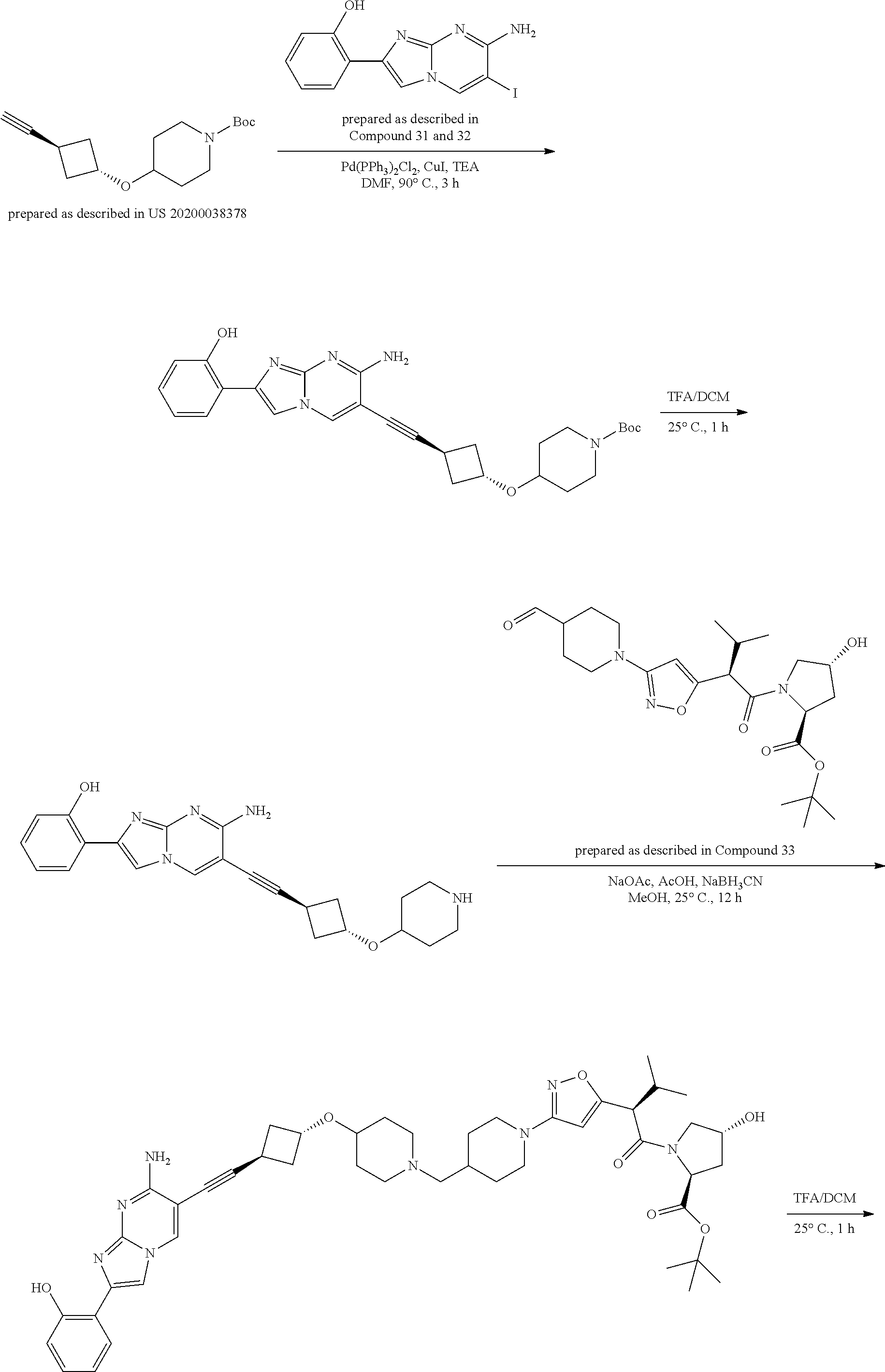 [SMILES: Nc1nc2nc(-c3ccccc3O)cn2cc1I ; C#C[C@H]1C[C@H](OC2CCN(C(=O)OC(C)(C)C)CC2)C1 ; CC(C)(C)OC(=O)N1CCC(O[C@H]2C[C@H](C#Cc3cn4cc(-c5ccccc5O)nc4nc3N)C2)CC1 ; Nc1nc2nc(-c3ccccc3O)cn2cc1C#C[C@H]1C[C@H](OC2CCNCC2)C1 ; CC(C)[C@@H](C(=O)N1C[C@H](O)C[C@H]1C(=O)OC(C)(C)C)c1cc(N2CCC(C=O)CC2)no1 ; CC(C)[C@@H](C(=O)N1C[C@H](O)C[C@H]1C(=O)OC(C)(C)C)c1cc(N2CCC(CN3CCC(O[C@H]4C[C@H](C#Cc5cn6cc(-c7ccccc7O)nc6nc5N)C4)CC3)CC2)no1]

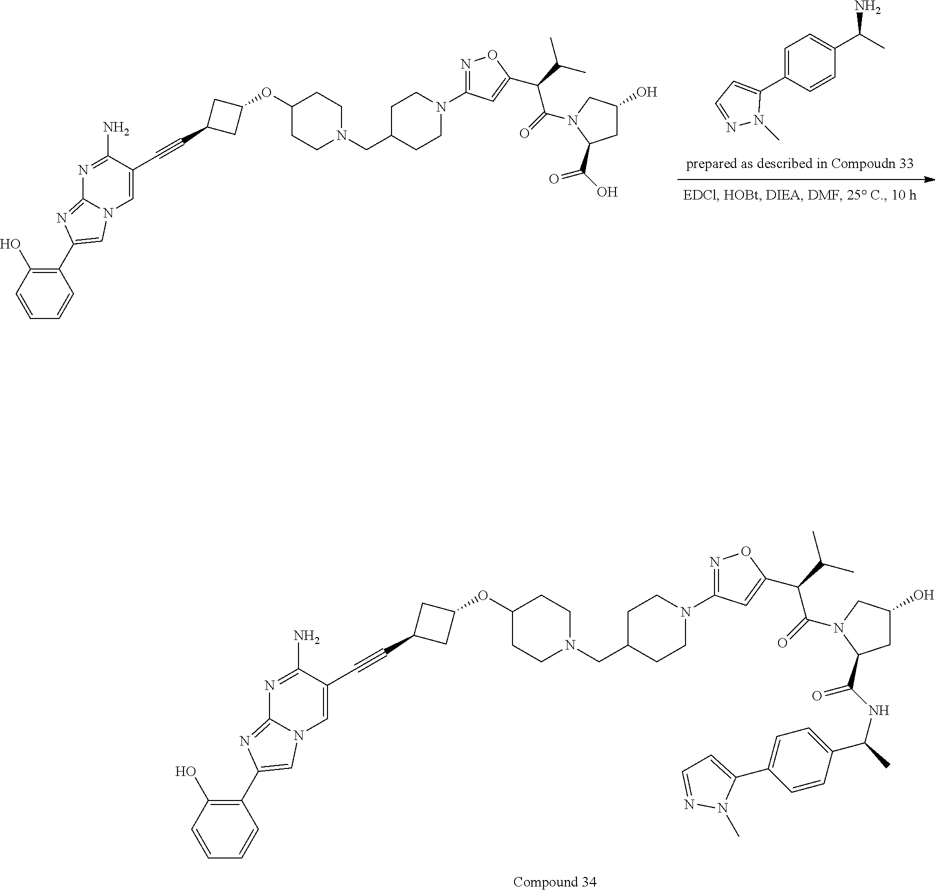 [SMILES: CC(C)[C@@H](C(=O)N1C[C@H](O)C[C@H]1C(=O)O)c1cc(N2CCC(CN3CCC(O[C@H]4C[C@H](C#Cc5cn6cc(-c7ccccc7O)nc6nc5N)C4)CC3)CC2)no1 ; C[C@H](N)c1ccc(-c2ccnn2C)cc1 ; CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](C#Cc5cn6cc(-c7ccccc7O)nc6nc5N)C4)CC3)CC2)no1]

### Exemplary Synthesis of Compounds 34 and 35

### Step 1

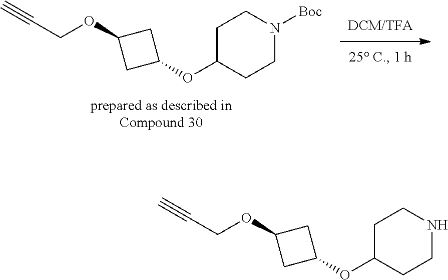 [SMILES: C#CCO[C@H]1C[C@H](OC2CCN(C(=O)OC(C)(C)C)CC2)C1 ; C#CCO[C@H]1C[C@H](OC2CCNCC2)C1]

To a solution of tert-butyl 4-(3-prop-2-ynoxycyclobutoxy)piperidine-1-carboxylate (1 g, 3.23 mmol, 1 eq) in dichloromethane (10 mL) was added trifluoroacetic acid (3.08 g, 27.01 mmol, 2 mL, 8.36 eq). The mixture was stirred at 25° C. for 1 hour. The reaction mixture was concentrated under reduced pressure. Crude 4-(3-prop-2-ynoxycyclobutoxy) piperidine trifluoroacetate (1.01 g, 3.12 mmol) was obtained as a yellow oil.

### Step 2

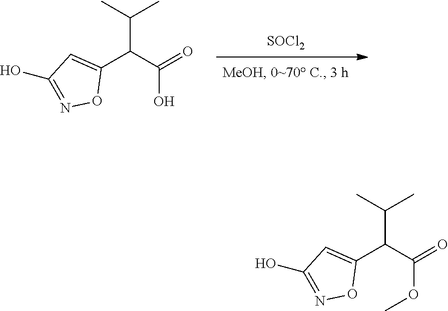

To a solution of 2-(3-hydroxyisoxazol-5-yl)-3-methyl-butanoic acid (1 g, 5.40 mmol, 1 eq) in methanol (10 mL) was added thionyl chloride (2.57 g, 21 mmol, 1.57 mL, 4 eq) at 0° C. The reaction mixture was stirred at 70° C. for 3 hours. The reaction mixture was concentrated under reduced pressure. The residue was diluted with water (50 ml) and extracted with ethyl acetate (30 mL×3). The combined organic layers were washed with brine (80 mL×2), dried over anhydrous sodium sulfate, filtered, and concentrated under reduced pressure. Methyl 2-(3-hydroxyisoxazol-5-yl)-3-methyl-butanoate (1 g, 5.02 mmol, 92% yield) was obtained as a yellow oil.

### Step 3

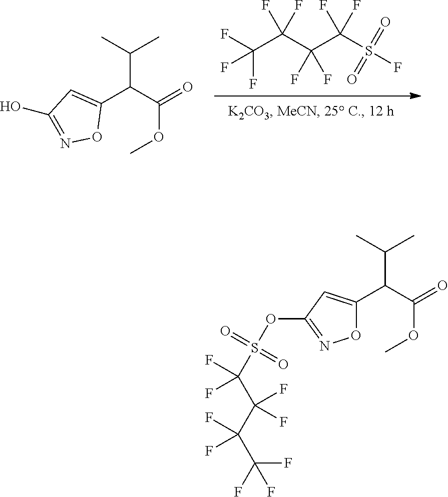

To a solution of methyl 2-(3-hydroxyisoxazol-5-yl)-3-methyl-butanoate (800 mg, 4.02 mmol, 1 eq) in acetonitrile (5 mL) was added potassium carbonate (1.11 g, 8.03 mmol, 2 eq) and perfluorobutyl sulfonyl fluoride (1.46 g, 4.82 mmol, 1.2 eq). The reaction mixture was stirred at 25° C. for 12 hours. The reaction mixture was diluted with water (50 mL) and extracted with ethyl acetate (30 mL×3). The combined organic layers were washed with brine (80 mL×3), dried over anhydrous sodium sulfate, filtered, and concentrated under reduced pressure. The residue was purified by silica gel column chromatography (petroleum ether/ethyl acetate=100/1 to 20/1). Methyl 3-methyl-2-[3-(1,1,2,2,3,3,4,4,4-nonafluorobutylsulfonyloxy)243soxazole-5-yl]butanoate (530 mg, 1.10 mmol) was obtained as a colorless oil.

### Step 4

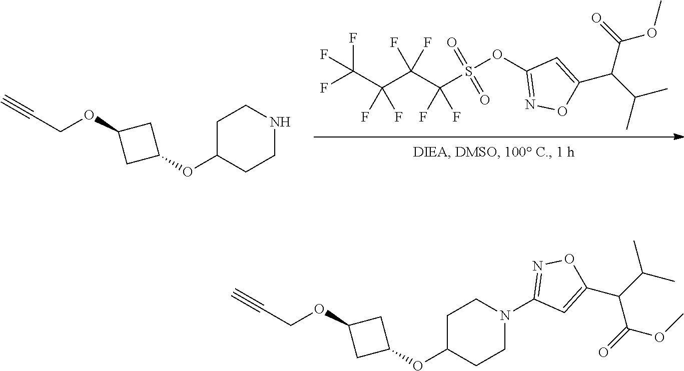 [SMILES: C#CCO[C@H]1C[C@H](OC2CCNCC2)C1 ; COC(=O)C(c1cc(OS(=O)(=O)C(F)(F)C(F)(F)C(F)(F)C(F)(F)F)no1)C(C)C ; C#CCO[C@H]1C[C@H](OC2CCN(c3cc(C(C(=O)OC)C(C)C)on3)CC2)C1]

To a solution of 4-(3-prop-2-ynoxycyclobutoxy)piperidine (1 g, 3.09 mmol, 1 eq, trifluoroacetate) in methyl sulfoxide (10 mL) was added N,N-diisopropylethylamine (799 mg, 6.19 mmol, 1.1 mL, 2 eq), then methyl 3-methyl-2-[3-(1,1,2,2,3,3,4,4,4-nonafluorobutylsulfonyloxy)isoxazole-5-yl]butanoate (2.23 g, 4.64 mmol, 1.5 eq) was added dropwise at 100° C. The mixture was stirred at 100° C. for 1 hour. The reaction mixture was diluted with water (100 mL) and extracted with ethyl acetate (50 mL×3). The combined organic layers were washed with brine 30 mL, dried over anhydrous sodium sulfate, filtered, and concentrated under reduced pressure. The residue was purified by silica gel chromatography (petroleum ether/ethyl acetate=20/1 to 1/1). Compound methyl 3-methyl-2-[3-[4-(3-prop-2-ynoxycyclobutoxy)-1-piperidyl]isoxazole-5-yl]butanoate (255 mg, 0.54 mmol) was obtained as a yellow oil.

### Step 5

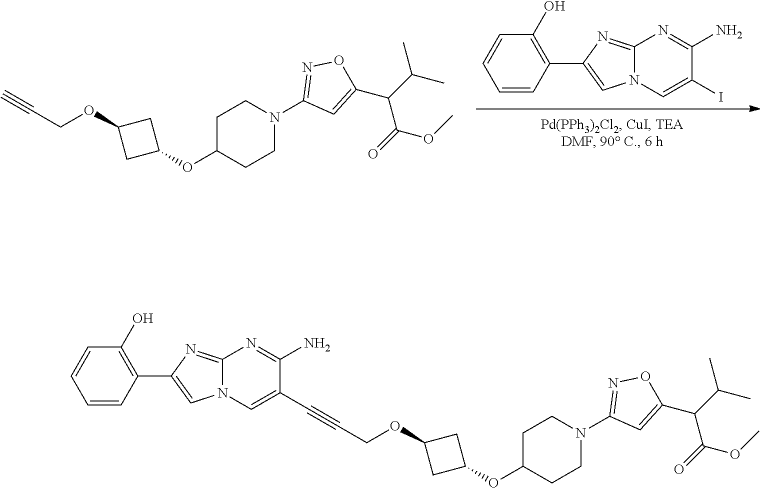

To a solution of methyl 3-methyl-2-[3-[4-(3-prop-2-ynoxycyclobutoxy)-1-piperidyl]isoxazole-5-yl]butanoate (250 mg, 0.64 mmol, 1 eq), 2-(7-amino-6-iodo-imidazo[1,2-a]pyrimidin-2-yl)phenol (225 mg, 0.64 mol, 1 eq) in N,N-dimethylformamide (5 mL) was added bis (triphenylphosphine)palladium(II) chloride (45 mg, 0.064 mol, 0.1 eq), triethylamine (129 mg, 1.28 mmol, 2 eq) and copper iodide (24 mg, 0.13 mmol, 0.2 eq). The mixture was stirred at 90° C. for 6 hours under nitrogen. The reaction mixture was diluted with water 20 mL and extracted with ethyl acetate (50 mL×3). The combined organic layers were washed with brine 30 mL, dried over anhydrous sodium sulfate, filtered, and concentrated under reduced pressure. The residue was purified by prep-HPLC (column: Phenomenex luna C18 150*40 mm*15 um; mobile phase: [water (0.1% TFA)-ACN]; B %: 28%-58%, 11 min). Compound methyl 2-[3-[4-[3-[3-[7-amino-2-(2-hydroxyphenyl)imidazo[1,2-a]pyrimidin-6-yl]prop-2-ynoxy]cyclobutoxy]-1-piperidyl]isoxazole-5-yl]-3-methyl-butanoate (161 mg, 0.25 mmol) was obtained as a white solid.

### Step 6

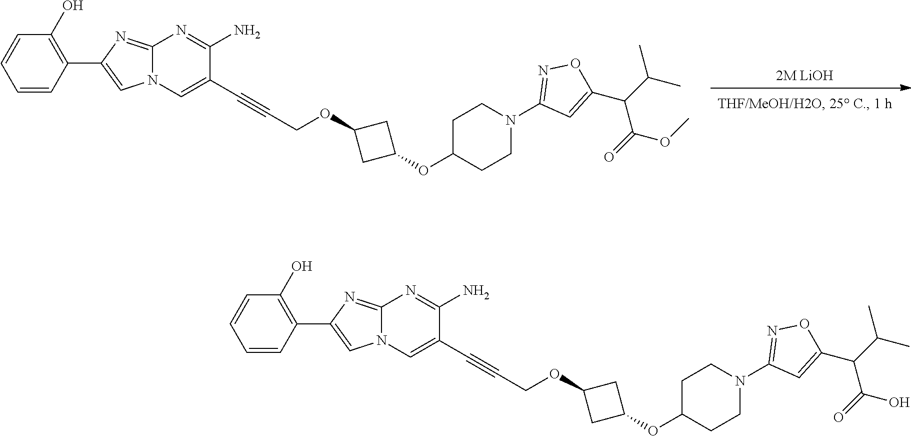 [SMILES: COC(=O)C(c1cc(N2CCC(O[C@H]3C[C@H](OCC#Cc4cn5cc(-c6ccccc6O)nc5nc4N)C3)CC2)no1)C(C)C ; "2M LiOH" ; CC(C)C(C(=O)O)c1cc(N2CCC(O[C@H]3C[C@H](OCC#Cc4cn5cc(-c6ccccc6O)nc5nc4N)C3)CC2)no1]

To a solution of methyl 2-[3-[4-[3-[3-[7-amino-2-(2-hydroxyphenyl)imidazo[1,2-a] pyrimidin-6-yl]prop-2-ynoxy]cyclobutoxy]-1-piperidyl]245soxazole-5-yl]-3-methyl-butanoate (160 mg, 0.26 mmol, 1 eq) in tetrahydrofuran (2 mL) and methanol (2 mL) was added lithium hydroxide (2 M, 2 mL, 15 eq). The mixture was stirred at 25° C. for 1 hours. The reaction mixture was concentrated under reduced pressure to remove tetrahydrofuran, then filtered to give a residue. The residue was purified by prep-HPLC (column: Phenomenex Gemini-NX C18 75*30 mm*3 um; mobile phase: [water (0.1% TFA)-ACN]; B %: 28%-38%, 7 min). Compound 2-[3-[4-[3-[3-[7-amino-2-(2-hydroxyphenyl)imidazo[1,2-a]pyrimidin-6-yl]prop-2-ynoxy]cyclobutoxy]-1-piperidyl]isoxazole-5-yl]-3-methyl-butanoic acid (70 mg, 0.12 mmol) was obtained as a yellow solid.

### Step 7

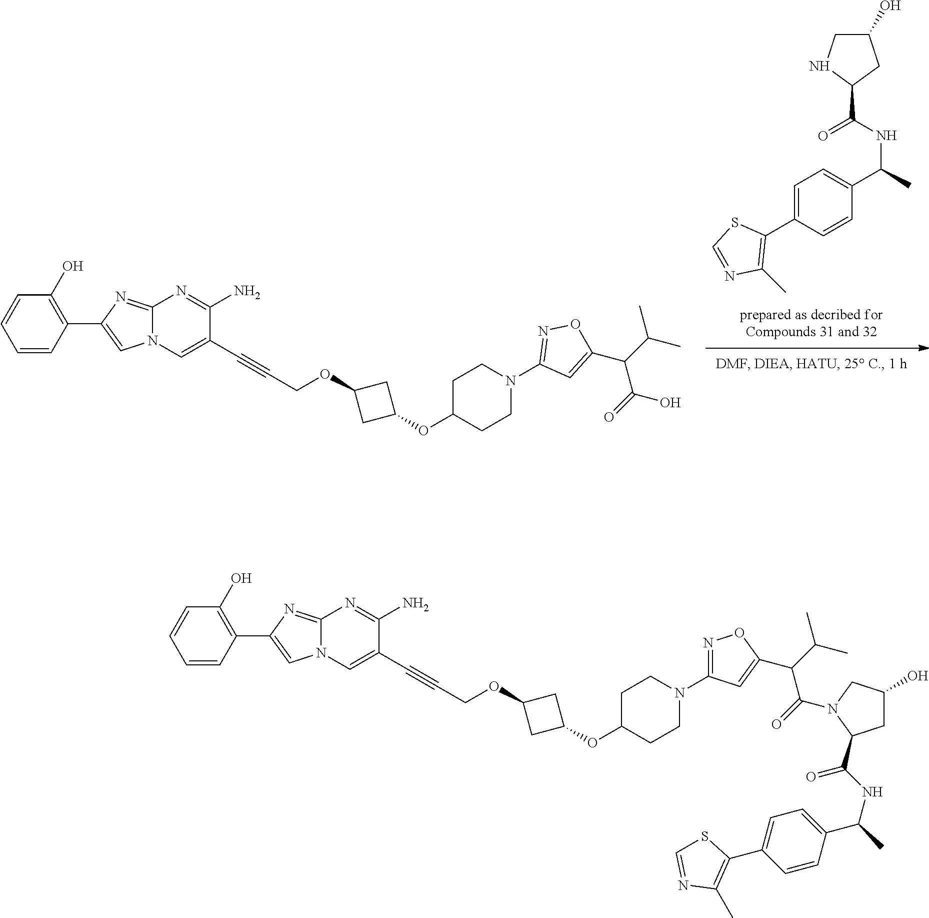 [SMILES: CC(C)C(C(=O)O)c1cc(N2CCC(O[C@H]3C[C@H](OCC#Cc4cn5cc(-c6ccccc6O)nc5nc4N)C3)CC2)no1 ; Cc1ncsc1-c1ccc([C@H](C)NC(=O)[C@@H]2C[C@@H](O)CN2)cc1 ; Cc1ncsc1-c1ccc([C@H](C)NC(=O)[C@@H]2C[C@@H](O)CN2C(=O)C(c2cc(N3CCC(O[C@H]4C[C@H](OCC#Cc5cn6cc(-c7ccccc7O)nc6nc5N)C4)CC3)no2)C(C)C)cc1]

To a solution of 2-[3-[4-[3-[3-[7-amino-2-(2-hydroxyphenyl)imidazo[1,2-a]pyrimidin-6-yl] prop-2-ynoxy]cyclobutoxy]-1-piperidyl]isoxazole-5-yl]-3-methyl-butanoic acid (70 mg, 0.12 mmol, 1 eq) and (2S,4R)-4-hydroxy-N-[(1S)-1-[4-(4-methylthiazol-5-yl)phenyl]ethyl] pyrrolidine-2-carboxamide (43 mg, 0.12 mmol, 1 eq, hydrochloride) in N,N-dimethylformamide (3 mL) was added N,N-diisopropylethylamine (45 mg, 0.35 mmol, 3 eq) and O-(7-azabenzotriazol-1-yl)-N,N,N′,N′-tetramethyluronium hexafluorophosphate (66 mg, 0.17 mmol, 1.5 eq) at 0° C. The mixture was stirred at 25° C. for 1 hour. The reaction mixture was filtered to give a residue. The residue was purified by prep-HPLC (column: Unisil 3-100 C18 Ultra 150*50 mm*3 um; mobile phase: [water (0.225% FA)-ACN]; B %: 30%-50%, 10 min). Compound (2S,4R)-1-[2-[3-[4-[3-[3-[7-amino-2-(2-hydroxyphenyl)imidazo[1,2-a]pyrimidin-6-yl]prop-2-ynoxy]cyclobutoxy]-1-piperidyl]isoxazole-5-yl]-3-methyl-butanoyl]-4-hydroxy-N-[(1S)-1-[4-(4-methylthiazol-5-yl)phenyl]ethyl]pyrrolidine-2-carboxamide formate (80 mg, 0.08 mmol) was obtained as a yellow solid.

### Step 8

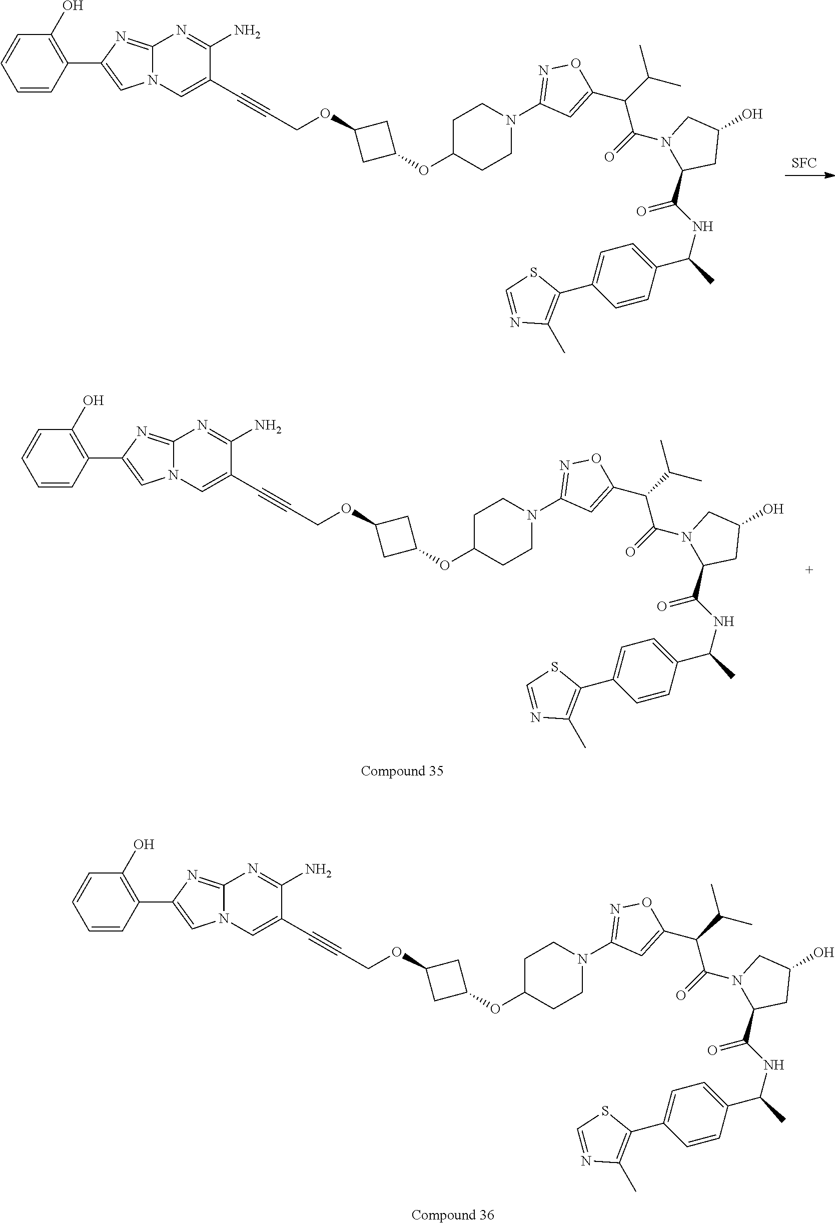 [SMILES: Cc1ncsc1-c1ccc([C@H](C)NC(=O)[C@@H]2C[C@@H](O)CN2C(=O)C(c2cc(N3CCC(O[C@H]4C[C@H](OCC#Cc5cn6cc(-c7ccccc7O)nc6nc5N)C4)CC3)no2)C(C)C)cc1 ; "SFC" ; Cc1ncsc1-c1ccc([C@H](C)NC(=O)[C@@H]2C[C@@H](O)CN2C(=O)[C@H](c2cc(N3CCC(O[C@H]4C[C@H](OCC#Cc5cn6cc(-c7ccccc7O)nc6nc5N)C4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(O[C@H]4C[C@H](OCC#Cc5cn6cc(-c7ccccc7O)nc6nc5N)C4)CC3)no2)C(C)C)cc1]

(2S,4R)-1-[2-[3-[4-[3-[3-[7-amino-2-(2-hydroxyphenyl)imidazo[1,2-a]pyrimidin-6-yl]prop-2-ynoxy]cyclobutoxy]-1-piperidyl]isoxazole-5-yl]-3-methyl-butanoyl]-4-hydroxy-N-[(1S)-1-[4-(4-methylthiazol-5-yl)phenyl]ethyl]pyrrolidine-2-carboxamide (70 mg, 0.07 mmol, 1 eq) was separated by prep-SFC (column: DAICEL CHIRALPAK AD 250 mm*30 mm, I.D., 10 um; mobile phase: isopropanol (0.1% NH3H2O) in CO2 from 60% to 60%; Flow rate: 70 g/min; Wavelength: 220 nm) to give product 1 and product 2. Product 1 was then purified by prep-HPLC (column: Unisil 3-100 C18 Ultra 150*50 mm*3 um; mobile phase: [water (0.225% FA)-ACN]; B %: 30%-50%, 10 min), and product 2 was purified by pre-HPLC (column: Unisil 3-100 C18 Ultra 150*50 mm*3 um; mobile phase: [water (0.225% FA)-ACN]; B %: 30%-50%, 10 min). Product 1: (2S,4R)-1-[(2S)-2-[3-[4-[3-[3-[7-amino-2-(2-hydroxyphenyl)imidazo[1,2-a]pyrimidin-6-yl]prop-2-ynoxy]cyclobutoxy]-1-piperidyl]isoxazole-5-yl]-3-methyl-butanoyl]-4-hydroxy-N-[(1S)-1-[4-(4-methylthiazol-5-yl)phenyl]ethyl]pyrrolidine-2-carboxamide formate (7.8 mg, 0.008 mmol, 11% yield) was obtained as a white solid. Product 2: (2S,4R)-1-[(2R)-2-[3-[4-[3-[3-[7-amino-2-(2-hydroxyphenyl)imidazo[1,2-a]pyrimidin-6-yl]prop-2-ynoxy]cyclobutoxy]-1-piperidyl]isoxazole-5-yl]-3-methyl-butanoyl]-4-hydroxy-N-[(1S)-1-[4-(4-methylthiazol-5-yl)phenyl]ethyl]pyrrolidine-2-carboxamide formate (25.5 mg, 0.026 mmol) was obtained as a white solid.

### Exemplary Synthesis of Compounds 37 and 38

### Step 1

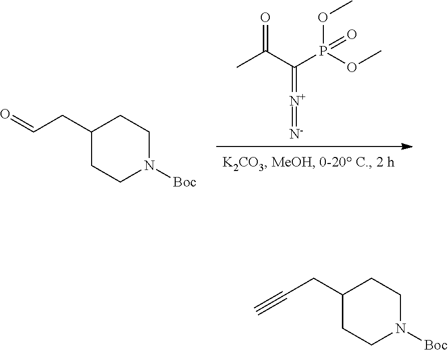 [SMILES: CC(C)(C)OC(=O)N1CCC(CC=O)CC1 ; COP(=O)(OC)C(=[N+]=[N-])C(C)=O ; C#CCC1CCN(C(=O)OC(C)(C)C)CC1]

To a mixture of tert-butyl 4-(2-oxoethyl)piperidine-1-carboxylate (4 g, 17.60 mmol, 1 eq) and 1-diazo-1-dimethoxyphosphoryl-propan-2-one (4.06 g, 21.12 mmol, 1.2 eq) in methanol (50 mL) was added potassium carbonate (4.86 g, 35.20 mmol, 2 eq) in one portion at 0° C. under nitrogen. The mixture was stirred at 20° C. for 2 hours. The mixture was poured into ice-water (w/w=1/1, 30 mL) and stirred for 5 minutes. The aqueous phase was extracted with ethyl acetate (30 mL×4). The combined organic phase was washed with brine (20 mL×2), dried with anhydrous sodium sulfate, filtered, and concentrated in vacuum. The residue was purified by silica gel chromatography (petroleum ether/ethyl acetate=30/1 to 5/1) to afford tert-butyl 4-prop-2-ynylpiperidine-1-carboxylate (2.6 g, 11.64 mmol) as a colorless oil.

### Step 2

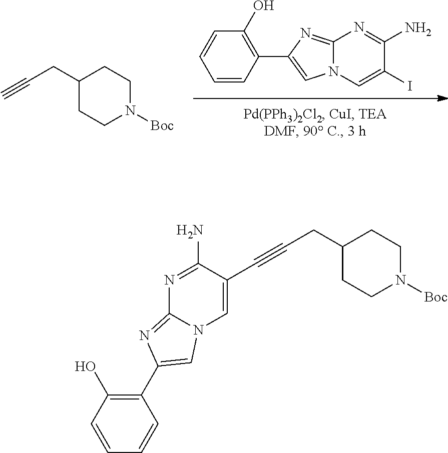 [SMILES: C#CCC1CCN(C(=O)OC(C)(C)C)CC1 ; Nc1nc2nc(-c3ccccc3O)cn2cc1I ; CC(C)(C)OC(=O)N1CCC(CC#Cc2cn3cc(-c4ccccc4O)nc3nc2N)CC1]

To a mixture of 2-(7-amino-6-iodo-imidazo[1,2-a]pyrimidin-2-yl)phenol (300 mg, 0.85 mmol, 1 eq), bis(triphenylphosphine)palladium(II)dichloride (89 mg, 0.13 mmol, 0.15 eq), cuprous iodide (32 mg, 0.17 mmol, 0.2 eq) and tert-butyl 4-prop-2-ynylpiperidine-1-carboxylate (228 mg, 1.02 mmol, 1.2 eq) in N,N-dimethylformamide (10 mL) was added triethylamine (258 mg, 2.56 mmol, 0.36 mL, 3 eq) under nitrogen. The mixture was stirred at 90° C. for 3 hours. The mixture was cooled to 20° C. and concentrated under reduced pressure at 45° C. The residue was purified by semi-preparative reverse phase HPLC (column: Phenomenex Gemini-NX C18 75*30 mm*3 um; mobile phase: [water (0.1% TFA)-ACN]; B %: 32%-42%, 7 min). Compound tert-butyl 4-[3-[7-amino-2-(2-hydroxyphenyl)imidazo[1,2-a]pyrimidin-6-yl]prop-2-ynyl]piperidine-1-carboxylate (201 mg, 0.45 mmol) was obtained as a yellow solid.

### Step 3

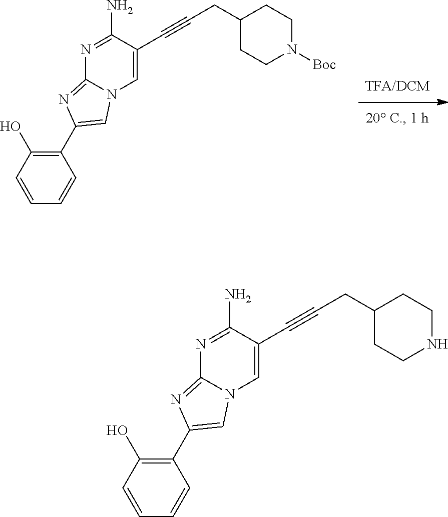 [SMILES: CC(C)(C)OC(=O)N1CCC(CC#Cc2cn3cc(-c4ccccc4O)nc3nc2N)CC1 ; Nc1nc2nc(-c3ccccc3O)cn2cc1C#CCC1CCNCC1]

A solution of tert-butyl 4-[3-[7-amino-2-(2-hydroxyphenyl)imidazo[1,2-a]pyrimidin-6-yl] prop-2-ynyl]piperidine-1-carboxylate (200 mg, 0.45 mmol, 1 eq) and trifluoroacetic acid (1.54 g, 13.51 mmol, 1 mL, 30.22 eq) in dichloromethane (5 mL) was stirred at 20° C. for 1 hours. The mixture was concentrated under reduced pressure at 45° C. The residue was purified by semi-preparative reverse phase HPLC (column: Waters Xbridge C18 150*50 mm*10 um; mobile phase: [water (10 mM NH₄HCO₃)-ACN]; B %: 15%-45%, 10 min). The product 2-[7-amino-6-[3-(4-piperidyl)prop-1-ynyl]imidazo[1,2-a]pyrimidin-2-yl]phenol (140 mg, 0.40 mmol) was obtained as a yellow solid.

### Step 4

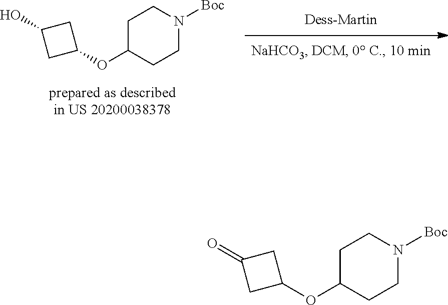 [SMILES: CC(C)(C)OC(=O)N1CCC(O[C@H]2C[C@@H](O)C2)CC1 ; CC(C)(C)OC(=O)N1CCC(OC2CC(=O)C2)CC1]

To a solution of tert-butyl 4-(3-hydroxycyclobutoxy)piperidine-1-carboxylate (1 g, 3.69 mmol, 1 eq) in dichloromethane (20 mL) was added Dess-Martin periodinane (1.88 g, 4.42 mmol, 1.2 eq) and sodium bicarbonate (496 mg, 5.90 mmol, 1.6 eq). The mixture was stirred at 0° C. for 10 minutes. The reaction mixture was diluted with water (20 mL) and extracted with ethyl acetate (50 mL×2). The combined organic phase was washed with brine (20 mL), dried with anhydrous sodium sulfate, filtered and concentrated in vacuum. The residue was purified by silica gel chromatography (petroleum ether/ethyl acetate=50/1 to 3/1). Compound tert-butyl 4-(3-oxocyclobutoxy) piperidine-1-carboxylate (800 mg, 2.97 mmol) was obtained as a white solid.

### Step 5

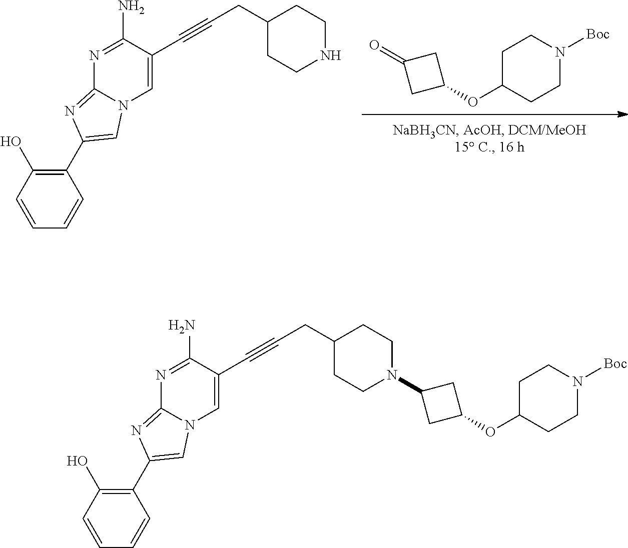

To a mixture of 2-[7-amino-6-[3-(4-piperidyl)prop-1-ynyl]imidazo[1,2-a]pyrimidin-2-yl] phenol (120 mg, 0.34 mmol, 1 eq) and tert-butyl 4-(3-oxocyclobutoxy)piperidine-1-carboxylate (111 mg, 0.41 mmol, 1.2 eq) in methanol (6 mL) and dichloromethane (2 mL) was added acetic acid (21 mg, 0.35 mmol, 1 eq) and sodium cyanoborohydride (65 mg, 1.04 mmol, 3 eq) at 15° C. under nitrogen. The mixture was stirred at 15° C. for 16 hours. The mixture was concentrated under reduced pressure at 40° C. The residue was purified by semi-preparative reverse phase HPLC (column: Phenomenex Gemini-NX C18 75*30 mm*3 um; mobile phase: [water (0.1% TFA)-ACN]; B %: 25%-35%, 7 min). Compound tert-butyl 4-[3-[4-[3-[7-amino-2-(2-hydroxyphenyl)imidazo[1,2-a]pyrimidin-6-yl]prop-2-ynyl]-1-piperidyl]cyclobutoxy]piperidine-1-carboxylate (150 mg, 0.25 mmol, 72% yield) was obtained as a white solid.

### Step 6

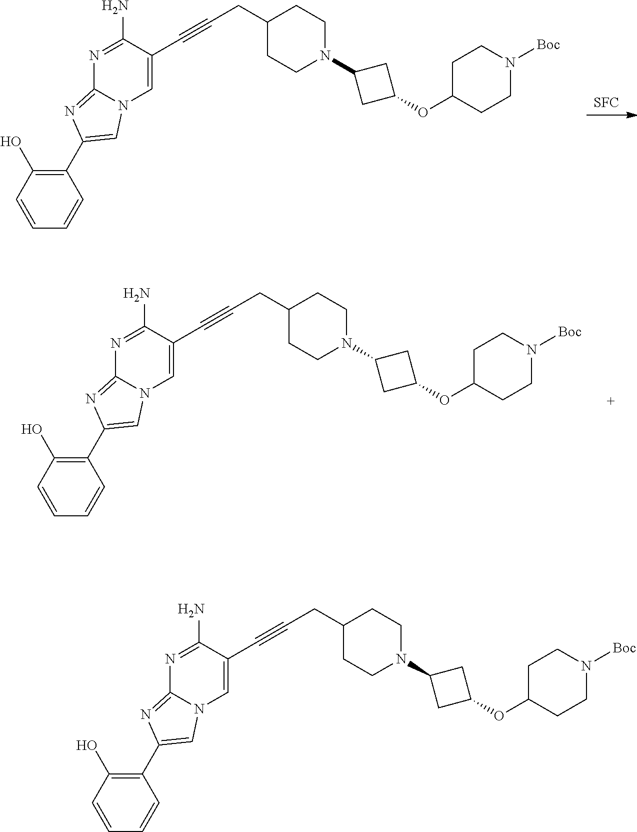 [SMILES: CC(C)(C)OC(=O)N1CCC(O[C@H]2C[C@H](N3CCC(CC#Cc4cn5cc(-c6ccccc6O)nc5nc4N)CC3)C2)CC1 ; "SFC" ; CC(C)(C)OC(=O)N1CCC(O[C@H]2C[C@@H](N3CCC(CC#Cc4cn5cc(-c6ccccc6O)nc5nc4N)CC3)C2)CC1 ; CC(C)(C)OC(=O)N1CCC(O[C@H]2C[C@H](N3CCC(CC#Cc4cn5cc(-c6ccccc6O)nc5nc4N)CC3)C2)CC1]

tert-Butyl 4-[3-[4-[3-[7-amino-2-(2-hydroxyphenyl)imidazo[1,2-a]pyrimidin-6-yl]prop-2-ynyl]-1-piperidyl]cyclobutoxy]piperidine-1-carboxylate (160 mg, 0.26 mmol, 1 eq) was separated by SFC (Column: DAICEL CHIRALPAK IC 250×30 mm, I.D., 10 um; Mobile phase: methanol (0.1% NH3H2O) in CO₂ from 50% to 50%; Flow rate: 80 mL/min; Wavelength: 220 nm). Tert-Butyl 4-((1s,3s)-3-(4-(3-(7-amino-2-(2-hydroxyphenyl)imidazo[1,2-a]pyrimidin-6-yl)prop-2-yn-1-yl)piperidin-1-yl)cyclobutoxy)piperidine-1-carboxylate (70 mg, 0.12 mmol) and tert-butyl 4-((1r,3r)-3-(4-(3-(7-amino-2-(2-hydroxyphenyl)imidazo[1,2-a]pyrimidin-6-yl)prop-2-yn-1-yl)piperidin-1-yl)cyclobutoxy)piperidine-1-carboxylate (13 mg, 0.02 mmol) were obtained as white solids.

### Step 7

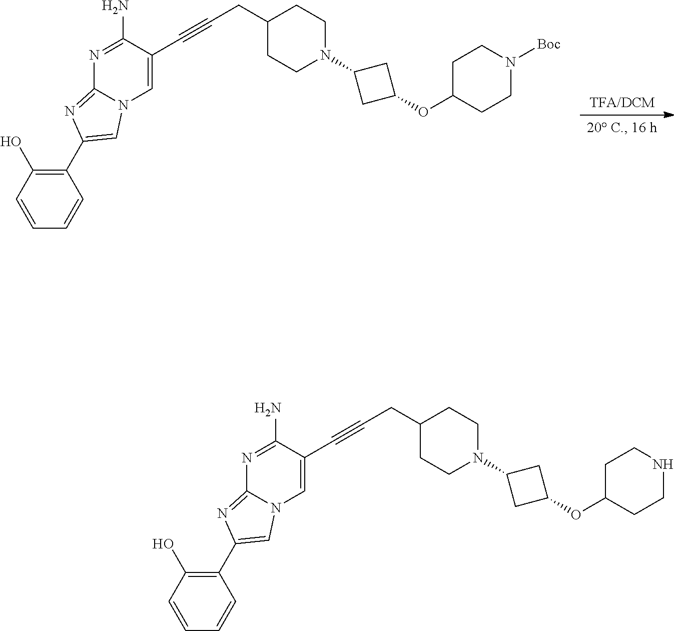 [SMILES: CC(C)(C)OC(=O)N1CCC(O[C@H]2C[C@@H](N3CCC(CC#Cc4cn5cc(-c6ccccc6O)nc5nc4N)CC3)C2)CC1 ; "20° C., 16 h" ; "TFA/DCM" ; Nc1nc2nc(-c3ccccc3O)cn2cc1C#CCC1CCN([C@H]2C[C@@H](OC3CCNCC3)C2)CC1]

A mixture of tert-butyl 4-[3-[4-[3-[7-amino-2-(2-hydroxyphenyl)imidazo[1,2-a]pyrimidin-6-yl]prop-2-ynyl]-1-piperidyl]cyclobutoxy]piperidine-1-carboxylate (70 mg, 0.12 mmol, 1 eq) and trifluoroacetic acid (0.5 mL) in dichloromethane (1 mL) was stirred at 20° C. for 16 hours. The mixture was concentrated under reduced pressure at 45° C. The crude product 2-[7-amino-6-[3-[1-[3-(4-piperidyloxy)cyclobutyl]-4-piperidyl] prop-1-ynyl]imidazo[1,2-a]pyrimidin-2-yl]phenol trifluoroacetate (58.3 mg, 0.09 mmol) was obtained as a yellow oil.

2-[7-amino-6-[3-[1-[3-(4-piperidyloxy)cyclobutyl]-4-piperidyl] prop-1-ynyl]imidazo[1,2-a]pyrimidin-2-yl]phenol was converted to the title compound according to the scheme below using procedure described for Compound 29 above.

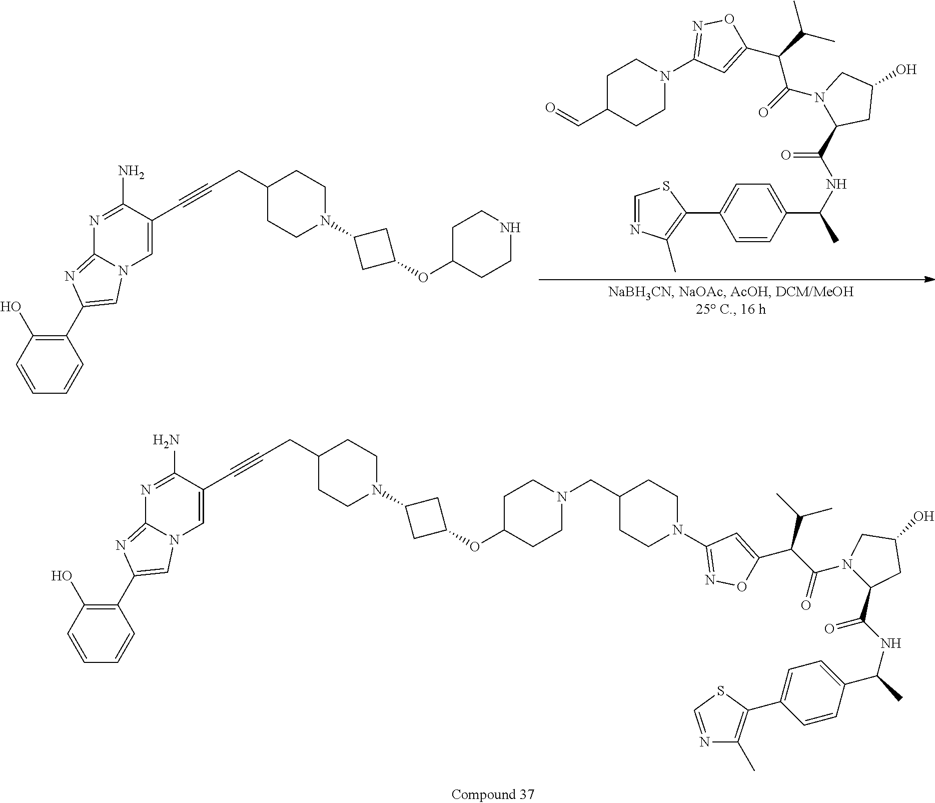 [SMILES: Nc1nc2nc(-c3ccccc3O)cn2cc1C#CCC1CCN([C@H]2C[C@@H](OC3CCNCC3)C2)CC1 ; Cc1ncsc1-c1ccc([C@H](C)NC(=O)[C@@H]2C[C@@H](O)CN2C(=O)[C@@H](c2cc(N3CCC(C=O)CC3)no2)C(C)C)cc1 ; "NaBH3CN, NaOAc, AcOH, DCM/MeOH25° C., 16 h" ; Cc1ncsc1-c1ccc([C@H](C)NC(=O)[C@@H]2C[C@@H](O)CN2C(=O)[C@@H](c2cc(N3CCC(CN4CCC(O[C@H]5C[C@@H](N6CCC(CC#Cc7cn8cc(-c9ccccc9O)nc8nc7N)CC6)C5)CC4)CC3)no2)C(C)C)cc1]

Using analogous procedures Exemplary Compound 38 was prepared.

### Exemplary Synthesis of Compound 39

### Step 1

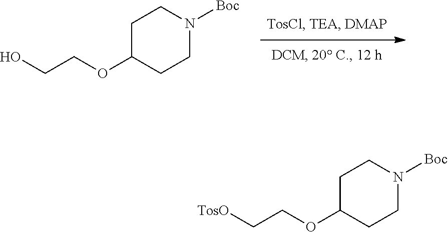

To a solution of tert-butyl 4-(2-hydroxyethoxy)piperidine-1-carboxylate (6.5 g, 26.50 mmol, 1 eq) and p-toluenesulfonyl chloride (7.58 g, 39.74 mmol, 1.5 eq) in dichloromethane (55 mL) was added triethylamine (5.36 g, 52.99 mmol, 7.4 mL, 2 eq) and dimethylaminopyridine (324 mg, 2.65 mmol, 0.1 eq). The mixture was stirred at 20° C. for 12 hours. The mixture was concentrated to afford the crude product. The residue was purified by silica gel column chromatography (petroleum ether/ethyl acetate=10/1 to 3/1). Compound tert-butyl 4-[2-(p-tolylsulfonyloxy) ethoxy]piperidine-1-carboxylate (10.45 g, 26.16 mmol) was obtained as a brown oil.

### Step 2

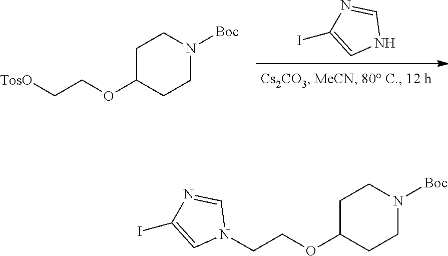

To a solution of 4-iodo-1H-imidazole (3.87 g, 19.95 mmol, 1 eq) and tert-butyl 4-[2-(p-tolylsulfonyloxy)ethoxy]piperidine-1-carboxylate (10.36 g, 25.94 mmol, 1.3 eq) in acetonitrile (100 mL) was added cesium carbonate (13.00 g, 39.90 mmol, 2 eq). The mixture was stirred at 80° C. for 12 hours. The mixture was filtered, and the filtrate was concentrated to afford the crude product which was purified by semi-preparative reverse phase (column: Phenomenex luna C18 (250*70 mm, 10 um); mobile phase: [water (0.225% FA)-ACN]; B %: 15%-60%, 20 min). Compound tert-butyl 4-[2-(4-iodoimidazol-1-yl)ethoxy]piperidine-1-carboxylate (5.7 g, 13.53 mmol) was obtained as a yellow solid.

### Step 3

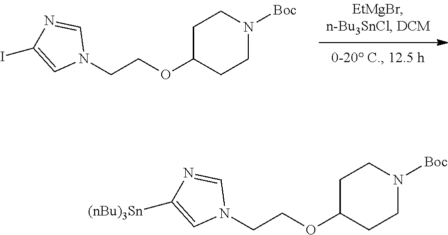

To a solution of tert-butyl 4-[2-(4-iodoimidazol-1-yl)ethoxy]piperidine-1-carboxylate (4 g, 9.50 mmol, 1 eq) in dichloromethane (150 mL) was added dropwise ethylmagnesium bromide (3 M, 6.4 mL, 2.00 eq) at 0° C. After 0.5 hours, tributyl(chloro)stannane (7.11 g, 21.84 mmol, 5.9 mL, 2.3 eq) was added, and the mixture was stirred at 20° C. for 12 hours. The mixture was poured into saturated ammonium chloride solution (200 mL) and was extracted with dichloromethane (200 mL) three times, the combined organic layers were washed with brine (300 mL) and concentrated to afford the crude product which was purified by silica gel column chromatography (dichloromethane/methanol=1/0 to 10/1). Compound tert-butyl 4-[2-(4-tributyl stannylimidazol-1-yl)ethoxy]piperidine-1-carboxylate (3.36 g) was obtained as a yellow oil.

### Step 4

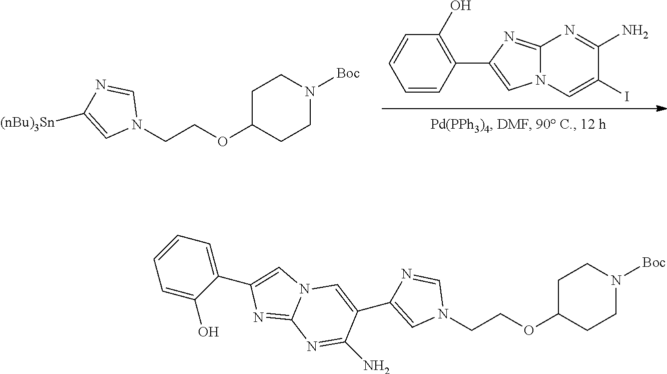

A mixture of tert-butyl 4-[2-(4-tributylstannylimidazol-1-yl)ethoxy]piperidine-1-carboxylate (3.36 g, 2.70 mmol, 47% purity, 1 eq), 2-(7-amino-6-iodo-imidazo[1,2-a]pyrimidin-2-yl) phenol (666 mg, 1.89 mmol, 0.7 eq), tetrakis(triphenylphosphine)palladium(0) (625 mg, 0.54 mmol, 0.2 eq) in N,N-dimethylformamide (30 mL) was degassed and purged with nitrogen 3 times. The mixture was then stirred at 90° C. for 12 hours under nitrogen atmosphere. The mixture was concentrated to afford the crude product which was purified by semi-preparative reverse phase (column: Phenomenex luna C18 (250*70 mm, 10 um); mobile phase: [water (0.1% trifluoroacetic acid)-ACN]; B %: 20%-50%, 20 min). Compound tert-butyl 4-[2-[4-[7-amino-2-(2-hydroxyphenyl)imidazo[1,2-a]pyrimidin-6-yl]253soxazole-1-yl]ethoxy]piperidine-1-carboxylate (150 mg) was obtained as a yellow solid.

Tert-Butyl 4-[2-[4-[7-amino-2-(2-hydroxyphenyl)imidazo[1,2-a]pyrimidin-6-yl]isoxazole-1-yl] ethoxy]piperidine-1-carboxylate was converted to the title compound according to the scheme below as described for other Examples above.

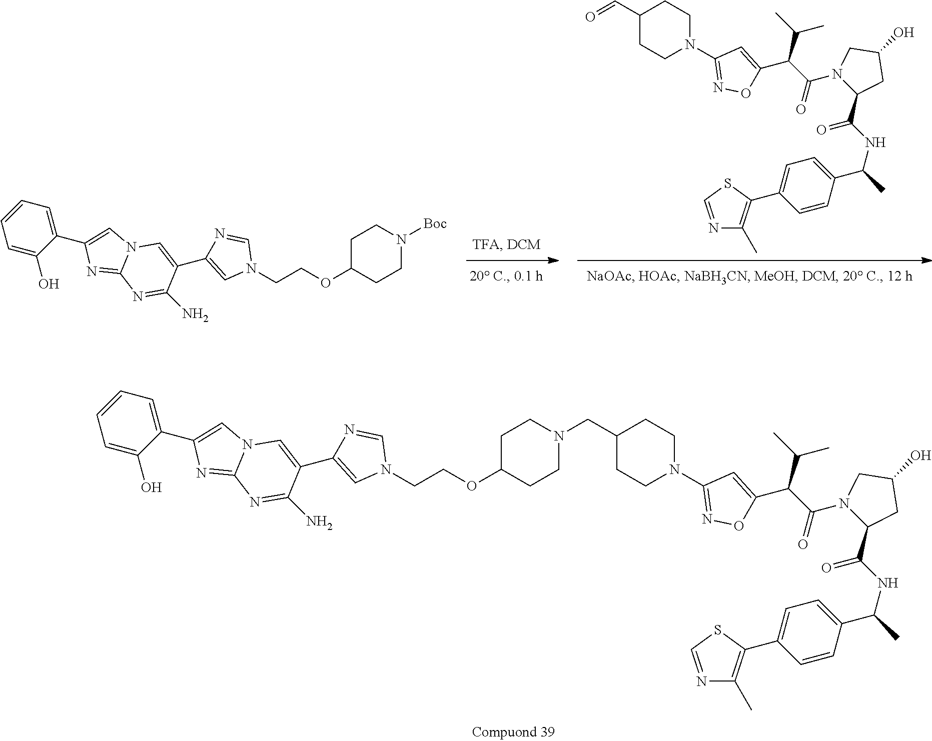 [SMILES: CC(C)(C)OC(=O)N1CCC(OCCn2cnc(-c3cn4cc(-c5ccccc5O)nc4nc3N)c2)CC1 ; Cc1ncsc1-c1ccc([C@H](C)NC(=O)[C@@H]2C[C@@H](O)CN2C(=O)[C@@H](c2cc(N3CCC(C=O)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(OCCn5cnc(-c6cn7cc(-c8ccccc8O)nc7nc6N)c5)CC4)CC3)no2)C(C)C)cc1]

### Exemplary Synthesis of Compound 40

### Step 1

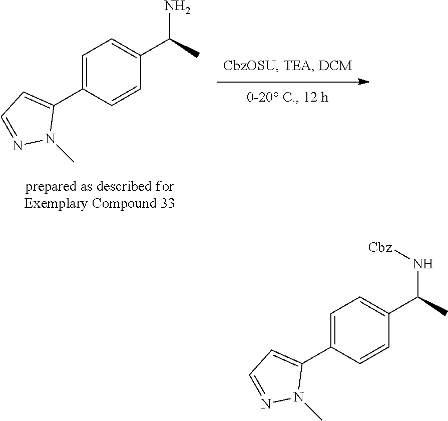 [SMILES: C[C@H](N)c1ccc(-c2ccnn2C)cc1 ; C[C@H](NC(=O)OCc1ccccc1)c1ccc(-c2ccnn2C)cc1]

To a solution of (1S)-1-[4-(2-methylpyrazol-3-yl)phenyl]ethanamine (1.6 g, 6.73 mmol, 1 eq, hydrochloride) and triethylamine (3.41 g, 33.65 mmol, 4.7 mL, 5 eq) in dichloromethane (25 mL) was added N-(benzyloxycarbonyloxy)succinimide (2.52 g, 10.10 mmol, 1.5 eq) at 0° C. The reaction solution was stirred at 20° C. for 12 hours. The reaction solution was concentrated under vacuum to remove solvents, diluted with water (30 mL) and extracted with ethyl acetate (30 mL×2). The combined organic layer was dried over anhydrous sodium sulfate, filtered, and concentrated under vacuum. The residue was purified by silica gel column chromatography (petroleum ether/ethyl acetate=10:1 to 2:1). Benzyl N-[(1S)-1-[4-(2-methylpyrazol-3-yl)phenyl]ethyl]carbamate (2.1 g, 6.26 mmol) was obtained as a white solid.

### Step 2

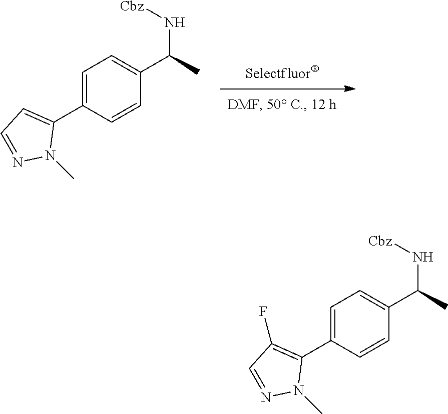 [SMILES: C[C@H](NC(=O)OCc1ccccc1)c1ccc(-c2ccnn2C)cc1 ; C[C@H](NC(=O)OCc1ccccc1)c1ccc(-c2c(F)cnn2C)cc1]

To a solution of benzyl N-[(1S)-1-[4-(2-methylpyrazol-3-yl)phenyl]ethyl]carbamate (220 mg, 0.66 mmol, 1 eq) in N,N-dimethylformamide (4 mL) was added Selectfluor® reagent (1-chloromethyl-4-fluoro-1,4-diazoniabicyclo[2.2.2]octane bis(tetrafluoroborate)) (302 mg, 0.85 mmol, 1.3 eq). The reaction solution was stirred at 50° C. for 12 hours. The reaction solution was cooled to 20° C. and was diluted with water (30 mL) and was extracted with ethyl acetate (20 mL×2). The combined organic layer was washed with brine (20 mL×4). The organic layer was dried over anhydrous sodium sulfate, filtered, and concentrated under vacuum. The residue was purified by prep-TLC (petroleum ether/ethyl acetate=1/1). Benzyl N-[(1S)-1-[4-(4-fluoro-2-methyl-pyrazol-3-yl)phenyl]ethyl] carbamate (87 mg, 0.25 mmol) was obtained as a colorless gum. The procedure was then repeated two more times, in two identical batches, to generate a total of 260 mg of the desired product.

### Step 3

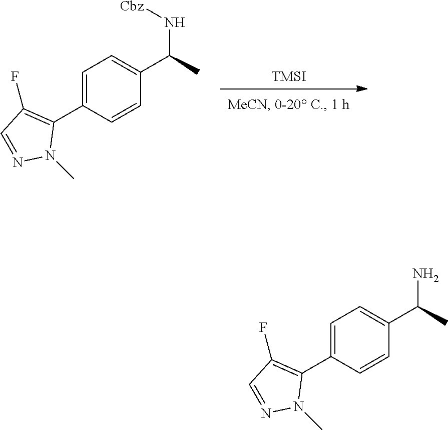 [SMILES: "TMSI" ; C[C@H](NC(=O)OCc1ccccc1)c1ccc(-c2c(F)cnn2C)cc1 ; C[C@H](N)c1ccc(-c2c(F)cnn2C)cc1]

To a solution of benzyl N-[(1S)-1-[4-(4-fluoro-2-methyl-pyrazol-3-yl)phenyl]ethyl]carbamate (260 mg, 0.74 mmol, 1 eq) in acetonitrile (5 mL) was added trimethyliodosilane (294 mg, 1.47 mmol, 0.2 mL, 2 eq) at 0° C., and the reaction solution was stirred at 20° C. for 1 hour. The reaction solution was quenched with methanol (8 mL) and concentrated under vacuum. The residue was purified by prep-HPLC (column: Phenomenex luna C18 150*25 mm*10 um; mobile phase: [water (0.1% TFA)-ACN]; B %: 1%-31%, 10 min). (1S)-1-[4-(4-fluoro-2-methyl-pyrazol-3-yl)phenyl]ethanamine trifluoroacetate (168 mg, 0.50 mmol) was obtained as a light yellow solid.

### Step 4

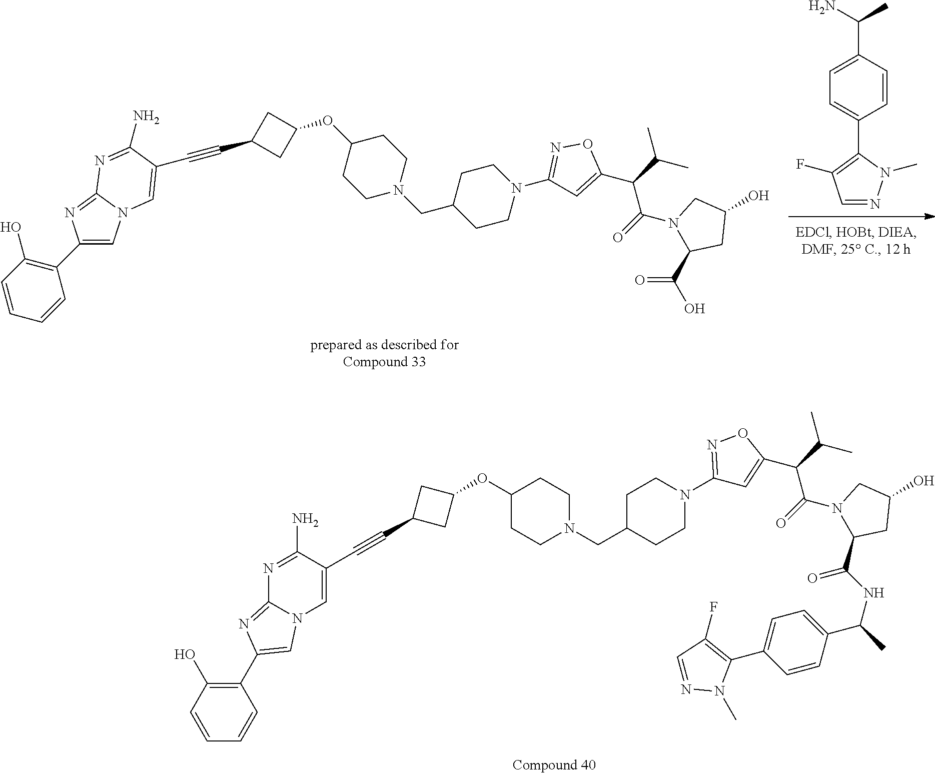 [SMILES: CC(C)[C@@H](C(=O)N1C[C@H](O)C[C@H]1C(=O)O)c1cc(N2CCC(CN3CCC(O[C@H]4C[C@H](C#Cc5cn6cc(-c7ccccc7O)nc6nc5N)C4)CC3)CC2)no1 ; C[C@H](N)c1ccc(-c2c(F)cnn2C)cc1 ; CC(C)[C@@H](C(=O)N1C[C@H](O)C[C@H]1C(=O)N[C@@H](C)c1ccc(-c2c(F)cnn2C)cc1)c1cc(N2CCC(CN3CCC(O[C@H]4C[C@H](C#Cc5cn6cc(-c7ccccc7O)nc6nc5N)C4)CC3)CC2)no1]

Procedure was carried out as described for Compound 33.

### Exemplary Synthesis of Compound 41

### Step 1

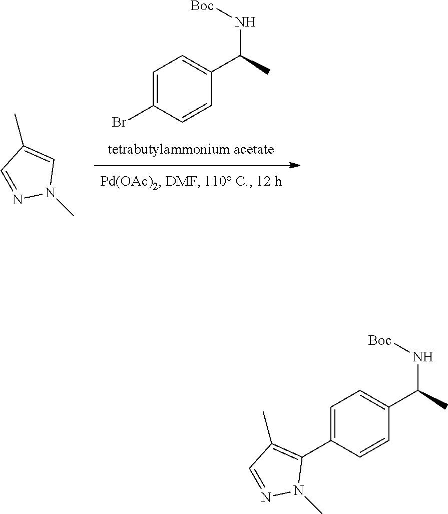

A mixture of tert-butyl N-[(1S)-1-(4-bromophenyl)ethyl]carbamate (936 mg, 3.12 mmol, 1 eq), 1,4-dimethylpyrazole (300 mg, 3.12 mmol, 1 eq), palladium(II) acetate (70 mg, 0.31 mmol, 0.1 eq), and tetrabutylammonium acetate (1.88 g, 6.24 mmol, 1.9 mL, 2 eq) in N,N-dimethylformamide (6 mL) was degassed and purged with nitrogen 3 times. The mixture was then stirred at 110° C. for 12 hours under nitrogen atmosphere. The reaction mixture was filtered. The residue was purified by prep-HPLC (column: Phenomenex luna C18 150*40 mm*15 um; mobile phase: [water (0.1% TFA)-ACN]; B %: 35%-65%, 10 min). Compound tert-butyl N-[(1S)-1-[4-(2,4-dimethylpyrazol-3-yl)phenyl]ethyl]carbamate (300 mg, 0.95 mmol) was obtained as a yellow gum.

### Step 2

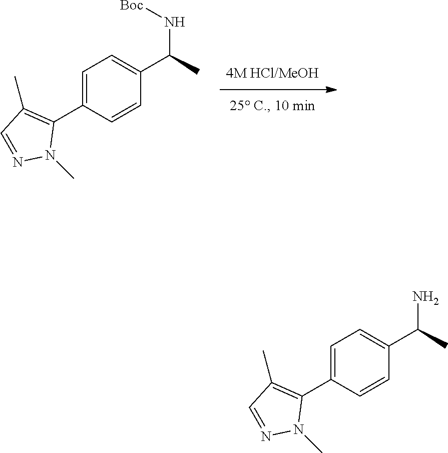 [SMILES: Cc1cnn(C)c1-c1ccc([C@H](C)NC(=O)OC(C)(C)C)cc1 ; Cc1cnn(C)c1-c1ccc([C@H](C)N)cc1]

A solution of tert-butyl N-[(1S)-1-[4-(2,4-dimethylpyrazol-3-yl)phenyl]ethyl]carbamate (300 mg, 0.95 mmol, 1 eq) in hydrochloric acid/methanol (4 M, 10 mL, 42 eq) was stirred at 25° C. for 10 minutes. The reaction mixture was concentrated under reduced pressure. The crude product was used in the next step without further purification. Compound (1S)-1-[4-(2,4-dimethylpyrazol-3-yl)phenyl]ethanamine hydrochloride (230 mg, 0.91 mmol) was obtained as a yellow gum.

### Step 3

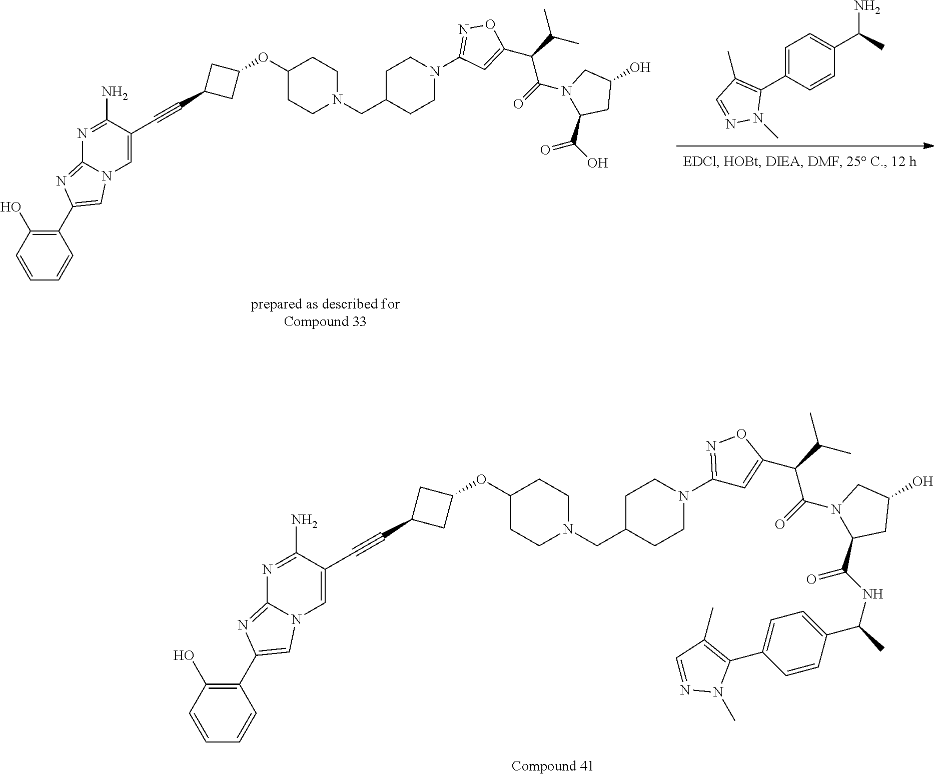 [SMILES: CC(C)[C@@H](C(=O)N1C[C@H](O)C[C@H]1C(=O)O)c1cc(N2CCC(CN3CCC(O[C@H]4C[C@H](C#Cc5cn6cc(-c7ccccc7O)nc6nc5N)C4)CC3)CC2)no1 ; Cc1cnn(C)c1-c1ccc([C@H](C)N)cc1 ; Cc1cnn(C)c1-c1ccc([C@H](C)NC(=O)[C@@H]2C[C@@H](O)CN2C(=O)[C@@H](c2cc(N3CCC(CN4CCC(O[C@H]5C[C@H](C#Cc6cn7cc(-c8ccccc8O)nc7nc6N)C5)CC4)CC3)no2)C(C)C)cc1]

Procedure was carried out as described for Compound 33.

### Exemplary Synthesis of Compound 42

### Step 1

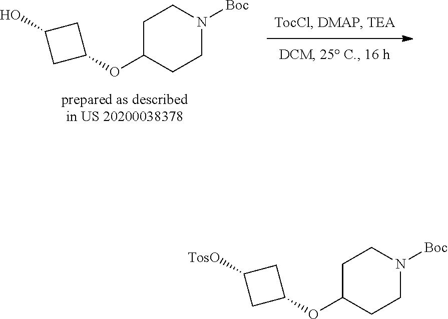

To a mixture of tert-butyl 4-(3-hydroxycyclobutoxy)piperidine-1-carboxylate (1 g, 3.69 mmol, 1 eq) [prepared as described in US 20200038378], triethylamine (1.12 g, 11.06 mmol, 1.5 mL, 3 eq) and 4-dimethylaminopyridine (225 mg, 1.84 mmol, 0.5 eq) in dichloromethane (15 mL) was added p-toluenesulfonyl chloride (843 mg, 4.42 mmol, 1.2 eq) at 0° C. under nitrogen. The mixture was stirred at 25° C. for 16 hours. The mixture was poured into ice-water (w/w=1/1, 10 mL) and stirred for 2 minutes. The aqueous phase was extracted with dichloromethane (10 mL×2). The combined organic phase was washed with brine (10 mL), dried with anhydrous sodium sulfate, filtered, and concentrated in vacuum. The residue was purified by silica gel chromatography (petroleum ether/ethyl acetate=20/1 to 3/1) to afford tert-butyl 4-[3-(p-tolylsulfonyloxy)cyclobutoxy]piperidine-1-carboxylate (1.36 g, 3.20 mmol) as a colorless oil.

### Step 2

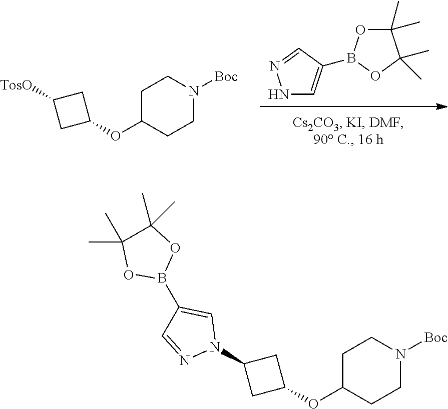

To a mixture of 4-(4,4,5,5-tetramethyl-1,3,2-dioxaborolan-2-yl)-1H-pyrazole (400 mg, 2.06 mmol, 1 eq), potassium iodide (171 mg, 1.03 mmol, 0.5 eq) and tert-butyl 4-[3-(p-tolylsulfonyloxy)cyclobutoxy]piperidine-1-carboxylate (877 mg, 2.06 mmol, 1 eq) in N,N-dimethylformamide (10 mL) was added cesium carbonate (1.34 g, 4.12 mmol, 2 eq). The mixture was stirred at 90° C. for 16 hours. The mixture was cooled to 20° C. and poured into ice-water (w/w=1/1, 20 mL) and stirred for 5 minutes. The aqueous phase was extracted with ethyl acetate (30 mL×5). The combined organic phase was washed with brine (20 mL×2), dried with anhydrous sodium sulfate, filtered, and concentrated in vacuum. The residue was purified by silica gel chromatography (petroleum ether/ethyl acetate=10/1 to 3/1) to afford tert-butyl 4-[3-[4-(4,4,5,5-tetramethyl-1,3,2-dioxaborolan-2-yl)pyrazol-1-yl]cyclobutoxy] piperidine-1-carboxylate (510 mg, 1.14 mmol) as a yellow solid.

### Step 3

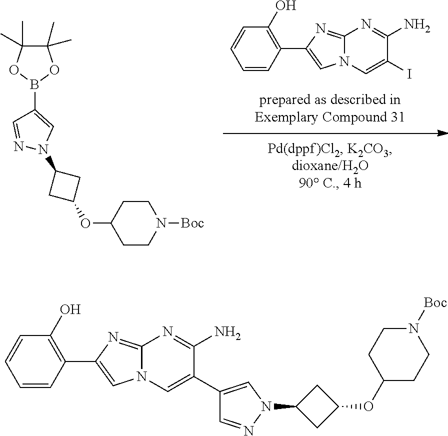 [SMILES: CC(C)(C)OC(=O)N1CCC(O[C@H]2C[C@H](n3cc(B4OC(C)(C)C(C)(C)O4)cn3)C2)CC1 ; Nc1nc2nc(-c3ccccc3O)cn2cc1I ; CC(C)(C)OC(=O)N1CCC(O[C@H]2C[C@H](n3cc(-c4cn5cc(-c6ccccc6O)nc5nc4N)cn3)C2)CC1]

To a mixture of 2-(7-amino-6-iodo-imidazo[1,2-a]pyrimidin-2-yl)phenol (300 mg, 0.85 mmol, 1 eq), potassium carbonate (353 mg, 2.56 mmol, 3 eq) and tert-butyl 4-[3-[4-(4,4,5,5-tetramethyl-1,3,2-dioxaborolan-2-yl)pyrazol-1-yl]cyclobutoxy]piperidine-1-carboxylate (457 mg, 1.02 mmol, 1.2 eq) in dioxane (12 mL) and water (2 mL) was added [1,1′-bis(diphenylphosphino)ferrocene]dichloropalladium(II) (62 mg, 0.09 mmol, 0.1 eq). The mixture was stirred at 90° C. for 4 hours. The mixture was cooled to 20° C. and concentrated under reduced pressure at 40° C. The residue was purified by semi-preparative reverse phase HPLC (column: Phenomenex luna C18 150*40 mm*15 um; mobile phase: [water (0.1% TFA)-ACN]; B %: 22%-52%, 10 min) to afford tert-butyl 4-[3-[4-[7-amino-2-(2-hydroxy phenyl)imidazo[1,2-a]pyrimidin-6-yl]pyrazol-1-yl]cyclobutoxy]piperidine-1-carboxylate (230 mg, 0.42 mmol) as a yellow solid.

### Step 4

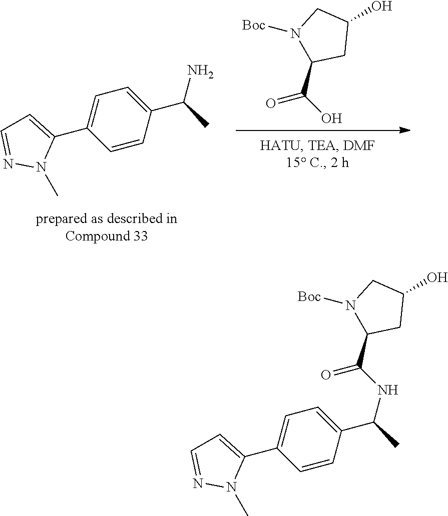 [SMILES: C[C@H](N)c1ccc(-c2ccnn2C)cc1 ; CC(C)(C)OC(=O)N1C[C@H](O)C[C@H]1C(=O)O ; C[C@H](NC(=O)[C@@H]1C[C@@H](O)CN1C(=O)OC(C)(C)C)c1ccc(-c2ccnn2C)cc1]

To a mixture of (1S)-1-[4-(2-methylpyrazol-3-yl)phenyl]ethanamine (3.3 g, 16 mmol, 1 eq, hydrochloride), triethylamine (8.3 g, 82.03 mmol, 11 mL, 5 eq) and (2S,4R)-1-tert-butoxycarbonyl-4-hydroxy-pyrrolidine-2-carboxylic acid (3.8 g, 16 mmol, 1 eq) in N,N-dimethylformamide (80 mL) was added O-(7-azabenzotriazol-1-yl)-N,N,N,N-tetramethyluronium hexafluorophosphate (8.1 g, 21 mmol, 1 eq) under nitrogen, and the mixture was stirred at 15° C. for 2 hours. The mixture was poured into ethyl acetate (800 mL), the combined organic phase was washed with brine (50 mL), dried with anhydrous sodium sulfate, filtered and concentrated in vacuum. The residue was purified by prep-HPLC (FA condition, column: Phenomenex luna C18 250*50 mm*10 um; mobile phase: [water (0.225% FA)-ACN]; B %: 15ACN %-45ACN %, 30 min). tert-Butyl (2S,4R)-4-hydroxy-2-[[(1S)-1-[4-(2-methylpyrazol-3-yl)phenyl]ethyl]carbamoyl]pyrrolidine-1-carboxylate (5.7 g, 13 mmol) was obtained as a yellow solid.

### Step 5

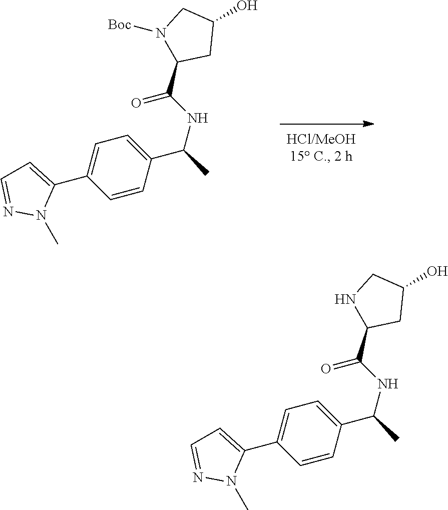 [SMILES: C[C@H](NC(=O)[C@@H]1C[C@@H](O)CN1C(=O)OC(C)(C)C)c1ccc(-c2ccnn2C)cc1 ; C[C@H](NC(=O)[C@@H]1C[C@@H](O)CN1)c1ccc(-c2ccnn2C)cc1]

A solution of tert-butyl (2S,4R)-4-hydroxy-2-[[(1S)-1-[4-(4-methylthiazol-5-yl)phenyl]ethyl] carbamoyl]pyrrolidine-1-carboxylate (5.7 g, 13.00 mmol, 1 eq) in hydrochloric acid/methanol (4 M, 50 mL, 15 eq) was stirred at 15° C. for 1 hour. The mixture was concentrated in vacuum at 45° C. Crude (2S,4R)-4-hydroxy-N-[(1S)-1-[4-(4-methylthiazol-5-yl)phenyl]ethyl]pyrrolidine-2-carboxamide hydrochloride (4.8 g) was obtained as a yellow solid and used without purification.

### Step 6

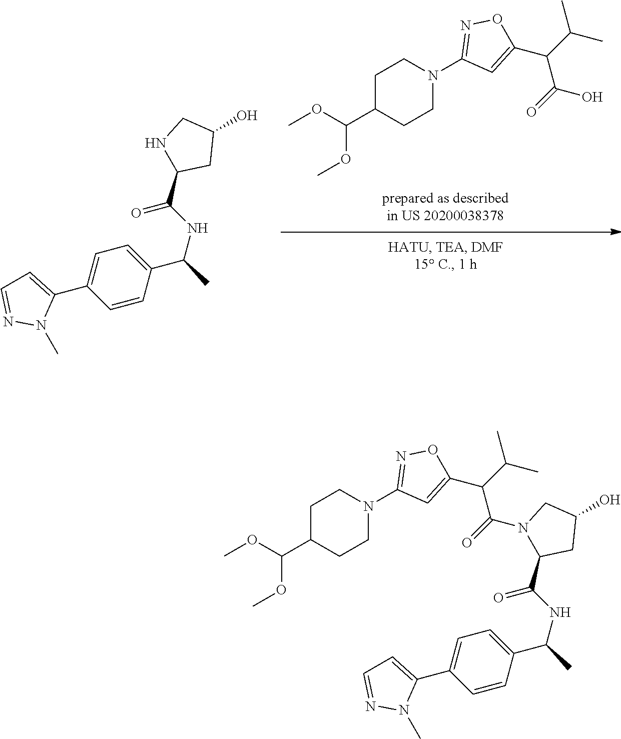 [SMILES: C[C@H](NC(=O)[C@@H]1C[C@@H](O)CN1)c1ccc(-c2ccnn2C)cc1 ; COC(OC)C1CCN(c2cc(C(C(=O)O)C(C)C)on2)CC1 ; COC(OC)C1CCN(c2cc(C(C(=O)N3C[C@H](O)C[C@H]3C(=O)N[C@@H](C)c3ccc(-c4ccnn4C)cc3)C(C)C)on2)CC1]

To a mixture of 2-[3-[4-(dimethoxymethyl)-1-piperidyl]isoxazole-5-yl]-3-methyl-butanoic acid (3.6 g, 11.00 mmol, 1 eq), triethylamine (4.4 g, 44.00 mmol, 6 mL, 4 eq) and (2S,4R)-4-hydroxy-N-[(1S)-1-[4-(2-methylpyrazol-3-yl)phenyl]ethyl]pyrrolidine-2-carboxamide (3.8 g, 12.00 mmol, 1 eq, hydrochloride) in N,N-dimethylformamide (80 mL) was added O-(7-azabenzotriazol-1-yl)-N,N,N,N-tetramethyluronium hexafluorophosphate (6.3 g, 16.00 mmol, 1 eq) under nitrogen, and the mixture was stirred at 15° C. for 1 hour. The mixture was poured into ice-water (w/w=1/1, 200 mL) and stirred for 5 minute, the aqueous phase was extracted with ethyl acetate (300 mL×6), the combined organic phase was washed with brine (100 mL), dried with anhydrous sodium sulfate, filtered and concentrated in vacuum. The residue was purified by prep-HPLC (TFA condition; column: Phenomenex luna C18 (250*70 mm, 10 um); mobile phase: [water (0.1% TFA)-ACN]; B %: 30%-60%, 20 min). Compound (2S,4R)-1-[2-[3-[4-(dimethoxymethyl)-1-piperidyl]isoxazole-5-yl]-3-methyl-butanoyl]-4-hydroxy-N-[(1S)-1-[4-(2-methylpyrazol-3-yl)phenyl]ethyl]pyrrolidine-2-carboxamide (2.8 g, 4.5 mmol) was obtained as a yellow solid.

### Step 7

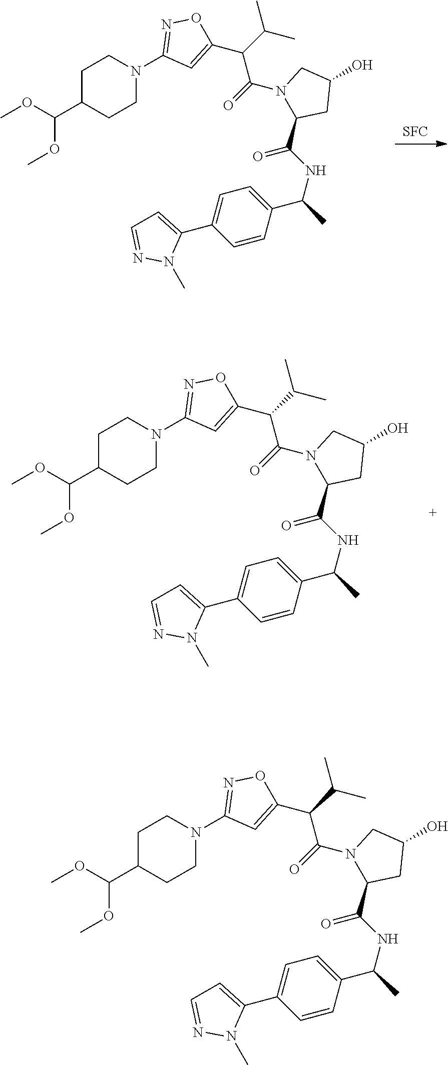 [SMILES: COC(OC)C1CCN(c2cc(C(C(=O)N3C[C@H](O)C[C@H]3C(=O)N[C@@H](C)c3ccc(-c4ccnn4C)cc3)C(C)C)on2)CC1 ; "SFC" ; COC(OC)C1CCN(c2cc([C@@H](C(=O)N3C[C@H](O)C[C@H]3C(=O)N[C@@H](C)c3ccc(-c4ccnn4C)cc3)C(C)C)on2)CC1 ; COC(OC)C1CCN(c2cc([C@H](C(=O)N3C[C@H](O)C[C@H]3C(=O)N[C@@H](C)c3ccc(-c4ccnn4C)cc3)C(C)C)on2)CC1]

The compound (2S,4R)-1-[2-[3-[4-(dimethoxymethyl)-1-piperidyl]260soxazole-5-yl]-3-methyl-butanoyl]-4-hydroxy-N-[(1S)-1-[4-(2-methylpyrazol-3-yl)phenyl]ethyl]pyrrolidine-2-carboxamide (2.4 g, 3.80 mmol) was separated by SFC (Instrument: Waters 80Q; Column: DAICEL CHIRALPAK OD 250×30 mm, I.D., 10 um; Mobile Phase: 50% IPA (0.1% NH3·H2O) in supercritical CO₂; Flow Rate: 80 g/min; Cycle Time: 3.2 min; total time: 130 min; Single injection volume: 3.0 ml; Pressure: 100 bar). (2S,4R)-1-[(2S)-2-[3-[4-(Dimethoxymethyl)-1-piperidyl]isoxazole-5-yl]-3-methyl-butanoyl]-4-hydroxy-N-[(1S)-1-[4-(2-methylpyrazol-3-yl)phenyl]ethyl]pyrrolidine-2-carboxamide (0.7 g, 1.10 mmol) and (2S,4R)-1-[(2R)-2-[3-[4-(Dimethoxymethyl)-1-piperidyl]isoxazole-5-yl]-3-methyl-butanoyl]-4-hydroxy-N-[(1S)-1-[4-(2-methylpyrazol-3-yl)phenyl]ethyl]pyrrolidine-2-carboxamide (1.6 g, 2.50 mmol) were obtained as white solids.

Tert-Butyl 4-[3-[4-[7-amino-2-(2-hydroxy phenyl)imidazo[1,2-a]pyrimidin-6-yl]pyrazol-1-yl]cyclobutoxy]piperidine-1-carboxylate was converted to the title compound according to the scheme below using procedures described for Exemplary Compound 28.

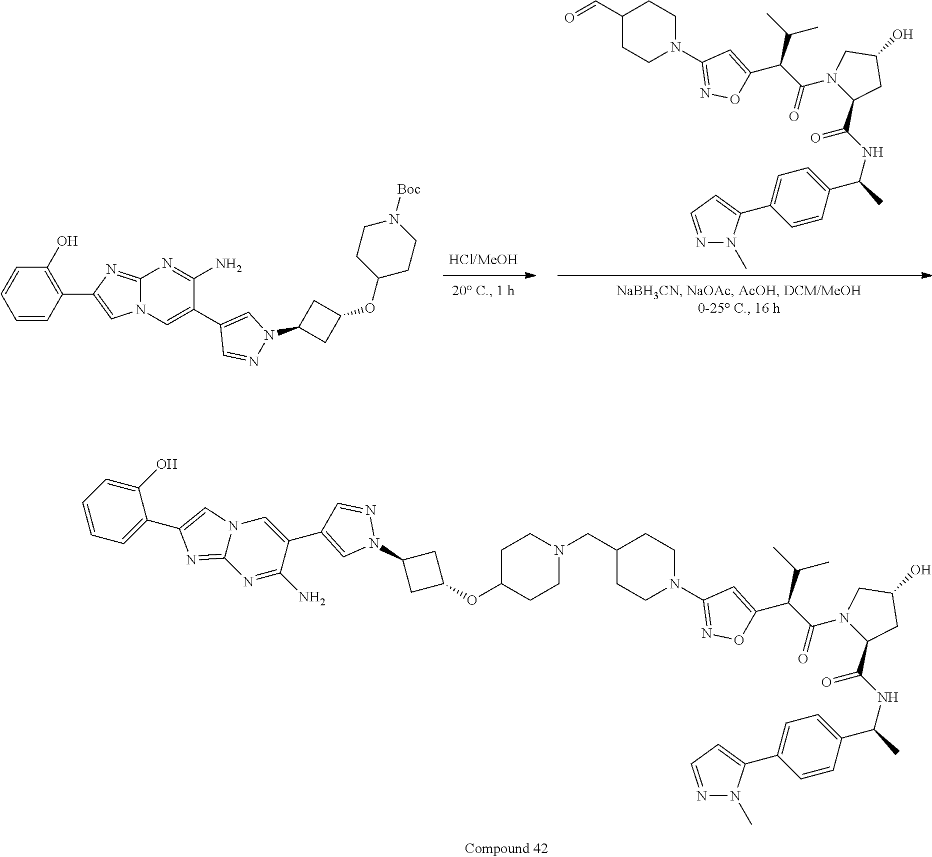 [SMILES: CC(C)(C)OC(=O)N1CCC(O[C@H]2C[C@H](n3cc(-c4cn5cc(-c6ccccc6O)nc5nc4N)cn3)C2)CC1 ; CC(C)[C@@H](C(=O)N1C[C@H](O)C[C@H]1C(=O)N[C@@H](C)c1ccc(-c2ccnn2C)cc1)c1cc(N2CCC(C=O)CC2)no1 ; CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](n5cc(-c6cn7cc(-c8ccccc8O)nc7nc6N)cn5)C4)CC3)CC2)no1]

### Exemplary Synthesis of Compound 43

### Step 1

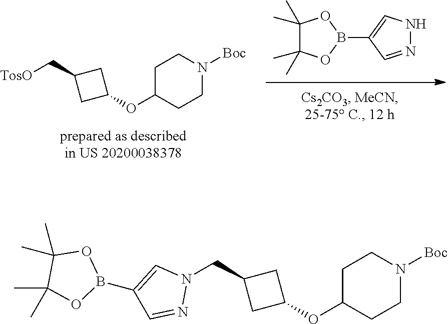

To a mixture of tert-butyl 4-[3-(p-tolylsulfonyloxymethyl)cyclobutoxy]piperidine-1-carboxylate (500 mg, 1.14 mmol, 1 eq) and 4-(4,4,5,5-tetramethyl-1,3,2-dioxaborolan-2-yl)-1H-pyrazole (882 mg, 4.55 mmol, 4 eq) in acetonitrile (10 mL) was added cesium carbonate (1.48 g, 4.55 mmol, 4 eq) under nitrogen. The mixture was heated to 75° C. and stirred for 12 hours. The reaction mixture was diluted with water (200 mL) and extracted with ethyl acetate (200 mL×2). The combined organic phase was washed with saturated brine (200 mL), dried with anhydrous sodium sulfate, filtered, and concentrated under vacuum. The residue was purified by prep-HPLC (column: Phenomenex luna C18 150*40 mm*15 um; mobile phase: [water (0.225% FA)-ACN]; B %: 52%-82%, 10 min). Compound tert-butyl 4-[3-[[4-(4,4,5,5-tetramethyl-1,3,2-dioxaborolan-2-yl)pyrazol-1-yl]methyl]cyclobutoxy]piperidine-1-carboxylate (300 mg, 0.65 mmol) was obtained as a yellow oil.

### Step 2

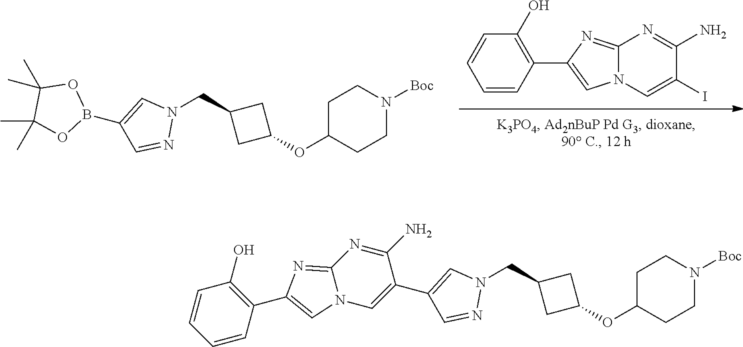 [SMILES: CC(C)(C)OC(=O)N1CCC(O[C@H]2C[C@H](Cn3cc(B4OC(C)(C)C(C)(C)O4)cn3)C2)CC1 ; Nc1nc2nc(-c3ccccc3O)cn2cc1I ; CC(C)(C)OC(=O)N1CCC(O[C@H]2C[C@H](Cn3cc(-c4cn5cc(-c6ccccc6O)nc5nc4N)cn3)C2)CC1]

A mixture of 2-(7-amino-6-iodo-imidazo[1,2-a]pyrimidin-2-yl)phenol (96 mg, 0.27 mmol, 1 eq) tert-butyl 4-[3-[[4-(4,4,5,5-tetramethyl-1,3,2-dioxaborolan-2-yl)pyrazol-1-yl]methyl] cyclobutoxy]piperidine-1-carboxylate (125 mg, 0.27 mmol, 1 eq), (di(1-adamantyl)-butylphosphine)-2-(2′-amino-1,1′-biphenyl)]palladium(II) methanesulfonate, (cataCXium-A-Pd-G3) (19 mg, 0.027 mmol, 0.1 eq) and potassium phosphate (1.5 M, 2 mL, 11 eq) in dioxane (10 mL) was degassed and purged with nitrogen 3 times, and then the mixture was stirred at 90° C. for 12 hours under nitrogen atmosphere. The reaction mixture was concentrated under reduced pressure to give a residue. The residue was purified by prep-HPLC (column: Waters Xbridge 150*25 mm*5 um; mobile phase: [water (10 mM NH₄HCO₃)-ACN]; B %: 40%-70%, 10 min). Compound tert-butyl 4-[3-[[4-[7-amino-2-(2-hydroxyphenyl)imidazo[1,2-a]pyrimidin-6-yl]pyrazol-1-yl]methyl]cyclobutoxy]piperidine-1-carboxylate (85 mg, 0.15 mmol) was obtained as a white solid.

Tert-Butyl 4-[3-[[4-[7-amino-2-(2-hydroxyphenyl)imidazo[1,2-a]pyrimidin-6-yl]pyrazol-1-yl]methyl]cyclobutoxy]piperidine-1-carboxylate was converted to the title compound according to the scheme below using procedures described for other examples above.

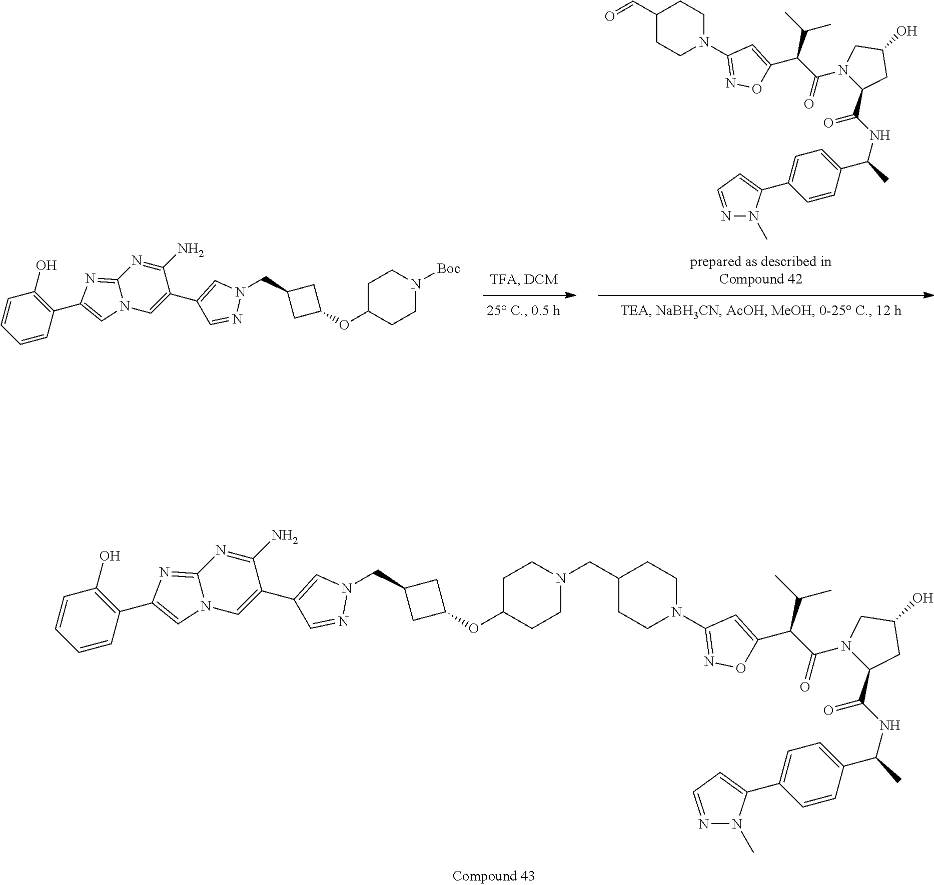 [SMILES: CC(C)[C@@H](C(=O)N1C[C@H](O)C[C@H]1C(=O)N[C@@H](C)c1ccc(-c2ccnn2C)cc1)c1cc(N2CCC(C=O)CC2)no1 ; CC(C)(C)OC(=O)N1CCC(O[C@H]2C[C@H](Cn3cc(-c4cn5cc(-c6ccccc6O)nc5nc4N)cn3)C2)CC1 ; CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](Cn5cc(-c6cn7cc(-c8ccccc8O)nc7nc6N)cn5)C4)CC3)CC2)no1]

### Synthesis of Compound 44

### Step 1

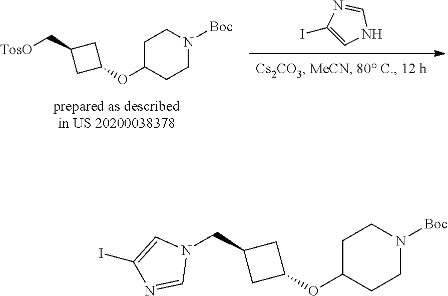

To a solution of 4-iodo-1H-imidazole (3.4 g, 17.53 mmol, 1 eq) and tert-butyl 4-[3-(p-tolylsulfonyloxymethyl)cyclobutoxy]piperidine-1-carboxylate (8.48 g, 19.28 mmol, 1.1 eq) in acetonitrile (100 mL) was added cesium carbonate (11.42 g, 35.06 mmol, 2 eq). The mixture was stirred at 80° C. for 12 hours. The reaction mixture was filtered and concentrated under reduced pressure. The residue was purified by prep-HPLC (column: Phenomenex luna C18 (250*70 mm, 10 um); mobile phase: [water (0.225% FA)-ACN]; B %: 35%-65%, 20 min). Compound tert-butyl 4-[3-[(4-iodoimidazol-1-yl)methyl]cyclobutoxy]piperidine-1-carboxylate (5 g, 10.84 mmol) was obtained as a colorless oil.

### Step 2

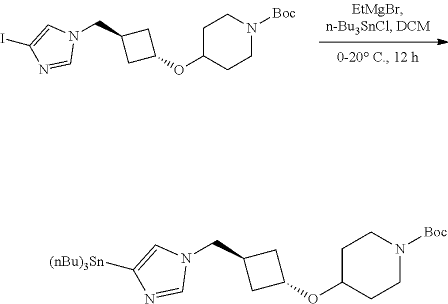

To a solution of tert-butyl 4-[3-[(4-iodoimidazol-1-yl)methyl]cyclobutoxy]piperidine-1-carboxylate (2 g, 4.34 mmol, 1 eq) in dichloromethane (80 mL) was added ethylmagnesium bromide (3 M, 2.9 mL, 2.0 eq) at 0° C. The mixture was stirred for 0.5 h, and tributyl(chloro)stannane (3.53 g, 10.84 mmol, 2.9 mL, 2.5 eq) was added. The mixture was stirred at 20° C. for 12 hours. The mixture was poured into saturated ammonium chloride solution (100 mL) and extracted with dichloromethane (50 mL×3), the combined organic layers were washed with brine (100 mL) and concentrated to afford the crude product. The crude product was purified by silica gel column chromatography (petroleum ether/ethyl acetate=20/1 to 0/1). Compound tert-butyl 4-[3-[(4-tributylstannylimidazol-1-yl)methyl]cyclobutoxy]piperidine-1-carboxylate (1.1 g, crude) was obtained as a yellow oil.

### Step 3

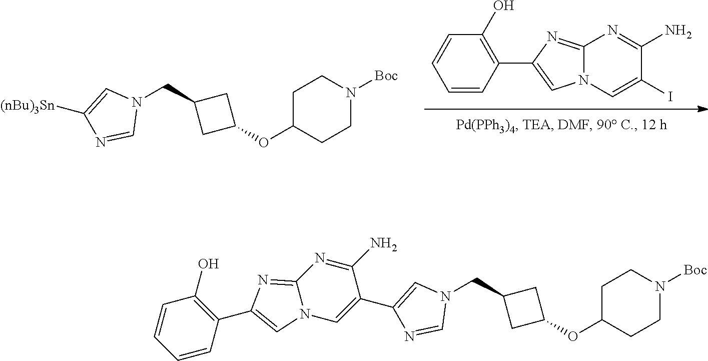

To a solution of tert-butyl 4-[3-[(4-tributylstannylimidazol-1-yl)methyl]cyclobutoxy] piperidine-1-carboxylate (1 g, 1.60 mmol, 1 eq), 2-(7-amino-6-iodo-imidazo[1,2-a]pyrimidin-2-yl)phenol (282 mg, 0.8 mmol, 0.5 eq) in N,N-dimethylformamide (10 mL) was added tetrakis[triphenylphosphine]palladium(0) (185 mg, 0.16 mmol, 0.1 eq) and triethylamine (324 mg, 3.20 mmol, 2 eq). The mixture was stirred at 90° C. for 12 hour under nitrogen. The reaction mixture was diluted with water (100 mL) and extracted with ethyl acetate (50 mL×3). The combined organic layers were washed with brine (100 mL), dried over anhydrous sodium sulfate, filtered and concentrated under reduced pressure. The residue was purified by prep-HPLC (column: Phenomenex luna C18 (250*70 mm, 15 um); mobile phase: [water (0.225% FA)-ACN]; B %: 27ACN %-57ACN %, 17 min). Compound tert-butyl 4-[3-[[4-[7-amino-2-(2-hydroxyphenyl)imidazo[1,2-a]pyrimidin-6-yl]imidazol-1-yl]methyl]cyclobutoxy]piperidine-1-carboxylate (160 mg, 0.22 mmol) was obtained as a white solid.

tert-Butyl 4-[3-[[4-[7-amino-2-(2-hydroxyphenyl)imidazo[1,2-a]pyrimidin-6-yl]imidazol-1-yl]methyl]cyclobutoxy]piperidine-1-carboxylate was converted to the title compound according to the scheme below as described for Compound 39 and other Examples above.

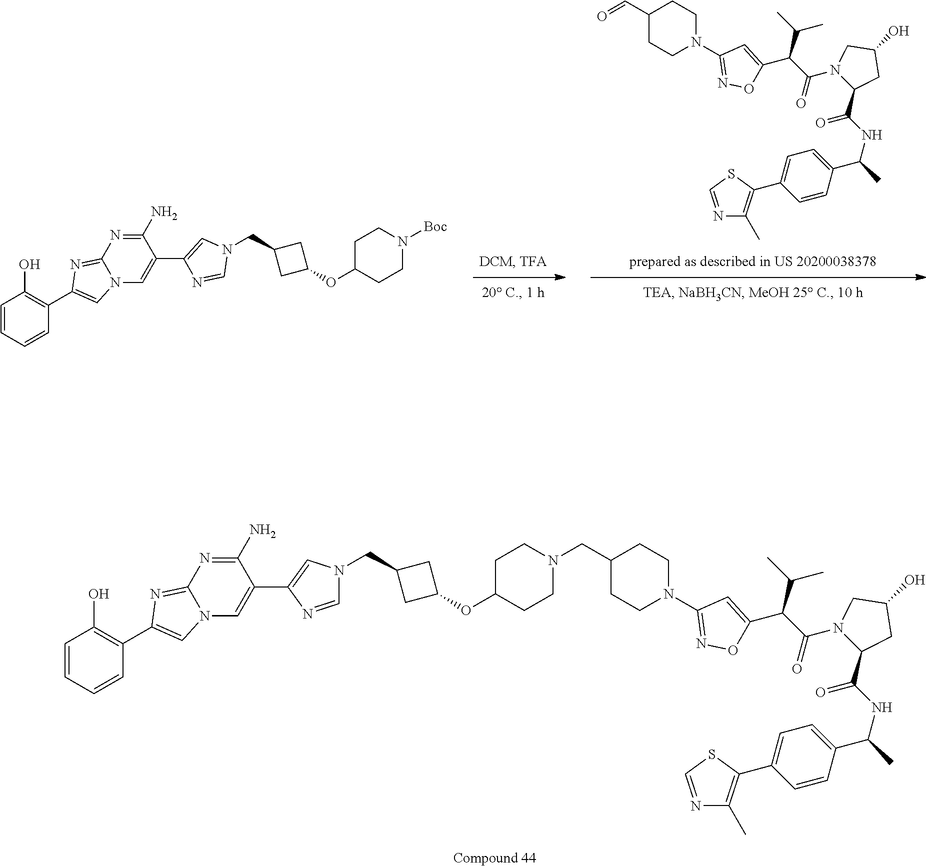 [SMILES: CC(C)(C)OC(=O)N1CCC(O[C@H]2C[C@H](Cn3cnc(-c4cn5cc(-c6ccccc6O)nc5nc4N)c3)C2)CC1 ; Cc1ncsc1-c1ccc([C@H](C)NC(=O)[C@@H]2C[C@@H](O)CN2C(=O)[C@@H](c2cc(N3CCC(C=O)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Cn6cnc(-c7cn8cc(-c9ccccc9O)nc8nc7N)c6)C5)CC4)CC3)no2)C(C)C)cc1]

### Exemplary Synthesis of Compound 45

### Step 1

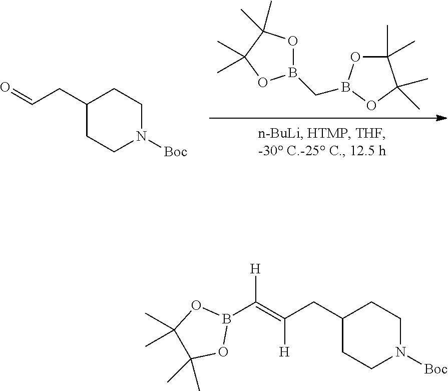

To a solution of 2,2,6,6-tetramethylpiperidine (746 mg, 5.28 mmol, 1.2 eq) in tetrahydrofuran (8 mL) was added dropwise n-butyllithium (2.5 M, 2.1 mL, 1.2 eq) at −30° C., and the mixture was stirred for 0.5 hours at −30° C. under nitrogen. Then, a solution of 4,4,5,5-tetramethyl-2-[(4,4,5,5-tetramethyl-1,3,2-dioxaborolan-2-yl)methyl]-1,3,2-dioxaborolane (1.41 g, 5.28 mmol, 1.2 eq) in tetrahydrofuran (4 mL) and a solution of tert-butyl 4-(2-oxoethyl)piperidine-1-carboxylate (1 g, 4.40 mmol, 1 eq) in tetrahydrofuran (4 mL) were added dropwise at −78° C., and the mixture was slowly warmed to 25° C. and stirred for 12 hours at 25° C. 200 mL of water was added to the mixture, and the mixture was extracted with ethyl acetate (50 mL×3). The combined organic phase was washed with brine (30 mL×2), dried with anhydrous sodium sulfate, filtered, and concentrated in vacuum. The residue was purified by silica gel chromatography (petroleum ether/ethyl acetate=50/1 to 25/1). Compound tert-butyl 4-[3-(4,4,5,5-tetramethyl-1,3,2-dioxaborolan-2-yl)allyl]piperidine-1-carboxylate (1 g, 2.85 mmol) was obtained as a white solid.

### Step 2

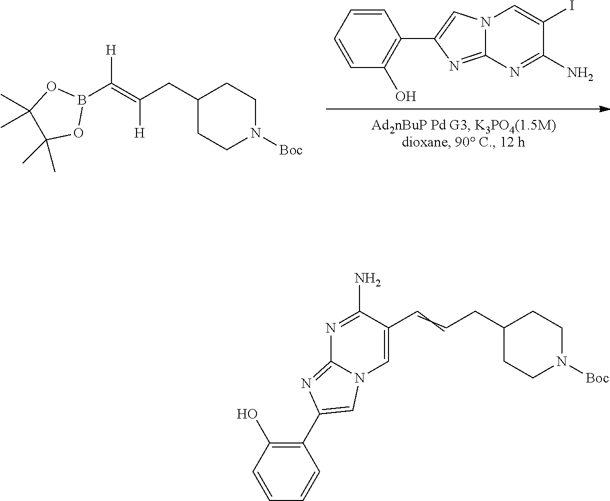 [SMILES: CC(C)(C)OC(=O)N1CCC(C/C=C/B2OC(C)(C)C(C)(C)O2)CC1 ; Nc1nc2nc(-c3ccccc3O)cn2cc1I ; CC(C)(C)OC(=O)N1CCC(CC=Cc2cn3cc(-c4ccccc4O)nc3nc2N)CC1]

A mixture of tert-butyl 4-[3-(4,4,5,5-tetramethyl-1,3,2-dioxaborolan-2-yl)allyl]piperidine-1-carboxylate (599 mg, 1.70 mmol, 2 eq), 2-(7-amino-6-iodo-imidazo[1,2-a]pyrimidin-2-yl) phenol (300 mg, 0.85 mmol, 1 eq), potassium phosphate (1.5 M, 5 mL, 8.80 eq) and (di(1-adamantyl)-butylphosphine)-2-(2′-amino-1,1′-biphenyl)]palladium(II) methanesulfonate, (cataCXium-A-Pd-G3) (62 mg, 0.08 mmol, 0.1 eq) in dioxane (30 mL) was degassed and purged with nitrogen 3 times, and then the mixture was stirred at 90° C. for 12 hours under nitrogen. The reaction was quenched with water (200 mL). The solution was extracted with ethyl acetate (500 mL×2). The organic layers were combined, dried over anhydrous sodium sulfate, filtered, and concentrated under reduced pressure. The residue was purified by prep-HPLC (column: Phenomenex luna C18 150*40 mm*15 um; mobile phase: [water (0.1% TFA)-ACN]; B %: 25%-55%, 10 min). Compound tert-butyl 4-[3-[7-amino-2-(2-hydroxyphenyl)imidazo[1,2-a]pyrimidin-6-yl]allyl]piperidine-1-carboxylate (250 mg, 0.26 mmol) was obtained as a white solid.

### Step 3

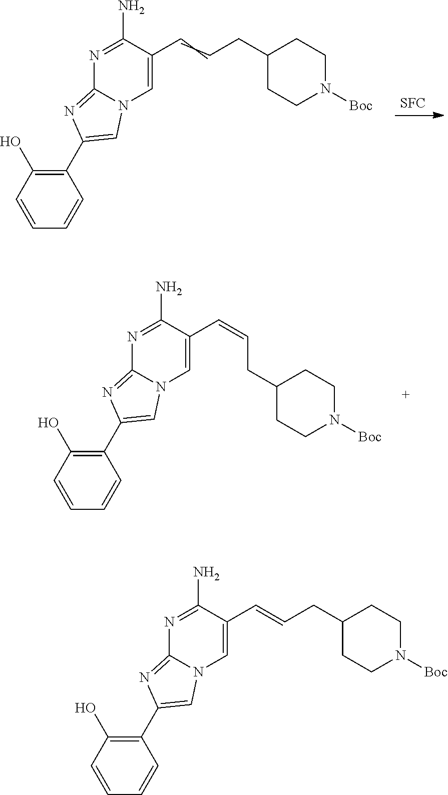

The mixture of E- and Z-isomers of tert-butyl 4-[3-[7-amino-2-(2-hydroxyphenyl)imidazo[1,2-a]pyrimidin-6-yl]allyl]piperidine-1-carboxylate (150 mg, 0.33 mmol, 1 eq) was purified by SFC. Column: DAICEL CHIRALPAK AD 250×30 mm, I.D., 10 um; Mobile phase: isopropanol (0.1% NH3H2O) in CO₂ from 50% to 50%; Flow rate: 70 mL/min; Wavelength: 220 nm). Compound tert-butyl 4-[(Z)-3-[7-amino-2-(2-hydroxyphenyl)imidazo[1,2-a]pyrimidin-6-yl]allyl]piperidine-1-carboxylate (12 mg, 0.03 mmol) was obtained as a white solid. Compound tert-butyl 4-[(E)-3-[7-amino-2-(2-hydroxyphenyl)imidazo[1,2-a]pyrimidin-6-yl]allyl]piperidine-1-carboxylate (130 mg, 0.29 mmol) was obtained as a white solid.

### Step 4

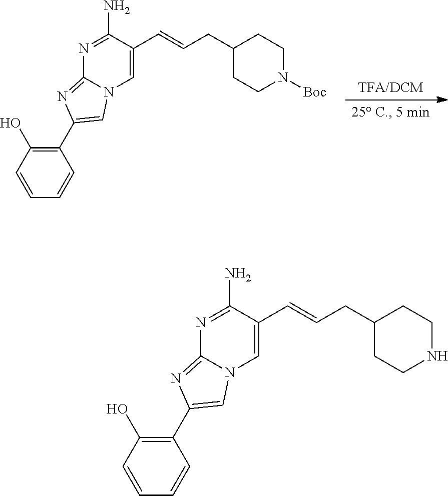 [SMILES: CC(C)(C)OC(=O)N1CCC(C/C=C/c2cn3cc(-c4ccccc4O)nc3nc2N)CC1 ; "25° C., 5 min" ; "TFA/DCM" ; Nc1nc2nc(-c3ccccc3O)cn2cc1/C=C/CC1CCNCC1]

To a solution of tert-butyl 4-[(E)-3-[7-amino-2-(2-hydroxyphenyl)imidazo[1,2-a]pyrimidin-6-yl]allyl]piperidine-1-carboxylate (120 mg, 0.27 mmol, 1 eq) in dichloromethane (5 mL) was added trifluoroacetic acid (1.85 g, 16.21 mmol, 1.20 mL, 60.72 eq). The mixture was stirred at 25° C. for 5 minutes. The reaction mixture was concentrated, water (100 mL) and methanol (50 mL) were added, and sodium bicarbonate was added until pH reached 8. The organic layer was extracted with ethyl acetate (200 mL×3). The combined organic layer was dried over anhydrous sodium sulfate, filtered, and concentrated under vacuum. Crude 2-[7-amino-6-[(E)-3-(4-piperidyl)prop-1-enyl]imidazo[1,2-a]pyrimidin-2-yl]phenol (90 mg, 0.26 mmol) was obtained as a yellow solid and used in the next step without purification.

### Step 5

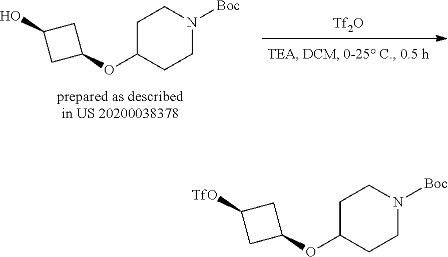 [SMILES: "Tf2O" ; CC(C)(C)OC(=O)N1CCC(O[C@H]2C[C@@H](O)C2)CC1 ; CC(C)(C)OC(=O)N1CCC(O[C@H]2C[C@@H](OS(=O)(=O)C(F)(F)F)C2)CC1]

To a solution of tert-butyl 4-(3-hydroxycyclobutoxy)piperidine-1-carboxylate (4 g, 14.74 mmol, 1 eq) and triethylamine (4.47 g, 44.22 mmol, 6.16 mL, 3 eq) in dichloromethane (120 mL) was added trifluoromethanesulfonic anhydride (4.57 g, 16.22 mmol, 2.68 mL, 1.1 eq) at 0° C. The mixture was stirred at 25° C. for 0.5 hours. The reaction was quenched with water (20 mL). The solution was extracted with dichloromethane (20 mL×2). The organic layers were combined, dried over anhydrous sodium sulfate, filtered, and concentrated under reduced pressure. The residue was purified by silica gel chromatography (2-5% ethyl acetate in petroleum ether). Compound tert-butyl 4-[3-(trifluoromethylsulfonyloxy)cyclobutoxy]piperidine-1-carboxylate (2.5 g, 6.20 mmol, 42% yield) was obtained as a yellow solid.

### Step 6

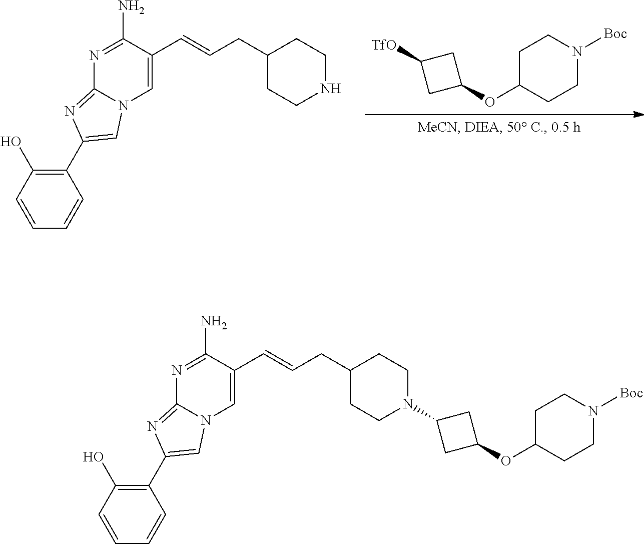

A mixture of 2-[7-amino-6-[(E)-3-(4-piperidyl)prop-1-enyl]imidazo[1,2-a]pyrimidin-2-yl] phenol (90 mg, 0.26 mmol, 1 eq), tert-butyl 4-[3-(trifluoromethylsulfonyloxy)cyclobutoxy] piperidine-1-carboxylate (208 mg, 0.51 mmol, 2 eq), and N,N-diisopropylethylamine (100 mg, 0.77 mmol, 3 eq) in acetonitrile (4 mL) was degassed, purged with nitrogen 3 times, and then stirred at 50° C. for 0.5 hours under nitrogen. The reaction was quenched with water (20 mL). The solution was extracted with ethyl acetate (50 mL×2). The organic layers were combined, dried over anhydrous sodium sulfate, filtered, and concentrated under reduced pressure. The residue was purified by prep-HPLC (column: Phenomenex Synergi C18 150*25 mm*10 um; mobile phase: [water (0.1% TFA)-ACN]; B %: 20%-50%, 40 min). Compound tert-butyl 4-[3-[4-[(E)-3-[7-amino-2-(2-hydroxyphenyl)imidazo[1,2-a]pyrimidin-6-yl]allyl]-1-piperidyl]cyclobutoxy]piperidine-1-carboxylate (130 mg, 0.21 mmol) was obtained as a white solid.

tert-butyl 4-[3-[4-[(E)-3-[7-amino-2-(2-hydroxyphenyl)imidazo[1,2-a]pyrimidin-6-yl]allyl]-1-piperidyl]cyclobutoxy]piperidine-1-carboxylate was converted to the title compound according to the scheme below as described for Exemplary Compound 38 and other Examples above.

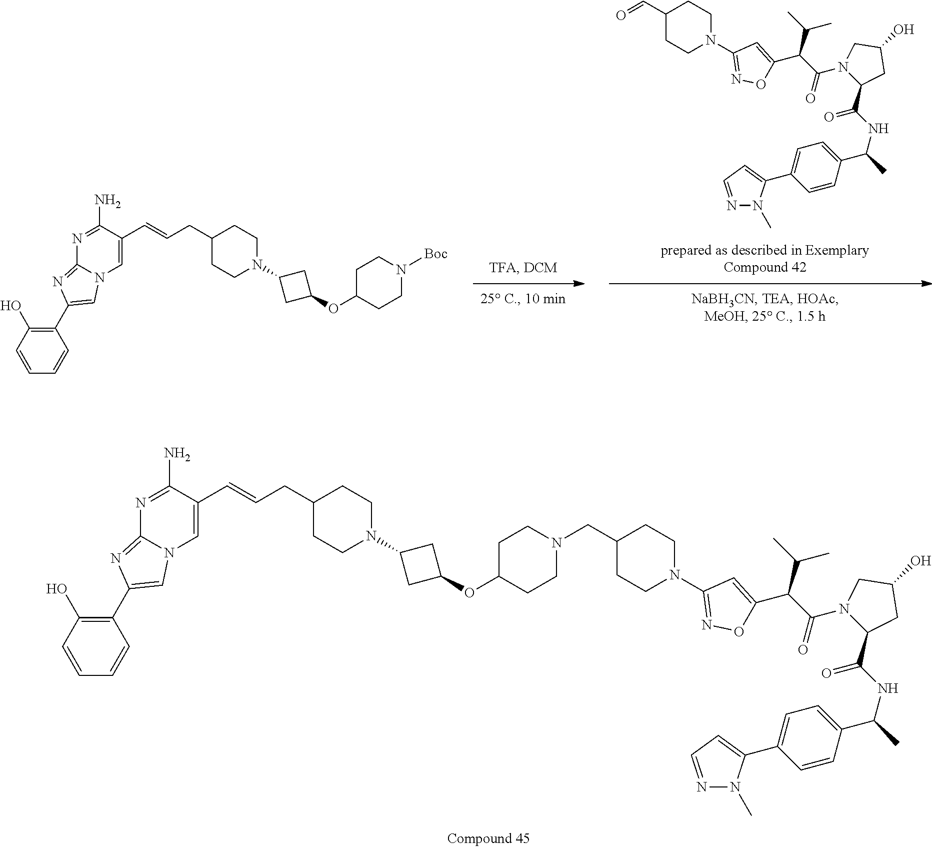 [SMILES: CC(C)(C)OC(=O)N1CCC(O[C@H]2C[C@H](N3CCC(C/C=C/c4cn5cc(-c6ccccc6O)nc5nc4N)CC3)C2)CC1 ; CC(C)[C@@H](C(=O)N1C[C@H](O)C[C@H]1C(=O)N[C@@H](C)c1ccc(-c2ccnn2C)cc1)c1cc(N2CCC(C=O)CC2)no1 ; CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](N5CCC(C/C=C/c6cn7cc(-c8ccccc8O)nc7nc6N)CC5)C4)CC3)CC2)no1]

### Exemplary Synthesis of Compound 46

### Step 1

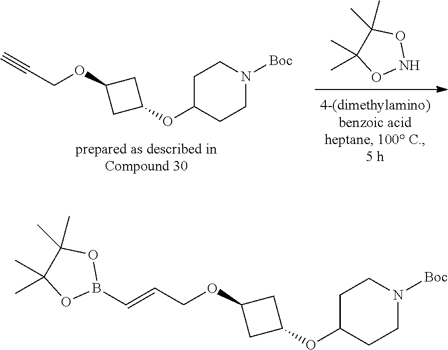

To a solution of tert-butyl 4-(3-prop-2-ynoxycyclobutoxy)piperidine-1-carboxylate (0.9 g, 2.91 mmol, 1 eq) in heptane (40 mL) was added 4-(dimethylamino)benzoic acid (48 mg, 0.29 mmol, 0.1 eq) and pinacolborane (1.12 g, 8.73 mmol, 1.3 mL, 3 eq). The mixture was stirred at 100° C. for 5 hours. The reaction was quenched with water (200 mL). The mixture was extracted with ethyl acetate (500 mL×2). The organic layers were combined, dried over anhydrous sodium sulfate, filtered and concentrated under reduced pressure. The residue was purified by prep-HPLC (column: Welch Ultimate XB-CN 250*50*10 um; mobile phase: [Hexane-EtOH (0.1% NH3·H2O)]; B %: 1%-30%, 15 min). Compound tert-butyl 4-[3-[(E)-3-(4,4,5,5-tetramethyl-1,3,2-dioxaborolan-2-yl)allyloxy]cyclobutoxy]piperidine-1-carboxylate (800 mg, 1.83 mmol) was obtained as a colorless oil.

tert-Butyl 4-[3-[(E)-3-(4,4,5,5-tetramethyl-1,3,2-dioxaborolan-2-yl)allyloxy]cyclobutoxy]piperidine-1-carboxylate was converted to the title compound according to the scheme below as described for Exemplary Compound 44.

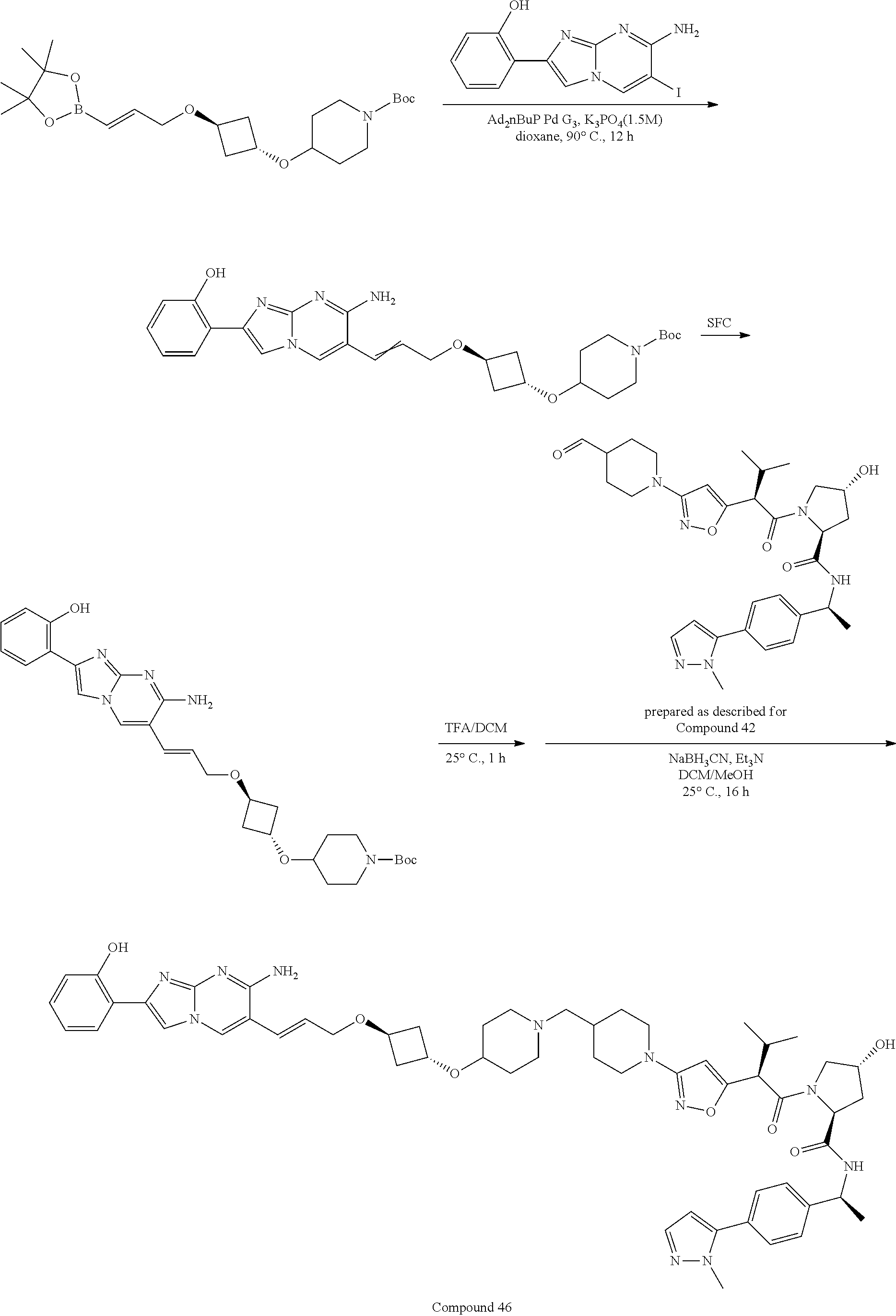

### Exemplary Synthesis of Compound 47

### Step 1

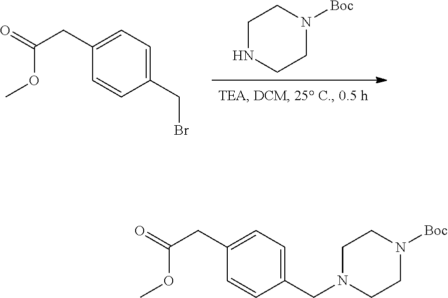 [SMILES: COC(=O)Cc1ccc(CBr)cc1 ; CC(C)(C)OC(=O)N1CCNCC1 ; COC(=O)Cc1ccc(CN2CCN(C(=O)OC(C)(C)C)CC2)cc1]

To a solution of methyl 2-[4-(bromomethyl)phenyl]acetate (3 g, 12.34 mmol, 1 eq) and tert-butyl piperazine-1-carboxylate (4.60 g, 24.68 mmol, 2 eq) in dichloromethane (30 mL) was added triethylamine (3.75 g, 37.02 mmol, 5.2 mL, 3 eq). The mixture was stirred at 25° C. for 0.5 hours. 50 mL water was added to the mixture, and the mixture was extracted with ethyl acetate (100 mL×3). The combined organic phase was washed with brine (50 mL×2), dried with anhydrous sodium sulfate, filtered, and concentrated in vacuum. The residue was purified by silica gel chromatography (petroleum ether/ethyl acetate=100/1 to 10/1). tert-Butyl 4-[[4-(2-methoxy-2-oxo-ethyl)phenyl]methyl]piperazine-1-carboxylate (3.8 g, 10.91 mmol, 88% yield) was obtained as a colorless oil.

### Step 2

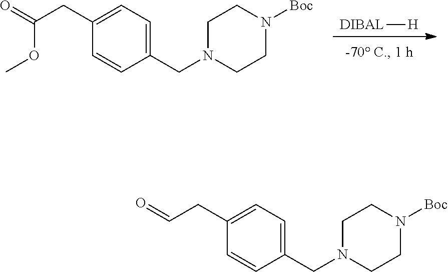

To a solution of tert-butyl 4-[[4-(2-methoxy-2-oxo-ethyl)phenyl]methyl]piperazine-1-carboxylate (3.8 g, 10.91 mmol, 1 eq) in dichloromethane (40 mL) was added diisobutyl aluminium hydride (1 M, 22 mL, 2 eq). The mixture was stirred at −70° C. for 1 hour under nitrogen. 40 mL of methanol was added slowly at 0° C. under nitrogen, and the mixture was stirred for 15 minutes. 100 mL of water was added, and the mixture was extracted with ethyl acetate (100 mL×3). The combined organic phase was washed with brine (30 mL×2), dried with anhydrous sodium sulfate, filtered, and concentrated in vacuum. Crude tert-butyl 4-[[4-(2-oxoethyl)phenyl]methyl]piperazine-1-carboxylate (3.4 g, 10.68 mmol) was obtained as a colorless oil and used in the next step without purification.

### Step 3

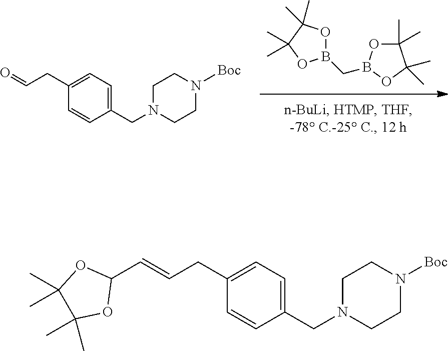

To a solution of 2,2,6,6-tetramethylpiperidine (1.81 g, 12.81 mmol, 2.2 mL, 1.2 eq) in tetrahydrofuran (20 mL) was added dropwise n-butyllithium (2.5 M, 5.2 mL, 1.2 eq) at 0° C., and then the mixture was stirred for 0.5 hours at 0° C. under nitrogen. After that a solution of 4,4,5,5-tetramethyl-2-[(4,4,5,5-tetramethyl-1,3,2-dioxaborolan-2-yl)methyl]-1,3,2-dioxaborolane (3.43 g, 12.81 mmol, 1.2 eq) in tetrahydrofuran (10 mL) was added dropwise at −78° C., followed by the addition of a solution of tert-butyl 4-[[4-(2-oxoethyl)phenyl]methyl]piperazine-1-carboxylate (3.4 g, 10.68 mmol, 1 eq) in tetrahydrofuran (10 mL) dropwise at −78° C. The mixture was slowly warmed up to 25° C. and stirred for 12 hours at 25° C. 50 mL of water was added to the mixture, and the mixture was extracted with ethyl acetate (100 mL×3). The combined organic phase was washed with brine (50 mL×2), dried with anhydrous sodium sulfate, filtered, and concentrated in vacuum. The residue was purified by prep-HPLC (column: Welch Ultimate XB-CN 250*70*10 um; mobile phase: [Hexane-EtOH (0.1% NH3·H2O)]; B %: 1%-30%, 15 min). tert-Butyl 4-[[4-[(E)-3-(4,4,5,5-tetramethyl-1,3,2-dioxaborolan-2-yl)allyl]phenyl]methyl]piperazine-1-carboxylate (1.2 g, 2.71 mmol) was obtained as a colorless oil.

tert-Butyl 4-[[4-[(E)-3-(4,4,5,5-tetramethyl-1,3,2-dioxaborolan-2-yl)allyl]phenyl]methyl]piperazine-1-carboxylate was converted to (E)-2-(7-amino-6-(3-(4-(piperazin-1-ylmethyl)phenyl)prop-1-en-1-yl)imidazo[1,2-a]pyrimidin-2-yl)phenol as shown in the scheme below using procedures described in steps 2-4 for exemplary compound 45.

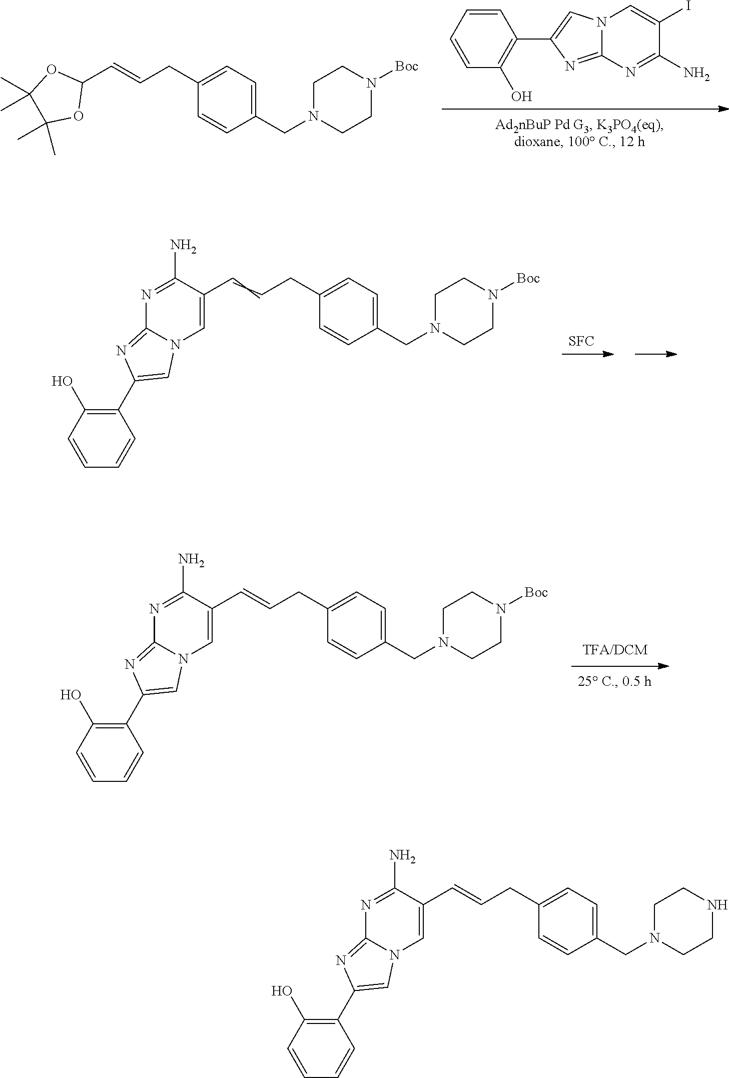 [SMILES: CC(C)(C)OC(=O)N1CCN(Cc2ccc(C/C=C/C3OC(C)(C)C(C)(C)O3)cc2)CC1 ; Nc1nc2nc(-c3ccccc3O)cn2cc1I ; CC(C)(C)OC(=O)N1CCN(Cc2ccc(CC=Cc3cn4cc(-c5ccccc5O)nc4nc3N)cc2)CC1 ; "SFC" ; CC(C)(C)OC(=O)N1CCN(Cc2ccc(C/C=C/c3cn4cc(-c5ccccc5O)nc4nc3N)cc2)CC1 ; Nc1nc2nc(-c3ccccc3O)cn2cc1/C=C/Cc1ccc(CN2CCNCC2)cc1]

### Step 4

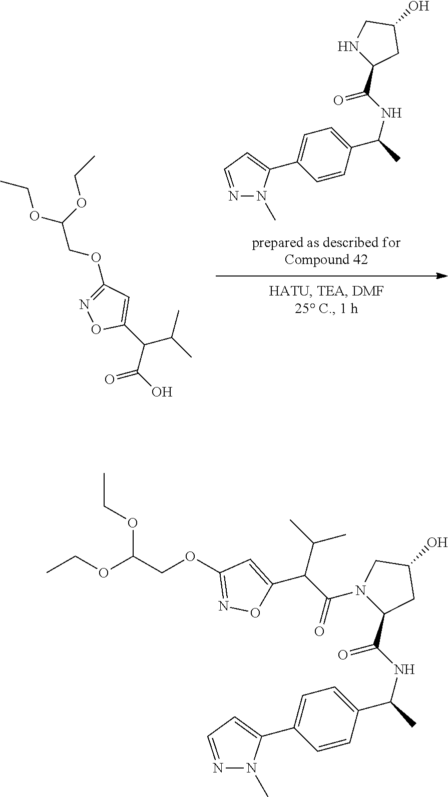 [SMILES: CCOC(COc1cc(C(C(=O)O)C(C)C)on1)OCC ; C[C@H](NC(=O)[C@@H]1C[C@@H](O)CN1)c1ccc(-c2ccnn2C)cc1 ; CCOC(COc1cc(C(C(=O)N2C[C@H](O)C[C@H]2C(=O)N[C@@H](C)c2ccc(-c3ccnn3C)cc2)C(C)C)on1)OCC]

To a solution of 2-[3-(2,2-diethoxyethoxy)isoxazol-5-yl]-3-methyl-butanoic acid (429 mg, 1.43 mmol, 1 eq) and (2S,4R)-4-hydroxy-N-[(1S)-1-[4-(2-methylpyrazol-3-yl)phenyl]ethyl] pyrrolidine-2-carboxamide hydrochloride (500 mg, 1.43 mmol, 1 eq) in N,N-dimethylformamide (5 mL) was added triethylamine (432 mg, 4.28 mmol, 0.6 mL, 3 eq) and O-(7-azabenzotriazol-1-yl)-N,N,N′,N′-tetramethyluronium hexafluorophosphate (812 mg, 2.14 mmol, 1.5 eq). The mixture was stirred at 25° C. for 1 hour. 50 mL of water was added to the mixture, and then the mixture was extracted with ethyl acetate (50 mL×3). The combined organic phase was washed with brine (30 mL×2), dried with anhydrous sodium sulfate, filtered, and concentrated in vacuum. The residue was purified by prep-HPLC (column: Phenomenex luna C18 (250*70 mm, 15 um); mobile phase: [water (0.225% FA)-ACN]; B %: 37ACN %-67ACN %, 18 min). (2S,4R)-1-[2-[3-(2,2-Diethoxyethoxy)isoxazol-5-yl]-3-methyl-butanoyl]-4-hydroxy-N-[(1S)-1-[4-(2-methylpyrazol-3-yl)phenyl]ethyl]pyrrolidine-2-carboxamide (600 mg, 1.00 mmol) was obtained as a white solid.

### Step 5

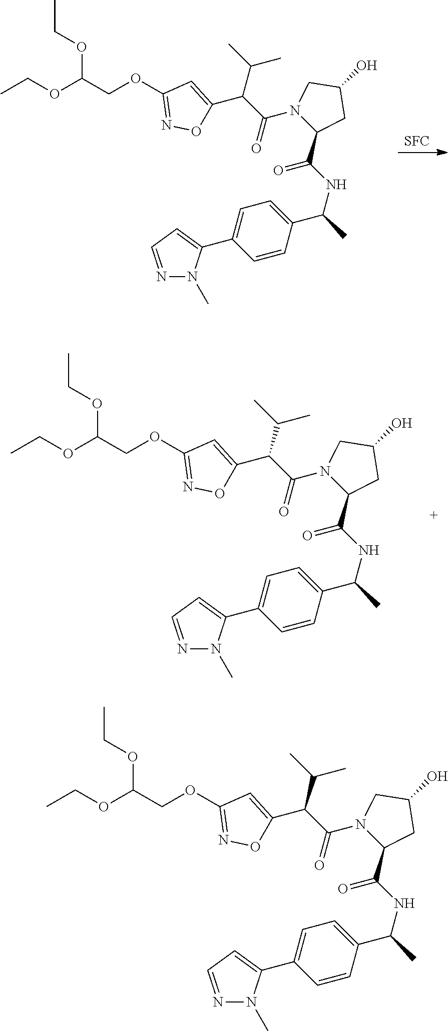

The compound (2S,4R)-1-[2-[3-(2,2-diethoxyethoxy)isoxazol-5-yl]-3-methyl-butanoyl]-4-hydroxy-N-[(1S)-1-[4-(2-methylpyrazol-3-yl)phenyl]ethyl]pyrrolidine-2-carboxamide (600 mg, 1.00 mmol) was separated into individual diastereomers by chiral SFC (Column: DAICEL CHIRALPAK AD 250×30 mm, I.D., 10 um; Mobile phase: isopropanol (0.1% NH3H2O) in CO₂ from 35% to 35%; Flow rate: 65 mL/min; Wavelength: 220 nm). (2S,4R)-1-[(2S)-2-[3-(2,2-Diethoxyethoxy)isoxazol-5-yl]-3-methyl-butanoyl]-4-hydroxy-N-[(1S)-1-[4-(2-methylpyrazol-3-yl)phenyl]ethyl]pyrrolidine-2-carboxamide (160 mg, 0.27 mmol) and (2S,4R)-1-[(2R)-2-[3-(2,2-diethoxy ethoxy)isoxazol-5-yl]-3-methyl-butanoyl]-4-hydroxy-N-[(1S)-1-[4-(2-methylpyrazol-3-yl)phenyl]ethyl]pyrrolidine-2-carboxamide (300 mg, 0.5 mmol) were obtained as white solids.

### Step 6

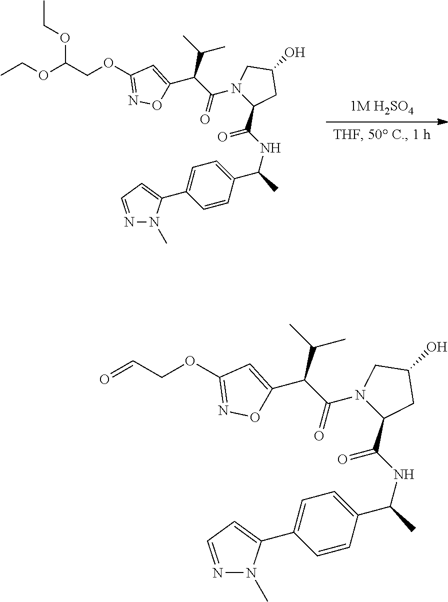 [SMILES: CCOC(COc1cc([C@H](C(=O)N2C[C@H](O)C[C@H]2C(=O)N[C@@H](C)c2ccc(-c3ccnn3C)cc2)C(C)C)on1)OCC ; "1M H2SO4" ; "THF, 50° C., 1 h" ; CC(C)[C@@H](C(=O)N1C[C@H](O)C[C@H]1C(=O)N[C@@H](C)c1ccc(-c2ccnn2C)cc1)c1cc(OCC=O)no1]

To a solution of (2S,4R)-1-[(2R)-2-[3-(2,2-diethoxyethoxy)isoxazol-5-yl]-3-methyl-butanoyl]—To a solution of 4-hydroxy-N-[(1S)-1-[4-(2-methylpyrazol-3-yl)phenyl]ethyl]pyrrolidine-2-carboxamide (100 mg, 0.17 mmol, 1 eq) in tetrahydrofuran (1 mL) was added sulfuric acid (1 M, 1 mL, 4.60 eq). The mixture was stirred at 50° C. for 1 hour. The reaction mixture was quenched by saturated sodium bicarbonate solution, and the aqueous phase was extracted with ethyl acetate (50 mL×3). The combined organic phase was washed with brine (50 mL×2), dried with anhydrous sodium sulfate, filtered, and concentrated in vacuum. (2S,4R)-4-hydroxy-1-[(2R)-3-methyl-2-[3-(2-oxoethoxy)isoxazol-5-yl] butanoyl]-N-[(1S)-1-[4-(2-methylpyrazol-3-yl)phenyl]ethyl]pyrrolidine-2-carboxamide (80 mg, 0.15 mmol) was obtained as a white solid and used in the next step without purification.

### Step 7

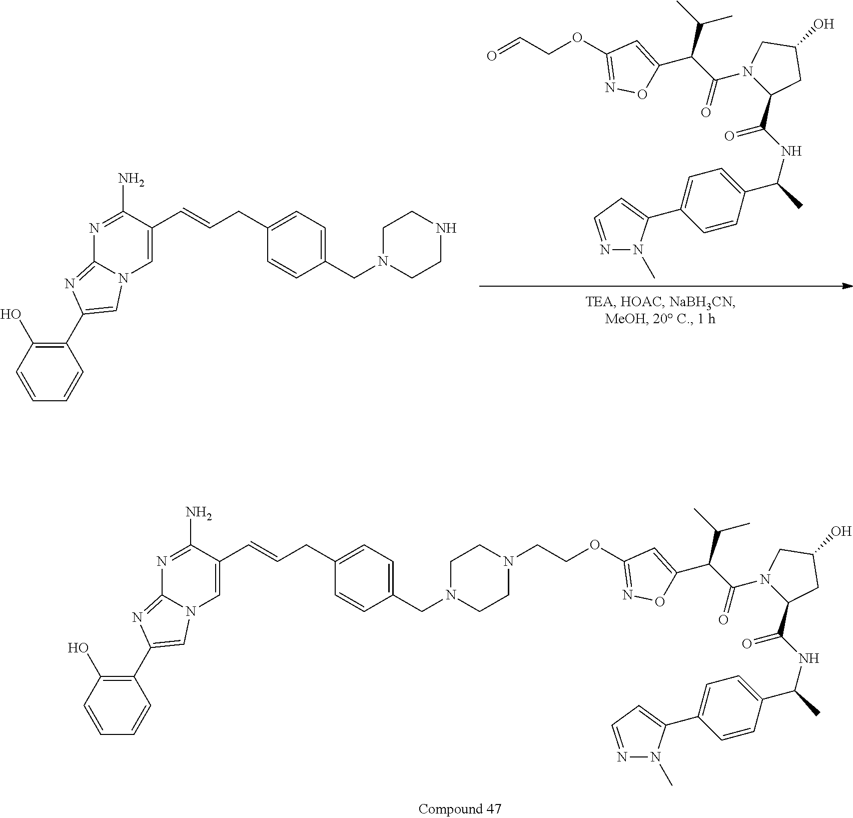 [SMILES: Nc1nc2nc(-c3ccccc3O)cn2cc1/C=C/Cc1ccc(CN2CCNCC2)cc1 ; CC(C)[C@@H](C(=O)N1C[C@H](O)C[C@H]1C(=O)N[C@@H](C)c1ccc(-c2ccnn2C)cc1)c1cc(OCC=O)no1 ; CC(C)[C@@H](C(=O)N1C[C@H](O)C[C@H]1C(=O)N[C@@H](C)c1ccc(-c2ccnn2C)cc1)c1cc(OCCN2CCN(Cc3ccc(C/C=C/c4cn5cc(-c6ccccc6O)nc5nc4N)cc3)CC2)no1]

To a solution of 2-[7-amino-6-[(E)-3-[4-(piperazin-1-ylmethyl)phenyl]prop-1-enyl]imidazo [1,2-a]pyridin-2-yl]phenol trifluoroacetate (63 mg, 0.11 mmol, 1 eq) in methanol (1 mL) was added triethylamine (35 mg, 0.34 mmol, 0.1 mL, 3 eq), followed by (2S,4R)-4-hydroxy-1-[(2R)-3-methyl-2-[3-(2-oxoethoxy)isoxazol-5-yl]butanoyl]-N-[(1S)-1-[4-(2-methylpyrazol-3-yl)phenyl]ethyl]pyrrolidine-2-carboxamide (60 mg, 0.11 mmol, 1 eq) and acetic acid (14 mg, 0.23 mmol, 2 eq), and the mixture was stirred at 25° C. for 0.5 hours. To the mixture was then added sodium cyanoborohydride (28 mg, 0.45 mmol, 4 eq), and the solution was stirred at 25° C. for 0.5 hours. The reaction mixture was concentrated under reduced pressure. The residue was purified by prep-HPLC (column: Phenomenex luna C18 150*25 mm*10 um; mobile phase: [water (0.225% FA)-ACN]; B %: 18%-48%, 10 min). (2S,4R)-1-[(2R)-2-[3-[2-[4-[[4-[(E)-3-[7-Amino-2-(2-hydroxyphenyl) imidazo[1,2-a]pyrimidin-6-yl]allyl]phenyl]methyl]piperazin-1-yl]ethoxy]isoxazol-5-yl]-3-methyl-butanoyl]-4-hydroxy-N-[(1S)-1-[4-(2-methylpyrazol-3-yl)phenyl]ethyl]pyrrolidine-2-carboxamide (62.4 mg, 0.06 mmol) was obtained as a white solid.

### Exemplary Synthesis of Compound 1

### Step 1

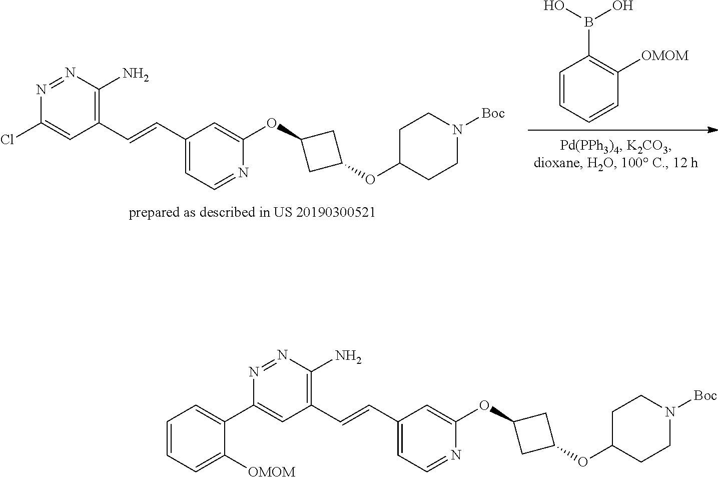

To a solution of tert-butyl 4-[3-[[4-[(E)-2-(3-amino-6-chloro-pyridazin-4-yl)vinyl]-2-pyridyl] oxy]cyclobutoxy]piperidine-1-carboxylate (1.1 g, 2.19 mmol, 1 eq) and [2-(methoxymethoxy) phenyl]boronic acid (598 mg, 3.29 mmol, 1.5 eq) in dioxane (10 mL) and water (2 mL) was added tetrakis(triphenylphosphine)palladium(0) (253 mg, 0.2 mmol, 0.1 eq) and potassium carbonate (605 mg, 4.38 mmol, 2 eq) under nitrogen. The mixture was stirred at 100° C. for 12 hours under nitrogen. Water (50 mL) was added to the mixture. The aqueous phase was extracted with ethyl acetate (50 mL×3). The combined organic phase was washed with brine (50 mL), dried over sodium sulfate, filtered and concentrated in vacuum. The residue was purified by silica gel chromatography (petroleum ether: ethyl acetate=100:1 to 20:1). tert-Butyl 4-[3-[[4-[(E)-2-[3-amino-6-[2-(methoxymethoxy)phenyl]pyridazin-4-yl]vinyl]-2-pyridyl]oxy]cyclobutoxy]piperidine-1-carboxylate (1 g, 1.37 mmol) was obtained as a yellow gum.

### Step 2

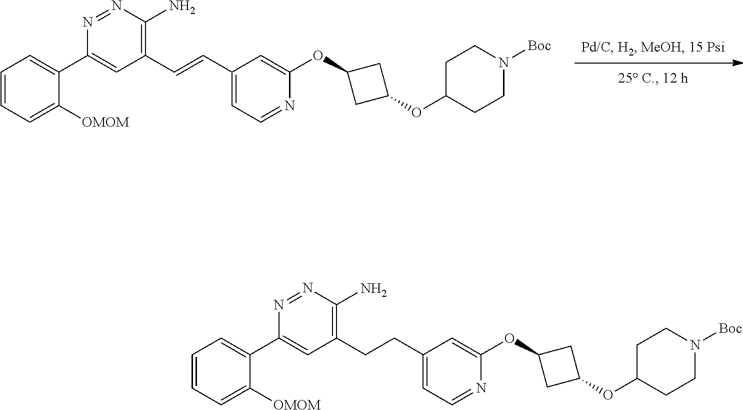

To a solution of tert-butyl 4-[3-[[4-[(E)-2-[3-amino-6-[2-(methoxymethoxy)phenyl]pyridazin-4-yl]vinyl]-2-pyridyl]oxy]cyclobutoxy]piperidine-1-carboxylate (80 mg, 0.13 mmol, 1 eq) in methanol (10 mL) was added 10% palladium on activated carbon catalyst (30 mg). The mixture was stirred at 25° C. for 12 hours under hydrogen pressure (15 psi). The reaction mixture was filtered and concentrated under reduced pressure. Compound tert-butyl 4-[3-[[4-[2-[3-amino-6-[2-(methoxymethoxy)phenyl]pyridazin-4-yl]ethyl]-2-pyridyl]oxy]cyclobutoxy]piperidine-1-carboxylate (80 mg, 0.13 mmol) was obtained as a yellow solid.

tert-Butyl 4-[3-[[4-[2-[3-amino-6-[2-(methoxymethoxy)phenyl]pyridazin-4-yl]ethyl]-2-pyridyl]oxy]cyclobutoxy]piperidine-1-carboxylate was converted to the title compound according to the scheme below using procedures described above.

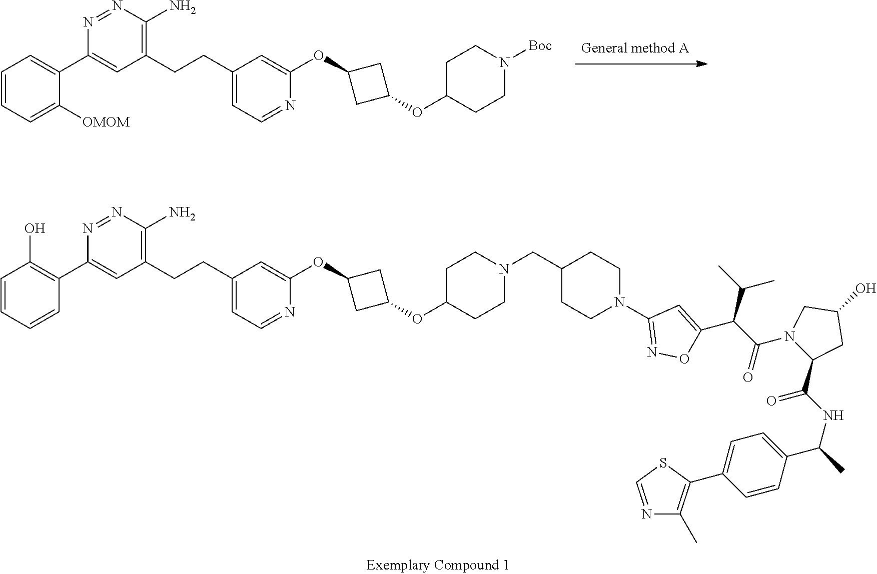

### Exemplary Synthesis of Compound 2

### Step 1

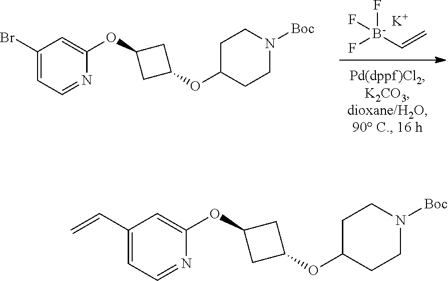

prepared as described in US 20190300521

To a mixture of tert-butyl 4-[3-[(4-bromo-2-pyridyl)oxy]cyclobutoxy]piperidine-1-carboxylate (5 g, 11.70 mmol, 1 eq), potassium carbonate (4.85 g, 35.10 mmol, 3 eq) and potassium trifluoro(vinyl)borate (3.13 g, 23.40 mmol, 2 eq) in dioxane (112 mL) and water (18.5 mL) was added [1,1′-bis(diphenylphosphino)ferrocene]dichloropalladium(II) (856 mg, 1.17 mmol, 0.1 eq) in one portion at 25° C. under nitrogen. The mixture was stirred at 90° C. for 16 hours. The mixture was poured into ice-water (1/1, 20 mL) and stirred for 5 min. The aqueous phase was extracted with ethyl acetate (50 mL×2). The combined organic phase was washed with brine (30 mL), dried with anhydrous sodium sulfate, filtered and concentrated in vacuum. The residue was purified by silica gel chromatography (petroleum ether/ethyl acetate=10/1, 5/1). Compound tert-butyl 4-[3-[(4-vinyl-2-pyridyl)oxy]cyclobutoxy]piperidine-1-carboxylate (3 g, 7.95 mmol) was obtained as a yellow oil.

### Step 2

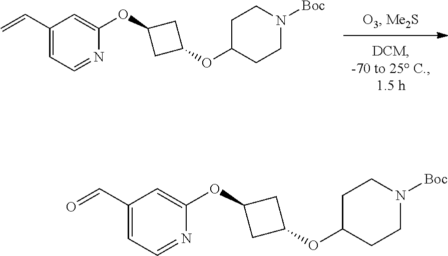 [SMILES: C=Cc1ccnc(O[C@H]2C[C@H](OC3CCN(C(=O)OC(C)(C)C)CC3)C2)c1 ; CC(C)(C)OC(=O)N1CCC(O[C@H]2C[C@H](Oc3cc(C=O)ccn3)C2)CC1]

Ozone was bubbled into a solution of tert-butyl 4-[3-[(4-vinyl-2-pyridyl)oxy]cyclobutoxy] piperidine-1-carboxylate (3 g, 8.01 mmol, 1 eq) in dichloromethane (100 mL) at −70° C. for 30 minutes. After excess ozone was purged by nitrogen, dimethyl sulfide (2.49 g, 40.06 mmol, 2.9 mL, 5 eq) was added at −70° C., and the mixture was warmed to 25° C. and stirred for 1 hours. The reaction mixture was concentrated. The residue was purified by silica gel chromatography (petroleum ether/ethyl acetate from 15/1 to 3/1) to produce tert-butyl 4-[3-[(4-formyl-2-pyridyl)oxy]cyclobutoxy] piperidine-1-carboxylate (1.32 g, 3.51 mmol) as a yellow solid.

### Step 3

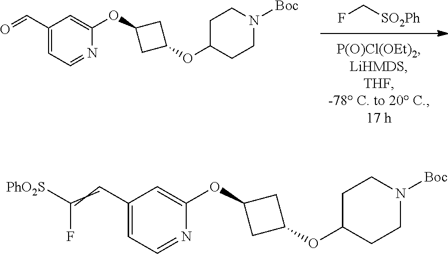

Hexane solution of lithiumbis(trimethylsilyl)amide (1 M, 3.0 mL, 2 eq) and diethyl chlorophosphate (385 mg, 2.23 mmol, 0.3 mL, 1.5 eq) were added sequentially to a solution of fluoromethylsulfonylbenzene (388 mg, 2.23 mmol, 1.5 eq) in tetrahydrofuran (3 mL), and the reaction was stirred at −78° C. for 1 hour. tert-Butyl 4-[3-[(4-formyl-2-pyridyl)oxy] cyclobutoxy]piperidine-1-carboxylate (0.56 g, 1.49 mmol, 1 eq) was then added at −78° C. under nitrogen. The resulting mixture was warmed to 20° C. and stirred for 16 hours. The mixture was poured into a saturated aqueous solution of ammonium chloride (50 mL) and stirred for 5 minutes. The aqueous phase was extracted with ethyl acetate (35 mL×3). The combined organic phase was washed with brine (25 mL×2), dried with anhydrous sodium sulfate, filtered and concentrated in vacuum. The residue was purified by semi-preparative reverse phase HPLC (column: Phenomenex Gemini-NX C18 75*30 mm*3 um; mobile phase: [water (0.1% TFA)-ACN]; B %: 70%-80%). Compound tert-butyl 4-((1r,3r)-3-((4-(2-fluoro-2-(phenylsulfonyl)vinyl)pyridin-2-yl)oxy)cyclobutoxy)piperidine-1-carboxylate (0.49 g, 0.92 mmol) was obtained as a yellow solid.

### Step 4

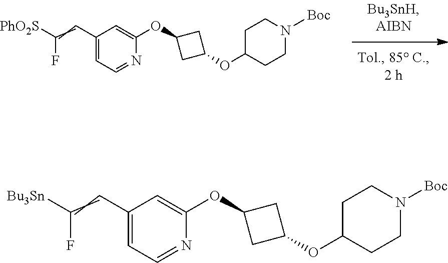

To a solution of tert-butyl 4-((1r,3r)-3-((4-(2-fluoro-2-(phenylsulfonyl)vinyl)pyridin-2-yl)oxy)cyclobutoxy)piperidine-1-carboxylate (390 mg, 0.73 mmol, 1 eq) in toluene (5.1 mL) was added tributyltin hydride (213 mg, 0.73 mmol, 0.2 mL, 1 eq) and 2,2-azobisisobutyronitrile (30 mg, 0.18 mmol, 0.25 eq) in one portion at 20° C. under nitrogen. The mixture was heated to 85° C. and stirred for 2 hours. The mixture was cooled to 20° C. and concentrated under reduced pressure at 45° C. The residue was purified by preparative TLC (petroleum ether/ethyl acetate=3/1) to afford tert-butyl 4-((1r,3r)-3-((4-(2-fluoro-2-(tributylstannyl)vinyl)pyridin-2-yl)oxy)cyclobutoxy)piperidine-1-carboxylate (400 mg, 0.59 mmol) as a yellow solid.

### Step 5

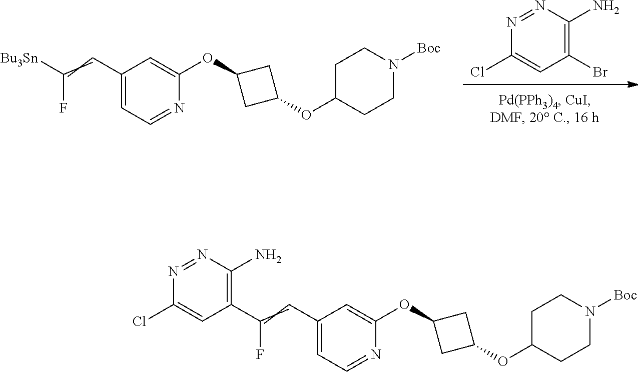

To a mixture of tert-butyl 4-((1r,3r)-3-((4-(2-fluoro-2-(tributylstannyl)vinyl)pyridin-2-yl)oxy)cyclobutoxy)piperidine-1-carboxylate (400 mg, 0.58 mmol, 1 eq) and 4-bromo-6-chloro-pyridazin-3-amine (159 mg, 0.76 mmol, 1.3 eq) in N,N-dimethylformamide (10 mL) was added tetrakis(triphenylphosphine)palladium (0) (68 mg, 0.06 mmol, 0.1 eq) and cuprous iodide (56 mg, 0.29 mmol, 0.5 eq) in one portion at 20° C. under nitrogen. The mixture was stirred at 20° C. for 16 hours. The mixture was concentrated under reduced pressure at 45° C. The residue was purified by semi-preparative reverse phase HPLC (column: Phenomenex Gemini-NX C18 75*30 mm*3 um; mobile phase: [water (0.1% TFA)-ACN]; B %: 58%-68%). Compound tert-butyl 4-((1r,3r)-3-((4-(2-(3-amino-6-chloropyridazin-4-yl)-2-fluorovinyl)pyridin-2-yl)oxy)cyclobutoxy)piperidine-1-carboxylate (268 mg, 0.51 mmol) was obtained as a yellow solid.

### Step 6

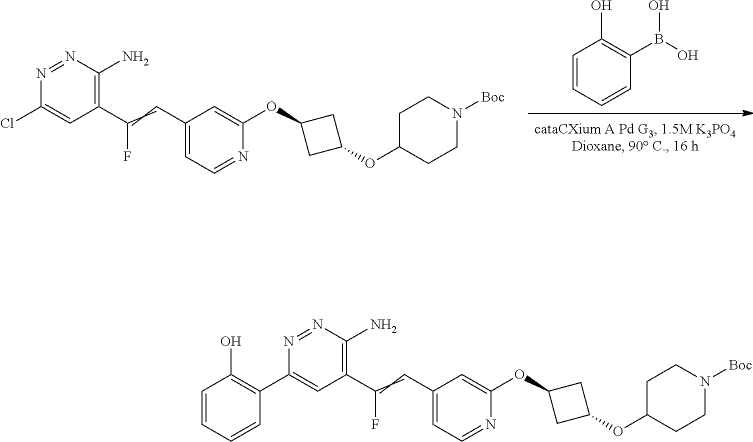 [SMILES: CC(C)(C)OC(=O)N1CCC(O[C@H]2C[C@H](Oc3cc(C=C(F)c4cc(Cl)nnc4N)ccn3)C2)CC1 ; OB(O)c1ccccc1O ; CC(C)(C)OC(=O)N1CCC(O[C@H]2C[C@H](Oc3cc(C=C(F)c4cc(-c5ccccc5O)nnc4N)ccn3)C2)CC1]

To a mixture of (2-hydroxyphenyl)boronic acid (106 mg, 0.77 mmol, 1.5 eq), potassium phosphate (1.5 M, 1.0 mL, 3 eq) and tert-butyl 4-((1r,3r)-3-((4-(2-(3-amino-6-chloropyridazin-4-yl)-2-fluorovinyl)pyridin-2-yl)oxy)cyclobutoxy)piperidine-1-carboxylate (268 mg, 0.51 mmol, 1 eq) in dioxane (6.3 mL) was added [(di(1-adamantyl)-butylphospine)-2-(2′-amino-1,1′-biphenyl)]palladium(II) methanesulfonate [cataCXium A Pd G3 catalyst](37 mg, 0.05 mmol, 0.1 eq) in one portion at 13° C. under nitrogen. The mixture was stirred at 90° C. for 16 hours. The mixture was cooled to 20° C., poured into ice-water (w/w=1/1, 60 mL) and stirred for 5 minutes. The aqueous phase was extracted with ethyl acetate (50 mL×4). The combined organic phase was washed with brine (15 mL×2), dried with anhydrous sodium sulfate, filtered and concentrated in vacuum. The residue was purified by semi-preparative reverse phase HPLC (column: Phenomenex Gemini-NX C18 75*30 mm*3 um; mobile phase: [water (0.1% TFA)-ACN]; B %: 55%-65%). Compound tert-butyl 4-((1r,3r)-3-((4-(2-(3-amino-6-(2-hydroxyphenyl)pyridazin-4-yl)-2-fluorovinyl)pyridin-2-yl)oxy)cyclobutoxy)piperidine-1-carboxylate (115 mg, 0.20 mmol, 39% yield) was obtained as a yellow solid.

### Step 7

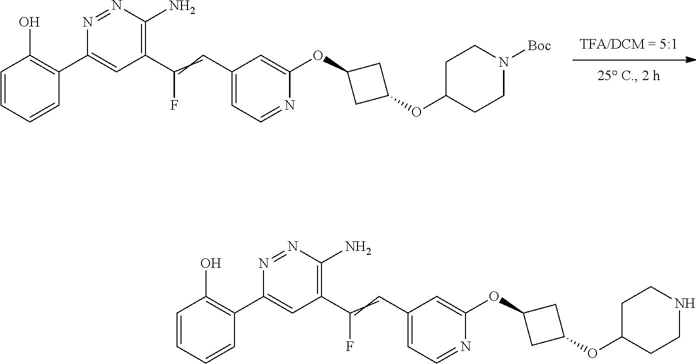 [SMILES: CC(C)(C)OC(=O)N1CCC(O[C@H]2C[C@H](Oc3cc(C=C(F)c4cc(-c5ccccc5O)nnc4N)ccn3)C2)CC1 ; Nc1nnc(-c2ccccc2O)cc1C(F)=Cc1ccnc(O[C@H]2C[C@H](OC3CCNCC3)C2)c1]

A mixture of tert-butyl 4-((1r,3r)-3-((4-(2-(3-amino-6-(2-hydroxyphenyl)pyridazin-4-yl)-2-fluorovinyl)pyridin-2-yl)oxy)cyclobutoxy)piperidine-1-carboxylate (115 mg, 0.20 mmol, 1 eq) and trifluoroacetic acid (1.54 g, 13.51 mmol, 1 mL, 68 eq) in dichloromethane (6 mL) was stirred at 20° C. for 2 hours. The mixture was concentrated in reduced pressure at 40° C. The crude product 2-(6-amino-5-(1-fluoro-2-(2-((1r,3r)-3-(piperidin-4-yloxy)cyclobutoxy)pyridin-4-yl)vinyl)pyridazin-3-yl)phenol trifluoroacetate (117 mg) was obtained as a yellow solid.

### Step 8

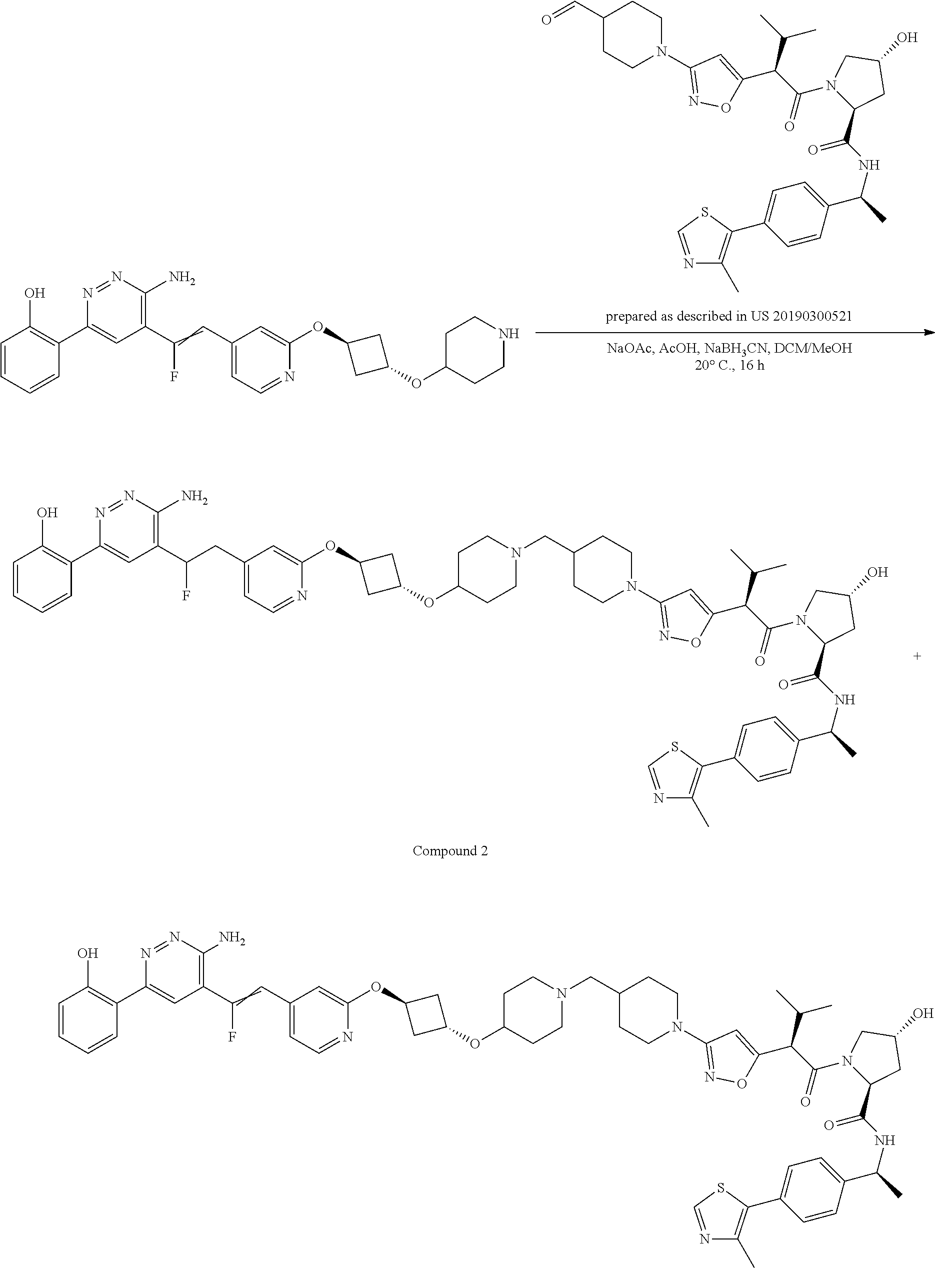 [SMILES: Nc1nnc(-c2ccccc2O)cc1C(F)=Cc1ccnc(O[C@H]2C[C@H](OC3CCNCC3)C2)c1 ; Cc1ncsc1-c1ccc([C@H](C)NC(=O)[C@@H]2C[C@@H](O)CN2C(=O)[C@@H](c2cc(N3CCC(C=O)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Oc6cc(CC(F)c7cc(-c8ccccc8O)nnc7N)ccn6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Oc6cc(C=C(F)c7cc(-c8ccccc8O)nnc7N)ccn6)C5)CC4)CC3)no2)C(C)C)cc1]

To a mixture of 2-[6-amino-5-[1-fluoro-2-[2-[3-[[1-(4-piperidylmethyl)-4-piperidyl]oxy] cyclobutoxy]-4-pyridyl]vinyl] pyridazin-3-yl]phenol trifluoroacetate (117 mg, 0.17 mmol, 1 eq) and (2S,4R)-1-[(2R)-2-[3-(4-formyl-1-piperidyl)isoxazol-5-yl]-3-methyl-butanoyl]-4-hydroxy-N-[(1 S)-1-[4-(4-methylthiazol-5-yl)phenyl] ethyl] pyrrolidine-2-carboxamide (100 mg, 0.17 mmol, 1 eq) in methanol (6 mL) and dichloromethane (2 mL) was added sodium acetate (28 mg, 0.34 mmol, 2 eq), acetic acid (0.17 mmol, 1 eq), and sodium cyanoborohydride (53 mg, 0.85 mmol, 5 eq) in one portion at 20° C. under nitrogen. The mixture was stirred at 20° C. for 16 hours. The mixture was concentrated under reduced pressure at 40° C. The residue was purified by semi-preparative reverse phase HPLC (FA condition; column: Phenomenex luna C18 150*25 mm*10 um; mobile phase: [water (0.225% FA)-ACN]; B %: 26%-56%, 10 min), the crude (2S,4R)-1-((R)-2-(3-(4-((4-((1r,3r)-3-((4-(2-(3-amino-6-(2-hydroxyphenyl)pyridazin-4-yl)-2-fluorovinyl)pyridin-2-yl)oxy)cyclobutoxy)piperidin-1-yl)methyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide was purified by semi-preparative reverse phase HPLC (neutral condition; column: Waters Xbridge C18 150*50 mm*10 um; mobile phase: [water (10 mM NH4HCO3)-ACN]; B %: 52%-82%, 10 min). The crude (2S,4R)-1-((2R)-2-(3-(4-((4-((1r,3r)-3-((4-(2-(3-amino-6-(2-hydroxyphenyl)pyridazin-4-yl)-2-fluoroethyl)pyridin-2-yl)oxy)cyclobutoxy)piperidin-1-yl)methyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (Exemplary Compound 2) was purified by semi-preparative reverse phase HPLC (neutral condition; column: Waters Xbridge C18 150*50 mm*10 um; mobile phase: [water (10 mM NH4HCO3)-ACN]; B %: 42%-72%, 10 min). Compound (2S,4R)-1-((R)-2-(3-(4-((4-((1r,3r)-3-((4-(2-(3-amino-6-(2-hydroxyphenyl)pyridazin-4-yl)-2-fluorovinyl)pyridin-2-yl)oxy)cyclobutoxy)piperidin-1-yl)methyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (14.3 mg, 0.01 mmol, 8% yield) and (2S,4R)-1-((2R)-2-(3-(4-((4-((1r,3r)-3-((4-(2-(3-amino-6-(2-hydroxyphenyl)pyridazin-4-yl)-2-fluoroethyl)pyridin-2-yl)oxy)cyclobutoxy)piperidin-1-yl)methyl)piperidin-1-yl)isoxazol-5-yl)-3-methylbutanoyl)-4-hydroxy-N—((S)-1-(4-(4-methylthiazol-5-yl)phenyl)ethyl)pyrrolidine-2-carboxamide (Exemplary Compound 2; 6.9 mg, 0.006 mmol, 3% yield) were obtained as white solids.

### Exemplary Synthesis of Compound 3

### Step 1

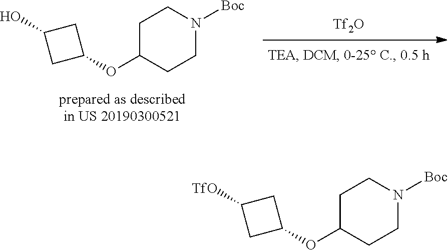 [SMILES: "Tf2O" ; CC(C)(C)OC(=O)N1CCC(O[C@H]2C[C@@H](O)C2)CC1 ; CC(C)(C)OC(=O)N1CCC(O[C@H]2C[C@@H](OS(=O)(=O)C(F)(F)F)C2)CC1]

To a solution of tert-butyl 4-(3-hydroxycyclobutoxy)piperidine-1-carboxylate (4 g, 14.74 mmol, 1 eq) and triethylamine (4.47 g, 44.22 mmol, 6.16 mL, 3 eq) in dichloromethane (120 mL) was added trifluoromethanesulfonic anhydride (4.57 g, 16.22 mmol, 2.68 mL, 1.1 eq) at 0° C. The mixture was stirred at 25° C. for 0.5 hours. The reaction was quenched with water (20 mL). The solution was extracted with dichloromethane (20 mL×2). The organic layers were combined, dried over anhydrous sodium sulfate, filtered and concentrated under reduced pressure to give a residue. The residue was purified by silica gel chromatography (2-5% ethyl acetate in petroleum ether). Compound tert-butyl 4-[3-(trifluoromethylsulfonyloxy)cyclobutoxy]piperidine-1-carboxylate (2.5 g, 6.20 mmol, 42% yield) was obtained as a yellow solid.

### Step 2

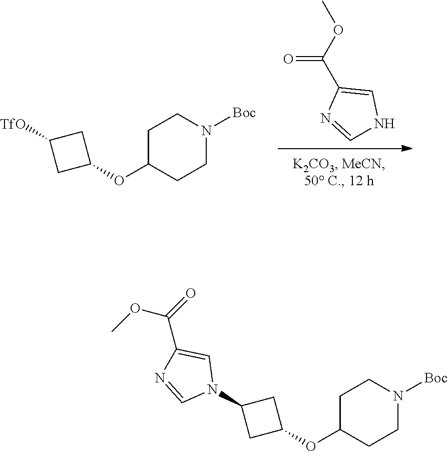

To a tert-butyl 4-[3-(trifluoromethylsulfonyloxy)cyclobutoxy]piperidine-1-carboxylate (700 mg, 1.74 mmol, 1 eq) and methyl 1H-imidazole-4-carboxylate (263 mg, 2.08 mmol, 1.2 eq) in acetonitrile (14 mL) was added potassium carbonate (480 mg, 3.47 mmol, 2 eq). The reaction mixture was stirred at 50° C. for 12 hours. The mixture was cooled to 25° C. and diluted with water (40 mL), and organic layer was extracted with ethyl acetate (60 mL×2). The organic layer was dried over anhydrous sodium sulfate, filtered and concentrated under vacuum. The residue was purified by prep-HPLC (column: Waters Xbridge C18 150*50 mm*10 um; mobile phase: [water (0.05% ammonia hydroxide)-ACN]; B %: 32%-62%). tert-Butyl 4-[3-(4-methoxycarbonylimidazol-1-yl)cyclobutoxy]piperidine-1-carboxylate (120 mg, 316.25 umol, 18.23% yield) was obtained as a light yellow gum.

### Step 3

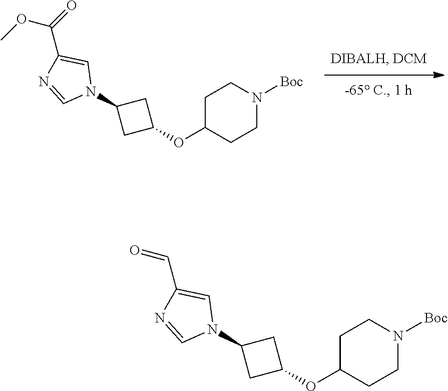 [SMILES: COC(=O)c1cn([C@H]2C[C@H](OC3CCN(C(=O)OC(C)(C)C)CC3)C2)cn1 ; CC(C)(C)OC(=O)N1CCC(O[C@H]2C[C@H](n3cnc(C=O)c3)C2)CC1]

To a solution of tert-butyl 4-[3-(4-methoxycarbonylimidazol-1-yl)cyclobutoxy]piperidine-1-carboxylate (120 mg, 0.32 mmol, 1 eq) in dichloromethane (3 mL) was added diisobutylaluminum hydride (1 M, 0.4 mL, 1.2 eq) dropwise at −65° C. The reaction mixture was stirred at −65° C. for 1 hour. The mixture was quenched with methanol (1 mL) at −65° C. and diluted with dichloromethane (20 mL), then saturated sodium potassium tartrate solution (20 mL) was added. The mixture was stirred at 20° C. for 3 hours. The mixture was extracted with dichloromethane (20 mL). The organic layer was dried over anhydrous sodium sulfate, filtered and concentrated under vacuum. The crude product was used in the next step directly without any further purification. tert-Butyl 4-[3-(4-formylimidazol-1-yl)cyclobutoxy]piperidine-1-carboxylate (105 mg, crude) was obtained as a colorless gum.

### Step 4

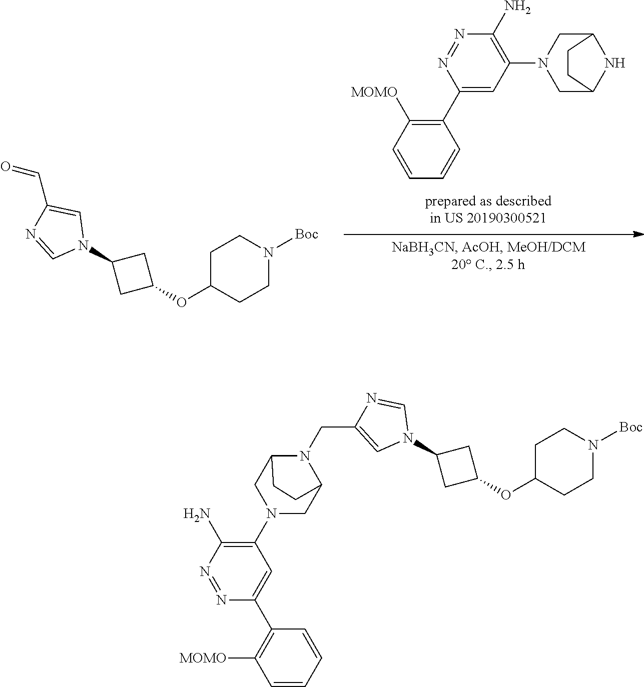

To a solution of tert-butyl 4-[3-(4-formylimidazol-1-yl)cyclobutoxy]piperidine-1-carboxylate (105 mg, 0.30 mmol, 1 eq) and 4-(3,8-diazabicyclo[3.2.1]octan-3-yl)-6-[2-(methoxymethoxy) phenyl]pyridazin-3-amine (72 mg, 0.21 mmol, 0.7 eq) in methanol (3 mL) and dichloromethane (1 mL) was added acetic acid (2 mg, 0.03 mmol, 0.1 eq), abd the solution was stirred at 20° C. for 0.5 hours. To the solution was added sodium cyanoborohydride (57 mg, 901.48 umol, 3 eq). The reaction mixture was stirred at 20° C. for 2 h. The mixture was concentrated under vacuum. The residue was purified by prep-HPLC (column: Waters Xbridge C18 150*50 mm*10 um; mobile phase: [water (10 mM NH4HCO3)-ACN]; B %: 34%-64%). tert-butyl 4-[3-[4-[[3-[3-amino-6-[2-(methoxymethoxy)phenyl]pyridazin-4-yl]-3,8-diazabicyclo[3.2.1]octan-8-yl]methyl]imidazol-1-yl]cyclobutoxy]piperidine-1-carboxylate (37 mg, 0.05 mmol) was obtained as a white solid.

tert-Butyl 4-((1r,3r)-3-(4-((3-(3-amino-6-(2-(methoxymethoxy)phenyl)pyridazin-4-yl)-3,8-diazabicyclo[3.2.1]octan-8-yl)methyl)-1H-imidazol-1-yl)cyclobutoxy)piperidine-1-carboxylate was converted to the title compound according to the scheme below using procedures described above.

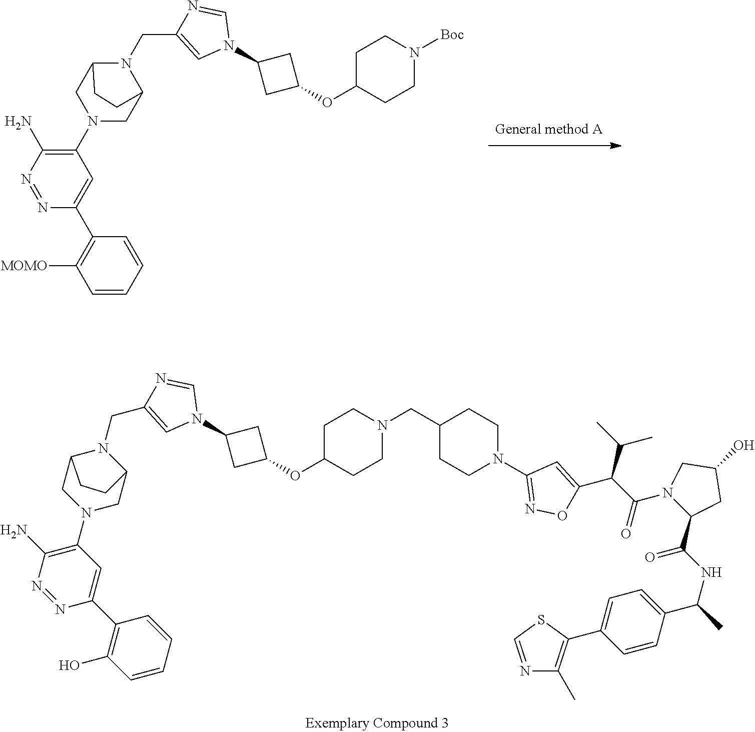

### Exemplary Synthesis of Compound 4

### Step 1

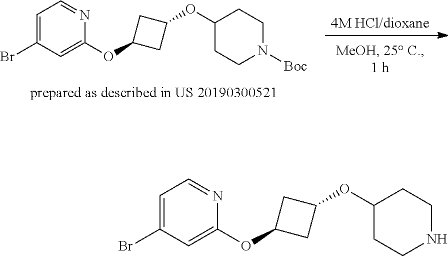 [SMILES: CC(C)(C)OC(=O)N1CCC(O[C@H]2C[C@H](Oc3cc(Br)ccn3)C2)CC1 ; "4M HCl/dioxane" ; Brc1ccnc(O[C@H]2C[C@H](OC3CCNCC3)C2)c1]

To a solution of tert-butyl 4-[3-[(4-bromo-2-pyridyl)oxy]cyclobutoxy]piperidine-1-carboxylate (2 g, 4.68 mmol, 1 eq) in methanol (3 mL) was added hydrogen chloride/dioxane (4 M, 20 mL, 17.09 eq). The mixture was stirred at 25° C. for 1 hour. The reaction mixture was concentrated under reduced pressure. The residue was used in the next step without purification. 4-Bromo-2-[3-(4-piperidyloxy)cyclobutoxy]pyridine hydrochloride (1.7 g, 4.67 mmol) was obtained as a white solid.

### Step 2

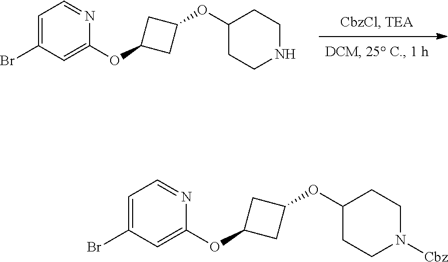 [SMILES: Brc1ccnc(O[C@H]2C[C@H](OC3CCNCC3)C2)c1 ; "CbzCl, TEA" ; O=C(OCc1ccccc1)N1CCC(O[C@H]2C[C@H](Oc3cc(Br)ccn3)C2)CC1]

To a solution of 4-bromo-2-[3-(4-piperidyloxy)cyclobutoxy]pyridine hydrochloride (1.7 g, 4.67 mmol, 1 eq) in dichloromethane (20 mL) was added trifluoroacetic acid (2.37 g, 23.4 mmol, 3.3 mL, 5 eq) and benzyl chloroformate (1.20 g, 7.01 mmol, 1.0 mL, 1.5 eq). The mixture was stirred at 25° C. for 1 hour. 30 mL of water was added to the mixture, and the mixture was extracted with dichloromethane (30 mL×3). The combined organic phase was washed with brine (20 mL×2), dried with anhydrous sodium sulfate, filtered and concentrated in vacuum. The residue was purified by silica gel chromatography (petroleum ether/ethyl acetate=30/1 to 5/1). Benzyl 4-[3-[(4-bromo-2-pyridyl)oxy]cyclobutoxy]piperidine-1-carboxylate (2 g, 3.90 mmol) was obtained as a colorless oil.

### Step 3

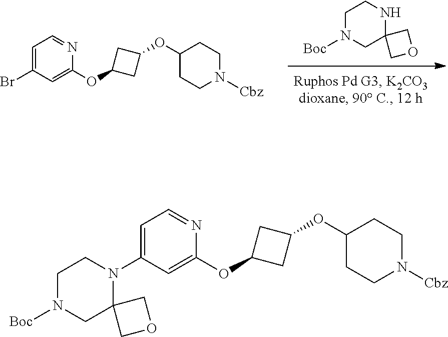 [SMILES: O=C(OCc1ccccc1)N1CCC(O[C@H]2C[C@H](Oc3cc(Br)ccn3)C2)CC1 ; CC(C)(C)OC(=O)N1CCNC2(COC2)C1 ; CC(C)(C)OC(=O)N1CCN(c2ccnc(O[C@H]3C[C@H](OC4CCN(C(=O)OCc5ccccc5)CC4)C3)c2)C2(COC2)C1]

To a solution of benzyl 4-[3-[(4-bromo-2-pyridyl)oxy]cyclobutoxy]piperidine-1-carboxylate (250 mg, 0.54 mmol, 1 eq) and tert-butyl 2-oxa-5,8-diazaspiro[3.5]nonane-8-carboxylate (130 mg, 0.57 mmol, 1.05 eq) in dioxane (5 mL) was added potassium carbonate (149 mg, 1.08 mmol, 2 eq) and methanesulfonato(2-dicyclohexylphosphino-2,6-di-i-propoxy-1,1-biphenyl)(2-amino-1,1-biphenyl-2-yl)palladium(II) (45 mg, 0.05 mmol, 0.1 eq) under nitrogen. The reaction mixture was stirred at 90° C. for 12 hour. The mixture was filtered, and the filtrate was concentrated in vacuum. The residue was purified by prep-TLC (dichloromethane:methanol=15:1). tert-Butyl 5-[2-[3-[(1-benzyloxycarbonyl-4-piperidyl)oxy]cyclobutoxy]-4-pyridyl]-2-oxa-5,8-diazaspiro[3.5]nonane-8-carboxylate (200 mg, 0.33 mmol) was obtained as a light yellow oil.

### Step 4

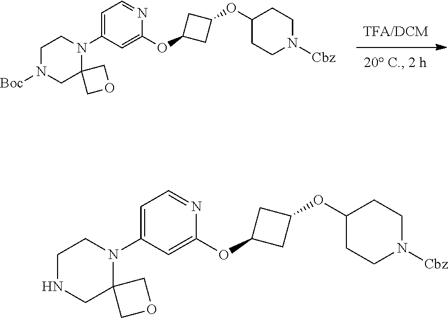 [SMILES: CC(C)(C)OC(=O)N1CCN(c2ccnc(O[C@H]3C[C@H](OC4CCN(C(=O)OCc5ccccc5)CC4)C3)c2)C2(COC2)C1 ; O=C(OCc1ccccc1)N1CCC(O[C@H]2C[C@H](Oc3cc(N4CCNCC45COC5)ccn3)C2)CC1]

To a solution of tert-butyl 5-[2-[3-[(1-benzyloxycarbonyl-4-piperidyl)oxy]cyclobutoxy]-4-pyridyl]-2-oxa-5,8-diazaspiro[3.5]nonane-8-carboxylate (230 mg, 0.38 mmol, 1 eq) in dichloromethane (5 mL) was added trifluoroacetic acid (1.54 g, 13.51 mmol, 1 mL, 35.8 eq). The reaction mixture was stirred at 20° C. for 2 hours. The mixture was concentrated in vacuum. Saturated sodium bicarbonate (50 mL) was added to the mixture. The aqueous phase was extracted with ethyl acetate (30 mL×3). The combined organic phase was washed with brine (30 mL), dried over sodium sulfate, filtered, and concentrated in vacuum. Benzyl 4-[3-[[4-(2-oxa-5,8-diazaspiro[3.5]nonan-5-yl)-2-pyridyl]oxy]cyclobutoxy] piperidine-1-carboxylate (200 mg, crude) was obtained as a yellow oil.

### Step 5

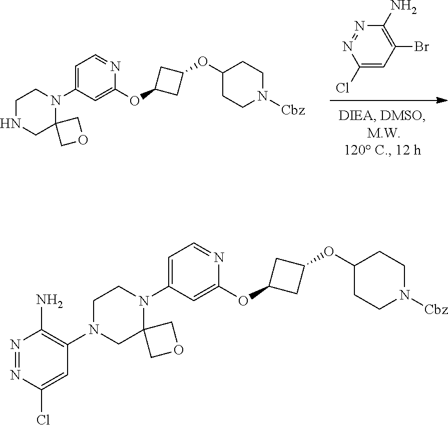 [SMILES: O=C(OCc1ccccc1)N1CCC(O[C@H]2C[C@H](Oc3cc(N4CCNCC45COC5)ccn3)C2)CC1 ; Nc1nnc(Cl)cc1Br ; Nc1nnc(Cl)cc1N1CCN(c2ccnc(O[C@H]3C[C@H](OC4CCN(C(=O)OCc5ccccc5)CC4)C3)c2)C2(COC2)C1]

To a solution of benzyl 4-[3-[[4-(2-oxa-5,8-diazaspiro[3.5]nonan-5-yl)-2-pyridyl]oxy] cyclobutoxy]piperidine-1-carboxylate (200 mg, 0.39 mmol, 1 eq) and 4-bromo-6-chloro-pyridazin-3-amine (410 mg, 1.97 mmol, 5 eq) in dimethylsulfoxide (5 mL) was added N,N-diisopropylethylamine (254 mg, 1.97 mmol, 0.3 mL, 5 eq). The reaction mixture was stirred at 120° C. for 12 hours under microwave irradiation. Water (50 mL) was added to the mixture. The aqueous phase was extracted with ethyl acetate (30 mL×3). The combined organic phase was washed with brine (30 mL×2), dried over sodium sulfate, filtered and concentrated in vacuum. The residue was purified by prep-TLC (ethyl acetate:methanol=15:1). Benzyl 4-[3-[[4-[8-(3-amino-6-chloro-pyridazin-4-yl)-2-oxa-5,8-diazaspiro[3.5]nonan-5-yl]-2-pyridyl]oxy]cyclobutoxy]piperidine-1-carboxylate (130 mg, 0.20 mmol) was obtained as a yellow solid.

### Step 6

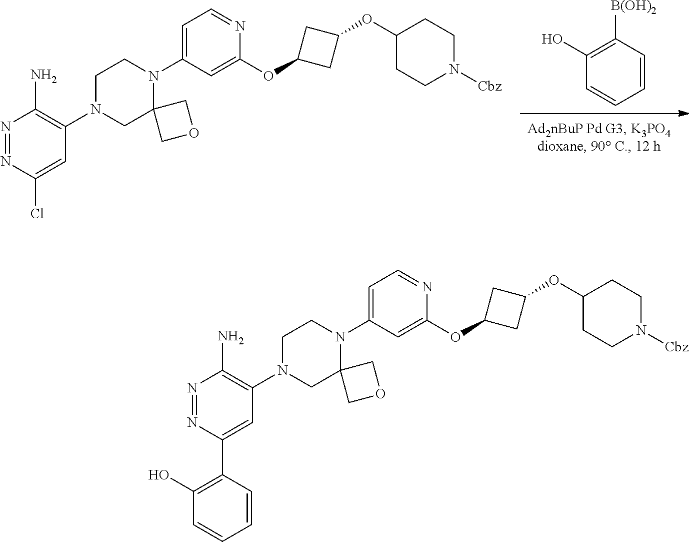 [SMILES: Nc1nnc(Cl)cc1N1CCN(c2ccnc(O[C@H]3C[C@H](OC4CCN(C(=O)OCc5ccccc5)CC4)C3)c2)C2(COC2)C1 ; OB(O)c1ccccc1O ; Nc1nnc(-c2ccccc2O)cc1N1CCN(c2ccnc(O[C@H]3C[C@H](OC4CCN(C(=O)OCc5ccccc5)CC4)C3)c2)C2(COC2)C1]

To a solution of benzyl 4-[3-[[4-[8-(3-amino-6-chloro-pyridazin-4-yl)-2-oxa-5,8-diazaspiro [3.5]nonan-5-yl]-2-pyridyl]oxy]cyclobutoxy]piperidine-1-carboxylate (130 mg, 0.20 mmol, 1 eq) and (2-hydroxyphenyl)boronic acid (56 mg, 0.41 mmol, 2 eq) in dioxane (3 mL) was added potassium phosphate (1.5 M, 0.4 mL, 3 eq) and methanesulfonato (diadamantyl-n-butylphosphino)-2-amino-1,1-biphenyl-2-yl)palladium(II) dichloromethane adduct (15 mg, 0.02 mmol, 0.1 eq) under nitrogen. The reaction mixture was stirred at 90° C. for 12 hours. Ethyl acetate (20 mL) was added to the mixture, and the mixture was dried over sodium sulfate, filtered, and concentrated in vacuum. The residue was purified by prep-TLC (dichloromethane:methanol=10:1). Benzyl 4-[3-[[4-[8-[3-amino-6-(2-hydroxyphenyl)pyridazin-4-yl]-2-oxa-5,8-diazaspiro[3.5]nonan-5-yl]-2-pyridyl]oxy]cyclobutoxy]piperidine-1-carboxylate (100 mg, 0.14 mmol) was obtained as a yellow solid.

Benzyl 4-[3-[[4-[8-[3-amino-6-(2-hydroxyphenyl)pyridazin-4-yl]-2-oxa-5,8-diazaspiro[3.5]nonan-5-yl]-2-pyridyl]oxy]cyclobutoxy]piperidine-1-carboxylate was converted to the title compound according to the scheme below using procedures described above.

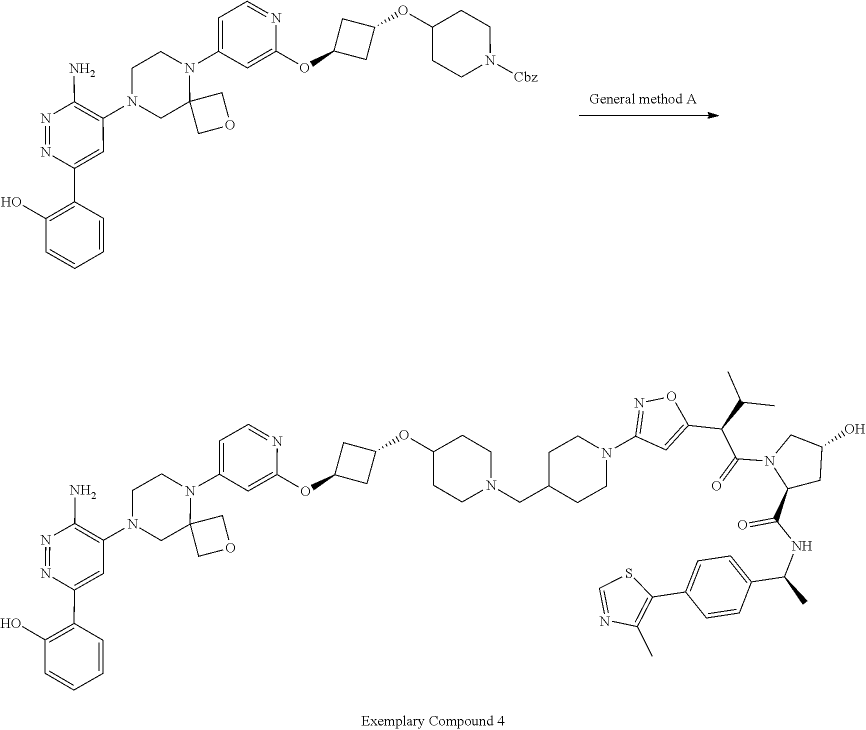 [SMILES: Nc1nnc(-c2ccccc2O)cc1N1CCN(c2ccnc(O[C@H]3C[C@H](OC4CCN(C(=O)OCc5ccccc5)CC4)C3)c2)C2(COC2)C1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Oc6cc(N7CCN(c8cc(-c9ccccc9O)nnc8N)CC78COC8)ccn6)C5)CC4)CC3)no2)C(C)C)cc1]

Using analogous procedures the Compounds 6, 23, 24, and 25 were prepared.

### Exemplary Synthesis of Compound 5

### Step 1

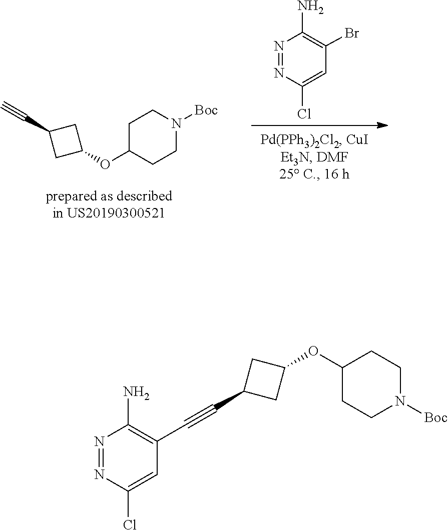 [SMILES: C#C[C@H]1C[C@H](OC2CCN(C(=O)OC(C)(C)C)CC2)C1 ; Nc1nnc(Cl)cc1Br ; CC(C)(C)OC(=O)N1CCC(O[C@H]2C[C@H](C#Cc3cc(Cl)nnc3N)C2)CC1]

A mixture of tert-butyl 4-(3-ethynylcyclobutoxy)piperidine-1-carboxylate (2 g, 7.16 mmol, 1 eq), 4-bromo-6-chloro-pyridazin-3-amine (2.24 g, 10.74 mmol, 1.5 eq), bis(triphenylphosphine)palladium(II) dichloride (502 mg, 0.72 mmol, 0.1 eq), cuprous iodide (136 mg, 0.72 mmol, 0.1 eq) and triethylamine (2.17 g, 21.48 mmol, 3.0 mL, 3 eq) in N,N-dimethylformamide (15 mL) was stirred at 25° C. for 16 hours under nitrogen. The reaction mixture was filtered and concentrated under reduced pressure. The residue was purified by silica gel column chromatography (petroleum ether/ethyl acetate=2/1). Then it was further purified by preparative HPLC (column: Waters Xbridge BEH C18 250*50 mm*10 um; mobile phase: [water (0.05% ammonia hydroxide v/v)-ACN]; B %: 38%-65%). Compound tert-butyl 4-[3-[2-(3-amino-6-chloro-pyridazin-4-yl)ethynyl]cyclobutoxy] piperidine-1-carboxylate (1.5 g, 3.69 mmol) was obtained as a light yellow solid.

### Step 2

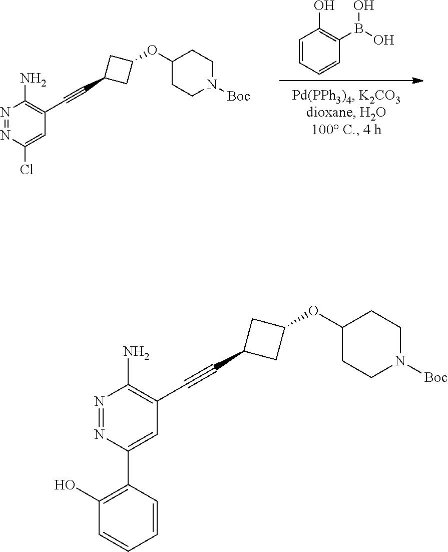 [SMILES: CC(C)(C)OC(=O)N1CCC(O[C@H]2C[C@H](C#Cc3cc(Cl)nnc3N)C2)CC1 ; OB(O)c1ccccc1O ; CC(C)(C)OC(=O)N1CCC(O[C@H]2C[C@H](C#Cc3cc(-c4ccccc4O)nnc3N)C2)CC1]

A mixture of tert-butyl 4-[3-[2-(3-amino-6-chloro-pyridazin-4-yl)ethynyl]cyclobutoxy] piperidine-1-carboxylate (1.4 g, 3.44 mmol, 1 eq), (2-hydroxyphenyl)boronic acid (949 mg, 6.88 mmol, 2 eq), tetrakis[triphenylphosphine]palladium(0) (398 mg, 0.34 mmol, 0.1 eq) and potassium carbonate (1.43 g, 10.32 mmol, 3 eq) in dioxane (30 mL) and water (3 mL) was stirred at 100° C. for 4 hours under nitrogen. The reaction mixture was filtered and concentrated under reduced pressure. The residue was purified by silica gel column chromatography (petroleum ether/ethyl acetate=3/1 to 1/1), and then additionally purified by preparative HPLC (column: Waters Xbridge BEH C18 250*50 mm*10 um; mobile phase: [water (0.05% ammonia hydroxide v/v)-ACN]; B %: 45%-75%). Compound tert-butyl 4-[3-[2-[3-amino-6-(2-hydroxyphenyl)pyridazin-4-yl]ethynyl]cyclobutoxy] piperidine-1-carboxylate (740 mg, 1.59 mmol, 46% yield) was obtained as a yellow solid.

tert-Butyl 4-[3-[2-[3-amino-6-(2-hydroxyphenyl)pyridazin-4-yl]ethynyl]cyclobutoxy] piperidine-1-carboxylate was converted to the title compound according to the scheme below using procedures described above.

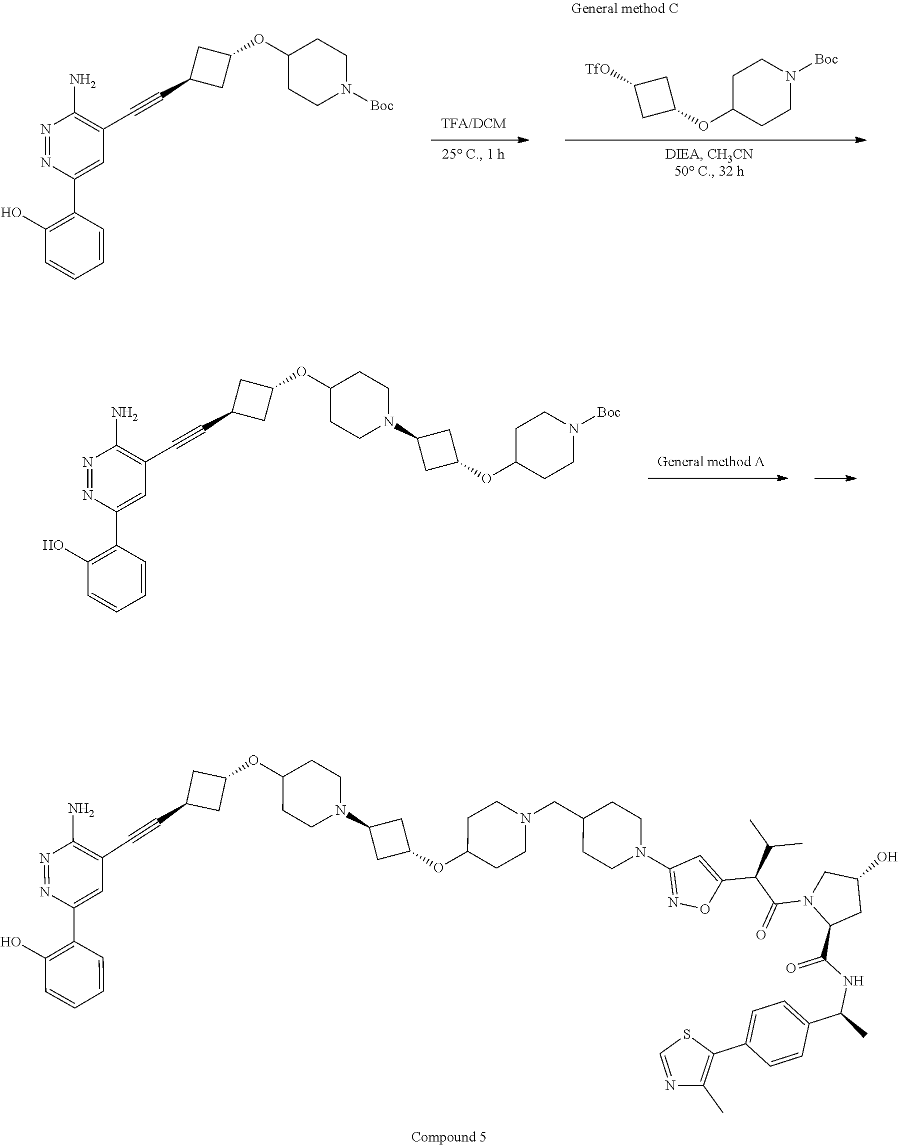 [SMILES: CC(C)(C)OC(=O)N1CCC(O[C@H]2C[C@H](C#Cc3cc(-c4ccccc4O)nnc3N)C2)CC1 ; CC(C)(C)OC(=O)N1CCC(O[C@H]2C[C@@H](OS(=O)(=O)C(F)(F)F)C2)CC1 ; CC(C)(C)OC(=O)N1CCC(O[C@H]2C[C@H](N3CCC(O[C@H]4C[C@H](C#Cc5cc(-c6ccccc6O)nnc5N)C4)CC3)C2)CC1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(O[C@H]7C[C@H](C#Cc8cc(-c9ccccc9O)nnc8N)C7)CC6)C5)CC4)CC3)no2)C(C)C)cc1]

### Exemplary Synthesis of Compound 7

Prepared according to the scheme below using procedures described for other Examples above as well as procedures commonly known to those skilled in the art.

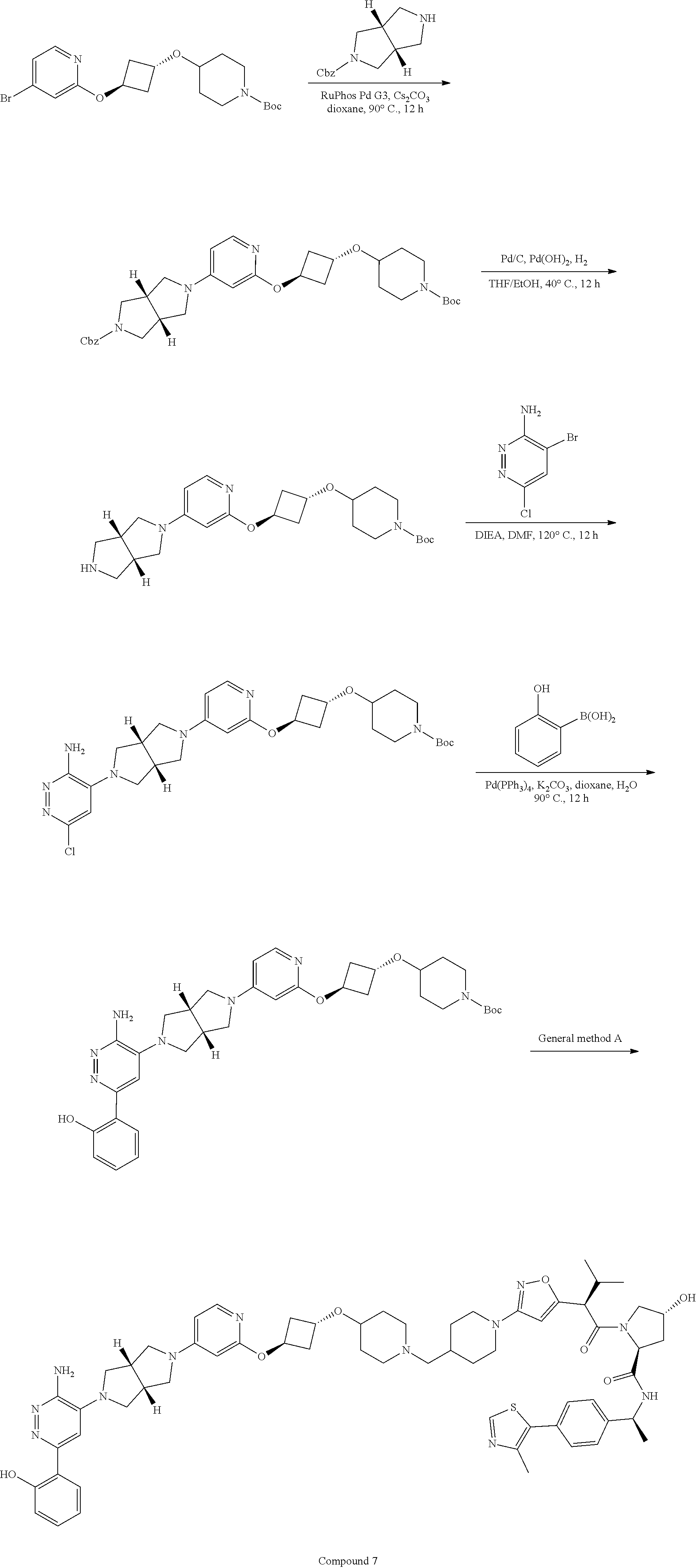

### Exemplary Synthesis of Compound 8

Prepared according to the scheme below using procedures described for other Examples above as well as procedures commonly known to those skilled in the art.

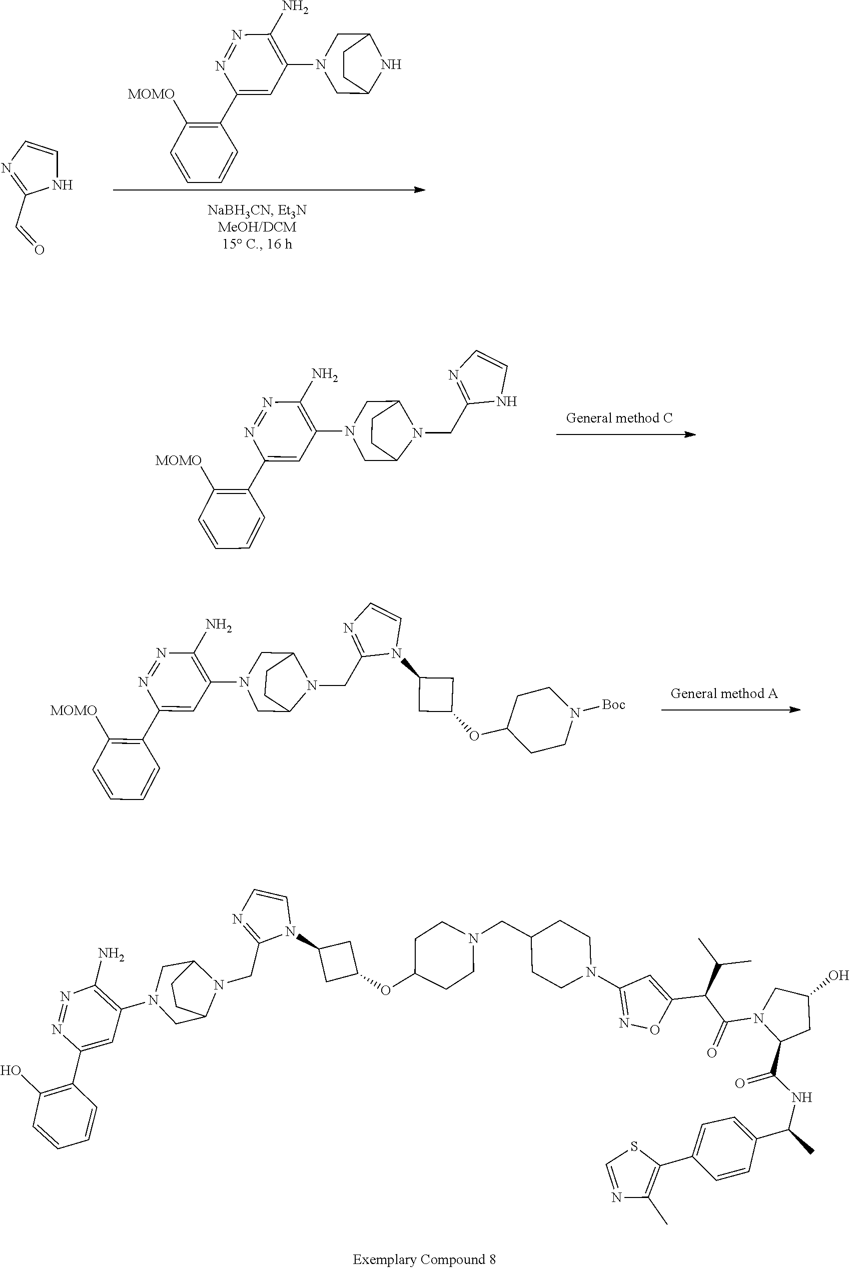

### Exemplary Synthesis of Compound 9

Prepared according to the scheme below using procedures described for Exemplary Compounds 4 and 7, and other Examples above as well as procedures commonly known to those skilled in the art.

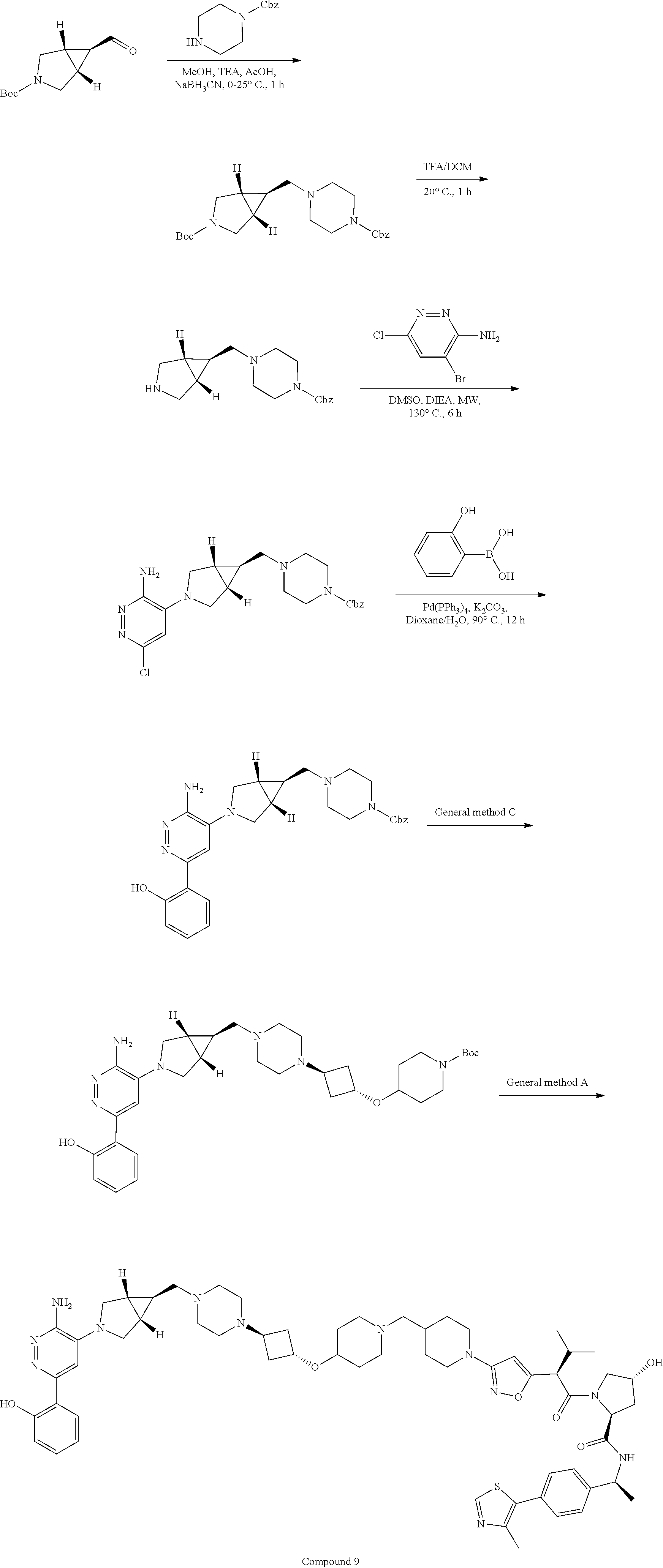

Using analogous procedures Compound 13 was prepared.

### Exemplary Synthesis of Compound 10

### Step 1

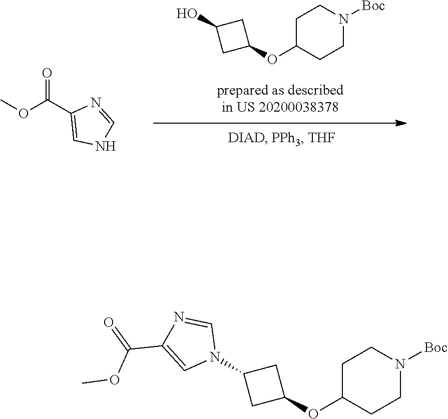

A solution of methyl 1H-imidazole-4-carboxylate (883 mg, 7.00 mmol, 1.0 eq), tert-butyl 4-(3-hydroxycyclobutoxy)piperidine-1-carboxylate (1.9 g, 7.00 mmol, 1.0 eq), DIAD (1.7 g, 8.40 mmol, 1.63 mL, 1.2 eq) and PPh₃ (2.2 g, 8.40 mmol, 1.2 eq) in tetrahydrofuran (20 mL) was stirred at 55° C. for 16 hours. The mixture was concentrated. The crude product was purified by Biotage® combi flash (Column: 40 g Biotage® Silica Flash column; Eluent: from 70-100% methyl tert-butyl ether in petroleum ether to 7% methanol in methyl tert-butyl ether). tert-Butyl 4-[3-(4-methoxycarbonylimidazol-1-yl)cyclobutoxy]piperidine-1-carboxylate (945 mg, 2.33 mmol) was obtained as a yellow oil.

### Step 2

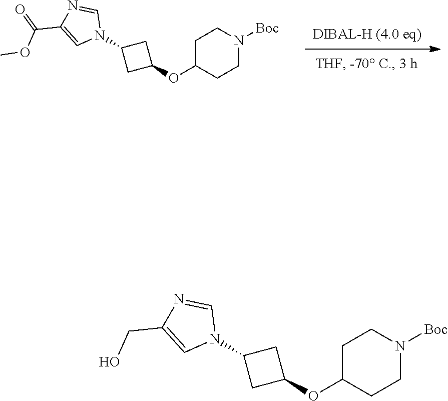 [SMILES: COC(=O)c1cn([C@H]2C[C@H](OC3CCN(C(=O)OC(C)(C)C)CC3)C2)cn1 ; CC(C)(C)OC(=O)N1CCC(O[C@H]2C[C@H](n3cnc(CO)c3)C2)CC1]

To a solution of tert-butyl 4-[3-(4-methoxycarbonylimidazol-1-yl)cyclobutoxy]piperidine-1-carboxylate (945 mg, 2.49 mmol, 1.0 eq) in dichloromethane (15 mL) was added DIBAL-H (1 M, 12.5 mL, 5.0 eq) dropwise at −70° C. over 10 minutes. Then the mixture was allowed to warm up and stirred at 20° C. for 16 hours. The mixture was quenched with methanol (30 mL) at 0° C. and concentrated. To the residue was added dichloromethane (50 mL) and tartrate (50 mL), and the mixture was stirred at 20° C. for 15 minutes. The mixture was filtered, extracted with dichloromethane (50 mL×3). The combined organic layer was washed with brine (20 mL), dried over Na₂SO₄, filtered and concentrated to give tert-butyl 4-[3-[4-(hydroxymethyl)imidazol-1-yl]cyclobutoxy]piperidine-1-carboxylate (450 mg, 1.11 mmol) as a yellow gum.

### Step 3

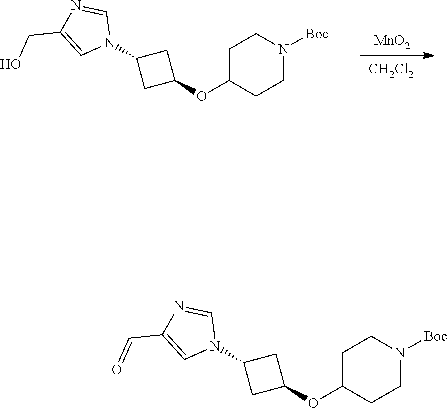

To a solution of tert-butyl 4-[3-[4-(hydroxymethyl)imidazol-1-yl]cyclobutoxy]piperidine-1-carboxylate (450 mg, 1.28 mmol, 1.0 eq) in dichloromethane (10 mL) was added MnO₂ (1.10 g, 12.80 mmol, 10 eq). The mixture was stirred at 20° C. for 16 hours. The mixture was filtered and concentrated. The crude product was purified by Biotage® combi flash (Column: 4 g Biotage® Silica Flash column; Eluent: gradient 0-2% methanol in dichloromethane). tert-Butyl 4-[3-(4-formylimidazol-1-yl)cyclobutoxy]piperidine-1-carboxylate (300 mg, 0.77 mmol) was obtained as a light yellow solid.

tert-Butyl 4-[3-(4-formylimidazol-1-yl)cyclobutoxy]piperidine-1-carboxylate was converted to the title compound as described for other Examples above.

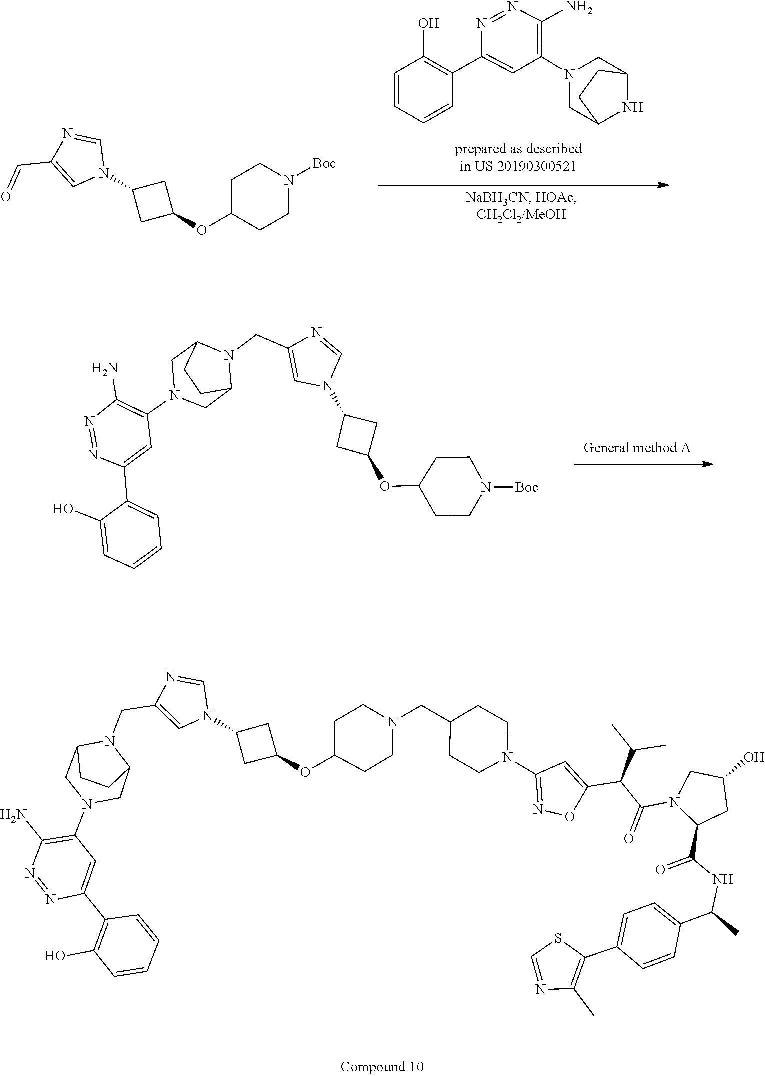 [SMILES: Nc1nnc(-c2ccccc2O)cc1N1CC2CCC(C1)N2 ; CC(C)(C)OC(=O)N1CCC(O[C@H]2C[C@H](n3cnc(C=O)c3)C2)CC1 ; CC(C)(C)OC(=O)N1CCC(O[C@H]2C[C@H](n3cnc(CN4C5CCC4CN(c4cc(-c6ccccc6O)nnc4N)C5)c3)C2)CC1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](n6cnc(CN7C8CCC7CN(c7cc(-c9ccccc9O)nnc7N)C8)c6)C5)CC4)CC3)no2)C(C)C)cc1]

Using analogous procedures Compounds 12 and 14 were prepared.

### Exemplary Synthesis for Compound 11

Compound 11 was prepared according to the scheme below using procedures described for Compounds 4, 7, and 9, and other Examples above as well as procedures commonly known to those skilled in the art.

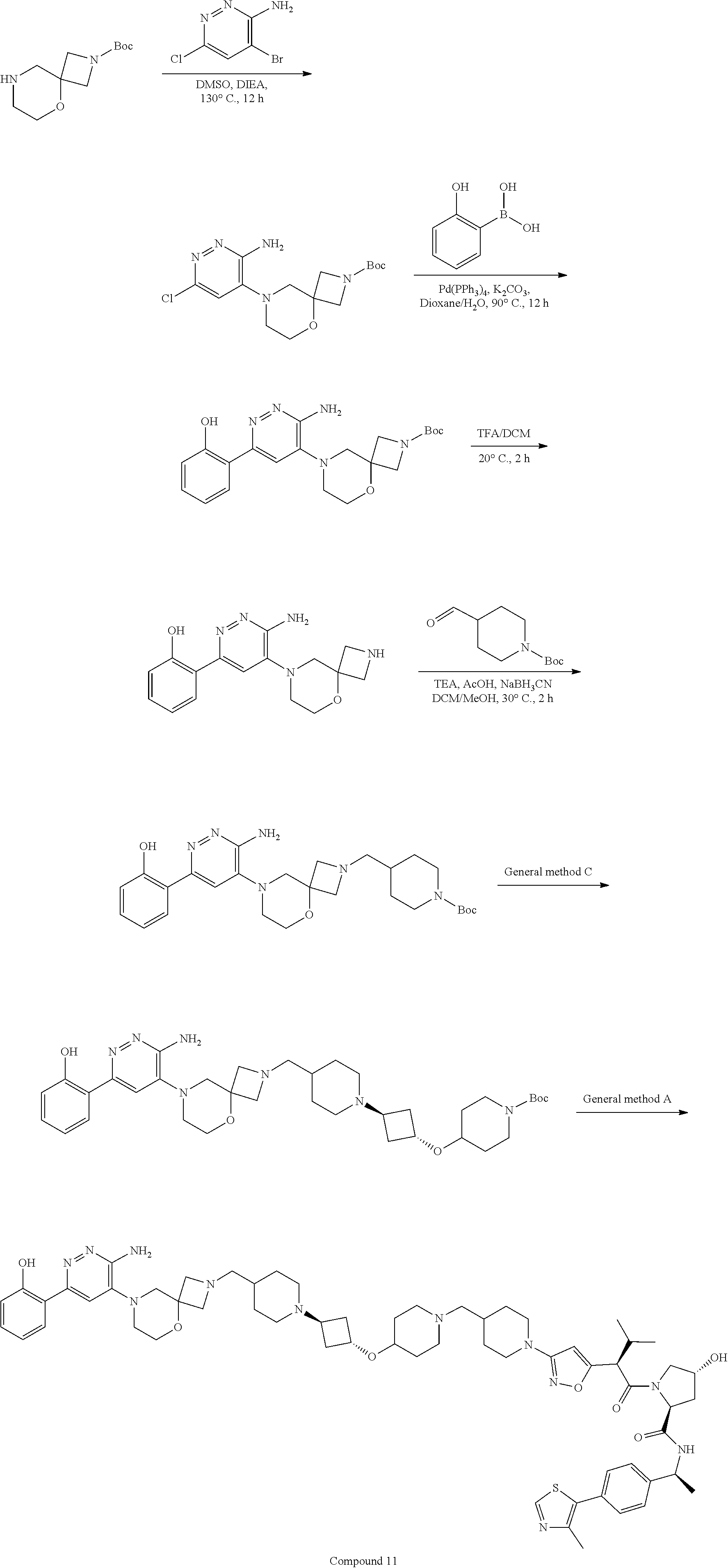

Using analogous procedures Compound 27 was prepared.

### Exemplary Synthesis of Compound 15

Compound 15 was prepared as described in the scheme below.

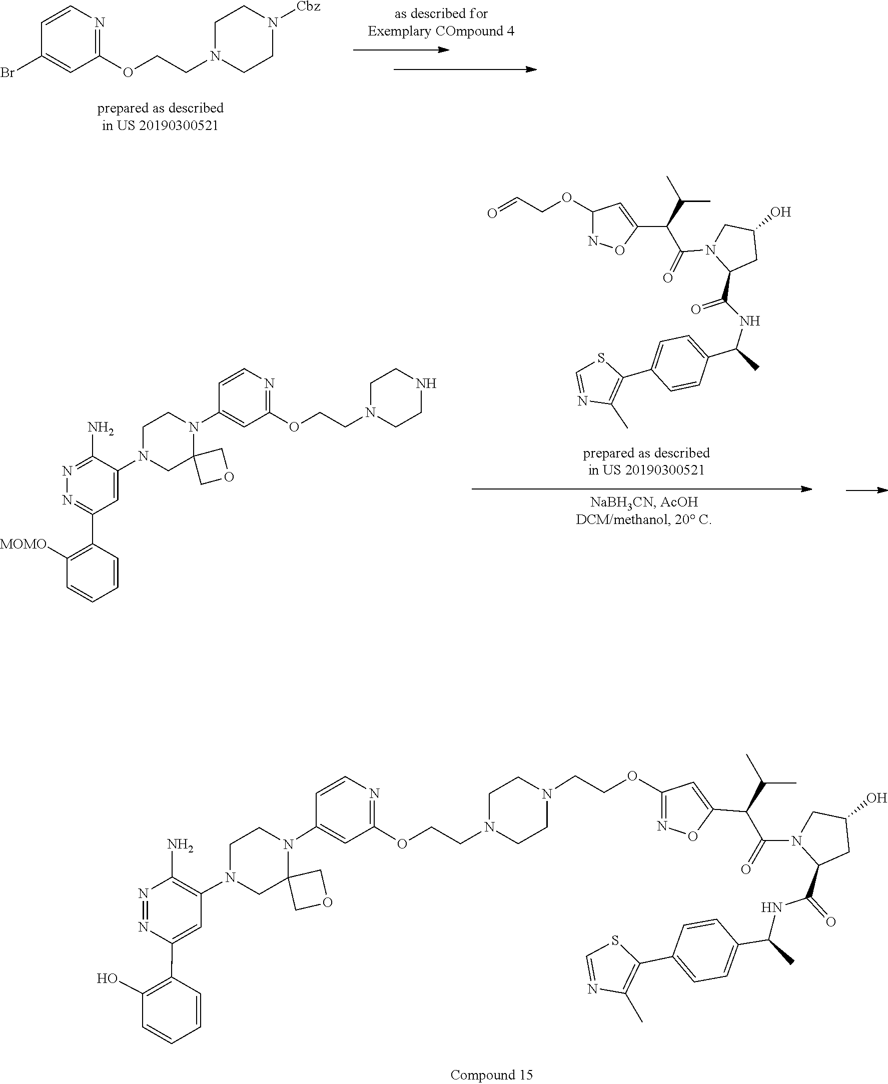

### Exemplary Synthesis of Compound 16 and Compound 17

Prepared according to the scheme below using procedures described for other Examples above.

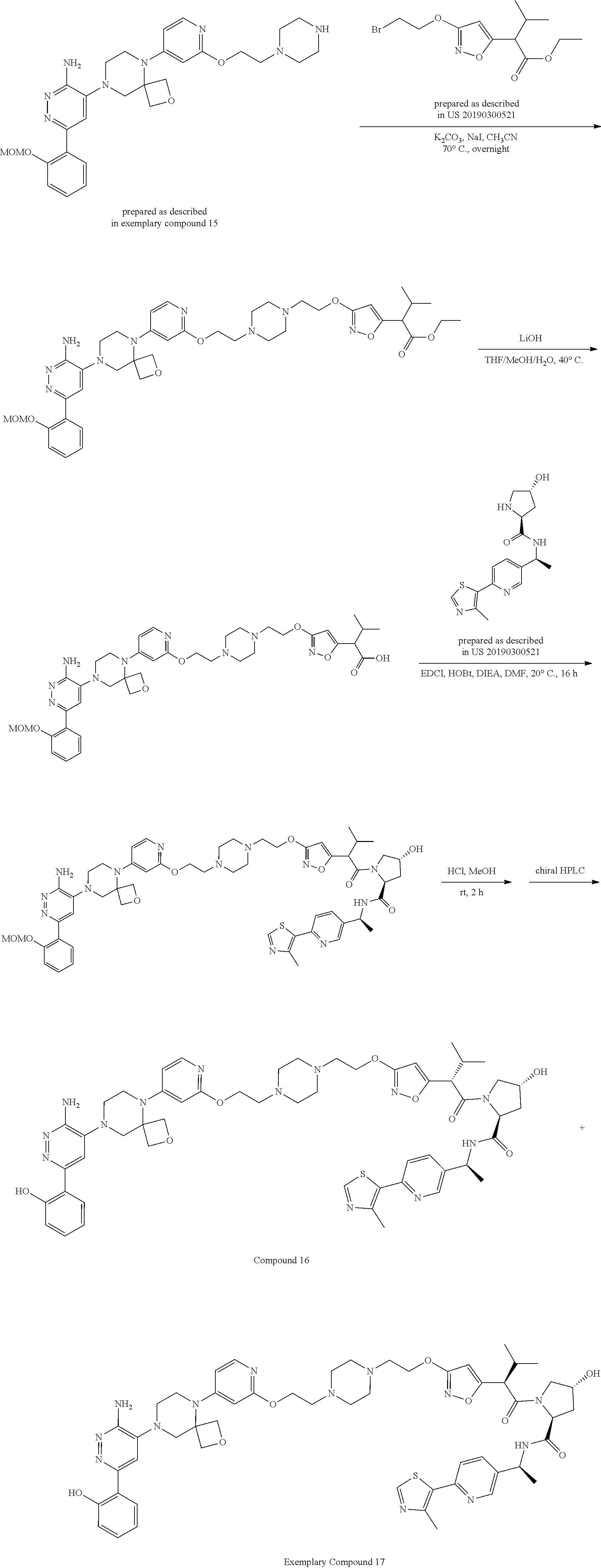

Using analogous procedures Compounds 19, 20, and 21 were prepared.

### Exemplary Synthesis of Compound 18

Compound 18 was prepared according to the schemes below using procedures described for other Examples above as well as general procedures commonly known to those skilled in the art.

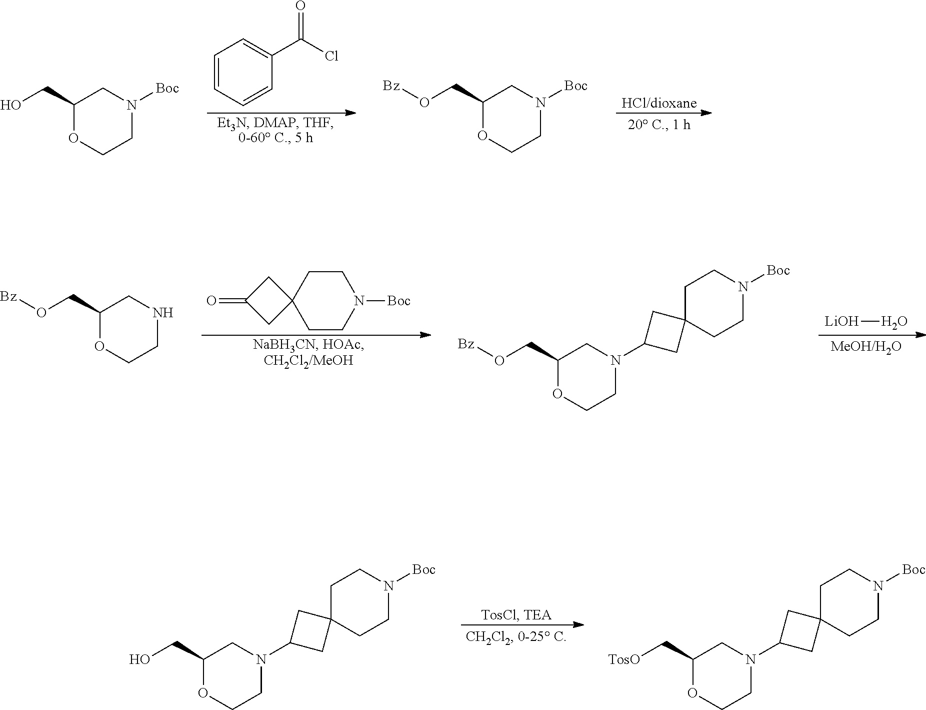

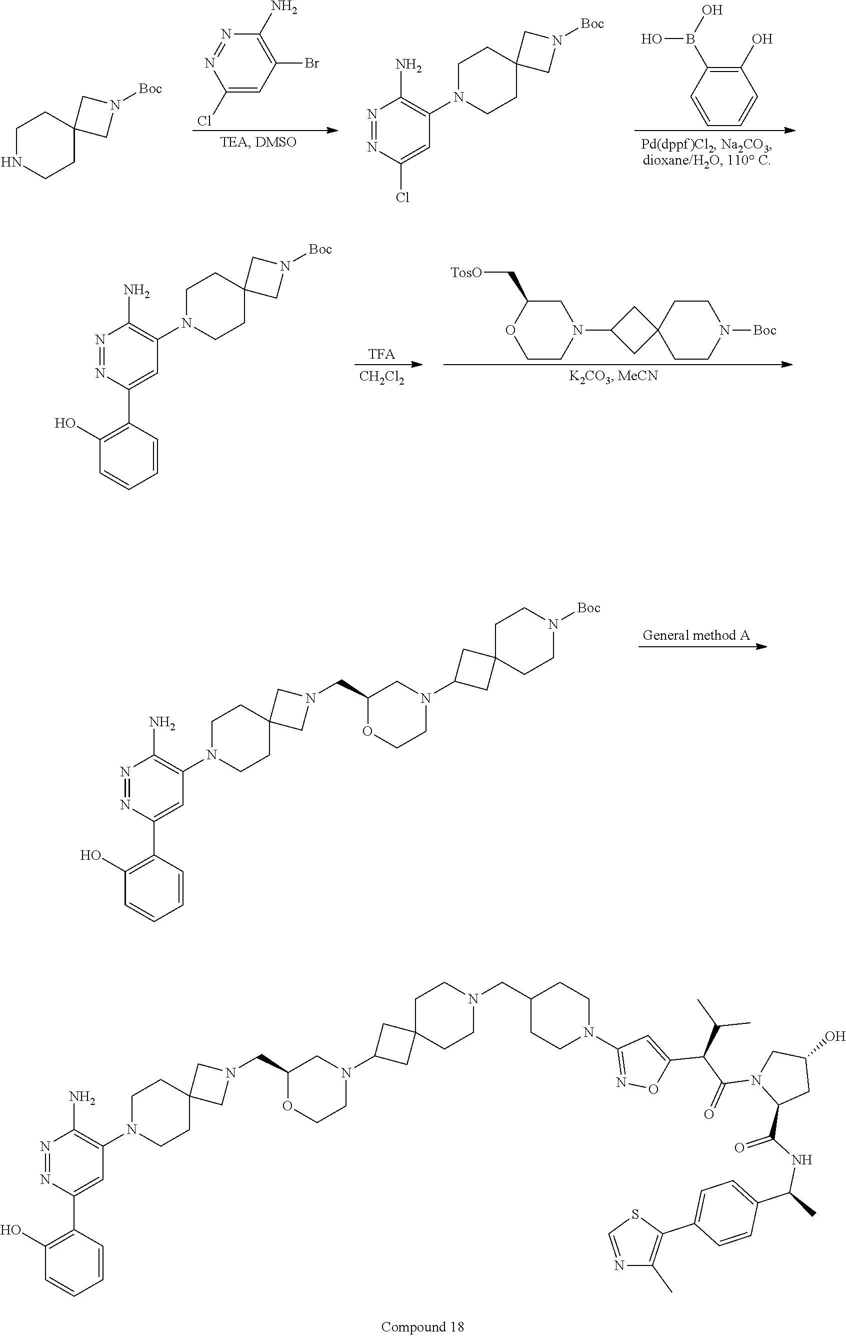

### Exemplary Synthesis of Compound 22

Compound 22 was prepared according to the schemes below using procedures described for other Examples.

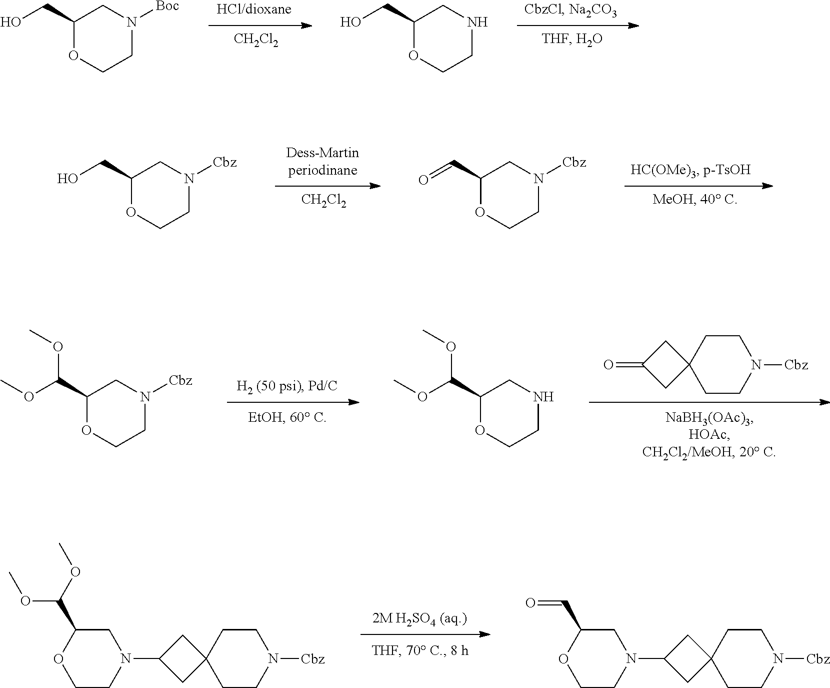

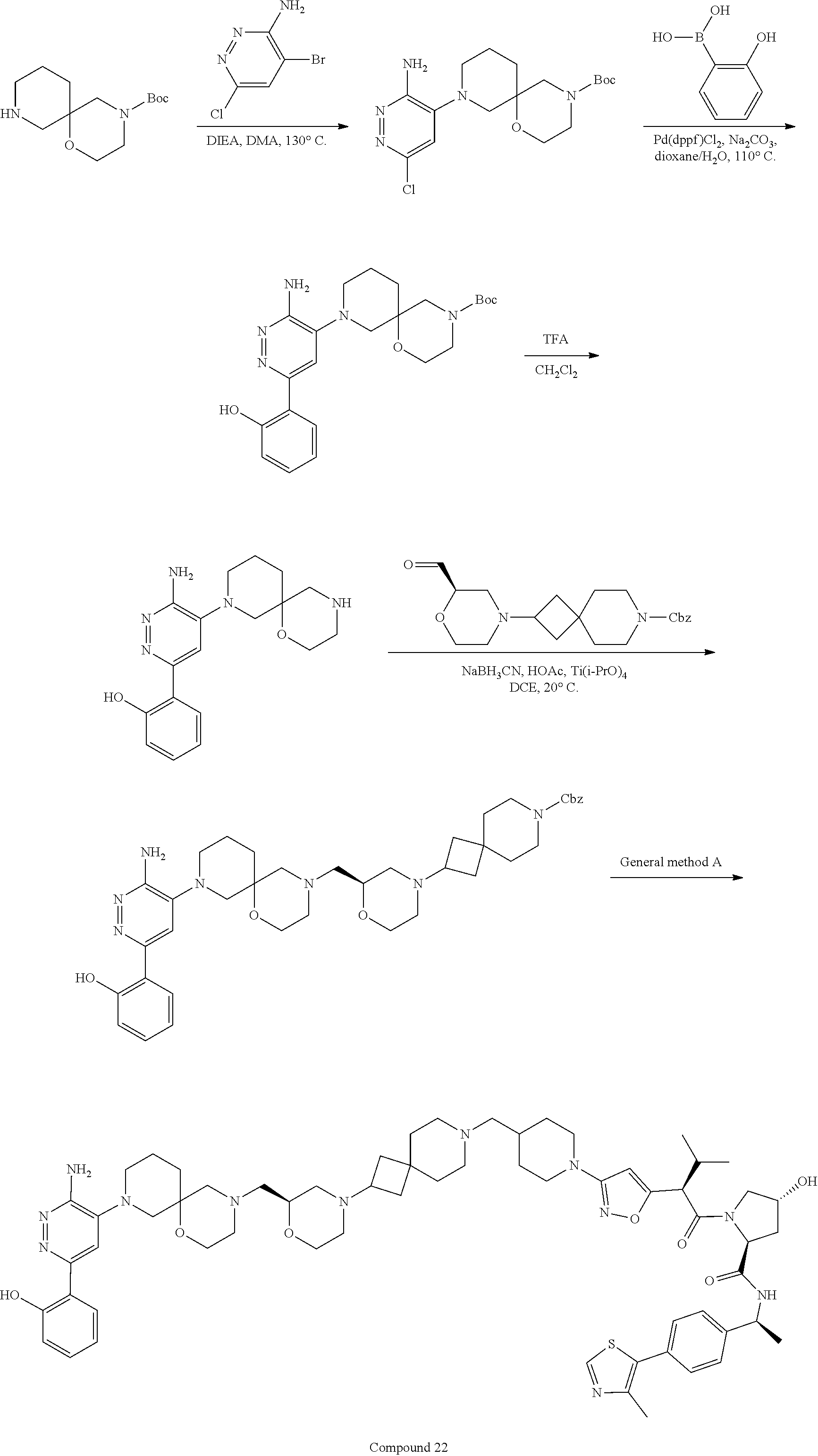

Using analogous procedures Compound 26 was prepared.

### AlphaLISA Competitive BRM Binding Assay

Compounds were serially diluted in 100% DMSO in 3-fold increments. An intermediate compound plate was created at 10% DMSO in AlphaLISA buffer (50 mM Hepes, pH 7.5, 50 mM NaCl, 69 mM Brij-35, 0.1 mg/mL BSA) and then 3 mL of compound transferred from the intermediate plate into a white, 384-OptiPlate (Perkin Elmer, #6007290). A solution of protein (recombinant human 6×His-tagged-BRM (bromodomain-only; aa. 1377-1486; NP_620614) expressed in Escherichia coli) and biotinylated probe are mixed and pre-incubated for 10 minutes. Following incubation, 12 mL of protein/probe solution is added to each well of the 384-OptiPlate, resulting in final assay concentration of 7 nM protein (6×His-BRM) and 20 nM biotinylated probe. Compound and protein/probe solution were incubated at room temperature for 10 minutes. 7.5 mL of anti-His conjugated AlphaLISA acceptor beads (PerkinElmer #AL128M), diluted 100× in AlphaLISA buffer, were added to each well of the assay plate. This addition was followed by a 10-minute incubation at room temperature, in the dark. Lastly, 7.5 mL of streptavidin donor beads (PerkinElmer #6760002), diluted 100× in AlphaLISA buffer, were added to each well of the assay plate. Final assay plate was incubated for 15 minutes before read, signal is stable up to 4 hours after donor bead addition.

Assay plates were read using an Envision® multimode microplate reader (PerkinElmer #2105-0010) using the Europium/615 nM emission filter. Data is reported as percent displacement, 100% binding is based on the average fluorescent count (FC) of zero compound column and 0% binding is based on the average fluorescent count (FC) of zero protein (His-BRM) column. Percent displacement is calculated based on the following equation:

((100% Binding FC−Compound Well FC)/(100% Binding FC−0% Binding FC))*100

Data is fit using a 4-parameter non-linear regression analysis in GraphPad Prism.

### Protein Level Control

This description also provides methods for the control of protein levels with a cell. This is based on the use of compounds as described herein, which are known to interact with a specific target protein such that degradation of a target protein in vivo will result in the control of the amount of protein in a biological system, preferably to a particular therapeutic benefit.

The following examples are used to assist in describing the present disclosure, but should not be seen as limiting the present disclosure in any way.

### Assays and Degradation Data

### Western Blot Screen of BRM Degradation in SW1573 Cells

To assess BRM degradation (Dₘₐₓ and DC₅₀) cells were seeded at 8000/well in 96-well black/clear-bottom plates in 180 μL DMEM growth media (containing 1% pen-strep, 1% HEPES and 10% FBS) per well. Plates were incubated overnight to allow adhesion. The next morning cells were treated by adding 20 μL of 10× target compound concentration (1% DMSO) to appropriate wells and returned to incubator for overnight (18-20 hours). The final DMSO concentration was 0.1%.

For lysing, adherent cells were washed once with 100 μL of DPBS. Cells were lysed in 40 μL of 1× RIPA+HALT protease inhibitor on ice for 10 minutes and frozen until use at −80° C. Thawed lysates were cleaned by filtration in 1.2 m filter plates, or alternatively, were spun clean at 2300 g at 4° C. for 30 minutes.

For blotting, for each Western sample 30 μL of lysate was added to 10 μL of 4×LDS sample buffer, then denatured at 95° C. for 5 minutes in the thermal cycler and placed on ice. Samples were loaded on 4-15% Tris/Glycine gels and run for 25 minutes at 250 constant volts in 1×Tris/Glycine buffer with 4 μL of ladder and 12 μL of each sample loaded for each blot. Protein was transferred from gels to NC with BioRad Turbo dry-transfer unit with the Turbo/midi default program. All blots were rinsed with ddH2O and blocked for 1 hour at room temperature in 5% BSA in TBST (0.1%) on rocker. Blots were exposed to primary antibody in 5% BSA in TBST (0.1%) overnight at 4° C. on rocker (1:1000 for BRM (Cell Signaling Tech. cat #11966) and 1:2000 for alpha-tubulin (Cell Signaling Tech. cat #3873), a control protein. Blot was washed with TBST (0.1%) three times for 5 minutes on rocker at room temperature. Secondary antibody was added, and blots were incubated on a room temperature rocker for 1 hour with 1:18,000 anti-rabbit-HRP and/or anti-mouse-HRP in 5% BSA in TBST (0.1%). Blots were washed three times in TBST (0.1%) for 5 minutes at room temperature on the rocker. Signal was developed with Femto Maximum Sensitivity substrate for 4 minutes and blots read on ChemiDoc™.

### In-Cell Western Screen of BRM Degradation in SW1573 Cells

To assess BRM degradation (Dₘₐₓ and DC₅₀), cells were seeded at 8000/well in 96-well black/clear-bottom plates in 180 μL DMEM growth media (containing 1% pen-strep, 1% HEPES and 10% FBS) per well. Plates were incubated overnight to allow adhesion. The next morning cells were treated by adding 20 μL of 10× compound (1% DMSO) to appropriate wells and returned to incubator for overnight (18-20 hours). The final DMSO concentration was 0.1%.

For processing, the plates were removed from incubator, the media was removed, and immediately 200 μL of cold (4° C.) DPBS was added to all wells. DPBS was then removed, and 50 μL of 4% paraformaldehyde (PFA) in DPBS (4° C.) was added to all wells, and the plates were incubated at room temperature for 20 minutes. PFA was then removed, and 200 μL of TBS-T containing 0.5% Triton X-100 was added to all wells, and the plates were incubated at room temperature for 30 minutes. TBS-T containing 0.5% Triton X-100 was then removed, and 50 μL of Li-Cor blocking solution was added, and the plates were incubated at room temperature for a minimum of one hour. The blocking solution was removed, and 50 μL of Li-Cor blocking solution containing primary antibody cocktail was added (1:1000 for BRM (Cell Signaling Tech. cat #11966) and 1:2000 for alpha-tubulin (Sigma cat #T6074), a control protein. Plates were then placed in cold room until the next day.

The next day plates were washed three times with TBS-T, 200 μL per well. Fifty (50) L of secondary antibody cocktail in LI-COR blocking solution (anti-rabbit_800 nm and anti-mouse_680 nm) was added to all wells (diluted 1:5000). The plates were incubated at room temperature for at least one hour while protected from light. The plates were washed twice with TBS-T, 200 μL per well.

To read each plate TBS-T was removed, and each plate was inverted tapped on a paper towel. The plates were read on the LI-COR Odyssey with default intensity settings of 5.0 for both channels. LI-COR images were analyzed with the in-cell Western feature of Image Studio Lite.

The following compounds demonstrated target protein degradation when tested under the conditions described above:

TABLE 1
Exemplary bifunctional degradation compounds of the present disclosure

| Ex. | Structure | Compound Name |
|---|---|---|
| 1 | 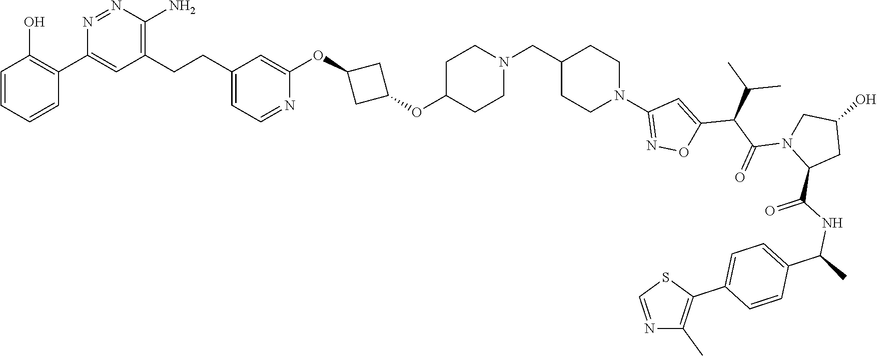 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Oc6cc(CCc7cc(-c8ccccc8O)nnc7N)ccn6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- [(4-{2-[3-amino-6-(2- hydroxyphenyl)pyridazin-4- yl]ethyl}pyridin-2- yl)oxy]cyclobutoxy]piperidin-1- yl}methyl)piperidin-1-yl]-1,2-oxazol-5- yl}butanoyl]pyrrolidine-2-carboxamide |
| 2 | 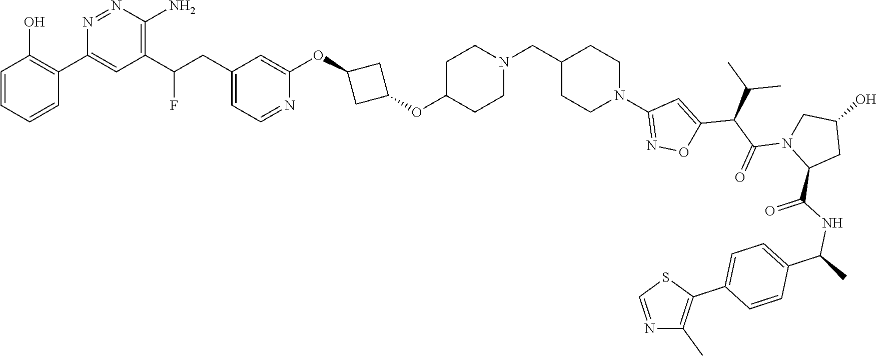 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Oc6cc(CC(F)c7cc(-c8ccccc8O)nnc7N)ccn6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- [(4-{2-[3-amino-6-(2- hydroxyphenyl)pyridazin-4-yl]-2- fluoroethyl}pyridin-2- yl)oxy]cyclobutoxy]piperidin-1- yl}methyl)piperidin-1-yl]-1,2-oxazol-5- yl}butanoyl]pyrrolidine-2-carboxamide |
| 3 | 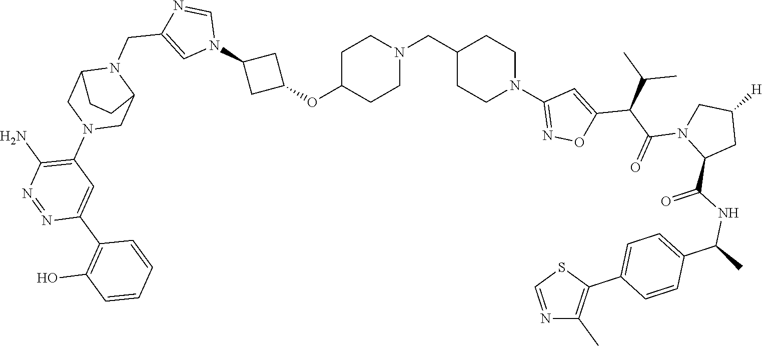 | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- [4-({3-[3-amino-6-(2- hydroxyphenyl)pyridazin-4-yl]-3,8- diazabicyclo[3.2.1]octan-8-yl}methyl)- 1H-imidazol-1-yl]cyclobutoxy]piperidin- 1-yl}methyl)piperidin-1-yl]-1,2-oxazol- 5-yl}butanoyl]pyrrolidine-2- carboxamide |
| 4 | 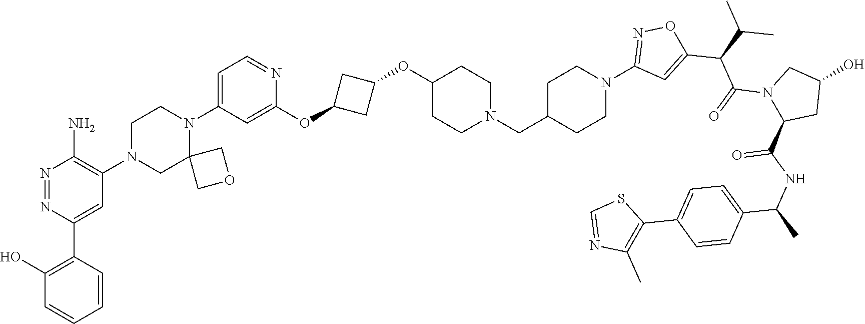 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Oc6cc(N7CCN(c8cc(-c9ccccc9O)nnc8N)CC78COC8)ccn6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- [(4-{8-[3-amino-6-(2- hydroxyphenyl)pyridazin-4-yl]-2-oxa- 5,8-diazaspiro[3.5]nonan-5-yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 5 | 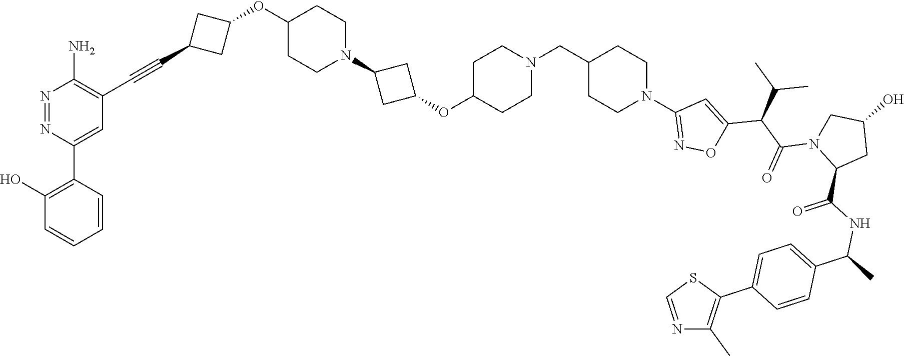 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(O[C@H]7C[C@H](C#Cc8cc(-c9ccccc9O)nnc8N)C7)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- (4-[(1r,3r)-3-{2-[3-amino-6-(2- hydroxyphenyl)pyridazin-4- yl]ethynyl}cyclobutoxy]piperidin-1- yl}cyclobutoxy]piperidin-1- yl}methyl)piperidin-1-yl]-1,2-oxazol-5- yl}butanoyl]pyrrolidine-2-carboxamide |
| 6 | 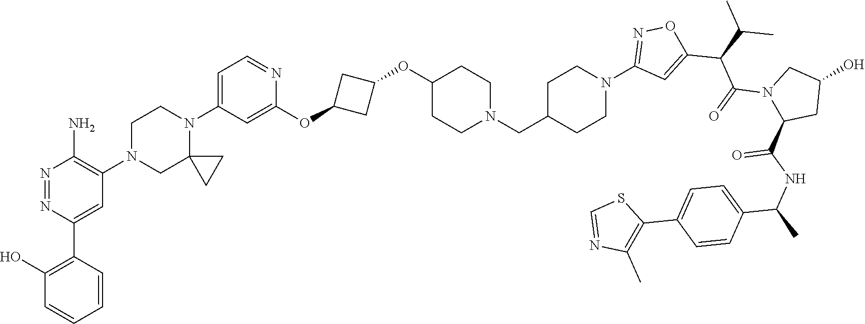 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Oc6cc(N7CCN(c8cc(-c9ccccc9O)nnc8N)CC78CC8)ccn6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- [(4-{7-[3-amino-6-(2- hydroxyphenyl)pyridazin-4-yl]-4,7- diazaspiro[2.5]octan-4-yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 7 | 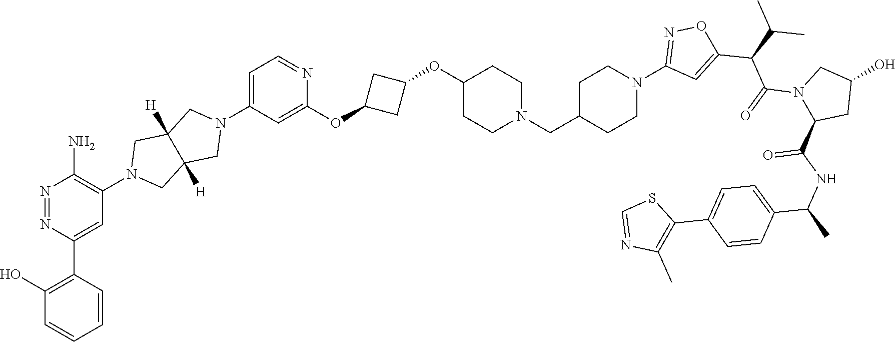 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Oc6cc(N7C[C@@H]8CN(c9cc(-c%10ccccc%10O)nnc9N)C[C@@H]8C7)ccn6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- ({4-[(3aR,6aS)-5-[3-amino-6-(2- hydroxyphenyl)pyridazin-4-yl]- octahydropyrrolo[3,4-c]pyrrol-2- yl]pyridin-2- yl}oxy)cyclobutoxy]piperidin-1- yl}methyl)piperidin-1-yl]-1,2-oxazol-5- yl}butanoyl]pyrrolidine-2-carboxamide |
| 8 | 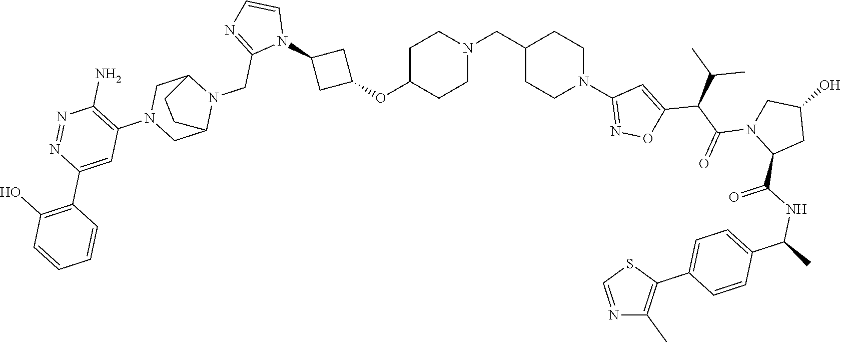 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](n6ccnc6CN6C7CCC6CN(c6cc(-c8ccccc8O)nnc6N)C7)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- [2-({3-[3-amino-6-(2- hydroxyphenyl)pyridazin-4-yl]-3,8- diazabicyclo[3.2.1]octan-8-yl}methyl)- 1H-imidazol-1-yl]cyclobutoxy]piperidin- 1-yl}methyl)piperidin-1-yl]-1,2-oxazol- 5-yl}butanoyl]pyrrolidine-2- carboxamide |
| 9 | 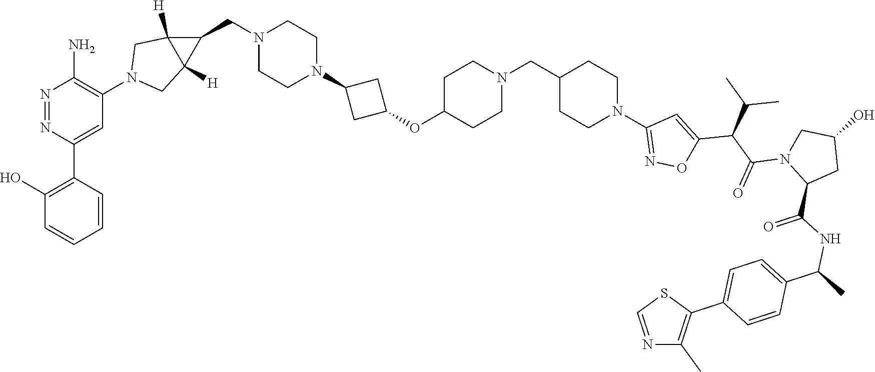 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCN(C[C@@H]7[C@H]8CN(c9cc(-c%10ccccc%10O)nnc9N)C[C@@H]78)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- (4-{[(1R,5S,6S)-3-[3-amino-6-(2- hydroxyphenyl)pyridazin-4-yl]-3- azabicyclo[3.1.0]hexan-6- yl]methyl}piperazin-1- yl)cyclobutoxy]piperidin-1- yl}methyl)piperidin-1-yl]-1,2-oxazol-5- yl}butanoyl]pyrrolidine-2-carboxamide |
| 10 | 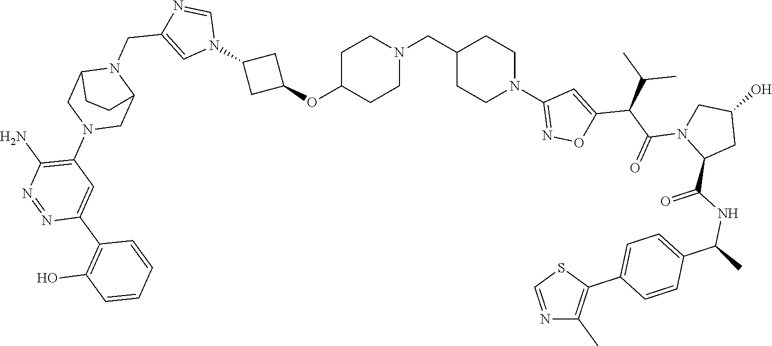 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](n6cnc(CN7C8CCC7CN(c7cc(-c9ccccc9O)nnc7N)C8)c6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R*)-3-methyl-2-{3-[4-({4-[(1r,3r)-3- [4-({3-[3-amino-6-(2- hydroxyphenyl)pyridazin-4-yl]-3,8- diazabicyclo[3.2.1]octan-8-yl}methyl)- 1H-imidazol-1-yl]cyclobutoxy]piperidin- 1-yl}methyl)piperidin-1-yl]-1,2-oxazol- 5-yl}butanoyl]pyrrolidine-2- carboxamide |
| 11 | 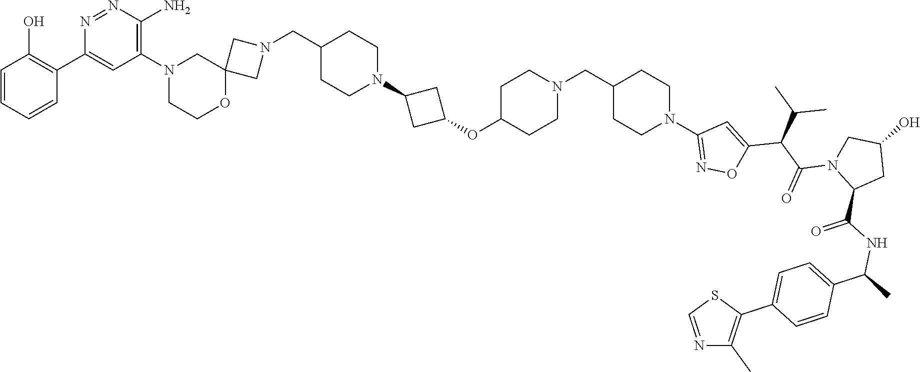 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(CN7CC8(C7)CN(c7cc(-c9ccccc9O)nnc7N)CCO8)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- [4-({ 8-[3-amino-6-(2- hydroxyphenyl)pyridazin-4-yl]-5-oxa- 2,8-diazaspiro[3.5]nonan-2- yl}methyl)piperidin-1- yl]cyclobutoxy]piperidin-1- yl}methyl)piperidin-1-yl]-1,2-oxazol-5- yl}butanoyl]pyrrolidine-2-carboxamide |
| 12 | 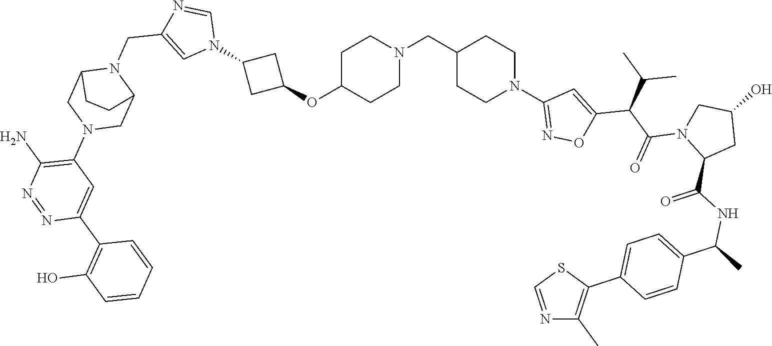 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](n6cnc(CN7C8CCC7CN(c7cc(-c9ccccc9O)nnc7N)C8)c6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- [4-({3-[3-amino-6-(2- hydroxyphenyl)pyridazin-4-yl]-3,8- diazabicyclo[3.2.1]octan-8-yl}methyl)- 1H-pyrazol-1-yl]cyclobutoxy]piperidin- 1-yl}methyl)piperidin-1-yl]-1,2-oxazol- 5-yl}butanoyl]pyrrolidine-2- carboxamide |
| 13 | 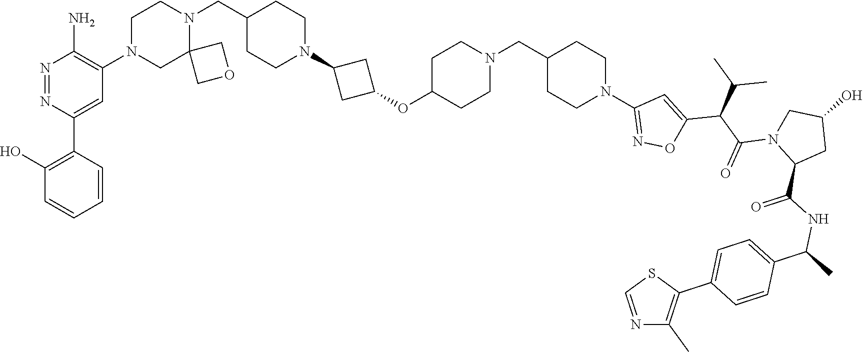 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(CN7CCN(c8cc(-c9ccccc9O)nnc8N)CC78COC8)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- [4-({ 8-[3-amino-6-(2- hydroxyphenyl)pyridazin-4-yl]-2-oxa- 5,8-diazaspiro[3.5]nonan-5- yl}methyl)piperidin-1- yl]cyclobutoxy]piperidin-1- yl}methyl)piperidin-1-yl]-1,2-oxazol-5- yl}butanoyl]pyrrolidine-2-carboxamide |
| 14 | 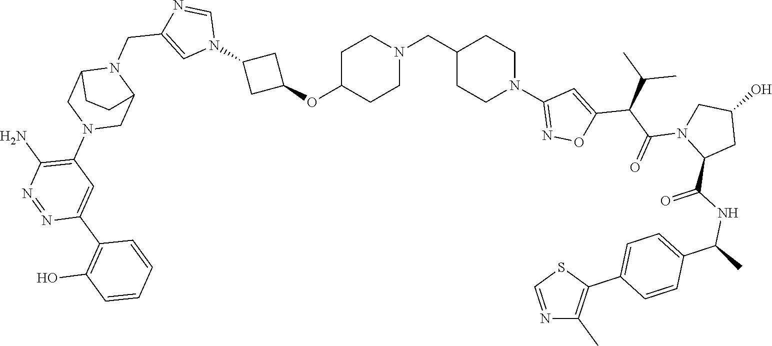 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](n6cnc(CN7C8CCC7CN(c7cc(-c9ccccc9O)nnc7N)C8)c6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- [3-({3-[3-amino-6-(2- hydroxyphenyl)pyridazin-4-yl]-3,8- diazabicyclo[3.2.1]octan-8-yl}methyl)- 1H-pyrazol-1-yl]cyclobutoxy]piperidin- 1-yl}methyl)piperidin-1-yl]-1,2-oxazol- 5-yl}butanoyl]pyrrolidine-2- carboxamide |
| 15 | 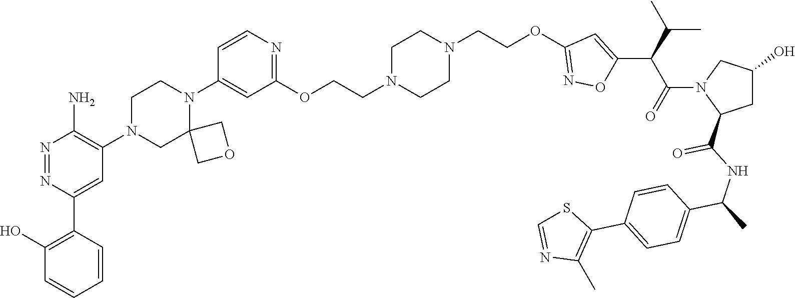 [SMILES: Cc1ncsc1-c1ccc([C@H](C)NC(=O)[C@@H]2C[C@@H](O)CN2C(=O)[C@@H](c2cc(OCCN3CCN(CCOc4cc(N5CCN(c6cc(-c7ccccc7O)nnc6N)CC56COC6)ccn4)CC3)no2)C(C)C)cc1] | (2S,4R)-1-[(2R)-2-{3-[2-(4-{2-[(4-{8-[3- amino-6-(2-hydroxyphenyl)pyridazin-4- yl]-2-oxa-5,8-diazaspiro[3.5]nonan-5- yl}pyridin-2-yl)oxy]ethyl}piperazin-1- yl)ethoxy]-1,2-oxazol-5-yl}-3- methylbutanoyl]-4-hydroxy-N-[(1S)-1- [4-(4-methyl-1,3-thiazol-5- yl)phenyl]ethyl]pyrrolidine-2- carboxamide |
| 16 | 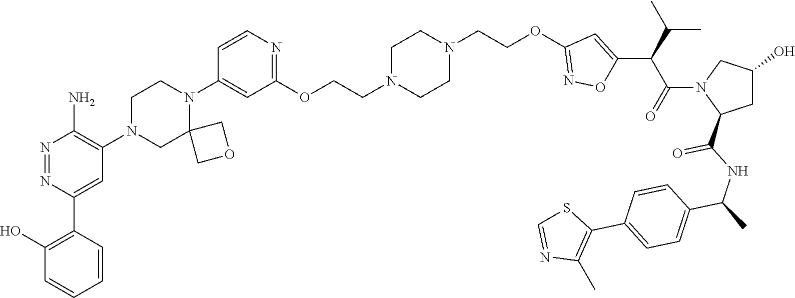 [SMILES: Cc1ncsc1-c1ccc([C@H](C)NC(=O)[C@@H]2C[C@@H](O)CN2C(=O)[C@@H](c2cc(OCCN3CCN(CCOc4cc(N5CCN(c6cc(-c7ccccc7O)nnc6N)CC56COC6)ccn4)CC3)no2)C(C)C)cc1] | (2S,4R)-1-[(2S)-2-{3-[2-(4-{2-[(4-{8-[3- amino-6-(2-hydroxyphenyl)pyridazin-4- yl]-2-oxa-5,8-diazaspiro[3.5]nonan-5- yl}pyridin-2-yl)oxy]ethyl}piperazin-1- yl)ethoxy]-1,2-oxazol-5-yl}-3- methylbutanoyl]-4-hydroxy-N-[(1S)-1- [6-(4-methyl-1,3-thiazol-5-yl)pyridin-3- yl]ethyl]pyrrolidine-2-carboxamide |
| 17 | 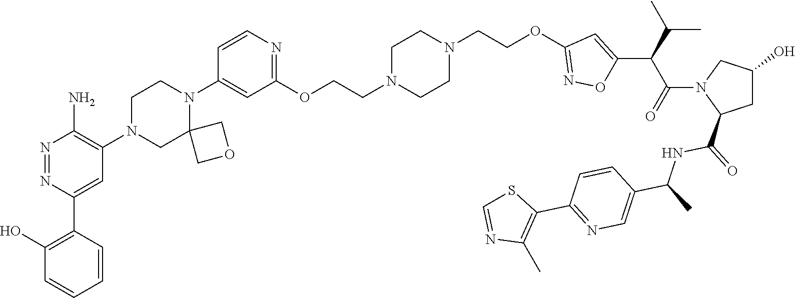 [SMILES: Cc1ncsc1-c1ccc([C@H](C)NC(=O)[C@@H]2C[C@@H](O)CN2C(=O)[C@@H](c2cc(OCCN3CCN(CCOc4cc(N5CCN(c6cc(-c7ccccc7O)nnc6N)CC56COC6)ccn4)CC3)no2)C(C)C)cn1] | (2S,4R)-1-[(2R)-2-{3-[2-(4-{2-[(4-{8-[3- amino-6-(2-hydroxyphenyl)pyridazin-4- yl]-2-oxa-5,8-diazaspiro[3.5]nonan-5- yl}pyridin-2-yl)oxy]ethyl}piperazin-1- yl)ethoxy]-1,2-oxazol-5-yl}-3- methylbutanoyl]-4-hydroxy-N-[(1S)-1- [6-(4-methyl-1,3-thiazol-5-yl)pyridin-3- yl]ethyl]pyrrolidine-2-carboxamide |
| 18 | 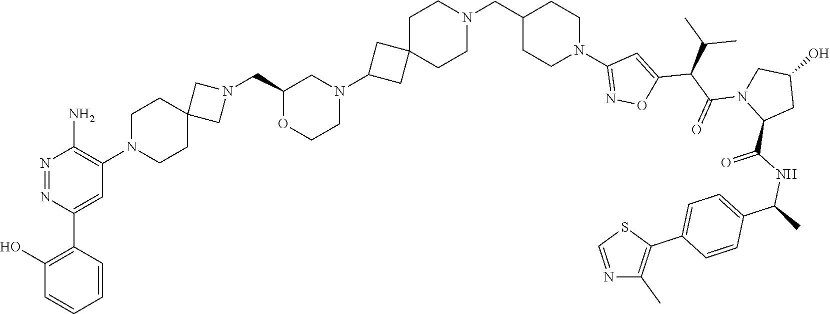 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](CN6CC7(CCN(c8cc(-c9ccccc9O)nnc8N)CC7)C6)C4)C5)CC3)no2)C(C)C)cc1] | (2S,4R)-1-[(2R)-2-{3-[4-({2-[(2S)-2- ({7-[3-amino-6-(2- hydroxyphenyl)pyridazin-4-yl]-2,7- diazaspiro[3.5]nonan-2- yl}methyl)morpholin-4-yl]-7- azaspiro[3.5]nonan-7- yl}methyl)piperidin-1-yl]-1,2-oxazol-5- yl}-3-methylbutanoyl]-4-hydroxy-N- [(1S)-1-[4-(4-methyl-1,3-thiazol-5- yl)phenyl]ethyl]pyrrolidine-2- carboxamide |
| 19 | 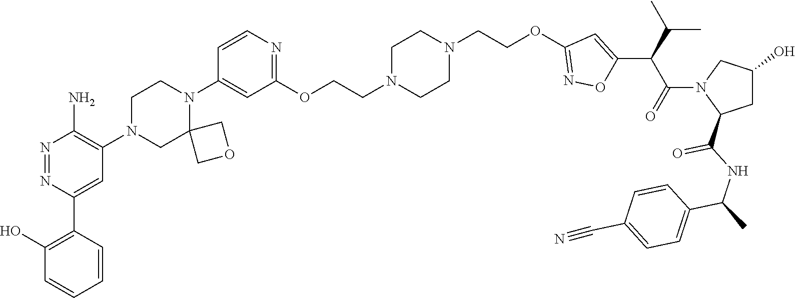 [SMILES: CC(C)[C@@H](C(=O)N1C[C@H](O)C[C@H]1C(=O)N[C@@H](C)c1ccc(C#N)cc1)c1cc(OCCN2CCN(CCOc3cc(N4CCN(c5cc(-c6ccccc6O)nnc5N)CC45COC5)ccn3)CC2)no1] | (2S,4R)-1-[(2R)-2-{3-[2-(4-{2-[(4-{8-[3- amino-6-(2-hydroxyphenyl)pyridazin-4- yl]-2-oxa-5,8-diazaspiro[3.5]nonan-5- yl}pyridin-2-yl)oxy]ethyl}piperazin-1- yl)ethoxy]-1,2-oxazol-5-yl}-3- methylbutanoyl]-N-[(1S)-1-(4- cyanophenyl)ethyl]-4- hydroxypyrrolidine-2-carboxamide |
| 20 | 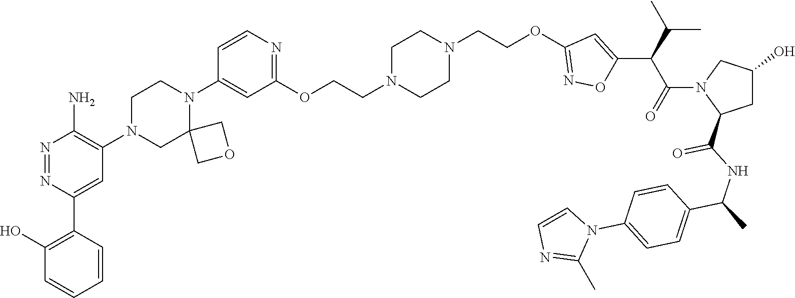 [SMILES: Cc1nccn1-c1ccc([C@H](C)NC(=O)[C@@H]2C[C@@H](O)CN2C(=O)[C@@H](c2cc(OCCN3CCN(CCOc4cc(N5CCN(c6cc(-c7ccccc7O)nnc6N)CC56COC6)ccn4)CC3)no2)C(C)C)cc1] | (2S,4R)-1-[(2R)-2-{3-[2-(4-{2-[(4-{8-[3- amino-6-(2-hydroxyphenyl)pyridazin-4- yl]-2-oxa-5,8-diazaspiro[3.5]nonan-5- yl}pyridin-2-yl)oxy]ethyl}piperazin-1- yl)ethoxy]-1,2-oxazol-5-yl}-3- methylbutanoyl]-4-hydroxy-N-[(1S)-1- [4-(2-methyl-1H-imidazol-1- yl)phenyl]ethyl]pyrrolidine-2- carboxamide |
| 21 | 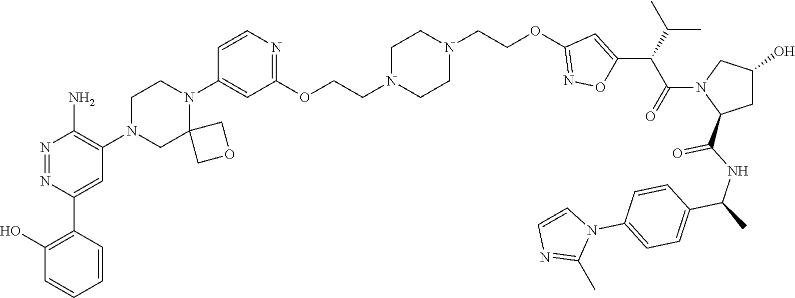 [SMILES: Cc1nccn1-c1ccc([C@H](C)NC(=O)[C@@H]2C[C@@H](O)CN2C(=O)[C@H](c2cc(OCCN3CCN(CCOc4cc(N5CCN(c6cc(-c7ccccc7O)nnc6N)CC56COC6)ccn4)CC3)no2)C(C)C)cc1] | (2S,4R)-1-[(2S)-2-{3-[2-(4-{2-[(4-{8-[3- amino-6-(2-hydroxyphenyl)pyridazin-4- yl]-2-oxa-5,8-diazaspiro[3.5]nonan-5- yl}pyridin-2-yl)oxy]ethyl}piperazin-1- yl)ethoxy]-1,2-oxazol-5-yl}-3- methylbutanoyl]-4-hydroxy-N-[(1S)-1- [4-(2-methyl-1H-imidazol-1- yl)phenyl]ethyl]pyrrolidine-2- carboxamide |
| 22 | 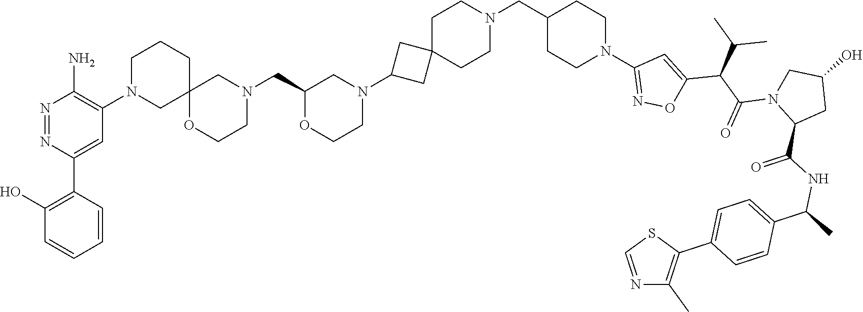 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](CN6CCOC7(CCCN(c8cc(-c9ccccc9O)nnc8N)C7)C6)C4)C5)CC3)no2)C(C)C)cc1] | (2S,4R)-1-[(2R)-2-{3-[4-({2-[(2S)-2- ({8-[3-amino-6-(2- hydroxyphenyl)pyridazin-4-yl]-1-oxa- 4,8-diazaspiro[5.5]undecan-4- yl}methyl)morpholin-4-yl]-7- azaspiro[3.5]nonan-7- yl}methyl)piperidin-1-yl]-1,2-oxazol-5- yl}-3-methylbutanoyl]-4-hydroxy-N- [(1S)-1-[4-(4-methyl-1,3-thiazol-5- yl)phenyl]ethyl]pyrrolidine-2- carboxamide |
| 23 | 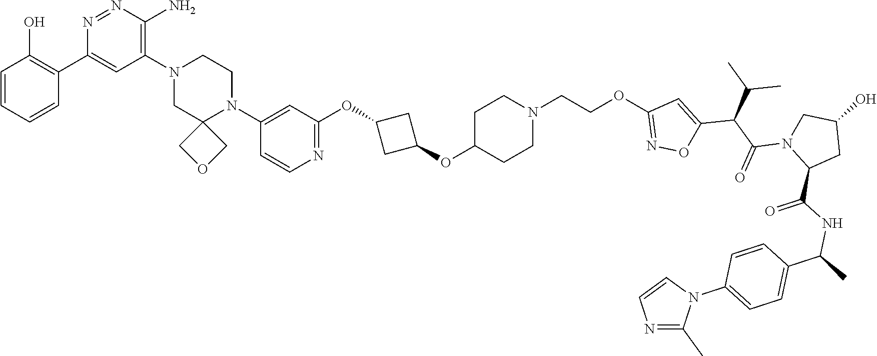 [SMILES: Cc1nccn1-c1ccc([C@H](C)NC(=O)[C@@H]2C[C@@H](O)CN2C(=O)[C@@H](c2cc(OCCN3CCC(O[C@H]4C[C@H](Oc5cc(N6CCN(c7cc(-c8ccccc8O)nnc7N)CC67COC7)ccn5)C4)CC3)no2)C(C)C)cc1] | (2S,4R)-4-hydroxy-N-[(1S)-1-[4-(2- methyl-1H-imidazol-1-yl)phenyl]ethyl]- 1-[(2R)-3-methyl-2-[3-(2-{4-[(1r,3r)-3- [(4-{8-[3-amino-6-(2- hydroxyphenyl)pyridazin-4-yl]-2-oxa- 5,8-diazaspiro[3.5]nonan-5-yl}pyridin-2- yl)oxy]cyclobutoxy]piperidin-1- yl}ethoxy)-1,2-oxazol-5- yl]butanoyl]pyrrolidine-2-carboxamide |
| 24 | 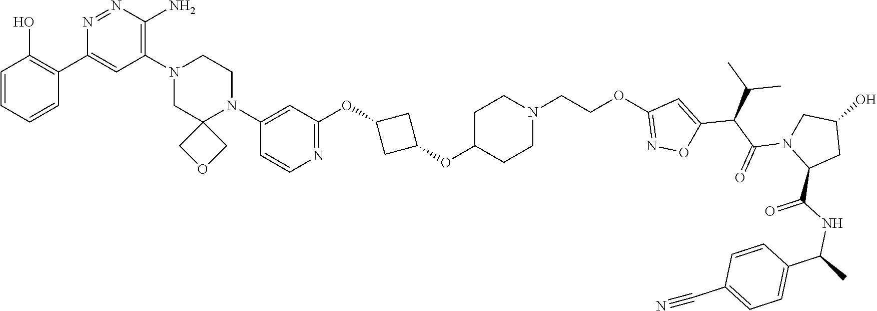 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@@H](Oc4cc(N5CCN(c6cc(-c7ccccc7O)nnc6N)CC56COC6)ccn4)C3)CC2)no1] | (2S,4R)-N-[(1S)-1-(4- cyanophenyl)ethyl]-4-hydroxy-1-[(2R)- 3-methyl-2-[3-(2-{4-[(1r,3r)-3-[(4-{8-[3- amino-6-(2-hydroxyphenyl)pyridazin-4- yl]-2-oxa-5,8-diazaspiro[3.5]nonan-5- yl}pyridin-2- yl)oxy]cyclobutoxy]piperidin-1- yl}ethoxy)-1,2-oxazol-5- yl]butanoyl]pyrrolidine-2-carboxamide |
| 25 | 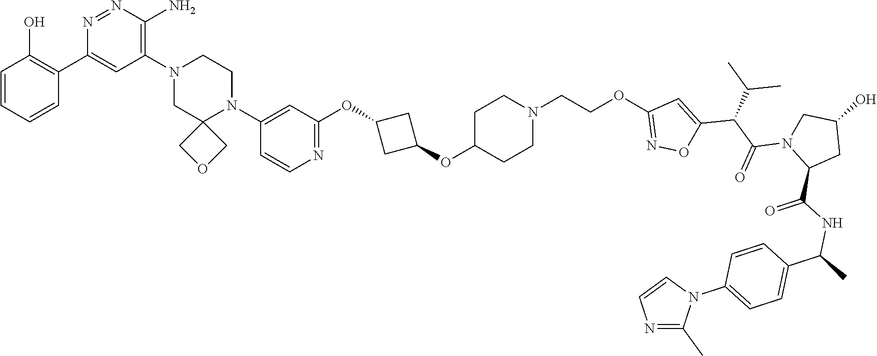 [SMILES: Cc1nccn1-c1ccc([C@H](C)NC(=O)[C@@H]2C[C@@H](O)CN2C(=O)[C@H](c2cc(OCCN3CCC(O[C@H]4C[C@H](Oc5cc(N6CCN(c7cc(-c8ccccc8O)nnc7N)CC67COC7)ccn5)C4)CC3)no2)C(C)C)cc1] | (2S,4R)-4-hydroxy-N-[(1S)-1-[4-(2- methyl-1H-imidazol-1-yl)phenyl]ethyl]- 1-[(2S)-3-methyl-2-[3-(2-{4-[(1r,3r)-3- [(4-{8-[3-amino-6-(2- hydroxyphenyl)pyridazin-4-yl]-2-oxa- 5,8-diazaspiro[3.5]nonan-5-yl}pyridin-2- yl)oxy]cyclobutoxy]piperidin-1- yl}ethoxy)-1,2-oxazol-5- yl]butanoyl]pyrrolidine-2-carboxamide |
| 26 | 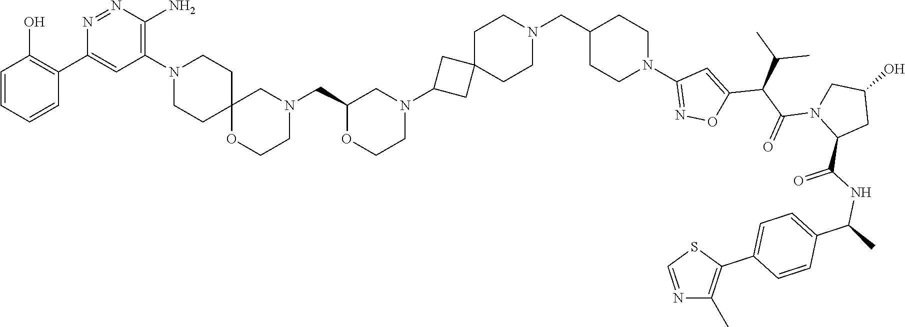 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](CN6CCOC7(CCN(c8cc(-c9ccccc9O)nnc8N)CC7)C6)C4)C5)CC3)no2)C(C)C)cc1] | (2S,4R)-1-[(2R)-2-{3-[4-({2-[(2S)-2- ({9-[3-amino-6-(2- hydroxyphenyl)pyridazin-4-yl]-1-oxa- 4,9-diazaspiro[5.5]undecan-4- yl}methyl)morpholin-4-yl]-7- azaspiro[3.5]nonan-7- yl}methyl)piperidin-1-yl]-1,2-oxazol-5- yl}-3-methylbutanoyl]-4-hydroxy-N- [(1S)-1-[4-(4-methyl-1,3-thiazol-5- yl)phenyl]ethyl]pyrrolidine-2- carboxamide |
| 27 | 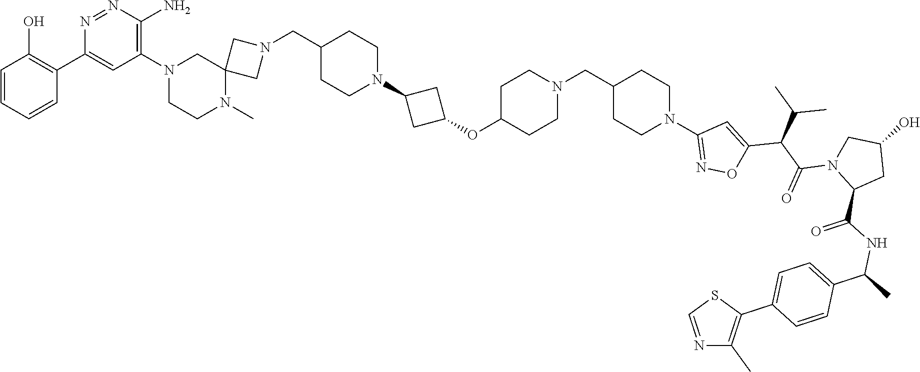 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(CN7CC8(C7)CN(c7cc(-c9ccccc9O)nnc7N)CCN8C)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- [4-({ 8-[3-amino-6-(2- hydroxyphenyl)pyridazin-4-yl]-5- methyl-2,5,8-triazaspiro[3.5]nonan-2- yl}methyl)piperidin-1- yl]cyclobutoxy]piperidin-1- yl}methyl)piperidin-1-yl]-1,2-oxazol-5- yl}butanoyl]pyrrolidine-2-carboxamide |
| 28 | 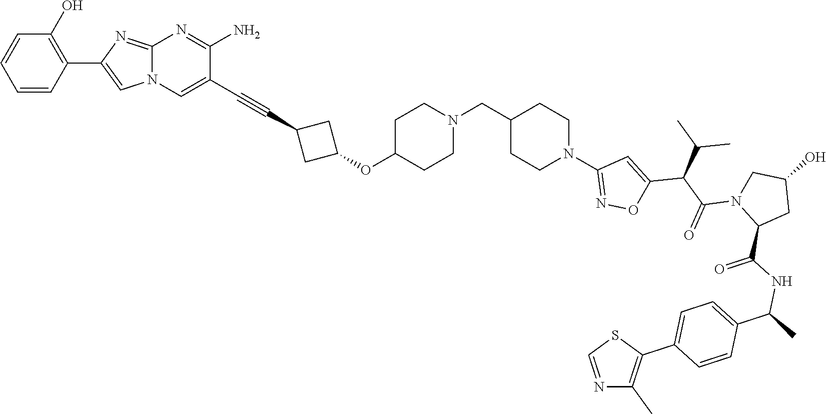 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](C#Cc6cn7cc(-c8ccccc8O)nc7nc6N)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- (2-[7-amino-2-(2- hydroxyphenyl)imidazo[1,2-a]pyrimidin- 6-yl]ethynyl}cyclobutoxy]piperidin-1- yl}methyl)piperidin-1-yl]-1,2-oxazol-5- yl}butanoyl]pyrrolidine-2-carboxamide |
| 29 | 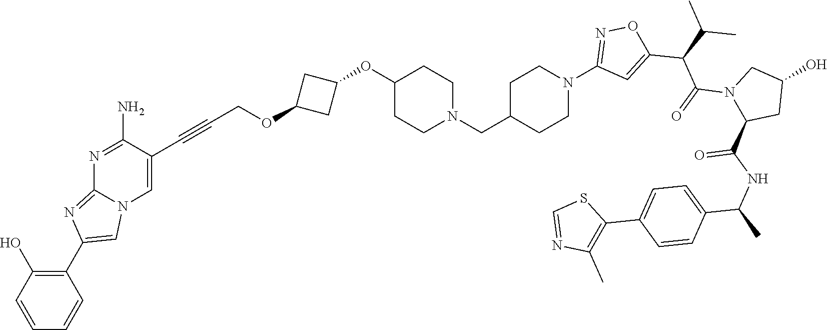 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](OCC#Cc6cn7cc(-c8ccccc8O)nc7nc6N)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- ({3-[7-amino-2-(2- hydroxyphenyl)imidazo[1,2-a]pyrimidin- 6-yl]prop-2-yn-1- yl}oxy)cyclobutoxy]piperidin-1- yl}methyl)piperidin-1-yl]-1,2-oxazol-5- yl}butanoyl]pyrrolidine-2-carboxamide |
| 30 | 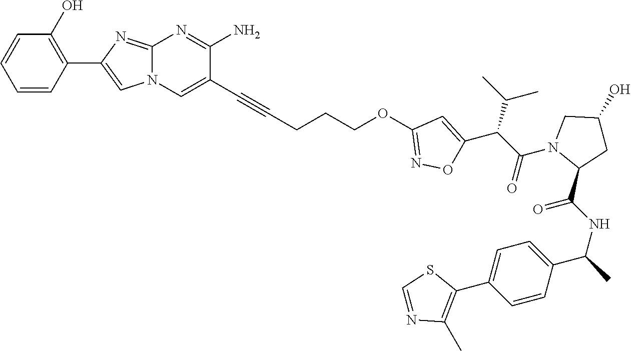 [SMILES: Cc1ncsc1-c1ccc([C@H](C)NC(=O)[C@@H]2C[C@@H](O)CN2C(=O)[C@H](c2cc(OCCCC#Cc3cn4cc(-c5ccccc5O)nc4nc3N)no2)C(C)C)cc1] | (2S,4R)-1-[(2S)-2-[3-({5-[7-amino-2-(2- hydroxyphenyl)imidazo[1,2-a]pyrimidin- 6-yl]pent-4-yn-1-yl}oxy)-1,2-oxazol-5- yl]-3-methylbutanoyl]-4-hydroxy-N- [(1S)-1-[4-(4-methyl-1,3-thiazol-5- yl)phenyl]ethyl]pyrrolidine-2- carboxamide |
| 31 | 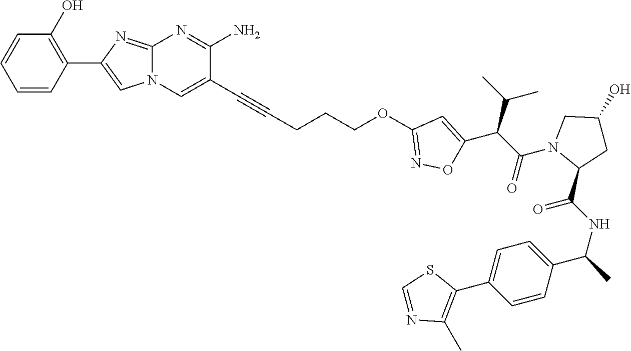 [SMILES: Cc1ncsc1-c1ccc([C@H](C)NC(=O)[C@@H]2C[C@@H](O)CN2C(=O)[C@@H](c2cc(OCCCC#Cc3cn4cc(-c5ccccc5O)nc4nc3N)no2)C(C)C)cc1] | (2S,4R)-1-[(2R)-2-[3-({5-[7-amino-2-(2- hydroxyphenyl)imidazo[1,2-a]pyrimidin- 6-yl]pent-4-yn-1-yl}oxy)-1,2-oxazol-5- yl]-3-methylbutanoyl]-4-hydroxy-N- [(1S)-1-[4-(4-methyl-1,3-thiazol-5- yl)phenyl]ethyl]pyrrolidine-2- carboxamide |
| 32 | 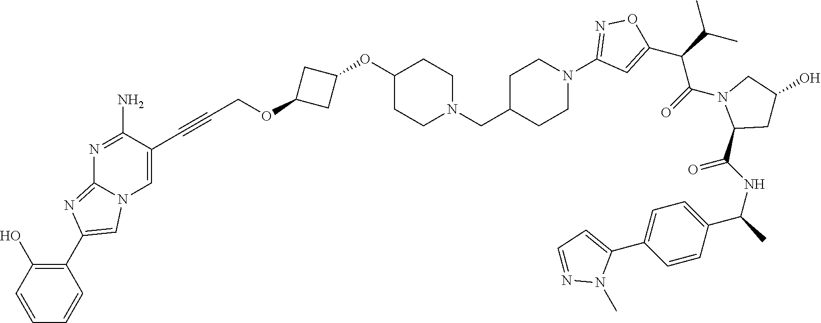 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](OCC#Cc5cn6cc(-c7ccccc7O)nc6nc5N)C4)CC3)CC2)no1] | (2S,4R)-4-hydroxy-N-[(1S)-1-[4-(1- methyl-1H-pyrazol-5-yl)phenyl]ethyl]-1- [(2R)-3-methyl-2-{3-[4-({4-[(1r,3r)-3- ({3-[7-amino-2-(2- hydroxyphenyl)imidazo[1,2-a]pyrimidin- 6-yl]prop-2-yn-1- yl}oxy)cyclobutoxy]piperidin-1- yl}methyl)piperidin-1-yl]-1,2-oxazol-5- yl}butanoyl]pyrrolidine-2-carboxamide |
| 33 | 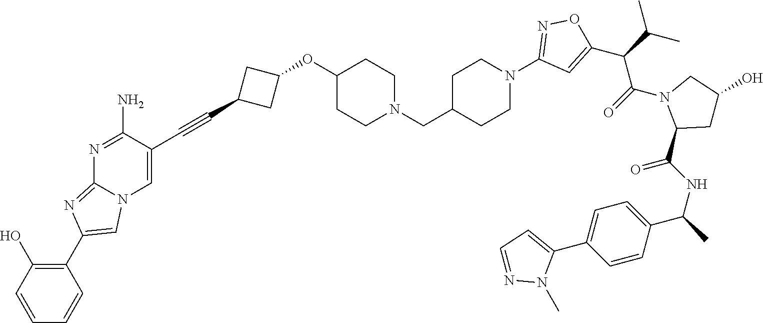 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](C#Cc5cn6cc(-c7ccccc7O)nc6nc5N)C4)CC3)CC2)no1] | (2S,4R)-4-hydroxy-N-[(1S)-1-[4-(1- methyl-1H-pyrazol-5-yl)phenyl]ethyl]-1- [(2R)-3-methyl-2-{3-[4-({4-[(1r,3r)-3- {2-[7-amino-2-(2- hydroxyphenyl)imidazo[1,2-a]pyrimidin- 6-yl]ethynyl}cyclobutoxy]piperidin-1- yl}methyl)piperidin-1-yl]-1,2-oxazol-5- yl}butanoyl]pyrrolidine-2-carboxamide |
| 34 | 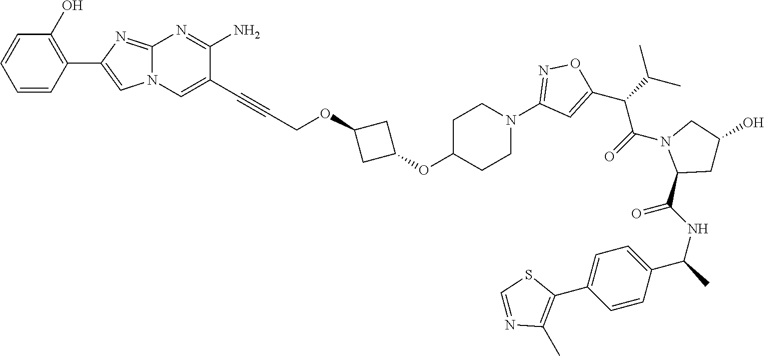 [SMILES: Cc1ncsc1-c1ccc([C@H](C)NC(=O)[C@@H]2C[C@@H](O)CN2C(=O)[C@H](c2cc(N3CCC(O[C@H]4C[C@H](OCC#Cc5cn6cc(-c7ccccc7O)nc6nc5N)C4)CC3)no2)C(C)C)cc1] | (2S,4R)-4-hydroxy-N-[(1S)-1-[4-(4- methyl-1,3-thiazol-5-yl)phenyl]ethyl]-1- [(2S)-3-methyl-2-(3-{4-[(1r,3r)-3-({3-[7- amino-2-(2-hydroxyphenyl)imidazo[1,2- a]pyrimidin-6-yl]prop-2-yn-1- yl}oxy)cyclobutoxy]piperidin-1-yl}-1,2- oxazol-5-yl)butanoyl]pyrrolidine-2- carboxamide |
| 35 | 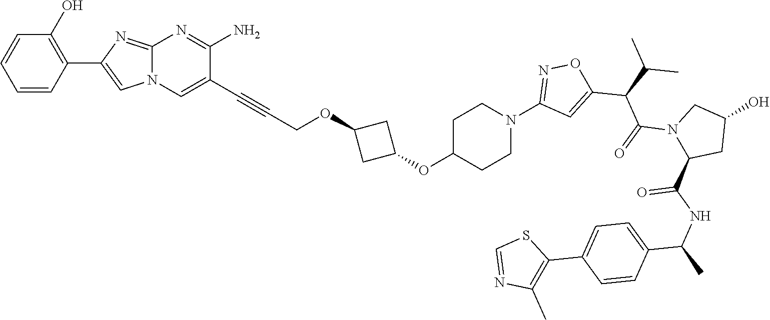 [SMILES: Cc1ncsc1-c1ccc([C@H](C)NC(=O)[C@@H]2C[C@@H](O)CN2C(=O)[C@@H](c2cc(N3CCC(O[C@H]4C[C@H](OCC#Cc5cn6cc(-c7ccccc7O)nc6nc5N)C4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-(3-{4-[(1r,3r)-3-({3- [7-amino-2-(2- hydroxyphenyl)imidazo[1,2-a]pyrimidin- 6-yl]prop-2-yn-1- yl}oxy)cyclobutoxy]piperidin-1-yl}-1,2- oxazol-5-yl)butanoyl]pyrrolidine-2- carboxamide |
| 36 | 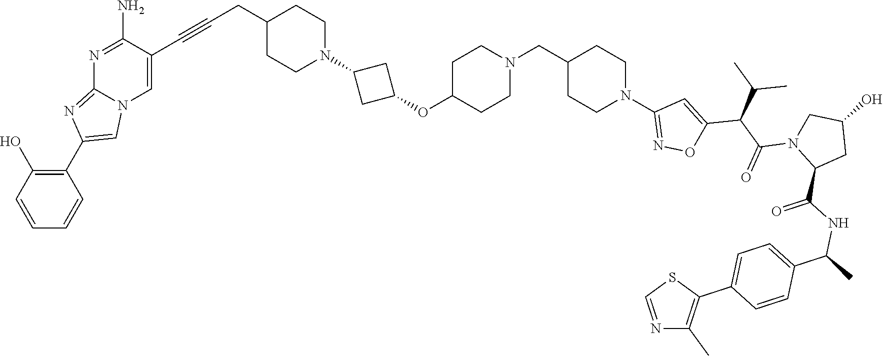 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@@H](N6CCC(CC#Cc7cn8cc(-c9ccccc9O)nc8nc7N)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1s,3s)-3- (4-{3-[7-amino-2-(2- hydroxyphenyl)imidazo[1,2-a]pyrimidin- 6-yl]prop-2-yn-1-yl}piperidin-1- yl)cyclobutoxy]piperidin-1- yl}methyl)piperidin-1-yl]-1,2-oxazol-5- yl}butanoyl]pyrrolidine-2-carboxamide |
| 37 | 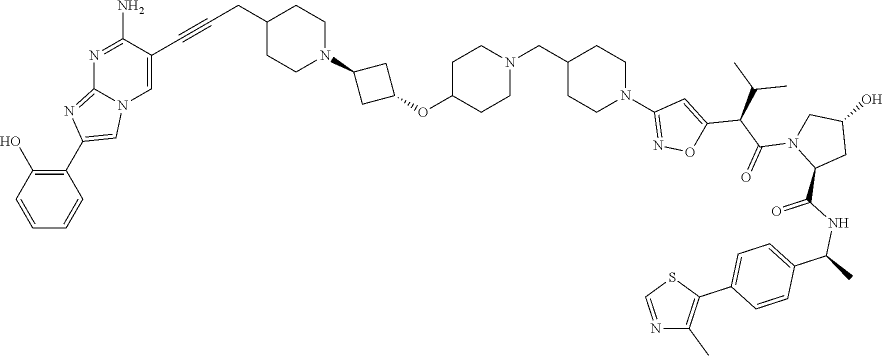 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(CC#Cc7cn8cc(-c9ccccc9O)nc8nc7N)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- (4-{3-[7-amino-2-(2- hydroxyphenyl)imidazo[1,2-a]pyrimidin- 6-yl]prop-2-yn-1-yl}piperidin-1- yl)cyclobutoxy]piperidin-1- yl}methyl)piperidin-1-yl]-1,2-oxazol-5- yl}butanoyl]pyrrolidine-2-carboxamide |
| 38 | 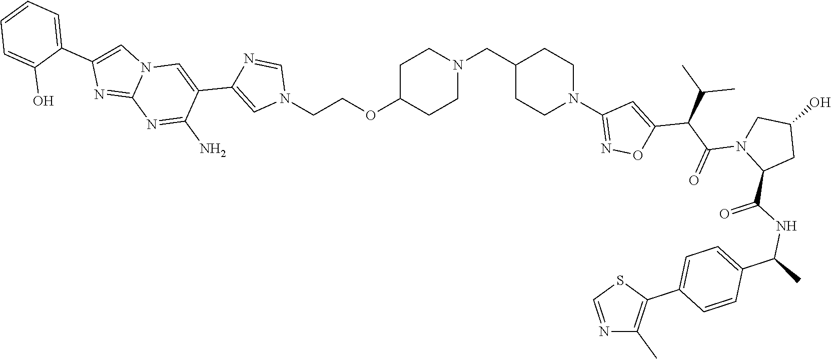 [SMILES: Cc1ncsc1-c1ccc([C@H](C)NC(=O)[C@@H]2C[C@@H](O)CN2C(=O)[C@@H](c2cc(N3CCC(CN4CCC(OCCn5cnc(-c6cn7cc(-c8ccccc8O)nc7nc6N)c5)CC4)CC3)no2)C(C)C)cc1] | (2S,4R)-1-[(2R)-2-[3-(4-{[4-(2-{4-[7- amino-2-(2-hydroxyphenyl)imidazo[1,2- a]pyrimidin-6-yl]-1H-imidazol-1- yl}ethoxy)piperidin-1- yl]methyl}piperidin-1-yl)-1,2-oxazol-5- yl]-3-methylbutanoyl]-4-hydroxy-N- [(1S)-1-[4-(4-methyl-1,3-thiazol-5- yl)phenyl]ethyl]pyrrolidine-2- carboxamide |
| 39 | 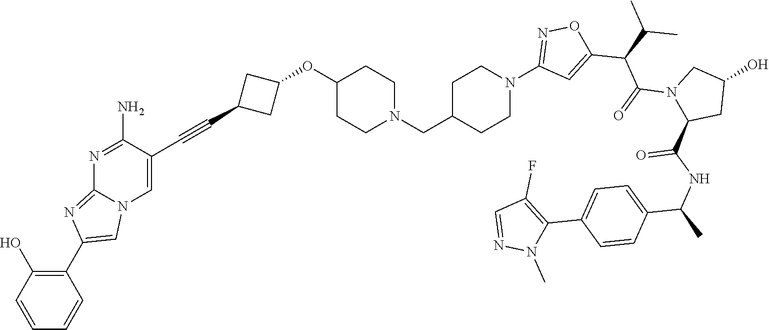 [SMILES: CC(C)[C@@H](C(=O)N1C[C@H](O)C[C@H]1C(=O)N[C@@H](C)c1ccc(-c2c(F)cnn2C)cc1)c1cc(N2CCC(CN3CCC(O[C@H]4C[C@H](C#Cc5cn6cc(-c7ccccc7O)nc6nc5N)C4)CC3)CC2)no1] | (2S,4R)-N-[(1S)-1-[4-(4-fluoro-1- methyl-1H-pyrazol-5-yl)phenyl]ethyl]-4- hydroxy-1-[(2R)-3-methyl-2-{3-[4-({4- [(1r,3r)-3-{2-[7-amino-2-(2- hydroxyphenyl)imidazo[1,2-a]pyrimidin- 6-yl]ethynyl}cyclobutoxy]piperidin-1- yl}methyl)piperidin-1-yl]-1,2-oxazol-5- yl}butanoyl]pyrrolidine-2-carboxamide |
| 40 | 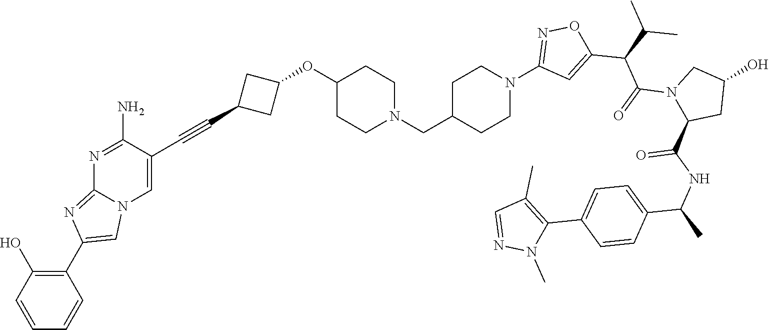 [SMILES: Cc1cnn(C)c1-c1ccc([C@H](C)NC(=O)[C@@H]2C[C@@H](O)CN2C(=O)[C@@H](c2cc(N3CCC(CN4CCC(O[C@H]5C[C@H](C#Cc6cn7cc(-c8ccccc8O)nc7nc6N)C5)CC4)CC3)no2)C(C)C)cc1] | (2S,4R)-N-[(1S)-1-[4-(1,4-dimethyl-1H- pyrazol-5-yl)phenyl]ethyl]-4-hydroxy-1- [(2R)-3-methyl-2-{3-[4-({4-[(1r,3r)-3- {2-[7-amino-2-(2- hydroxyphenyl)imidazo[1,2-a]pyrimidin- 6-yl]ethynyl}cyclobutoxy]piperidin-1- yl}methyl)piperidin-1-yl]-1,2-oxazol-5- yl}butanoyl]pyrrolidine-2-carboxamide |
| 41 | 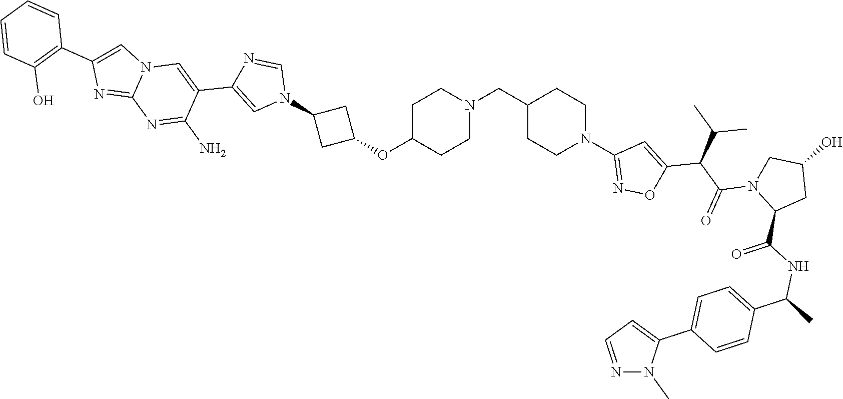 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](n5cnc(-c6cn7cc(-c8ccccc8O)nc7nc6N)c5)C4)CC3)CC2)no1] | (2S,4R)-4-hydroxy-N-[(1S)-1-[4-(1- methyl-1H-pyrazol-5-yl)phenyl]ethyl]-1- [(2R)-3-methyl-2-{3-[4-({4-[(1r,3r)-3- {4-[7-amino-2-(2- hydroxyphenyl)imidazo[1,2-a]pyrimidin- 6-yl]-1H-pyrazol-1- yl}cyclobutoxy]piperidin-1- yl}methyl)piperidin-1-yl]-1,2-oxazol-5- yl}butanoyl]pyrrolidine-2-carboxamide |
| 42 | 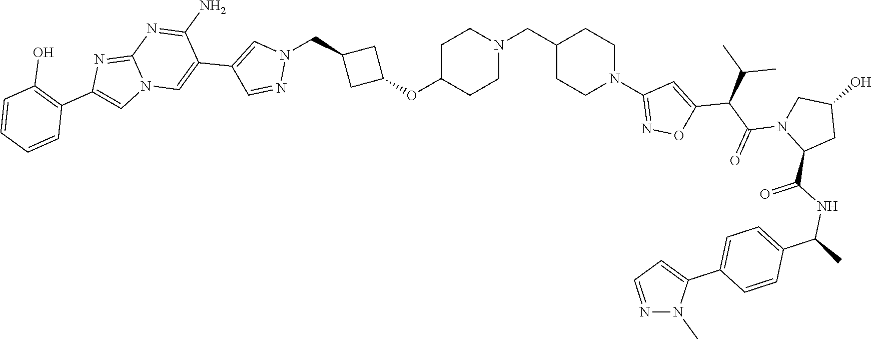 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](Cn5cc(-c6cn7cc(-c8ccccc8O)nc7nc6N)cn5)C4)CC3)CC2)no1] | (2S,4R)-4-hydroxy-N-[(1S)-1-[4-(1- methyl-1H-pyrazol-5-yl)phenyl]ethyl]-1- [(2R)-3-methyl-2-{3-[4-({4-[(1r,3r)-3- ({4-[7-amino-2-(2- hydroxyphenyl)imidazo[1,2-a]pyrimidin- 6-yl]-1H-pyrazol-1- yl}methyl)cyclobutoxy]piperidin-1- yl}methyl)piperidin-1-yl]-1,2-oxazol-5- yl}butanoyl]pyrrolidine-2-carboxamide |
| 43 | 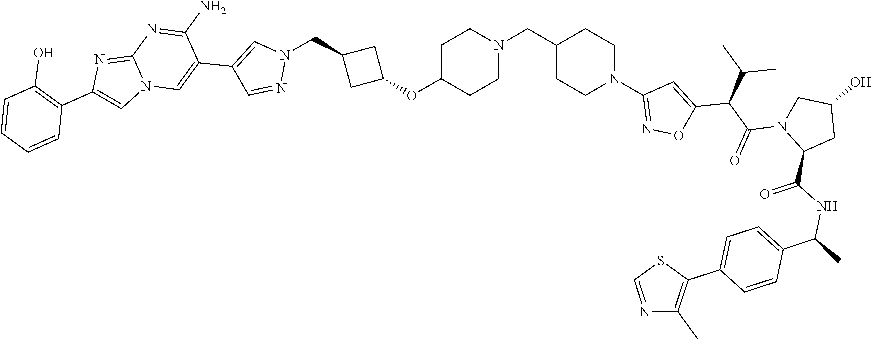 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Cn6cc(-c7cn8cc(-c9ccccc9O)nc8nc7N)cn6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- ({4-[7-amino-2-(2- hydroxyphenyl)imidazo[1,2-a]pyrimidin- 6-yl]-1H-imidazol-1- yl}methyl)cyclobutoxy]piperidin-1- yl}methyl)piperidin-1-yl]-1,2-oxazol-5- yl}butanoyl]pyrrolidine-2-carboxamide |
| 44 | 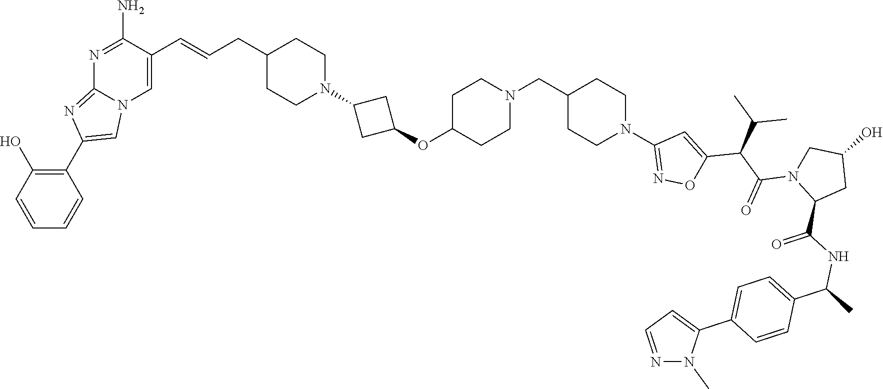 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](N5CCC(C/C=C/c6cn7cc(-c8ccccc8O)nc7nc6N)CC5)C4)CC3)CC2)no1] | (2S,4R)-4-hydroxy-N-[(1S)-1-[4-(1- methyl-1H-pyrazol-5-yl)phenyl]ethyl]-1- [(2R)-3-methyl-2-{3-[4-({4-[(1r,3r)-3- {4-[(2E)-3-[7-amino-2-(2- hydroxyphenyl)imidazo[1,2-a]pyrimidin- 6-yl]prop-2-en-1-yl]piperidin-1- yl}cyclobutoxy]piperidin-1- yl}methyl)piperidin-1-yl]-1,2-oxazol-5- yl}butanoyl]pyrrolidine-2-carboxamide |
| 45 | 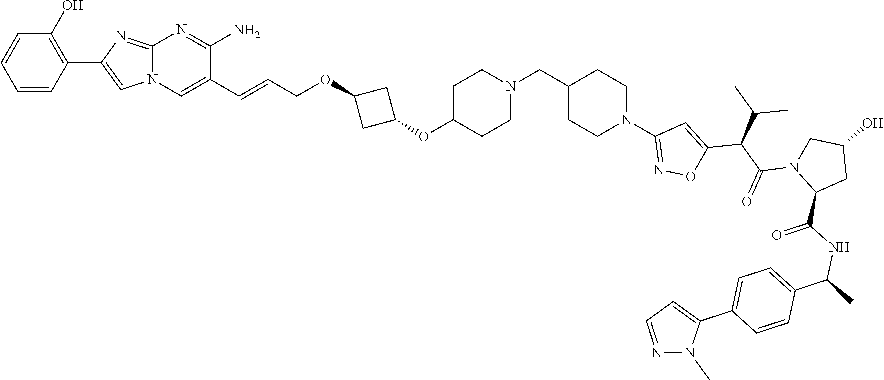 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](OC/C=C/c5cn6cc(-c7ccccc7O)nc6nc5N)C4)CC3)CC2)no1] | (2S,4R)-4-hydroxy-N-[(1S)-1-[4-(1- methyl-1H-pyrazol-5-yl)phenyl]ethyl]-1- [(2R)-3-methyl-2-{3-[4-({4-[(1r,3r)-3- {[(2E)-3-[7-amino-2-(2- hydroxyphenyl)imidazo[1,2-a]pyrimidin- 6-yl]prop-2-en-1- yl]oxy}cyclobutoxy]piperidin-1- yl}methyl)piperidin-1-yl]-1,2-oxazol-5- yl}butanoyl]pyrrolidine-2-carboxamide |
| 46 | 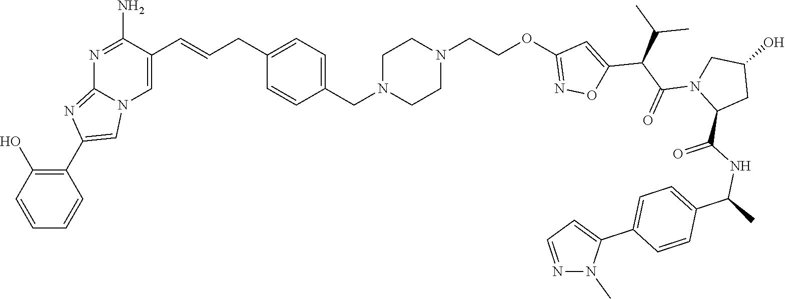 [SMILES: CC(C)[C@@H](C(=O)N1C[C@H](O)C[C@H]1C(=O)N[C@@H](C)c1ccc(-c2ccnn2C)cc1)c1cc(OCCN2CCN(Cc3ccc(C/C=C/c4cn5cc(-c6ccccc6O)nc5nc4N)cc3)CC2)no1] | (2S,4R)-1-[(2R)-2-(3-{2-[4-({4-[(2E)-3- [7-amino-2-(2- hydroxyphenyl)imidazo[1,2-a]pyrimidin- 6-yl]prop-2-en-1- yl]phenyl}methyl)piperazin-1- yl]ethoxy}-1,2-oxazol-5-yl)-3- methylbutanoyl]-4-hydroxy-N-[(1S)-1- [4-(1-methyl-1H-pyrazol-5- yl)phenyl]ethyl]pyrrolidine-2- carboxamide |

TABLE 2
Target protein degradation via bifunctional degradation compounds of Table 1

| | DC₅₀ | Dₘₐₓ | | |
|---|---|---|---|---|
| Ex. | (nM)* | (%)** | MH+ | NMR transcript |
| 1 | B | A | 1039.3 | |
| 2 | B | A | 1057.3 | ¹H NMR: (400 MHz, DMSO-d₆) δ: 13.33-13.16 (m, 1H), 9.01- |
| | | | | 8.97 (m, 1H), 8.40 (d, J = 7.6 Hz, 1H), 8.11-8.01 (m, 2H), |
| | | | | 7.83 (dd, J = 1.1, 7.6 Hz, 1H), 7.49-7.42 (m, 2H), 7.41-7.35 |
| | | | | (m, 2H), 7.30-7.24 (m, 1H), 6.98 (d, J = 5.3 Hz, 1H), 6.96- |
| | | | | 6.90 (m, 2H), 6.87 (s, 2H), 6.83 (s, 1H), 6.15-6.07 (m, 1H), |
| | | | | 6.02-5.96 (m, 1H), 5.26-5.18 (m, 1H), 5.11 (br d, J = 2.6 Hz, |
| | | | | 1H), 4.92 (br t, J = 7.3 Hz, 1H), 4.37 (t, J = 7.7 Hz, 1H), 4.33- |
| | | | | 4.25 (m, 2H), 3.72 (dd, J = 4.3, 10.1 Hz, 1H), 3.66-3.53 (m, |
| | | | | 3H), 3.46-3.40 (m, 1H), 3.30-3.20 (m, 4H), 2.79-2.70 (m, |
| | | | | 2H), 2.46 (s, 3H), 2.33-2.16 (m, 5H), 2.11 (br d, J = 7.3 Hz, |
| | | | | 2H), 2.06-1.94 (m, 3H), 1.86-1.66 (m, 6H), 1.65-1.54 (m, |
| | | | | 1H), 1.48-1.37 (m, 5H), 1.18-1.06 (m, 2H), 0.98-0.93 (m, |
| | | | | 3H), 0.87-0.76 (m, 3H) |
| 3 | A | A | 1108.5 | ¹H NMR: (400 MHz, MeOD-d₄) δ: 8.88 (s, 1H), 8.44 (br s, |
| | | | | 2H), 7.94-7.73 (m, 2H), 7.61-7.50 (m, 1H), 7.49-7.24 (m, |
| | | | | 6H), 7.07-6.85 (m, 2H), 6.23-6.00 (m, 1H), 5.08-4.91 (m, |
| | | | | 2H), 4.77-4.36 (m, 5H), 3.92-3.60 (m, 10H), 3.55-3.40 (m, |
| | | | | 2H), 3.21-2.99 (m, 4H), 2.96-2.82 (m, 4H), 2.75-2.60 (m, |
| | | | | 4H), 2.49 (s, 3H), 2.42-2.14 (m, 6H), 2.10-1.78 (m, 8H), 1.65- |
| | | | | 1.49 (m, 3H), 1.45-1.24 (m, 2H), 1.06 (d, J = 6.4 Hz, 3H), |
| | | | | 0.95-0.82 (m, 3H) |
| 4 | A | A | 1137.7 | ¹H NMR: (400 MHz, DMSO-d₆) δ: 8.98 (s, 1H), 8.39 (d, J = 7.6 |
| | | | | Hz, 1H), 8.14 (s, 1H), 7.94 (d, J = 7.6 Hz, 1H), 7.83 (d, J = 6.0 |
| | | | | Hz, 1H), 7.63 (s, 1H), 7.45-7.43 (m, 2H), 7.38-7.35 (m, 2H), |
| | | | | 7.26-7.22 (m, 1H), 6.90-6.87 (m, 2H), 6.52-6.34 (m, 3H), |
| | | | | 6.11-5.99 (m, 1H), 5.73 (s, 1H), 5.19-5.17 (m, 1H), 5.10- |
| | | | | 5.09 (m, 1H), 4.93-4.89 (m, 1H), 4.64 (m, 4H), 4.38-4.28 (m, |
| | | | | 2H), 3.77-3.54 (m, 9H), 2.90-2.61 (m, 7H), 2.45 (s, 3H), 2.63- |
| | | | | 2.20 (m, 9H), 1.82-1.64 (m, 7H), 1.52-1.37 (m, 5H), 1.16- |
| | | | | 1.07 (m, 2H), 0.97-0.92 (m, 3H), 0.83-0.78 (m, 3H) |
| 5 | A | A | 1095.7 | ¹H NMR: (400 MHz, MeOD-d₄) δ: 8.88 (s, 1H), 8.51 (s, 1H), |
| | | | | 8.13-8.03 (m, 1H), 7.73 (d, J = 7.2 Hz, 1H), 7.50-7.36 (m, |
| | | | | 4H), 7.30-7.22 (m, 1H), 6.99-6.88 (m, 2H), 6.16-6.02 (m, |
| | | | | 1H), 5.08-4.99 (m, 1H), 4.58-4.37 (m, 3H), 4.26-4.22 (m, |
| | | | | 1H), 3.89-3.52 (m, 7H), 3.36-3.32 (m, 2H), 3.16-3.12 (m, 2H), |
| | | | | 2.99-2.67 (m, 8H), 2.58-2.30 (m, 12H), 2.27-2.14 (m, 3H), |
| | | | | 2.04-1.90 (m, 6H), 1.85-1.67 (m, 6H), 1.60-1.50 (m, 3H), |
| | | | | 1.38-1.26 (m, 2H), 1.05 (d, J = 6.4 Hz, 3H), 0.94-0.86 (m, |
| | | | | 3H) |
| 6 | A | A | 1121.7 | |
| 7 | A | A | 1121.7 | |
| 8 | A | A | 1108.7 | ¹H NMR: (400 MHz, MeOD-d₄) δ: 8.89 (s, 1H), 8.58-8.34 (m, |
| | | | | 3H), 7.85-7.74 (m, 1H), 7.52-7.23 (m, 6H), 7.32-7.22 (m, |
| | | | | 1H), 6.99-6.90 (m, 3H), 6.15-6.05 (m, 1H), 5.55-5.41 (m, |
| | | | | 1H), 5.12-5.02 (m, 1H), 4.60-4.41 (m, 3H), 3.87 (dd, J = 4.0, |
| | | | | 10.8 Hz, 1H), 3.76-3.60 (m, 7H), 3.52-3.35 (m, 3H), 3.18- |
| | | | | 3.10 (m, 2H), 3.02-2.59 (m, 13H), 2.51-2.49 (m, 3H), 2.45- |
| | | | | 2.33 (m, 1H), 2.25-2.16 (m, 3H), 2.09-1.96 (m, 5H), 1.93- |
| | | | | 1.74 (m, 5H), 1.63-1.52 (m, 3H), 1.37-1.25 (m, 2H), 1.07 (d, |
| | | | | J = 6.4 Hz, 3H), 0.96-0.89 (m, 3H) |
| 9 | A | A | 1097.7 | ¹H NMR: (400 MHz, DMSO-d₆) δ: 14.55 (br s, 1H), 9.03- |
| | | | | 8.73 (m, 1H), 8.40 (d, J = 7.6 Hz, 1H), 7.95-7.83 (m, 1H), 7.44 |
| | | | | (d, J = 8.0 Hz, 2H), 7.40-7.33 (m, 2H), 7.25-7.19 (m, 1H), |
| | | | | 7.11 (s, 1H), 6.92-6.82 (m, 2H), 6.10 (s, 1H), 6.00 (s, 2H), |
| | | | | 5.14-4.99 (m, 1H), 4.91 (br t, J = 7.2 Hz, 1H), 4.36 (t, J = 7.6 |
| | | | | Hz, 1H), 4.28 (br s, 1H), 4.13-4.03 (m, 1H), 3.83 (d, J = 10.0 |
| | | | | Hz, 2H), 3.71 (br dd, J = 4.0, 10.4 Hz, 1H), 3.65-3.52 (m, 3H), |
| | | | | 3.47-3.38 (m, 1H), 3.30 (s, 2H), 3.26-3.20 (m, 1H), 2.78- |
| | | | | 2.58 (m, 6H), 2.53 (br s, 1H), 2.46 (s, 3H), 2.44-2.36 (m, 2H), |
| | | | | 2.25 (br d, J = 6.4 Hz, 5H), 2.15-2.04 (m, 5H), 2.03-1.87 (m, |
| | | | | 5H), 1.87-1.55 (m, 7H), 1.50 (br s, 2H), 1.46-1.31 (m, 5H), |
| | | | | 1.17-1.01 (m, 3H), 0.95 (br d, J = 6.4 Hz, 3H), 0.79 (d, J = 6.8 |
| | | | | Hz, 3H) |
| 10 | A | A | 1108.7 | ¹H NMR: (400 MHz, DMSO-d₆) δ = 8.98 (s, 1H), |
| | | | | 8.40 (d, J = 8.0 Hz, 1H), 8.13 (s, 2H), 7.92 (d, J = 6.8 Hz, 1H), |
| | | | | 7.74 (s, 1H), 7.50 (s, 1H), 7.44 (d, J = 8.4 Hz, 2H), 7.36 (d, J = |
| | | | | 8.0 Hz, 2H), 7.30 (s, 1H), 7.24 (t, J = 7.6 Hz, 1H), 6.89 (d, J = |
| | | | | 7.6 Hz, 2H), 6.11 (s, 1H), 5.96 (s, 2H), 5.12 (s, 1H), 4.91 (s, |
| | | | | 1H), 4.82 (s, 1H), 4.36 (d, J = 7.6 Hz, 2H), 4.28 (s, 2H), 3.70 (s, |
| | | | | 2H), 3.65-3.62 (m, 3H), 3.58 (s, 2H), 3.55-3.53 (m, 4H), 3.01 |
| | | | | (s, 2H), 2.98 (s, 2H), 2.82 (s, 2H), 2.75 (d, J = 10.4 Hz, 2H), |
| | | | | 2.67 (s, 2H), 2.32 (s, 3H), 2.23 (s, 3H), 2.05 (s, 3H), 2.03-1.98 |
| | | | | (m, 2H), 1.84 (s, 3H), 1.72 (d, J = 9.6 Hz, 3H), 1.52 (s, 2H), |
| | | | | 1.45 (d, J = 7.6 Hz, 1H), 1.37 (d, J = 6.4 Hz, 3H), 1.12 (d, J = |
| | | | | 9.6 Hz, 2H), 0.94 (d, J = 5.6 Hz, 3H), 0.78 (d, J = 5.6 Hz, 3H) |
| 11 | B | A | 1141.7 | |
| 12 | B | A | 1108.7 | ¹H NMR: (400 MHz, CD₃OD) δ = 8.88 (s, 1H), 7.73 (d, J = 2.8 |
| | | | | Hz, 1H), 7.60 (s, 1H), 7.47-7.44 (m, 1H), 7.42 (d, J = 2.8 Hz, |
| | | | | 3H), 7.39-7.36 (m, 1H), 7.26 (t, J = 4.2 Hz, 1H), 7.24-6.93 (m, |
| | | | | 1H), 6.91 (d, J = 2.8 Hz, 2H), 6.11 (s, 1H), 5.04 (d, J = 2.4 Hz, |
| | | | | 1H), 4.53-4.51 (m, 2H), 4.49-4.45 (m, 1H), 4.44 (m, 2H), 4.33 |
| | | | | (s, 2H), 3.86-3.72 (m, 1H), 3.65-3.64 (m, 3H), 3.63-3.60 (m, |
| | | | | 3H), 3.51-3.48 (m, 3H), 3.13(s, 2H), 2.94-2.91 (m, 5H), 2.76- |
| | | | | 2.66 (m, 2H), 2.65-2.64 (m, 2H), 2.53-2.48 (m, 4H), 2.47-2.45 |
| | | | | (m, 3H), 2.10-2.08 (m, 1H), 2.03-2.00 (m, 8H), 1.97-1.85 (m, |
| | | | | 3H), 1.54 (d, J = 2.8 Hz, 3H), 1.37-1.31 (m, 2H), 1.05 (d, J = |
| | | | | 2.8 Hz, 3H), 0.90 (d, J = 2.8 Hz, 3H) |
| 13 | A | A | 1141.8 | |
| 14 | D | NA | 1108.7 | |
| 15 | A | A | 1043.6 | |
| 16 | D | NA | 1044.6 | |
| 17 | B | A | 1044.6 | |
| 18 | C | A | 1111.7 | |
| 19 | B | A | 971.5 | ¹H NMR (400 MHz, DMSO-d₆): δ 8.51-8.48 (m, 1H), 7.94 (d, |
| | | | | J = 7.6 Hz, 1H), 7.85-7.76 (m, 3H), 7.62 (s, 1H), 7.47-7.44 |
| | | | | (m, 2H), 7.25-7.23 (m, 1H), 6.90-6.86 (m, 1H), 6.41 (s, 1H), |
| | | | | 6.35-6.33 (m, 1H), 6.15-6.08 (m, 1H), 5.81-5.55 (m, 1H), |
| | | | | 5.11-4.99 (m, 1H), 4.93-4.87 (m, 1H), 4.64-4.58 (m, 4H), |
| | | | | 4.36-4.33 (m, 1H), 4.32-4.21 (m, 5H), 3.68-3.61 (m, 4H), |
| | | | | 2.67-2.63 (m, 4H), 2.25-2.18 (m, 2H), 2.15-1.95 (m, 1H), |
| | | | | 1.75-1.70 (m, 1H), 1.46-1.33 (m, 3H), 0.96-0.92 (m, 3H), |
| | | | | 0.82-0.77 (m, 3H) |
| 20 | B | A | 1026.6 | ¹H NMR (400 MHz, DMSO-d₆): δ 8.44 (d, J = 7.6 Hz, 1H), |
| | | | | 8.16 (s, 0.5H), 7.96-7.93 (m, 1H), 7.84 (d, J = 6.0 Hz, 1H), |
| | | | | 7.63 (s, 1H), 7.42-7.36 (m, 4H), 7.28-7.23 (m, 2H), 6.90- |
| | | | | 6.87 (m, 3H), 6.43 (s, 2H), 6.35-6.32 (m, 1H), 6.09 (s, 1H), |
| | | | | 5.76 (s, 1H), 5.11-4.98 (m, 1H), 4.97-4.92 (m, 1H), 4.65- |
| | | | | 4.63 (m, 4H), 4.37-4.35 (m, 1H), 4.34-4.27 (m, 3H), 4.26- |
| | | | | 4.21 (m, 2H), 3.69-3.65 (m, 5H), 3.65-3.61 (m, 4H), 2.67- |
| | | | | 2.60 (m, 5H), 2.27 (s, 3H), 2.25-2.21 (m, 1H), 2.12-1.95 (m, |
| | | | | 1H), 1.80-1.76 (m, 1H), 1.47-1.37 (m, 3H), 0.96-0.94 (m, |
| | | | | 3H), 0.83-0.74 (m, 3H) |
| 21 | D | NA | 1026.6 | |
| 22 | A | A | 1141.7 | |
| 23 | | | 1067.7 | |
| 24 | A | A | 1012.6 | ¹H NMR (400 MHz, CD₃OD): δ 7.86-7.79 (m, 2H), 7.73-7.66 |
| | | | | (m, 2H), 7.62 (s, 1H), 7.51-7.43 (m, 2H), 7.32-7.19 (m, 1H), |
| | | | | 6.96-6.87 (m, 2H), 6.39-6.35 (m, 1H), 6.00 (s, 1H), 5.81 (d, J = |
| | | | | 2.0 Hz, 1H), 5.16-5.10 (m, 2H), 4.82-4.79 (m, 1H), 4.78- |
| | | | | 4.75 (m, 2H), 4.51-4.49 (m, 1H), 4.52-4.46 (m, 1H), 4.45- |
| | | | | 4.30 (m, 3H), 3.86-3.81 (m, 1H), 3.71 (s, 4H), 3.68-3.64 (m, |
| | | | | 1H), 3.66-3.58 (m, 1H), 3.45-3.37 (m, 1H), 3.01-2.91 (m, |
| | | | | 2H), 2.88-2.81 (m, 2H), 2.81-2.74 (m, 1H), 2.50-2.24 (m, |
| | | | | 7H), 2.23-2.11 (m, 1H), 1.97-1.79 (m, 3H), 1.72-1.56 (m, |
| | | | | 2H), 1.51-1.48 (m, 2H), 1.38-1.26 (m, 1H), 1.05-1.03 (m, |
| | | | | 3H), 0.94-0.83 (m, 3H) |
| 25 | | | 1067.7 | |
| 26 | A | A | 1141.8 | |
| 27 | B | A | 1154.9 | |
| 28 | A | A | 981.3 | ¹H NMR: (400 MHz, MeOD-d₄) δ 8.78 (s, 1H), 8.58-8.27 (m, |
| | | | | 2H), 7.85-7.44 (m, 2H), 7.42-7.24 (m, 4H), 7.22-7.02 (m, |
| | | | | 1H), 6.90-6.73 (m, 2H), 6.11-5.94 (m, 1H), 4.95 (q, J = 7.2 |
| | | | | Hz, 1H), 4.71-4.64 (m, 1H), 4.47-4.27 (m, 3H), 3.76 (dd, J = |
| | | | | 4.0, 10.8 Hz, 1H), 3.70-3.43 (m, 5H), 3.23 (br s, 2H), 3.16- |
| | | | | 3.00 (m, 2H), 2.95-2.74 (m, 4H), 2.50-2.18 (m, 8H), 2.17- |
| | | | | 2.05 (m, 1H), 2.03-1.68 (m, 8H), 1.53-1.38 (m, 3H), 1.34- |
| | | | | 1.20 (m, 2H), 1.01-0.93 (m, 3H), 0.88-0.76 (m, 3H) |
| 29 | A | A | 1011.3 | ¹H NMR (400 MHz, DMSO-d₆) δ: 12.07 (br s, 1H), 9.05-8.93 |
| | | | | (m, 1H), 8.81 (s, 1H), 8.40 (d, J = 7.6 Hz, 1H), 7.87 (s, 1H), |
| | | | | 7.74 (dd, J = 1.2, 7.6 Hz, 1H), 7.50-7.40 (m, 2H), 7.39-7.32 |
| | | | | (m, 2H), 7.25-7.01 (m, 2H), 6.90-6.79 (m, 2H), 6.10 (s, 1H), |
| | | | | 5.11 (br s, 1H), 4.91 (t, J = 7.2 Hz, 1H), 4.42-4.32 (m, 3H), |
| | | | | 4.32-4.18 (m, 3H), 3.71 (br dd, J = 4.4, 10.4 Hz, 1H), 3.64- |
| | | | | 3.52 (m, 3H), 3.47-3.39 (m, 1H), 3.27-3.19 (m, 1H), 2.79- |
| | | | | 2.53 (m, 5H), 2.45 (s, 3H), 2.30-2.05 (m, 7H), 2.05-1.88 (m, |
| | | | | 3H), 1.83-1.58 (m, 6H), 1.47-1.32 (m, 5H), 1.24-1.01 (m, |
| | | | | 2H), 1.00-0.90 (m, 3H), 0.85-0.70 (m, 3H) |
| 30 | D | NA | 789.2 | ¹H NMR (400 MHz, DMSO-d₆) δ: 9.03-8.91 (m, 1H), 8.87- |
| | | | | 8.77 (m, 1H), 8.43-8.34 (m, 1H), 8.04-7.89 (m, 1H), 7.76 (br |
| | | | | d, J = 6.8 Hz, 1H), 7.51-7.39 (m, 3H), 7.39-7.30 (m, 2H), |
| | | | | 7.27-7.17 (m, 1H), 7.01-6.85 (m, 2H), 6.22-6.06 (m, 1H), |
| | | | | 5.12 (d, J = 3.6 Hz, 1H), 5.06-4.85 (m, 1H), 4.59-4.41 (m, |
| | | | | 1H), 4.33-4.24 (m, 3H), 3.77 (d, J = 8.8 Hz, 1H), 3.61-3.53 |
| | | | | (m, 1H), 3.51-3.41 (m, 2H), 2.67-2.61 (m, 2H), 2.46-2.43 |
| | | | | (m, 3H), 2.08-2.02 (m, 2H), 1.83-1.74 (m, 1H), 1.45-1.33 |
| | | | | (m, 3H), 0.97 (d, J = 6.8 Hz, 3H), 0.89-0.81 (m, 3H) |
| 31 | D | NA | 789.2 | ¹H NMR (400 MHz, DMSO-d₆) δ: 12.17 (br s, 1H), 9.02-8.97 |
| | | | | (m, 1H), 8.77-8.73 (m, 1H), 8.45 (d, J = 7.6 Hz, 1H), 7.91- |
| | | | | 7.84 (m, 1H), 7.79-7.68 (m, 1H), 7.46-7.42 (m, 2H), 7.40- |
| | | | | 7.35 (m, 2H), 7.14 (ddd, J = 1.6, 7.2, 8.4 Hz, 1H), 6.89-6.81 |
| | | | | (m, 2H), 6.13 (s, 1H), 5.17 (d, J = 3.6 Hz, 1H), 4.98-4.86 (m, |
| | | | | 1H), 4.38 (t, J = 8.0 Hz, 1H), 4.30 (br t, J = 6.0 Hz, 3H), 3.76- |
| | | | | 3.62 (m, 2H), 2.67-2.62 (m, 2H), 2.47-2.44 (m, 3H), 2.31- |
| | | | | 2.17 (m, 1H), 2.11-1.99 (m, 3H), 1.78 (ddd, J = 4.8, 8.0, 12.8 |
| | | | | Hz, 1H), 1.44-1.35 (m, 3H), 1.00-0.94 (m, 3H), 0.85-0.79 |
| | | | | (m, 3H) |
| 32 | A | A | 994.4 | ¹H NMR (400 MHz, DMSO-d₆) δ: 9.72 (s, 1H), 8.89 (s, 1H), |
| | | | | 8.41 (d, J = 7.6 Hz, 1H), 7.99 (s, 1H), 7.79 (d, J = 7.6 Hz, 1H), |
| | | | | 7.52-7.43 (m, 3H), 7.42-7.36 (m, 2H), 7.22 (s, 1H), 7.04- |
| | | | | 6.86 (m, 2H), 6.39-6.35 (m, 1H), 6.19-6.03 (m, 1H), 5.13 (d, |
| | | | | J = 2.8 Hz, 1H), 4.93 (m, 1H), 4.45-4.20 (m, 6H), 3.84 (s, 3H), |
| | | | | 3.77-3.34 (m, 8H), 3.07-2.85 (m, 4H), 2.84-2.73 (m, 2H), |
| | | | | 2.32-2.11 (m, 5H), 2.01 (s, 4H), 1.95-1.59 (m, 6H), 1.53- |
| | | | | 1.11 (m, 6H), 1.00-0.92 (m, 3H), 0.85-0.76 (m, 3H). |
| 33 | A | A | 964.4 | ¹H NMR (400 MHz, DMSO-d₆) δ: 8.77-8.71 (m, 1H), 8.41 |
| | | | | (d, J = 7.6 Hz, 1H), 7.86 (s, 1H), 7.73 (d, J = 6.8 Hz, 1H), 7.52- |
| | | | | 7.45 (m, 3H), 7.39 (d, J = 8.0 Hz, 2H), 7.18-7.11 (m, 1H), |
| | | | | 6.90-6.82 (m, 2H), 6.40-6.36 (m, 1H), 6.11 (s, 1H), 5.17- |
| | | | | 4.86 (m, 2H), 4.45-4.34 (m, 2H), 4.29 (s, 1H), 3.85 (s, 3H), |
| | | | | 3.72 (dd, J = 10.4, 4.4 Hz, 1H), 3.65-3.54 (m, 3H), 3.48-3.40 |
| | | | | (m, 1H), 3.32-3.25 (m, 2H), 2.77-2.65 (m, 4H), 2.49-2.41 |
| | | | | (m, 3H), 2.36-1.84 (m, 10H), 1.83-1.60 (m, 6H), 1.49- |
| | | | | 1.36 (m, 5H), 1.10 (d, J = 11.6 Hz, 2H), 1.01-0.92 (m, 3H), |
| | | | | 0.86-0.77 (m, 3H) |
| 34 | D | NA | 914.5 | ¹H NMR (400 MHz, DMSO-d₆) δ: 9.01-8.97 (m, 1H), 8.93- |
| | | | | 8.85 (m, 1H), 8.23 (d, J = 8.0 Hz, 1H), 8.13 (s, 1H), 7.95 (s, 1H), |
| | | | | 7.76 (d, J = 6.4 Hz, 1H), 7.50-7.28 (m, 5H), 7.20 (s, 1H), 6.91 |
| | | | | (d, J = 8.8 Hz, 2H), 6.16-6.12 (m, 1H), 5.10 (d, J = 3.6 Hz, 1H), |
| | | | | 4.92-4.81 (m, 1H), 4.36 (s, 2H), 4.32-4.23 (m, 3H), 3.68 (d, |
| | | | | J = 8.8 Hz, 1H), 3.52 (d, J = 5.2 Hz, 1H), 3.42 (d, J = 4.0 Hz, 6H), |
| | | | | 2.96-2.82 (m, 3H), 2.46 (s, 1H), 2.44 (s, 2H), 2.24 (d, J = 4.0 |
| | | | | Hz, 3H), 2.16 (dd, J = 6.4, 11.6 Hz, 3H), 2.08-2.00 (m, 1H), |
| | | | | 1.85-1.75 (m, 3H), 1.47-1.42 (m, 2H), 1.35 (d, J = 7.2 Hz, |
| | | | | 3H), 0.96 (s, 1H), 0.94 (s, 1H), 0.83-0.73 (m, 4H) |
| 35 | B | A | 914.5 | ¹H NMR (400 MHz, DMSO-d₆) δ: 8.98 (s, 1H), 8.82 (s, 1H), |
| | | | | 8.40 (d, J = 7.6 Hz, 1H), 8.28 (s, 1H), 7.88 (s, 1H), 7.75 (d, J = 8.0 |
| | | | | Hz, 1H), 7.50-7.31 (m, 5H), 7.20-7.08 (m, 2H), 6.89-6.81 |
| | | | | (m, 2H), 6.13 (s, 1H), 4.91 (m, 1H), 4.39-4.34 (m, 3H), 4.32- |
| | | | | 4.26 (m, 3H), 3.71 (dd, J = 4.4, 10.4 Hz, 1H), 3.56 (d, J = 10.0 Hz, |
| | | | | 2H), 3.52-3.49 (m, 2H), 2.92 (m, 2H), 2.46 (s, 4H), 2.30-2.23 |
| | | | | (m, 3H), 2.21-2.13 (m, 3H), 2.02 (m, J = 8.8 Hz, 1H), 1.86- |
| | | | | 1.77 (m, 3H), 1.47-1.41 (m, 2H), 1.38 (d, J = 7.2 Hz, 3H), 0.98- |
| | | | | 0.77 (m, 7H) |
| 36 | A | A | 1078.7 | ¹H NMR (400 MHz, DMSO-d₆) δ: 12.37-11.92 (m, 1H), 9.03- |
| | | | | 8.95 (m, 1H), 8.74 (s, 1H), 8.40 (d, J = 7.6 Hz, 1H), 7.85 (s, |
| | | | | 1H), 7.73 (dd, J = 1.6, 7.6 Hz, 1H), 7.48-7.42 (m, 2H), 7.41- |
| | | | | 7.35 (m, 2H), 7.17-7.11 (m, 1H), 6.92-6.79 (m, 2H), 6.11 (s, |
| | | | | 1H), 4.92 (quin, J = 7.2 Hz, 1H), 4.37 (t, J = 7.6 Hz, 1H), 4.29 |
| | | | | (br s, 1H), 3.80-3.68 (m, 2H), 3.65-3.54 (m, 3H), 3.48-3.39 |
| | | | | (m, 1H), 3.29-3.22 (m, 1H), 2.81-2.62 (m, 6H), 2.48-2.44 |
| | | | | (m, 5H), 2.40-2.34 (m, 2H), 2.27-2.17 (m, 2H), 2.13-1.93 |
| | | | | (m, 5H), 1.85-1.50 (m, 14H), 1.47-1.35 (m, 5H), 1.33-1.21 |
| | | | | (m, 2H), 1.17-1.05 (m, 2H), 1.00-0.92 (m, 3H), 0.88-0.76 |
| | | | | (m, 3H) |
| 37 | A | A | 1078.7 | ¹H NMR (400 MHz, DMSO-d₆) δ: 9.04-8.95 (m, 1H), 8.74 (s, |
| | | | | 1H), 8.40 (d, J = 7.6 Hz, 1H), 7.85 (s, 1H), 7.73 (br d, J = 8.0 |
| | | | | Hz, 1H), 7.44 (d, J = 8.4 Hz, 2H), 7.39-7.35 (m, 2H), 7.14 (br |
| | | | | t, J = 7.6 Hz, 1H), 6.89-6.81 (m, 2H), 6.11 (s, 1H), 4.92 (quin, |
| | | | | J = 7.2 Hz, 1H), 4.37 (t, J = 8.0 Hz, 1H), 4.29 (br s, 1H), 4.14- |
| | | | | 4.03 (m, 1H), 3.77-3.69 (m, 1H), 3.64-3.54 (m, 3H), 3.42 (br |
| | | | | d, J = 10.0 Hz, 1H), 3.26-3.19 (m, 1H), 2.87 (br d, J = 10.8 |
| | | | | Hz, 2H), 2.74 (br d, J = 12.0 Hz, 2H), 2.67-2.62 (m, 2H), 2.46 |
| | | | | (s, 5H), 2.14-1.91 (m, 10H), 1.85-1.51 (m, 14H), 1.46-1.36 |
| | | | | (m, 5H), 1.28 (br d, J = 9.2 Hz, 2H), 1.16-1.06 (m, 2H), 0.98- |
| | | | | 0.93 (m, 3H), 0.83-0.77 (m, 3H) |
| 38 | B | A | 997.4 | ¹H NMR (400 MHz, DMSO-d₆) δ: 8.98 (s, 1H), 8.89-8.67 (m, |
| | | | | 1H), 8.39 (d, J = 7.6 Hz, 1H), 8.16 (s, 1H), 8.03-7.58 (m, 4H), |
| | | | | 7.49-7.31 (m, 4H), 7.29-7.06 (m, 1H), 6.92-6.79 (m, 2H), |
| | | | | 6.16-5.93 (m, 1H), 4.95-4.88 (m, 1H), 4.36 (t, J = 7.6 Hz, |
| | | | | 1H), 4.31-4.07 (m, 3H), 3.82-3.49 (m, 6H), 3.44-3.26 (m, |
| | | | | 2H), 2.93-2.59 (m, 4H), 2.53-2.50 (m, 2H), 2.45 (s, 3H), 2.35- |
| | | | | 2.09 (m, 5H), 2.06-1.95 (m, 1H), 1.87-1.61 (m, 6H), 1.53- |
| | | | | 1.42 (m, 2H), 1.37 (d, J = 7.2 Hz, 2H), 1.18-1.02 (m, 2H), 0.99- |
| | | | | 0.87 (m, 3H), 0.84-0.72 (m, 3H) |
| 39 | A | A | 982.5 | ¹H NMR (400 MHz, MeOD-d₄) δ: 8.59-8.52 (m, 1H), 7.80 (s, |
| | | | | 1H), 7.74-7.69 (m, 1H), 7.53-7.45 (m, 5H), 7.19-7.12 (m, |
| | | | | 1H), 6.92-6.84 (m, 2H), 6.13-6.05 (m, 1H), 5.07 (dd, J = 8.0, |
| | | | | 12.0 Hz, 1H), 4.64-4.46 (m, 5H), 3.91-3.79 (m, 4H), 3.73- |
| | | | | 3.59 (m, 4H), 3.50-3.39 (m, 1H), 2.93-2.76 (m, 4H), 2.55- |
| | | | | 2.37 (m, 4H), 2.28-2.13 (m, 5H), 2.02-1.79 (m, 5H), 1.65- |
| | | | | 1.52 (m, 5H), 1.33-1.17 (m, 3H), 1.07 (d, J = 6.4 Hz, 3H), 0.95- |
| | | | | 0.87 (m, 3H) |
| 40 | B | A | 978.4 | ¹H NMR (400 MHz, DMSO-d₆) δ: 8.87-8.69 (m, 1H), 8.40 (d, |
| | | | | J = 8.0 Hz, 1H), 8.14 (s, 1H), 7.87 (s, 1H), 7.79-7.69 (m, 1H), |
| | | | | 7.45-7.29 (m, 5H), 7.19-7.12 (m, 1H), 6.92-6.80 (m, 2H), |
| | | | | 6.21-6.01 (m, 1H), 5.13 (br d, J = 3.2 Hz, 1H), 5.05-4.91 (m, |
| | | | | 1H), 4.48-4.25 (m, 3H), 3.79-3.46 (m, 10H), 3.18-2.94 (m, |
| | | | | 3H), 2.90-2.57 (m, 6H), 2.48-2.40 (m, 2H), 2.37-2.10 (m, |
| | | | | 4H), 2.09-1.53 (m, 13H), 1.52-1.37 (m, 3H), 1.27-1.12 (m, |
| | | | | 2H), 1.03-0.90 (m, 3H), 0.88-0.74 (m, 3H) |
| 41 | A | A | 1006.4 | ¹H NMR (400 MHz, DMSO-d₆) δ: 12.96-12.79 (m, 1H), 9.04- |
| | | | | 8.96 (m, 1H), 8.41 (d, J = 7.6 Hz, 1H), 8.28 (s, 1H), 8.21 (d, J = |
| | | | | 5.6 Hz, 1H), 7.97-7.92 (m, 1H), 7.49-7.42 (m, 2H), 7.40- |
| | | | | 7.34 (m, 2H), 7.32-7.26 (m, 1H), 7.22 (dd, J = 1.2, 5.2 Hz, |
| | | | | 1H), 7.02 (br d, J = 6.4 Hz, 2H), 6.97-6.92 (m, 2H), 6.85- |
| | | | | 6.72 (m, 1H), 6.11 (s, 1H), 5.33-5.21 (m, 1H), 5.15-4.99 (m, |
| | | | | 1H), 4.98-4.88 (m, 1H), 4.43-4.25 (m, 3H), 3.72 (br dd, J = |
| | | | | 4.4, 10.4 Hz, 1H), 3.66-3.53 (m, 3H), 3.48-3.39 (m, 1H), 2.79- |
| | | | | 2.68 (m, 3H), 2.46 (s, 3H), 2.43-2.31 (m, 5H), 2.28-2.16 (m, |
| | | | | 1H), 2.11 (br d, J = 6.8 Hz, 2H), 2.06-1.93 (m, 3H), 1.83- |
| | | | | 1.61 (m, 6H), 1.49-1.36 (m, 5H), 1.17-1.04 (m, 2H), 1.00- |
| | | | | 0.92 (m, 3H), 0.85-0.77 (m, 3H) |
| 42 | A | A | 1020.4 | ¹H NMR (400 MHz, DMSO-d₆) δ: 12.51 (br s, 1H), 8.54 (s, |
| | | | | 1H), 8.42 (d, J = 7.6 Hz, 1H), 8.10 (s, 1H), 7.90 (s, 1H), 7.75- |
| | | | | 7.68 (m, 2H), 7.53-7.35 (m, 5H), 7.21-7.08 (m, 1H), 6.93- |
| | | | | 6.67 (m, 4H), 6.40-6.36 (m, 1H), 6.11 (s, 1H), 5.11 (br s, 1H), |
| | | | | 4.94 (t, J = 7.2 Hz, 1H), 4.38 (t, J = 7.6 Hz, 1H), 4.33-4.15 |
| | | | | (m, 4H), 3.85 (s, 3H), 3.72 (br dd, J = 4.8, 10.4 Hz, 1H), 3.66- |
| | | | | 3.53 (m, 3H), 3.48-3.40(m, 1H), 3.29-3.20 (m, 1H), 2.79- |
| | | | | 2.60 (m, 5H), 2.25-1.92 (m, 10H), 1.84-1.54 (m, 6H), 1.49- |
| | | | | 1.32 (m, 5H), 1.10 (br d, J = 11.2 Hz, 2H), 1.01-0.92 (m, 3H), |
| | | | | 0.87-0.75 (m, 3H) |
| 43 | B | A | 1037.4 | ¹H NMR (400 MHz, DMSO-d₆) δ: 9.00-8.97 (m, 1H), 8.86 (s, |
| | | | | 1H), 8.39 (d, J = 7.6 Hz, 1H), 8.14 (s, 1H), 8.00-7.87 (m, 3H), |
| | | | | 7.78 (s, 1H), 7.73 (dd, J = 1.6, 8.0 Hz, 1H), 7.46-7.41 (m, 2H), |
| | | | | 7.39-7.33 (m, 2H), 7.18-7.10 (m, 1H), 6.87-6.82 (m, 2H), |
| | | | | 6.12-5.98 (m, 1H), 4.91 (quin, J = 7.2 Hz, 1H), 4.36 (t, J = 8.0 |
| | | | | Hz, 1H), 4.28 (br s, 1H), 4.30-4.26 (m, 1H), 4.12 (br d, J = 8.0 |
| | | | | Hz, 2H), 3.71 (dd, J = 4.4, 10.4 Hz, 2H), 3.61 (d, J = 12.8 Hz, |
| | | | | 3H), 3.56 (d, J = 9.6 Hz, 2H), 2.81-2.68 (m, 5H), 2.45 (s, 3H), |
| | | | | 2.27-2.12 (m, 5H), 2.08-1.97 (m, 5H), 1.83-1.75 (m, 3H), |
| | | | | 1.71 (d, J = 12.0 Hz, 3H), 1.45 (d, J = 7.2 Hz, 3H), 1.38 (d, J = 7.2 |
| | | | | Hz, 3H), 1.11 (d, J = 11.6 Hz, 2H), 0.99-0.91 (m, 3H), 0.85- |
| | | | | 0.76 (m, 3H) |
| 44 | A | A | 1063.5 | ¹H NMR (400 MHz, DMSO-d₆) δ: 12.54 (br s, 1H), 8.62 (s, |
| | | | | 1H), 8.42 (d, J = 7.6 Hz, 1H), 7.85 (s, 1H), 7.68 (d, J = 7.6 Hz, |
| | | | | 1H), 7.51-7.44 (m, 3H), 7.39 (d, J = 8.4 Hz, 2H), 7.13 (t, J = 7.2 |
| | | | | Hz, 1H), 6.99 (br s, 2H), 6.88-6.81 (m, 2H), 6.46-6.41 (m, |
| | | | | 1H), 6.38-6.36(m, 1H), 6.24-6.13 (m, 1H), 6.10 (s, 1H), 5.12 |
| | | | | (br s, 1H), 4.98-4.87 (m, 1H), 4.37 (t, J = 7.6 Hz, 1H), 4.29 (br |
| | | | | s, 1H), 4.08 (br s, 1H), 3.85 (s, 3H), 3.72 (br dd, J = 4.4, 10.4 Hz, |
| | | | | 1H), 3.65-3.51 (m, 3H), 3.48-3.39 (m, 2H), 3.23 (br s, 1H), |
| | | | | 2.85 (br d, J = 9.6 Hz, 2H), 2.77-2.66 (m, 4H), 2.26-1.88 (m, |
| | | | | 13H), 1.84-1.55 (m, 10H), 1.39 (br d, J = 7.2 Hz, 5H), 1.25- |
| | | | | 1.03 (m, 4H), 1.00-0.91 (m, 3H), 0.85-0.75 (m, 3H) |
| 45 | A | A | 996.4 | ¹H NMR (400 MHz, DMSO-d₆) δ: 8.68 (s, 1H), 8.42 (br d, |
| | | | | J = 7.6 Hz, 1H), 8.16 (br s, 1H), 7.91-7.85 (m, 1H), 7.74-7.59 |
| | | | | (m, 1H), 7.52-7.43 (m, 3H), 7.39 (d, J = 8.4 Hz, 2H), 7.25- |
| | | | | 7.11 (m, 1H), 7.07 (s, 1H), 6.93-6.76 (m, 2H), 6.75-6.51 (m, |
| | | | | 1H), 6.40-6.35 (m, 1H), 6.31-6.20 (m, 1H), 6.13 (s, 1H), 4.94 |
| | | | | (quin, J = 7.2 Hz, 1H), 4.37 (br t, J = 8.0 Hz, 1H), 4.32-4.22 (m, |
| | | | | 2H), 4.20-4.15 (m, 1H), 4.02 (br d, J = 5.6 Hz, 2H), 3.85 (s, |
| | | | | 3H), 3.78-3.69 (m, 2H), 3.65-3.55 (m, 5H), 3.47-3.44 (m, |
| | | | | 2H), 2.83 (br s, 2H), 2.80-2.66 (m, 2H), 2.40-1.97 (m, 9H), |
| | | | | 1.86-1.64 (m, 6H), 1.55-1.35 (m, 5H), 1.13 (br d, J = 12.4 Hz, |
| | | | | 2H), 1.02-0.90 (m, 3H), 0.89-0.70 (m, 3H) |
| 46 | B | A | 948.4 | ¹H NMR (400 MHz, DMSO-d₆) δ: 8.69 (s, 1H), 8.40 (d, J = 7.6 |
| | | | | Hz, 1H), 7.92 (s, 1H), 7.72 (br d, J = 7.6 Hz, 1H), 7.54-7.42 |
| | | | | (m, 4H), 7.42-7.27 (m, 6H), 7.24-7.10 (m, 1H), 6.98-6.77 |
| | | | | (m, 2H), 6.67-6.48 (m, 1H), 6.39-6.27 (m, 2H), 6.15-5.89 |
| | | | | (m, 1H), 5.16-4.84 (m, 2H), 4.42-4.21 (m, 4H), 3.84 (s, 3H), |
| | | | | 3.77-3.61 (m, 3H), 3.57 (br d, J = 7.2 Hz, 2H), 3.51-3.36 (m, |
| | | | | 5H), 3.11-3.09 (m, 1H), 3.14-2.54 (m, 8H), 2.29-2.12 (m, |
| | | | | 1H), 2.10-1.95 (m, 1H), 1.94-1.71 (m, 1H), 1.37 (d, J = 7.2 |
| | | | | Hz, 3H), 0.96 (br d, J = 6.4 Hz, 3H), 0.79 (d, J = 6.8 Hz, 3H) |

*DC50 (nM):
A < 2.5
2.5 ≤ B < 10
10 ≤ C < 30
D ≥ 30
NA not calculated/no curve fit
**Dmax (% degraded):
A > 75
50 < B ≤ 75
C ≤ 50
NA not calculated/no curve fit

TABLE 3
Novel ligands of the BRM protein

| | | BRM K_{d} | | |
|---|---|---|---|---|
| Ex. No. | Structure | (nM)^{†} | MH⁺ | NMR transcript |
| BRM1 | 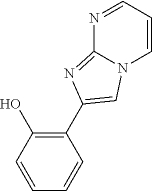 [SMILES: Oc1ccccc1-c1cn2cccnc2n1] | B | 212.1 | ¹H NMR (400 MHz, MeOD) δ 8.89 (d, J = 5.2 Hz, 1H), 8.55 (d, J = 2.4 Hz, 1H), 8.32 (s, 1H), 7.89 (d, J = 7.6 Hz, 1H), 7.22-7.20 (m, 1H), 7.12- 7.07 (m, 1H), 6.95-6.90 (m, 2H) |
| BRM2 | 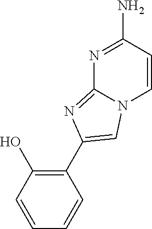 [SMILES: Nc1ccn2cc(-c3ccccc3O)nc2n1] | A | 227.1 | 1H-NMR (400 MHz, DMSO) δ 8.43 (d, J = 7.2 Hz, 1H), 7.90 (s, 1H), 7.69-7.66 (m, 1H), 7.14-7.10 (m, 1H), 7.04 (s, 2H), 6.85-6.81 (m, 2H), 6.35 (d, J = 7.2 Hz, 1H) |
| BRM3 | 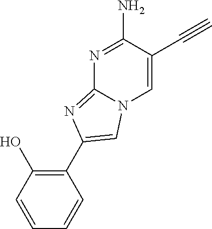 [SMILES: C#Cc1cn2cc(-c3ccccc3O)nc2nc1N] | A | 251.1 | 1H NMR (400 MHz, DMSO- d₆) δ 12.19-11.90 (m, 1H), 8.85 (s, 1H), 7.88 (s, 1H), 7.78-7.72 (m, 1H), 7.18- 7.10 (m, 1H), 7.14 (s, 2H), 6.86 (d, J = 8.4 Hz, 2H), 4.61 (s, 1H) |
| BRM4 | 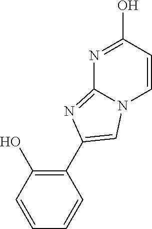 [SMILES: Oc1ccn2cc(-c3ccccc3O)nc2n1] | D | 228.1 | ¹H NMR: (400 MHz, DMSO- d₆) δ: 8.53 (d, J = 7.6 Hz, 1H), 8.00 (s, 1H), 7.84 (dd, J = 1.6, 8.0 Hz, 1H), 7.31-7.10 (m, 1H), 6.95 (dd, J = 0.8, 8.0 Hz, 1H), 6.92-6.86 (m, 1H), 6.28 (br d, J = 7.6 Hz, 1H) |
| BRM5 | 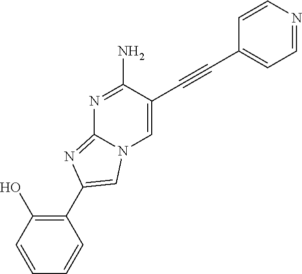 [SMILES: Nc1nc2nc(-c3ccccc3O)cn2cc1C#Cc1ccncc1] | C | 328.1 | 1H NMR (400 MHz, DMSO- d6) δ 12.11-11.83 (m, 1H), 8.99 (s, 1H), 8.66 (d, J = 6.0 Hz, 2H), 7.94 (s, 1H), 7.78 (s, 1H), 7.67 (d, J = 6.0 Hz, 2H), 7.53-7.24 (m, 2H), 7.16 (s, 1H), 6.87 (d, J = 8.0 Hz, 2H) |
| BRM6 | 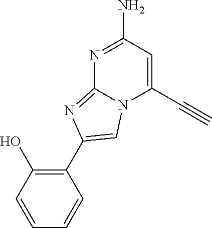 [SMILES: C#Cc1cc(N)nc2nc(-c3ccccc3O)cn12] | C | 251.2 | 1H NMR (400 MHz, CD3OD) δ 8.21 (s, 1H), 7.78 (d, J = 7.6 Hz, 1H), 7.30- 7.15 (m, 1H), 6.98-6.84 (m, 2H), 6.37 (s, 1H), 3.86 (s, 1H) |
| BRM7 | 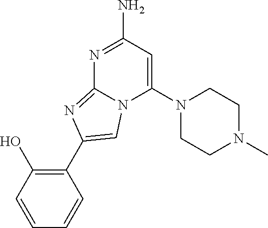 [SMILES: CN1CCN(c2cc(N)nc3nc(-c4ccccc4O)cn23)CC1] | D | 325.2 | ¹H NMR (400 MHz, CD₃OD) δ 8.38 (s, 1H), 8.17 (s, 1H), 7.64 (d, J = 7.6 Hz, 1H), 7.32- 7.23 (m, 1H), 7.05-6.92 (m, 2H), 5.86 (s, 1H), 3.87-3.75 (m, 4H), 2.75-2.63 (m, 4H), 2.46 (s, 3H) |
| BRM8 | 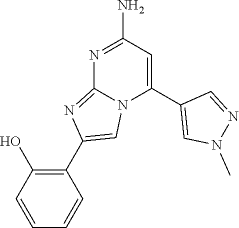 [SMILES: Cn1cc(-c2cc(N)nc3nc(-c4ccccc4O)cn23)cn1] | D | 307.2 | ¹H NMR (400 MHz, CD₃OD) δ 8.24 (s, 2H), 8.07 (s, 1H), 7.73 (dd, J = 7.6, 1.2 Hz, 1H), 7.26 (t, J = 7.6 Hz, 1H), 7.01- 6.92 (m, 2H), 6.58 (s, 1H), 3.97 (s, 3H) |
| BRM9 | 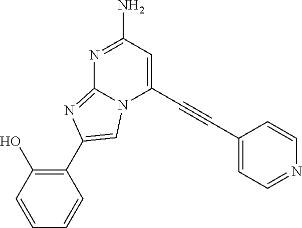 [SMILES: Nc1cc(C#Cc2ccncc2)n2cc(-c3ccccc3O)nc2n1] | D | 328.1 | 1H NMR (400 MHz, CD3OD) δ 8.63 (d, J = 6.0 Hz, 2H), 8.21 (s, 1H), 7.81 (d, J = 7.6 Hz, 1H), 7.66-7.60 (m, 2H), 7.28-7.19 (m, 1H), 6.99-6.88 (m, 2H), 6.45 (s, 1H) |
| BRM10 | 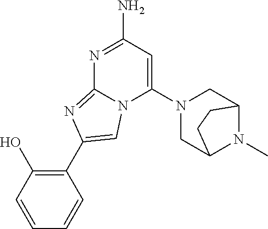 [SMILES: CN1C2CCC1CN(c1cc(N)nc3nc(-c4ccccc4O)cn13)C2] | D | 351.2 | 1H NMR (400 MHz, CD3OD) δ 8.42 (s, 2H), 8.16 (s, 1H), 7.65 (d, J = 7.6 Hz, 1H), 7.31- 7.22 (m, 1H), 7.02-6.92 (m, 2H), 5.81 (s, 1H), 4.35-4.15 (m, 2H), 3.85-3.73 (m, 2H), 3.43-3.35 (m, 2H), 2.69 (s, 3H), 2.30-2.17 (m, 2H), 1.95- 1.83 (m, 2H) |
| BRM11 | 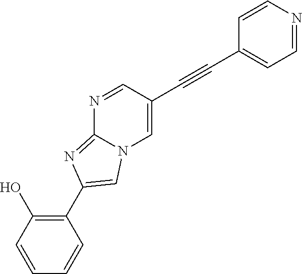 [SMILES: Oc1ccccc1-c1cn2cc(C#Cc3ccncc3)cnc2n1] | D | 313.2 | 1H NMR (400 MHz, DMSO) δ 12.06-11.93 (m, 1H), 8.69 (d, J = 4.8 Hz, 5H), 7.87 (s, 1H), 7.66-7.61 (m, 1H), 7.46- 7.42 (m, 2H), 7.35-7.29 (m, 1H), 7.11-7.05 (m, 1H), 6.97- 6.90 (m, 1H) |
| BRM12 | 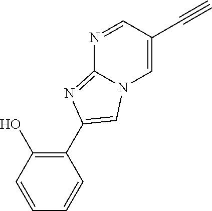 [SMILES: C#Cc1cnc2nc(-c3ccccc3O)cn2c1] | D | 236.2 | ¹H NMR (400 MHz, DMSO) 12.06-11.94 (m, 1H), 8.61 (d, J = 3.2 Hz, 2H), 7.83 (s, 1H), 7.65-7.58 (m, 1H), 7.35- 7.28 (m, 1H), 7.12-7.04 (m, 1H), 6.96-6.88 (m, 1H), 6.92 (s, 1H), 6.95-6.88 (m, 1H), 3.33 (s, 1H) |
| BRM13 | 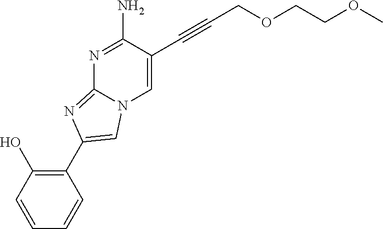 [SMILES: COCCOCC#Cc1cn2cc(-c3ccccc3O)nc2nc1N] | A | 339.1 | ¹H NMR: (400 MHz, DMSO- d₆) δ 8.96 (s, 1H), 8.08 (s, 1H), 7.80 (dd, J = 1.6, 8.0 Hz, 1H), 7.32-7.25 (m, 1H), 7.04 (d, J = 7.6 Hz, 1H), 6.99- 6.94 (m, 1H), 4.48 (s, 2H), 3.71-3.64 (m, 2H), 3.53- 3.50 (m, 2H), 3.28 (s, 3H) |
| BRM14 | 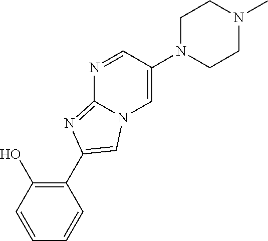 [SMILES: CN1CCN(c2cnc3nc(-c4ccccc4O)cn3c2)CC1] | B | 310.1 | H NMR (400 MHz, Methanol- d₄) δ: 8.54 (s, 1 H), 8.35 (s, 1 H), 8.14 (s, 1 H), 7.79 (d, J = 7.6 Hz, 1 H), 7.19 (t, J = 7.6 Hz, 1 H), 6.87-6.92 (m, 2 H), 3.21-3.25 (m, 4 H), 2.74-2.83 (m, 4 H), 2.48 (s, 3 H) |
| BRM15 | 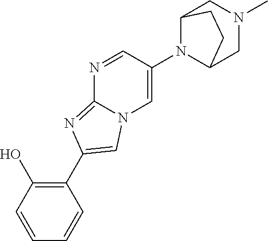 [SMILES: CN1CC2CCC(C1)N2c1cnc2nc(-c3ccccc3O)cn2c1] | C | 336.2 | 1H NMR (400 MHz, Methanol-d₄) δ = 8.49 (d, J = 2.4 Hz, 1H), 8.40-8.31 (m, 2H), 8.10 (s, 1H), 7.82-7.77 (m, 1H), 7.24-7.16 (m, 1H), 6.95-6.87 (m, 2H), 4.35-4.30 (m, 2H), 3.14-3.11 (m, 2H), 3.00-2.97 (m, 2H), 2.57 (s, 3H), 2.23-2.12 (m, 2H), 2.10- 2.02 (m, 2H) |
| BRM16 | 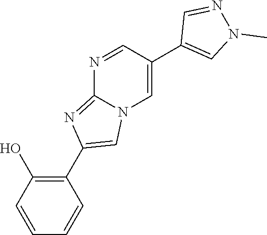 [SMILES: Cn1cc(-c2cnc3nc(-c4ccccc4O)cn3c2)cn1] | B | 292.1 | ¹H NMR (400 MHz, CDCl₃) δ = 8.67 (d, J = 2.4 Hz, 1H), 8.50 (d, J = 2.4 Hz, 1H), 7.83 (s, 1H), 7.78 (s, 1H), 7.72 (s, 1H), 7.60 (dd, J = 7.6, 1.2 Hz, 1H), 7.30-7.25 (m, 1H), 7.07 (dd, J = 8.0, 0.8 Hz, 1H), 6.91 (td, J = 8.0, 1.2 Hz, 1H), 4.02 (s, 3H) |
| BRM17 | 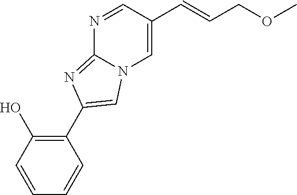 [SMILES: COC/C=C/c1cnc2nc(-c3ccccc3O)cn2c1] | C | 282.1 | ¹H NMR (400 MHz, CDCl₃) δ = 12.15 (br.s, 1H), 8.68 (d, J = 2.4 Hz, 1H), 8.35 (d, J = 2.0 Hz, 1H), 7.78 (s, 1H), 7.58 (dd, J = 7.6, 1.2 Hz, 1H), 7.30-7.28 (m, 1H), 7.06 (dd, J = 8.0, 0.8 Hz, 1H), 6.89 (td, J = 8.0, 0.8 Hz, 1H), 6.59 (d, J = 16.4 Hz, 1H), 6.43 (dt, J = 5.2, 16.4 Hz, 1H), 4.16 (dd, J = 5.2, 1.2 Hz, 2H), 3.46 (s, 3H) |
| BRM18 | 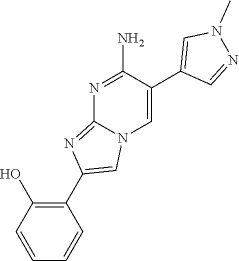 [SMILES: Cn1cc(-c2cn3cc(-c4ccccc4O)nc3nc2N)cn1] | A | 307.2 | ¹H NMR (400 MHz, DMSO- d₆) δ: 13.64 (s, 1H), 10.87 (s, 1H), 8.69 (s, 1H), 8.56-8.39 (m, 1H), 8.12 (s, 1H), 8.06 (s, 1H), 7.85 (d, J = 8.0 Hz, 1H), 7.68 (s, 1H), 7.53-7.32 (m, 1H), 7.31-7.23 (m, 1H), 7.10 (t, J = 7.6 Hz, 1H), 6.96 (t, J = 7.6 Hz, 1H), 3.93 (s, 3H) |
| BRM19 | 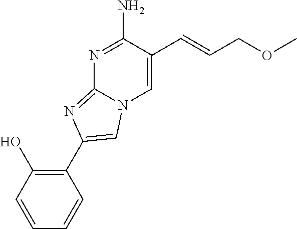 [SMILES: COC/C=C/c1cn2cc(-c3ccccc3O)nc2nc1N] | A | 297.2 | ¹H NMR (400 MHz, Methanol-d₄) δ: 8.50 (s, 1H), 7.80 (s, 1H), 7.69 (d, J = 7.2 Hz, 1H), 7.17-7.10 (m, 1H), 6.88-6.82 (m, 2H), 6.62 (d, J = 16.0 Hz, 1H), 6.32-6.26 (m, 1H), 4.13 (d, J = 4.8 Hz, 2H), 3.42 (s, 3H) |

^{†}BRM K_{d} (nM)
A < 500
500 ≤ B < 2500
2500 ≤ C < 10,000
D ≥ 10,000

The contents of all references, patents, pending patent applications and published patents, cited throughout this application are hereby expressly incorporated by reference. Those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

What is claimed is:

1. A bifunctional compound having the chemical structure:
PTM-L-ULM,
or a pharmaceutically acceptable salt, enantiomer, stereoisomer, solvate, polymorph or prodrug thereof, wherein:
(a) the L is a chemical linking moiety connecting the ULM and the PTM, and comprises a chemical structural unit represented by the formula:
-(A^{L})_{q}-,
wherein:
(A^{L})_{q} is a group which is connected to the ULM and the PTM;
q is an integer greater than or equal to 1;
each A^{L} is independently selected from the group consisting of CR^{L1}R^{L2}, O, SO₂, NR^{L3}, CONR^{L3}, CO, CR^{L1}=CR^{L2}, C≡C, C₃₋₁₁cycloalkyl optionally substituted with 1-6 R^{L1} and/or R^{L2} groups, C₃₋₁₁heterocyclyl optionally substituted with 1-6 R^{L1} and/or R^{L2} groups, aryl optionally substituted with 1-6 R^{L1} and/or R^{L2} groups, and heteroaryl optionally substituted with 1-6 R^{L1} and/or R^{L2} groups, where R^{L1} or R^{L2}, each independently are optionally linked to other groups to form cycloalkyl and/or heterocyclyl moiety, optionally substituted with 1-4 R^{L5} groups; and
R^{L1}, R^{L2}, R^{L3}, and R^{L5} are, each independently, halogen, C₁₋₈alkyl, OC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₃₋₈cycloalkyl, NHC₃₋₈cycloalkyl, N(C₃₋₈cycloalkyl)₂, N(C₃₋₈cycloalkyl)(C₁₋₈alkyl), OH, NH₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, COC₁₋₈alkyl, CO₂H, halogen, CN, CF₃, CHF₂, CH₂F, NO₂, CONHC₁₋₈alkyl, or CON(C₁₋₈alkyl)₂;
(b) the ULM is a small molecule E3 ubiquitin ligase binding moiety that binds a Von Hippel-Lindau E3 ubiquitin ligase and is represented by the chemical structure: 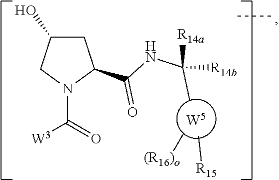
wherein:
W³ is selected from the group of an optionally substituted aryl, optionally substituted heteroaryl, or 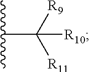
R₉ and R₁₀ are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl;
R₁₁ is selected from the group of an optionally substituted heterocyclyl, optionally substituted alkoxy, optionally substituted heteroaryl, optionally substituted aryl, 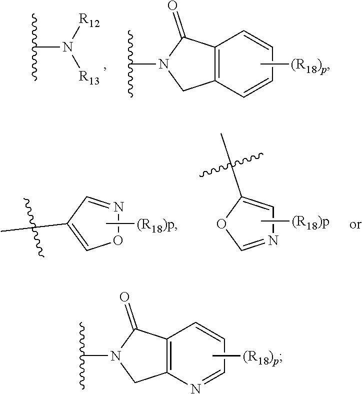
R₁₂ is selected from the group of H or optionally substituted alkyl;
R₁₃ is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₄ₐ and R_{14b} are each independently selected from H, amine, haloalkyl, optionally substituted alkyl, optionally substituted alkoxy, optionally substituted hydroxyl alkyl, optionally substituted alkylamine, optionally substituted amide, optionally substituted alkyl-amide, optionally substituted alkyl-cyano, optionally substituted alkyl-phosphate, optionally substituted heteroalkyl, optionally substituted alkyl-heterocycloalkyl, optionally substituted alkoxy-heterocycloalkyl, COR₂₆, alkyl-COR₂₆, CONR₂₇ₐR_{27b}, NHCOR₂₆, or NHCH₃COR₂₆, and the other of R₁₄ₐ and R_{14b} is H; or R₁₄ₐ, R_{14b}, together with the carbon atom to which they are attached, form an optionally substituted 3 to 5 membered cycloalkyl, heterocycloalkyl, spirocycloalkyl or spiroheterocyclyl, wherein the spiroheterocyclyl is not epoxide or aziridine;
W⁵ is optionally substituted phenyl, optionally substituted napthyl, or an optionally substituted 5-10 membered heteroaryl;
R₁₅ is selected from the group of H, halogen, CN, C≡CH, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐSO₂R_{27b}, optionally substituted alkyl, optionally substituted haloalkyl, optionally substituted haloalkoxy; optionally substituted aryl; optionally substituted heteroaryl; optionally substituted cycloalkyl; or optionally substituted heterocyclyl;
each R₁₆ is independently selected from the group of halo, CN, optionally substituted alkyl, optionally substituted alkylamine, optionally substituted haloalkyl, hydroxy, or optionally substituted haloalkoxy;
o is 0, 1, 2, 3, or 4;
R₁₈ is independently selected from the group of H, halo, optionally substituted alkoxy, cyano, optionally substituted alkyl, haloalkyl, or haloalkoxy;
each R₂₆ is independently selected from H, OH, optionally substituted alkyl or NR₂₇ₐR_{27b};
each R₂₇ₐ and R_{27b} is independently H, optionally substituted alkyl, optionally substituted cycloalkyl, or R₂₇ₐ and R_{27b} together with the nitrogen atom to which they are attached form a 4-6 membered heterocyclyl; and
p is 0, 1, 2, 3, or 4, and
the [symbol] of the ULM indicates the site of attachment of a chemical linking moiety coupling the PTM to the ULM; and
(c) the PTM is a SMARCA2 protein targeting moiety that has a chemical structure selected from:
(i) the chemical structure: 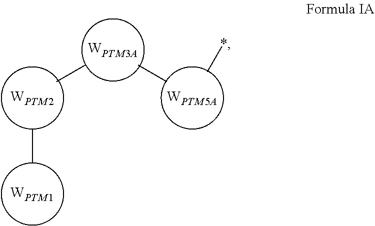
wherein:
W_{PTM1} is an optionally substituted 5-6-membered aryl or heteroaryl ring (e.g., a 5-6 member aryl or heteroaryl substituted with 0, 1, 2, or 3 substituents selected from hydroxy, halogen, alkoxy, alkyl, haloalkyl, amino, alkylamino and cyano, wherein the hydroxyl group of the phenyl is optionally substituted with a phosphate group);
W_{PTM2} is an optionally substituted 5-6-membered aryl or heteroaryl ring (e.g., a 5-6 membered aryl or heteroaryl substituted with 0, 1, 2, or 3 substituents selected from hydroxy, halogen, alkoxy, alkyl, haloalkyl, amino, alkylamino and cyano);
W_{PTM3A} is an optionally substituted 5-6-membered aryl or heteroaryl ring (e.g., a 5-6 membered aryl or heteroaryl substituted with 0, 1, 2, or 3 substituents selected from hydroxy, halogen, alkoxy, alkyl, haloalkyl, amino, alkylamino and cyano), or an optionally substituted 3-12 cycloalkyl or heterocyclyl (e.g. a 3-12 cycloalkyl or heterocyclyl substituted with 0, 1, or 2 substituents selected from hydroxy, halogen, alkoxy, alkyl, haloalkyl, amino, alkylamino and cyano), such as an optionally substituted bridged bicycloalkyl and bridged biheterocyclyl rings, optionally substituted 6-11 membered fused bicycloalkyl or fused biheterocycloalkyl rings (e.g. 6-11 membered fused bicycloalkyl or fused biheterocycloalkyl rings with 0, 1, or 2 substituents selected from hydroxy, halogen, alkoxy, alkyl, haloalkyl, amino, alkylamino and cyano), optionally substituted 6-12 membered spirocycloalkyl or spiroheterocyclyl rings (e.g. 6-12 member spirocycloalkyl or spiroheterocyclyl rings substituted with 0, 1, or 2 substituents selected from hydroxy, halogen, alkoxy, alkyl, haloalkyl, amino, alkylamino and cyano);
W_{PTM5A} is absent (such that W_{PTM3A} is connected directly to the chemical linking moiety (L)), an optionally substituted 5-6-membered aryl or heteroaryl ring (e.g. a 5-6 membered aryl or heteroaryl substituted with 0, 1, or 2 substituents selected from hydroxy, halogen, alkoxy, alkyl, haloalkyl, amino, alkylamino and cyano), or optionally substituted 3-7 cycloalkyl or heterocycloalkyl (e.g., optionally substituted 5-7 cycloalkyl or heterocyclyl, or a 3-7 or 5-7 cycloalkyl or heterocycloalkyl substituted with 0, 1, 2, or 3 substituents selected from hydroxy, halogen, alkoxy, alkyl, haloalkyl, amino, alkylamino and cyano); and [symbol]
is the attachment point to the chemical linking moiety,
wherein at least one of:
W_{PTM3A} is optionally substituted 6-11 membered fused bicycloalkyl rings, optionally substituted 6-11 membered fused biheterocycloalkyl rings, optionally substituted 6-12 membered spirocycloalkyl, or optionally substituted 6-12 membered spiroheterocyclyl rings (e.g. each optionally substituted with 0, 1, or 2 substituents selected from hydroxy, halogen, alkoxy, alkyl, haloalkyl, amino, alkylamino and cyano); or
W_{PTM5A} is an optionally substitute 3-7 cycloalkyl or heterocycloalkyl (e.g., optionally substituted 5-7 cycloalkyl or heterocycloalkyl or a 3-7 or 5-7 cycloalkyl or heterocycloalkyl substituted with 0, 1, 2, or 3 substituents selected from hydroxy, halogen, alkoxy, alkyl, haloalkyl, amino, alkylamino and cyano);
(ii) the chemical structure: 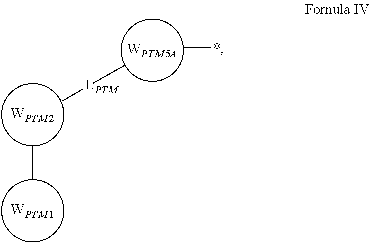
wherein
W_{PTM1} is an optionally substituted 5-6-membered aryl or heteroaryl ring (e.g., a 5-6 member aryl or heteroaryl substituted with 0, 1, 2, or 3 substituents selected from hydroxy, halogen, alkoxy, alkyl, haloalkyl, amino, alkylamino and cyano);
W_{PTM2} is an optionally substituted 5-6-membered aryl or heteroaryl ring (e.g., a 5-6 membered aryl or heteroaryl substituted with 0, 1, 2, or 3 substituents selected from hydroxy, halogen, alkoxy, alkyl, haloalkyl, amino, alkylamino and cyano);
W_{PTM5A} is an optionally substituted 3-7 cycloalkyl or heterocycloalkyl (e.g., optionally substituted 3-6 cycloalkyl or heterocycloalkyl, or a 3-7 or 3-6 cycloalkyl or heterocycloalkyl substituted with 0, 1, 2, or 3 substituents selected from hydroxy, halogen, alkoxy, alkyl, haloalkyl, amino, alkylamino and cyano);
L_{PTM} is selected from the group consisting of: a C₂₋₄ alkyne (e.g., a C₂₋₃ or C₂ alkyne) or a C₂₋₄ alkene (e.g., a C₂₋₃ or C₂ alkene) optionally substituted with 1-2 substituents independently selected from methyl, fluoro or haloalkyl; a C1-C2 alkyl optionally substituted with 1-2 substituents selected from methyl, fluoro, or haloalkyl; or a cyclopropyl optionally substituted with 1-2 substituents selected from methyl, fluoro, or haloalkyl; and [symbol]
is the attachment point to the chemical linking moiety; or
(iii) the chemical structure: 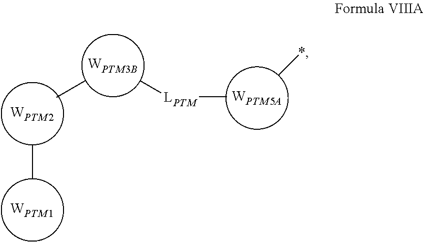
wherein:
W_{PTM1} is an optionally substituted 5-6-membered aryl or heteroaryl ring (e.g., a 5-6 member aryl or heteroaryl substituted with 0, 1, 2, or 3 substituents selected from hydroxy, halogen, alkoxy, alkyl, haloalkyl, amino, alkylamino and cyano);
W_{PTM2} is an optionally substituted 5-6-membered aryl or heteroaryl ring (e.g., a 5-6 membered aryl or heteroaryl substituted with 0, 1, 2, or 3 substituents selected from hydroxy, halogen, alkoxy, alkyl, haloalkyl, amino, alkylamino and cyano);
W_{PTM3B} is an optionally substituted 5-6-membered aryl or heteroaryl ring (e.g., a 5-6 membered aryl or heteroaryl substituted with 0, 1, 2, or 3 substituents selected from hydroxy, halogen, alkoxy, alkyl, haloalkyl, amino, alkylamino and cyano), or an optionally substituted 3-12 membered cycloalkyl or heterocycloalkyl (e.g. a 3-12 membered cycloalkyl or heterocycloalkyl substituted with 0, 1, or 2 substituents selected from hydroxy, halogen, alkoxy, alkyl, haloalkyl, amino, alkylamino and cyano), optionally substituted 3-10 membered fused bicycloalkyl and fused biheterocyclyl rings (e.g. 3-10 membered fused bicycloalkyl or fused biheterocyclyl rings substituted with 0, 1, or 2 substituents selected from hydroxy, halogen, alkoxy, alkyl, haloalkyl, amino, alkylamino and cyano), optionally substituted 3-12 membered bridged bicycloalkyl or bridged biheterocyclyl rings (e.g. a 3-12 membered bridged bicycloalkyl or bridged biheterocyclyl rings substituted with 0, 1, or 2 substituents selected from hydroxy, halogen, alkoxy, alkyl, haloalkyl, amino, alkylamino and cyano), optionally substituted 6-12 membered spirocycloalkyl or spiroheterocyclyl rings (e.g. a 6-12 member spirocycloalkyl or spiroheterocyclyl rings substituted with 0, 1, or 2 substituents selected from hydroxy, halogen, alkoxy, alkyl, haloalkyl, amino, alkylamino and cyano);
W_{PTM5A} is an optionally substituted 5-6-membered aryl or heteroaryl ring (e.g. a 5-6 membered aryl or heteroaryl substituted with 0, 1, or 2 substituents selected from hydroxy, halogen, alkoxy, alkyl, haloalkyl, amino, alkylamino and cyano), or an optionally substitute 3-7 cycloalkyl or heterocycloalkyl (e.g., optionally substituted 5-7 cycloalkyl or heterocycloalkyl or a 3-7 or 5-7 cycloalkyl or heterocycloalkyl substituted with 0, 1, 2, or 3 substituents selected from hydroxy, halogen, alkoxy, alkyl, haloalkyl, amino, alkylamino and cyano);
L_{PTM} is O or C1-C2 alkyl (e.g., a methylene group) optionally substituted with 0, 1, 2, or 3 groups selected from an C1-C4 alkyl (e.g., methyl or ethyl), C1-C3 alkoxy (e.g. methoxy or ethoxy), halogen (e.g., F or Cl), and =O; and [symbol]
is the attachment point to the chemical linking moiety,
wherein at least one of:
W_{PTM3B} is optionally substituted 3-10 fused bicycloalkyl rings, optionally substituted 3-10 fused biheterocyclyl rings, optionally substituted 6-12 membered spirocycloalkyl rings, or optionally substituted 6-12 membered spiroheterocyclyl rings (e.g. each is optionally substituted with 0, 1, or 2 substituents selected from hydroxy, halogen, alkoxy, alkyl, haloalkyl, amino, alkylamino and cyano); or
W_{PTM5A} is an optionally substituted 5-6-membered aryl or heteroaryl ring (e.g. a 5-6 membered aryl or heteroaryl substituted with 0, 1, or 2 substituents selected from hydroxy, halogen, alkoxy, alkyl, haloalkyl, amino, alkylamino and cyano); or
(iv) a chemical structure selected from: 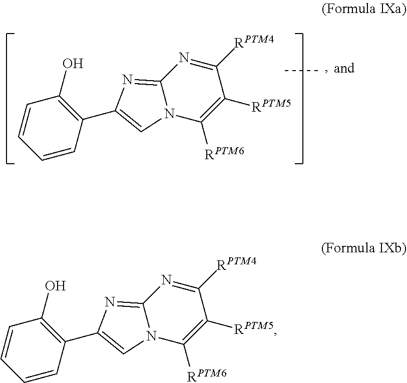
wherein:
the [symbol] of Formula IXa is the attachment point to the linker, ULM group, ULM′ group, VLM group, VLM′ group; and
the attachment point to the linker, ULM group, ULM′ group, VLM group, VLM′ group in Formula IXb is to the R^{PTM5} substitution or the R^{PTM6} substitution (e.g., the R^{PTM5} or the R^{PTM6} that is substituted) or at that location the R^{PTM5} location or the R^{PTM6} locations, such that the R^{PTM5} or the R^{PTM6} is a bond R^{PTM4} is selected from H, —NH₂, —OH, or —NR^{PTM7}R^{PTM8}, wherein R^{PTM7} and R^{PTM8} are independently selected C₁₋₄ alkyls; and
one of R^{PTM5} and R^{PTM6} is H or a bond when the point of attachment, and the other is selected from a H, a bond when the point of attachment, 5- or 6-membered aryl, 5- or 6-membered heteroaryl (e.g., 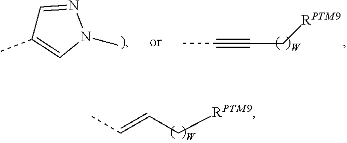
wherein W is 0, 1, or 2, and R^{PTM9} is selected from a H, alkyl (e.g., methyl), methoxy, optionally substituted 5- or 6-membered aryl, optionally substituted 5- or 6-membered heteroaryl (e.g., 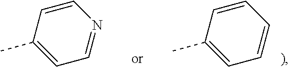
optionally substituted 4-6 membered cycloalkyl (e.g., optionally substituted 4- or 6-membered cycloalkyl), optionally substituted 4-6 heterocycloalkyl (e.g., optionally substituted 4- or 6-membered heterocycloalkyl), optionally substituted —O-4-6 membered cycloalkyl (e.g., —O-optionally substituted 4- or 6-membered cycloalkyl), optionally substituted —O-4-6 heterocycloalkyl (e.g., —O-optionally substituted 4- or 6-membered heterocycloalkyl), or C₁₋₆ alkylene group optionally interspaced with O atoms, provided that no heteroatom is directly attached to the double or triple carbon-carbon bond and any two heteroatoms are separated by at least two carbon atoms.

2. The compound according to claim 1, wherein the PTM has the chemical structure of Formula IA.

3. The compound according to claim 1, wherein the PTM has the chemical structure of Formula IV.

4. The compound according to claim 1, wherein the PTM has the chemical structure of Formula VIIIA.

5. The compound according to claim 1, wherein the PTM has the chemical structure of Formula IXa or IXb.

6. The compound according to any one of claims 1-5, wherein the PTM is selected from the group consisting of: 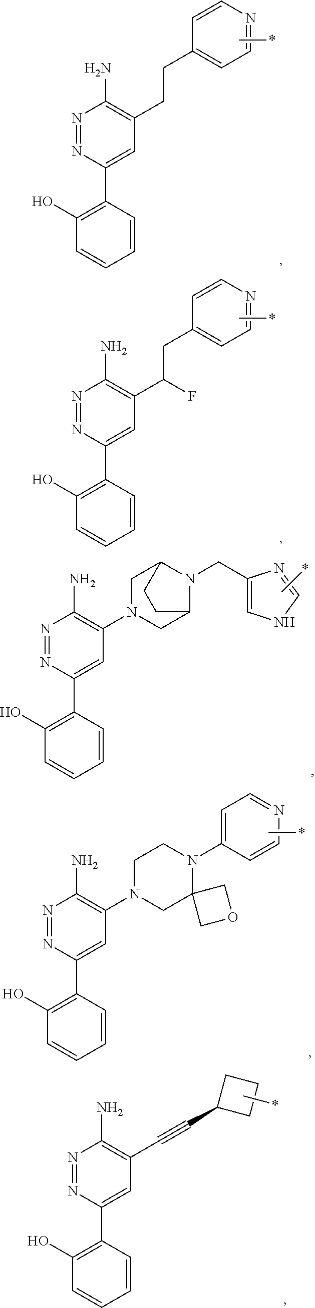 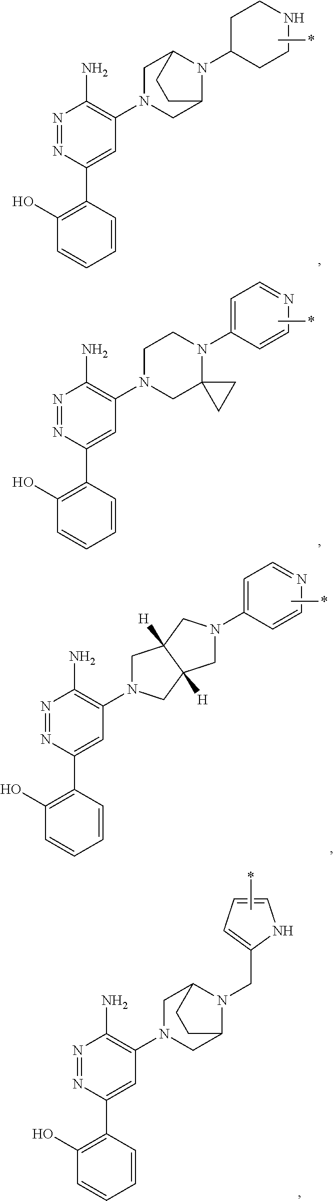 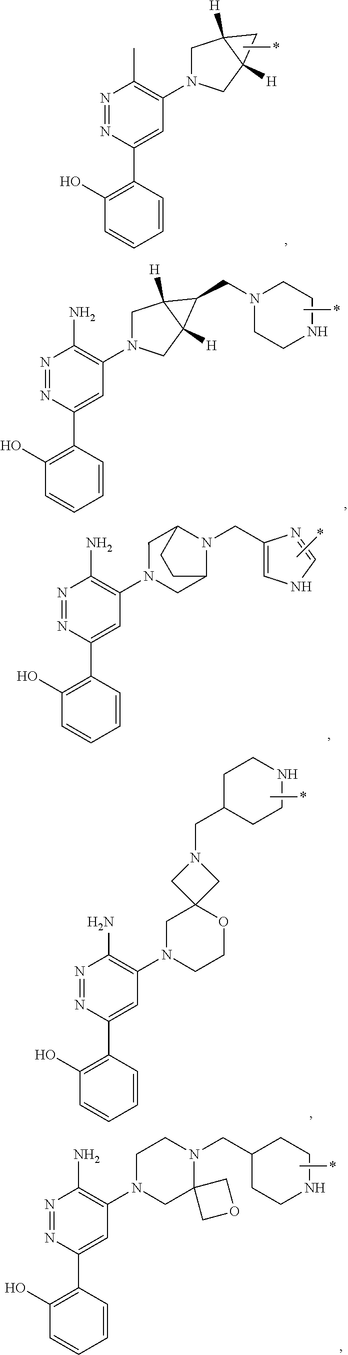 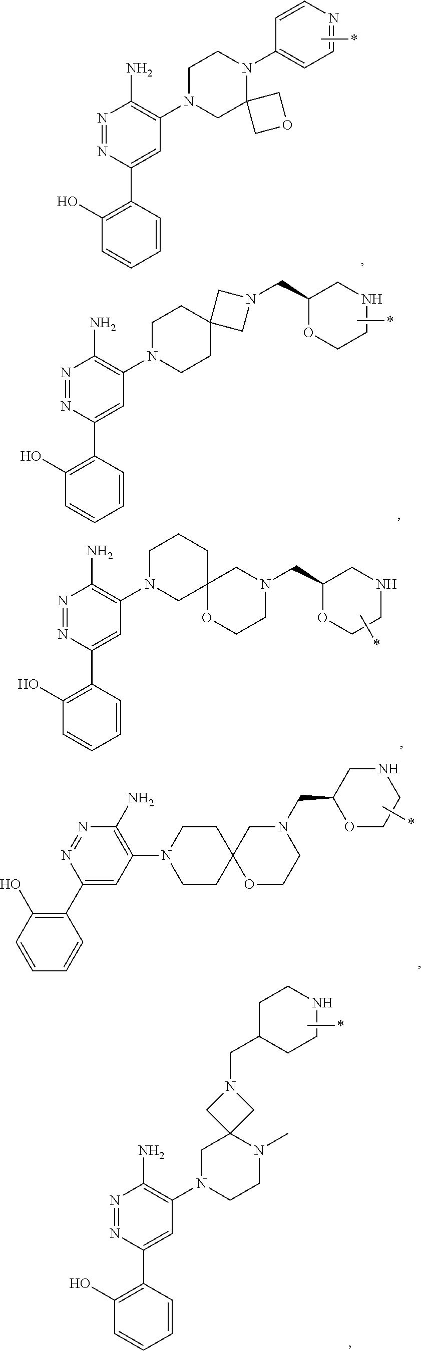 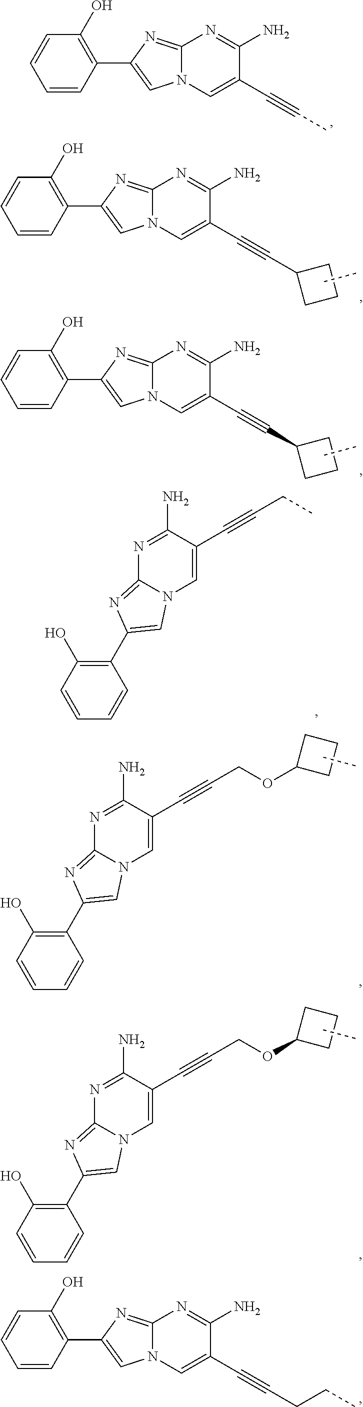 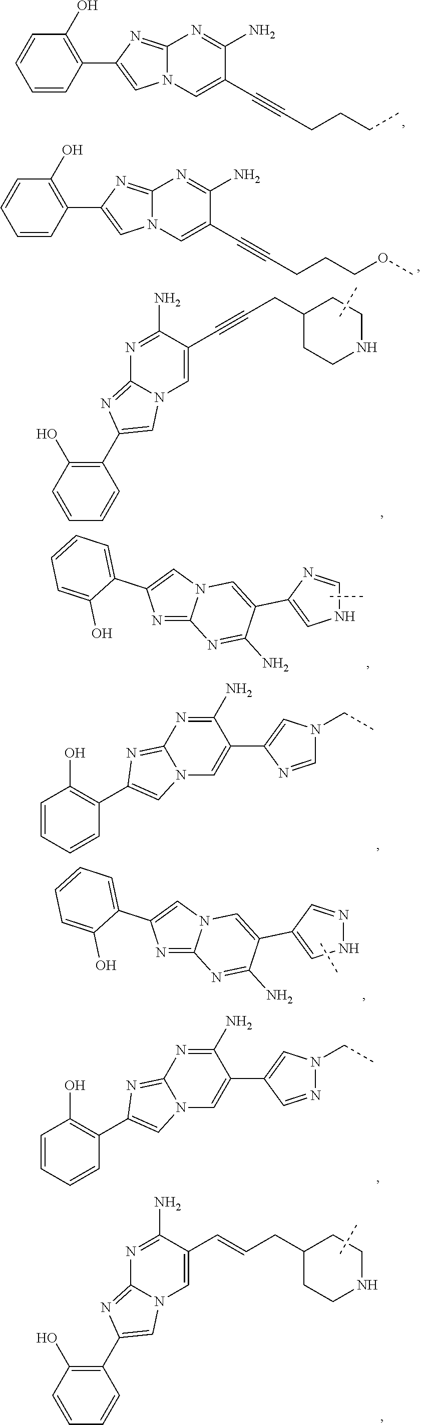 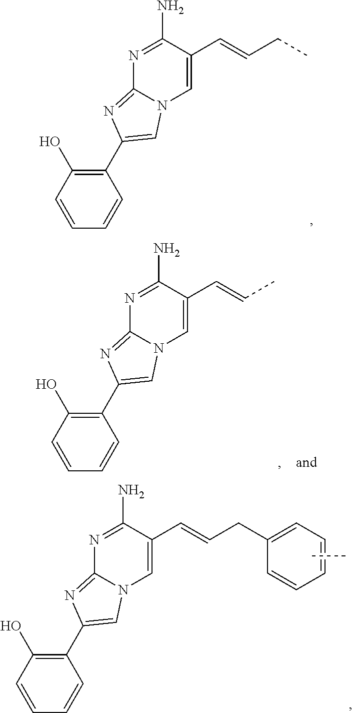
wherein [symbol] is the attachment point to the chemical linking moiety (e.g., the chemical linking moiety is attached to a carbon of the indicated ring or a nitrogen of the indicated non-aryl ring).

7. The compound according to any of claims 1-6 wherein the compound has a structure selected from: 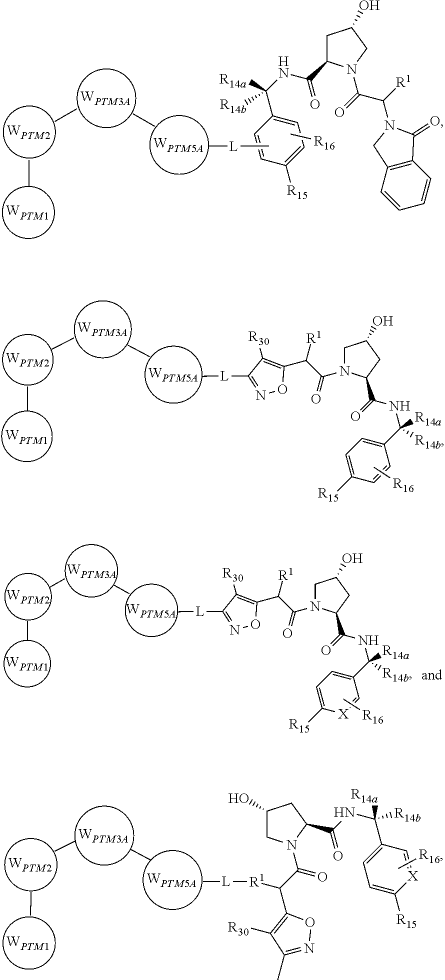
or a pharmaceutically acceptable salt thereof,
wherein:
X is CH or N;
R₃R is H, F or Cl;
R₁ is a C₁₋₆ alkyl; R₁₅ is selected from the group of H, halogen, CN, C≡CH, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐSO₂R_{27b}, optionally substituted alkyl, optionally substituted haloalkyl, optionally substituted haloalkoxy, optionally substituted aryl, optionally substituted heteroaryl, optionally substituted cycloalkyl, or optionally substituted heterocyclyl; and
each R₂₇ₐ and R_{27b} is independently H, optionally substituted alkyl, optionally substituted cycloalkyl, or R₂₇ₐ and R_{27b} together with the nitrogen atom to which they are attached form a 4-6 membered heterocyclyl.

8. The compound according to any of claims 1-6, wherein the compound has a structure selected from: 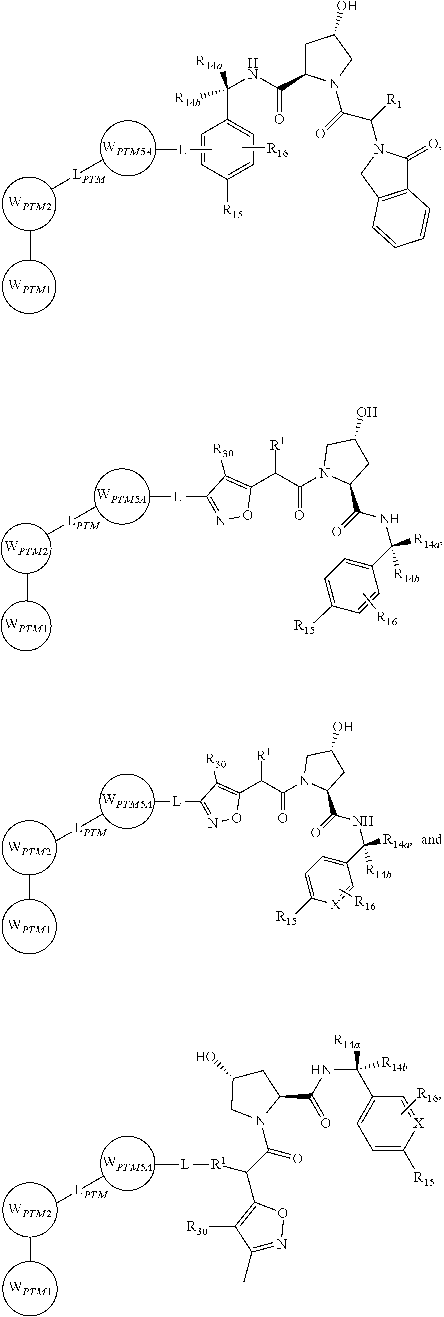
or a pharmaceutically acceptable salt thereof,
wherein:
X is CH or N;
R₃₀ is H, F or Cl;
R₁ is a C₁₋₆ alkyl;
R₁₅ is selected from the group of H, halogen, CN, C≡CH, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐSO₂R_{27b}, optionally substituted alkyl, optionally substituted haloalkyl, optionally substituted haloalkoxy, optionally substituted aryl, optionally substituted heteroaryl, optionally substituted cycloalkyl, or optionally substituted heterocyclyl; and
each R₂₇ₐ and R_{27b} is independently H, optionally substituted alkyl, optionally substituted cycloalkyl, or R₂₇ₐ and R_{27b} together with the nitrogen atom to which they are attached form a 4-6 membered heterocyclyl.

9. The compound according to any of claims 1-6, wherein the compound has a structure selected from: 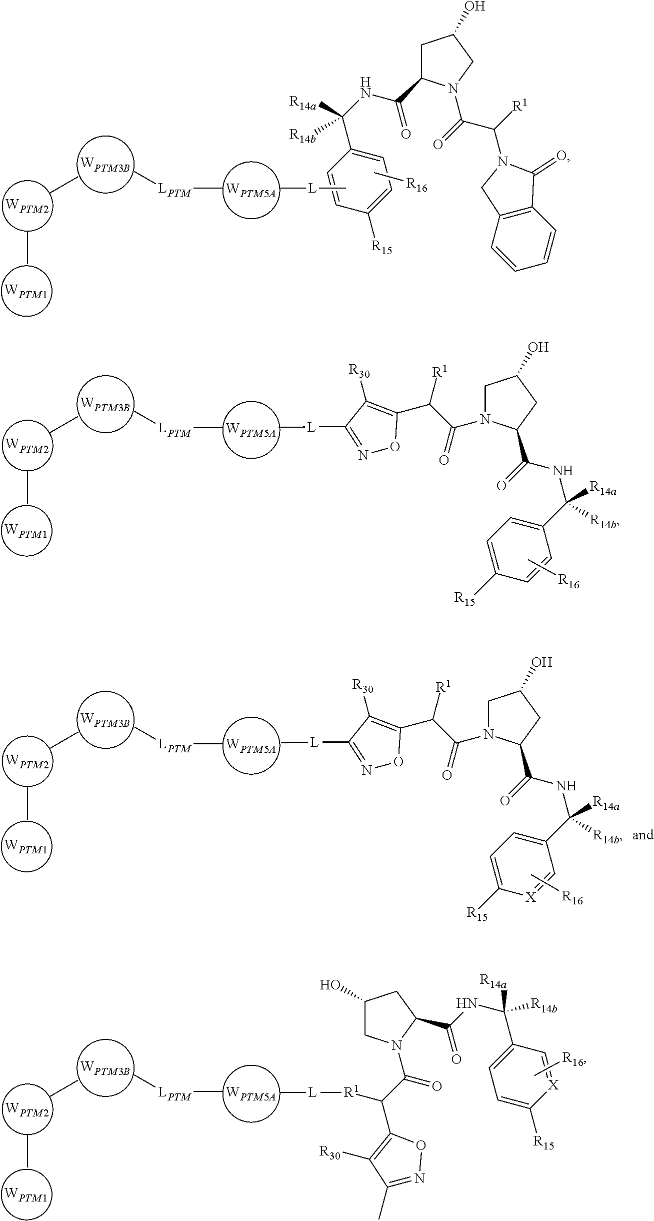
or a pharmaceutically acceptable salt thereof,
wherein:
X is CH or N;
R₃₀ is H, F or Cl;
R₁ is a C₁₋₆ alkyl;
R₁₅ is selected from the group of H, halogen, CN, C≡CH, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐSO₂R_{27b}, optionally substituted alkyl, optionally substituted haloalkyl, optionally substituted haloalkoxy, optionally substituted aryl, optionally substituted heteroaryl, optionally substituted cycloalkyl, or optionally substituted heterocyclyl; and
each R₂₇ₐ and R_{27b} is independently H, optionally substituted alkyl, optionally substituted cycloalkyl, or R₂₇ₐ and R_{27b} together with the nitrogen atom to which they are attached form a 4-6 membered heterocyclyl.

10. The compound according to any of claims 1-6, wherein the compound has a structure selected from: 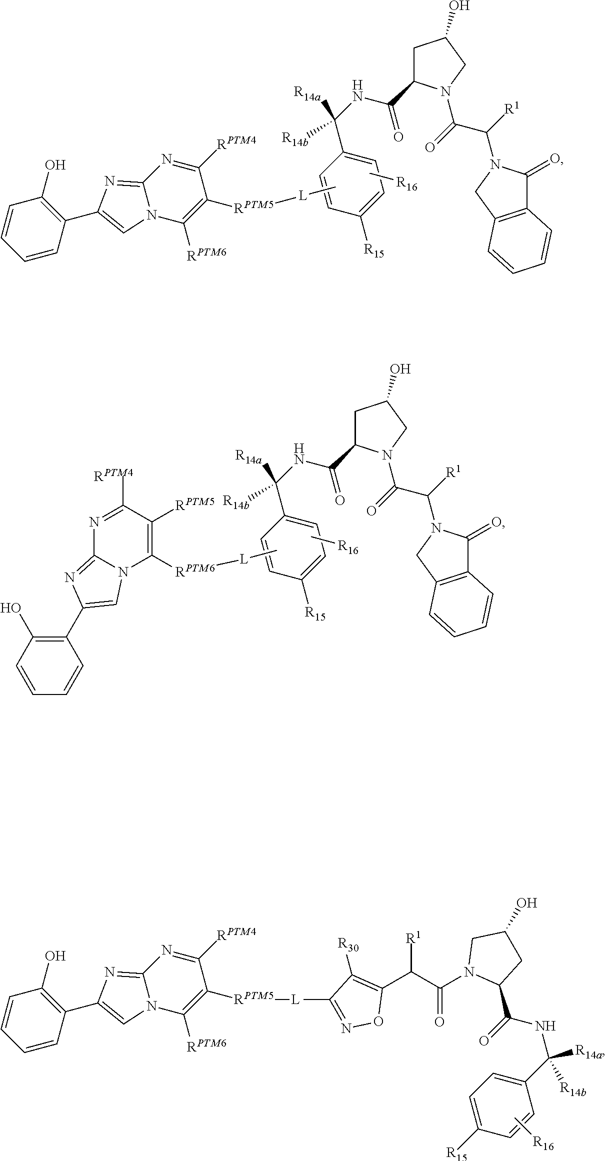 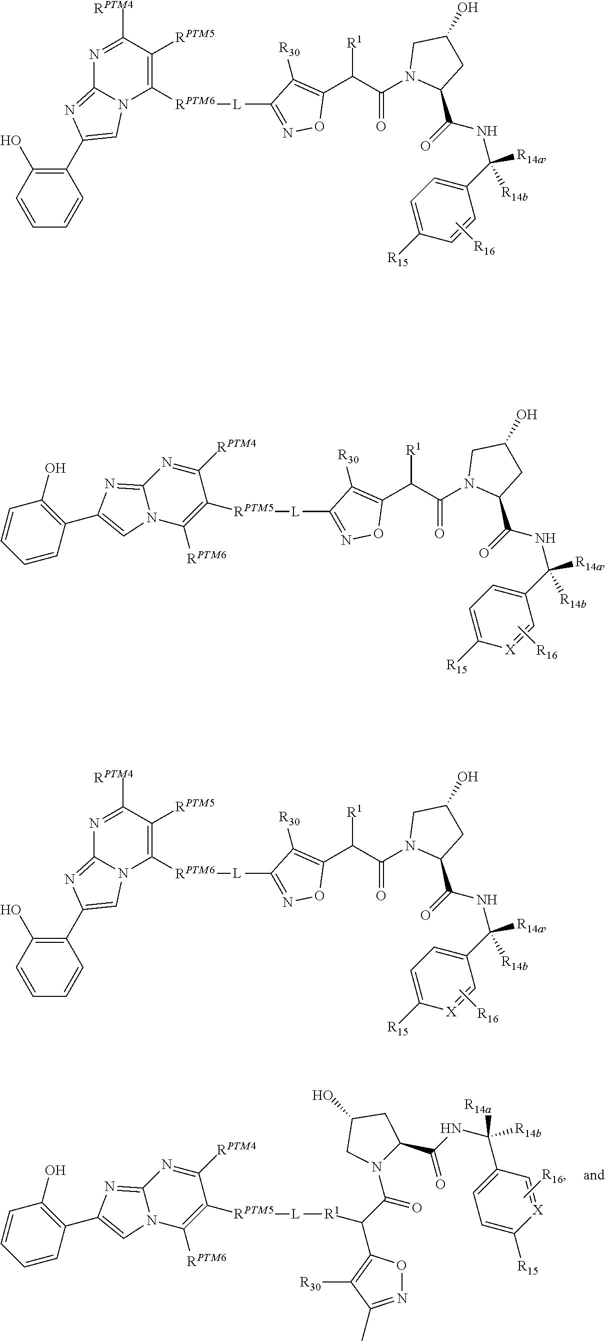 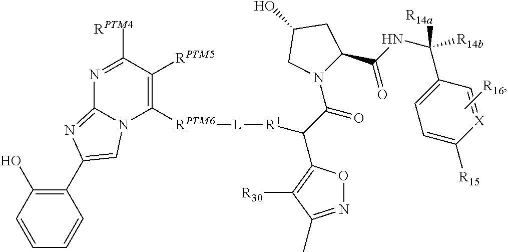
or a pharmaceutically acceptable salt thereof,
wherein:
X is CH or N;
R₃₀ is H, F or Cl;
R₁ is a C₁₋₆ alkyl; R₁₅ is selected from the group of H, halogen, CN, C≡CH, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐSO₂R_{27b}, optionally substituted alkyl, optionally substituted haloalkyl, optionally substituted haloalkoxy, optionally substituted aryl, optionally substituted heteroaryl, optionally substituted cycloalkyl, or optionally substituted heterocyclyl; and
each R₂₇ₐ and R_{27b} is independently H, optionally substituted alkyl, optionally substituted cycloalkyl, or R₂₇ₐ and R_{27b} together with the nitrogen atom to which they are attached form a 4-6 membered heterocyclyl.

11. The compound according to claim 7-10, wherein at least one of:
one of R₁₄ₐ and R_{14b} is a H, methyl, C₁ fluoroalkyl, CHF₂, CF₃, and the other is a H;
R₁₅ is selected from: cyano, halogen (e.g., F or Cl), 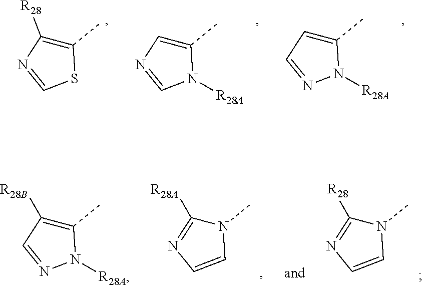
R¹⁶ is one or two groups individually selected from H, C₁₋₄alkyl, fluoro, chloro, NH₂, CN, or C₁₋₄alkoxy;
R_{28A} is selected from H or methyl;
R_{28B} is selected from H, methyl, and halogen (e.g., F or Cl); and
R₂₈ is H, methyl, CH₂N(Me)₂, CH₂OH, CH₂O(C₁₋₄alkyl), CH₂NHC(O)C₁₋₄alkyl, NH₂,

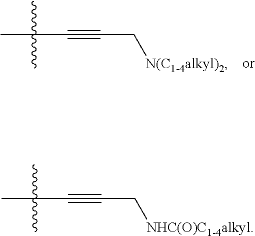

12. The compound according to any one of claims 1-6, wherein the ULM has a chemical structure selected from the group of: 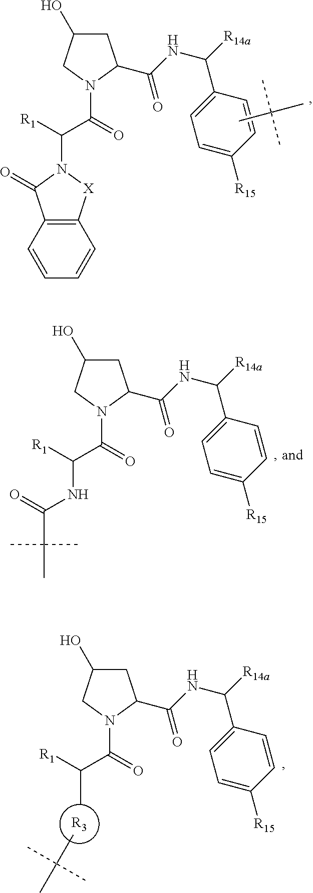
wherein:
R₁ is H, optionally substituted alkyl, optionally substituted cycloalkyl, optionally substituted hydroxyalkyl, optionally substituted heteroaryl, or haloalkyl;
R₁₄ₐ is H, haloalkyl, optionally substituted alkyl, methyl, fluoromethyl, hydroxymethyl, ethyl, isopropyl, or cyclopropyl;
R₁₅ is selected from the group consisting of H, halogen, CN, C≡CH, OH, NO₂, optionally substituted heteroaryl, optionally substituted aryl; optionally substituted alkyl, optionally substituted haloalkyl, optionally substituted alkoxy, optionally substituted haloalkoxy, optionally substituted cycloalkyl, or optionally substituted heterocyclyl;
X is C, CH₂, or C═O;
R₃ is absent or an optionally substituted 5 or 6 membered heteroaryl; and
the 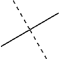
indicates the site of attachment of a chemical linking moiety coupling the PTM to the ULM.

13. The compound of any one of claims 1-6, wherein the ULM is of the formula: 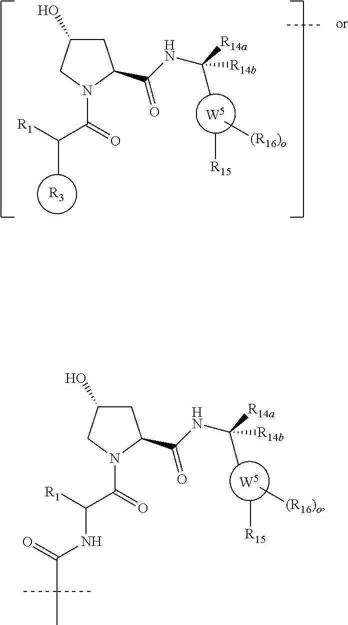
or a pharmaceutically acceptable salt thereof, wherein:
R₁ is H, optionally substituted alkyl or optionally substituted cycloalkyl;
R₃ is an optionally substituted 5-6 membered heteroaryl;
W⁵ is optionally substituted phenyl, optionally substituted napthyl or optionally substituted pyridinyl;
one of R₁₄ₐ and R_{14b} is H, optionally substituted alkyl, haloalkyl, optionally substituted alkoxy, optionally substituted hydroxyl alkyl, optionally substituted alkylamine, optionally substituted amide, optionally substituted alkyl-amide, optionally substituted alkyl-cyano, optionally substituted heteroalkyl, or optionally substituted alkyl-heterocycloalkyl, and the other of R₁₄ₐ and R_{14b} is H; or R₁₄ₐ, R_{14b}, together with the carbon atom to which they are attached, form an optionally substituted 3 to 5 membered cycloalkyl, heterocycloalkyl, spirocycloalkyl or spiroheterocyclyl, wherein the spiroheterocyclyl is not epoxide or aziridine;
R₁₅ is CN, C≡CH, fluoroalkyl, 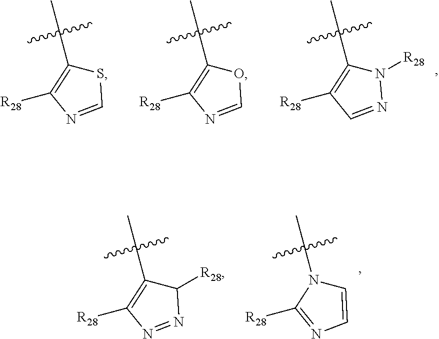
or optionally substituted 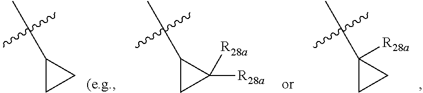
wherein R₂₈ₐ is halo, optionally substituted alkyl or fluoroalkyl);
each R₁₆ is independently selected from halo, CN, optionally substituted alkyl, optionally substituted haloalkyl, hydroxy, or haloalkoxy;
each R₂₇ₐ and R_{27b} is independently H, optionally substituted alkyl, optionally substituted 3-5 membered cycloalkyl, or R₂₇ₐ and R_{27b} together with the nitrogen atom to which they are attached form a 4-6 membered heterocyclyl;
R₂₈ is H, optionally substituted alkyl, optionally substituted alkoxy, optionally substituted heteroalkyl, optionally substituted alkylamine, optionally substituted hydroxyalkyl, amine, optionally substituted alkynyl, or optionally substituted cycloalkyl;
o is 0, 1 or 2; and
the 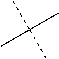
indicates the site of attachment of a chemical linking moiety coupling the PTM to the ULM.

14. The compound of claim 13, wherein the ULM is of the formula: 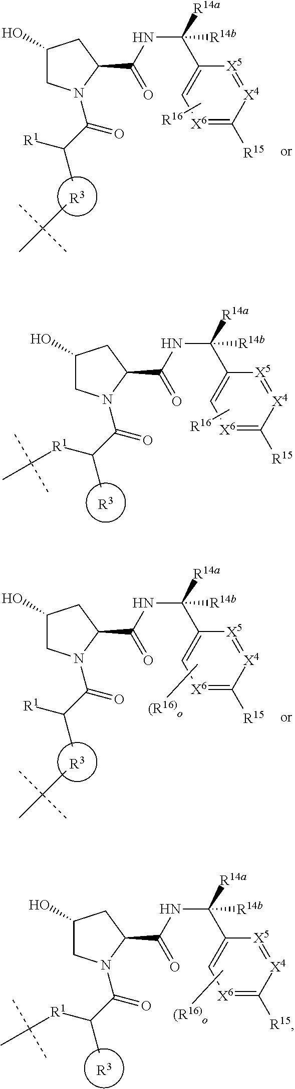
wherein:
o is 0, 1, or 2;
each of X⁴, X⁵, and X⁶ is selected from CH and N, wherein no more than 2 are N;
R¹ is C₁₋₆ alkyl;
one of R^{14a} and R^{14b} is H, optionally substituted alkyl, optionally substituted haloalkyl, optionally substituted alkoxy, optionally substituted hydroxyl alkyl, optionally substituted alkylamine, optionally substituted amide, optionally substituted alkyl-amide, optionally substituted alkyl-cyano, optionally substituted heteroalkyl, or optionally substituted alkyl-heterocycloalkyl, and the other of R^{14a} and R^{14b} is H; or R^{14a} and R^{14b}, together with the carbon atom to which they are attached, form an optionally substituted 3 to 5 membered cycloalkyl, heterocycloalkyl, spirocycloalkyl or spiroheterocyclyl, wherein the spiroheterocyclyl is not epoxide or aziridine;
each R₂₇ₐ and R_{27b} is independently H or C₁₋₆ alkyl or a 3-5 membered cycloalkyl;
R¹⁵ is, 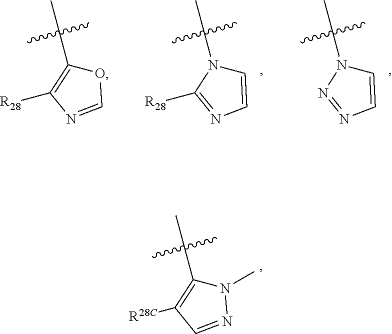
or CN;
R²⁸ is H, methyl, CH₂N(Me)₂, CH₂OH, CH₂O(C₁₋₄alkyl), CH₂NHC(O)C₁₋₄alkyl, NH₂, 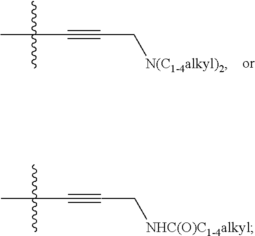
R^{28C} is H, methyl, fluoro, or chloro; and
R¹⁶ is H, C₁₋₄alkyl, fluoro, chloro, CN, or C₁₋₄alkoxy.

15. The compound of claim 13 or 14, wherein at least one of:
one R₁₄ₐ and R_{14b} are selected from: H, C₁₋₄ alkyl, C₃₋₄ cycloalkyl, C₁₋₄ haloalkyl, C₁₋₄ hydroxyalkyl, and C₁₋₄ alkyloxyalkyl;
one of R₁₄ₐ and R_{14b} is H; and
the 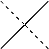
indicates the site of attachment of a chemical linking moiety coupling the PTM to the ULM.

16. The compound of claim 13, 14, or 15, wherein R³ is isoxazolyl, 4-chloroisoxazolyl, 4-fluoroisoxazolyl, or pyrazolyl.

17. The compound of claim 14, wherein the ULM is of the formula: 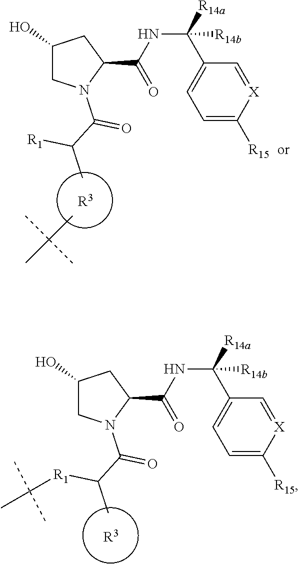
or a pharmaceutically acceptable salt thereof, wherein:
X is CH or N; and
one of R₁₄ₐ and R_{14b} is H, C₁₋₆ alkyl, C₁₋₆ haloalkyl, optionally substitute C₁₋₄ alkylamine, C₁₋₆ alkoxy, (CH₂)_{q}C₁₋₆ alkoxy, (CH₂)_{q}C₁₋₆ alkoxy-C₃₋₇ heterocycloalkyl, (CH₂)_{q}OH, (CH₂)_{q}NR₂₇ₐR_{27b}, C₃₋₆ cycloalkyl, or NR₂₇ₐR_{27b}; and one of R^{14a} and R^{14b} is H;
q is 1, 2, 3 or 4 (e.g., 1 or 2); and
the 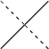
indicates the site of attachment of a chemical linking moiety coupling the PTM to the ULW.

18. The compound of claim 17, wherein R₁ is C₁₋₆ alkyl.

19. The compound of any one of claims 6-14, wherein:
one of R₁₄ₐ and R_{14b} is H, C₁₋₆ alkyl, C₁₋₆ haloalkyl, optionally substitute C₁₋₄ alkylamine, C₁₋₆ alkoxy, (CH₂)_{q}OH, (CH₂)_{q}NR₂₇ₐR_{27b}, C₃₋₆ cycloalkyl, or NR₂₇ₐR_{27b}; and one of R^{14a} and R^{14b} is H;
each R₂₇ₐ and R_{27b} is independently H or C₁₋₆ alkyl; and
q is 1, 2, 3 or 4.

20. The compound of any one of claims 6-14, wherein:
one of R₁₄ₐ and R_{14b} is H, C₁₋₄ alkyl, C₁₋₄ haloalkyl, C₁₋₄ alkoxy, optionally substituted C₁₋₄ alkylamine, (CH₂)_{q}C₁₋₆ alkoxy, (CH₂)_{q}OH, (CH₂)_{q}NR₂₇ₐR_{27b}, C₃₋₆ cycloalkyl, or NR₂₇ₐR_{27b}; and
one of R^{14a} and R_{14b} is H;
each R₂₇ₐ and R_{27b} is independently H or C₁₋₄ alkyl; and
q is 1 or 2.

21. The compound of any one of claims 10-20, wherein:
R₂₈ is C₁₋₆ alkyl, C₃₋₆ cycloalkyl, C₁₋₆ haloalkyl, (CH₂)_{q}OC₁₋₆alkyl, (CH₂)_{q}OH, (CH₂)_{q}NR₂₇ₐR_{27b}, (CH₂)_{q}NHCOC₁₋₆ alkyl, or 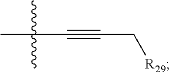
R₂₉ is H, C₁₋₆ alkyl, NR₂₇ₐR_{27b} or _{q}NHCOC₁₋₆ alkyl; and
q is 1 or 2.

22. The compound of claim 21, or a pharmaceutically acceptable salt thereof, wherein X is CH.

23. The compound of any one of claims 1-6, wherein the ULM has the formula: 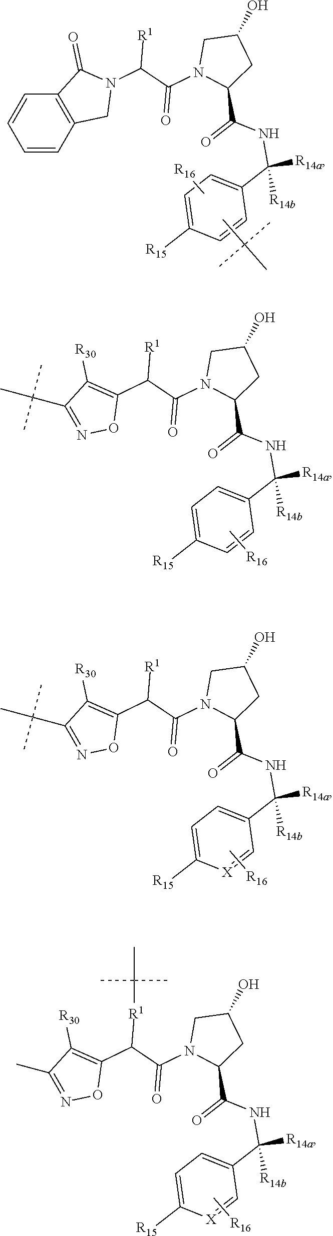
or a pharmaceutically acceptable salt thereof,
wherein:
X is CH or N;
R₃₀ is H, F or Cl;
R₁ is a C₁₋₆ alkyl;
one of R₁₄ₐ and R_{14b} is a H, methyl, C₁ fluoroalkyl, CHF₂, CF₃, and the other is a H;
R₁₅ is selected from: cyano, halogen (e.g., F or Cl), 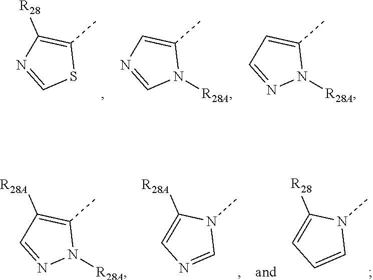
R¹⁶ is one or two groups individually selected from H, C₁₋₄alkyl, fluoro, chloro, NH₂, CN, or C₁₋₄alkoxy; and
R_{28A} is selected from H or methyl;
R_{28B} is selected from H, methyl, and halogen (e.g., F or Cl);
R₂₈ is H, methyl, CH₂N(Me)₂, CH₂OH, CH₂O(C₁₋₄alkyl), CH₂NHC(O)C₁₋₄alkyl, NH₂, 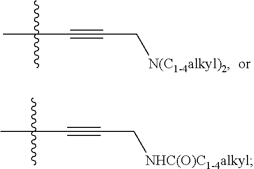
and
the 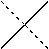
indicates the site of attachment of a chemical linking moiety coupling the PTM to the ULM.

24. The compound of any of claims 1-13, wherein one of R₁₄ₐ and R_{14b} is a H, C₁₋₃ alkyl (e.g., methyl), Cl fluoroalkyl, CHF₂, or CF₃, and the other is a H.

25. The compound of any of claims 1-6, wherein the ULM is selected from:

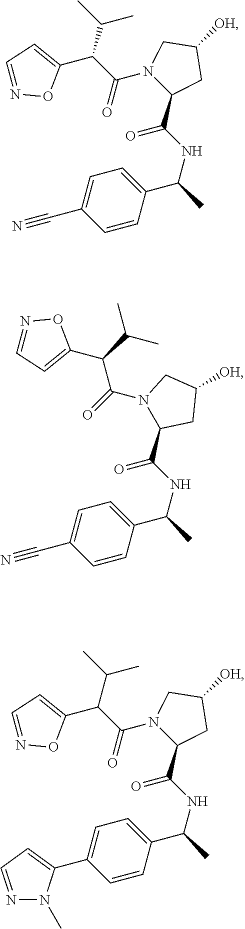 [SMILES: CC(C)[C@H](C(=O)N1C[C@H](O)C[C@H]1C(=O)N[C@@H](C)c1ccc(C#N)cc1)c1ccno1 ; CC(C)[C@@H](C(=O)N1C[C@H](O)C[C@H]1C(=O)N[C@@H](C)c1ccc(C#N)cc1)c1ccno1 ; CC(C)C(C(=O)N1C[C@H](O)C[C@H]1C(=O)N[C@@H](C)c1ccc(-c2ccnn2C)cc1)c1ccno1]

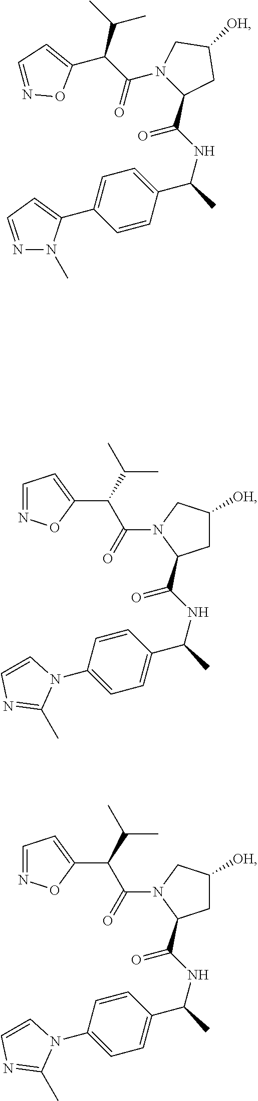 [SMILES: CC(C)[C@@H](C(=O)N1C[C@H](O)C[C@H]1C(=O)N[C@@H](C)c1ccc(-c2ccnn2C)cc1)c1ccno1 ; Cc1nccn1-c1ccc([C@H](C)NC(=O)[C@@H]2C[C@@H](O)CN2C(=O)[C@H](c2ccno2)C(C)C)cc1 ; Cc1nccn1-c1ccc([C@H](C)NC(=O)[C@@H]2C[C@@H](O)CN2C(=O)[C@@H](c2ccno2)C(C)C)cc1]

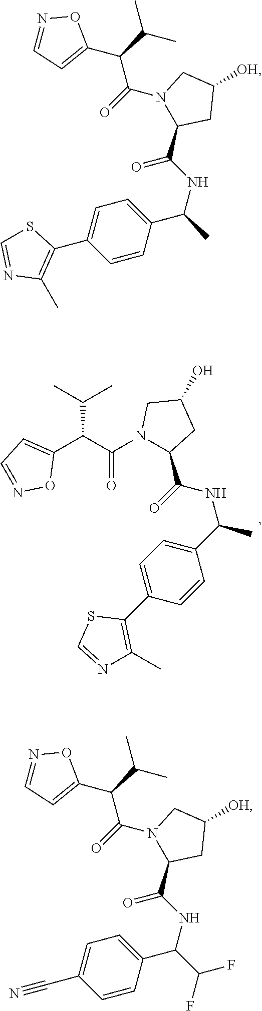 [SMILES: Cc1ncsc1-c1ccc([C@H](C)NC(=O)[C@@H]2C[C@@H](O)CN2C(=O)[C@@H](c2ccno2)C(C)C)cc1 ; Cc1ncsc1-c1ccc([C@H](C)NC(=O)[C@@H]2C[C@@H](O)CN2C(=O)[C@H](c2ccno2)C(C)C)cc1 ; CC(C)[C@@H](C(=O)N1C[C@H](O)C[C@H]1C(=O)NC(c1ccc(C#N)cc1)C(F)F)c1ccno1]

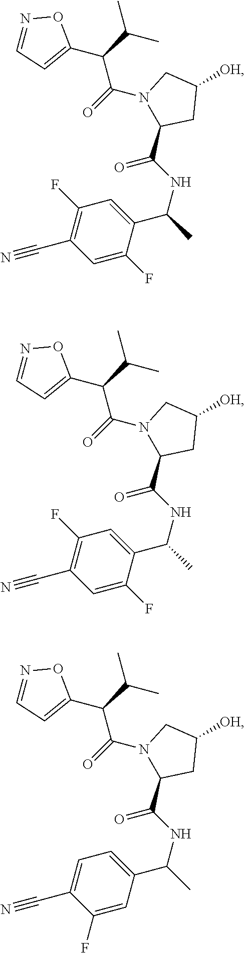 [SMILES: CC(C)[C@@H](C(=O)N1C[C@H](O)C[C@H]1C(=O)N[C@@H](C)c1cc(F)c(C#N)cc1F)c1ccno1 ; CC(C)[C@@H](C(=O)N1C[C@H](O)C[C@H]1C(=O)N[C@H](C)c1cc(F)c(C#N)cc1F)c1ccno1 ; CC(NC(=O)[C@@H]1C[C@@H](O)CN1C(=O)[C@@H](c1ccno1)C(C)C)c1ccc(C#N)c(F)c1]

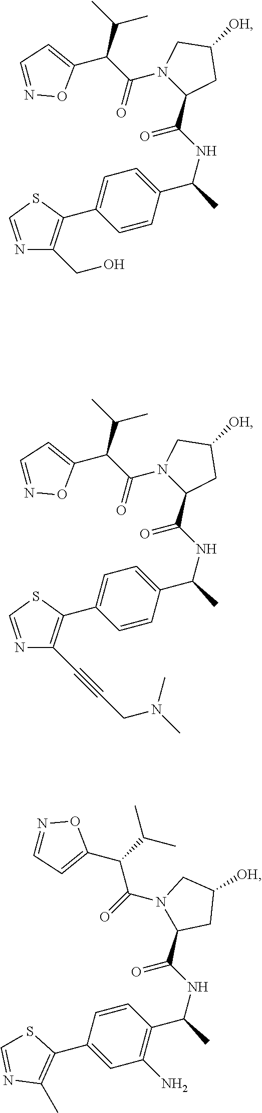 [SMILES: CC(C)[C@@H](C(=O)N1C[C@H](O)C[C@H]1C(=O)N[C@@H](C)c1ccc(-c2scnc2CO)cc1)c1ccno1 ; CC(C)[C@@H](C(=O)N1C[C@H](O)C[C@H]1C(=O)N[C@@H](C)c1ccc(-c2scnc2C#CCN(C)C)cc1)c1ccno1 ; Cc1ncsc1-c1ccc([C@H](C)NC(=O)[C@@H]2C[C@@H](O)CN2C(=O)[C@H](c2ccno2)C(C)C)c(N)c1]

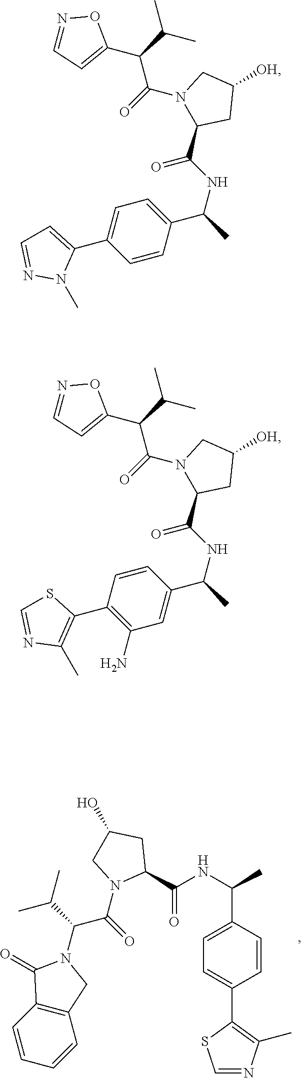 [SMILES: CC(C)[C@@H](C(=O)N1C[C@H](O)C[C@H]1C(=O)N[C@@H](C)c1ccc(-c2ccnn2C)cc1)c1ccno1 ; Cc1ncsc1-c1ccc([C@H](C)NC(=O)[C@@H]2C[C@@H](O)CN2C(=O)[C@@H](c2ccno2)C(C)C)cc1N ; Cc1ncsc1-c1ccc([C@H](C)NC(=O)[C@@H]2C[C@@H](O)CN2C(=O)[C@@H](C(C)C)N2Cc3ccccc3C2=O)cc1]

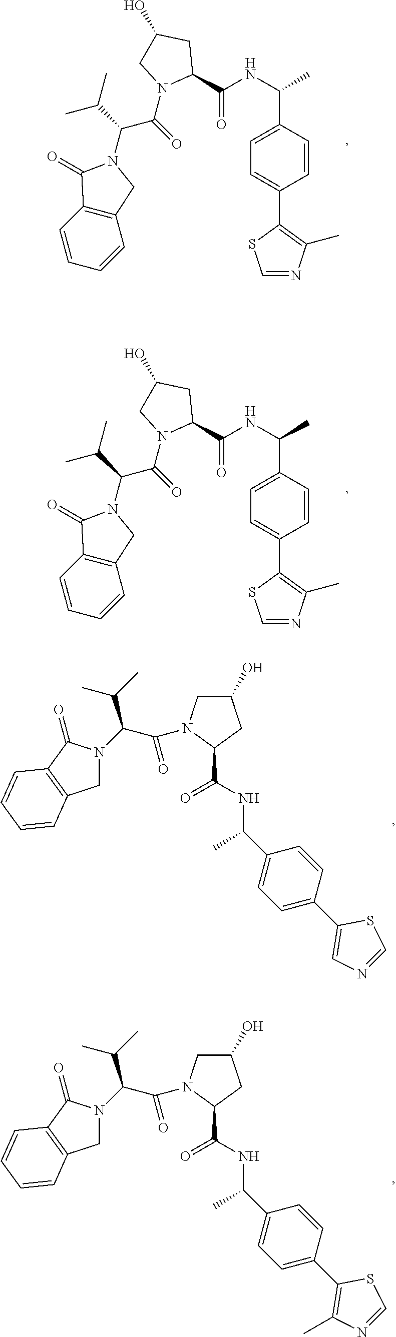

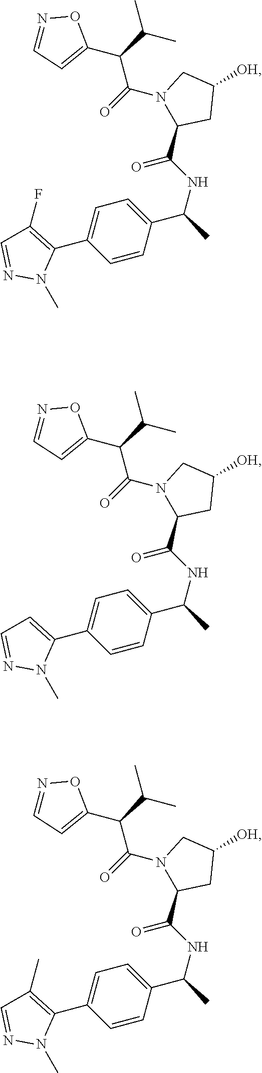 [SMILES: CC(C)[C@@H](C(=O)N1C[C@H](O)C[C@H]1C(=O)N[C@@H](C)c1ccc(-c2c(F)cnn2C)cc1)c1ccno1 ; CC(C)[C@@H](C(=O)N1C[C@H](O)C[C@H]1C(=O)N[C@@H](C)c1ccc(-c2ccnn2C)cc1)c1ccno1 ; Cc1cnn(C)c1-c1ccc([C@H](C)NC(=O)[C@@H]2C[C@@H](O)CN2C(=O)[C@@H](c2ccno2)C(C)C)cc1]

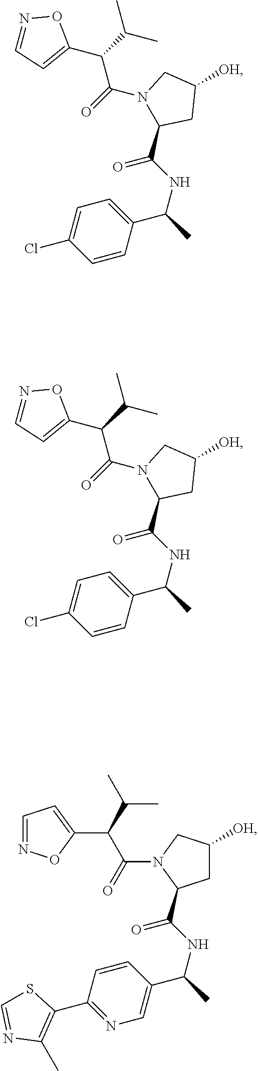 [SMILES: CC(C)[C@H](C(=O)N1C[C@H](O)C[C@H]1C(=O)N[C@@H](C)c1ccc(Cl)cc1)c1ccno1 ; CC(C)[C@@H](C(=O)N1C[C@H](O)C[C@H]1C(=O)N[C@@H](C)c1ccc(Cl)cc1)c1ccno1 ; Cc1ncsc1-c1ccc([C@H](C)NC(=O)[C@@H]2C[C@@H](O)CN2C(=O)[C@@H](c2ccno2)C(C)C)cn1]

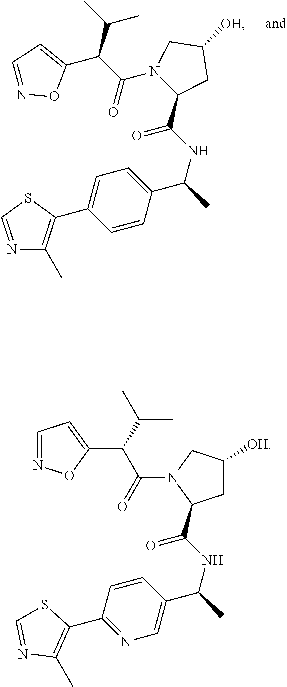 [SMILES: Cc1ncsc1-c1ccc([C@H](C)NC(=O)[C@@H]2C[C@@H](O)CN2C(=O)[C@@H](c2ccno2)C(C)C)cc1 ; Cc1ncsc1-c1ccc([C@H](C)NC(=O)[C@@H]2C[C@@H](O)CN2C(=O)[C@H](c2ccno2)C(C)C)cn1]

26. The compound of any one of claim 1-6, wherein the ULM is selected from:

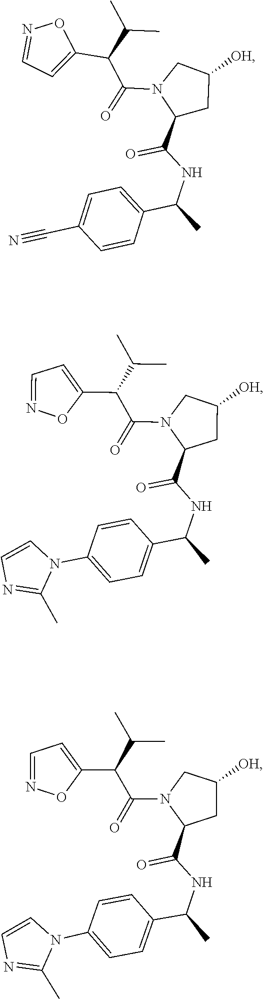 [SMILES: CC(C)[C@@H](C(=O)N1C[C@H](O)C[C@H]1C(=O)N[C@@H](C)c1ccc(C#N)cc1)c1ccno1 ; Cc1nccn1-c1ccc([C@H](C)NC(=O)[C@@H]2C[C@@H](O)CN2C(=O)[C@H](c2ccno2)C(C)C)cc1 ; Cc1nccn1-c1ccc([C@H](C)NC(=O)[C@@H]2C[C@@H](O)CN2C(=O)[C@@H](c2ccno2)C(C)C)cc1]

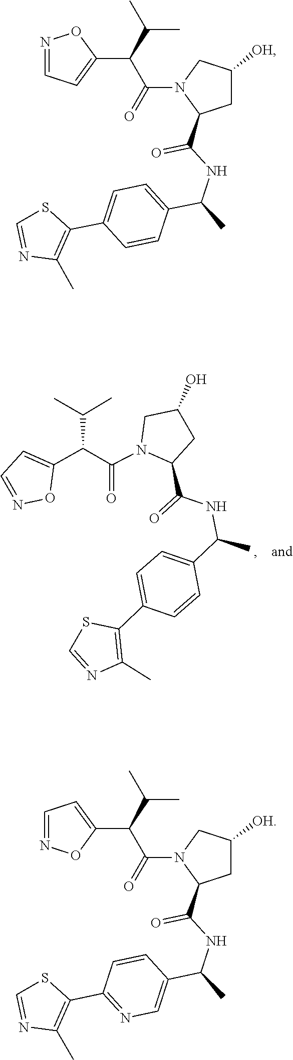 [SMILES: Cc1ncsc1-c1ccc([C@H](C)NC(=O)[C@@H]2C[C@@H](O)CN2C(=O)[C@@H](c2ccno2)C(C)C)cc1 ; Cc1ncsc1-c1ccc([C@H](C)NC(=O)[C@@H]2C[C@@H](O)CN2C(=O)[C@H](c2ccno2)C(C)C)cc1 ; Cc1ncsc1-c1ccc([C@H](C)NC(=O)[C@@H]2C[C@@H](O)CN2C(=O)[C@@H](c2ccno2)C(C)C)cn1]

27. The compound according to any of claims 1-26, wherein the chemical linking moiety (L) is selected from the group consisting of: 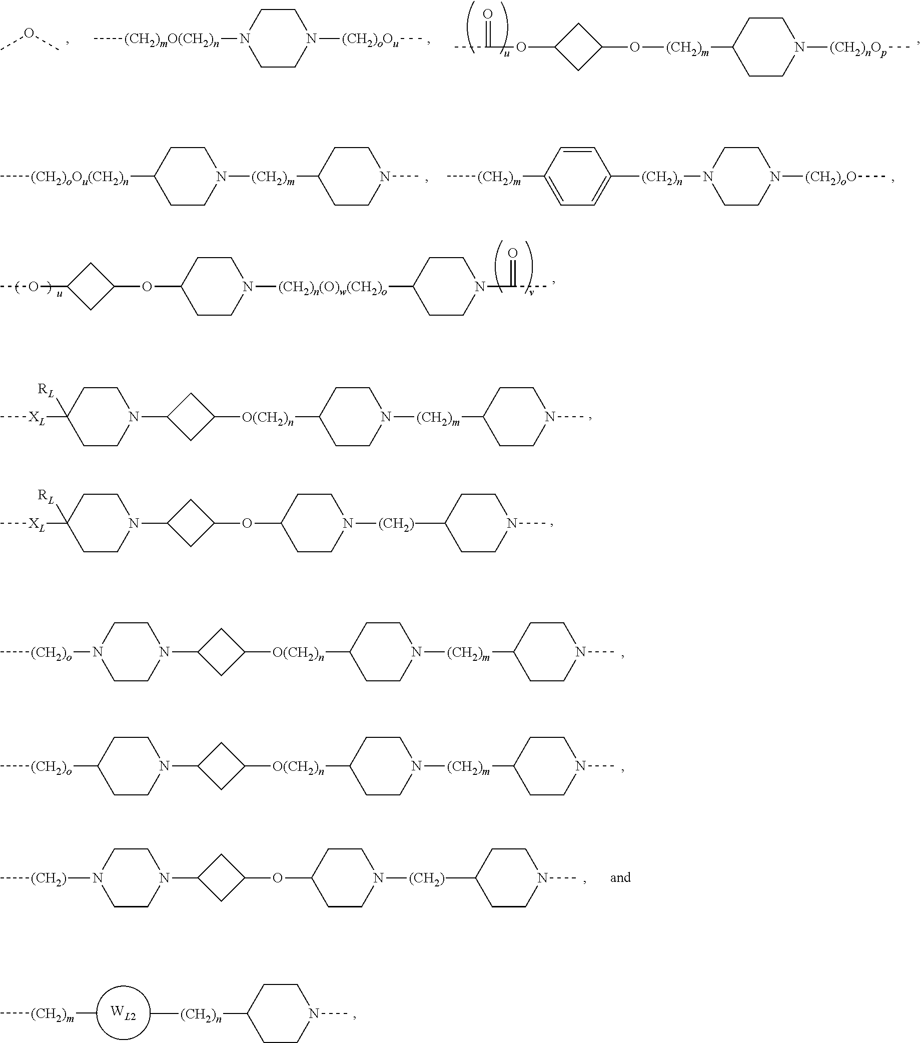
wherein:
each m, n, o, and p of the chemical linking moiety is independently selected from the integers 0, 1, 2, 3 and 4 (preferably 0, 1, or 2);
each u, w, and v of the chemical liking moiety is independently selected from integers 0 and 1;
X_{L} is —C(CH₂)—, —C(CH₃)H,— —CH₂—, —O—, C═O, or —NH—CH₂—;
R_{L} is H, OH, F, Cl, or methyl; and
W_{L2} is selected from an optionally substituted 6-12 membered spirocycloalkyl or spiroheterocyclyl (e.g. a 6-12 or 8-12 member spirocycloalkyl or spiroheterocyclyl substituted with 0, 1, or 2 substituents selected from hydroxy, halogen, C₁₋₃ alkoxy, C₁₋₃ alkyl, C₁₋₃ haloalkyl, or amino), and

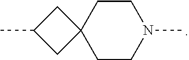

28. The compound according to claim 27, wherein each m, n, o, p, and q of the chemical linking moiety independently selected from the integers 0, 1, or 2.

29. The compound according to any one of claims 1-28, wherein the chemical linking moiety (L) is selected from the group consisting of:

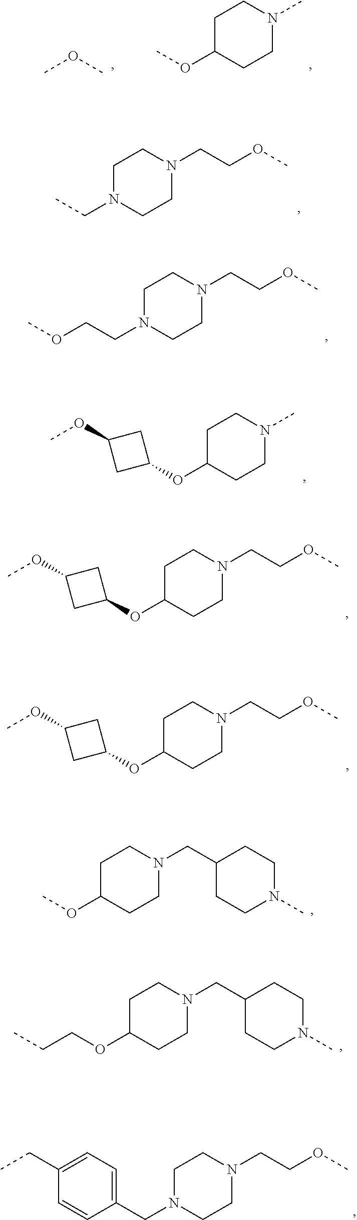

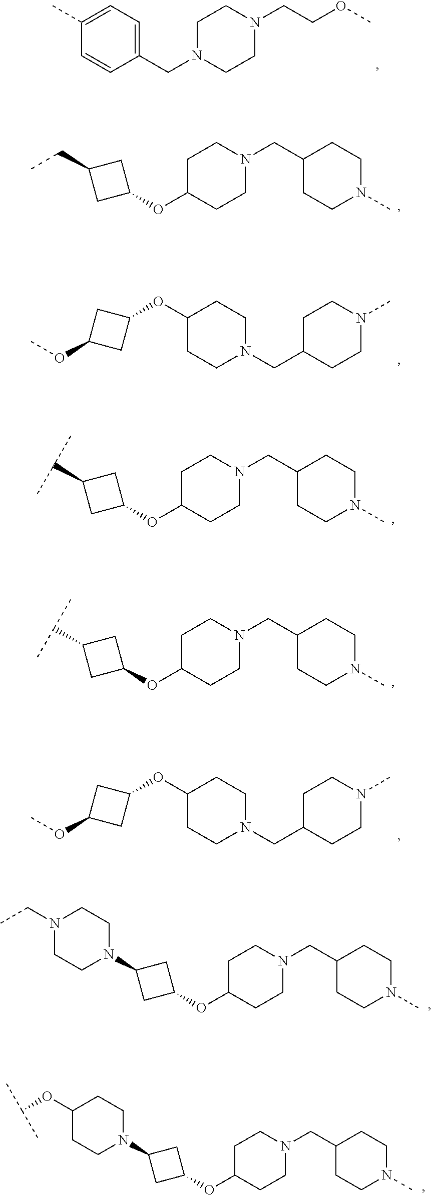

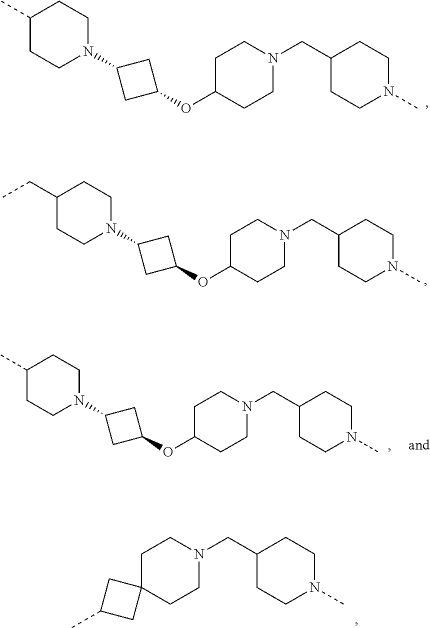

30. A compound selected from: 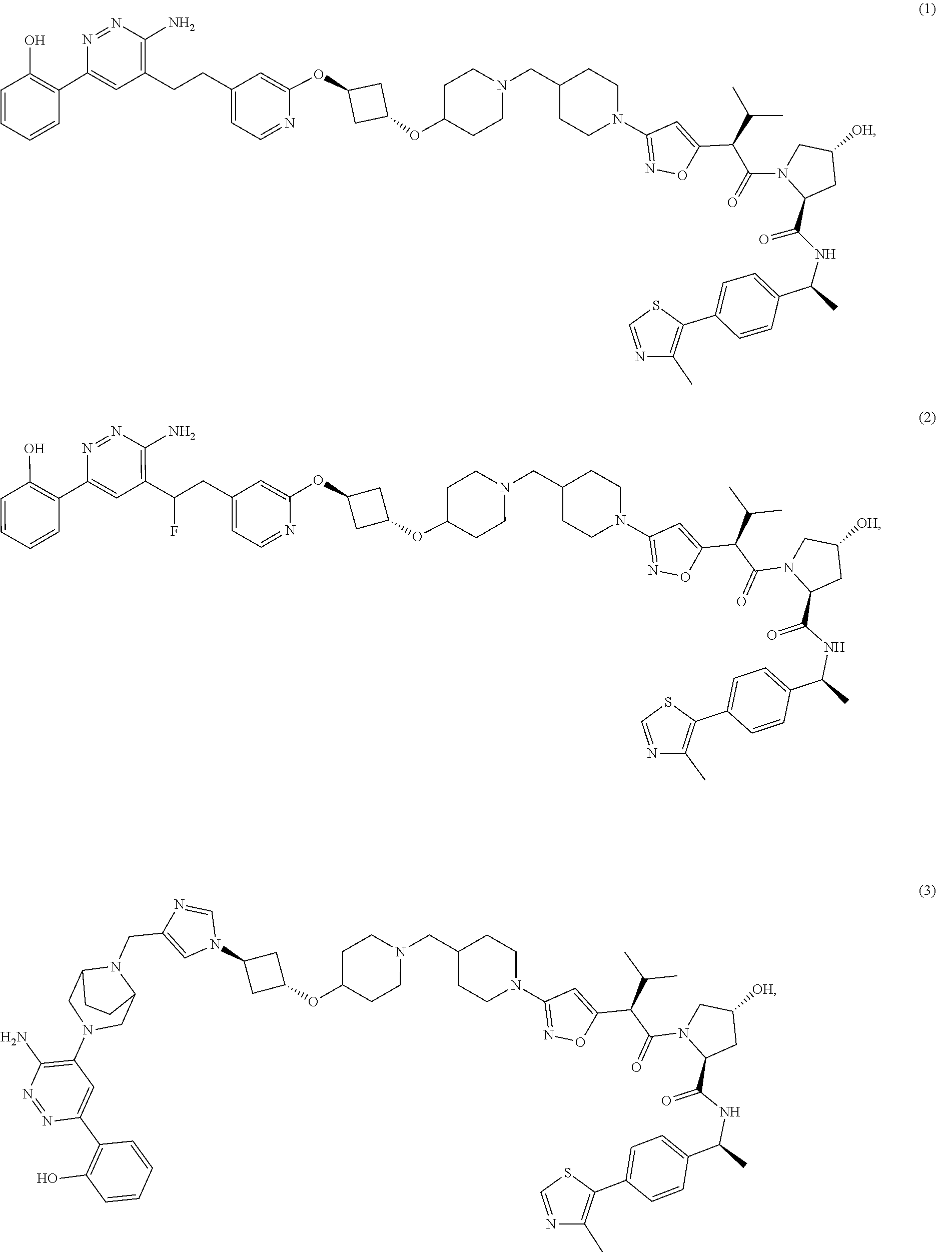 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Oc6cc(CCc7cc(-c8ccccc8O)nnc7N)ccn6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Oc6cc(CC(F)c7cc(-c8ccccc8O)nnc7N)ccn6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](n6cnc(CN7C8CCC7CN(c7cc(-c9ccccc9O)nnc7N)C8)c6)C5)CC4)CC3)no2)C(C)C)cc1] 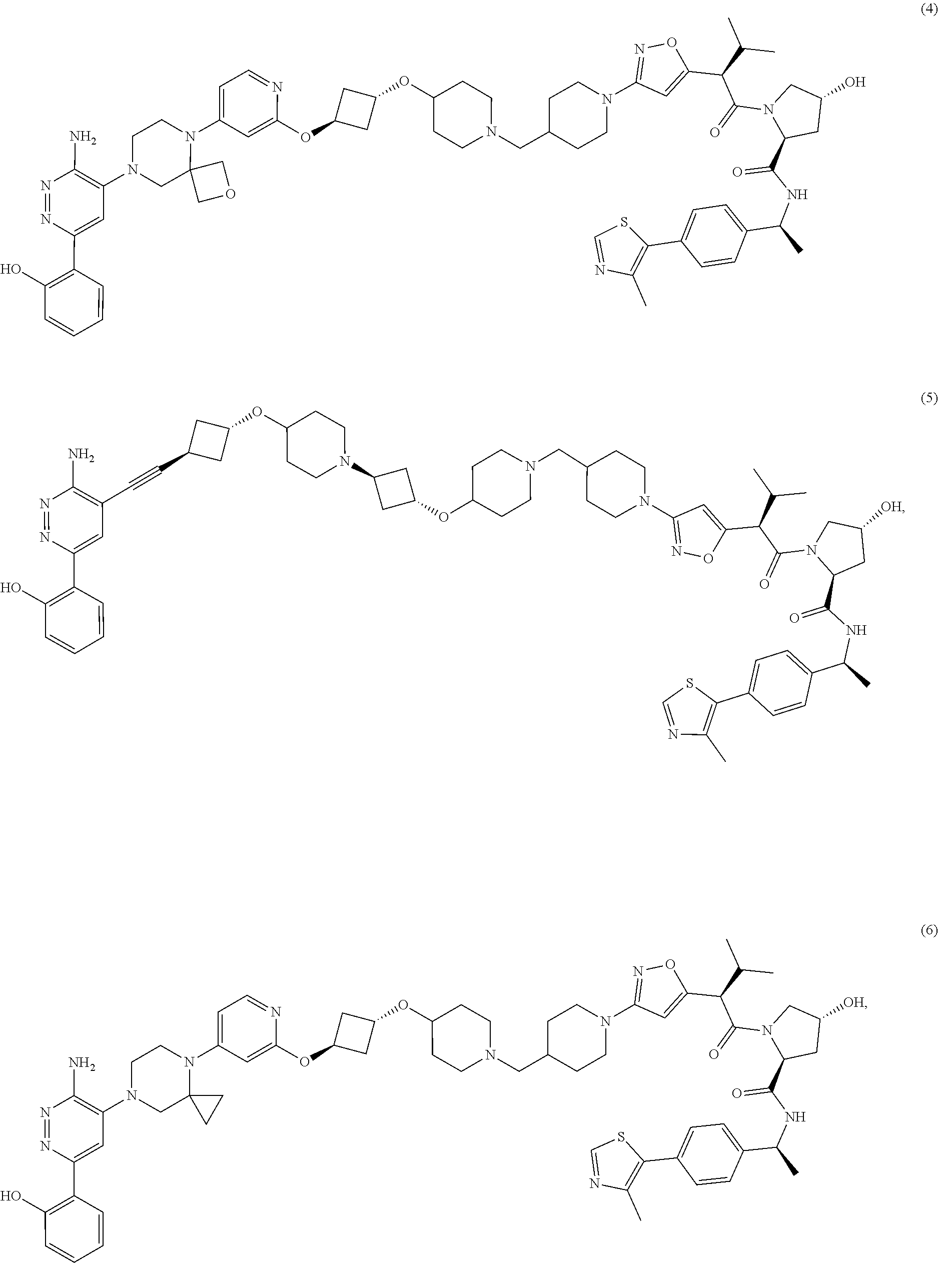 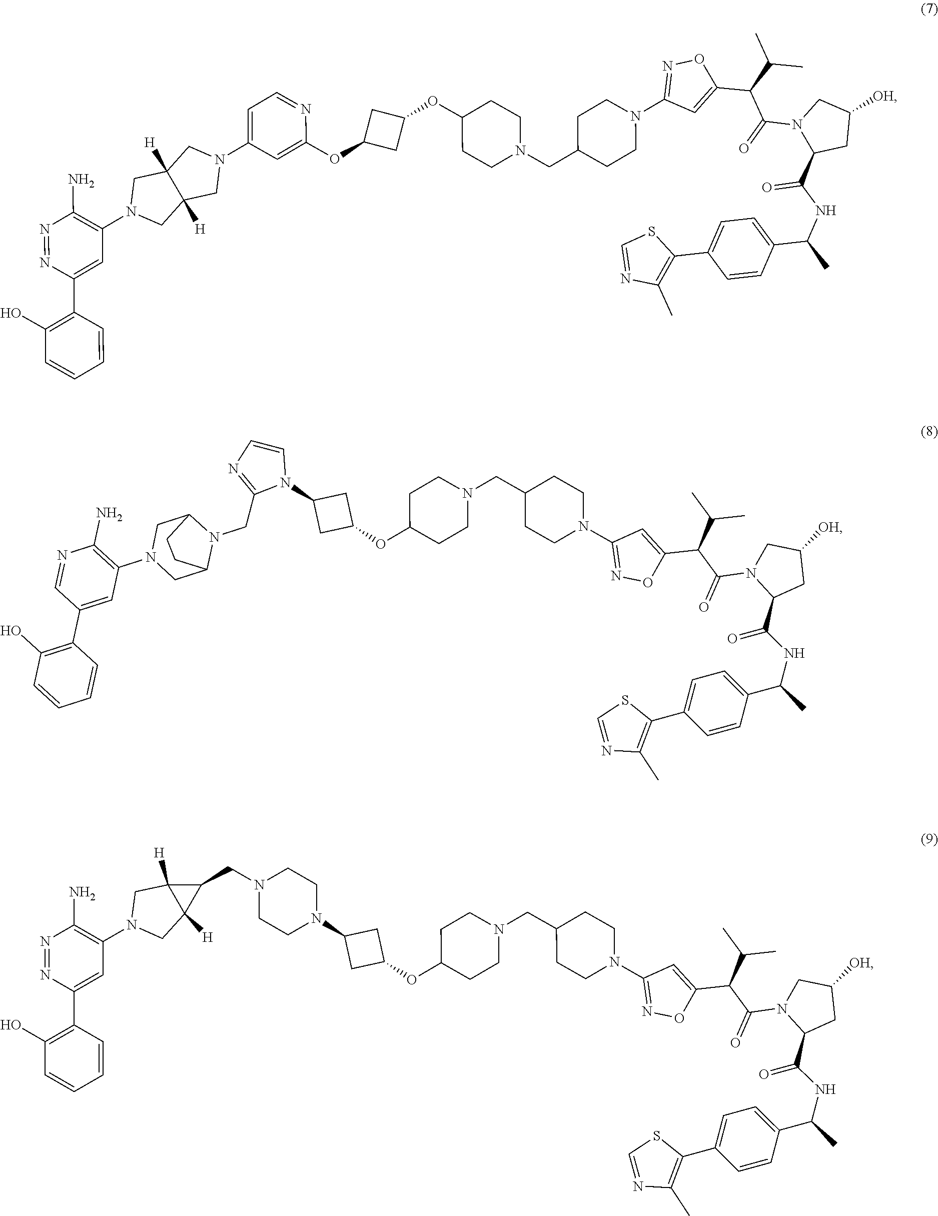 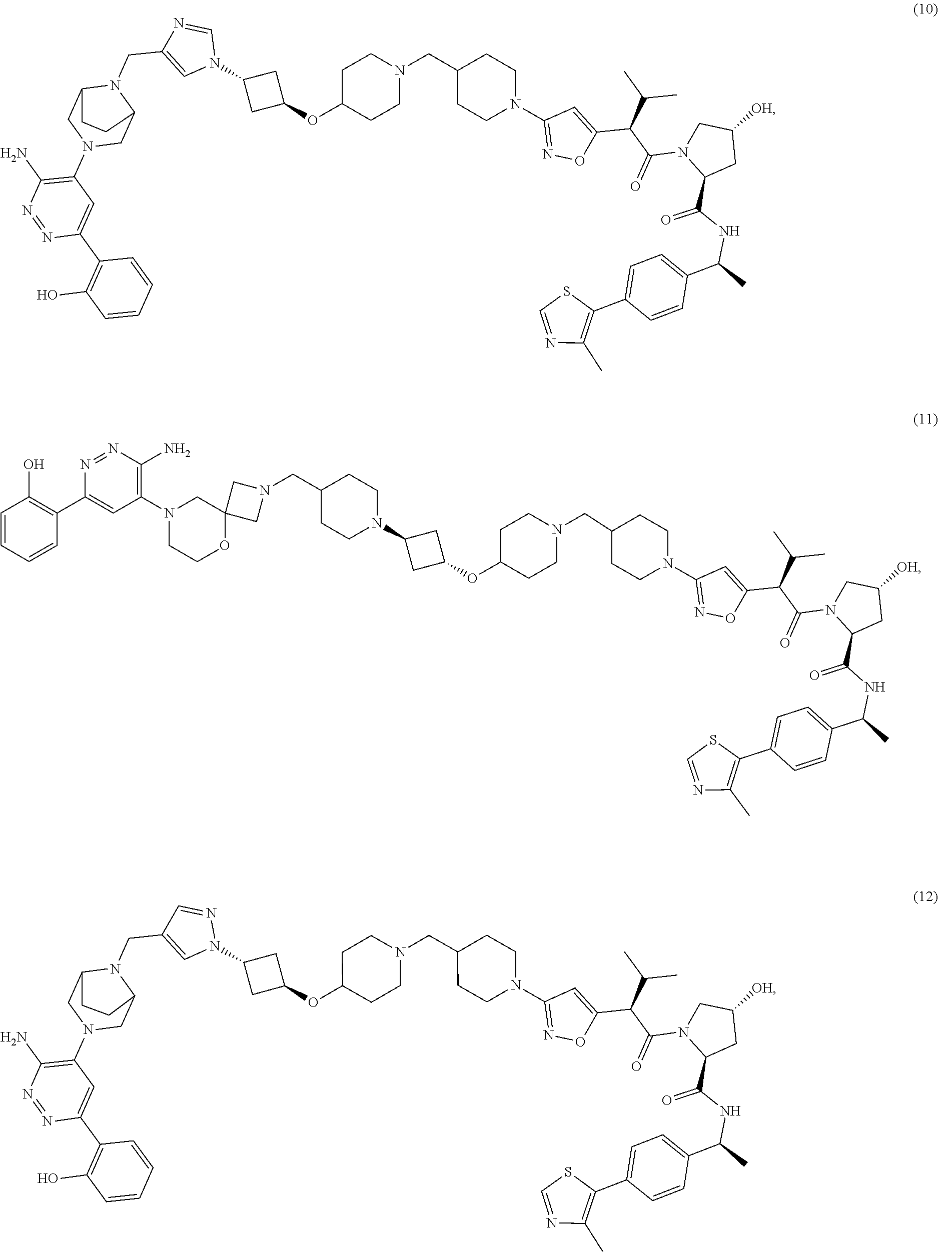 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](n6cnc(CN7C8CCC7CN(c7cc(-c9ccccc9O)nnc7N)C8)c6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(CN7CC8(C7)CN(c7cc(-c9ccccc9O)nnc7N)CCO8)CC6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](n6cc(CN7C8CCC7CN(c7cc(-c9ccccc9O)nnc7N)C8)cn6)C5)CC4)CC3)no2)C(C)C)cc1] 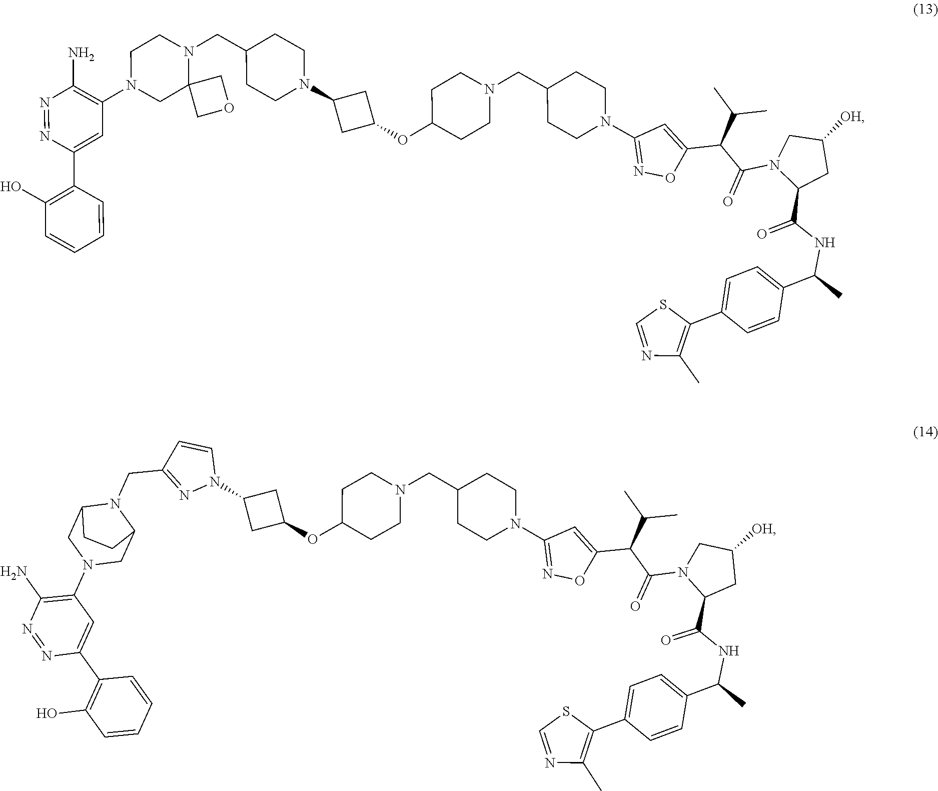 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(CN7CCN(c8cc(-c9ccccc9O)nnc8N)CC78COC8)CC6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](n6ccc(CN7C8CCC7CN(c7cc(-c9ccccc9O)nnc7N)C8)n6)C5)CC4)CC3)no2)C(C)C)cc1] 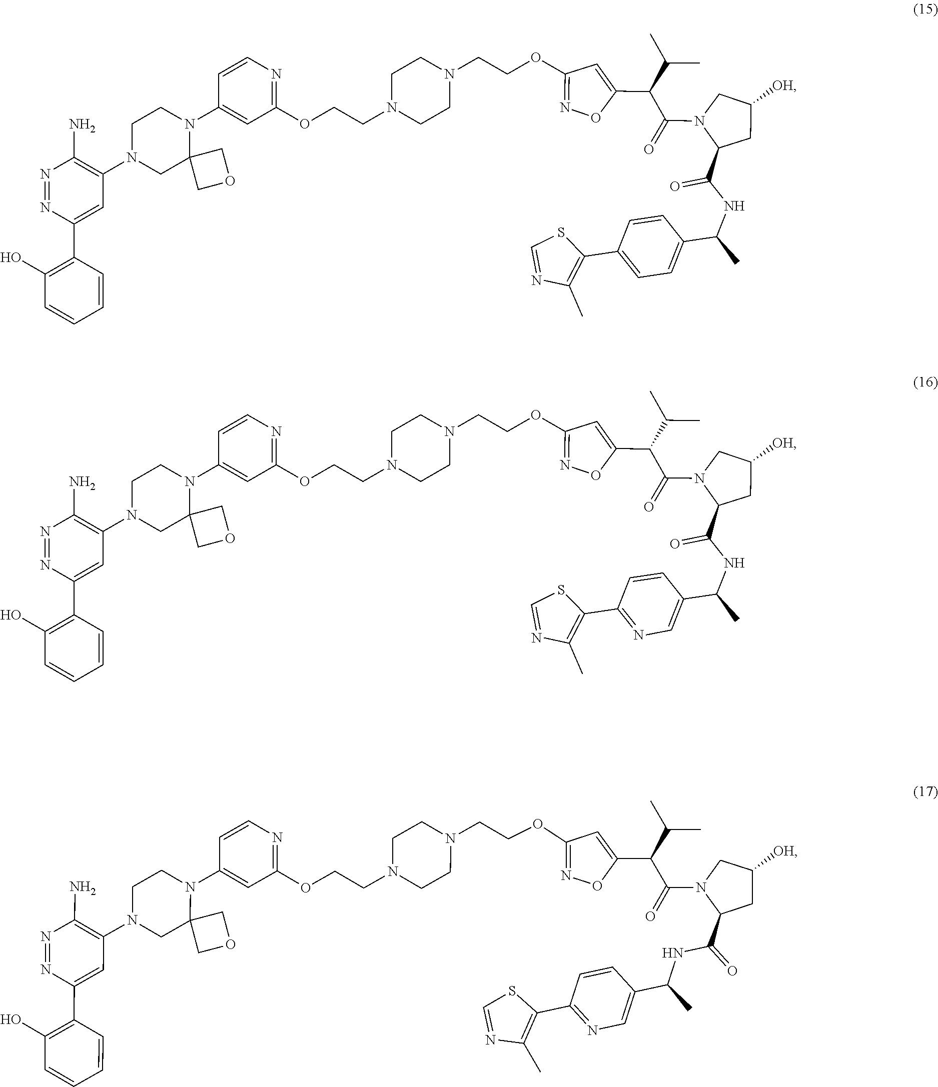 [SMILES: Cc1ncsc1-c1ccc([C@H](C)NC(=O)[C@@H]2C[C@@H](O)CN2C(=O)[C@@H](c2cc(OCCN3CCN(CCOc4cc(N5CCN(c6cc(-c7ccccc7O)nnc6N)CC56COC6)ccn4)CC3)no2)C(C)C)cc1 ; Cc1ncsc1-c1ccc([C@H](C)NC(=O)[C@@H]2C[C@@H](O)CN2C(=O)[C@H](c2cc(OCCN3CCN(CCOc4cc(N5CCN(c6cc(-c7ccccc7O)nnc6N)CC56COC6)ccn4)CC3)no2)C(C)C)cn1 ; Cc1ncsc1-c1ccc([C@H](C)NC(=O)[C@@H]2C[C@@H](O)CN2C(=O)[C@@H](c2cc(OCCN3CCN(CCOc4cc(N5CCN(c6cc(-c7ccccc7O)nnc6N)CC56COC6)ccn4)CC3)no2)C(C)C)cn1] 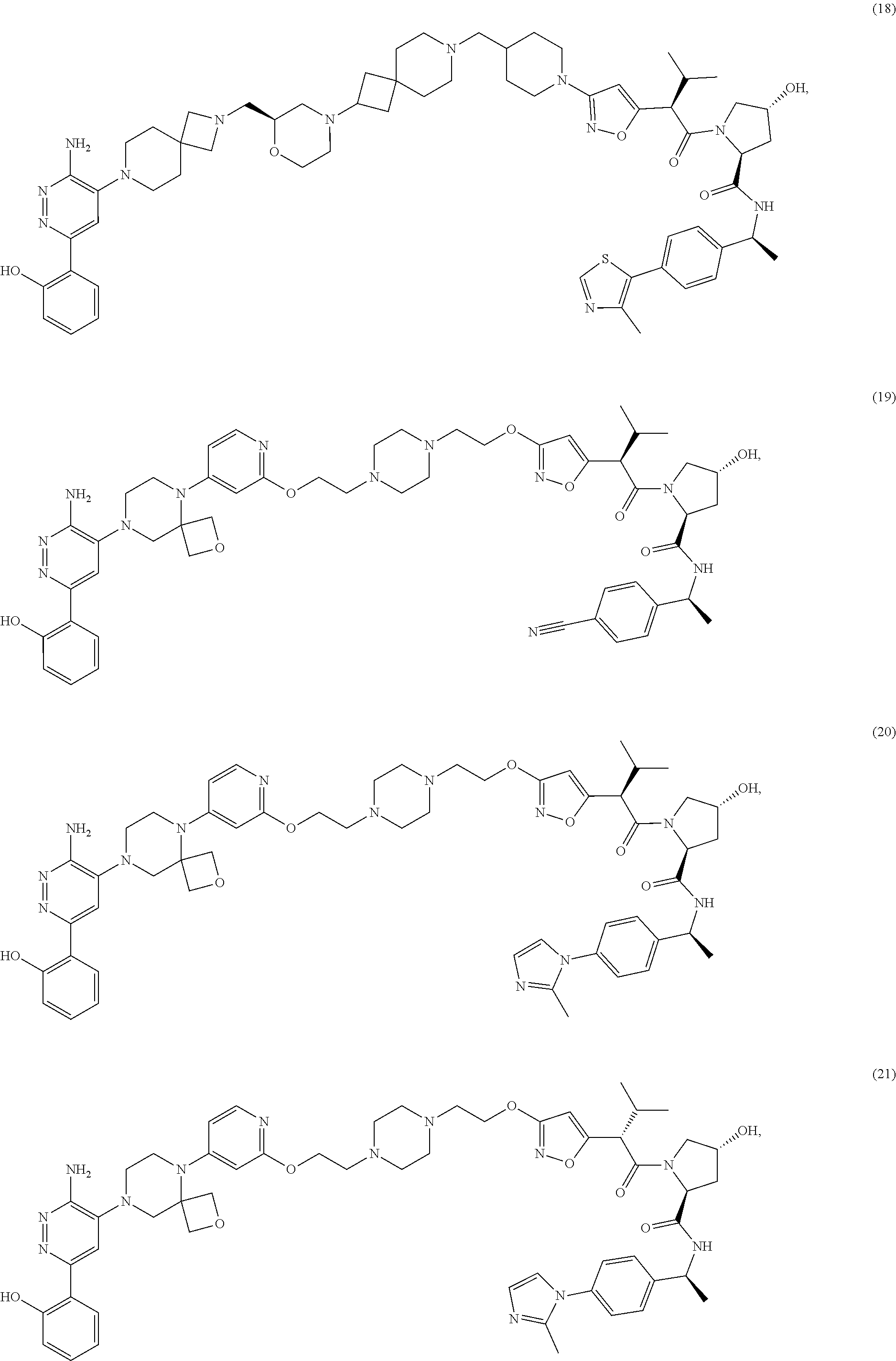 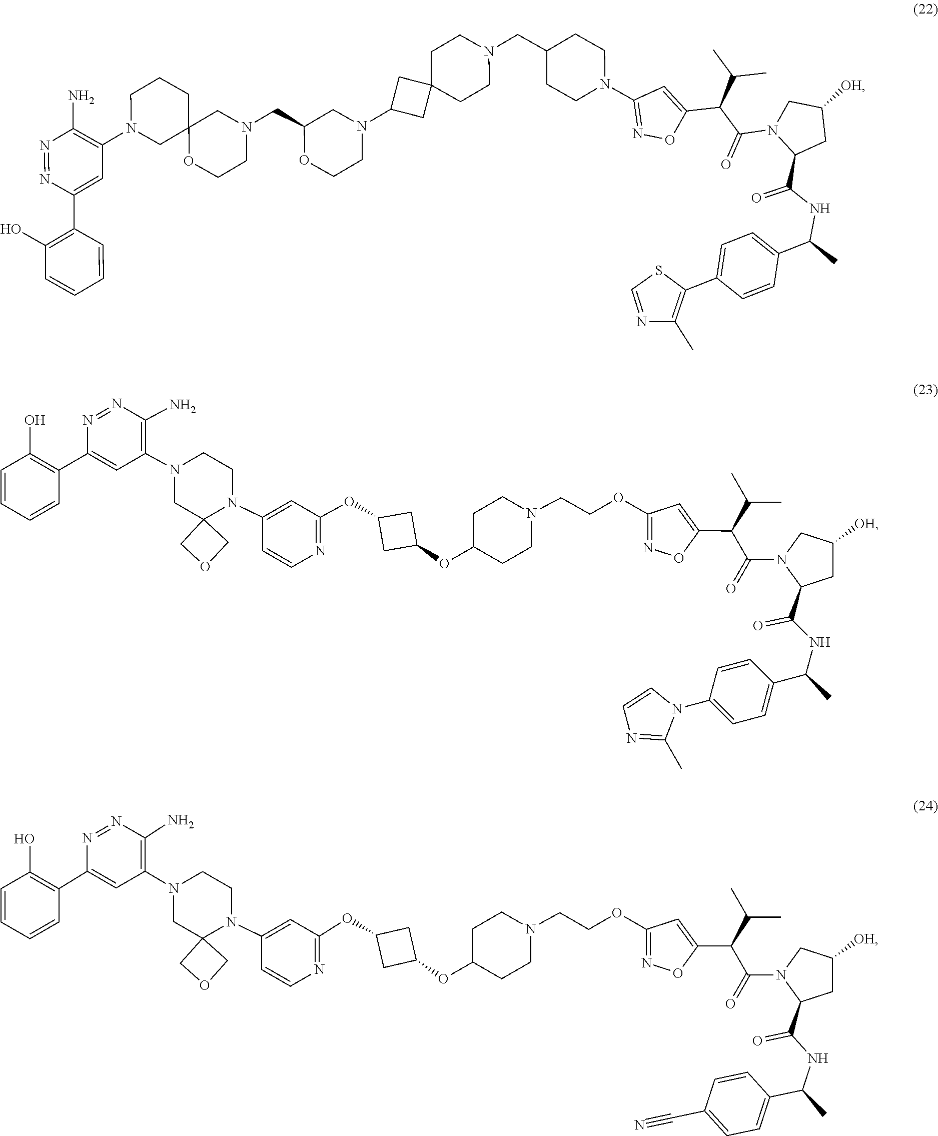 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](CN6CCOC7(CCCN(c8cc(-c9ccccc9O)nnc8N)C7)C6)C4)C5)CC3)no2)C(C)C)cc1 ; Cc1nccn1-c1ccc([C@H](C)NC(=O)[C@@H]2C[C@@H](O)CN2C(=O)[C@@H](c2cc(OCCN3CCC(O[C@H]4C[C@H](Oc5cc(N6CCN(c7cc(-c8ccccc8O)nnc7N)CC67COC7)ccn5)C4)CC3)no2)C(C)C)cc1 ; CC(C)[C@@H](C(=O)N1C[C@H](O)C[C@H]1C(=O)N[C@@H](C)c1ccc(C#N)cc1)c1cc(OCCN2CCC(O[C@H]3C[C@@H](Oc4cc(N5CCN(c6cc(-c7ccccc7O)nnc6N)CC56COC6)ccn4)C3)CC2)no1] 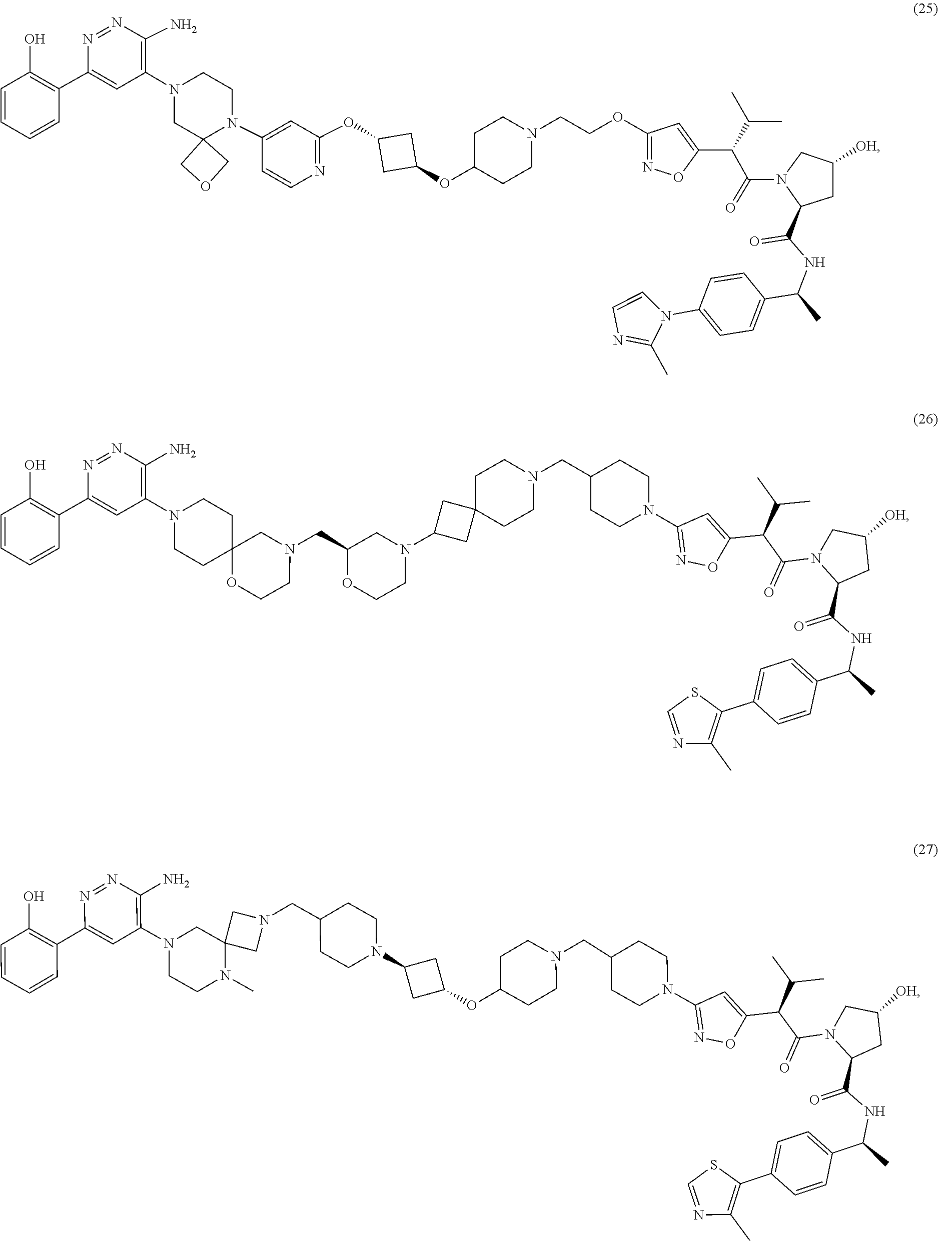 [SMILES: Cc1nccn1-c1ccc([C@H](C)NC(=O)[C@@H]2C[C@@H](O)CN2C(=O)[C@H](c2cc(OCCN3CCC(O[C@H]4C[C@H](Oc5cc(N6CCN(c7cc(-c8ccccc8O)nnc7N)CC67COC7)ccn5)C4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](CN6CCOC7(CCN(c8cc(-c9ccccc9O)nnc8N)CC7)C6)C4)C5)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(CN7CC8(C7)CN(c7cc(-c9ccccc9O)nnc7N)CCN8C)CC6)C5)CC4)CC3)no2)C(C)C)cc1] 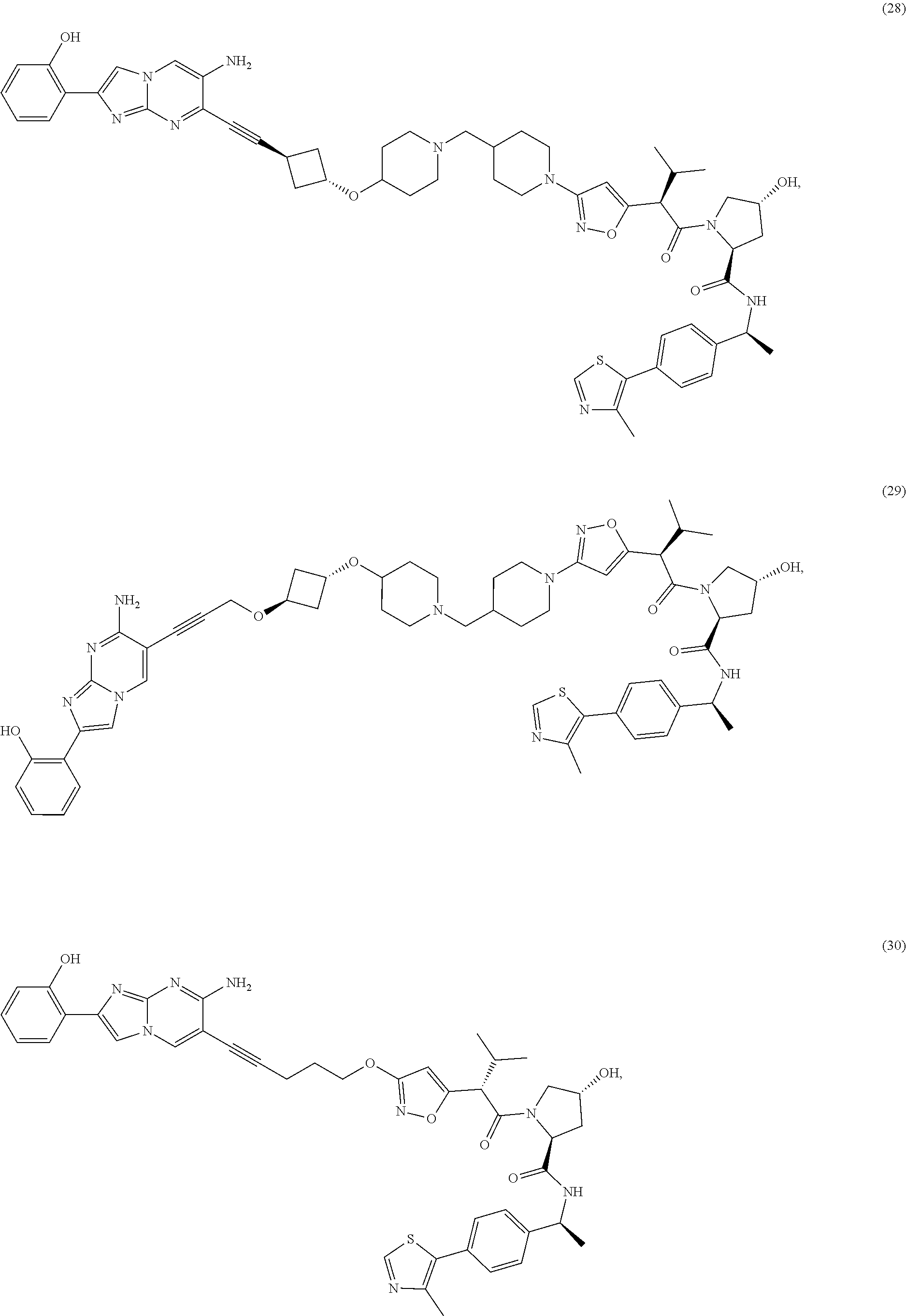 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](C#Cc6nc7nc(-c8ccccc8O)cn7cc6N)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](OCC#Cc6cn7cc(-c8ccccc8O)nc7nc6N)C5)CC4)CC3)no2)C(C)C)cc1 ; Cc1ncsc1-c1ccc([C@H](C)NC(=O)[C@@H]2C[C@@H](O)CN2C(=O)[C@H](c2cc(OCCCC#Cc3cn4cc(-c5ccccc5O)nc4nc3N)no2)C(C)C)cc1] 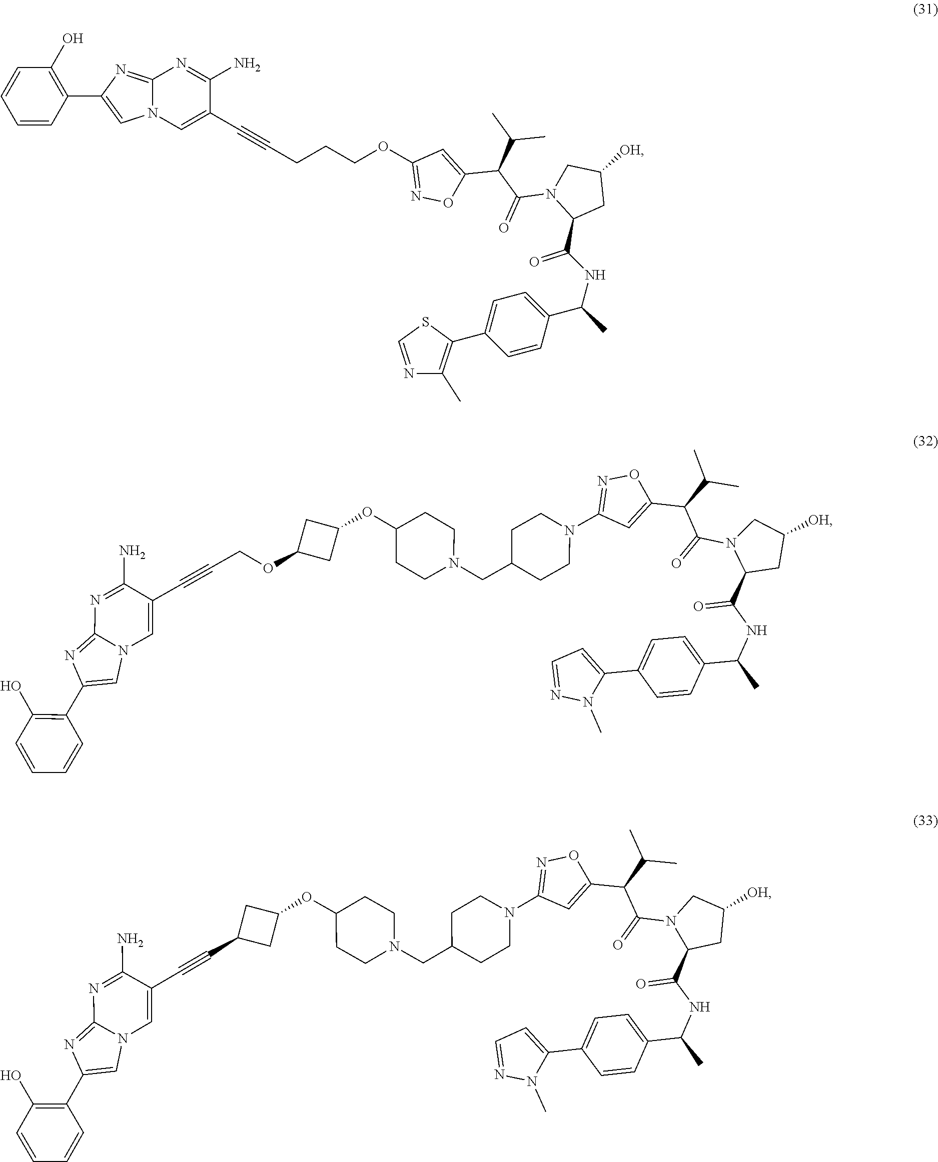 [SMILES: Cc1ncsc1-c1ccc([C@H](C)NC(=O)[C@@H]2C[C@@H](O)CN2C(=O)[C@@H](c2cc(OCCCC#Cc3cn4cc(-c5ccccc5O)nc4nc3N)no2)C(C)C)cc1 ; CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](OCC#Cc5cn6cc(-c7ccccc7O)nc6nc5N)C4)CC3)CC2)no1 ; CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](C#Cc5cn6cc(-c7ccccc7O)nc6nc5N)C4)CC3)CC2)no1] 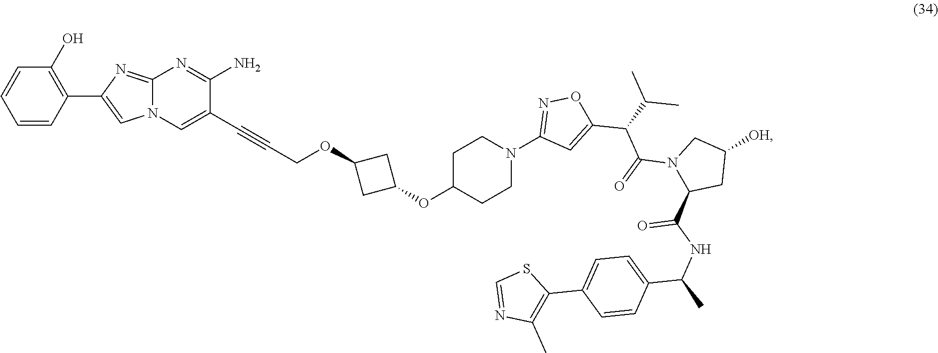 [SMILES: Cc1ncsc1-c1ccc([C@H](C)NC(=O)[C@@H]2C[C@@H](O)CN2C(=O)[C@H](c2cc(N3CCC(O[C@H]4C[C@H](OCC#Cc5cn6cc(-c7ccccc7O)nc6nc5N)C4)CC3)no2)C(C)C)cc1] 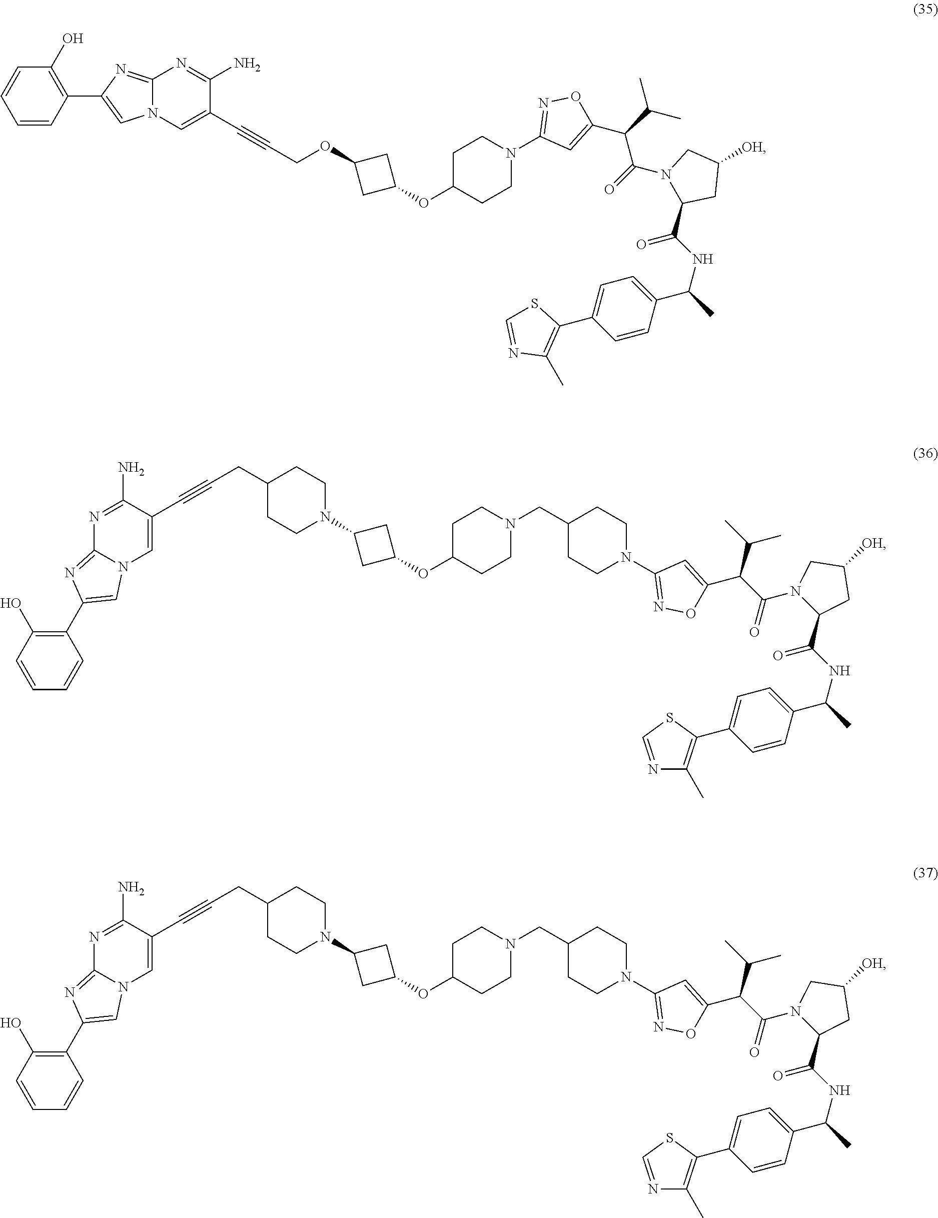 [SMILES: Cc1ncsc1-c1ccc([C@H](C)NC(=O)[C@@H]2C[C@@H](O)CN2C(=O)[C@@H](c2cc(N3CCC(O[C@H]4C[C@H](OCC#Cc5cn6cc(-c7ccccc7O)nc6nc5N)C4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@@H](N6CCC(CC#Cc7cn8cc(-c9ccccc9O)nc8nc7N)CC6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(CC#Cc7cn8cc(-c9ccccc9O)nc8nc7N)CC6)C5)CC4)CC3)no2)C(C)C)cc1] 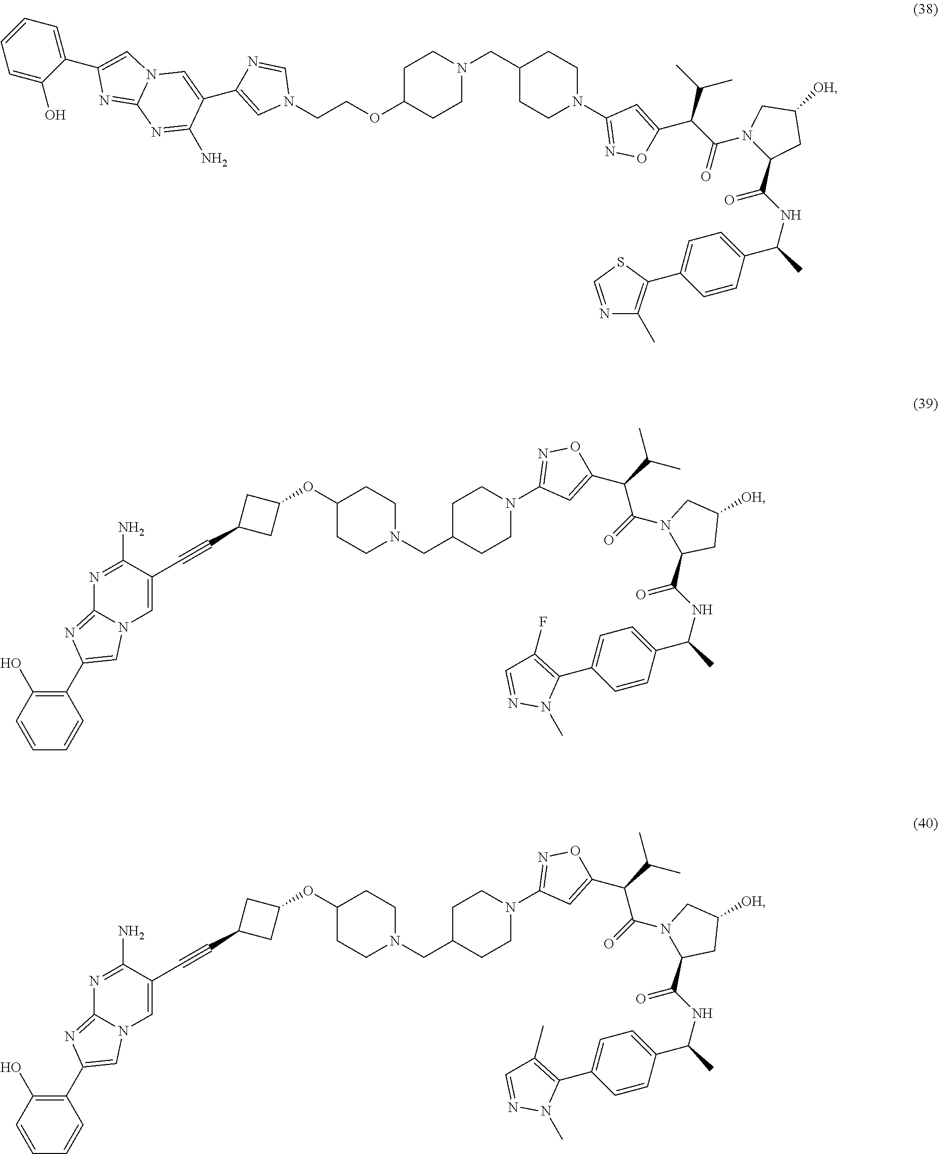 [SMILES: Cc1ncsc1-c1ccc([C@H](C)NC(=O)[C@@H]2C[C@@H](O)CN2C(=O)[C@@H](c2cc(N3CCC(CN4CCC(OCCn5cnc(-c6cn7cc(-c8ccccc8O)nc7nc6N)c5)CC4)CC3)no2)C(C)C)cc1 ; CC(C)[C@@H](C(=O)N1C[C@H](O)C[C@H]1C(=O)N[C@@H](C)c1ccc(-c2c(F)cnn2C)cc1)c1cc(N2CCC(CN3CCC(O[C@H]4C[C@H](C#Cc5cn6cc(-c7ccccc7O)nc6nc5N)C4)CC3)CC2)no1 ; Cc1cnn(C)c1-c1ccc([C@H](C)NC(=O)[C@@H]2C[C@@H](O)CN2C(=O)[C@@H](c2cc(N3CCC(CN4CCC(O[C@H]5C[C@H](C#Cc6cn7cc(-c8ccccc8O)nc7nc6N)C5)CC4)CC3)no2)C(C)C)cc1] 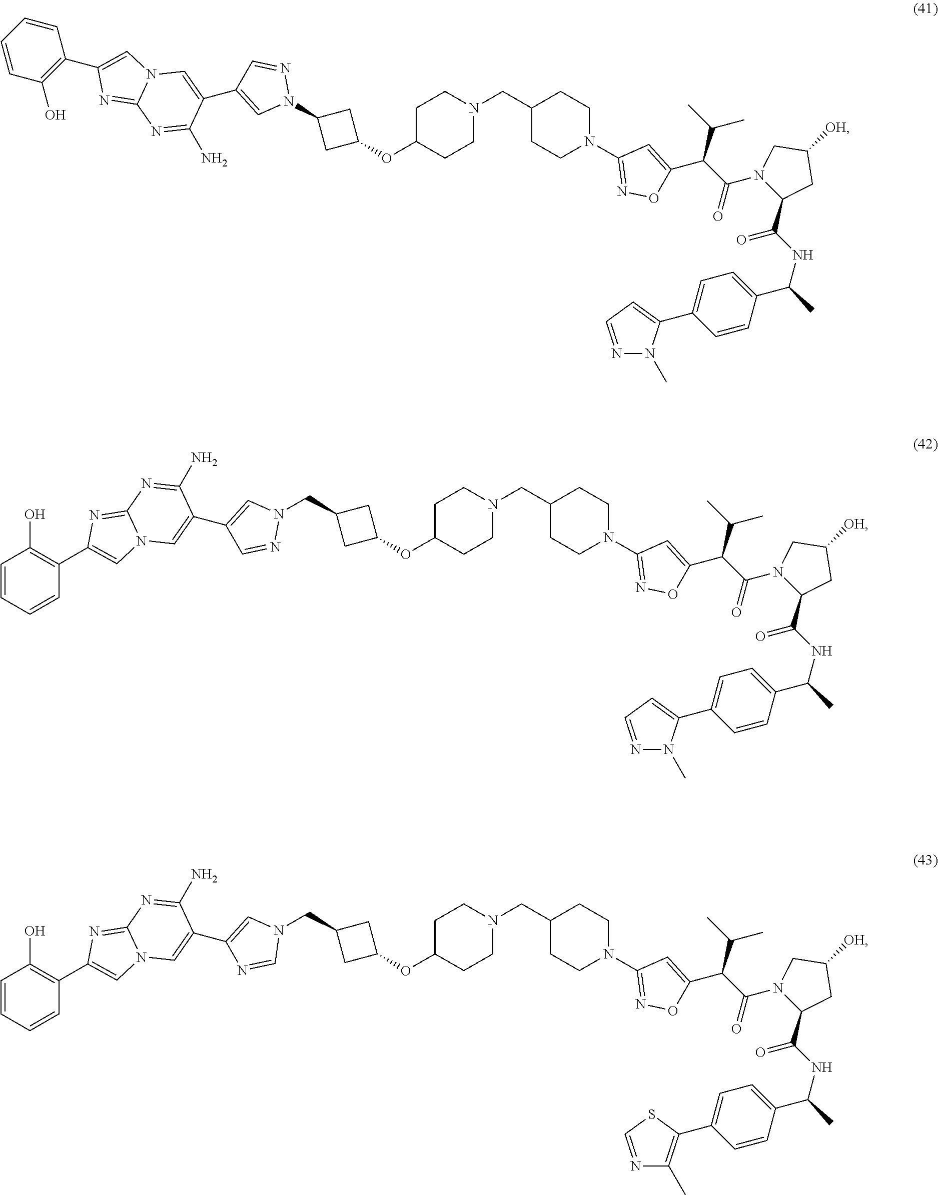 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](n5cc(-c6cn7cc(-c8ccccc8O)nc7nc6N)cn5)C4)CC3)CC2)no1 ; CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](Cn5cc(-c6cn7cc(-c8ccccc8O)nc7nc6N)cn5)C4)CC3)CC2)no1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Cn6cnc(-c7cn8cc(-c9ccccc9O)nc8nc7N)c6)C5)CC4)CC3)no2)C(C)C)cc1] 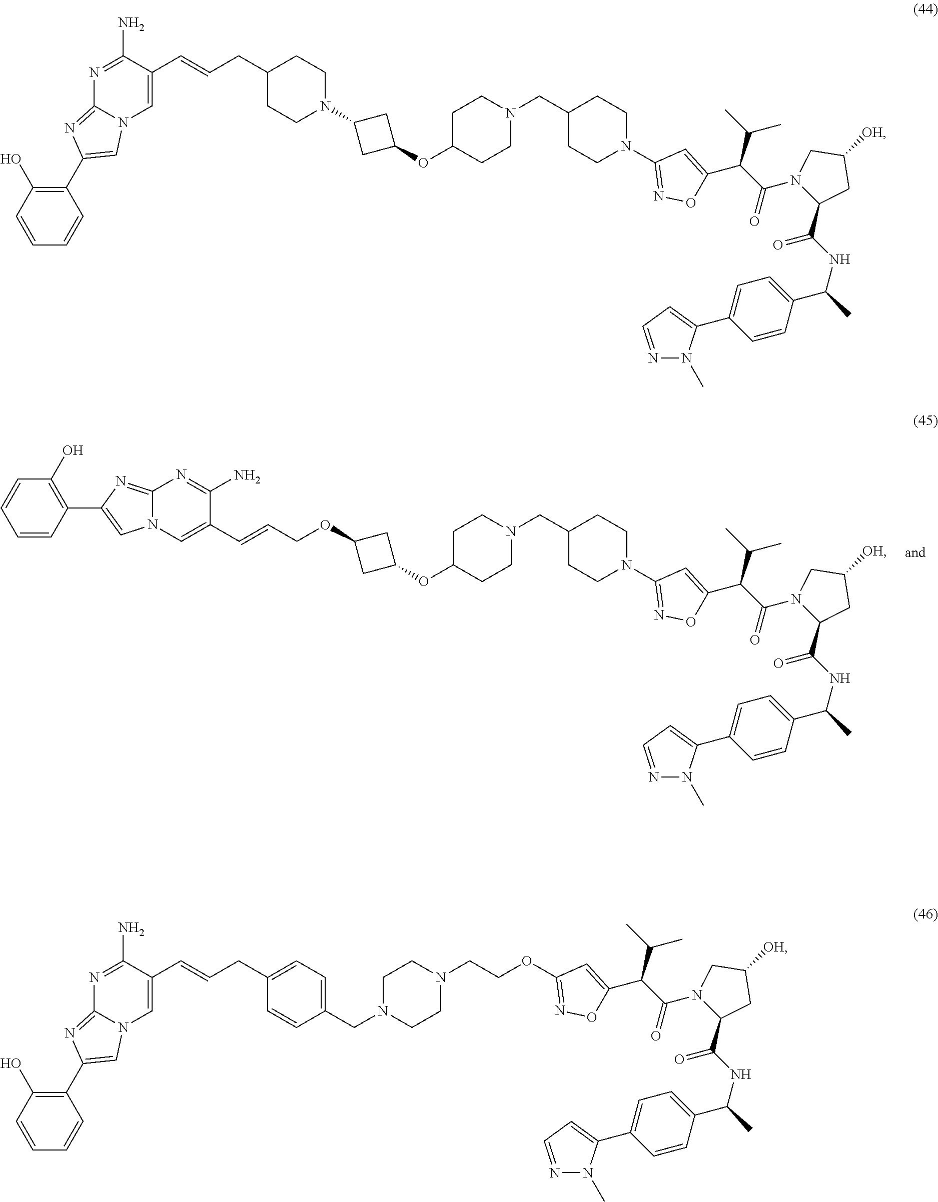 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](N5CCC(C/C=C/c6cn7cc(-c8ccccc8O)nc7nc6N)CC5)C4)CC3)CC2)no1 ; CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](OC/C=C/c5cn6cc(-c7ccccc7O)nc6nc5N)C4)CC3)CC2)no1 ; CC(C)[C@@H](C(=O)N1C[C@H](O)C[C@H]1C(=O)N[C@@H](C)c1ccc(-c2ccnn2C)cc1)c1cc(OCCN2CCN(Cc3ccc(C/C=C/c4cn5cc(-c6ccccc6O)nc5nc4N)cc3)CC2)no1]
or a pharmaceutically acceptable salt thereof.

31. The compound of claim 30, wherein at least one of: (i) the compound has a Dₘₐₓ greater than 50%, greater than 75%, or greater than or equal to 80%, (ii) the compound has a DC₅₀ less than 10 nM or less than 2.5 nM, or (iii) both (i) and (ii).

32. A compound having the chemical structure: 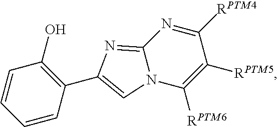
wherein:
R^{PTM4} is selected from H, —NH₂, —OH, or —NR^{PTM7}R^{PTM8}, wherein R^{PTM7} and R^{PTM8} are independently selected C₁₋₄ alkyls; and
one of R^{PTM5} and R^{PTM6} is H, and the other is selected from a H, 5- or 6-membered aryl, 5- or 6-membered heteroaryl (e.g., 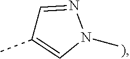
an optionally substituted 4-8-membered heterocycloalkyl (e.g. a 4-8 membered heterocycloalkyl substituted with 0, 1, or 2 substituents selected from hydroxy, halogen, alkoxy, alkyl, haloalkyl, amino, alkylamino and cyano; or 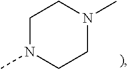
optionally substituted 8-10 membered bridged biheterocyclyl rings (e.g. a 8-10 membered bridged bicycloalkyl or bridged biheterocyclyl rings substituted with 0, 1, or 2 substituents selected from hydroxy, halogen, alkoxy, alkyl, haloalkyl, amino, alkylamino and cyano; 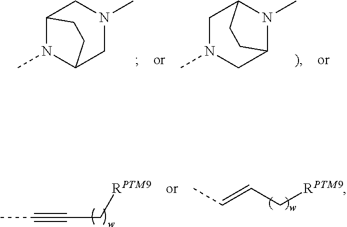
wherein W is 0, 1, or 2, and R^{PTM9} is selected from a H, alkyl (e.g., methyl), methoxy, optionally substituted 5- or 6-membered aryl, optionally substituted 5- or 6-membered heteroaryl (e.g., 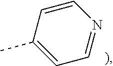
or C₁₋₆ alkylene group optionally interspaced with O atoms, provided that no heteroatom is directly attached to the double or triple carbon-carbon bond and any two heteroatoms are separated by at least two carbon atoms.

33. The compound according to claim 32, wherein the compound is selected from: 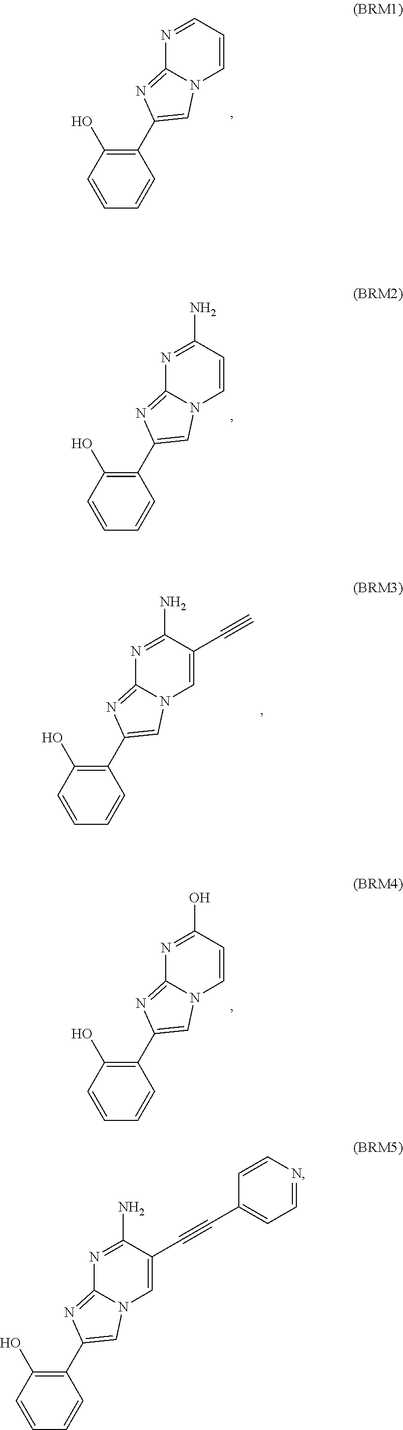 [SMILES: Oc1ccccc1-c1cn2cccnc2n1 ; Nc1ccn2cc(-c3ccccc3O)nc2n1 ; C#Cc1cn2cc(-c3ccccc3O)nc2nc1N ; Oc1ccn2cc(-c3ccccc3O)nc2n1 ; Nc1nc2nc(-c3ccccc3O)cn2cc1C#Cc1ccncc1] 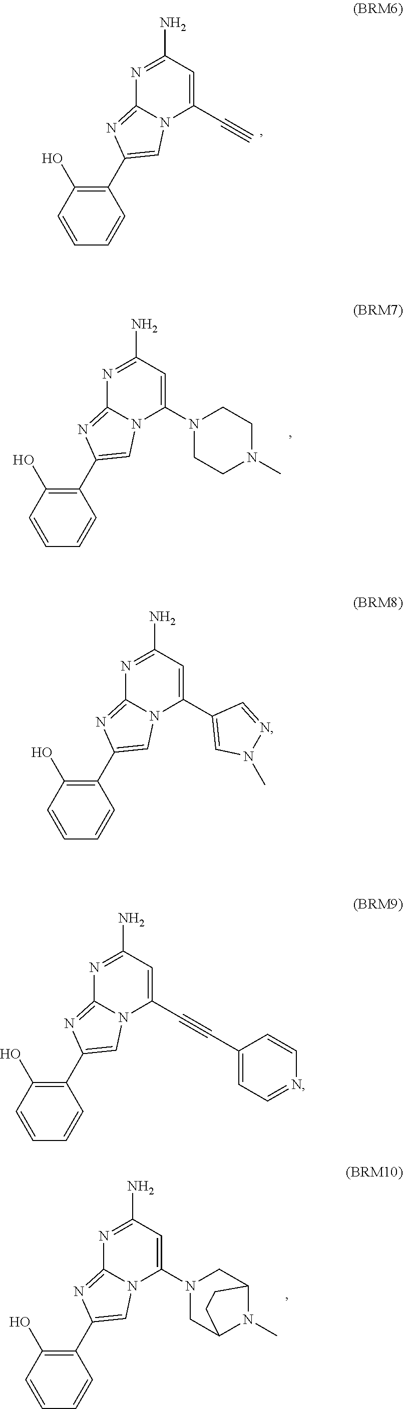 [SMILES: C#Cc1cc(N)nc2nc(-c3ccccc3O)cn12 ; CN1CCN(c2cc(N)nc3nc(-c4ccccc4O)cn23)CC1 ; Cn1cc(-c2cc(N)nc3nc(-c4ccccc4O)cn23)cn1 ; Nc1cc(C#Cc2ccncc2)n2cc(-c3ccccc3O)nc2n1 ; CN1C2CCC1CN(c1cc(N)nc3nc(-c4ccccc4O)cn13)C2] 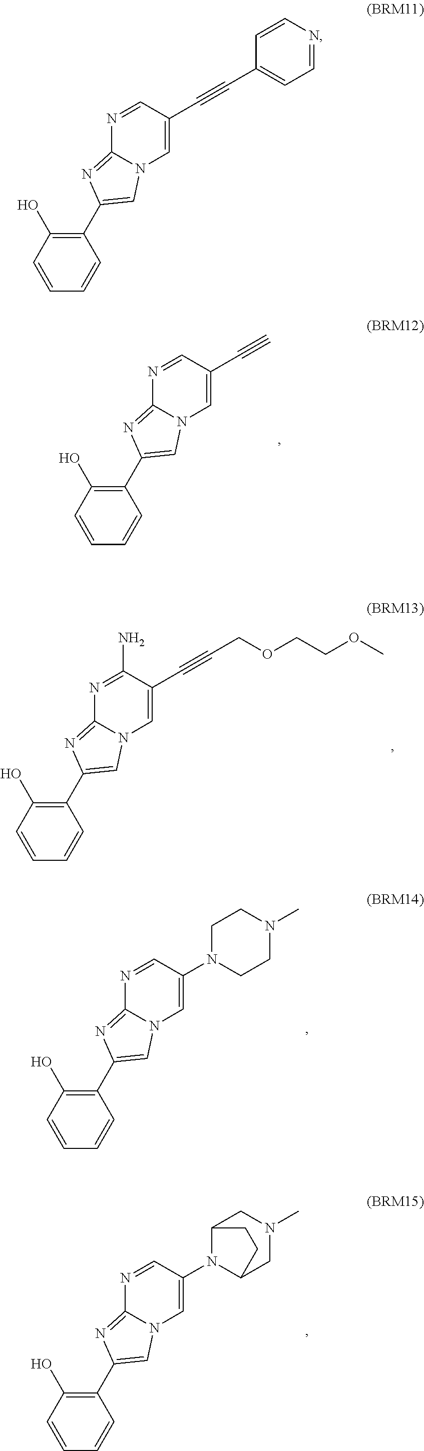 [SMILES: Oc1ccccc1-c1cn2cc(C#Cc3ccncc3)cnc2n1 ; C#Cc1cnc2nc(-c3ccccc3O)cn2c1 ; COCCOCC#Cc1cn2cc(-c3ccccc3O)nc2nc1N ; CN1CCN(c2cnc3nc(-c4ccccc4O)cn3c2)CC1 ; CN1CC2CCC(C1)N2c1cnc2nc(-c3ccccc3O)cn2c1] 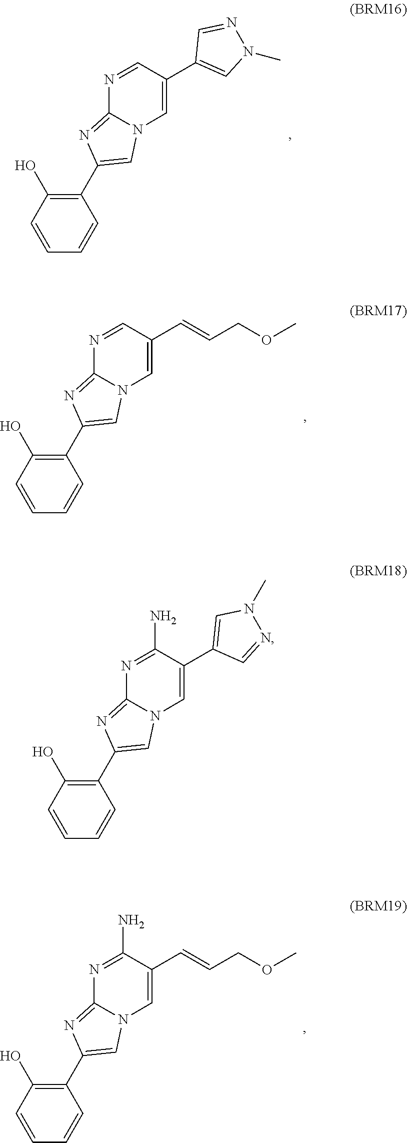 [SMILES: Cn1cc(-c2cnc3nc(-c4ccccc4O)cn3c2)cn1 ; COC/C=C/c1cnc2nc(-c3ccccc3O)cn2c1 ; Cn1cc(-c2cn3cc(-c4ccccc4O)nc3nc2N)cn1 ; COC/C=C/c1cn2cc(-c3ccccc3O)nc2nc1N]
and pharmaceutically acceptable salts thereof.

34. A pharmaceutical composition comprising an effective amount of a compound of any of claims 1-33, a pharmaceutically acceptable carrier.

35. The pharmaceutical composition of claim 34, further comprising an additional anti-cancer agent.

36. A composition comprising a pharmaceutically acceptable carrier and an effective amount of at least one compound of any of claims 1-33 for treating a disease or disorder in a subject, the method comprising administering the composition to a subject in need thereof, wherein the compound is effective in treating or ameliorating at least one symptom of the disease or disorder, wherein the disease or disorder is associated with SMARCA1, BRAHMA or BRM accumulation and aggregation.

37. The composition of claim 36, wherein the disease or disorder is cancer.

38. The composition of claim 37, wherein the cancer is a SWI/SNF associated cancer or a cancer with a SMARCA4 mutation.

39. The composition of claim 38, wherein the SWI/SNF associated cancer or the cancer with a SMARCA4 mutation is lung cancer or non-small cell lung cancer.

40. The composition of claim 36, wherein the cancer is an SMARCA4-deficient cancer or a cancer with decreased expression of SMARCA4 relative to normal SMARCA4 expression.

41. The composition of claim 40, wherein the SMARCA4-deficient cancer or the cancer with decreased expression of SMARCA4 relative to normal SMARCA4 expression is lung cancer or non-small cell lung cancer.
